(19) United States
(12) Patent Application Publication
(10) Pub. No.: US 2026/0250634 A1
(43) Pub. Date: Aug. 27, 2026

(54) GENE-EDITED NATURAL KILLER CELLS

(71) Applicant: CRISPR THERAPEUTICS AG, Zug (CH)

(72) Inventors: Valentin SLUCH, South Boston, MA (US); Alireza REZANIA, South Boston, MA (US); Tiansu WANG, South Boston, MA (US); Viktoriia KYRYCHENKO, South Boston, MA (US); Danielle SWAIN, South Boston, MA (US); Meichen LIAO, South Boston, MA (US)

(21) Appl. No.: 18/870,641

(22) PCT Filed: Jun. 1, 2023
(86) PCT No.: PCT/IB2023/055621

§ 371 Date: Nov. 30, 2024

Related U.S. Application Data
(60) Provisional application No. 63/347,959, filed on Jun. 1, 2022.
(60) Provisional application No. 63/347,971, filed on Jun. 1, 2022.

(51) Int. Cl. C12N 5/0783; A61K 9/00; A61K 35/17; A61K 40/11; A61K 40/13; A61K 40/15; A61K 40/42; A61P 35/00; C07K 14/54; C07K 14/705; C07K 14/715; C12N 5/0735; C12N 5/074; C12N 5/0781; C12N 5/0789; C12N 9/22; C12N 15/113; C12N 15/62; C12N 15/63

(52) U.S. Cl. CPC C12N 5/0646; A61K 9/0019; A61K 35/17; A61K 40/11; A61K 40/13; A61K 40/15; A61K 40/42; A61P 35/00; C07K 14/5443; C07K 14/70535; C07K 14/70539; C07K 14/7155; C12N 5/0606; C12N 5/0635; C12N 5/0636; C12N 5/0647; C12N 5/0696; C12N 9/22; C12N 15/1138; C12N 15/62; C12N 15/63; A61K 2239/31; A61K 2239/53; A61K 2239/55; C07K 2319/00; C12N 2506/45; C12N 2510/00

(57) ABSTRACT

The present disclosure relates to, inter alia, an engineered cell (e.g., iPSC, IPS-derived immune cells, e.g., NK or T cells, or NK cells or T cells”) comprising a disrupted B2M gene, a disrupted CIITA gene, an inserted polynucleotide encoding HLA-E, an inserted polynucleotide encoding a fusion of IL15 and IL15Rα, an inserted polynucleotide encoding SERPINB9, and a polynucleotide encoding a CAR, e.g., an anti-GPC3 CAR or an anti-GPR87 CAR. The engineered cell may further comprise a disrupted FAS gene, a disrupted CISH gene, a disrupted CD38 gene, a disrupted FLI1 gene, a disrupted TGFBR1 gene, a disrupted TGFBR2 gene, an inserted polynucleotide encoding CD16, an inserted polynucleotide encoding CD64, and/or an inserted polynucleotide encoding an NKG2D CAR. Methods for producing the engineered cells are also provided, and therapeutic uses of the engineered cells are also described.

### RELATED APPLICATIONS

The present application is a U.S. national phase application under 35 U.S.C. § 371 of International Application No. PCT/IB2023/055621, filed on Jun. 1, 2023 and published as WO 2023/233342 A2 on Dec. 7, 2023, which claims the benefit under 35 U.S.C. § 119 (e) to U.S. Provisional Patent Application No. 63/347,959, filed Jun. 1, 2022; and U.S. Provisional Application No. 63/347,971, filed Jun. 1, 2022. The contents of these related applications are incorporated herein by reference in their entireties for all purposes.

### REFERENCE TO SEQUENCE LISTING

The present application is being filed along with a Sequence Listing in electronic format. The Sequence Listing is provided as a file entitled 80EM-341749-US_SequenceListing, created May 16, 2023, which is 350 kilobytes in size. The information in the electronic format of the Sequence Listing is incorporated herein by reference in its entirety.

### BACKGROUND

### Field

The disclosure relates to the field of gene-edited iPSC and Natural Killer (NK) cells.

### Description of the Related Art

There is a need for adoptive cell therapy that does not rely on the use of cells obtained from patients or donors and does not induce allogeneic rejection. Natural Killer (NK) cells are potent anti-tumor effectors, making them attractive candidates for cancer immunotherapy. However, the use of NK cells, in particular NK cells expressing a chimeric antigen receptor (CAR), for adoptive cell therapy remains to be challenging. For example, there is a need to improve the efficacy, persistence, cytotoxic activity, immune evasion and tumor targeting of therapeutic NK cells. There is also a need for a uniform pool of therapeutic NK cells that can be manufactured in a consistent manner for use in any patients in need thereof.

### SUMMARY

In some aspects, the present disclosure provides engineered cells that have been edited using, for example, CRISPR/Cas9 gene editing technology.

In some aspects, the present disclosure provides engineered cells comprising a disrupted beta-2-microglobulin (B2M) gene, a disrupted Class II Transactivator (CIITA) gene, a disrupted Cytokine Inducible SH2 Containing Protein (CISH) gene, a disrupted Fas Cell Surface Death Receptor (FAS) gene, a disrupted CD38 (also called cyclic ADP ribose hydrolase), a disrupted Friend Leukemia Integration 1 (FLI1) gene, a disrupted Transforming Growth Factor Beta Receptor 2 (TGFBR2) gene, a disrupted Transforming Growth Factor Beta Receptor 1 (TGFBR1) gene, disrupted Zinc-finger E homeobox-binding-1 (ZEB1) gene, a disrupted receptor of programmed cell death protein 1 (PD-1) gene, a disrupted T cell immunoreceptor with Ig and ITIM domains (TIGIT) gene, a disrupted REGNASE-1 gene, a disrupted Natural Killer Group Protein 2A (NKG2A) gene, a disrupted Adenosine receptor 2a or 2b (ADOR2A/ADOR2B) gene, a disrupted ADAM metallopeptidase domain 17 (ADAM17) gene and/or an insertion of a polynucleotide encoding an IL15/IL15Rα fusion, an insertion of a polynucleotide encoding SERPINB9, an insertion of a poly nucleotide encoding Major Histocompatibility Complex, Class I, E (HLA-E), an insertion of a polynucleotide encoding an anti-glypican 3 (GPC3) CAR, an insertion of a polynucleotide encoding an anti-G protein-coupled receptor 87 (GPR87) CAR, an insertion of a polynucleotide encoding CD16 (also known as FcγRIII), and/or an insertion of a polynucleotide encoding CD64 (also known as FcγRI), an insertion of a polynucleotide encoding an Natural Killer Group Protein 2D (NKG2D) CAR, an insertion of a polynucleotide encoding an anti-CD19-CD20-BCMA CAR, an insertion of a polynucleotide encoding an anti-glycoprotein A33 (A33) CAR, an insertion of polynucleotide encoding an anti-CD33 (also known as p67 or SIGLEC3) CAR, an insertion of a polynucleotide encoding an anti-CD30 CAR, wherein the cell expresses the IL15/IL15Rα fusion, SERPINB9, HLA-E, anti-GPC3 CAR, anti-GPR87 CAR, CD16, CD64, anti-CD19-CD20-BCMA CAR, anti-A33 CAR, anti-CD33 CAR, NKG2D CAR and/or anti-CD30 CAR, and the cell has disrupted expression of B2M, CIITA, FAS, CISH, CD38, FLI1, TGFBR2. ZEB1, PD-1, TIGIT, REGNASE-1, NKG2A, ADAM17, and/or A2a/A2b. In some embodiments, disrupted expression comprises reduced or eliminated expression of said gene product.

Also provided include engineered cells comprising (a) a disrupted B2M gene, and (b) an insertion of a first polynucleotide encoding SERPINB9 and a second polynucleotide encoding an IL15/IL15Rα fusion, optionally wherein the first polynucleotide and the second polynucleotide are inserted in the disrupted B2M gene, (c) a disrupted CIITA gene; (d) an insertion of a third polynucleotide encoding a CAR and a fourth polynucleotide encoding HLA-E, wherein the CAR is an anti-GPC3 CAR or an anti-GPR87 CAR, optionally wherein the CAR and HLA-E are inserted in the disrupted CIITA gene; (e) a disrupted CISH gene, and (f) a disrupted FAS gene; wherein the cell expresses SERPINB9, the IL15/IL15Rα fusion, HLA-E, and the anti-GPC3 CAR or the anti-GPR87 CAR, and the cell has a disrupted expression of B2M, CIITA, CISH, and FAS.

The engineered cell can further comprise (g) a disrupted CD38 gene, (h) a disrupted FLI1 gene, a disrupted TGFBR2 gene, a disrupted TGFBR1 gene, an insertion of a polynucleotide encoding CD16, an insertion of a polynucleotide encoding CD64, and/or an insertion of a polynucleotide encoding an NKG2D CAR, wherein the cell expresses CD16, CD64, and/or the NKG2D CAR and has disrupted expression of CD38, FLI1, TGFBR1, and/or TGFBR2.

In some embodiments, the first polynucleotide and second polynucleotide are inserted as SERPINB9-P2A-IL15/IL15Rα construct, wherein the SERPINB9-P2A-IL15/IL15Rα construct encodes SERPINB9, a P2A peptide, and the IL15/IL15Rα fusion. In some embodiments, the polynucleotide encoding SERPINB9-P2A-IL15/IL15Rα is inserted in exon 1 of the B2M gene locus, thereby disrupting the B2M gene. In some embodiments, the third polynucleotide and the fourth polynucleotide are inserted as a CAR-P2A-HLA-E construct, wherein CAR-P2A-HLA-E construct encodes the CAR (either anti-GPC3 CAR or anti-GPR87 CAR), a P2A peptide, and HLA-E. In some embodiments, HLA-E is an HLA-E trimer comprising a B2M signal peptide fused to an HLA-G presentation peptide fused to the B2M membrane protein fused to the HLA-E protein without a signal peptide. In some embodiments, the polynucleotide encoding CAR-P2A-HLA-E is inserted in exon 2 of the CIITA gene locus, thereby disrupting the CIITA gene. In some embodiments, the polynucleotide encoding NKG2D CAR is inserted into a second location in the B2M gene locus. In some embodiments, the polynucleotide encoding CD16 is inserted into any of the CISH, FAS, FLI1, TGFBR1, or TGFBR2 gene loci, thereby disrupting said gene locus. In some embodiments, the polynucleotide encoding CD64 is inserted into any of the CISH, FAS, FLI1, TGFBR1 or TGFBR2 gene loci, thereby disrupting said gene locus.

In some embodiments, the engineered cell comprises a disrupted FAS, CISH, CD38, FLI1, TGFBR1 and/or TGFBR2 gene. In some embodiments, the disrupted expression of FAS comprises reduced expression of FAS. In some embodiments, the disrupted expression of FAS comprises eliminated expression of FAS. In some embodiments, the disrupted expression of CISH comprises reduced expression of CISH. In some embodiments, the disrupted expression of CISH comprises eliminated expression of CISH. In some embodiments, the disrupted expression of CD38 comprises reduced expression of CD38. In some embodiments, the disrupted expression of CD38 comprises eliminated expression of CD38. In some embodiments, the disrupted expression of FLI1 comprises reduced expression of FLI1. In some embodiments, the disrupted expression of FLI1 comprises eliminated expression of FLI1. In some embodiments, the disrupted expression of TGFBR1 comprises reduced expression of TGFβR1. In some embodiments, the disrupted expression of TGFβR1 comprises eliminated expression of TGFβR1. In some embodiments, the disrupted expression of TGFβR2 comprises reduced expression of TGFβR2. In some embodiments, the disrupted expression of TGFβR2 comprises eliminated expression of TGFBR2. In some embodiments, the engineered cell comprises disrupted expression of FAS, CISH, CD38, FLI1, TGFBR1, and/or TGFBR2.

The engineered cell can be a stem cell. In some embodiments, the stem cell is an induced pluripotent stem cell (iPSC), a hematopoietic stem cell, an embryonic stem cell, or an adult stem cell. In some embodiments, the engineered cell is a genome-edited iPSC. In some embodiments, the engineered cell is a natural killer (NK) cell obtained from a genome-edited iPSC (also known as “IPS-derived NK” and “INK” cells). In some embodiments, the engineered cell is a differentiated cell or a somatic cell. In some embodiments, the engineered cell is capable of being differentiated into lineage-restricted progenitor cells or fully differentiated somatic cells. In some embodiments, engineered cell is a natural killer (NK) cell. In some embodiments, the NK cell has been differentiated from a genome-edited iPSC, wherein the NK cell comprises the genome edits of the genome-edited iPSC, wherein the NK cell has not been genome-edited after the differentiation.

In some embodiments, the engineered cell expresses at least one, two, three, four or five of the following markers: CD56, NKp44, NKp46, CD94, NKG2A and KIR2DL4, and optionally wherein the markers are expressed at least at 25%, 30%, 40%, 50%, or 75% level relative to their expression in wild type NK cells.

In some embodiments, the engineered cell has at least one of the following characteristics, or any combination thereof: (i) persistency, (ii) immune evasiveness, (iii) lack of an alloimmune T cell response, (iv) cytotoxic activity, (v) ADCC activity, and (vi) anti-tumor activity. In some embodiments, the engineered cell has at least one of the following characteristics, or any combination thereof: (i) improved persistency, (ii) improved immune evasiveness, (iii) improved cytotoxic activity, (iv) improved ADCC activity, and (v) improved anti-tumor activity; wherein the characteristics are improved relative to a wild-type cell, optionally, relative to a wild-type iPSC or a wild-type NK cell. In some embodiments, the engineered cell has at least one of the following characteristics, or any combination thereof: (i) improved persistency, (ii) improved immune evasiveness, (iii) improved cytotoxic activity, (iv) improved ADCC activity, and (v) improved anti-tumor activity; wherein the characteristics are improved relative to a wild-type cell, optionally, relative to an unmodified iPSC or an unmodified NK cell. In some embodiments, the engineered cell has at least one of the following characteristics, or any combination thereof: (i) an alloimmune T cell reaction of less than 10% relative to an unmodified cell, and (ii) cytotoxic activity resulting in killing more than 50% of target cells when the engineered cells are mixed with the target cells at the ratio of 1:1; (iii) at least 50% increase in cellular viability relative to an unmodified cell. In some embodiments, the engineered cell has at least one of the following characteristics, or any combination thereof: (i) an alloimmune T cell reaction of less than 50%, 40%, 30%, 20%, 10% or 5% relative to an unmodified cell, and (ii) cytotoxic activity resulting in killing more than 30%, 40%, 50%, 60%, 70% or 80% of target cells when the engineered cells are mixed with the target cells at the ratio of 1:1; (iii) at least 30%, 40%, 50%, 60%, 70% or 80% increase in cellular viability relative to an unmodified cell. In some embodiments, the engineered cell is immune evasive as measured by an alloimmune T cell reaction of less than 15%, less than 10%, less than 9%, less than 8%, less than 7.5%, less than 7%, less than 6%, less than 5%, less than 4%, or less than 3% (relative to an unmodified or WT cell). In some embodiments, the engineered cell is immune evasive as measured by an alloimmune T cell reaction of less than 10% (relative to an unmodified or WT cell). In some embodiments, the engineered cell is immune evasive as measured by an alloimmune T cell reaction of less than 5% (relative to an unmodified or WT cell). In some embodiments, the engineered cell is cytotoxic as measured by the killing of more than 30%, more than 40%, more than 50%, more than 60%, more than 70%, more than 75% or more than 80% of target cells, when the engineered cells are mixed with the target cells at the ratio of 1:1. In some embodiments, the engineered cell is cytotoxic as measured by the killing of more than 75% of target cells, when the engineered cells are mixed with the target cells at the ratio of 1:1. In some embodiments, the engineered cell is cytotoxic as measured by the killing of more than 75%, more than 80%, more than 85%, more than 90%, more than 95%, more than 98% or more than 99% of target cells, when the engineered cells are mixed with the target cells at the ratio of 4:1. In some embodiments, the engineered cell is cytotoxic as measured by the killing of more than 80% of target cells, when the engineered cells are mixed with the target cells at the ratio of 4:1. In some embodiments, the engineered cell is cytotoxic as measured by the killing of more than 95% of target cells, when the engineered cells are mixed with the target cells at the ratio of 4:1. In some embodiments, the engineered cell is capable of cell expansion in the absence of exogenous IL 15 in cell culture media.

Also provided herein include a plurality of any of the engineered cells described herein. In further aspects, the present disclosure provides a population of lineage-restricted progenitor cells or fully differentiated somatic cells derived from the plurality of engineered cells described herein. In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells are pancreatic beta cells, epithelial cells, endodermal cells, macrophages, hepatocytes, adipocytes, kidney cells, blood cells, cardiomyocytes, or immune system cells. In some embodiments, the hematopoietic cells are NK cells, T cells, B cells, or NKT cells. The fully differentiated cells can be human NK cells.

In some embodiments, at least 25% or at least 50% of engineered cells of the population express SERPINB9, the IL15/IL15Rα fusion, the anti-GPC3 or anti-GPR87 CAR, HLA-E, CD16, CD64, and/or the NKG2D CAR. In some embodiments, at least 50% of engineered cells of the population do not express a detectable level of B2M protein, CIITA protein, FAS protein, CISH protein, CD38 protein, and/or TGFBR2 protein.

In some embodiments, the engineered human NK cells of the population, when co-cultured in vitro with a population of cancer cells, induce cell lysis of at least 70%, at least 80%, or at least 90% of the population of cancer cells. In some embodiments, the engineered human NK cells of the population, when co-cultured in vitro with a population of cancer cells, secrete IFNγ. In some embodiments, the ratio of engineered human NK cells to cancer cells is 0.1:1 to 2:1.

Provided herein includes a composition comprising a plurality of engineered cells described herein or any population of cells described herein. In some embodiments, a composition described herein is used in treating a subject in need thereof. In some embodiments, a composition described herein is used in treating cancer in a subject in need thereof. In some embodiments, the subject has multiple myeloma (MM), Hodgkin's lymphoma, lung cancer, leukemia, acute myeloid leukemia (AML), B-cell acute lymphoblastic leukemia (B-ALL), B-cell non-Hodgkin's lymphoma (B-NL), chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC) cancer, pancreatic cancer, melanoma, ovarian cancer, glioblastoma, liver cancer, colon cancer, or cervical cancer. In some embodiments, the subject is human.

Provided herein includes a method of obtaining cells for administration to a subject in need thereof, the method comprising: (a) obtaining or having obtained a plurality of any engineered cells described herein, and (b) maintaining the plurality of engineered cells for a time and under conditions sufficient for the cells to differentiate into lineage-restricted progenitor cells or fully differentiated somatic cells. Provided herein includes a method for treating of a subject in need thereof, the method comprising: (a) obtaining or having obtained a plurality of engineered cells described herein following differentiation into lineage-restricted progenitor cells or fully differentiated somatic cells; and (b) administering the lineage-restricted progenitor cells or fully differentiated somatic cells to the subject.

In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells are hematopoietic cells, pancreatic beta cells, epithelial cells, endodermal cells, macrophages, hepatocytes, adipocytes, kidney cells, blood cells, cardiomyocytes, or immune system cells.

In some embodiments, the subject has, is suspected of having, or is at risk for a cancer. In some embodiments, the subject has multiple myeloma (MM), Hodgkin's lymphoma, lung cancer, leukemia, acute myeloid leukemia (AML), B-cell acute lymphoblastic leukemia (B-ALL), B-cell non-Hodgkin's lymphoma (B-NL), chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC) cancer, pancreatic cancer, melanoma, ovarian cancer, glioblastoma, liver cancer, colon cancer, or cervical cancer. In some embodiments, the subject is human.

The present disclosure also provides an in vitro method for generating an engineered cell, the method comprising delivering to a cell (a) a first RNP complex comprising an RNA-guided nuclease and a gRNA targeting a target site in the B2M gene locus; (b) a first vector comprising a nucleic acid, the nucleic acid comprising: (i) nucleotide sequence encoding a SERPINB9 and a nucleotide sequence encoding an IL15/IL15Rα fusion; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); (c) a second RNP complex comprising an RNA-guided nuclease and a gRNA targeting a target site in the CIITA gene locus; and (d) a second vector comprising a nucleic acid, the nucleic acid comprising: (i) a nucleotide sequence encoding an anti-GPC3 CAR or an anti-GPR87 CAR and a nucleotide sequence encoding a HLA-E trimer; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); (e) a third RNP complex comprising an RNA-guided nuclease and a gRNA targeting a target site in the CISH gene locus; (f) a fourth RNP complex comprising an RNA-guided nuclease and a gRNA targeting a target site in the FAS gene locus; (g) a fifth RNP complex comprising an RNA-guided nuclease and a gRNA targeting a target site in the FLI1 gene locus; (h) a sixth RNP complex comprising an RNA-guided nuclease and a gRNA targeting a target site in the TGFBR1 or TGFβR2 gene locus, wherein any of (e)-(h) further comprises a third vector comprising a nucleotide sequence encoding CD16 or a fourth vector comprising a nucleotide sequence encoding CD64, wherein the sequence encoding CD16 or CD64 is flanked by homology arms having sequence homology with genomic sequence on either side of the site targeted by the gRNA of interest; (i) a seventh RNP complex comprising an RNA-guided nuclease and a gRNA targeting a target site in the CD38 gene locus; (j) an eighth RNP complex comprising an RNA-guided nuclease and a gRNA targeting a site in the B2M gene locus that differs from (a) and/or a fifth vector comprising a nucleotide sequence encoding an NKG2D CAR that is flanked by homology arms having sequence homology on either side of the site targeted by the gRNA of (j); wherein the B2M gene locus is cleaved at the target site and the nucleotide sequences encoding the SERPINB9 and the IL15/IL15Rα fusion are inserted into the B2M gene locus, thereby disrupting the B2M gene, wherein the CIITA gene locus is cleaved at the target site and the nucleotide sequences encoding the anti-GPC3 or anti-GPR87 CAR and the HLA-E trimer are inserted into the CIITA gene locus, thereby disrupting the CIITA gene, wherein the CISH gene locus is cleaved at the target site, thereby disrupting the CISH gene, wherein the FAS gene locus is cleaved at the target site, thereby disrupting the FAS gene, wherein the FLI1 gene is cleaved at the target site disrupting the FLI1 gene, wherein the TGFβR1 or TGFβR2 gene locus is cleaved at the target site, thereby disrupting the TGFβR1 or TGFβR2 gene, wherein the nucleotide sequences encoding CD16 and CD64 are inserted into the genome at the targeted sites, wherein the CD38 gene locus is cleaved at the target site, thereby disrupting the CD38 gene, and/or wherein the nucleotide sequence encoding NKG2D is inserted into a second site of the B2M gene.

In some embodiments, the gRNA targeting B2M comprises a spacer sequence corresponding to a sequence of SEQ ID NO: 34, SEQ ID NO: 78, or SEQ ID NO: 79. In some embodiments, the gRNA targeting CIITA comprises a spacer sequence corresponding to a sequence of SEQ ID NO: 13, SEQ ID NO: 14, SEQ ID NO: 15, SEQ ID NO: 16, or SEQ ID NO: 17. In some embodiments, the gRNA targeting ADAM17 comprises a spacer sequence corresponding to a sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In some embodiments, the gRNA targeting FAS comprises a spacer sequence corresponding to a sequence of SEQ ID NO: 35, SEQ ID NO: 37, SEQ ID NO: 38, SEQ ID NO: 39, SEQ ID NO: 53, SEQ ID NO: 55, or SEQ ID NO: 80. In some embodiments, the gRNA targeting CISH comprises a spacer sequence corresponding to a sequence of SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, or SEQ ID NO: 92. In some embodiments, the gRNA targeting REGNASE-1 comprises a spacer sequence corresponding to a sequence of SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, or SEQ ID NO: 101.

The first vector, the second vector, or both can be a plasmid vector. In some embodiments, the nucleotide sequence encoding the transgene is operably linked to an exogenous promoter (e.g., a CMV, EF1α, PGK, CAG, or UBC promoter).

In some embodiments, the nucleotide sequence encoding the HLA-E trimer sequence consists of SEQ ID NO: 75. In some embodiments, the nucleotide sequence encoding the IL15/IL15Rα sequence consists of SEQ ID NO: 76. In some embodiments, the SERPINB9-P2A-IL15/IL15Rα construct comprises a polynucleotide sequence of SEQ ID NO: 137.

In some embodiments, each RNP complex comprises a molar ratio of RNA-guided nuclease to gRNA of 1:3. In other embodiments, each RNP complex a molar ratio of RNA-guided nuclease to gRNA of 1:5. In some embodiments, the RNA-guided nuclease of each RNP complex is a Cas9 nuclease. In some embodiments, the Cas9 nuclease is linked to at least one nuclear localization signal.

The cell can be a stem cell (e.g., an embryonic stem cell, an adult stem cell, an induced pluripotent stem cell, or a hematopoietic stem cell). In some embodiments, the stem cell is a human stem cell.

In some embodiments, the nucleotide sequence having sequence homology with a genomic region located left of the target site in the B2M gene locus consists essentially of SEQ ID NO: 36, and the nucleotide having sequence homology with a genomic region located right of the target site in the B2M gene locus consists essentially of SEQ ID NO: 54. In some embodiments, wherein the nucleotide sequence having sequence homology with a genomic region located left of the target site in the CIITA gene locus consists essentially of SEQ ID NO: 22, and the nucleotide sequence having sequence homology with a genomic region located right of the target site in the CIITA gene locus consists essentially of SEQ ID NO: 32.

A plurality of engineered cells can be generated by any method described herein. In some embodiments, a plurality of engineered cells described herein are maintained for a time and under conditions sufficient for the cells to undergo differentiation. In certain embodiments, a plurality of engineered cells described herein are used in treating a subject in need thereof. In some embodiments, the subject is a human who has, is suspected of having, or is at risk for a cancer. In some embodiments, a subject is administered a plurality of engineered cells described herein.

Provided herein includes a method for treating of a subject in need thereof, the method comprising: (a) obtaining or having obtained the plurality of engineered cells described herein following differentiation into lineage-restricted progenitor cells or fully differentiated somatic cells; and (b) administering the lineage-restricted progenitor cells or fully differentiated somatic cells to the subject. Some embodiments provide a method of obtaining cells for administration to a subject in need thereof, the method comprising: (a) obtaining or having obtained the engineered cells described herein; and (b) maintaining the engineered cells for a time and under conditions sufficient for the cells to differentiate into lineage-restricted progenitor cells or fully differentiated somatic cells. The lineage-restricted progenitor cells can be hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells. In some embodiments, fully differentiated somatic cells are pancreatic beta cells, epithelial cells, endodermal cells, macrophages, hepatocytes, adipocytes, kidney cells, blood cells, cardiomyocytes, or immune system cells. In some embodiments, the subject is a human who has, is suspected of having, or is at risk for cancer. In some embodiments, the subject has multiple myeloma (MM), Hodgkin's lymphoma, lung cancer, leukemia, acute myeloid leukemia (AML), B-cell acute lymphoblastic leukemia (B-ALL), B-cell non-Hodgkin's lymphoma (B-NL), chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC), pancreatic cancer, melanoma, ovarian cancer, glioblastoma, liver cancer, colon cancer, or cervical cancer.

In some embodiments of the methods for generating NK cells from stem cells, the HSPCs express CD34. In some embodiments, the NK cells express CD56. In some embodiments, the NK cells express at least one activating receptor. In some embodiments, at least one activating receptor is selected from NKp44, NKp46, CD16, KIR2DL4, and any combination thereof. In some embodiments, the NK cells express at least one inhibitory receptor. In some embodiments, the at least one inhibitory receptor is selected from CD94, NKG2A, KIR3DL2, and any combination thereof. In some embodiments, the NK cells comprise at least one function associated with endogenous NK cells. In some embodiments, the at least one function comprises the ability to induce cell lysis and cell death of a target cell. In some embodiments, the at least one function comprises degranulation. In some embodiments, degranulation comprises release of perforin and granzyme B. In some embodiments, degranulation comprises expression of CD107a on the cell surface of an NK cell.

In some embodiments, the engineered cells comprise: a disrupted B2M gene; an insertion of a polynucleotide encoding SERPINB9 and a polynucleotide encoding a fusion of IL15 and IL15Rα (IL15/IL15Ra) in the disrupted B2M gene; a disrupted CIITA gene; and an insertion of a polynucleotide encoding a CAR and a polynucleotide encoding HLA-E in the disrupted CIITA gene, wherein the CAR is an anti-GPC3 CAR or an anti-GPR87 CAR; wherein the cell expresses SERPINB9, the IL15/IL15Rα fusion protein, HLA-E, and the CAR, and the cell has disrupted expressions of B2M and CIITA. In some embodiments, the engineered cell comprises a disrupted FAS gene, wherein the cell has disrupted expression of FAS. In some embodiments, the engineered cell comprises a disrupted CISH gene. The engineered cells can comprise: an insertion of a polynucleotide encoding CD64 in the disrupted CISH gene; and wherein the cell expresses CD64 and has disrupted expression of CISH.

In some embodiments, the engineered cells comprise: a disrupted B2M gene; an insertion of a polynucleotide encoding SERPINB9 and a polynucleotide encoding a fusion of IL15 and IL15Rα (IL15/IL15Ra) in the disrupted B2M gene; a disrupted CIITA gene; an insertion of a polynucleotide encoding an anti-GPR87 CAR and a polynucleotide encoding HLA-E in the disrupted CIITA gene; a disrupted CISH gene; and an insertion of a polynucleotide encoding CD64 in the disrupted CISH gene; wherein the cell expresses SERPINB9, the IL15/IL15Rα fusion protein, HLA-E, the anti-GPR87 CAR, and CD64, and wherein the cell has a disrupted expressions of B2M, CIITA, and CISH.

In some embodiments, the engineered cells comprise: a disrupted B2M gene; an insertion of a polynucleotide encoding SERPINB9 and a polynucleotide encoding a fusion of IL15 and IL15Rα (IL15/IL15Ra) in the disrupted B2M gene; a disrupted CIITA gene; an insertion of a polynucleotide encoding an anti-GPR87 CAR and a polynucleotide encoding HLA-E in the disrupted CIITA gene; a disrupted CISH gene; an insertion of a polynucleotide encoding CD64 in the disrupted CISH gene; and a disrupted FAS gene. In some embodiments, the cell expresses SERPINB9, the IL15/IL15Rα fusion protein, HLA-E, the anti-GPR87 CAR, and CD64. In some embodiments, the cell has disrupted expressions of B2M, CIITA, CISH, and FAS.

In some embodiments, the engineered cells comprise: a disrupted B2M gene; an insertion of a polynucleotide encoding SERPINB9 and a polynucleotide encoding a fusion of IL15 and IL15Rα (IL15/IL15Ra) in the disrupted B2M gene; a disrupted CIITA gene; an insertion of a polynucleotide encoding an anti-GPC3 CAR and a polynucleotide encoding HLA-E in the disrupted CIITA gene; a disrupted CISH gene; an insertion of a polynucleotide encoding CD64 in the disrupted CISH gene; and a disrupted FAS gene; wherein the cell expresses SERPINB9, the IL15/IL15Rα fusion protein, HLA-E, the anti-GPC3 CAR, and CD64, and wherein the cell has disrupted expressions of B2M, CIITA, CISH, and FAS. In some embodiments, the engineered cell comprises a disrupted TGFβR2 gene, wherein the cell has disrupted expression of TGFβR2.

In some embodiments, the polynucleotide encoding SERPINB9 comprises the sequence of SEQ ID NO: 129 and/or the polynucleotide encoding the IL15/IL15Rα fusion protein comprises the sequence of SEQ ID NO: 76. In some embodiments, the polynucleotide encoding the anti-GPC3 comprises the sequence of SEQ ID NO: 152 and/or anti-GPR87 CAR comprises the sequence of SEQ ID NO: 159. In some embodiments, the polynucleotide encoding HLA-E comprises the sequence of SEQ ID NO: 51. In some embodiments, the polynucleotide encoding HLA-E comprises a sequence encoding an HLA-E trimer comprising the sequence of SEQ ID NO: 75. In some embodiments, the polynucleotide encoding CD64 comprises the sequence of SEQ ID NO: 146.

The engineered cell can be a stem cell (e.g., an induced pluripotent stem cell (iPSC), a hematopoietic stem cell, an embryonic stem cell, or an adult stem cell). In some embodiments, the engineered cell is a genome-edited iPSC. In some embodiments, the engineered cell is a NK cell obtained from a genome-edited iPSC. In some embodiments, the engineered cell is a differentiated cell or a somatic cell. In some embodiments, the engineered cell is capable of being differentiated into lineage-restricted progenitor cells or fully differentiated somatic cells. In some embodiments, the engineered cell is a NK cell. In some embodiments, the NK cell has been differentiated from a genome-edited iPSC, wherein the NK cell comprises the genome edits of the genome-edited iPSC, and wherein the NK cell has not been genome-edited after the differentiation. In some embodiments, the engineered cell is capable of cell expansion in the absence of exogenous IL15 in cell culture media.

Disclosed herein include populations of cells comprising one or more engineered cells described herein. Also disclosed are populations of cells, comprising lineage-restricted progenitor cells or fully differentiated somatic cells derived from one or more engineered cells of any one of engineered cells of the disclosure.

In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells; and the fully differentiated somatic cells are pancreatic beta cells, epithelial cells, endodermal cells, macrophages, hepatocytes, adipocytes, kidney cells, blood cells, cardiomyocytes, or immune system cells. In some embodiments, the population of cells comprises NK cells, T cells, B cells, or NKT cells. In some embodiments, the population of cells comprises human NK cells. In some embodiments, the human NK cells express at least one, two, three, four or five of the markers of CD56, NKp44, NKp46, CD94, NKG2A, KIR2DL4, and a CAR, wherein the CAR is detected by Protein L binding. In some embodiments, the at least one, two, three, four or five markers are expressed in at least 25%, 30%, 40%, 50%, or 75% of the population of cells. In some embodiments, the population of cells comprising human NK cells has at least one of the following characteristics, or any combination thereof: (i) an alloimmune T cell reaction of less than 10% relative to a population of unmodified human NK cells, (ii) cytotoxic activity resulting in killing more than 50% of target cells when the population of cells comprising human NK cells are mixed with the target cells at the ratio of 1:1, and (iii) at least 50% increase in cellular viability relative to a population of unmodified human NK cells. In some embodiments, the population of cells comprising human NK cells has at least one of the following characteristics, or any combination thereof: (i) improved persistency, (ii) improved immune evasiveness, (iii) improved cytotoxic activity, (iv) improved antibody-dependent cellular cytotoxicity (ADCC) activity, and (v) improved anti-tumor activity; wherein the characteristics are improved relative to a population of unmodified human NK cells. In some embodiments, the population of cells comprising human NK cells, when co-cultured in vitro with a population of cancer cells, induce cell death of at least 60%, at least 70%, at least 80%, or at least 90% of the population of cancer cells after about 24 hours of co-culture. In some embodiments, the population of cells comprising human NK cells, when co-cultured in vitro with a population of cancer cells, secrete at least one of interferon gamma (IFNγ), tumor necrosis factor alpha (TNFα), and Granzyme B (GRNB). In some embodiments, the ratio of the human NK cells to cancer cells is 0.1:1 to 4:1.

Disclosed herein include compositions. In some embodiments, the compositions comprise the population of cells of any of the engineered cells described herein. In some embodiments, the composition can be used for treating a subject in need thereof. In some embodiments, the compositions can be used for treating cancer in a subject in need thereof. In some embodiments, the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC), pancreatic cancer, melanoma, ovarian cancer, glioblastoma, hepatocellular carcinoma, or cervical cancer. In some embodiments, the subject is human.

Disclosed herein include methods of obtaining cells for administration to a subject in need thereof, the method comprising: (a) obtaining or having obtained the population of cells as described herein, and (b) maintaining the population of cells for a time and under conditions sufficient for the one or more engineered cells to differentiate into lineage-restricted progenitor cells or fully differentiated somatic cells.

Disclosed herein include methods for treating a subject in need thereof. In some embodiments, the method comprises: (a) obtaining or having obtained the population of cells described herein; and (b) administering the lineage-restricted progenitor cells or fully differentiated somatic cells to the subject. In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells are pancreatic beta cells, epithelial cells, endodermal cells, macrophages, hepatocytes, adipocytes, kidney cells, blood cells, cardiomyocytes, or immune system cells. In some embodiments, the subject has, is suspected of having, or is at risk for a cancer. In some embodiments, the subject is human.

Disclosed herein include in vitro methods for generating an engineered cell. In some embodiments, the method comprising delivering to a cell: an RNP complex comprising an RNA-guided endonuclease and a gRNA targeting a target site in a B2M gene locus or an RNA-guided endonuclease and a gRNA targeting a target site in the B2M gene locus; a vector comprising a nucleic acid, the nucleic acid comprising: (i) a nucleotide sequence encoding SERPINB9 and a nucleotide sequence encoding a fusion of IL15 and IL15Rα (IL15/IL15Ra); (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); an RNP complex comprising an RNA-guided endonuclease and a gRNA targeting a target site in a CIITA gene locus or an RNA-guided endonuclease and a gRNA targeting a target site in the CIITA gene locus; and a vector comprising a nucleic acid, the nucleic acid comprising: (i) a nucleotide sequence encoding a CAR and a nucleotide sequence encoding an HLA-E trimer, wherein the CAR is an anti-GPC3 CAR or an anti-GPR87 CAR; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); wherein the B2M gene locus is cleaved at the target site and the nucleotide sequences encoding SERPINB9 and the IL15/IL15Rα fusion protein are inserted into the B2M gene locus, thereby disrupting the B2M gene, and wherein the CIITA gene locus is cleaved at the target site and the nucleotide sequences encoding the CAR and the HLA-E trimer are inserted into the CIITA gene locus, thereby disrupting the CIITA gene.

In some embodiments, the method comprises delivering to the cell: an RNP complex comprising an RNA-guided endonuclease and a gRNA targeting a target site in a CISH gene locus or an RNA-guided endonuclease and a gRNA targeting a target site in the CISH gene locus. In some embodiments, the method comprises delivering to the cell a vector comprising a nucleic acid, the nucleic acid comprising: (i) a nucleotide sequence encoding CD64; (ii) a nucleotide sequence having sequence homology with a genomic region located left of the target site in the CISH gene locus; and (iii) a nucleotide sequence having sequence homology with a genomic region located right of the target site in the CISH gene locus, wherein (i) is flanked by (ii) and (iii), wherein the CISH gene locus is cleaved at the target site and the nucleotide sequence encoding CD64 is inserted into the CISH gene locus, thereby disrupting the CISH gene. In some embodiments, the method comprises delivering to the cell: an RNP complex comprising an RNA-guided endonuclease and a gRNA targeting a target site in a FAS gene locus or an RNA-guided endonuclease and a gRNA targeting a target site in the FAS gene locus, wherein the FAS gene locus is cleaved at the target site, thereby disrupting the FAS gene. In some embodiments, the method comprises delivering to the cell: an RNP complex comprising an RNA-guided endonuclease and a gRNA targeting a target site in a TGFβR2 gene locus or an RNA-guided endonuclease and a gRNA targeting a target site in the TGFβR2 gene locus, wherein the TGFβR2 gene locus is cleaved at the target site, thereby disrupting the TGFβR2 gene.

In some embodiments, the RNP complex comprising the RNA-guided endonuclease and the gRNA targeting a target site in the CIITA gene locus or the RNA-guided endonuclease and the gRNA targeting a target site in the CIITA gene locus; and the vector comprising the nucleic acid comprising: (i) the nucleotide sequence encoding the CAR and the nucleotide sequence encoding the HLA-E trimer; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and (iii) are delivered to the cell after the nucleotide sequences encoding SERPINB9 and the IL15/IL15Rα fusion protein are inserted into the B2M gene locus. In some embodiments, the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii) are delivered to the cell after the nucleotide sequences encoding SERPINB9 and the IL15/IL15Rα fusion protein are inserted into the B2M gene locus and after the nucleotide sequences encoding the CAR and the HLA-E trimer are inserted into the CIITA gene locus. In some embodiments, the RNP complex comprising the RNA-guided endonuclease and the gRNA targeting the target site in the FAS gene locus or the RNA-guided endonuclease and the gRNA targeting the target site in the FAS gene locus are delivered to the cell after the nucleotide sequences encoding SERPINB9 and the IL15/IL15Rα fusion protein are inserted into the B2M gene locus, the nucleotide sequences encoding the CAR and the HLA-E trimer are inserted into the CIITA gene locus, and after the nucleotide sequence encoding CD64 is inserted into the CISH gene locus. In some embodiments, the RNP complex comprising the RNA-guided endonuclease and the gRNA targeting the target site in the TGFβR2 gene locus or the RNA-guided endonuclease and the gRNA targeting the target site in the TGFβR2 gene locus are delivered to the cell after the nucleotide sequences encoding SERPINB9 and the IL15/IL15Rα fusion protein are inserted into the B2M gene locus, after the nucleotide sequences encoding the CAR and the HLA-E trimer are inserted into the CIITA gene locus, and after the nucleotide sequence encoding CD64 is inserted into the CISH gene locus; and wherein the RNP complex comprising the RNA-guided endonuclease and the gRNA targeting a target site in the FAS gene locus or the RNA-guided endonuclease and the gRNA targeting a target site in the FAS gene locus is delivered to the cell after the nucleotide sequence encoding CD64 is inserted into the CISH gene locus, and after the TGFβR2 gene locus is disrupted.

In some embodiments, the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii) are delivered to the cell after the nucleotide sequences encoding SERPINB9 and the IL15/IL15Rα fusion protein are inserted into the B2M gene locus. In some embodiments, the RNP complex comprising the RNA-guided endonuclease and the gRNA targeting the target site in the FAS gene locus or the RNA-guided endonuclease and the gRNA targeting the target site in the FAS gene locus are delivered to the cell after the nucleotide sequences encoding SERPINB9 and the IL15/IL15Rα fusion protein are inserted into the B2M gene locus and after the nucleotide sequence encoding CD64 is inserted into the CISH gene locus. In some embodiments, the RNP complex comprising the RNA-guided endonuclease and the gRNA targeting the target site in the CIITA gene locus or the RNA-guided endonuclease and the gRNA targeting the target site in the CIITA gene locus and the vector comprising the nucleic acid comprising: (i) the nucleotide sequence encoding the CAR and the nucleotide sequence encoding the HLA-E trimer, wherein the CAR is an anti-GPC3 CAR or an anti-GPR87 CAR; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and (iii) are delivered to the cell after the nucleotide sequences encoding SERPINB9 and the IL15/IL15Rα fusion protein are inserted into the B2M gene locus, after the nucleotide sequence encoding CD64 is inserted into the CISH gene locus, and after the FAS gene locus is disrupted.

In some embodiments, the gRNA targeting the target site in the B2M gene locus comprises a spacer sequence of any one of SEQ ID NOs: 180 and 187-188. In some embodiments, the gRNA targeting the target site in the B2M gene locus comprises a spacer sequence of SEQ ID NO: 180. In some embodiments, the gRNA targeting the target site in the CIITA gene locus comprises a spacer sequence of any one of SEQ ID NOs: 175-179. In some embodiments, the gRNA targeting the target site in the CIITA gene locus comprises a spacer sequence of SEQ ID NO: 175. In some embodiments, the gRNA targeting the target site in the CISH gene locus comprises a spacer sequence of any one of SEQ ID NOs: 190-201. In some embodiments, the gRNA targeting the target site in the CISH gene locus comprises a spacer sequence of SEQ ID NO: 191. In some embodiments, the gRNA targeting the target site in the FAS gene locus comprises a spacer sequence of any one of SEQ ID NOs: 181-186 and 189. In some embodiments, the gRNA targeting the target site in the FAS gene locus comprises a spacer sequence of SEQ ID NO: 181 or SEQ ID NO: 182. In some embodiments, the gRNA targeting the target site in the TGFβR2 gene locus comprises a spacer sequence of SEQ ID NO: 211.

In some embodiments, the nucleotide sequence having sequence homology with a genomic region located left of the target site in the B2M gene locus comprises the sequence of SEQ ID NO: 36 and the nucleotide sequence having sequence homology with a genomic region located right of the target site in the B2M gene locus comprises the sequence of SEQ ID NO: 54. In some embodiments, the nucleotide sequence having sequence homology with a genomic region located left of the target site in the CIITA gene locus comprises the sequence of SEQ ID NO: 22 and the nucleotide sequence having sequence homology with a genomic region located right of the target site in the CIITA gene locus comprises the sequence of SEQ ID NO: 32. In some embodiments, the nucleotide sequence having sequence homology with a genomic region located left of the target site in the CISH gene locus comprises the sequence of SEQ ID NO: 145 and nucleotide sequence having sequence homology with a genomic region located right of the target site in the CISH gene locus comprises the sequence of SEQ ID NO: 148.

In some embodiments, the nucleotide sequence encoding SERPINB9 comprises the sequence of SEQ ID NO: 129 and the nucleotide sequence encoding the IL15/IL15Rα fusion protein comprises the sequence of SEQ ID NO: 76. In some embodiments, the nucleotide sequence encoding the anti-GPC3 CAR comprises the sequence of SEQ ID NO: 152. In some embodiments, the nucleotide sequence encoding the anti-GPR87 CAR comprises the sequence of SEQ ID NO: 159. In some embodiments, the nucleotide sequence encoding the HLA-E trimer comprises the sequence of SEQ ID NO: 75. In some embodiments, the nucleotide sequence encoding CD64 comprises the sequence of SEQ ID NO: 146. Disclosed herein include gRNAs or sgRNAs for targeting a TGFβR2 gene locus. In some embodiments, the gRNA or sgRNA comprises a spacer sequence of SEQ ID NO: 211.

The cell can be a stem cell (e.g., an embryonic stem cell, an adult stem cell, an induced pluripotent stem cell, or a hematopoietic stem cell). In some embodiments, the stem cell is a human stem cell. Disclosed herein include populations of cells comprising one or more engineered cells generated by the methods described herein. In some embodiments, the population of cells is maintained for a time and under conditions sufficient for at least a portion of the one or more engineered cells to undergo differentiation. The population of cells as obtained by the disclosed methods can be used in treating a subject in need thereof. In some embodiments, the subject is a human who has, is suspected of having, or is at risk for a cancer.

Disclosed herein include methods comprising administering to a subject in need thereof the population of cells generated by the disclosed methods. Disclosed herein include methods for treating a subject in need thereof. In some embodiments, the method comprises: (a) obtaining or having obtained the population of cells obtained by the methods disclosed herein, following differentiation into lineage-restricted progenitor cells or fully differentiated somatic cells; and (b) administering the lineage-restricted progenitor cells or fully differentiated somatic cells to the subject. Also provided herein are methods of obtaining cells for administration to a subject in need thereof, the method comprising: (a) obtaining or having obtained the population of cells described herein; and (b) maintaining the engineered cells for a time and under conditions sufficient for the one or more engineered cells to differentiate into lineage-restricted progenitor cells or fully differentiated somatic cells. In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, definitive endoderm, hepatoblasts, pancreatic endoderm progenitors, pancreatic endocrine progenitors, mesenchymal progenitor cells, muscle progenitor cells, blast cells, or neural progenitor cells; and the fully differentiated somatic cells are hepatocytes, pancreatic beta cells, epithelial cells, endodermal cells, macrophages, hepatocytes, adipocytes, kidney cells, blood cells, cardiomyocytes, or immune system cells. In some embodiments, the fully differentiated somatic cells are NK cells.

In some embodiments, the subject is a human who has, is suspected of having, or is at risk for a cancer. In some embodiments, the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, pancreatic cancer, liver cancer, melanoma, ovarian cancer, glioblastoma, or cervical cancer. In some embodiments, the liver cancer is hepatocellular carcinoma. In some embodiments, the lung cancer is non-small cell lung cancer.

In some embodiments, the method comprises administering the NK cells to the subject who has, is suspected of having or is at risk for a cancer, thereby inhibiting the progression of the cancer. Disclosed herein include methods of treating cancer. In some embodiments, the method comprises administrating NK cells obtained by the methods disclosed herein to a subject with cancer, thereby inhibiting progression of the cancer.

The method can comprise administering about 1×10² to about 1×10¹⁰ per gram of the engineered NK cells to the subject, e.g., about 1×10⁶ NK cells per gram to the subject. In some embodiments, the NK cells are administered to the subject more than once. In some embodiments, the NK cells are administered to the subject at least three times. In some embodiments, the NK cells are administered to the subject in a cycle of at least 7 days. In some embodiments, the NK cells are administered to the subject one, two, or three times in a week.

In some embodiments, inhibiting progression of the cancer comprises inhibition of growth of one or more tumors in the subject and/or reducing the number of cancer cells detected in the subject; relative to an untreated subject. In some embodiments, inhibiting progression of the cancer comprises inhibition of growth of one or more tumors in the subject and/or reducing the number of cancer cells detected in the subject; relative to the subject prior to administration of the NK cells. In some embodiments, the number of cancer cells detected in the subject increases by no more than 0.5-fold after administration of the NK cells, following one or more cycles of treatment. In some embodiments, the growth of at least one of the one or more tumors in the subject is inhibited by at least about 70%, about 75%, about 80%, about 85%, about 90%, about 95%, or about 100%, following one or more cycles of treatment. In some embodiments, the subject is tumor-free following one or more cycles of treatment.

In some embodiments, the NK cells persist in the subject for at least one week following administration. In some embodiments, the number of NK cells detected in the subject decreases by less than 25% one week after administration. In some embodiments, the number of NK cells detected in the subject decreases by less than 50% two weeks after administration. In some embodiments, the NK cells are localized to the site of the cancer following administration.

### BRIEF DESCRIPTION OF THE DRAWINGS

FIG. 1 provides a graph showing the cutting efficiency of 10 ADAM17 guides. Inducible pluripotent stem cells (iPSC) were electroporated with ADAM17 gRNA and sequenced to measured indel frequency.

FIG. 2 provides a graph showing the cutting efficiency of 5 CIITA guides. Human embryonic stem cells were electroporated with CIITA gRNA and sequenced to measured indel frequency.

FIG. 3 provides the plasmid map of BCMA CAR knock-in, and CIITA knock-out.

FIG. 4 provides the plasmid map of B2M-CAGGS-IL15-IR15 fusion-P2A-HLA-E. The IL15/IL15Ra-P2A-HLA-E trimer was inserted near exon 1 of the B2M gene locus to generate a B2M knock-out (KO)/IL15/IL15Ra-P2A-HLA-E knock-in (KI) plasmid.

FIG. 5A-FIG. 5B provide graphs of the flow cytometry analysis of HLA-E in IL15/IL15Ra-P2A-HLA-E trimer knock-in, B2M Null Human Pluripotent Stem Cells (hPSCs). Wild-type inducible pluripotent stem cells (iPSC) (FIG. 5A) and HLA-E edited iPSC (FIG. 5B) were analyzed using anti-HLA-E APC.

FIG. 6 demonstrates gating strategy for single-cell sorting of IL15/IL15Ra-P2A-HLA-E trimer knock-in, B2M Null hPSCs using an anti-HLA-E-PE antibody. FACS was used to sort single cells into 96-well plates.

FIG. 7A-FIG. 7B provide graphs of the flow cytometry analysis of IL-15 in single-cell “Clone 3” (IL15/IL15Ra-P2A-HLA-E trimer knock-in, B2M Null hPSCs). Wild-type inducible pluripotent stem cells (iPSC) (FIG. 7A) and Clone 3 IL-15 edited iPSC (FIG. 7B) were analyzed using anti-IL-15 PE.

FIG. 8 provides a line graph demonstrating cell growth in wild-type (WT) and Clone 3 (IL15/IL15Ra-P2A-HLA-E trimer knock-in, B2M Null hPSC) derived iNK cells when administered exogenous IL15 or not administered exogenous IL 15. Cells were administered 20 ng/mL of IL-15 in addition to SCF (20 ng/ml), Flt3L (15 ng/ml), IL-7 (20 ng/ml) on day 0 and day 4.

FIG. 9 provides a graph demonstrating K562 cell killing by WT and Clone 3 (IL15/IL15Ra-P2A-HLA-E trimer knock-in, B2M Null hPSC) derived iNK cells. Effector and K562 cells were plated at different effector: target (E:T) ratios for 24-hours. A no effector, K562 only cell, negative control was used.

FIG. 10 provides an image of an agarose gel demonstrating B2M indels. Clones with a band at 573 bp demonstrate a WT, unedited or heterozygous genotype. Clones with no band demonstrate a clone with successful knock-in.

FIG. 11 provides an image of an agarose gel demonstrating B2M zygosity results. A 2.5 kb band indicates a WT unedited clone. Clones with a 6.6 kb band indicate successful integration of the IL15/IL15Rα-P2A-HLA-E trimer.

FIG. 12 provides an image of an agarose gel demonstrating B2M knock-in genotyping results. No band indicates a WT unedited clone. A 1.1 kb band indicates successful integration of the IL15/IL15Rα-P2A-HLA-E trimer.

FIG. 13 provides an image of an agarose gel demonstrating CIITA genotyping results. A 557 bp indicates a WT unedited clone. Edited constructs do not have a band.

FIG. 14 provides an image of an agarose gel demonstrating CIITA zygosity results. results. A 2.5 kb band indicates a WT unedited clone. A 5.6 kb band indicates successful integration of the BCMA-CAR into the CIITA gene locus.

FIG. 15 provides an image of an agarose gel demonstrating CIITA genotyping results. The presence of a 1.5 kb band indicates successful integration of the KI construct into the CIITA gene locus, while the absence of a band indicates a WT genotype.

FIG. 16 provides histograms demonstrating pluripotency in hiPSC after genome editing. WT, Clone 1, and Clone 2 were stained for Oct4 and Sox2 and analyzed by flow cytometry.

FIG. 17 provides a graph demonstrating CD34/CD43 expression in Clone 1 (Line 1A c1), Clone 2 (Line 1A c2), Clone 3 (B2M-/HLA-E+/IL15+), a Line 1 clone, a CIITA-/BCMA CAR+ bulk population, and a ADAM17 KO clone (“Adam17-, c37”) cells compared to WT at Day 6 and Day 10 of differentiation from iPSC to iNK cells. Cells were analyzed by flow cytometry for CD34 and CD43 expression.

FIG. 18 provides a graph demonstrating CD45/CD56 expression in Clone 1 (Line 1A c1), Clone 2 (Line 1A c2), Clone 3 (B2M-/HLA-E+/IL15+), a Line 1 clone 2, a CIITA-/BCMA CAR+ bulk population, and a ADAM17 KO clone (“Adam17-, c37”) cells compared to WT at Day 10 and Day 14, Day 20, and Day 28 of differentiation from iPSC to iNK cells. Cells were analyzed by flow cytometry for CD45 and CD56 expression.

FIG. 19A-FIG. 19D provide graphs demonstrating expression of differentiation markers. FIG. 19A shows graphs of marker expression in Clone 1 (Line 1A c1), Clone 2 (Line 1A c2), Clone 3 (B2M-/HLA-E+/IL15+), a Line 1 clone 2, a CIITA-/BCMA CAR+ bulk population, and a ADAM17 KO clone (“Adam17-, c37”) cells compared to WT at Day 20 of differentiation from iPSC to iNK cells. Cells were analyzed by flow cytometry for CD56+/CD16+, CD56+/NKp44+, CD56+/NKp46+, CD56+/CD94+, and CD56+/NKG2A+ expression. FIG. 19B and FIG. 19C provide graphs demonstrating expression of differentiation markers in Clone 1 (Line 1A c1), Clone 2 (Line 1A c2), Clone 3 (B2M-/HLA-E+/IL15+), a Line 1 clone 2, a CIITA-/BCMA CAR+ bulk population, and a ADAM17 KO clone (“Adam17-, c37”) cells compared to WT at Day 28 (FIG. 19B) and Day 35 (FIG. 19C) of differentiation from iPSC to iNK cells. Cells were analyzed by flow cytometry for CD56+/CD16+, CD56+/NKp44+, CD56+/NKp46+, CD56+/CD94+, CD56+/NKG2A+, KIR2DL4, and KIR3DL2 expression. FIG. 19D provides a graph demonstrating expression of differentiation markers in Clone 1 compared to WT at Day 42 of differentiation from iPSC to iNK cells. Cells were analyzed by flow cytometry for CD56+/CD16+, CD56+/NKp44+, CD56+/NKp46+, CD56+/CD94+, CD56+/NKG2A+, and CD56+/CD57+ expression.

FIG. 20 provides a graph representing T-cell activation by differentiated iNK cells. Line 1A clone 1, Clone 3, and WT cells T cell activation was measured by carboxyfluorescein succinimidyl ester (CFSE) assay.

FIG. 21A-FIG. 21B provide graphs measuring K562 (FIG. 21A) and RPMI (FIG. 21B) cell killing by the indicated iNK cell line. WT, Line 1 clone 2, Line 1A Clone 1, Line 1A Clone 2, and CIITA-/BCMA CAR+ (“CIITA-/BCMA+”) bulk cells were cultured at different E:T ratios with K562 or RPMI cells for 24 hours.

FIG. 22 provides graphs measuring TNFa, IFNg, IL-7, and Granzyme B levels in WT and Line 1A clone 1 cells co-cultured at different E:T ratios with RPMI cells.

FIG. 23 provides flow cytometry graphs measuring Granzyme B and Perforin expressing cells at Day 14 (WT) and Day 36 (WT and Line 1A clones 1 and 2) of differentiation.

FIG. 24 provides graphs demonstrating cell count in wild-type (WT), Line 1A clone 1 (“Line 1A, c1”), Line 1A clone 2 (“Line 1A, c2”), and Clone 3 (“B2M-/HLA-E+/IL15/IL15Rα+”; IL15/IR15α-P2A-HLA-E trimer knock-in, B2M Null hPSC) derived iNK cells when administered exogenous IL15 or not administered exogenous IL15. Cells were administered SCF, Flt3L, IL7, and IL15 (“4”), SCF, Flt3L, and IL7 (“3/-IL15-”), no cytokines (“0”); or only IL15 (“IL15”) on day 0 and day 18.

FIG. 25A-FIG. 25B show CD31/CD34/CD45 expression profiles in aggregates after 10 days (FIG. 25A) or 14 days (FIG. 25B) of differentiation. Cell were differentiated from WT cells, IL15/IR15α-P2A-HLA-E trimer KI, BCMA CAR KI, CIITA Null, B2M Null, ADAM17 Null cells (“012.1”) cells, IL15/IR15α-P2A-HLA-E trimer KI, BCMA CAR KI, CIITA Null, B2M Null, ADAM17 Null, FAS Null, CISH Null, REGNASE-1 Null cells (“020.1”) cells, and IL15 KI, HLA-E KI, B2M null (“003.3”) cells.

FIG. 26 present CD45/CD56 expression profiles in aggregates after 10 days, 14 days, or 20 days of differentiation. Cell were differentiated from WT cells, IL15/IR15α-P2A-HLA-E trimer KI, BCMA CAR KI, CIITA Null, B2M Null, ADAM17 Null cells (“012.1”) cells, IL15/IR15α-P2A-HLA-E trimer KI, BCMA CAR KI, CIITA Null, B2M Null, ADAM17 Null, FAS Null, CISH Null, REGNASE-1 Null cells (“020.1”) cells, and IL15 KI, HLA-E KI, B2M null (“003.3”) cells.

FIG. 27A-FIG. 27B display exemplary data related to the killing activity of the NK cells of the disclosure. FIG. 27A shows the percent of killing of K562-GFP cells over 4 hours on Day 31 and FIG. 27B presents live NK cell ratios in NoTarget vs. 1:1 Killing in cells differentiated from WT cells, IL15/IR15α fusion-P2A-HLA-E KI into B2M and BCMA CAR into CIITA (“8.2”) cells, IL15/IR15α fusion-P2A-HLA-E KI into B2M, BCMA CAR into CIITA, and ADAM17 KO (“12.1”) cells, and IL15/IR15α fusion-P2A-HLA-E KI into B2M, BCMA CAR into CIITA, ADAM17 KO, FAS KO, CISH KO, and REGNASE-1 KO (“20.1”) cells. For each cell line, the no-target cell line control results are the first column, and the 1:1 data are shown in the second column.

FIG. 28A-FIG. 28B display exemplary data related to killing activity of iNK cells of the disclosure. FIG. 28A shows the percent of killing of MMIS-GFP cells over 4 hours on Day 31 and FIG. 28B presents live NK cell ratios in NoTarget vs. 1:1 Killing in cells differentiated from WT cells, IL15/IR15α fusion-P2A-HLA-E KI into B2M and BCMA CAR into CIITA (“8.2”) cells, IL15/IR15α fusion-P2A-HLA-E KI into B2M, BCMA CAR into CIITA, and ADAM17 KO (“12.1”) cells, and IL15/IR15α fusion-P2A-HLA-E KI into B2M, BCMA CAR into CIITA, ADAM17 KO, FAS KO, CISH KO, and REGNASE-1 KO (“20.1”) cells. For each cell line, the no-target cell line control results are the first column, and the 1:1 data are shown in the second column.

FIG. 29A-FIG. 29B display exemplary data related to killing activity of iNK cells of the disclosure. FIG. 29A shows killing of L428 cells after 4 hours and FIG. 29B shows killing of L428 cells after 24 hours by the indicated NK92 cells. For each iNK cell line, dilutions are shown from left to right for increasing E:T ratios (0.5:1, 1:1, 2:1).

FIG. 30A-FIG. 30B shows killing of KM-H2 cells. FIG. 30A shows killing of KM-H2 cells after 4 hours and FIG. 30B shows killing of KM-H2 cells after 24 hours by the indicated NK92 cells.

FIG. 31 presents the plasmid map of CD30 CAR 4-P2A-HLA-E trimer knock-in and CIITA knock-out.

FIG. 32 presents the plasmid map of CD30 CAR 5-P2A-HLA-E trimer knock-in and CIITA knock-out.

FIG. 33 presents the plasmid map of CD30 CAR 6-P2A-HLA-E trimer knock-in and CIITA knock-out.

FIG. 34 presents a map of the B2M-CAGGS-SERPINB9-P2A-HLA-E donor plasmid.

FIG. 35 shows FACS plots generated during the single cell sorting of the B2M-SERPINB9-P2A-HLA-E bulk population previously enriched by MACS.

FIG. 36 presents PCR analysis of SERPINB9/HLA-E KI at the B2M gene locus. The gel shows PCR amplification of B2M region of the genome with the 3′ primer stationed outside the knock-in (KI) site (not present in the plasmid donor) and the 5′ primer stationed inside the KI-only region. Presence of a 1.1 kilo base (kb) band indicates successful integration of the KI construct into the B2M gene locus, the absence of a band indicates a WT genotype.

FIG. 37 shows PCR 1 analysis of random plasmid insertions during knock-in of SERPINB9/HLA-E in the B2M gene locus. PCR was performed with 5′ and 3′ primers that bind outside of the homology arms within the KI plasmid. Presence of a 340 base pair (bp) band indicates that there is random integration of the plasmid backbone within the genome, clones without bands do not have random plasmid insertion.

FIG. 38 shows PCR 2 analysis of random plasmid insertions during knock-in of SERPINB9/HLA-E in the B2M gene locus. PCR was performed with 5′ and 3′ primers that bind outside of the homology arms within the KI plasmid. Presence of a 476 bp band indicates that there is random integration of the plasmid backbone within the genome, clones without bands do not have random plasmid insertion.

FIG. 39 shows zygosity at the B2M gene locus following knock-in of SERPINB9/HLA-E. Gel shows PCR products after amplification using primers spanning the gRNA cut site. Presence of a 573 bp band indicates a wildtype (WT) genotype which will be found in clones that are unedited or are heterozygous for the KI construct, a clone with a homozygous KI would not produce a band in this PCR because the KI size would be too large for the elongation time of this reaction.

FIG. 40 presents a time course of NK cell differentiation.

FIG. 41 shows the development of CD45+/CD56+ iNK over the differentiation time course, derived from WT or SERPINB9 KI/HLA-E KI/B2M KO clonal iPSCs.

FIG. 42A-FIG. 42E display exemplary data related to killing activity of iNK cells of the disclosure. FIG. 42A presents a plot of the percentage of target (iNK) cells killed by peripheral blood NK (PB-NK) cells from PBNK donor 4. Various iNK cells were incubated with PB-NK cells at various E:T ratios for 24 hours. FIG. 42B shows a plot of the percentage of target iNK cells killed by PB-NK cells from PBNK donor 6. Various iNK cells were incubated with PB-NK cells at various E:T ratios for 24 hours. FIG. 42C shows a plot of the percentage of target iNK cells killed by PB-NK cells from PBNK-CLL donor 1. Various iNK cells were incubated with PB-NK cells at various E:T ratios for 24 hours. FIG. 42D shows a plot of the percentage of target iNK cells killed by PB-NK cells from PBNK donor 4. Various iNK cells were incubated with PB-NK cells at various E:T ratios for 24 hours. FIG. 42E shows a plot of the percentage of target iNK cells killed by PB-NK cells from PBNK donor 6. Various iNK cells were incubated with PB-NK cells at various E:T ratios for 24 hours.

FIG. 43 presents a map the B2M-CAGGS-SERPINB9-P2A-IL15/IL15Rα fusion donor plasmid.

FIG. 44 shows percentage of cells in a bulk population that had HLA-ABC+ expression or IL15 surface expression. Cells were analyzed by flow cytometry.

FIG. 45A-FIG. 45B provide graphs demonstrating expression of differentiation markers in iPSC WT derived iNK cells with edited iPSC derived iNK cells (B2M KO/SERPINB9 KI/IL15/IL15Rα KI). Cells were analyzed by flow cytometry for CD56+/NKp44+, CD56+/NKp46+, CD56+/CD16+, CD56+ NKG2D+, CD56+/CD57+, and CD56+/CD33+ (FIG. 45A) and CD56+/NKG2A+, CD56+/FAS+, CD56+/FAS-L+, CD56+/KIR+, and PD-1+/TIGIT-(FIG. 45B).

FIG. 46 presents a map of the CISH-CAGGS-CD64-donor plasmid.

FIG. 47 presents a map of the CIITA-CAGGS-GPC3 CAR-P2A-HLA-E donor plasmid.

FIG. 48A-FIG. 48B display non-limiting exemplary cartoons related to use of the engineered iNK cells of the disclosure for treating hepatocellular carcinoma (FIG. 48A). In some embodiments, the therapeutic target of an iNK cell described herein is Glypican 3 (GPC3) as shown in FIG. 48B.

FIG. 49 displays non-limiting exemplary edits of the engineered iNK cells of the disclosure. In some embodiments, the cells comprise an insertion of one or more polynucleotides into the genome of the cell that can each express an IL15/IL115Ra fusion protein, an HLA-E trimer, SERPINB9 (CD64, and/or a CAR. In some embodiments, the cell can comprise one or more gene knockouts comprising B2M KO, CIITA KO, TGFβR2 KO, CISH KO, and/or FAS KO.

FIG. 50A-FIG. 50C display exemplary flow cytometry data for differentiated GPC3-CAR iNK cells. FIG. 50A displays flow cytometry data for GPC3 CAR. FIG. 50B-FIG. 50C display FACS analysis for markers of iNK differentiation in indicated engineered cell lines (L7V011-C5, L7V011-C7, L7V018-C2, L7V018-C3, L7V018-C6). From left to right in each graph: CD45+/CD56+, KIRs+/CD56+, KIR2DL4+/CD56+, NKp44+/CD56+, NKp46+/CD56+, NKG2D+/CD56+, NKG2A+/CD56+, CD16+/CD56+, CD15+.

FIG. 51A-FIG. 51D display non-limiting exemplary data related to the killing activity of anti-GPC3 CAR iNK cells disclosed herein. FIG. 51A shows a western blot of GPC3 levels in different cell lines. FIG. 51B displays flow cytometry data related to MHC class 1 expression of different cell lines. FIG. 51C-FIG. 51D display percent live target cells after 4 hrs (FIG. 51C) or 24 hrs (FIG. 51D) of co-culture with the indicated iNK cells.

FIG. 52A-FIG. 52C provide data related to cytokine release of engineered iNK cells of the disclosure following 24 hr of co-culture with indicated target cells: Granzyme B (GRNB, FIG. 52A), Interferon γ (IFNg, FIG. 52B), and tumor necrosis factor α (TNFα, FIG. 52C) levels are shown.

FIG. 53A-FIG. 53D provide data related to cytokine release of engineered iNK cells of the disclosure following 4 hr of co-culture with indicated target cells: Interferon γ (IFNg, FIG. 53A and FIG. 53C) and tumor necrosis factor α (TNFα, FIG. 53B and FIG. 53D) levels are shown.

FIG. 54 displays data showing that the iNK cells of the disclosure maintain potent killing activity following re-challenge.

FIG. 55 displays a chart of GPR87 expression levels across multiple cells lines retrieved from the Human Protein Atlas. Approximate location of data for indicated cell lines is shown.

FIG. 56A-FIG. 56C show graphs of data related to single cell release and characterization of anti-GPR87 CAR iNK cells of the disclosure. FIG. 56A displays single-cell count data. FIG. 56B displays data related to surface marker expression. For each marker pair, results from cell populations for F8, C2, C3, C5, C7, C8, and C10 cell lines are shown from left to right. FIG. 56C displays flow cytometry data related to Protein L expression for the indicated cell lines.

FIG. 57A-FIG. 57F display non-limiting exemplary data related to the killing activity of the engineered iNK cells targeting GPR87 of the disclosure. FIG. 57A displays killing of A549 cells by different iNK cell lines following 24 hrs of co-culture. FIG. 57B displays killing of PC-9 cells by different iNK cell lines following 24 hrs of co-culture.

FIG. 57C displays data of killing of A549 or PC-9 target cells by F8 cells following 4 or 24 hr of co-culture. FIG. 57D displays data of killing of A549 or PC-9 target cells by C5 cells following 4 or 24 hr of co-culture. FIG. 57E displays data of killing of A549 or PC-9 target cells by C7 cells following 4 or 24 hr of co-culture. FIG. 57F displays data of killing of A549 or PC-9 target cells by C10 cells following 4 or 24 hr of co-culture.

FIG. 58 depicts results of FACS sorting showing expression of IL-15 from cells in a population comprising cells with SerpinB9-IL-15/IL15Rα inserted into the B2M gene locus.

FIG. 59 depicts results of FACS sorting showing expression of CD64 from cells in a population comprising cells with CD64 coding sequence inserted into the CISH gene locus.

FIG. 60 depicts results of FACS sorting showing expression of HLA-E from cells in a population comprising cells with sequence encoding HLA-E and GPC3 is inserted into the CIITA gene locus.

FIG. 61A-FIG. 61C display data related to the in vivo efficacy of engineered iNK cells of the disclosure. FIG. 61A shows a non-limiting exemplary schematic of administration of iNK cells comprising anti-GPR87 CAR in NSG mice that were injected with PC-9 lung cancer cells. FIG. 61B-FIG. 61C shows quantification and images, respectively, of cancer cells in control mice (e.g., mice that were only injected with PC-9 cells) and in mice injected with both cancer cells and iNK cells.

FIG. 62 displays non-limiting exemplary data related to the biodistribution of iNK cells with IL15/IL15Rα fusion protein KI with or without FAS KO.

FIG. 63 displays non-limiting data of quantification of persistence of iNK cells in vivo. Time course data for each cell type (wild type iNK, IL15/IL15Rα fusion protein KI iNK, FAS KO; IL15/IL15Rα fusion protein KI iNK run 1, FAS KO; IL15/IL15Rα fusion protein KI iNK run 2) are shown from left to right.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

The present disclosure provides compositions of engineered stem cells (e.g., iPSCs), and lineage-restricted progenitor cells or fully differentiated somatic cells derived therefrom (e.g., NK cells, in particular, human NK cells).

In some embodiments, the engineered cells described herein evade immune response and/or survive following engraftment into a subject at higher success rates than an unmodified cell. In some embodiments, the engineered cells are hypoimmunogenic. In some embodiments, the engineered cells have improved persistency, (ii) improved immune evasiveness, (iii) improved cytotoxic activity, (iv) improved ADCC activity, and/or (v) improved anti-tumor activity as compared to a unmodified or wild-type cell, e.g., a wild-type iPSC or a wild-type NK cell.

In some embodiments, the engineered cells lack a functional major histocompatibility complex (MHC). In some embodiments, the engineered cells described herein are gene-edited to disrupt one or more of the genes of an MHC-I or MHC-II complex. In some embodiments, the engineered cells have a disrupted B2M gene and have a reduced expression of B2M (e.g., express less than 30%, less than 25%, less than 20%, less than 10%, less than 5% of the level of an unmodified cell) or do not express a detectable level of B2M. In some embodiments, the engineered cells have a disrupted CIITA gene and have a reduced expression of CIITA (e.g., express less than 30%, less than 25%, less than 20%, less than 10%, less than 5% of the level of an unmodified cell) or do not express a detectable level of CIITA. In some embodiments, the engineered cells have a disrupted ADAM17 gene and have a reduced expression of ADAM17 (e.g., express less than 30%, less than 25%, less than 20%, less than 10%, less than 5% of the level of an unmodified cell) or do not express a detectable level of ADAM17. In some embodiments, the engineered cells have a disrupted FAS gene and have a reduced expression of FAS (e.g., express less than 30%, less than 25%, less than 20%, less than 10%, less than 5% of the level of an unmodified cell) or do not express a detectable level of FAS. In some embodiments, the engineered cells have a disrupted CISH gene and have a reduced expression of CISH (e.g., express less than 30%, less than 25%, less than 20%, less than 10%, less than 5% of the level of an unmodified cell) or do not express a detectable level of CISH. In some embodiments, the engineered cells have a disrupted CD38 gene and have a reduced expression of CD38 (e.g., express less than 30%, less than 25%, less than 20%, less than 10%, less than 5% of the level of an unmodified cell) or do not express a detectable level of CD38. In some embodiments, the engineered cells have a disrupted FLI1 gene and have a reduced expression of FLI1 (e.g., express less than 30%, less than 25%, less than 20%, less than 10%, less than 5% of the level of an unmodified cell) or do not express a detectable level of FLI1. In some embodiments, the engineered cells have a disrupted TGFβR1 gene and have a reduced expression of TGFβR1 (e.g., express less than 30%, less than 25%, less than 20%, less than 10%, less than 5% of the level of an unmodified cell) or do not express a detectable level of TGFβR1. In some embodiments, the engineered cells have a disrupted TGFβR2 gene and have a reduced expression of TGFβR2 (e.g., express less than 30%, less than 25%, less than 20%, less than 10%, less than 5% of the level of an unmodified cell) or do not express a detectable level of TGFβR2. In some embodiments, the engineered cells have a disrupted ZEB1 gene and have a reduced expression of ZEB1 (e.g., express less than 30%, less than 25%, less than 20%, less than 10%, less than 5% of the level of an unmodified cell) or do not express a detectable level of ZEB1.

In some embodiments, the engineered cells have a disrupted PD-1 gene and have a reduced expression of PD-1 (e.g., express less than 30%, less than 25%, less than 20%, less than 10%, less than 5% of the level of an unmodified cell) or do not express a detectable level of PD-1.

In some embodiments, the engineered cells have a disrupted TIGIT gene and have a reduced expression of TIGIT (e.g., express less than 30%, less than 25%, less than 20%, less than 10%, less than 5% of the level of an unmodified cell) or do not express a detectable level of TIGIT. In some embodiments, the engineered cells have a disrupted REGNASE1 gene and have a reduced expression of REGNASE-1 (e.g., express less than 30%, less than 25%, less than 20%, less than 10%, less than 5% of the level of an unmodified cell) or do not express a detectable level of REGNASE-1. In some embodiments, the engineered cells have a disrupted NKG2A gene and have a reduced expression of NKG2A (e.g., express less than 30%, less than 25%, less than 20%, less than 10%, less than 5% of the level of an unmodified cell) or do not express a detectable level of NKG2A. In some embodiments, the engineered cells have a disrupted ADORA2A/ADORA2B gene and have a reduced expression of A2a/A2b (e.g., express less than 30%, less than 25%, less than 20%, less than 10%, less than 5% of the level of an unmodified cell) or do not express a detectable level of A2a/A2b.

In some embodiments, the genome of the engineered cells has a disrupted B2M gene and one or more inserted polynucleotide(s) encoding one or all of: SERPINB9, IL15, IL15Rα, and HLA-E (e.g., (i) a polynucleotide encoding a fusion protein of IL15 and IL15Rα and an HLA-E trimer comprising a B2M signal peptide fused to an HLA-G presentation peptide fused to the B2M membrane protein fused to the HLA-E protein without a signal peptide, or (ii) a polynucleotide encoding a SERPINB9 and fusion protein of IL15 and IL15Rα). The inserted polynucleotide(s) can be inserted in the disrupted B2M gene locus (e.g., in exon 1 of the B2M gene locus). In some embodiments, the cell further comprises a polynucleotide encoding NKG2D CAR inserted into a second location of the disrupted B2M gene.

In some embodiments, the genome of the engineered cells has a disrupted CIITA gene and one or more inserted polynucleotide(s) encoding one or more CARs (e.g., an anti-GPC3 CAR, an anti-GPR87 CAR, an anti-BCMA CAR, an anti-CD30 CAR, an anti-CD19-CD20-BCMA CAR, an anti-A33 CAR, or an anti-CD33 CAR) and HLA-E (e.g., an HLA-E trimer comprising a B2M signal peptide fused to an HLA-G presentation peptide fused to the B2M membrane protein fused to the HLA-E protein without a signal peptide). The inserted polynucleotide(s) can be inserted in the disrupted CIITA gene locus (e.g., in exon 2 of the CIITA gene locus).

In some embodiments, the genome of the engineered cells has one or more inserted polynucleotide(s) encoding CD16 and/or CD64. The polynucleotides encoding CD16 can be inserted in the disrupted CISH gene, the disrupted FAS gene, the disrupted FLI1 gene, the disrupted TGFβR1 gene, and/or the disrupted TGFβR2 gene. The polynucleotides encoding CD64 can be inserted in the disrupted CISH gene, the disrupted FAS gene, the disrupted FLI1 gene, or the disrupted TGFβR1 gene, and/or the disrupted TGFβR2 gene.

In some embodiments, the engineered cells described herein are stem cells. In some embodiments, the engineered cells described herein are iPSCs. In some embodiments, the engineered cells described herein are mesodermal cells. In some embodiments, the engineered cells described herein are hemogenic endothelium (HE) cells (e.g., definitive hemogenic endothelium cells). In some embodiments, the engineered cells described herein are hematopoietic stem or progenitor cells (HSPCs) (e.g., definitive hematopoietic stem or progenitor cells). In some embodiments, the engineered cells described herein are common lymphoid progenitor (CLP) cells. In some embodiments, the engineered cells described herein are NK progenitor cells. In some embodiments, the engineered cells described herein are immature NK cells. In some embodiments, the engineered cells described herein are NK cells. In some embodiments, the engineered cells described herein are fully differentiated cells (e.g., NK cells). In some embodiments, stem cells (e.g., iPSCs) are gene-edited as described herein and then differentiated into one, two, three, four, five, six or more of the following cell types: mesodermal cells, HE cells, HSPCs, CLP cells, NK progenitor cells, immature NK cells and NK cells. In some embodiments, the differentiated cells maintain all edits made in the cells from which they were derived (e.g., NK cells maintain all edits of gene-edited stem cells (e.g., iPSCs) from which they were derived. In some embodiments, the engineered cells described herein are CD34+ cells. In some embodiments, the engineered cells described herein are multipotent progenitors (MPP). In some embodiments, the engineered cells described herein are common lymphoid progenitor cells. In some embodiments, the engineered cells described herein are T cell progenitors. In some embodiments, a cell such as an NK cell (derived from an engineered stem cell) comprises the gene-edits described herein.

In some embodiments, the engineered cells comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; and an insertion of a polynucleotide encoding a CAR and a polynucleotide encoding HLA-E in the disrupted CIITA gene, wherein the CAR is an anti-GPC3 CAR or an anti-GPR87 CAR; wherein the cell expresses SERPINB9, the IL15/IL15Rα fusion protein, HLA-E, and the CAR, and the cell has disrupted expressions of B2M and CIITA. In some embodiments, the engineered cell comprises a disrupted FAS gene, wherein the cell has disrupted expression of FAS. In some embodiments, the engineered cell comprises a disrupted CISH gene. The engineered cells can comprise: an insertion of a polynucleotide encoding CD64 in the disrupted CISH gene; and wherein the cell expresses CD64 and has disrupted expression of CISH.

The engineered cells can comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; an insertion of a polynucleotide encoding an anti-GPR87 CAR and a polynucleotide encoding HLA-E in the disrupted CIITA gene; a disrupted CISH gene; and an insertion of a polynucleotide encoding CD64 in the disrupted CISH gene; wherein the cell expresses SERPINB9, the IL15/IL15Rα fusion protein, HLA-E, the anti-GPR87 CAR, and CD64, and wherein the cell has a disrupted expressions of B2M, CIITA, and CISH.

In some embodiments, the engineered cells comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; an insertion of a polynucleotide encoding an anti-GPC3 CAR and a polynucleotide encoding HLA-E in the disrupted CIITA gene; a disrupted CISH gene; an insertion of a polynucleotide encoding CD64 in the disrupted CISH gene; and a disrupted FAS gene; wherein the cell expresses SERPINB9, the IL15/IL15Rα fusion protein, HLA-E, the anti-GPC3 CAR, and CD64, and wherein the cell has disrupted expressions of B2M, CIITA, CISH, and FAS.

Disclosed herein include populations of cells comprising one or more engineered cells described herein. Also disclosed are populations of cells, comprising lineage-restricted progenitor cells or fully differentiated somatic cells derived from one or more engineered cells of any one of engineered cells of the disclosure. Disclosed herein include compositions. In some embodiments, the compositions comprise the population of cells of any of the engineered cells described herein.

Disclosed herein include methods for treating a subject in need thereof. In some embodiments, the method comprises: (a) obtaining or having obtained the population of cells described herein; and (b) administering the lineage-restricted progenitor cells or fully differentiated somatic cells to the subject.

Disclosed herein include in vitro methods for generating an engineered cell. In some embodiments, the method comprising delivering to a cell: an RNP complex comprising an RNA-guided endonuclease and a gRNA targeting a target site in a B2M gene locus or an RNA-guided endonuclease and a gRNA targeting a target site in the B2M gene locus; a vector comprising a nucleic acid, the nucleic acid comprising: (i) a nucleotide sequence encoding SERPINB9 and a nucleotide sequence encoding a fusion of IL15 and IL15Rα (IL15/IL15Rα); (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); an RNP complex comprising an RNA-guided endonuclease and a gRNA targeting a target site in a CIITA gene locus or an RNA-guided endonuclease and a gRNA targeting a target site in the CIITA gene locus; and a vector comprising a nucleic acid, the nucleic acid comprising: (i) a nucleotide sequence encoding a CAR and a nucleotide sequence encoding an HLA-E trimer, wherein the CAR is an anti-GPC3 CAR or an anti-GPR87 CAR; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); wherein the B2M gene locus is cleaved at the target site and the nucleotide sequences encoding SERPINB9 and the IL15/IL15Rα fusion protein are inserted into the B2M gene locus, thereby disrupting the B2M gene, and wherein the CIITA gene locus is cleaved at the target site and the nucleotide sequences encoding the CAR and the HLA-E trimer are inserted into the CIITA gene locus, thereby disrupting the CIITA gene.

Disclosed herein include methods comprising administering to a subject in need thereof the population of cells generated by the disclosed methods. Disclosed herein include methods for treating a subject in need thereof. In some embodiments, the method comprises: (a) obtaining or having obtained the population of cells obtained by the methods disclosed herein, following differentiation into lineage-restricted progenitor cells or fully differentiated somatic cells; and (b) administering the lineage-restricted progenitor cells or fully differentiated somatic cells to the subject. Also provided herein are methods of obtaining cells for administration to a subject in need thereof, the method comprising: (a) obtaining or having obtained the population of cells described herein; and (b) maintaining the engineered cells for a time and under conditions sufficient for the one or more engineered cells to differentiate into lineage-restricted progenitor cells or fully differentiated somatic cells. Disclosed herein include gRNAs or sgRNAs for targeting a TGFβR2 gene locus. In some embodiments, the gRNA or sgRNA comprises a spacer sequence of SEQ ID NO: 211.

Disclosed herein include methods of treating cancer. In some embodiments, the method comprises administrating NK cells obtained by the methods disclosed herein to a subject with cancer, thereby inhibiting progression of the cancer.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34+ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3-and CD56+, expressing at least one of NKG2C and CD57, and optionally, CD16, but lack expression of one or more of the following: PLZF, SYK, FceRy, and EAT-2. In some embodiments, isolated subpopulations of CD56+ NK cells comprise expression of CD16, NKG2C, CD57, NKG2D, NCR ligands, NKp30, NKp40, NKp46, activating and inhibitory KIRs, NKG2A and/or DNAM-1.

As used herein, the terms “disruption,” “genetic modification” or “gene-edit” generally refer to a genetic modification wherein a site or region of genomic DNA is altered, e.g., by a deletion or insertion, by any molecular biology method, e.g., methods described herein, e.g., by delivering to a site of genomic DNA an endonuclease and at least one gRNA. Exemplary genetic modifications include insertions, deletions, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product. As used herein, the term “engineered cell” refers to a cell with any disruption, genetic modification or gene-edit.

As used herein, the term “deletion” which may be used interchangeably with the terms “genetic deletion”, “knock-out”, or “KO”, generally refers to a genetic modification wherein a site or region of genomic DNA is removed by any molecular biology method, e.g., methods described herein, e.g., by delivering to a site of genomic DNA an endonuclease and at least one gRNA. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of an entire target gene, e.g., a B2M gene, a CIITA gene, a ADAM17 gene, a FAS gene, a CISH gene, a CD38 gene, a FLI1 gene, a TGFβR1 gene, a TGFβR2 gene, a ZEB1 gene, a PD-1 gene, a TIGIT gene, a NKG2A gene, an ADORA2A/ADORA2B gene, and/or a REGNASE-1 gene. In some embodiments, a deletion involves the removal of part of a target gene, e.g., all or part of a promoter and/or coding sequence of a B2M gene, a CIITA gene, a ADAM17 gene, a FAS gene, a CISH gene, a CD38 gene, a FLI1 gene, a TGFβR1 gene, a TGFβR2 gene, a ZEB1 gene, a PD-l gene, a TIGIT gene, a NKG2A gene, an ADORA2A/ADORA2B gene, and/or a REGNASE-1 gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR-associated endonuclease. In some embodiments, an endonuclease is a RNA-guided endonuclease. In some aspects, the RNA-guided endonuclease is a CRISPR nuclease, e.g., a Type II CRISPR Cas9 endonuclease or a Type V CRISPR Cpf1 endonuclease. In some embodiments, an endonuclease is a Cas1, Cas1B, Cas2, Cas3, Cas4, Cas5, Cas6, Cas7, Cas8, Cas9 (also known as Csnl and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or combinations thereof. In some embodiments, an endonuclease may introduce one or more single-stranded breaks (SSBs) and/or one or more double-stranded breaks (DSBs).

As used herein, the term “guide RNA” or “gRNA” generally refers to short ribonucleic acid that can interact with, e.g., bind to, to an endonuclease and bind, or hybridize to a target genomic site or region. In some embodiments, a gRNA is a single-molecule guide RNA (sgRNA). In some embodiments, a gRNA may comprise a spacer extension region. In some embodiments, a gRNA may comprise a tracrRNA extension region. In some embodiments, a gRNA is single-stranded. In some embodiments, a gRNA comprises naturally occurring nucleotides. In some embodiments, a gRNA is a chemically modified gRNA. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, a gRNA may be pre-complexed with a DNA endonuclease.

As used herein, the term “insertion” which may be used interchangeably with the terms “genetic insertion” or “knock-in”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion involves the introduction of a polynucleotide that encodes a protein of interest. In some embodiments, an insertion involves the introduction of a polynucleotide that encodes a tolerogenic factor (e.g., HLA-E trimer), a CAR (e.g., against GPC3, GPR87, NKG2D, CD19, CD20, CD30, CD33, A33, BCMA, and/or combination thereof), a fusion protein of IL15 and ILRa, CD16, CD64, and/or SERPINB9. In some embodiments, an insertion involves the introduction of an exogenous promoter, e.g., a constitutive promoter, e.g., a CAG promoter. In some embodiments, an insertion involves the introduction of a polynucleotide that encodes a noncoding gene. In general, a polynucleotide to be inserted is flanked by sequences (e.g., homology arms) having substantial sequence homology with genomic DNA at or near the site of insertion.

As used herein, the terms “Major histocompatibility complex class I” or “MHC-I” generally refer to a class of biomolecules that are found on the cell surface of all nucleated cells in vertebrates, including mammals, e.g., humans; and function to display peptides of non-self or foreign antigens, e.g., proteins, from within the cell (i.e. cytosolic) to cytotoxic T cells, e.g., CD8+ T cells, in order to stimulate an immune response. In some embodiments, a MHC-I biomolecule is a MHC-I gene or a MHC-I protein. Complexation of MHC-I proteins with beta-2 microglobulin (B2M) protein is required for the cell surface expression of all MHC-I proteins. In some embodiments, decreasing the expression of a MHC-I human leukocyte antigen (HLA) relative to an unmodified cell involves a decrease (or reduction) in the expression of a MHC-I gene. In some embodiments, decreasing the expression of a MHC-I human leukocyte antigen (HLA) relative to an unmodified cell involves a decrease (or reduction) in the cell surface expression of a MHC-I protein. In some embodiments, a MHC-I biomolecule is HLA-A (NCBI Gene ID No: 3105), HLA-B (NCBI Gene ID No: 3106), HLA-C(NCBI Gene ID No: 3107), or B2M (NCBI Gene ID No: 567).

As used herein, the term “Major histocompatibility complex class II” or “MHC-II” generally refer to a class of biomolecules that are typically found on the cell surface of antigen-presenting cells in vertebrates, including mammals, e.g., humans; and function to display peptides of non-self or foreign antigens, e.g., proteins, from outside of the cell (extracellular) to cytotoxic T cells, e.g., CD8+ T cells, in order to stimulate an immune response. In some embodiments, an antigen-presenting cell is a dendritic cell, macrophage, or a B cell. In some embodiments, a MHC-II biomolecule is a MHC-II gene or a MHC-II protein. In some embodiments, decreasing the expression of a MHC-II human leukocyte antigen (HLA) relative to an unmodified cell involves a decrease (or reduction) in the expression of a MHC-II gene. In some embodiments, decreasing the expression of a MHC-II human leukocyte antigen (HLA) relative to an unmodified cell involves a decrease (or reduction) in the cell surface expression of a MHC-II protein. In some embodiments, a MHC-II biomolecule is HLA-DPA (NCBI Gene ID No: 3113), HLA-DPB (NCBI Gene ID No: 3115), HLA-DMA (NCBI Gene ID No: 3108), HLA-DMB (NCBI Gene ID No: 3109), HLA-DOA (NCBI Gene ID No: 3111), HLA-DOB (NCBI Gene ID No: 3112), HLA-DQA (NCBI Gene ID No: 3117), HLA-DQB (NCBI Gene ID No: 3119), HLA-DRA (NCBI Gene ID No: 3122), or HLA-DRB (NCBI Gene ID No: 3123).

As used herein, the term “polynucleotide”, which may be used interchangeably with the term “nucleic acid” generally refers to a biomolecule that comprises two or more nucleotides. In some embodiments, a polynucleotide comprises at least two, at least five at least ten, at least twenty, at least 30, at least 40, at least 50, at least 100, at least 200, at least 250, at least 500, or any number of nucleotides. A polynucleotide may be a DNA or RNA molecule or a hybrid DNA/RNA molecule. A polynucleotide may be single-stranded or double-stranded. In some embodiments, a polynucleotide is a site or region of genomic DNA. In some embodiments, a polynucleotide is an endogenous gene that is comprised within the genome of an unmodified cell or gene-edited iPSC. In some embodiments, a polynucleotide is an exogenous polynucleotide that is not integrated into genomic DNA. In some embodiments, a polynucleotide is an exogenous polynucleotide that is integrated into genomic DNA. In some embodiments, a polynucleotide is a plasmid or an adeno-associated viral vector. In some embodiments, a polynucleotide is a circular or linear molecule.

As used herein, the term “subject” refers to a mammal. In some embodiments, a subject is non-human primate or rodent. In some embodiments, a subject is a human. In some embodiments, a subject has, is suspected of having, or is at risk for, a disease or disorder. In some embodiments, a subject has one or more symptoms of a disease or disorder.

As used herein, the term “transcriptional regulator of MHC-I or MHC-II” generally refers to a biomolecule that modulates, e.g., increases or decreases, the expression of a MHC-I and/or MHC-II human leukocyte antigen. In some embodiments, a biomolecule is a polynucleotide, e.g., a gene, or a protein. In some embodiments, a transcriptional regulator of MHC-I or MHC-II will increase or decrease the cell surface expression of at least one MHC-I or MHC-II protein. In some embodiments, a transcriptional regulator of MHC-I or MHC-II will increase or decrease the expression of at least one MHC-I or MHC-II gene. In some embodiments, the transcriptional regulator is CIITA (NCBI Gene ID No: 4261) or NLRC5 (NCBI Gene ID No: 84166). In some embodiments, deletion or reduction of expression of CIITA or NLRC5 decreases expression of at least one MHC-I or MHC-II gene.

As used herein, the term “engineered cell” generally refers to a genetically modified cell that is less susceptible to allogeneic rejection during a cellular transplant and/or demonstrates increased survival after transplantation, relative to an unmodified cell. In some embodiments, a genetically modified cell as described herein is an engineered cell. In some embodiments, the engineered cell has increased immune evasion and/or cell survival compared to an unmodified cell. In some embodiments, the engineered cell has increased cell survival compared to an unmodified cell. In some embodiments, the engineered cell has improved persistency, (ii) improved immune evasiveness, (iii) improved cytotoxic activity, (iv) improved ADCC activity, and/or (v) improved anti-tumor activity compared to an unmodified cell. In some embodiments, an engineered cell may be a stem cell. In some embodiments, an engineered cell may be an embryonic stem cell (ESC), an adult stem cell (ASC), an induced pluripotent stem cell (iPSC), or a hematopoietic stem or progenitor cell (HSPC). In some embodiments, an engineered cell may be a differentiated cell. In some embodiments, an engineered cell may be a somatic cell (e.g., immune system cells). In some embodiments, an engineered cell is administered to a subject. In some embodiments, an engineered cell is administered to a subject who has, is suspected of having, or is at risk for a disease. In some embodiments, the engineered cell is capable of being differentiated into lineage-restricted progenitor cells or fully differentiated somatic cells. In some embodiments, the lineage-restricted progenitor cells are pancreatic endoderm progenitors, pancreatic endocrine progenitors, mesenchymal progenitor cells, muscle progenitor cells, blast cells, or neural progenitor cells. In some embodiments, the fully differentiated somatic cells are endocrine secretory cells such as pancreatic beta cells, epithelial cells, endodermal cells, macrophages, hepatocytes, adipocytes, kidney cells, blood cells, or immune system cells.

As used herein, the term “unmodified cell” refers to a cell that has not been subjected to a genetic modification involving a polynucleotide or gene that encodes any of the genes described herein. In some embodiments, an unmodified cell may be a stem cell. In some embodiments, an unmodified cell may be an embryonic stem cell (ESC), an adult stem cell (ASC), an induced pluripotent stem cell (iPSC), or a hematopoietic stem or progenitor cell (HSPC). In some embodiments, an unmodified cell may be a differentiated cell. In some embodiments, an unmodified cell may be selected from somatic cells (e.g., immune system cells, e.g., a T cell, e.g., a CD8+ T cell). If a gene-edited iPSC or NK cell is compared “relative to an unmodified cell”, the iPSC or NK cell and the unmodified cell are the same cell type or share a common parent cell line, e.g., a gene-edited NK cell is compared relative to an unmodified NK cell.

As used herein, the term “within or near a gene” refers to a site or region of genomic DNA that is an intronic or exonic component of a said gene or is located proximal to a said gene. In some embodiments, a site of genomic DNA is within a gene if it comprises at least a portion of an intron or exon of said gene. In some embodiments, a site of genomic DNA located near a gene may be at the 5′ or 3′ end of said gene (e.g., the 5′ or 3′ end of the coding region of said gene). In some embodiments, a site of genomic DNA located near a gene may be a promoter region or repressor region that modulates the expression of said gene. In some embodiments, a site of genomic DNA located near a gene may be on the same chromosome as said gene. In some embodiments, a site or region of genomic DNA is near a gene if it is within 50 Kb, 40 Kb, 30 Kb, 20 Kb, 10 Kb, 5 Kb, 1 Kb, or closer to the 5′ or 3′ end of said gene (e.g., the 5′ or 3′ end of the coding region of said gene).

As used herein, the term “tolerogenic factor” generally refers to a protein (e.g., expressed by a polynucleotide as described herein) that, when increased or decreased in a cell, enables the cell, e.g., an engineered cell, to inhibit or evade immune rejection after transplantation or engraftment into a host subject at higher rates relative to an unmodified cell. In some embodiments, a tolerogenic factor is a human tolerogenic factor. In some embodiments, the genetic modification of at least one tolerogenic factor (e.g., the insertion or deletion of at least one tolerogenic factor) enables a cell, e.g., an engineered cell. to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, a tolerogenic factor is HLA-E (NCBI Gene ID No: 3133), HLA-G (NCBI Gene ID No: 3135), CTLA-4 (NCBI Gene ID No: 1493), CD47 (NCBI Gene ID No: 961), or PD-L1 (NCBI Gene ID No: 29126). In some embodiments, a tolerogenic factor is inserted into a cell, e.g., an engineered cell. In some embodiments, a tolerogenic factor is deleted from a cell, e.g., an engineered cell. In some embodiments, an insertion of a polynucleotide that encodes HLA-E, HLA-G, CTLA-4, CD47, and/or PD-L1 enables a cell, e.g., an engineered cell, to inhibit or evade immune rejection after transplantation or engraftment into a host subject.

### Gene Editing of Engineered Cells

Described herein are strategies to enable genetically modified cells to evade immune response and/or increase their survival, or viability following engraftment into a subject. In some embodiments, these strategies enable gene-edited cells to evade immune response and/or survive at higher success rates than an unmodified cell.

In some embodiments, any cells described herein are gene-edited using any of the gene-editing methods described herein (e.g., using CRISPR/Cas gene editing to insert or delete one or more nucleotides). In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell.

In some embodiments, the cells described herein are gene-edited to disrupt one or more of the genes encoding an MHC-I or MHC-II human leukocyte antigen, a component of a MHC-I or MHC-II complex, or a transcriptional regulator of a MHC-I or MHC-II complex. In some embodiments, the cells described herein are gene-edited to disrupt one or more of the genes encoding an MHC-I or MHC-II human leukocyte antigen. In some embodiments, the cells described herein are gene-edited to disrupt one or more of the genes encoding one or more components of a MHC-I or MHC-II complex. In some embodiments, the cells described herein are gene-edited to disrupt one or more of the genes encoding one or more transcriptional regulator of a MHC-I or MHC-II complex.

In some embodiments, the cells described herein are gene-edited to disrupt one or more genes including but not limited to: B2M, CIITA, ADAM17, CISH, CD38, FLI1, TGFβR1, TGFβR2, ZEB1, REGNASE1, FAS, TIGIT, PD-1, NKG2A, and/or ADORA2A/ADORA2B. In some embodiments, the cells described herein are gene-edited to disrupt B2M, CIITA, CISH, FAS, FLI1, ZEB1, TGFβR1, and/or TGFβR2. In some embodiments, the cells described herein are gene-edited to disrupt B2M. In some embodiments, the cells described herein are gene-edited to disrupt CIITA. In some embodiments, the cells described herein are gene-edited to disrupt ADAM17. In some embodiments, the cells described herein are gene-edited to disrupt CISH. In some embodiments, the cells described herein are gene-edited to disrupt REGNASE1. In some embodiments, the cells described herein are gene-edited to disrupt FAS. In some embodiments, the cells described herein are gene-edited to disrupt TIGIT. In some embodiments, the cells described herein are gene-edited to disrupt PD-1. In some embodiments, the cells described herein are gene-edited to disrupt NKG2A. In some embodiments, the cells described herein are gene-edited to disrupt FLI1. In some embodiments, the cells described herein are gene-edited to disrupt CD38. In some embodiments, the cells described herein are gene-edited to disrupt TGFβR1. In some embodiments, the cells described herein are gene-edited to disrupt TGFβR2. In some embodiments, the cells described herein are gene edited to disrupt ZEB1. In some embodiments, the cells described herein are gene edited to disrupt ADORA2A/ADORA2B.

In some embodiments, the cells described herein are gene-edited to insert a polynucleotide encoding, without limitation, one or more of the following: a tolerogenic factor, IL15, IL15Rα, HLA-E, SERPINB9, a CAR (e.g., against GPC3, GPR87, NKG2D, CD19, CD20, CD30, CD33, A33, BCMA, and/or combination thereof), CD16, and CD64. In some embodiments, the cells described herein are gene-edited to insert a polynucleotide encoding a fusion protein of IL15 and IL15Rα. In some embodiments, the cells described herein are gene-edited to insert a polynucleotide encoding a tolerogenic factor, such as HLA-E (e.g., wherein the HLA-E is a trimer comprising a B2M signal peptide fused to an HLA-G presentation peptide fused to the B2M membrane protein fused to the HLA-E protein without a signal peptide). In some embodiments, the cells described herein are gene-edited to insert a polynucleotide encoding SERPINB9. In some embodiments, the cells described herein are gene-edited to insert a SERPINB9-P2A-IL15/IL15Rα construct. In some embodiments, the cells described herein are gene-edited to insert a CAR-P2A-HLA-E construct, wherein the CAR is an anti-GPC3 CAR or an anti-GPR87 CAR. In some embodiments, the cells described herein are gene-edited to insert a polynucleotide encoding CD16 (e.g., a high affinity non-cleavable CD16). In some embodiments, the cells described herein are gene-edited to insert a polynucleotide encoding CD64. In some embodiments, the cells described herein are gene-edited to insert a polynucleotide encoding an NKG2D CAR.

In some embodiments, the cells described herein are gene-edited to insert a polynucleotide encoding, without limitation, one or more of the following: IL15/IL15Rα fusion, SERPINB9, CD16 (e.g., a high affinity non-cleavable CD16), CD64, and an NKG2D CAR. In some embodiments, the polynucleotide encoding SERPINB9-P2A-IL15/IL15Rα is inserted in a first location in the B2M gene locus (e.g., in exon 1 of the B2M gene locus), thereby disrupting expression of B2M. In some embodiments, the polynucleotide encoding an NKG2D CAR into a second location in the B2M gene locus. In some embodiments, the polynucleotide encoding CD16 is inserted into the CISH, FAS, FLI1, TGFβR1, or TGFβR2 gene, thereby disrupting said gene. In some embodiments, the polynucleotide encoding CD64 is inserted into the CISH, FAS, FLI1, TGFβR1, or TGFβR2 gene, thereby disrupting said gene. The disrupted expression can comprise reduced or eliminated expression of B2M, CISH, FAS, FLI1, TGFβR1, and/or TGFβR2

In some embodiments, the cells described herein are gene-edited to insert a polynucleotide encoding one or more of a CAR, HLA-E trimer, or a combination thereof into the CIITA gene locus, thereby disrupting expression of CIITA (e.g., the cell has reduced or eliminated expression of CIITA). In some embodiments, the cells described herein are gene-edited to insert any of the polynucleotides encoding the CAR and/or HLA-E in the disrupted CIITA gene locus (e.g., in exon 2 of the CIITA gene locus).

In some embodiments, the cells described herein are gene-edited to insert a polynucleotide encoding one or more chimeric antigen receptors (CARs). In some embodiments, and without limitation, the CAR is an anti-GPC3 CAR, and anti-GPR87 CAR, an NKG2D CAR, an anti-BCMA CAR, an anti-CD30 CAR, an anti-CD19 CAR, an anti-CD20 CAR, an anti-CD33 CAR, an anti-A33 CAR, an anti-CD70 CAR, an anti-NKp30 CAR, an anti-CD73 CAR, an anti-SLC7A11 (xCT) CAR, or a combination thereof (e.g., an anti-CD19-CD20-BCMA CAR). In some embodiments, the CAR is an anti-GPC3 CAR. In some embodiments, the CAR is an anti-GPR87 CAR. In some embodiments, the CAR is an anti-CD19-CD20-BCMA CAR. In some embodiments, the CAR is an anti-CD33 CAR. In some embodiments, the CAR is an anti-A33 CAR. In some embodiments, the CAR is an anti-NKG2D CAR. In some embodiments, the CAR is an anti-CD30 CAR. In some embodiments, the CAR is an anti-CD70 CAR. In some embodiments, the CAR is an anti-NKp30 CAR. In some embodiments, the CAR is an anti-CD73 CAR. In some embodiments, the CAR is an anti-SLC7A11 (xCT) CAR. In some embodiments, the cells described herein are gene-edited to insert a polynucleotide encoding a CAR, wherein the cell has a disrupted expression of CIITA (e.g., the cell is gene-edited to disrupt CIITA leading to, e.g., elimination of CIITA expression). In some embodiments, the CAR is inserted in the disrupted CIITA gene. In some embodiments, the CAR is inserted in exon 2 of the CIITA gene locus. In some embodiments, the cells described herein are gene-edited to insert a polynucleotide encoding a CAR, wherein the cell has a disrupted expression of B2M (e.g., the cell is gene-edited to disrupt B2M leading to, e.g., elimination of B2M expression). In some embodiments, the CAR is inserted in the disrupted B2M gene locus (e.g., in exon 1 of the B2M gene locus).

In some embodiments, the present disclosure provides a method of generating genome-engineered stem cells (e.g., iPSCs), wherein the stem cells comprise at least one targeted genomic modification at one or more selected sites in genome, the method comprising genetically engineering a cell type as described herein by introducing into said cells one or more constructs to allow targeted modification at a selected site; introducing into said cells one or more double strand breaks at the selected sites using one or more endonucleases capable of selected site recognition; and culturing the edited cells to allow endogenous DNA repair to generate targeted insertions or deletions at the selected sites; thereby obtaining genome-modified stem cells. The stem cells (e.g., iPSCs) generated by this method will comprise at least one functional targeted genomic modification, wherein the genome-modified cells, are then capable of being differentiated into progenitor cells or fully-differentiated cells (e.g., natural killer (NK) cells). In some embodiments, the differentiated cells (e.g., NK cells) maintain all of the gene-edits of the cells from which they were derived.

In some embodiments, a ribonucleoprotein particle (RNP) containing an RNA-guided nuclease (e.g., a Cas nuclease, such as a Cas9 nuclease) and a gRNA targeting the gene to be disrupted are delivered to any cell described herein (e.g., iPSC). An RNP is an RNA-guided nuclease (e.g., Cas9) pre-complexed/complexed with a gRNA. In other embodiments, the RNA-guided nuclease and gRNA are delivered separately to cells. In some embodiments, at least 50% of the engineered cells of a population of cells does not express a detectable level of the protein encoded by the disrupted gene. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of the disrupted gene product.

In some embodiments, at least 50% of the engineered cells of a population of cells expresses a detectable level of the protein encoded by the inserted polynucleotide. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of the protein encoded by the inserted polynucleotide.

### MHC I and MHC II Edits

Major histocompatibility complex I and II (MHC-I and MHC-II respectively) are cell surface proteins which perform an essential role in the adaptive immune system. The genes that encode the major histocompatibility complex (MHC) are located on human Chr. 6p21. The resultant proteins coded by the MHC genes are a series of surface proteins that are essential in donor compatibility during cellular transplantation. MHC genes are divided into MHC class I (MHC-I) and MHC class II (MHC-II). MHC-I genes (HLA-A, HLA-B, and HLA-C) are expressed in almost all tissue cell types, presenting “non-self” antigen-processed peptides to CD8+ T cells, thereby promoting their activation to cytolytic CD8+ T cells. Transplanted or engrafted cells expressing “non-self” MHC-I molecules will cause a robust cellular immune response directed at these cells and ultimately resulting in their demise by activated cytolytic CD8+ T cells. MHC-I proteins are intimately associated with beta-2-microglobulin (B2M) in the endoplasmic reticulum, which is essential for forming functional MHC-I molecules on the cell surface. In addition, there are three non-classical MHC-II molecules (HLA-E, HLA-F, and HLA-G), which have immune regulatory functions. MHC-II biomolecule include HLA-DP, HLA-DM, HLA-DOA, HLA-DOB, HLA-DQ, and HLA-DR. Due to their primary function in the immune response, MHC-I and MHC-II biomolecules contribute to immune rejection following cellular engraftment of non-host cells, e.g., cellular engraftment for purposes of regenerative medicine.

In some embodiments, a cell comprises a genomic modification of one or more MHC-I or MHC-II genes. In some embodiments, a cell comprises a genomic modification of one or more polynucleotide sequences that regulates the expression of MHC-I and/or MHC-II. In some embodiments, a genetic modification of the disclosure is performed using any gene editing method including but not limited to those methods described herein.

In some embodiments, any of the cells described herein have MHC-I and/or MHC-II genetic modifications. In some embodiments, MHC-I is disrupted. In some embodiments, MHC-II is disrupted. In some embodiments, both MHC-I and MHC-II are disrupted. In some embodiments, a MHC-I encoding gene is inserted. In some embodiments, a MHC-II encoding gene is inserted. In some embodiments, any genetically modified cell described herein comprises the introduction of at least one genetic modification within or near at least one gene that decreases the expression of one or more MHC-I and MHC-II human leukocyte antigens relative to an unmodified cell; at least one genetic modification that increases the expression of at least one polynucleotide that encodes a tolerogenic factor relative to an unmodified cell. In some embodiments, genetically modified cells comprise the introduction of at least one genetic modification within or near at least one gene that decreases the expression of one or more MHC-I and MHC-II human leukocyte antigens relative to an unmodified cell; at least one genetic modification that increases the expression of at least one polynucleotide that encodes a tolerogenic factor relative to an unmodified cell. In other embodiments, genetically modified cells comprise at least one deletion or insertion-deletion mutation within or near at least one gene that alters the expression of one or more MHC-I and MHC-II human leukocyte antigens relative to an unmodified cell; and at least one insertion of a polynucleotide that encodes at least one tolerogenic factor at a site that partially overlaps, completely overlaps, or is contained within, the site of a deletion of a gene that alters the expression of one or more MHC-I and MHC-II HLAS.

In some embodiments, decreasing the expression of one or more MHC-I and MHC-II human leukocyte antigens relative to an unmodified cell is accomplished by targeting, e.g., for genetic deletion and/or insertion of at least one base pair, in a MHC-I and/or MHC-II gene directly. In some embodiments, decreasing the expression of one or more MHC-I and MHC-II human leukocyte antigens relative to an unmodified cell is accomplished by targeting, e.g., for genetic deletion, a CIITA gene. In some embodiments, decreasing the expression of one or more MHC-I and MHC-II human leukocyte antigens relative to an unmodified cell is accomplished by targeting, e.g., for genetic deletion, at least one transcriptional regulator of MHC-I or MHC-II. In some embodiments, a transcriptional regulator of MHC-I or MHC-II is a NLRC5, or CIITA gene. In some embodiments, a transcriptional regulator of MHC-I or MHC-II is a RFX5, RFXAP, RFXANK, NFY-A, NFY-B, NFY-C, IRF-1, and/or TAP1 gene.

In some embodiments, the genome of a cell has been modified to delete the entirety or a portion of a HLA-A, HLA-B, and/or HLA-C gene. In some embodiments, the genome of a cell has been modified to delete the entirety or a portion of a promoter region of a HLA-A, HLA-B, and/or HLA-C gene. In some embodiments, the genome of a cell has been modified to delete the entirety or a portion of a gene that encodes a transcriptional regulator of MHC-I or MHC-II. In some embodiments, the genome of a cell has been modified to delete the entirety or a portion of a promoter region of a gene that encodes a transcriptional regulator of MHC-I or MHC-II.

MHC-I cell surface molecules are composed of MHC-encoded heavy chains (HLA-A, HLA-B, or HLA-C) and the invariant subunit beta-2-microglobulin (B2M). Thus, a reduction in the concentration of B2M within a cell allows for an effective method of reducing the cell surface expression of MHC-I cell surface molecules. In some embodiments, tolerogenic factors can be inserted or reinserted into genetically modified cells to create immune-privileged iPSCs or NK cells. In some embodiments, the iPSC or NK cells disclosed herein have been further modified to express one or more tolerogenic factors. Exemplary tolerogenic factors include, without limitation, one or more of HLA-C, HLA-E, HLA-F, HLA-G, PD-L1, CTLA-4-Ig, CD47, CI-inhibitor, and IL-35. In some embodiments, the genetic modification, e.g., insertion, of at least one polynucleotide encoding at least one tolerogenic factor enables a gene-edited iPSCs or NK cell to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, an insertion of a polynucleotide that encodes HLA-E, HLA-G, CTLA-4, CD47, and/or PD-L1 enables an iPSC or NK cell to inhibit or evade immune rejection after transplantation or engraftment into a host subject.

The polynucleotide encoding the tolerogenic factor generally comprises left and right homology arms that flank the sequence encoding the tolerogenic factor. The homology arms have substantial sequence homology to genomic DNA at or near the targeted insertion site. For example, the left homology arm can be a nucleotide sequence homologous with a region located to the left or upstream of the target site or cut site and the right homology arm can be a nucleotide sequence homologous with a region located to the right or downstream of the target site or cut site. The proximal end of each homology arm can be homologous to genomic DNA sequence abutting the cut site. Alternatively, the proximal end of each homology arm can be homologous to genomic DNA sequence located up to about 10, 20, 30, 40, 50, 60, or 70 nucleobases away from the cut site. As such, the polynucleotide encoding the tolerogenic factor can be inserted into the targeted gene locus within about 10, 20, 30, 40, 50, 60, or 70 base pairs of the cut site, and additional genomic DNA bordering the cut site (and having no homology to a homology arm) can be deleted. The homology arms can range in length from about 50 nucleotides to several of thousands of nucleotides. In some embodiments, the homology arms can range in length from about 500 nucleotides to about 1000 nucleotides. In some embodiments, the homology arms are 800 bp. In some embodiments, the substantial sequence homology between the homology arms and the genomic DNA is at least about 80%, at least about 85%, at least about 90%, at least about 95%, or at least about 99%.

In some embodiments, the at least one polynucleotide encoding at least one tolerogenic factor is operably linked to an exogenous promoter. In some embodiments, the exogenous promoter can be a constitutive, inducible, temporal-, tissue-, or cell type-specific promoter. In some embodiments, the exogenous promoter is a CAGGS, CMV, EF1α, PGK, CAG, or UBC promoter.

In some embodiments, the at least one polynucleotide encoding at least one tolerogenic factor is inserted into a safe harbor locus, e.g., the AAVS1 locus. In some embodiments, a safe harbor locus for inserting any gene described herein is selected from, but not limited to AAVS1 (PPP1 R12C), ALB, Angpt13, ApoC3, ASGR2, CCR5, FIX (F9), G6PC, Gys2, HGD, Lp (a), Pcsk9, Serpina1, TF, and TTR.

In some embodiments, the at least one polynucleotide encoding at least one tolerogenic factor is inserted into a site or region of genomic DNA that partially overlaps, completely overlaps, or is contained within (i.e., is within or near) a MHC-I gene, MHC-II gene, or a transcriptional regulator of MHC-I or MHC-II.

In some embodiments, the genome of a cell has been modified to decrease the expression of the NLR family, CARD domain containing 5 (NLRC5). NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, cells having no MHC-II expression and moderate expression of MHC-I are genetically modified to have no surface expression of MHC-I or MHC-II. In another embodiment, cells with no surface expression of MHC-I/II are further edited to have expression of programmed death ligand-1 (PD-L1), e.g., insertion of a polynucleotide encoding PD-L1. In yet another embodiment, cells with no surface expression of MHC-I/II are further edited to have expression of PD-L1, e.g., insertion of a polynucleotide encoding PD-L1.

In some embodiments, the cells further comprise increased or decreased expression, e.g., by a genetic modification, of one or more additional genes that are not necessarily implicated in either immune evasion or cell survival post-engraftment. In some embodiments, the cells further comprise increased expression of one or more safety switch proteins relative to an unmodified cell. In some embodiments, the cells comprise increased expression of one or more additional genes that encode a safety switch protein. In some embodiments, a safety switch is also a suicide gene. In some embodiments, a safety switch is herpes simplex virus-1 thymidine kinase (HSV-tk) or inducible caspase-9. In some embodiments, a polynucleotide that encodes at least one safety switch is inserted into a genome, e.g., into a safe harbor locus. In some other embodiments, the one or more additional genes that are genetically modified encode one or more of safety switch proteins; targeting modalities; receptors; signaling molecules; transcription factors; pharmaceutically active proteins or peptides; drug target candidates; and proteins promoting engraftment, trafficking, homing, viability, self-renewal, persistence, and/or survival thereof integrated with the construct.

### B2M Gene Edits

In some embodiments, the genome of a cell described herein is modified to disrupt beta-2-microglobulin (B2M or β2M) gene. B2M is a non-polymorphic gene that encodes a common protein subunit required for surface expression of all polymorphic MHC class I heavy chains. HLA-I proteins are intimately associated with B2M in the endoplasmic reticulum, which is essential for forming functional, cell-surface expressed HLA-I molecules. Disrupting its expression by gene editing will prevent host versus therapeutic cell responses leading to increased therapeutic cell persistence. In some embodiments, expression of the endogenous B2M gene is eliminated to prevent a host-versus-graft response. In some embodiments, the disrupted B2M can prevent allo-immune response due to MHC-I.

In some embodiments, any of the gene-editing methods described herein are used to disrupt the B2M gene. In some embodiments, any engineered cell described herein comprises a disrupted B2M gene. In some embodiments, an iPSC described herein comprises a disrupted B2M gene. In some embodiments, an NK cell described herein comprises a disrupted B2M gene.

In some embodiments, a ribonucleoprotein particle (RNP) containing an RNA-guided nuclease (e.g., a Cas nuclease, such as a Cas9 nuclease) and a gRNA targeting the B2M gene (or any other gene of interest) are delivered to any cell described herein (e.g., iPSC). A ribonucleoprotein particle (RNP) is a RNA-guided nuclease (e.g., Cas9) pre-complexed/complexed with a gRNA. In other embodiments, the RNA-guided nuclease and gRNA are delivered separately to cells. In some embodiments, the gRNA targets a site in the B2M gene. Non-limiting examples of modified and unmodified B2M gRNA sequences that may be used as provided herein to create a genomic disruption in the B2M gene include sequences corresponding to a sequence of any one of SEQ ID NOs: 34, 78 and 79. In some embodiments, a gRNA is used to target the B2M site for gene-editing. In some embodiments, the gRNA comprises a sequence of SEQ ID NO: 180, 187, or 188. See also International Application No. PCT/US2018/032334, filed May 11, 2018, incorporated herein by reference. Other gRNA sequences may be designed using the B2M (B2M) gene sequence located on Chromosome 15 (GRCh38 coordinates: Chromosome 15:44,711,477-44,718,877; Ensembl: ENSG00000166710). In some embodiments, any B2M RNP described herein is used in combination with a donor plasmid containing B2M homology arms for insertion of any polynucleotide described herein.

In some embodiments, a gRNA/CRISPR nuclease complex targets and cleaves a target site in exon 1 of the B2M gene locus. In some embodiments, the B2M gRNA targets a sequence comprising SEQ ID NOs: 34, 78, or 79. Repair of a double-stranded break by NHEJ can result in a deletion of at least on nucleotide and/or an insertion of at least one nucleotide, thereby disrupting or eliminating expression of B2M. In some embodiments, the B2M gene locus is targeted by at least two CRISPR systems each comprising a different gRNA, such that cleavage at two sites in the B2M gene locus leads to a deletion of the sequence between the two cuts, thereby eliminating expression of B2M.

In some embodiments, the homology arms are used with B2M guides (e.g., gRNA comprising the nucleotide sequence of SEQ ID NO: 180). In some embodiments, the homology arms are designed to be used with any B2M guide that would eliminate the start site of the B2M gene (e.g., within exon 1 of the B2M gene). In some embodiments, the B2M homology arms comprise or consist of a polynucleotide of the sequence of SEQ ID NOs: 36 and 54, or polynucleotides having at least 85%, 90%, 95%, or 99% sequence identity to SEQ ID NOs: 36 or 54. In some embodiments, the left B2M homology arm can comprise or consist of SEQ ID NO: 36, or a polynucleotide sequence having at least 85%, 90%, 95%, or 99% sequence identity to SEQ ID NO: 36. In some embodiments, the right B2M homology arm can comprise or consist of SEQ ID NO: 54, or a polynucleotide sequence having at least 85%, 90%, 95%, or 99% sequence identity to SEQ ID NO:54. In some embodiments, gRNAs targeting the B2M genomic region create indels in the B2M gene disrupting expression of the mRNA or protein.

In some embodiments, a first polynucleotide is inserted into exon 1 of the B2M gene and a second polynucleotide is inserted into a location other than exon 1 of the B2M gene. For example, a polynucleotide encoding SERPINB9-P2A-IL15/IL15Rα is inserted into exon 1 of the B2M gene using the gRNAs and homology arms detailed above, and a polynucleotide encoding an NKG2D CAR is inserted into a location other than exon 1 of the B2M gene using gRNAs and homology arms other than those described above.

In some embodiments, at least 50% of the engineered cells of a population of cells do not express a detectable level of B2M surface protein. For example, at least 55%, at least 60%, at least 70%, at least 75%, at least 80%, at least 85%, at least 90%, or at least 95% of the engineered cells of a population may not express a detectable level of B2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of B2M surface protein.

In some embodiments, less than 50% of the engineered cells of a population of cells express a detectable level of B2M surface protein. In some embodiments, less than 30% of the engineered cells of a population of cells express a detectable level of B2M surface protein. For example, less than 50%, less than 30%, less than 25%, less than 20%, less than 15%, less than 10%, or less than 5% of the engineered cells of a population of cells express a detectable level of B2M surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of B2M surface protein.

### CIITA Gene Edits

In some embodiments, the genome of a cell described herein is modified to disrupt Class II transactivator (CIITA). CIITA is a member of the LR or nucleotide binding domain (NBD) leucine-rich repeat (LRR) family of proteins and regulates the transcription of MHC-II by associating with the MHC enhanceosome. The expression of CIITA is induced in B cells and dendritic cells as a function of developmental stage and is inducible by IFN-γ in most cell types. In some embodiments, the disrupted CIITA gene locus can prevent allo-immune response due to MHC-II.

In some embodiments, a gene-editing method described herein is used to disrupt the CIITA gene. In some embodiments, any engineered cell described herein comprises a disrupted CIITA gene. In some embodiments, an iPSC described herein comprises a disrupted CIITA gene. In some embodiments, an NK cell described herein comprises a disrupted CIITA gene.

In some embodiments, a ribonucleoprotein particle (RNP) containing an RNA-guided nuclease (e.g., a Cas nuclease, such as a Cas9 nuclease) and a gRNA targeting the CIITA gene (or any other gene of interest) are delivered to any cell described herein (e.g., iPSC). A ribonucleoprotein particle (RNP) is an RNA-guided nuclease (e.g., Cas9) pre-complexed/complexed with a gRNA. In other embodiments, the RNA-guided nuclease and gRNA are delivered separately to cells. Non-limiting examples of modified and unmodified CIITA gRNA sequences that may be used as provided herein to create a genomic disruption in the CIITA gene are listed in Table 15 (e.g., corresponding to target sequence of any of SEQ ID NOs: 13-17 and/or comprising the RNA sequence of any of SEQ ID NOs: 175-179). In some embodiments, the gRNA targets a site within the CIITA gene. In some embodiments, the CIITA gRNA targets a sequence comprising any of SEQ ID NOs: 13-17. In some embodiments, the gRNA comprises an RNA sequence corresponding to the sequence of SEQ ID NO: 13. In some embodiments, the gRNA comprises the sequence of SEQ ID NO: 175. In some embodiments, any CIITA RNP described herein is used in combination with a donor plasmid containing CIITA homology arms for insertion of any polynucleotide described herein.

In some embodiments, gRNAs targeting the CIITA genomic region create indels in the CIITA gene disrupting expression of the mRNA and/or protein. In some embodiments, a gRNA/CRISPR nuclease complex targets and cleaves a target site in the CIITA gene locus. Repair of a double-stranded break by NHEJ can result in a deletion of at least on nucleotide and/or an insertion of at least one nucleotide, thereby disrupting or eliminating expression of CIITA. In some embodiments, the CIITA gene locus is targeted by at least two CRISPR systems each comprising a different gRNA, such that cleavage at two sites in the CIITA gene locus leads to a deletion of the sequence between the two cuts, thereby eliminating expression of CIITA.

In some embodiments, the homology arms are used with CIITA guides (e.g., gRNAs comprising a sequence of any one of SEQ ID NOs: 175-179). In some embodiments, the homology arms are designed to be used with any CIITA guide that would eliminate the start site of the CIITA gene. In some embodiments, the CIITA homology arms comprise or consist of polynucleotides of SEQ ID NOs: 22 and 32, or polynucleotide sequences having at least 85%, 90%, 95%, or 99% sequence identity to SEQ ID NOs: 22 or 32. In some embodiments, the left CIITA homology arm can comprise or consist of SEQ ID NO: 22, or a polynucleotide sequence having at least 85%, 90%, 95%, or 99% sequence identity to SEQ ID NO: 22. In some embodiments, the right CIITA homology arm can comprise or consist of SEQ ID NO: 32, or a polynucleotide sequence having at least 85%, 90%, 95%, or 99% sequence identity to SEQ ID NO: 32.

In some embodiments, at least 50% of the engineered cells of a population of cells do not express a detectable level of CIITA protein. For example, at least 55%, at least 60%, at least 70%, at least 75%, at least 80%, at least 85%, at least 90%, or at least 95% of the engineered cells of a population may not express a detectable level of CIITA surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of CIITA protein.

In some embodiments, less than 50% of the engineered cells of a population of cells express a detectable level of CIITA protein. In some embodiments, less than 30% of the engineered cells of a population of cells express a detectable level of CIITA protein. For example, less than 50%, less than 30%, less than 25%, less than 20%, less than 15%, less than 10%, or less than 5% of the engineered cells of a population of cells express a detectable level of CIITA protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CIITA protein. In some embodiments, any polynucleotide described herein is inserted into the CIITA gene locus such that 86 base pairs (bp) of the CIITA exon 2 are removed after homology directed repair.

### HLA-E Gene Edits

In some embodiments, the genome of a cell described herein comprises an inserted HLA-E gene. HLA-E is a heterodimer class I molecule. HLA-E primarily functions as a ligand for the NK cell inhibitory receptor KLRD1-KLRC1. HLA-E enables NK cells to monitor other MHC class I molecule expression and to tolerate self-expression. In some embodiments, the insertion of the HLA-E can protect the iNK from PB-NK “missing self” response. In some embodiments, expression of HLA-E is increased in cells. In some embodiments, an iPSC comprises a knock-in HLA-E gene. In some embodiments, an NK cell comprises a knock-in HLA-E gene.

Non-limiting examples of modified and unmodified HLA-E cDNA sequences that may be used as provided herein to create a genomic knock-in of the HLA-E gene include SEQ ID NOs: 51 and 75 (e.g., HLA-E trimer). In some embodiments, the HLA-E trimer polynucleotide has at least 90%, 91%, 92%, 93%, 94%, 95%, 96%, 97%, 98%, or 99% identity to SEQ ID NO: 75. In some embodiments, the HLA trimer has the amino acid sequence of SEQ ID NO: 142.

In some embodiments, at least 50% of the engineered cells of a population of cells express a detectable level of HLA-E surface protein. For example, at least 55%, at least 60%, at least 70%, at least 75%, at least 80%, at least 85%, at least 90%, or at least 95% of the engineered cells of a population express a detectable level of HLA-E surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of HLA-E surface protein.

In some embodiments, less than 50% of the engineered cells of a population of cells do not express a detectable level of HLA-E surface protein. In some embodiments, less than 30% of the engineered cells of a population of cells do not express a detectable level of HLA-E surface protein. For example, less than 50%, less than 30%, less than 25%, less than 20%, less than 15%, less than 10%, or less than 5% of the engineered cells of a population of cells do not express a detectable level of HLA-E surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells do not express a detectable level of HLA-E surface protein.

In some embodiments, any of the HLA-E polynucleotides described herein are inserted into any safe-harbor locus described herein. In some embodiments, any of the HLA-E polynucleotides described herein are inserted into any B2M gene locus described herein. In some embodiments, the HLA-E polynucleotide is an HLA-E trimer composed of a B2M signal peptide fused to an HLA-G presentation peptide fused to the B2M membrane protein fused to the HLA-E protein without its signal peptide. In some embodiments, the HLA-E trimer comprises or consists of SEQ ID NO: 75. In some embodiments, the HLA-E polynucleotide has at least 90%, 91%, 92%, 93%, 94%, 95%, 96%, 97%, 98%, or 99% identity to SEQ ID NO: 75. In some embodiments, the trimer design is that described in Gornalusse et al. (2017) Nat. Biotechnol. 35 (8): 765-772, which is incorporated by reference herein in its entirety.

### IL15 and IL15Rα Gene Edits

In some embodiments, the genome of a cell described herein comprises an insertion of a polynucleotide encoding interleukin-15 (IL15) gene. IL15 is a cytokine that functions in regulating NK cell proliferation and activation. In some embodiments, the genome of any cell described herein comprises an insertion of a polynucleotide encoding interleukin-15 receptor alpha (IL15Rα, also referred to herein as IR15α), the receptor that binds IL15. In some embodiments, the genome of any cell described herein comprises an insertion of a polynucleotide encoding a fusion protein of IL15 and IL15Rα. In some embodiments, the insertion of the IL15, IL15Rα, and/or fusion protein of IL15 and IL15Rα can lead to increased iNK persistence of the engineered cell. In some embodiments, a cell has insertion of a polynucleotide comprising the sequence of SEQ ID NO: 41. In some embodiments, a cell has insertion of a polynucleotide comprising the sequence of SEQ ID NO: 43. In some embodiments, a cell has insertion of a polynucleotide encoding both IL15 and IL15Rα proteins. In some embodiments, IL15 and IL15Rα are co-expressed. In some embodiments, the IL15/IL15Rα fusion sequence is used as described in Hurton et al. (2016) Proc Natl Acad Sci U SA.; 113 (48): E7788-E7797. doi: 10.1073/pnas.1610544113, which is incorporated herein in its entirety. In some embodiments, the IL15/IL15Rα polynucleotide is SEQ ID NO: 76. In some embodiments, the IL15/IL15Rα polynucleotide has at least 90%, 91%, 92%, 93%, 94%, 95%, 96%, 97%, 98%, or 99% identity to SEQ ID NO: 76. In some embodiments, the IL15/IL15Rα fusion has the amino acid sequence of SEQ ID NO: 143.

A self-cleaving peptide can be used to co-express IL15 and IL15Rα. In some embodiments, the self-cleaving peptide is selected from, but not limited to, P2A, E2A, F2A, and T2A. In some embodiments, the self-cleaving peptide is P2A. In some embodiments, a cell has insertion of a polynucleotide encoding IL15, IL15Rα, and P2A (IL15/IL15Rα-P2A). In some embodiments, an iPSC comprises a knock-in of the IL15/IL15Rα polynucleotide. In some embodiments, an NK cell comprises a knock-in of the IL15/IL15Rα polynucleotide.

In some embodiments, at least 50% of the engineered cells of a population of cells express a detectable level of any IL15 and/or IL15Rα polynucleotide described herein. For example, at least 55%, at least 60%, at least 70%, at least 75%, at least 80%, at least 85%, at least 90%, or at least 95% of the engineered cells of a population express a detectable level of IL15 and/or IL15Rα. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population expresses a detectable level of IL15 and/or IL15Rα.

In some embodiments, at least 50% of the engineered cells of a population of cells express a detectable level of IL15/IL15Rα. For example, at least 55%, at least 60%, at least 70%, at least 75%, at least 80%, at least 85%, at least 90%, or at least 95% of the engineered cells of a population express a detectable level of IL15/IL15Rα.

In some embodiments, a IL15/IL15Rα polynucleotides described herein are inserted into any safe-harbor locus described herein. In some embodiments, any of the IL15/IL15Rα polynucleotides described herein are inserted into any B2M gene locus described herein.

### SERPINB9 Gene Edits

In some embodiments, the genome of a cell described herein comprises an inserted polynucleotide encoding SERPINB9 (NCBI Gene ID: 5272). SERPINB9 is a member of a large family of apoptosis inhibitors that mainly function by targeting intermediate proteases (e.g., covalently bind a protease in 1:1 complex, thereby inhibiting the protease). As such, expression of SERPINB9 may increase survival of the engineered cells. For example, iNK cells engineered to express SERPINB9 can survive NK cell attack by inhibiting activity of the released granzymes. In some embodiments, expression of SERPINB9 is increased in cells. In some embodiments, an iPSC comprises a knock-in of the SERPINB9 gene. In some embodiments, an NK cell comprises a knock-in SERPINB9 gene.

An example of a SERPINB9 cDNA sequence that may be used as provided herein to create a genomic knock-in of the SERPINB9 gene is SEQ ID NO: 129. In some embodiments, the SERPINB9 polynucleotide has at least 90%, 91%, 92%, 93%, 94%, 95%, 96%, 97%, 98%, or 99% identity to SEQ ID NO: 129. In some embodiments, the SERPINB9 protein has the amino acid sequence of SEQ ID NO: 144.

In some embodiments, at least 50% of the engineered cells of a population of cells express a detectable level of SERPINB9 protein. For example, at least 55%, at least 60%, at least 70%, at least 75%, at least 80%, at least 85%, at least 90%, or at least 95% of the engineered cells of a population express a detectable level of SERPINB9 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of SERPINB9 protein.

In some embodiments, a SERPINB9 polynucleotides described herein is inserted into any safe-harbor locus described herein. In some embodiments, a SERPINB9 polynucleotides described herein is inserted into any B2M gene locus described herein.

### ADAM17 Gene Edits

In some embodiments, the genome of a cell described herein is modified to disrupt the ADAM17 gene (NCBI Gene ID: 6868). ADAM Metallopeptidase Domain 17 (ADAM17) that cleaves TNF-α precursor. ADAM17 is responsible for proteolytic cleavage of several surface proteins. In some embodiments, the disrupted ADAM17 can increase ADCC killing by preventing CD16 cleavage.

In some embodiments, a gene-editing methods described herein is used to disrupt the ADAM17 gene. In some embodiments, an iPSC comprises a disrupted ADAM17 gene. In some embodiments, an NK cell comprises a disrupted ADAM17 gene.

In some embodiments, a RNP containing an RNA-guided nuclease (e.g., a Cas nuclease, such as a Cas9 nuclease) and a gRNA targeting the ADAM17 gene (or any other gene of interest) are delivered to any cell described herein (e.g., iPSC). A RNP is RNA-guided nuclease (e.g., Cas9) pre-complexed/complexed with a gRNA. In some embodiments, the RNA-guided nuclease and gRNA are delivered separately to cells.

Non-limiting examples of modified and unmodified ADAM17 gRNA sequences that may be used as provided herein to create a genomic disruption in the ADAM17 gene include RNA sequences corresponding to sequences of any of SEQ ID NOs: 1-10. In some embodiments, the ADAM17 gRNA comprises the sequence of any one of SEQ ID NOs: 165-174. In some embodiments, the ADAM17 gRNA targets a sequence comprising any one of SEQ ID NOs: 1-10.

In some embodiments, gRNAs targeting the ADAM17 genomic region create indels in the ADAM17 gene disrupting expression of the mRNA or protein. In some embodiments, at least 50% of the engineered cells of a population of cells do not express a detectable level of ADAM17 protein. For example, at least 55%, at least 60%, at least 70%, at least 75%, at least 80%, at least 85%, at least 90%, or at least 95% of the engineered cells of a population may not express a detectable level of ADAM17 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of ADAM17 protein. In some embodiments, less than 50% of the engineered cells of a population of cells express a detectable level of ADAM17 protein. In some embodiments, less than 30% of the engineered cells of a population of cells express a detectable level of ADAM17 protein. For example, less than 50%, less than 30%, less than 25%, less than 20%, less than 15%, less than 10%, or less than 5% of the engineered cells of a population of cells express a detectable level of ADAM17 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of ADAM17 protein.

### CISH Gene Edits

In some embodiments, the genome of a cell described herein is modified to disrupt the CISH (also called CIS) gene encoding cytokine inducible SH2 containing protein (NCBI Gene ID: 1154). CISH is a transcriptional co-activator that controls expression of HLA class II genes. In some embodiments, the disrupted CISH can increase iNK sensitivity to cytokines, improve iNK persistence, and/or increase tumor killing. In some embodiments, an iPSC comprises a disrupted CISH gene. In some embodiments, an NK cell comprises a disrupted CISH gene.

In some embodiments, gRNAs targeting the CISH genomic region create indels in the CISH gene disrupting expression of the mRNA and/or protein. In some embodiments, the gRNA targets a site within the CISH gene. In some embodiments, the CISH gRNA targets a sequence comprising any one of SEQ ID NOs: 81-92. In some embodiments, a gRNA targeting the CISH gene comprises an RNA spacer sequence corresponding to a sequence comprising any one of SEQ ID NOs: 81-92. In some embodiments, a gRNA targeting the CISH gene comprises a spacer sequence of any one of SEQ ID NOs: 192-201.

In some embodiments, at least 50% of the engineered cells of a population of cells do not express a detectable level of CISH protein. For example, at least 55%, at least 60%, at least 70%, at least 75%, at least 80%, at least 85%, at least 90%, or at least 95% of the engineered cells of a population may not express a detectable level of CISH surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of CISH protein. In some embodiments, less than 50% of the engineered cells of a population of cells express a detectable level of CISH protein. In some embodiments, less than 30% of the engineered cells of a population of cells express a detectable level of CISH protein. For example, less than 50%, less than 30%, less than 25%, less than 20%, less than 15%, less than 10%, or less than 5% of the engineered cells of a population of cells express a detectable level of CISH surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CISH protein.

### REGNASE-1 Gene Edits

In some embodiments, the genome of a cell described herein is modified to disrupt the REGNASE-1 gene (also called Zinc Finger CCCH-Type Containing 12A; NCBI Gene ID: 80149). REGNASE-1 is an endoribonuclease involved in mRNA decay. In some embodiments, the disrupted REGNASE-1 can increase iNK persistence and/or increase tumor killing. In some embodiments, an iPSC comprises a disrupted REGNASE-1 gene. In some embodiments, an NK cell comprises a disrupted REGNASE-1 gene.

In some embodiments, gRNAs targeting the REGNASE-1 genomic region create indels in the REGNASE-1 gene disrupting expression of the mRNA or protein. In some embodiments, the gRNA targets a site within the REGNASE-1 gene. In some embodiments, the REGNASE-1 gRNA targets a sequence comprising any one of SEQ ID NOs: 93-101. In some embodiments, a gRNA targeting the REGNASE-1 gene comprises an RNA spacer sequence corresponding to a sequence comprising any one of SEQ ID NOs: 93-101. In some embodiments, a gRNA targeting the REGNASE-1 gene comprises a spacer sequence of any one of SEQ ID NOs: 202-210.

In some embodiments, at least 50% of the engineered cells of a population of cells do not express a detectable level of REGNASE-1 protein. For example, at least 55%, at least 60%, at least 70%, at least 75%, at least 80%, at least 85%, at least 90%, or at least 95% of the engineered cells of a population may not express a detectable level of REGNASE-1 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of REGNASE-1 protein. In some embodiments, less than 50% of the engineered cells of a population of cells express a detectable level of REGNASE-1 protein. In some embodiments, less than 30% of the engineered cells of a population of cells express a detectable level of REGNASE-1 protein. For example, less than 50%, less than 30%, less than 25%, less than 20%, less than 15%, less than 10%, or less than 5% of the engineered cells of a population of cells express a detectable level of REGNASE-1 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of REGNASE-1 protein.

### FAS Gene Edits

In some embodiments, the genome of a cell described herein is modified to disrupt the FAS gene (NCBI Gene ID: 355). FAS is a member of the TNF-receptor superfamily and contributes to the regulation of programmed cell death. In some embodiments, the disrupted FAS reduce activation-induced cell death (AICD), resist apoptosis, and/or increase tumor killing. In some embodiments, an iPSC comprises a disrupted FAS gene. In some embodiments, an NK cell comprises a disrupted FAS gene.

In some embodiments, gRNAs targeting the FAS genomic region create indels in the FAS gene disrupting expression of the mRNA or protein. In some embodiments, the gRNA targets a site within the FAS gene. In some embodiments, the FAS gRNA targets a sequence comprising any one of SEQ ID NOs: 35, 37, 38, 39, 53, 55, and 80. In some embodiments, a gRNA targeting the FAS gene comprises an RNA spacer sequence corresponding to a sequence comprising any one of SEQ ID NOs: 35, 37, 38, 39, 53, 55, and 80. In some embodiments, a gRNA targeting the FAS gene comprises a spacer sequence of any one of SEQ ID NOs: 181-186 and 189.

In some embodiments, at least 50% of the engineered cells of a population of cells do not express a detectable level of FAS protein. For example, at least 55%, at least 60%, at least 70%, at least 75%, at least 80%, at least 85%, at least 90%, or at least 95% of the engineered cells of a population may not express a detectable level of FAS surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of FAS protein. In some embodiments, less than 50% of the engineered cells of a population of cells express a detectable level of FAS protein. In some embodiments, less than 30% of the engineered cells of a population of cells express a detectable level of FAS protein. For example, less than 50%, less than 30%, less than 25%, less than 20%, less than 15%, less than 10%, or less than 5% of the engineered cells of a population of cells express a detectable level of FAS protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of FAS protein.

### CD38 Gene Edits

In some embodiments, the genome of a cell described herein is modified to disrupt the CD38 gene (NCBI Gene ID: 952). The CD38 gene encodes cyclic ADP ribose hydrolase. CD38 is often overexpressed in certain cancers and is a marker of cell activation. In some embodiments, a disrupted CD38 gene can increase daratumumab induced ADCC killing and/or increase iNK cell fitness. In some embodiments, an iPSC comprises a disrupted CD38 gene. In some embodiments, an NK cell comprises a disrupted CD38 gene.

In some embodiments, at least 50% of the engineered cells of a population of cells do not express a detectable level of CD38 protein. For example, at least 55%, at least 60%, at least 70%, at least 75%, at least 80%, at least 85%, at least 90%, or at least 95% of the engineered cells of a population may not express a detectable level of CD38 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of CD38 protein.

In some embodiments, less than 50% of the engineered cells of a population of cells express a detectable level of CD38 protein. In some embodiments, less than 30% of the engineered cells of a population of cells express a detectable level of CD38 protein. For example, less than 50%, less than 30%, less than 25%, less than 20%, less than 15%, less than 10%, or less than 5% of the engineered cells of a population of cells express a detectable level of CD38 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CD38 protein.

### FLI1 Gene Edits

In some embodiments, the genome of a cell described herein is modified to disrupt the FLI1 (Friend leukemia integration 1 transcription factor) gene (NCBI Gene ID: 2313). FLI1, also known as transcription factor ERGB, is a member of the ETS transcription factor family that controls expression of genes related to erythroblast response to erythropoietin and their subsequent proliferation and differentiation. In some embodiments, the disrupted FLI1 gene can improve NK92 cytotoxicity. In some embodiments, an iPSC comprises a disrupted FLI1 gene. In some embodiments, an NK cell comprises a disrupted FLI1 gene.

In some embodiments, at least 50% of the engineered cells of a population of cells do not express a detectable level of FLI1 protein. For example, at least 55%, at least 60%, at least 70%, at least 75%, at least 80%, at least 85%, at least 90%, or at least 95% of the engineered cells of a population may not express a detectable level of FLI1 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of FLI1 protein.

In some embodiments, less than 50% of the engineered cells of a population of cells express a detectable level of FLI1 protein. In some embodiments, less than 30% of the engineered cells of a population of cells express a detectable level of FLI1 protein. For example, less than 50%, less than 30%, less than 25%, less than 20%, less than 15%, less than 10%, or less than 5% of the engineered cells of a population of cells express a detectable level of FLI1 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of FLI1 protein.

### TGFβR1/TGFβR2 Gene Edits

In some embodiments, the genome of a cell described herein is modified to disrupt the TGFβR1 (transforming growth factor beta receptor 1, also referred to herein as TGFβR1) gene (NCBI Gene ID: 7046) and/or the TGFβR2 (transforming growth factor beta receptor 2, also referred to herein as TGFβR2) gene (NCBI Gene ID: 7048). TGF-β is a negative regulator of IFN-γ production and decreases the surface level of the activating receptors NKG2D and NKp30, reducing the cytotoxic ability of NK cells and impairing their antitumor function. In some embodiments, the disrupted TGFβR1 and/or TGFβR2 gene can increase persistence and cytotoxicity of CAR-T and NK cells. In some embodiments, an iPSC comprises a disrupted TGFβR1/TGFβR2 (e.g., disrupted TGFβR1 gene and/or disrupted TGFβR2 gene). In some embodiments, an NK cell comprises a disrupted TGFβR1/TGFβR2 gene.

In some embodiments, at least 50% of the engineered cells of a population of cells do not express a detectable level of TGFβR1/TGFβR2 protein. For example, at least 55%, at least 60%, at least 70%, at least 75%, at least 80%, at least 85%, at least 90%, or at least 95% of the engineered cells of a population may not express a detectable level of TGFβR1/TGFβR2 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of TGFβR1/TGFβ2 protein.

In some embodiments, less than 50% of the engineered cells of a population of cells express a detectable level of TGFβR1/TGFβR2 protein. In some embodiments, less than 30% of the engineered cells of a population of cells express a detectable level of TGFβR1/TGFβR2 protein. For example, less than 50%, less than 30%, less than 25%, less than 20%, less than 15%, less than 10%, or less than 5% of the engineered cells of a population of cells express a detectable level of TGFβR1/TGFβR2 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of TGFβR1/TGFβR2 protein.

In some embodiments, gRNAs targeting the TGFβR2 genomic region create indels in the TGFβR2 gene disrupting expression of the mRNA or protein. In some embodiments, the gRNA targets a site within the TGFβR2 gene. In some embodiments, the TGFβR2 gRNA targets a sequence comprising SEQ ID NO: 151. In some embodiments, a gRNA targeting the TGFβR2 gene comprises an RNA sequence corresponding to a sequence comprising SEQ ID NO: 151. In some embodiments, a gRNA targeting the TGFβR2 gene comprises a sequence of SEQ ID NO: 211.

### ZEB1 Gene Edits

In some embodiments, the genome of a cell described herein is modified to disrupt the ZEB1 (zinc-finger E homeobox-binding-1) gene (NCBI Gene ID: 6935). ZEB1 protein likely plays a role in transcriptional repression of interleukin 2. Mutations in ZEB1 gene have been associated with posterior polymorphous corneal dystrophy-3 and late-onset Fuchs endothelial corneal dystrophy. In some embodiments, the disrupted ZEB1 gene can increase persistence of the genetically engineered cell. In some embodiments, an iPSC comprises a disrupted ZEB1 gene. In some embodiments, an NK cell comprises a disrupted ZEB1 gene.

In some embodiments, at least 50% of the engineered cells of a population of cells do not express a detectable level of ZEB1 protein. For example, at least 55%, at least 60%, at least 70%, at least 75%, at least 80%, at least 85%, at least 90%, or at least 95% of the engineered cells of a population may not express a detectable level of ZEB1 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of ZEB1 protein.

In some embodiments, less than 50% of the engineered cells of a population of cells express a detectable level of ZEB1 protein. In some embodiments, less than 30% of the engineered cells of a population of cells express a detectable level of ZEB1 protein. For example, less than 50%, less than 30%, less than 25%, less than 20%, less than 15%, less than 10%, or less than 5% of the engineered cells of a population of cells express a detectable level of ZEB1 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of ZEB 1 protein.

### PD-1 Gene Edits

In some embodiments, the genome of a cell described herein is modified to disrupt the PD-1 (receptor of Programmed cell death protein 1 or PDCD1) gene (NCBI Gene ID: 5133). PD-1 is an immune-inhibitory receptor expressed in activated T cells; it is involved in the regulation of T-cell functions, including those of effector CD8+ T cells. In addition, this protein can also promote the differentiation of CD4+ T cells into T regulatory cells. PD-1 is expressed in many types of tumors including melanomas, and has demonstrated to play a role in anti-tumor immunity. Moreover, this protein has been shown to be involved in safeguarding against autoimmunity. In some embodiments, the disrupted PD-1 gene can increase activity and cytotoxicity of NK cells. In some embodiments, an iPSC comprises a disrupted PD-l gene. In some embodiments, an NK cell comprises a disrupted PD-l gene.

In some embodiments, at least 50% of the engineered cells of a population of cells do not express a detectable level of PD-1 protein. For example, at least 55%, at least 60%, at least 70%, at least 75%, at least 80%, at least 85%, at least 90%, or at least 95% of the engineered cells of a population may not express a detectable level of PD-1 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of PD-1 protein.

In some embodiments, less than 50% of the engineered cells of a population of cells express a detectable level of PD-1 protein. In some embodiments, less than 30% of the engineered cells of a population of cells express a detectable level of PD-1 protein. For example, less than 50%, less than 30%, less than 25%, less than 20%, less than 15%, less than 10%, or less than 5% of the engineered cells of a population of cells express a detectable level of PD-1 protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of PD-1 protein.

### TIGIT Gene Edits

In some embodiments, the genome of a cell described herein is modified to disrupt the TIGIT (T cell immunoreceptor with Ig and ITIM domains) gene (NCBI Gene ID: 201633). TIGIT encodes a member of the PVR (poliovirus receptor) family of immunoglobin proteins. The product of this gene is expressed on several classes of T cells including follicular B helper T cells (TFH). The TIGIT protein has been shown to bind PVR with high affinity; this binding is thought to assist interactions between TFH and dendritic cells to regulate T cell dependent B cell responses. In some embodiments, the disrupted TIGIT gene can increase activity and cytotoxicity of NK cells. In some embodiments, an iPSC comprises a disrupted TIGIT gene. In some embodiments, an NK cell comprises a disrupted TIGIT gene.

In some embodiments, at least 50% of the engineered cells of a population of cells do not express a detectable level of TIGIT protein. For example, at least 55%, at least 60%, at least 70%, at least 75%, at least 80%, at least 85%, at least 90%, or at least 95% of the engineered cells of a population may not express a detectable level of TIGIT protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of TIGIT protein.

In some embodiments, less than 50% of the engineered cells of a population of cells express a detectable level of TIGIT protein. In some embodiments, less than 30% of the engineered cells of a population of cells express a detectable level of TIGIT protein. For example, less than 50%, less than 30%, less than 25%, less than 20%, less than 15%, less than 10%, or less than 5% of the engineered cells of a population of cells express a detectable level of TIGIT protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of TIGIT protein.

### NKG2A Gene Edits

In some embodiments, the genome of a cell described herein is modified to disrupt the NKG2A (Natural Killer Group Protein 2) gene (NCBI Gene ID: 3821). This gene is also known as Killer Cell Lectin-Like Receptor Subfamily C, Member 1 (KLRC1) or CD159. The NKG2A gene is a member of a family of NK cell receptors. NKG2A can dimerize with CD94 to form an inhibitory receptor which recognize HLA-E and suppress NK cell function. In some embodiments, the disrupted NKG2A gene can overcome HLA-E inhibition from cancer cells. In some embodiments, an iPSC comprises a disrupted NKG2A gene. In some embodiments, an NK cell comprises a disrupted NKG2A gene.

In some embodiments, at least 50% of the engineered cells of a population of cells do not express a detectable level of NKG2A protein. For example, at least 55%, at least 60%, at least 70%, at least 75%, at least 80%, at least 85%, at least 90%, or at least 95% of the engineered cells of a population may not express a detectable level of NKG2A protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of NKG2A protein.

In some embodiments, less than 50% of the engineered cells of a population of cells express a detectable level of NKG2A protein. In some embodiments, less than 30% of the engineered cells of a population of cells express a detectable level of NKG2A protein. For example, less than 50%, less than 30%, less than 25%, less than 20%, less than 15%, less than 10%, or less than 5% of the engineered cells of a population of cells express a detectable level of NKG2A protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of NKG2A protein.

### ADORA2A/ADORA2B Gene Edits

In some embodiments, the genome of a cell described herein is modified to disrupt the ADORA2A (adenosine A2a receptor) gene (NCBI Gene ID: 135) and/or the ADORA2B (adenosine A2b receptor) gene (NCBI Gene ID: 136). Activation of the A2a/A2b receptors acts as a checkpoint that limits the maturation of natural killer (NK) cells. In some embodiments, the disrupted ADORA2A/ADORA2B gene can remove adenosine-based inhibition of NK cells. In some embodiments, an iPSC comprises a disrupted ADORA2A/ADORA2B gene. In some embodiments, an NK cell comprises a disrupted ADORA2A/ADORA2B gene.

In some embodiments, at least 50% of the engineered cells of a population of cells do not express a detectable level of A2a/A2b protein. For example, at least 55%, at least 60%, at least 70%, at least 75%, at least 80%, at least 85%, at least 90%, or at least 95% of the engineered cells of a population may not express a detectable level of A2a/A2b protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of A2a/A2b protein.

In some embodiments, less than 50% of the engineered cells of a population of cells express a detectable level of A2a/A2b protein. In some embodiments, less than 30% of the engineered cells of a population of cells express a detectable level of A2a/A2b protein. For example, less than 50%, less than 30%, less than 25%, less than 20%, less than 15%, less than 10%, or less than 5% of the engineered cells of a population of cells express a detectable level of A2a/A2b protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of A2a/A2b protein.

### CD16 Gene Edits

In some embodiments, the genome of a cell described herein comprises an inserted polynucleotide encoding CD16 (NCBI Gene ID: 2214). CD16 known as FcγRIII (Fc fragment of IgG receptor IIIa), is a cluster of differentiation molecule found on the surface of natural killer cells, neutrophils, monocytes, and macrophages. Its primary function is to trigger lysis by NK cells. In some embodiments, the insertion of the CD16 gene can increase ADCC of the iNK. In some embodiments, expression of CD16 is increased in cells. In some embodiments, an iPSC comprises a knock-in CD16 gene. In some embodiments, an NK cell comprises a knock-in CD16 gene.

In some embodiments, at least 50% of the engineered cells of a population of cells express a detectable level of CD16 protein. For example, at least 55%, at least 60%, at least 70%, at least 75%, at least 80%, at least 85%, at least 90%, or at least 95% of the engineered cells of a population express a detectable level of CD16 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of CD16 protein.

In some embodiments, any of the polynucleotides encoding CD16 described herein are inserted into any safe-harbor locus described herein. In some embodiments, any of the polynucleotides encoding CD16 described herein are inserted into any CISH gene locus described herein. In some embodiments, any of the polynucleotides encoding CD16 described herein are inserted into any FAS gene locus described herein. In some embodiments, any of the polynucleotides encoding CD16 described herein are inserted into any FLI1 gene locus described herein. In some embodiments, any of the polynucleotides encoding CD16 described herein are inserted into any TGFβR gene locus described herein (e.g., TGFβR1 or TGFβR2 gene locus).

### CD64 Gene Edits

In some embodiments, the genome of a cell described herein comprises an inserted polynucleotide encoding CD64 (NCBI Gene ID: 2209). CD64 known as FcγRI, is another cluster of differentiation molecule found on the surface of NK cells, neutrophils, monocytes, and macrophages. Its primary function, like CD16, is to bind to antibodies and trigger cell lysis by NK cells. In some embodiments, the insertion of the CD64 gene can increase ADCC of the iNK. In some embodiments, expression of CD64 is increased in the cell. In some embodiments, an iPSC comprises a knock-in CD64 gene. In some embodiments, an NK cell comprises a knock-in CD64 gene.

In some embodiments, at least 50% of the engineered cells of a population of cells express a detectable level of CD64 protein. For example, at least 55%, at least 60%, at least 70%, at least 75%, at least 80%, at least 85%, at least 90%, or at least 95% of the engineered cells of a population express a detectable level of CD64 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of CD64 protein.

In some embodiments, any of the polynucleotides encoding CD64 described herein are inserted into any safe-harbor locus described herein. In some embodiments, any of the polynucleotides encoding CD64 described herein are inserted into any CISH gene locus described herein. In some embodiments, any of the polynucleotides encoding CD64 described herein are inserted into any FAS gene locus described herein. In some embodiments, any of the polynucleotides encoding CD64 described herein are inserted into any FLI1 gene locus described herein. In some embodiments, any of the polynucleotides encoding CD64 described herein are inserted into any TGFβR gene locus described herein (e.g., TGFβR1 or TGFβR2).

In some embodiments, at least 50% of the engineered cells of a population of cells express a detectable level of CD64 protein. For example, at least 55%, at least 60%, at least 70%, at least 75%, at least 80%, at least 85%, at least 90%, or at least 95% of the engineered cells of a population express a detectable level of CD64 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of CD64 protein.

In some embodiments, a polynucleotide encoding CD64 described herein is inserted into any safe-harbor locus described herein. In some embodiments, a polynucleotide encoding CD64 described herein is inserted into any CISH gene locus described herein. In some embodiments, a polynucleotide encoding CD64 described herein is inserted into any FAS gene locus described herein. In some embodiments, a polynucleotide encoding CD64 described herein is inserted into any FLI1 gene locus described herein. In some embodiments, a polynucleotide encoding CD64 described herein is inserted into any TGFβR2 gene locus described herein.

### Edits to Knock-In Chimeric Antigen Receptors

A chimeric antigen receptor (CAR) refers to an artificial immune cell receptor that is engineered to recognize and bind to an antigen expressed by tumor cells. CARs can be inserted into any cells described herein. CARs are a chimera of a signaling domain of the T-cell receptor (TCR) complex and an antigen-recognizing domain (e.g., a single chain fragment (scFv) of an antibody or other antibody fragment) (Enblad et al., Human Gene Therapy. 2015; 26 (8): 498-505). CARs have the ability to redirect cell specificity and reactivity toward a selected target in a non-MHC-restricted manner. The non-MHC-restricted antigen recognition gives cells expressing CARs the ability to recognize an antigen independent of antigen processing, thus bypassing a major mechanism of tumor escape. CARs are often referenced to by the antigen they bind. For example, a “CD30 CAR”, “CD19 CAR”, a “GPC3 CAR”, “GPR87 CAR”, “CD70 CAR”, a “CD33 CAR” and a “BCMA CAR” are CARs comprising antigen binding domains that specifically bind to CD30, CD19, GPC, GPR87 CD70, CD33 or BCMA, respectively. Accordingly, such terms are interchangeable with anti-CD30 CAR, anti-CD19 CAR, anti-GPC3 CAR, anti-GPR87 CAR, anti-CD70 CAR, anti-CD33 CAR and anti-BCMA CAR. It will be understood by those of ordinary skill in the art that a CAR that specifically binds an antigen can be referred to with either terminology.

In some embodiments, any iPSC described herein expresses a CAR. In some embodiments, any NK cell described herein expresses a CAR. In some embodiments, any HSPC described herein expresses a CAR. There are four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. Fourth-generation CARs include immune stimulatory cytokines to improve cell persistence and expansion. Cytokines for fourth-generation CARS include individually or in combination any of IL-7, IL-12, IL-15, IL-18, or IL-23. CARs, in some embodiments, contain an ectodomain, commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125 (26): 4017-4023; Kakarla and Gottschalk, Cancer J. 2014; 20 (2): 151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency.

In some embodiments, a chimeric antigen receptor is a first-generation CAR. In other embodiments, a chimeric antigen receptor is a second-generation CAR. In yet other embodiments, a chimeric antigen receptor is a third generation CAR. In some embodiments, a chimeric antigen receptor is a fourth-generation CAR.

A CAR, in some embodiments, comprises an extracellular (ecto) domain comprising an antigen binding domain (e.g., an antibody, such as an scFv), a transmembrane domain, and a cytoplasmic (endo) domain.

### Ectodomain of CARs

The ectodomain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that includes the VL and VH of immunoglobulins connected with a short linker peptide. The linker, in some embodiments, includes hydrophilic residues with stretches of glycine and serine for flexibility as well as stretches of glutamate and lysine for added solubility. A single-chain variable fragment (scFv) is not actually a fragment of an antibody, but instead is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. In some embodiments, the scFv of the present disclosure is humanized. In other embodiments, the scFv is fully human. In yet other embodiments, the scFv is a chimera (e.g., of mouse and human sequence).

In some embodiments, the scFv is an anti-BCMA scFv (binds specifically to BCMA). In some embodiments, the anti-BCA scFv comprises or consists of the nucleotide sequence of SEQ ID NO: 71. In some embodiments, the anti-BCA scFv polynucleotide has at least 90%, 91%, 92%, 93%, 94%, 95%, 96%, 97%, 98%, or 99% identity to SEQ ID NO: 71. In some embodiments, the anti-BCMA CAR comprises the amino acid sequence of SEQ ID NO: 74.

The scFv can be an anti-CD30 scFv (binds specifically to CD30). In some embodiments, anti-CD30 scFv may comprise variable domains from mouse monoclonal AC10 (e.g., Brentuximab). In other embodiments, anti-CD30 scFv may comprise variable domains from human 5F11 antibody (U.S. Pat. No. 7,387,776). In some embodiments the scFV of a CD30 CAR may comprise the nucleotide sequence of SEQ ID NO: 106, SEQ ID NO: 111, or SEQ ID NO: 115. In some embodiments, the anti-CD30 polynucleotide has at least 90%, 91%, 92%, 93%, 94%, 95%, 96%, 97%, 98%, or 99% identity to SEQ ID NO: 106, SEQ ID NO: 111, or SEQ ID NO: 115. Non-limiting examples of a CD30 CAR that may be used as provided herein may include the amino acid sequence of SEQ ID NO: 109, SEQ ID NO: 113, or SEQ ID NO: 117.

In some embodiments, the scFv is an anti-CD19 scFv (binds specifically to CD19). In some embodiments, the scFv is an anti-CD70 scFv (binds specifically to CD70). In some embodiments, the scFv is an anti-CD33 scFv (binds specifically to CD33). In some embodiments, the scFv is an anti-GPC3 scFv (binds specifically to GPC3). In some embodiments, the scFv is an anti-GPR87 scFv (binds specifically to GPR87). In some embodiments, the scFv is an anti-A33 scFv (binds specifically to A33). Other scFv proteins can be used. The ectodomain of the CAR can recognize more than one antigen, e.g., the CAR can recognize and bind CD19, CD20, and BCMA

In some embodiments, the ectodomain is the region of the CAR that is exposed to the extracellular fluid and, in some embodiments, includes a NKG2D receptor, and optionally a signal peptide, a spacer domain, and/or a hinge domain. The signal peptide can enhance the antigen specificity of CAR binding. Signal peptides can be derived from antibodies, such as, but not limited to, CD8, as well as epitope tags such as, but not limited to, GST or FLAG. Examples of signal peptides include MLLLVTSLLLCELPHPAFLLIP (SEQ ID NO: 68) and MALPVTALLLPLALLLHAARP (SEQ ID NO: 69). Other signal peptides may be used.

A spacer domain or hinge domain can be located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is an oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

### Transmembrane Domain of CARS

The transmembrane domain is a hydrophobic alpha helix that spans the membrane. The transmembrane domain provides stability of the CAR. In some embodiments, the transmembrane domain of the CAR is a CD8 transmembrane domain. In some embodiments, the transmembrane domain is a CD28 transmembrane domain. In yet other embodiments, the transmembrane domain is a chimera of a CD8 and CD28 transmembrane domain. In some embodiments, the CD8a transmembrane domain is the nucleotide of SEQ ID NO: 28. In some embodiments, the transmembrane domain is a CD8a transmembrane domain: FVPVFLPAKPTTTPAPRPPTPAPTIASQPLSLRPEACRPAAGGAVHTRGLDFACDIYIWAP LAGTCGVLLLSLVITLYCNHRNR (SEQ ID NO: 72). In some embodiments, the transmembrane domain is a CD8a transmembrane domain comprising the amino acid sequence: IYIWAPLAGTCGVLLLSLVITLY (SEQ ID NO: 73). In some embodiments, the transmembrane domain is a CD8 transmembrane domain comprising the amino acid sequence SAAAFVPVFLPAKPTTTPAPRPPTPAPTIASQPLSLRPEACRPAAGGAVHTRGLDFACDIY IWAPLAGTCGVLLLSLVITLYCNHRNR (SEQ ID NO: 122). Other transmembrane domains can be used.

In some embodiments, the transmembrane domain is selected from transmembrane domains of: NKG2D, FcYRIIIa, NKp44, NKp30, NKp46, actKIR, NKG2C, CD8a, CD28, and IL15Rb. In some embodiments, the transmembrane domain is an NKG2D transmembrane domain. In some embodiments, a CD28 transmembrane domain is used.

### Endodomain of CARs

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell after the antigen is bound. In many cases, CD3-zeta may not provide a fully competent activation signal and, thus, a co-stimulatory signaling is used. For example, CD28 and/or 4-1BB may be used with CD3-zeta (CD32) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3-zeta and CD28. In some embodiments, a CAR includes CD3-zeta and 4-1BB. In still other embodiments, a CAR includes CD3ζ, CD28, and 4-1BB. Table 81 provides examples of signaling domains derived from CD28, 4-1BB, and CD3-zeta that may be used herein.

TABLE 81
EXEMPLARY SIGNALING DOMAIN SEQUENCES

| Name | Sequence | SEQ ID NO: |
|---|---|---|
| CD28 | SKRSRLLHSDYMNMTPRRPGPTRKHYQPYAPPRDFAAYRS | 123 |
| 4-1BB | KRGRKKLLYIFKQPFMRPVQTTQEEDGCSCRFPEEEEGGCEL | 124 |
| CD3-zeta | RVKFSRSADAPAYQQGQNQLYNELNLGRREEYDVLDKRRG | 125 |
| | RDPEMGGKPRRKNPQEGLYNELQKDKMAEAYSEIGMKGER | |
| | RRGKGHDGLYQGLSTATKDTYDALHMQALPPR | |

In some embodiments, any of the CARs described herein have one, two or more intracellular signaling domains from, e.g., CD137/41 BB, DNAM-1, NKrdO, 2B4, NTBA, CRACC, CD2, CD27, one or more integrins (e.g., ITGB1, ITGB2, or ITGB3), IL-15R, IL-18R, IL-12R, IL-21 R, or IREla (e.g., any combination of signaling domains from two or more of these molecules).

Natural Killer cells express a number of transmembrane adapters providing them with signal enhancement. In some embodiments, the intracellular signaling domain of any CAR described herein comprises a transmembrane adapter. In some embodiments, the transmembrane adapter is a transmembrane adaptor from one or more of: FceRl y, CD3ζ, DAP 12, and DAP 10.

In some embodiments, a CARs described herein have one of more co-stimulatory domains. In some embodiments, a 2B4 co-stimulatory domain is used. In some embodiments, a CD3ζ intracellular signaling domain is used. In some embodiments, a DAP10 or DAP12 co-stimulatory domains are used with a CD32 intracellular signaling domain. In some embodiments, a DAP10 co-stimulatory signaling domain is used with an NKG2D transmembrane domain. In some embodiments, the transmembrane domain is from NKG2D, and the endodomain is from DAP10 and CD3ζ (e.g., as described in Chang Y H et al. Caner Res. 2013. 73 (6): 1777-86). In some embodiments, the CAR comprises an NKG2D transmembrane domain fused to 4-1BB and DAP10 signaling and/or co-stimulatory domains (e.g., as described in Guo C. et al. Mol Immunol. 2019. 114:108-113). In some embodiments, the CAR comprises a co-stimulatory domain from 2B4. In some embodiments, the CAR comprises a CD8 transmembrane domain and 4-1BB-CD3ζ signaling domains (e.g., as in a construct as described by Imai C, et al. Blood. 2005, 106 (1). 376-383).

In some embodiments, the CAR has a CD8 transmembrane domain, a 4-1BB intracellular domain, and a CD3ζ signaling domain. In some embodiments, the CAR has a CD28 transmembrane domain, a CD28 intracellular domain, and a CD3ζ signaling domain. In some embodiments, the CAR has a DAP12 transmembrane and intracellular domains. In some embodiments, the CAR has a 2B4 transmembrane and intracellular domains and a CD3ζ signaling domain. In some embodiments, the CAR has a CD8 transmembrane domain, a 2B4 intracellular domain, and a CD3ζ signaling domain. In some embodiments, the CAR has a CD28 transmembrane and intracellular domains, a 4-1BB intracellular domain, and a CD3ζ signaling domain. In some embodiments, the CAR has a CD16 transmembrane domain, a 2B4 intracellular domain, and a CD3ζ signaling domain. In some embodiments, the CAR has a NKp44 transmembrane domain, a DAP10 intracellular domain, and a CD3ζ signaling domain. In some embodiments, the CAR has a NKp46 transmembrane domain, a2B4 intracellular domain, and a CD3ζ signaling domain. In some embodiments, the CAR has a NKG2D transmembrane domain, a 4-1BB intracellular domain, and a CD3ζ signaling domain. In some embodiments, the CAR has a NKG2D transmembrane domain, a 4-1BB intracellular domain, and a CD3ζ signaling domain. In some embodiments, the CAR has an NKG2D transmembrane domain, a DAP12 intracellular domain, a 2B4 intracellular domain, and a CD3ζ signaling domain. In some embodiments, the CAR has an NKG2D transmembrane domain, a DAP10 intracellular domain, a 2B4 intracellular domain, and a CD3ζ signaling domain. In some embodiments, the CAR has an NKG2D transmembrane domain, a 4-1BB intracellular domain, a 2B4 intracellular domain, and a CD3ζ signaling domain. In some embodiments, the CAR has an NKG2D transmembrane domain and a CD3ζ signaling domain.

### Multi-Gene Editing

In some embodiments, the engineered cells disclosed herein include more than one gene edit, for example, in more than one gene. In some embodiments, two, three, four, five, six or more genes are edited. In some embodiments, the gene-edit is an insertion (KI). In some embodiments, the gene-edit is a disruption (KO). In some embodiments, the combination of two or more gene edits described herein is a combination of KI and KO. In some embodiments, the gene-edits are any combination of one, two, three, four, five, six or more of the gene-edits selected from: B2M KO, IL15 KI, IL15Rα KI, an IL15/IL15Rα fusion protein KI, SERPINB9 KI, GPC3 CAR KI, GPR87 CAR KI, HLA-E KI, CIITA KO, CD16 KI, CD64 KI, NKG2D CAR KI, CISH KO, FAS KO, CD38 KO, FLI1 KO, TGFβR1 KO, TGFβR2 KO, ZEB1 KO, ADORA2A/ADORA2B KO, ADAM17 KO, BCMA CAR KI, CD30 CAR KI, REGNASE-1 KO, TIGIT KO, PD-1 KO, NKG2A KO, CD70 KO, ALK4 KO (e.g., a conditional KO), A33 CAR KI, CD70 CAR KI, CD19 CAR KI, CD33 CAR KI, CD19-CD20-BCMA CAR KI, NKp30 CAR KI, CD73 CAR KI, and SLC7A11 (xCT) CAR KI. In some embodiments, the editing of two or more genes is simultaneous, such as in the same method step. For example, an engineered cell can comprise a disrupted CIITA gene, a disrupted B2M gene, a disrupted CISH gene, a disrupted FAS gene, or a combination thereof. In some embodiments the engineered cell comprises a disrupted CIITA gene, a disrupted B2M gene, a disrupted CISH gene, a disrupted FAS gene, a disrupted CD38 gene, a disrupted FLI1 gene, a disrupted TGFβR1 gene, a disrupted TGFβR2 gene, or a combination thereof.

In some embodiments, the engineered cells comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; and an insertion of a polynucleotide encoding a CAR and a polynucleotide encoding HLA-E in the disrupted CIITA gene, wherein the CAR is an anti-GPC3 CAR or an anti-GPR87 CAR; wherein the cell expresses SERPINB9, the IL15/IL15Rα fusion protein, HLA-E, and the CAR, and the cell has disrupted expressions of B2M and CIITA.

The engineered cell can comprise a disrupted FAS gene. The cell has disrupted expression of FAS. The engineered cell can comprise a disrupted CISH gene. The engineered cells can comprise: an insertion of a polynucleotide encoding CD64 in the disrupted CISH gene. In some embodiments, the cell expresses CD64 and has disrupted expression of CISH.

The engineered cells can comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; an insertion of a polynucleotide encoding an anti-GPR87 CAR and a polynucleotide encoding HLA-E in the disrupted CIITA gene; a disrupted CISH gene; and an insertion of a polynucleotide encoding CD64 in the disrupted CISH gene. In some embodiments, the cell expresses SERPINB9, the IL15/IL15Rα fusion protein, HLA-E, the anti-GPR87 CAR, and CD64. In some embodiments, the cell has a disrupted expressions of B2M, CIITA, and CISH.

In some embodiments, the engineered cells comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; an insertion of a polynucleotide encoding an anti-GPR87 CAR and a polynucleotide encoding HLA-E in the disrupted CIITA gene; a disrupted CISH gene; an insertion of a polynucleotide encoding CD64 in the disrupted CISH gene; and a disrupted FAS gene. In some embodiments, the cell expresses SERPINB9, the IL15/IL15Rα fusion protein, HLA-E, the anti-GPR87 CAR, and CD64. In some embodiments, the cell has disrupted expressions of B2M, CIITA, CISH, and FAS.

The engineered cells can comprise: a disrupted B2M gene; an insertion of a polynucleotide encoding SERPINB9 and a polynucleotide encoding a fusion of IL15 and IL15Rα (IL15/IL15Rα) in the disrupted B2M gene; a disrupted CIITA gene; an insertion of a polynucleotide encoding an anti-GPC3 CAR and a polynucleotide encoding HLA-E in the disrupted CIITA gene; a disrupted CISH gene; an insertion of a polynucleotide encoding CD64 in the disrupted CISH gene; and a disrupted FAS gene. In some embodiments, the cell expresses SERPINB9, the IL15/IL15Rα fusion protein, HLA-E, the anti-GPC3 CAR, and CD64. In some embodiments, the cell has disrupted expressions of B2M, CIITA, CISH, and FAS. The engineered cell can comprise a disrupted TGFβR2 gene. In some embodiments, the cell has disrupted expression of TGFβR2.

The polynucleotide encoding SERPINB9 can comprise the sequence of SEQ ID NO: 129 and/or the polynucleotide encoding the IL15/IL15Rα fusion protein can comprise the sequence of SEQ ID NO: 76. The polynucleotide encoding the anti-GPC3 can comprise the sequence of SEQ ID NO: 152 and/or anti-GPR87 CAR can comprise the sequence of SEQ ID NO: 159. The polynucleotide encoding HLA-E can comprise the sequence of SEQ ID NO: 51. The polynucleotide encoding HLA-E can comprise a sequence encoding an HLA-E trimer comprising the sequence of SEQ ID NO: 75. The polynucleotide encoding CD64 can comprise the sequence of SEQ ID NO: 146.

In some embodiments, a polynucleotide described herein is linked to a promoter, for example an exogenous promoter. In some embodiments, the promoter is selected from but not limited to CAGGS, CMV, EF1α, PGK, CAG, UBC, or other constitutive, inducible, temporal-, tissue-, and cell type-specific promoter.

In some embodiments, the genome-engineered cells comprise introduced or increased expression in at least one of a CAR, HLA-E, IL15, IL15Rα, an IL15/IL15Rα fusion protein and SERPINB9. In some embodiments, any genome-engineered cell is HLA class I and/or class II deficient. In some embodiments, the genome-engineered cells comprise integrated or non-integrated exogenous polynucleotide encoding one or more of IL15, IL15Rα, an IL15/IL15Rα fusion protein and SERPINB9 proteins. In some embodiments, the genome-engineered cells comprise integrated or non-integrated exogenous polynucleotide encoding one or more of any of the CARs disclosed herein and HLA-E. In some embodiments, said introduced expression is an increased expression from either non-expressed or lowly expressed genes comprised in said cells. In some embodiments, the non-integrated exogenous polynucleotides are introduced using Sendai virus, AAV, episomal, or plasmid. In some embodiments, the cells are B2M null, with introduced expression of IL15, IL15Rα (or an IL15/IL15Rα fusion protein) and SERPINB9. In some embodiments, the cells are HLA-A, HLA-B, and HLA-C null, with introduced expression of HLA-E. In some embodiments, the cells are CIITA null, with introduced expression of any CAR disclosed here in and HLA-E. Methods of generating any of the genetically modified cells described herein are contemplated to be performed using but not limited to, any of the gene editing methods described herein.

In some embodiments, a polynucleotide encoding HLA-E is inserted at a site within or near a CIITA gene locus in any cell described herein. In some embodiments, a polynucleotide encoding HLA-E is inserted at a site within or near a CIITA gene locus concurrent with, or following a deletion of, all or part of a CIITA gene or promoter. In some embodiments, the polynucleotide encoding HLA-E is operably linked to an exogenous promoter. In some embodiments, the polynucleotide encoding HLA-E is operably linked to the CAGGS promoter. In some embodiments, any cell described herein is gene edited to express a polynucleotide encoding HLA-E operably linked to the CAGGS promoter.

In some embodiments, a polynucleotide encoding IL15/IL15Rα fusion protein is inserted at a site within or near a B2M gene locus in any cell described herein. In some embodiments, a polynucleotide encoding IL15/IL15Rα fusion is inserted at a site within or near a B2M gene locus concurrent with, or following a deletion of, all or part of a B2M gene or promoter. In some embodiments, the polynucleotide encoding IL15/IL15Rα fusion is operably linked to an exogenous promoter. In some embodiments, the polynucleotide encoding IL15/IL15Rα fusion is operably linked to the CAGGS promoter. In some embodiments, any cell described herein is gene edited to express a polynucleotide encoding IL15/IL15Rα fusion operably linked to the CAGGS promoter.

In some embodiments, a polynucleotide encoding SERPINB9 protein is inserted at a site within or near a B2M gene locus in any cell described herein. In some embodiments, a polynucleotide encoding SERPINB9 is inserted at a site within or near a B2M gene locus concurrent with, or following a deletion of, all or part of a B2M gene or promoter. In some embodiments, the polynucleotide encoding SERPINB9 is operably linked to an exogenous promoter. In some embodiments, the polynucleotide encoding SERPINB9 is operably linked to the CAGGS promoter. In some embodiments, any cell described herein is gene edited to express a polynucleotide encoding SERPINB9 operably linked to the CAGGS promoter.

In some embodiments, a polynucleotide encoding SERPINB9-P2A-IL15/IL15Rα is inserted at a site within or near a B2M gene locus in any cell described herein. In some embodiments, the SERPINB9-P2A-IL15/IL15Rα donor plasmid (SEQ ID NO: 136) is electroporated into any cell described herein along with the ribonucleoprotein (RNP) complex made of up of B2M targeting gRNA (comprising a spacer sequence corresponding to a sequence of SEQ ID NOs: 34, 78, or 79) and Cas9 protein to yield a B2M null, SERPINB9-P2A-IL15/IL15Rα expressing cell.

In some embodiments, the edited cells described herein express at least one chimeric antigen receptor (CAR). In some embodiments, the CAR is inserted at a specific gene locus. In some embodiments, the CAR is inserted at a specific locus to simultaneously disrupt expression of a target gene. In some embodiments, a polynucleotide encoding any CAR described herein is inserted within or near a CIITA gene locus. In some embodiments, a polynucleotide encoding any CAR described herein is inserted within or near a CIITA gene locus concurrent with, or following a disruption of CIITA. In some embodiments, a polynucleotide encoding a GPC3 CAR is inserted within or near a CIITA gene locus concurrent with, or following a disruption of CIITA. In some embodiments, a polynucleotide encoding GPC3 CAR-P2A-HLA-E is inserted within or near a CIITA gene locus concurrent with, or following a disruption of CIITA. In some embodiments, a polynucleotide encoding a GPR87 CAR is inserted within or near a CIITA gene locus concurrent with, or following a disruption of CIITA. In some embodiments, a polynucleotide encoding GPR87 CAR-P2A-HLA-E is inserted within or near a CIITA gene locus concurrent with, or following a disruption of CIITA.

In some embodiments, a donor plasmid comprising the polynucleotide encoding anti-GCP3 CAR, anti-GPR87, anti-GPC3 CAR-P2A-HLA-E, or anti-GPR87 CAR-P2A-HLA-E is electroporated (or otherwise transformed using methods known in the art) into any cell described herein along with the ribonucleoprotein (RNP) complex comprising any CIITA targeting gRNA (comprising a spacer sequence corresponding to a sequence of any one of SEQ ID NOs: 13-17) and Cas9 protein to yield a CIITA null and anti-GPC3 CAR and HLA-E expressing, or anti-GPR87 CAR and HLA-E expressing cell. The donor plasmid comprises homology arms having sequence homology with genomic regions located on either side of the target site in the CIITA gene locus. The nucleotide sequence having sequence homology with a genomic region located left of the target site in the CIITA locus comprises or consists of SEQ ID NO: 22, and the nucleotide sequence having sequence homology with a genomic region located right of the target site in the CIITA gene locus comprises or consists of SEQ ID NO: 32. In some embodiments, the donor plasmid comprising the GPC3 CAR and/or the HLA-E comprising the sequence of SEQ ID NO: 156. In some embodiments, the donor plasmid comprising the GPR87 CAR and/or the HLA-E comprises or consists of the sequence of SEQ ID NO: 158.

In some embodiments, the BCMA CAR donor plasmid (SEQ ID NO: 66) is electroporated into any iPSC described herein along with the ribonucleoprotein (RNP) complex comprising a CIITA targeting gRNA (comprising a spacer sequence of SEQ ID NO: 175) and Cas9 protein to yield a CIITA null, BCMA-CAR KI expressing cell. In some embodiments, the BCMA-CAR donor plasmid (SEQ ID NO: 66) is electroporated into any cell described herein along with the ribonucleoprotein (RNP) complex made of up of any CIITA targeting gRNA (comprising a spacer sequence corresponding to a sequence of any one of SEQ ID NOs: 13-17) and Cas9 protein to yield a CIITA null, BCMA-CAR expressing cell. In some embodiments, the BCMA CAR donor plasmid (SEQ ID NO: 66) is electroporated into any iPSC described herein along with the ribonucleoprotein (RNP) complex comprising a CIITA targeting gRNA (comprising a spacer sequence of SEQ ID NO: 175) and Cas9 protein to yield a CIITA null, BCMA-CAR KI expressing cell. In some embodiments, a polynucleotide encoding a BCMA-CAR is inserted within the CIITA gene locus. In some embodiments, the polynucleotide of SEQ ID NO: 66 encoding a BCMA-CAR is inserted at a site within or near a CIITA gene locus. In some embodiments, a polynucleotide encoding BCMA-CAR is inserted at a site within or near a CIITA gene locus concurrent with or following a deletion of a CIITA gene or promoter. In some embodiments, the BCMA CAR is inserted into the CIITA gene locus wherein 86 base pairs (bp) of CIITA exon 2 are removed after homology directed repair. In some embodiments, the BCMA CAR is inserted in the CIITA gene locus using into a donor plasmid. In some embodiments, a BCMA CAR donor plasmid is electroporated into any cell described herein along with the ribonucleoprotein (RNP) complex made of up of any CIITA targeting gRNA and Cas9 protein. In some embodiments, the BCMA-CAR inserted into the CIITA gene locus is driven by any promoter described herein. In some embodiments, the BCMA-CAR inserted into the CIITA gene locus is driven by the CAG promoter. In some embodiments, any cell described herein is gene-edited to express a BCMA-CAR within the CIITA gene locus. In some embodiments, an iPSC is gene-edited to express a BCMA-CAR within the CIITA gene locus.

In some embodiments, the BCMA-CAR donor plasmid (SEQ ID NO: 66) is electroporated into any cell described herein along with the ribonucleoprotein (RNP) complex made of up of any CIITA targeting gRNA (comprising a spacer sequence corresponding to a sequence of any one of SEQ ID NOs: 13-17) and Cas9 protein to yield a CIITA null, BCMA-CAR expressing cell. In some embodiments, the BCMA CAR donor plasmid (SEQ ID NO: 66) is electroporated into any iPSC described herein along with the ribonucleoprotein (RNP) complex made of up of CIITA targeting gRNA (comprising a spacer sequence corresponding to the sequence of SEQ ID NO: 13) and Cas9 protein to yield a CIITA null, BCMA-CAR KI expressing cell.

In some embodiments, a polynucleotide encoding a CD30-CAR is inserted within the CIITA gene locus. In some embodiments, the polynucleotide of SEQ ID NO: 108, 112, or 116 encoding a CD30 CAR is inserted at a site within or near a CIITA gene locus. In some embodiments, the polynucleotide of SEQ ID NO: 119, 120, or 121 encoding a CD30 CAR-P2A-HLA-E trimer is inserted at a site within or near a CIITA gene locus. In some embodiments, a polynucleotide encoding CD30 CAR or CD30 CAR-P2A-HLA-E trimer is inserted at a site within or near a CIITA gene locus concurrent with or following a deletion of a CIITA gene or promoter. In some embodiments, the CD30 CAR or CD30 CAR-P2A-HLA-E trimer is inserted into the CIITA gene locus wherein 86 base pairs (bp) of CIITA exon 2 are removed after homology directed repair. In some embodiments, the CD30 CAR or CD30 CAR-P2A-HLA-E trimer is inserted into the CIITA gene locus using a donor plasmid. In some embodiments, a CD30 CAR or CD30 CAR-P2A-HLA-E trimer donor plasmid is electroporated into any cell described herein along with the ribonucleoprotein (RNP) complex made of up of any CIITA targeting gRNA and Cas9 protein. In some embodiments, the CD30 CAR or CD30 CAR-P2A-HLA-E trimer inserted into the CIITA gene locus is driven by any promoter described herein. In some embodiments, the CD30 CAR or CD30 CAR-P2A-HLA-E trimer inserted into the CIITA gene locus is driven by the CAG promoter. In some embodiments, any cell described herein is gene-edited to express a CD30 CAR or CD30 CAR-P2A-HLA-E trimer within the CIITA gene locus. In some embodiments, an iPSC is gene-edited to express a CD30 CAR or CD30 CAR-P2A-HLA-E trimer within the CIITA gene locus.

In some embodiments, the CD30 CAR-P2A-HLA-E trimer donor plasmid (SEQ ID NO: 110, 114, or 118) is electroporated into any cell described herein along with the ribonucleoprotein (RNP) complex made of up of any CIITA targeting gRNA (comprising a spacer sequence corresponding to a sequence of any one of SEQ ID NOs: 13-17) and Cas9 protein to yield a CIITA null, CD30 CAR, HLA-E expressing cell. In some embodiments, the CD30 CAR-P2A-HLA-E trimer donor plasmid (SEQ ID NO: 110, 114, or 118) is electroporated into any iPSC described herein along with the ribonucleoprotein (RNP) complex comprising a CIITA targeting gRNA (comprising a spacer sequence of SEQ ID NO: 175) and Cas9 protein to yield a CIITA null, CD30 CAR, HLA-E expressing cell.

The one or more edits described herein can be introduced to a genome of a cell in any order. In some embodiments, any B2M null, SERPINB9-P2A-IL15/IR15α KI cell described herein is electroporated with anti-GPC3 CAR-P2A-HLA-E or anti-GPR87 CAR-P2A-HLA-E donor plasmid (along with the ribonucleoprotein (RNP) complex made of up of CIITA targeting gRNA (comprising a spacer sequence corresponding to a sequence of any one of SEQ ID NOs: 13-17) and Cas9 protein to yield a B2M null, SERPB9 KI, IL15/IL15Rα KI, anti-GPC3 CAR or anti-GPR87 CAR KI, HLA-E KI, CIITA null expressing cell. In some embodiments, the cells are further modified to insert a polynucleotide encoding CD16 in the CISH, FAS, FLI1, TGFβR1, or TGFβR2 gene locus, insert a polynucleotide encoding CD64 the CISH, FAS, FLI1, TGFβR1, or TGFβR2 gene locus, insert a polynucleotide encoding NKG2F CAR in an alternate location of the B2M gene locus, disrupt the CD38 gene, disrupt the FLI1 gene, and/or disrupt the TGFβR2 gene.

Disclosed herein include in vitro methods for generating an engineered cell. In some embodiments, the method comprising delivering to a cell: an RNP complex comprising an RNA-guided endonuclease and a gRNA targeting a target site in a B2M gene locus or an RNA-guided endonuclease and a gRNA targeting a target site in the B2M gene locus; a vector comprising a nucleic acid, the nucleic acid comprising: (i) a nucleotide sequence encoding SERPINB9 and a nucleotide sequence encoding a fusion of IL15 and IL15Rα (IL15/IL15Rα); (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); an RNP complex comprising an RNA-guided endonuclease and a gRNA targeting a target site in a CIITA gene locus or an RNA-guided endonuclease and a gRNA targeting a target site in the CIITA gene locus; and a vector comprising a nucleic acid, the nucleic acid comprising: (i) a nucleotide sequence encoding a CAR and a nucleotide sequence encoding an HLA-E trimer, wherein the CAR is an anti-GPC3 CAR or an anti-GPR87 CAR; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); wherein the B2M gene locus is cleaved at the target site and the nucleotide sequences encoding SERPINB9 and the IL15/IL15Rα fusion protein are inserted into the B2M gene locus, thereby disrupting the B2M gene, and wherein the CIITA gene locus is cleaved at the target site and the nucleotide sequences encoding the CAR and the HLA-E trimer are inserted into the CIITA gene locus, thereby disrupting the CIITA gene.

In some embodiments, the method comprises delivering to the cell: an RNP complex comprising an RNA-guided endonuclease and a gRNA targeting a target site in a CISH gene locus or an RNA-guided endonuclease and a gRNA targeting a target site in the CISH gene locus. In some embodiments, the method comprises delivering to the cell a vector comprising a nucleic acid, the nucleic acid comprising: (i) a nucleotide sequence encoding CD64; (ii) a nucleotide sequence having sequence homology with a genomic region located left of the target site in the CISH gene locus; and (iii) a nucleotide sequence having sequence homology with a genomic region located right of the target site in the CISH gene locus, wherein (i) is flanked by (ii) and (iii), wherein the CISH gene locus is cleaved at the target site and the nucleotide sequence encoding CD64 is inserted into the CISH gene locus, thereby disrupting the CISH gene.

In some embodiments, the method comprises delivering to the cell: an RNP complex comprising an RNA-guided endonuclease and a gRNA targeting a target site in a FAS gene locus or an RNA-guided endonuclease and a gRNA targeting a target site in the FAS gene locus, wherein the FAS gene locus is cleaved at the target site, thereby disrupting the FAS gene. In some embodiments, the method comprises delivering to the cell: an RNP complex comprising an RNA-guided endonuclease and a gRNA targeting a target site in a TGFβR2 gene locus or an RNA-guided endonuclease and a gRNA targeting a target site in the TGFβR2 gene locus, wherein the TGFβR2 gene locus is cleaved at the target site, thereby disrupting the TGFβR2 gene.

In some embodiments, the RNP complex comprising the RNA-guided endonuclease and the gRNA targeting a target site in the CIITA gene locus or the RNA-guided endonuclease and the gRNA targeting a target site in the CIITA gene locus; and the vector comprising the nucleic acid comprising: (i) the nucleotide sequence encoding the CAR and the nucleotide sequence encoding the HLA-E trimer; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and (iii) are delivered to the cell after the nucleotide sequences encoding SERPINB9 and the IL15/IL15Rα fusion protein are inserted into the B2M gene locus.

In some embodiments, the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii) are delivered to the cell after the nucleotide sequences encoding SERPINB9 and the IL15/IL15Rα fusion protein are inserted into the B2M gene locus and after the nucleotide sequences encoding the CAR and the HLA-E trimer are inserted into the CIITA gene locus.

In some embodiments, the RNP complex comprising the RNA-guided endonuclease and the gRNA targeting the target site in the FAS gene locus or the RNA-guided endonuclease and the gRNA targeting the target site in the FAS gene locus are delivered to the cell after the nucleotide sequences encoding SERPINB9 and the IL15/IL15Rα fusion protein are inserted into the B2M gene locus, the nucleotide sequences encoding the CAR and the HLA-E trimer are inserted into the CIITA gene locus, and after the nucleotide sequence encoding CD64 is inserted into the CISH gene locus.

In some embodiments, the RNP complex comprising the RNA-guided endonuclease and the gRNA targeting the target site in the TGFβR2 gene locus or the RNA-guided endonuclease and the gRNA targeting the target site in the TGFβR2 gene locus are delivered to the cell after the nucleotide sequences encoding SERPINB9 and the IL15/IL15Rα fusion protein are inserted into the B2M gene locus, after the nucleotide sequences encoding the CAR and the HLA-E trimer are inserted into the CIITA gene locus, and after the nucleotide sequence encoding CD64 is inserted into the CISH gene locus; and wherein the RNP complex comprising the RNA-guided endonuclease and the gRNA targeting a target site in the FAS gene locus or the RNA-guided endonuclease and the gRNA targeting a target site in the FAS gene locus are delivered to the cell after the nucleotide sequence encoding CD64 is inserted into the CISH gene locus, and after the TGFβR2 gene locus is disrupted.

In some embodiments, the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii) are delivered to the cell after the nucleotide sequences encoding SERPINB9 and the IL15/IL15Rα fusion protein are inserted into the B2M gene locus.

In some embodiments, the RNP complex comprising the RNA-guided endonuclease and the gRNA targeting the target site in the FAS gene locus or the RNA-guided endonuclease and the gRNA targeting the target site in the FAS gene locus are delivered to the cell after the nucleotide sequences encoding SERPINB9 and the IL15/IL15Rα fusion protein are inserted into the B2M gene locus and after the nucleotide sequence encoding CD64 is inserted into the CISH gene locus.

In some embodiments, the RNP complex comprising the RNA-guided endonuclease and the gRNA targeting the target site in the CIITA gene locus or the RNA-guided endonuclease and the gRNA targeting the target site in the CIITA gene locus and the vector comprising the nucleic acid comprising: (i) the nucleotide sequence encoding the CAR and the nucleotide sequence encoding the HLA-E trimer; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and (iii) are delivered to the cell after the nucleotide sequences encoding SERPINB9 and the IL15/IL15Rα fusion protein are inserted into the B2M gene locus, after the nucleotide sequence encoding CD64 is inserted into the CISH gene locus, and after the FAS gene locus is disrupted.

The gRNA targeting the target site in the B2M gene locus can comprise a spacer sequence of any one of SEQ ID NOs: 180 and 187-188. In some embodiments, the gRNA targeting the target site in the B2M gene locus comprises a sequence differing by one, two, three, four, or five mismatches relative to any one of the sequences of SEQ ID NOs: 180 and 187-188. In some embodiments, the gRNA targeting the target site in the B2M gene locus consists of a sequence selected from the sequences of SEQ ID NOs: 180 and 187-188. The gRNA targeting the target site in the B2M gene locus can comprise or consist of a spacer sequence of SEQ ID NO: 180.

The gRNA targeting the target site in the CIITA gene locus can comprise a spacer sequence of any one of SEQ ID NOs: 175-179. In some embodiments, the gRNA targeting the target site in the CIITA gene locus comprises a sequence differing by one, two, three, four, or five mismatches relative to any one of the sequences of SEQ ID NOs: 175-179. In some embodiments, the gRNA targeting the target site in the CIITA gene locus consists of a sequence selected from the sequences of SEQ ID NOs: 175-179. The gRNA targeting the target site in the CIITA gene locus can comprise or consist of a spacer sequence of SEQ ID NO: 175.

The gRNA targeting the target site in the CISH gene locus can comprise a spacer sequence of any one of SEQ ID NOs: 190-201. In some embodiments, the gRNA targeting the target site in the CISH gene locus comprises a sequence differing by one, two, three, four, or five mismatches relative to any one of the sequences of SEQ ID NOs: 190-201. In some embodiments, the gRNA targeting the target site in the CISH gene locus consists of a sequence selected from the sequences of SEQ ID NOs: 190-201. The gRNA targeting the target site in the CISH gene locus can comprise or consist of a spacer sequence of SEQ ID NO: 191.

The gRNA targeting the target site in the FAS gene locus can comprise a spacer sequence of any one of SEQ ID NOs: 181-186 and 189. In some embodiments, the gRNA targeting the target site in the FAS gene locus comprises a sequence differing by one, two, three, four, or five mismatches relative to any one of the sequences of SEQ ID NOs: 181-186 and 189. In some embodiments, the gRNA targeting the target site in the FAS gene locus consists of a sequence selected from the sequences of SEQ ID NOs: 181-186 and 189. The gRNA targeting the target site in the FAS gene locus can comprise or consist of a spacer sequence of SEQ ID NO: 181 or SEQ ID NO: 182.

The gRNA targeting the target site in the TGFβR2 gene locus can comprise a spacer sequence of SEQ ID NO: 211. In some embodiments, the gRNA targeting the target site in the TGFβR2 gene locus comprises a sequence differing by one, two, three, four, or five mismatches relative to any one of the sequences of SEQ ID NO: 211. In some embodiments, the gRNA targeting the target site in the TGFβR2 gene locus consists of a sequence selected from the sequences of SEQ ID NO: 211. The gRNA targeting the target site in the TGFβR2 gene locus can comprise or consist of a spacer sequence of SEQ ID NO: 211.

The nucleotide sequence having sequence homology with a genomic region located left of the target site in the B2M gene locus can comprise the sequence of SEQ ID NO: 36 and the nucleotide sequence having sequence homology with a genomic region located right of the target site in the B2M gene locus can comprise the sequence of SEQ ID NO: 54. The nucleotide sequence having sequence homology with a genomic region located left of the target site in the B2M gene locus can comprise the sequence of SEQ ID NO: 36, or a polynucleotide sequence having at least 85%, 90%, 95%, or 99% sequence identity to SEQ ID NO: 36 and the nucleotide sequence having sequence homology with a genomic region located right of the target site in the B2M gene locus can comprise the sequence of SEQ ID NO: 54, or a polynucleotide sequence having at least 85%, 90%, 95%, or 99% sequence identity to SEQ ID NO: 54.

The nucleotide sequence having sequence homology with a genomic region located left of the target site in the CIITA gene locus can comprise the sequence of SEQ ID NO: 22 and the nucleotide sequence having sequence homology with a genomic region located right of the target site in the CIITA gene locus can comprise the sequence of SEQ ID NO: 32. The nucleotide sequence having sequence homology with a genomic region located left of the target site in the CIITA gene locus can comprise the sequence of SEQ ID NO: 22, or a polynucleotide sequence having at least 85%, 90%, 95%, or 99% sequence identity to SEQ ID NO: 22 and the nucleotide sequence having sequence homology with a genomic region located right of the target site in the CIITA gene locus can comprise the sequence of SEQ ID NO: 32, or a polynucleotide sequence having at least 85%, 90%, 95%, or 99% sequence identity to SEQ ID NO: 32.

The nucleotide sequence having sequence homology with a genomic region located left of the target site in the CISH gene locus can comprise the sequence of SEQ ID NO: 145 and nucleotide sequence having sequence homology with a genomic region located right of the target site in the CISH gene locus can comprise the sequence of SEQ ID NO: 148. The nucleotide sequence having sequence homology with a genomic region located left of the target site in the CISH gene locus can comprise the sequence of SEQ ID NO: 145, or a polynucleotide sequence having at least 85%, 90%, 95%, or 99% sequence identity to SEQ ID NO: 145 and nucleotide sequence having sequence homology with a genomic region located right of the target site in the CISH gene locus can comprise the sequence of SEQ ID NO: 148, or a polynucleotide sequence having at least 85%, 90%, 95%, or 99% sequence identity to SEQ ID NO: 148.

The nucleotide sequence encoding SERPINB9 can comprise the sequence of SEQ ID NO: 129 (or a polynucleotide sequence having at least 85%, 90%, 95%, or 99% sequence identity to SEQ ID NO: 129) and the nucleotide sequence encoding the IL15/IL15Rα fusion protein can comprise the sequence of SEQ ID NO: 76 (or a polynucleotide sequence having at least 85%, 90%, 95%, or 99% sequence identity to SEQ ID NO: 76).

The nucleotide sequence encoding the anti-GPC3 CAR can comprise the sequence of SEQ ID NO: 152, or a polynucleotide sequence having at least 85%, 90%, 95%, or 99% sequence identity to SEQ ID NO: 152. In some embodiments, the nucleotide sequence encoding the anti-GPC3 CAR consists of the sequence of SEQ ID NO: 152. The nucleotide sequence encoding the anti-GPR87 CAR can comprise the sequence of SEQ ID NO: 159, or a polynucleotide sequence having at least 85%, 90%, 95%, or 99% sequence identity to SEQ ID NO: 159. In some embodiments, the nucleotide sequence encoding the anti-GPR87 CAR consists of the sequence of SEQ ID NO: 159. The nucleotide sequence encoding the HLA-E trimer can comprise the sequence of SEQ ID NO: 75, or a polynucleotide sequence having at least 85%, 90%, 95%, or 99% sequence identity to SEQ ID NO: 75. In some embodiments, the nucleotide sequence encoding the HLA-E trimer consists of the sequence of SEQ ID NO: 75. The nucleotide sequence encoding CD64 can comprise the sequence of SEQ ID NO: 146, or a polynucleotide sequence having at least 85%, 90%, 95%, or 99% sequence identity to SEQ ID NO: 146. In some embodiments, the nucleotide sequence encoding CD64 consists of the sequence of SEQ ID NO: 146.

In some embodiments, the engineered cells of the disclosure (e.g., cells generated by the gene editing methods disclosed herein) have disrupted expression of the gene products (e.g. a protein encoded by) of one or more of the B2M gene locus, the CIITA gene locus, the CISH gene locus, the FAS gene locus, and the TGFβR2 gene locus. In some embodiments, the disrupted expression comprises reduced or eliminated expression as compared to, e.g., unedited cells. In some embodiments, the disrupted expression comprises about, at least, or at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more, reduction in expression as compared to, e.g., an unedited cell. In some embodiments, a population of edited cells exhibits disrupted expression of a gene product one or more of the B2M gene locus, the CIITA gene locus, the CISH gene locus, the FAS gene locus, and the TGFβR2 gene locus the disrupted expression comprises about, at least, or at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more, reduction in expression as compared to, e.g., a population of unedited cells.

In some embodiments, the engineered cells as disclosed herein exhibit reduced or eliminated expression of B2M mRNA or protein (as compared to, e.g., an unedited cell). In some embodiments, the engineered cells as disclosed herein exhibit reduced or eliminated expression of B2M mRNA or protein (as compared to, e.g., an unedited cell). In some embodiments, the engineered cells as disclosed herein exhibit reduced or eliminated expression of CIITA mRNA or protein (as compared to, e.g., an unedited cell). In some embodiments, the engineered cells as disclosed herein exhibit reduced or eliminated expression of CIITA mRNA or protein (as compared to, e.g., an unedited cell). In some embodiments, the engineered cells as disclosed herein exhibit reduced or eliminated expression of CISH mRNA or protein (as compared to, e.g., an unedited cell). In some embodiments, the engineered cells as disclosed herein exhibit reduced or eliminated expression of CISH mRNA or protein (as compared to, e.g., an unedited cell). In some embodiments, the engineered cells as disclosed herein exhibit reduced or eliminated expression of FAS mRNA or protein (as compared to, e.g., an unedited cell). In some embodiments, the engineered cells as disclosed herein exhibit reduced or eliminated expression of FAS mRNA or protein (as compared to, e.g., an unedited cell). In some embodiments, the engineered cells as disclosed herein exhibit reduced or eliminated expression of TGFβR2 mRNA or protein (as compared to, e.g., an unedited cell).

In some embodiments, the engineered cells of the disclosure (e.g., cells generated by the gene editing methods disclosed herein) have increased expression of the mRNA and/or protein encoded by any of the donor polynucleotides disclosed herein. In some embodiments, the engineered cells exhibit increased expression of one or more of SERPINB9, a fusion of IL-15 and IL-15 receptor a (IL15/IL15Rα), HLA-E (e.g., an HLA-E trimer), CD64, and a CAR. In some embodiments, the increased expression comprises increased expression as compared to, e.g., unedited cells. In some embodiments, the increased expression comprises about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more increased expression as compared to, e.g., an unedited cell. In some embodiments, the engineered cells of the disclosure (e.g., cells generated by the gene editing methods disclosed herein) have increased expression of the mRNA and/or protein encoded by any of the donor polynucleotides enclosed herein. In some embodiments, a population of cell comprising the engineered cells of the disclosure exhibit increased expression of one or more of SERPINB9, a fusion of IL-15 and IL-15 receptor a (IL15/IL15Rα), HLA-E (e.g., an HLA-E trimer), CD64, and a CAR. In some embodiments, the increased expression comprises increased expression as compared to, e.g., a population of unedited cells. In some embodiments, the increased expression comprises about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more increased expression as compared to, e.g., a population of cells comprising unedited cells.

### Genome Editing Methods

Genome editing generally refers to the process of modifying the nucleotide sequence of a genome, preferably in a precise or pre-determined manner. In some embodiments, genome editing methods as described herein, e.g., the CRISPR-endonuclease system, are used to genetically modify a cell as described herein, e.g., to create a gene-edited iPSC. In some embodiments, genome editing methods as described herein, e.g., the CRISPR-endonuclease system, are used to genetically modify a cell as described herein, e.g., to introduce at least one genetic modification within or near at least one gene that increases the expression of one or more MHC-I and/or MHC-II human leukocyte antigens or other components of the MHC-I or MHC-II complex relative to an unmodified cell; to introduce at least one genetic modification that increases the expression of at least one polynucleotide that encodes a tolerogenic factor relative to an unmodified cell; and/or introduce at least one genetic modification that increases or decreases the expression of at least one gene that encodes a targeting factor that improves immunogenicity.

Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as described in Cox et al., “Therapeutic genome editing: prospects and challenges,”, Nature Medicine, 2015, 21 (2), 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature, 2015, 518, 174-76; Kent et al., Nature Structural and Molecular Biology, 2015, 22 (3): 230-7; Mateos-Gomez et al., Nature, 2015, 518, 254-57; Ceccaldi et al., Nature, 2015, 528, 258-62. In some instances, it may be possible to predict likely repair outcomes based on analysis of potential microhomologies at the site of the DNA break.

Each of these genome editing mechanisms can be used to create desired genetic modifications. A step in the genome editing process can be to create one or two DNA breaks, the latter as double-strand breaks or as two single-stranded breaks, in the target locus as near the site of intended mutation. This can be achieved via the use of endonucleases, as described herein.

### CRISPR Endonuclease System

The CRISPR-endonuclease system is a naturally occurring defense mechanism in prokaryotes that has been repurposed as an RNA-guided DNA-targeting platform used for gene editing. CRISPR systems include Types I, II, III, IV, V, and VI systems. In some aspects, the CRISPR system is a Type II CRISPR/Cas9 system. In other aspects, the CRISPR system is a Type V CRISPR/Cprf system. CRISPR systems rely on a DNA endonuclease, e.g., Cas9, and two noncoding RNAs, crisprRNA (crRNA) and trans-activating RNA (tracrRNA), to target the cleavage of DNA.

The crRNA drives sequence recognition and specificity of the CRISPR-endonuclease complex through Watson-Crick base pairing, typically with a ~20 nucleotide (nt) sequence in the target DNA. Changing the sequence of the 5′ 20 nt in the crRNA (e.g., the spacer sequence) allows targeting of the CRISPR-endonuclease complex to specific loci. The CRISPR-endonuclease complex only binds DNA sequences that contain a sequence match to the first 20 nt of the single-guide RNA (sgRNA) if the target sequence is followed by a specific short DNA motif (with the sequence NGG for some Cas proteins) referred to as a protospacer adjacent motif (PAM). TracrRNA hybridizes with the 3′ end of crRNA to form an RNA-duplex structure that is bound by the endonuclease to form the catalytically active CRISPR-endonuclease complex, which can then cleave the target DNA. Once the CRISPR-endonuclease complex is bound to DNA at a target site, two independent nuclease domains within the endonuclease each cleave one of the DNA strands three bases upstream of the PAM site, leaving a double-strand break (DSB) where both strands of the DNA terminate in a base pair (a blunt end).

The endonuclease can be a Cas9 (CRISPR associated protein 9). The Cas9 endonuclease can be, for example, Cas9 from Streptococcus pyogenes, S. aureus Cas9, N. meningitidis Cas9, S. thermophilus CRISPR 1 Cas9, S. thermophilus CRISPR 3 Cas9, or T. denticola Cas9. In some embodiments, the CRISPR endonuclease is Cpf1, e.g., L. bacterium ND2006 Cpf1 or Acidaminococcus sp. BV3L6 Cpf1. In some embodiments, the endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of the preceding endonucleases may be used. The CRISPR nuclease can be linked to at least one nuclear localization signal (NLS). The at least one NLS can be located at or within 50 amino acids of the amino-terminus of the CRISPR nuclease and/or at least one NLS can be located at or within 50 amino acids of the carboxy-terminus of the CRISPR nuclease.

Exemplary CRISPR/Cas polypeptides include the Cas9 polypeptides as published in Fonfara et al., “Phylogeny of Cas9 determines functional exchangeability of dual-RNA and Cas9 among orthologous type II CRISPR-Cas systems,” Nucleic Acids Research, 2014, 42:2577-2590. The CRISPR/Cas gene naming system has undergone extensive rewriting since the Cas genes were discovered. Fonfara et al. also provides PAM sequences for the Cas9 polypeptides from various species.

### Zinc Finger Nucleases

Zinc finger nucleases (ZFNs) are modular proteins comprised of an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZEN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96 (6): 2758-63; Dreier B et al., J Mol Biol., 2000, 303 (4): 489-502; Liu Q et al., J Biol Chem., 2002, 277 (6): 3850-6; Dreier et al., J Biol Chem., 2005, 280 (42): 35588-97; and Dreier et al., J Biol Chem. 2001, 276 (31): 29466-78.

### Transcription Activator-Like Effector Nucleases (TALENs)

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9 or CRISPR/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science, 2009 326 (5959): 1509-12; Mak et al., Science, 2012, 335 (6069): 716-9; and Moscou et al., Science, 2009, 326 (5959): 1501. The use of TALENs based on the “Golden Gate” platform, or cloning scheme, has been described by multiple groups; see, e.g., Cermak et al., Nucleic Acids Res., 2011, 39 (12): e82; Li et al., Nucleic Acids Res., 2011, 39 (14): 6315-25; Weber et al., PLOS One., 2011, 6 (2): e16765; Wang et al., J Genet Genomics, 2014, 41 (6): 339-47.; and Cermak T et al., Methods Mol Biol., 2015 1239:133-59.

### Homing Endonucleases

Homing endonucleases (HEs) are sequence-specific endonucleases that have long recognition sequences (14-44 base pairs) and cleave DNA with high specificity, often at sites unique in the genome. There are at least six known families of HEs as classified by their structure, including GIY-YIG, His-Cis box, H-N-H, PD-(D/E)xK, and Vsr-like that are derived from a broad range of hosts, including eukaryotes, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology, 2014, 24 (8): 663-80; Belfort and Bonocora, Methods Mol Biol., 2014, 1123:1-26; and Hafez and Hausner, Genome, 2012, 55 (8): 553-69.

### MegaTAL/Tev-mTALEN/MegaTev

As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., Nucleic Acids Res., 2014, 42:2591-2601; Kleinstiver et al., G3, 2014, 4:1155-65; and Boissel and Scharenberg, Methods Mol. Biol., 2015, 1239:171-96.

In a further variation, the MegaTev architecture is the fusion of a meganuclease (Mega) with the nuclease domain derived from the GIY-YIG homing endonuclease I-TevI (Tev). The two active sites are positioned ~30 bp apart on a DNA substrate and generate two DSBs with non-compatible cohesive ends; see, e.g., Wolfs et al., Nucleic Acids Res., 2014, 42, 8816-29. It is anticipated that other combinations of existing nuclease-based approaches will evolve and be useful in achieving the targeted genome modifications described herein.

dCas9-FokI or dCpf1-Fok1 and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 24 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function-retaining only the RNA-guided DNA binding function- and instead fusing a FokI domain to the deactivated Cas9; see, e.g., Tsai et al., Nature Biotech, 2014, 32:569-76; and Guilinger et al., Nature Biotech., 2014, 32:577-82. Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### Base Editing

In some embodiments, a gene is edited in a cell using base editing. Base Editing is a technique enabling the conversion of one nucleotide into another without double-stranded breaks in the DNA. Base editing allows for conversion of a C to T, G to A, or vice versa. An example editor for cytosine includes rAPOBEC1 which is fused to a catalytically inactive form of Cas9. The Cas9 helps to bind a site of interest and the rAPOBEC1 cytidine deaminase induces the point mutation. Conversion of adenine requires a mutant transfer RNA adenosine deaminase (TadA), a Cas9 nickase, and a sgRNA, as described herein. The construct is able to introduce the site-specific mutation without introducing a strand break. In some embodiments, Base Editing is used to introduce one or more mutations in a cell described herein.

### RNA-Guided Endonucleases

The RNA-guided endonuclease systems as used herein can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary endonuclease, e.g., Cas9 from S. pyogenes, US2014/0068797 SEQ ID NO: 8 or Sapranauskas et al., Nucleic Acids Res, 39 (21): 9275-9282 (2011). The endonuclease can comprise at least 70, 75, 80, 85, 90, 95, 97, 99, or 100% identity to a wild-type endonuclease (e.g., Cas9 from S. pyogenes, supra) over 10 contiguous amino acids. The endonuclease can comprise at most: 70, 75, 80, 85, 90, 95, 97, 99, or 100% identity to a wild-type endonuclease (e.g., Cas9 from S. pyogenes, supra) over 10 contiguous amino acids. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease.

The endonuclease can comprise a modified form of a wild-type exemplary endonuclease. The modified form of the wild-type exemplary endonuclease can comprise a mutation that reduces the nucleic acid-cleaving activity of the endonuclease. The modified form of the wild-type exemplary endonuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary endonuclease (e.g., Cas9 from S. pyogenes, supra). The modified form of the endonuclease can have no substantial nucleic acid-cleaving activity. When an endonuclease is a modified form that has no substantial nucleic acid-cleaving activity, it is referred to herein as “enzymatically inactive.”

Mutations contemplated can include substitutions, additions, and deletions, or any combination thereof. The mutation can convert the mutated amino acid to alanine. The mutation can convert the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to an amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

### Guide RNAs

Provided herein include guide RNAs (gRNAs) that can direct the activities of an associated endonuclease to a specific target site within a polynucleotide. A guide RNA can comprise at least a spacer sequence that hybridizes to a target nucleic acid sequence of interest, and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease.

Exemplary guide RNAs include a spacer sequences that comprises 15-200 nucleotides wherein the gRNA targets a genome location based on the GRCh38 human genome assembly. As is understood by the person of ordinary skill in the art, each gRNA can be designed to include a spacer sequence complementary to its genomic target site or region, i.e., the “target sequence.” The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. See Jinek et al., Science, 2012, 337, 816-821 and Deltcheva et al., Nature, 2011, 471, 602-607. For example, as described herein, a gRNA can comprise an RNA spacer sequence corresponding to the target DNA sequence (i.e., the RNA space sequence is the target DNA sequence in which T is substituted for U).

The gRNA can be a double-molecule guide RNA. The gRNA can be a single-molecule guide RNA. A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, an sgRNA comprises a 20 nucleotide spacer sequence at the 5′ end of the sgRNA sequence. In some embodiments, an sgRNA comprises a less than a 20 nucleotide spacer sequence at the 5′ end of the sgRNA sequence. In some embodiments, an sgRNA comprises a more than 20 nucleotide spacer sequence at the 5′ end of the sgRNA sequence. In some embodiments, an sgRNA comprises a variable length spacer sequence with 17-30 nucleotides at the 5′ end of the sgRNA sequence. In some embodiments, an sgRNA comprises a spacer extension sequence with a length of more than 1, 5, 10, 15, 20, 25, 30, 35, 40, 45, 50, 60, 70, 80, 90, 100, 120, 140, 160, 180, or 200 nucleotides. In some embodiments, an sgRNA comprises a spacer extension sequence with a length of less than 3, 5, 10, 15, 20, 25, 30, 35, 40, 45, 50, 60, 70, 80, 90, or 100 nucleotides.

An sgRNA can comprise a spacer extension sequence that comprises another moiety (e.g., a stability control sequence, an endoribonuclease binding sequence, or a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

A sgRNA can comprise a spacer sequence that hybridizes to a sequence in a target polynucleotide. The spacer of a gRNA can interact with a target polynucleotide in a sequence-specific manner via hybridization (i.e., base pairing). The nucleotide sequence of the spacer can vary depending on the sequence of the target nucleic acid of interest. In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence.

A target polynucleotide sequence can comprise 20 nucleotides. The target polynucleotide can comprise less than 20 nucleotides. The target polynucleotide can comprise more than 20 nucleotides. The target polynucleotide can comprise at least: 5, 10, 15, 16, 17, 18, 19, 20, 21, 22, 23, 24, 25, 30 or more nucleotides. The target polynucleotide can comprise at most: 5, 10, 15, 16, 17, 18, 19, 20, 21, 22, 23, 24, 25, 30 or more nucleotides. The target polynucleotide sequence can comprise 20 bases immediately 5′ of the first nucleotide of the PAM.

A spacer sequence that hybridizes to a target polynucleotide can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides. In some examples, the spacer can comprise 18 nucleotides. In some examples, the spacer can comprise 22 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

A tracrRNA sequence can comprise nucleotides that hybridize to a minimum CRISPR repeat sequence in a cell. A minimum tracrRNA sequence and a minimum CRISPR repeat sequence may form a duplex, i.e., a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides. The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

In some embodiments, a tracrRNA may be a 3′ tracrRNA. In some embodiments, a 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, a gRNA comprises a tracrRNA extension sequence. A tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, or 200 nucleotides. The tracrRNA extension sequence can have a length from about 20 to about 5000 or more nucleotides. The tracrRNA extension sequence can have a length of less than 5, 10, 15, 20, 25, 30, 35, 40, 45, 50, 60, 70, 80, 90, or 100 nucleotides. The tracrRNA extension sequence can comprise less than 10 nucleotides in length. The tracrRNA extension sequence can be 10-30 nucleotides in length. The tracrRNA extension sequence can be 30-70 nucleotides in length. The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nt to about 100 nt, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt.

In some embodiments, an sgRNA comprise a linker sequence with a length from about 3 nt to about 100 nt. In Jinek et al., supra, for example, a simple 4 nucleotide “tetraloop” (-GAAA-) was used (Jinek et al., Science, 2012, 337 (6096): 816-821). An illustrative linker has a length from about 3 nt to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker does not comprise sequences that have extensive regions of homology with other portions of the guide RNA, which might cause intramolecular binding that could interfere with other functional regions of the guide. In Jinek et al., supra, a simple 4 nucleotide sequence-GAAA—was used (Jinek et al., Science, 2012, 337 (6096): 816-821), but numerous other sequences, including longer sequences can likewise be used. The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

In some embodiments, a sgRNA does not comprise a uracil, e.g., at the 3′end of the sgRNA sequence. In some embodiments, a sgRNA does comprise one or more uracils, e.g., at the 3′end of the sgRNA sequence. In some embodiments, an sgRNA comprises 1, 2, 3, 4, 5, 6, 7, 8, 9, or 10 uracils (U) at the 3′ end of the sgRNA sequence.

A sgRNA may be chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art. In some embodiments, a modified gRNA may comprise a modified backbone, for example, phosphorothioates, phosphotriesters, morpholinos, methyl phosphonates, short chain alkyl or cycloalkyl intersugar linkages or short chain heteroatomic or heterocyclic intersugar linkages.

Morpholino-based compounds are described in Braasch and David Corey, Biochemistry, 2002, 41 (14): 4503-4510; Genesis, 2001, Volume 30, Issue 3; Heasman, Dev. Biol., 2002, 243:209-214; Nasevicius et al., Nat. Genet., 2000, 26:216-220; Lacerra et al., Proc. Natl. Acad. Sci., 2000, 97:9591-9596.; and U.S. Pat. No. 5,034,506, issued Jul. 23, 1991. Cyclohexenyl nucleic acid oligonucleotide mimetics are described in Wang et al., J. Am. Chem. Soc., 2000, 122:8595-8602.

In some embodiments, a modified gRNA may comprise one or more substituted sugar moieties, e.g., one of the following at the 2′ position: OH, SH, SCH₃, F, OCN, OCH3, OCH3 O(CH2)n CH3, O(CH2)n NH2, or O(CH2)n CH3, where n is from 1 to about 10; C1 to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O-CH3); 2′-propoxy (2′-OCH2 CH2CH3); and 2′-fluoro (2′-F). Similar modifications may also be made at other positions on the gRNA, particularly the 3′ position of the sugar on the 3′ terminal nucleotide and the 5′ position of 5′ terminal nucleotide. In some examples, both a sugar and an internucleoside linkage, i.e., the backbone, of the nucleotide units can be replaced with novel groups.

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Kornberg, A., DNA Replication, W. H. Freeman & Co., San Francisco, pp75-77, 1980; Gebeyehu et al., Nucl. Acids Res. 1997, 15:4513. A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

### Complexes of a Genome-Targeting Nucleic Acid and an Endonuclease

A gRNA interacts with an endonuclease (e.g., a RNA-guided nuclease such as Cas9), thereby forming a complex. The gRNA guides the endonuclease to a target polynucleotide. The endonuclease and gRNA can each be administered separately to a cell or a subject. In some embodiments, the endonuclease can be pre-complexed with one or more guide RNAs, or one or more crRNA together with a tracrRNA. The pre-complexed material can then be administered to a cell or a subject. Such pre-complexed material is known as a ribonucleoprotein particle (RNP). The endonuclease in the RNP can be, for example, a Cas9 endonuclease or a Cpf1 endonuclease. The endonuclease can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The weight ratio of genome-targeting nucleic acid to endonuclease in the RNP can be 1:1. For example, the weight ratio of sgRNA to Cas9 endonuclease in the RNP can be 1:1.

### Cells

Disclosed herein include populations of cells comprising one or more engineered cells described herein. In some embodiments, the population or populations of lineage-restricted progenitor cells or fully differentiated somatic cells derived from one or more engineered cells described herein.

Cells described herein can have any of the gene-edits described herein. In some embodiments, a cell (and corresponding unmodified cell) is a mammalian cell. In some embodiments, a cell (and corresponding unmodified cell) is a human cell. In some embodiments, a cell (and corresponding unmodified cell) is a stem cell. In some embodiments, a cell (and corresponding unmodified cell) is a pluripotent stem cell (PSC). In some embodiments, a cell (and corresponding unmodified cell) is an embryonic stem cell (ESC), an adult stem cell (ASC), an induced pluripotent stem cell (iPSC), or a hematopoietic stem or progenitor cell (HSPC). In some embodiments, a cell is an iPSC. In some embodiments, a cell may be a differentiated cell. In some embodiments, a cell is a somatic cell, e.g., an immune system cell or a contractile cell, e.g., a skeletal muscle cell. In some embodiments, the stem cells described herein (e.g., iPSCs) are gene-edited as described herein and then differentiated into a cell type of interest. In some embodiments, the differentiated cell retains the gene-edits of the cell from which it is derived.

The cells described herein may be differentiated into relevant cell types. In general, differentiation comprises maintaining the cells of interest for a period time and under conditions sufficient for the cells to differentiate into the differentiated cells of interest. For example, the engineered stem cells disclosed herein may be differentiated into mesenchymal progenitor cells (MPCs), hypoimmunogenic cardiomyocytes, muscle progenitor cells, blast cells, endothelial cells (ECs), macrophages, natural killer cells, hepatocytes, beta cells (e.g., pancreatic beta cells), pancreatic endoderm progenitors, pancreatic endocrine progenitors, or neural progenitor cells (NPCs). In some embodiments, any of the stem cells described herein are differentiated after gene-editing. In some embodiments, a cell is differentiated into a natural killer (NK) cell.

Stem cells are capable of both proliferation and giving rise to more progenitor cells, these in turn having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell that itself is derived from a multipotent cell, and so on. While each of these multipotent cells may be considered stem cells, the range of cell types that each can give rise to may vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required for “stem-ness.”

A “differentiated cell” is a cell that has progressed further down the developmental pathway than the cell to which it is being compared. Thus, stem cells can differentiate into lineage-restricted precursor cells (such as a hematopoietic stem and progenitor cell (HSPC)), which in turn can differentiate into other types of precursor cells further down the pathway (such as a common lymphoid progenitor cell), and then to an end-stage differentiated cell, such as a natural killer cell, which plays a characteristic role in a certain tissue type, and may or may not retain the capacity to proliferate further.

In some embodiments, any of the gene-edited cells described herein have one of more of the following characteristics: increased persistency, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein have one of more of the following characteristics relative to an un-edited (wild-type) cell described herein; increased persistency, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein are capable of cell expansion in the absence of exogenous IL15.

### Embryonic Stem Cells

The cells described herein may be embryonic stem cells (ESCs). ESCs are derived from blastocysts or other structures of mammalian embryos and are able differentiate into any cell type and propagate rapidly. ESCs typically maintain high telomerase activity and exhibit remarkable long-term proliferative potential, making these cells excellent candidates for use as gene-edited stem cells. In some embodiments, ESCs with one, two, three, four, five, six or more, or all, of the following edits: B2M null, CIITA null, ADAM17 null, HLA-E knock-in, IL15 knock-in, IL15Rα knock-in, BCMA CAR knock-in, CD30 CAR knock-in, GPC3 CAR knock-in, GPR87 CAR knock-in, SERPINB9 knock-in, FAS null, CISH null, and REGNASE-1 null, are differentiated into NK cells.

### Adult Stem Cells

The cells described herein may be adult stem cells (ASCs). ASCs are undifferentiated cells that may be found in mammals, e.g., humans. ASCs are defined by their ability to self-renew, e.g., be passaged through several rounds of cell replication while maintaining their undifferentiated state, and ability to differentiate into several distinct cell types, e.g., glial cells. Adult stem cells are a broad class of stem cells that may encompass hematopoietic stem cells, mammary stem cells, intestinal stem cells, mesenchymal stem cells, endothelial stem cells, neural stem cells, olfactory adult stem cells, neural crest stem cells, and testicular cells. In some embodiments, ASCs with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

### Induced Pluripotent Stem Cells

The cells described herein may be induced pluripotent stem cells (iPSCs). An iPSC may be generated directly from an adult human cell by introducing genes that encode critical transcription factors involved in pluripotency, e.g., Oct4, Sox2, cMyc, and Klf4. An iPSC may be derived from the same subject to which subsequent progenitor cells are to be administered. That is, a somatic cell can be obtained from a subject, reprogrammed to an induced pluripotent stem cell, and then re-differentiated into a progenitor cell to be administered to the subject (e.g., autologous cells). However, in the case of autologous cells, a risk of immune response and poor viability post-engraftment remain. In some embodiments, iPSC are generated from adult somatic cells using genetic reprogramming methods known in the art. In some embodiments, the iPSCs are derived from a commercial source. In some embodiments, the cells described herein are iPSCs or a derivative cell. In some embodiments, iPSC with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

### Mesoderm

The cells described herein can be mesodermal cells which is one of the three germinal layers in embryonic development. The mesoderm eventually differentiates into, but is not limited to muscle, connective tissue, bone, red blood cells, white blood cells, and microglia. In some embodiments, the gene-edited cells are mesodermal cells. In some embodiments, mesodermal cells are derived from any of the stem cells described herein. In some embodiments, mesodermal cells are derived from iPSC. In some embodiments, the mesodermal cells have any of the gene-edits described herein. In some embodiments, the mesodermal cells are differentiated into NK cells. In some embodiments, mesodermal cells with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

### Hemogenic Endothelium

The cells described herein can be hemogenic endothelium (HE) cells which is an intermediate precursor of hematopoietic progenitors. In some embodiments, the cells are hemogenic endothelium cells. In some embodiments, the gene-edited cells described herein are hemogenic endothelium cells. In some embodiments, hemogenic endothelium cells are derived from any of the stem cells described herein. In some embodiments, hemogenic endothelium cells are derived from iPSC. In some embodiments, the hemogenic endothelial cells have any of the gene-edits described herein. In some embodiments, the hemogenic endothelial cells are differentiated into NK cells. In some embodiments, HE cells with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

### Human Hematopoietic Stem and Progenitor Cells

The cells described herein can be human hematopoietic stem and progenitor cells (hHSPCs). This stem cell lineage gives rise to all blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). Blood cells are produced by the proliferation and differentiation of a very small population of pluripotent hematopoietic stem cells (HSCs) that also have the ability to replenish themselves by self-renewal. During differentiation, the progeny of HSCs progress through various intermediate maturational stages, generating multi-potential and lineage-committed progenitor cells prior to reaching maturity. Bone marrow (BM) is the major site of hematopoiesis in humans and, under normal conditions, only small numbers of hematopoietic stem and progenitor cells (HSPCs) can be found in the peripheral blood (PB). Treatment with cytokines, some myelosuppressive drugs used in cancer treatment, and compounds that disrupt the interaction between hematopoietic and BM stromal cells can rapidly mobilize large numbers of stem and progenitors into the circulation. In some embodiments, HSPCs are derived from any of the stem cells described herein. In some embodiments, HSPCs are derived from iPSCs. In some embodiments, the HSPCs have any of the gene-edits described herein. In some embodiments, the HSPCs cells are differentiated into NK cells. In some embodiments, HSPCs with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

### Common Lymphoid Progenitor

The cells described herein can be common lymphoid progenitor (CLP) cells. CLPs are descendants of HSPCs. These cells differentiate into the lymphoid lineage of blood cells. Further differentiation yields B-cell progenitor cells, Natural Killer cells, and Thymocytes. In some embodiments, the cells described herein are common lymphoid progenitors. In some embodiments, the gene-edited cells described herein are common lymphoid progenitors. In some embodiments, CLP cells are derived from iPSCs. In some embodiments, the CLP cells have any of the gene-edits described herein. In some embodiments, the CLP cells are differentiated into NK cells. In some embodiments, CLP cells with one, two, three, four, five, six or more, or all, of the following edits: B2M null, CIITA null, ADAM17 null, HLA-E knock-in, IL15 knock-in, IL15Rα knock-in, GPC3 CAR knock-in, GPR87 CAR knock-in, NKG2D CAR knock-in, CD30 CAR knock-in, SERPINB9 knock-in, FAS null, CISH null, CD38 null, FLI1 null, TGFβR1/TGFβR2 null, CD16 KI, CD64 KI, and/or REGNASE-1 null, are differentiated into NK cells.

Differentiation of Cells into Other Cell Types

Another step of the methods of the present disclosure may comprise differentiating cells into differentiated cells. The differentiating step may be performed according to any method known in the art. For example, human iPSCs are differentiated into natural killer cells using methods known in the art. In some embodiments, the differentiating step may be performed according to Zhu and Kaufman, bioRxiv 2019; dx.doi.org/10.1101/614792. A differentiated cell may be any somatic cell of a mammal, e.g., a human. In some embodiments, a somatic cell may be an endocrine secretory epithelial cell (e.g., thyroid hormone secreting cells, adrenal cortical cells), an exocrine secretory epithelial cell (e.g., salivary gland mucous cell, prostate gland cell), a hormone-secreting cell (e.g., anterior pituitary cell, pancreatic islet cell), a keratinizing epithelial cell (e.g., epidermal keratinocyte), a wet stratified barrier epithelial cell, a sensory transducer cell (e.g., a photoreceptor), an autonomic neuron cells, a sense organ and peripheral neuron supporting cell (e.g., Schwann cell), a central nervous system neuron, a glial cell (e.g., astrocyte, oligodendrocyte), a lens cell, an adipocyte, a kidney cell, a barrier function cell (e.g., a duct cell), an extracellular matrix cell, a contractile cell (e.g., skeletal muscle cell, heart muscle cell, smooth muscle cell), a blood cell (e.g., erythrocyte), an immune system cell (e.g., megakaryocyte, microglial cell, neutrophil, mast cell, a T cell, a B cell, a Natural Killer cell), a germ cell (e.g., spermatid), a nurse cell, or an interstitial cell. In some embodiments, any of the stem cells described herein are differentiated into NK cells. In some embodiments, any of the derivative cell types described herein are differentiated into NK cells.

Provided herein, in some embodiments, are methods for generating Natural Killer (NK) cells from stem cells. The method includes: (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, WNT C-59 and an activin/nodal inhibitor to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF for a time sufficient to generate NK cells. In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, and an activin/nodal inhibitor to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, SCF and nicotinamide for a time sufficient to generate NK cells. In some embodiments, the second medium further includes a ROCK inhibitor. In some embodiments, the ROCK inhibitor is thiazovivin. In some embodiments, the WNT pathway activator is CHIR-99021. In some embodiments, the activin/nodal inhibitor is SB-431542.

In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, and an activin/nodal inhibitor to form a cell population comprising HSPCs; (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, and SCF for a time sufficient to generate NK cells. In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO and SCF to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, and SCF for a time sufficient to generate NK cells. In some embodiments, the ROCK inhibitor is thiazovivin. In some embodiments, the ROCK inhibitor is Y27632. In some embodiments, the WNT pathway activator is CHIR-99021. In some embodiments, the activin/nodal inhibitor is SB-431542.

In some embodiments, steps (a)-(g) occurs between 20-35 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 1-3 days. In some embodiments, step (f) includes culturing for up to 7 days. In some embodiments, step (g) includes culturing for at least 6 days and up to 21-28 days total. In some embodiments, step (a) includes culturing for 16-20 hours; step (b) includes culturing for 6-10 hours; step (c) includes culturing for 2 days; step (d) includes culturing for 2 days; step (e) includes culturing for 2 days; step (f) includes culturing for 4 days; and/or step (g) includes culturing for 14-28 days.

In some embodiments, steps (a)-(h) occur between 24 and 36 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 1-3 days. In some embodiments, step (f) includes culturing for up to 7 days. In some embodiments, step (g) includes culturing for up to 6 days. In some embodiments, step (h) includes culturing for at least 6 days and up to 10-16 days total. In some embodiments, step (a) includes culturing for 16-20 hours; step (b) includes culturing for 6-10 hours; step (c) includes culturing for 2 days; step (d) includes culturing for 2 days; step (e) includes culturing for 2 days; step (f) includes culturing for 4 days; step (g) includes culturing for 6 days and/or step (h) includes culturing for 10-16 days.

In some embodiments, steps (a)-(h) occurs between 24 and 36 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 2-6 days. In some embodiments, step (f) includes culturing for up to 7 days. In some embodiments, step (g) includes culturing for up to 6 days. In some embodiments, step (h) includes culturing for at least 6 days and up to 10-16 days total. In some embodiments, step (a) includes culturing for 16-20 hours; step (b) includes culturing for 6-10 hours; step (c) includes culturing for 2 days; step (d) includes culturing for 2 days; step (e) includes culturing for 6 days; step (f) includes culturing for 4 days; step (g) includes culturing for 6 days and/or step (h) includes culturing for 10-17 days.

In some embodiments, the method is carried out under suspension agitation. In some embodiments, the suspension agitation includes rotation. In some embodiments, the first and second media include StemFlex medium. In some embodiments, the third, fourth and fifth media include APEL medium. In some embodiments, the sixth medium can include APEL medium. In some embodiments, the sixth and seventh media can include DMEM/F12 medium. In some aspects, the sixth and seventh media comprise DMEM (high glucose)/F12 medium. In some embodiments, the sixth and seventh media include human serum (e.g., at the concentration of 10-20%), zinc sulfate (e.g., at a concentration of about 20-40 μM), ethanolamine (e.g., at a concentration of about 10-100 μM), b-mercaptoethanol (e.g., at a concentration of about 0.1-5 μM), glucose (e.g., at a total concentration of 2-40 mM), or any combination thereof. In some embodiments, the sixth and seventh media include human serum (e.g., at the concentration of 15%), zinc sulfate (e.g., at a concentration of about 36 or 37 μM), ethanolamine (e.g., at a concentration of about 50 μM), b-mercaptoethanol (e.g., at a concentration of about 1 μM), glucose (e.g., at a total concentration of 27 mM), or any combination thereof. In some embodiments, the sixth and seventh media include human serum (e.g., at a concentration of about 10-40%), zinc sulfate (e.g., at a concentration of about 20-40 μM), ethanolamine (e.g., at a concentration of about 10-100 μM), glucose (e.g., at a total concentration of about 2-40 mM), or any combination thereof. In some embodiments, the sixth and seventh media include human serum (e.g., at a concentration of about 20%), zinc sulfate (e.g., at a concentration of about 37 μM), ethanolamine (e.g., at a concentration of about 50 μM), glucose (e.g., at a total concentration of about 20 mM), or any combination thereof. In some embodiments, the eighth media includes human serum (e.g., at a concentration of about 2-15%), zinc sulfate (e.g., at a concentration of about 20-40 μM), ethanolamine (e.g., at a concentration of about 10-100 μM), glucose (e.g., at a total concentration of about 2-40 mM), or any combination thereof. In some embodiments, the eighth media can include DMEM/F12 medium. In some aspects, the eighth media comprises DMEM (high glucose)/F12 medium. In some embodiments, the eighth media includes human serum (e.g., at a concentration of about 10%), zinc sulfate (e.g., at a concentration of about 37 μM), ethanolamine (e.g., at a concentration of about 50 μM), glucose (e.g., at a total concentration of about 20 mM), or any combination thereof. In any of the sixth, seventh, and eighth media provided herein, the total glucose concentration comprises glucose from all sources including glucose present in the base media and any added glucose. In each of the sixth, seventh, and eighth media provided herein, additional glucose may be added to a glucose containing base media (e.g., DMEM, F12 or DMEM (high glucose)/F12 medium) to reach the “total” glucose concentration. In some embodiments, about 10.25 mM of glucose is added to the base media of the sixth or seventh media to reach the total glucose concentration of about 27 mM. In some embodiments, about 4.66 mM of glucose is added to the base media of the sixth or seventh media to reach the total glucose concentration of about 20 mM. In some embodiments, about 2.33 mM of glucose is added to the base media of the eighth media to reach the total glucose concentration of about 20 mM. In some embodiments, the first medium includes 10 μM of the ROCK inhibitor. In some embodiments, the first medium includes 5 μM of the ROCK inhibitor. In some embodiments, the second medium includes 30 ng/mL BMP-4 and 10 μM of a ROCK inhibitor. In some embodiments, the third medium includes 30 ng/mL BMP-4, 100 ng/ml FGF2, 6 μM CHIR-99021, and 2.5-5 ng/mL Activin A. In some embodiments, the third medium includes 30 ng/mL BMP-4, 100 ng/ml FGF2, 7 μM CHIR-99021, and 2.5-5 ng/ml Activin A. In some embodiments, the third medium includes 30 ng/ml BMP-4, 100 ng/mL FGF2, 3-3.5 μM CHIR-99021, and 2.5 ng/ml Activin A.

In some embodiments, half of the third medium is added to the stem cell aggregates. In some embodiments, the fourth and fifth media include 20 ng/ml FGF, 20 ng/ml VEGF, 20 ng/mL TPO, 100 ng/mL SCF, 40 ng/ml IL-3, and 10-20 ng/mL FLT3L. In some embodiments, the fourth medium further includes 2 μM WNT C-59 and 5 μM SB-431542. In some embodiments, the fourth medium further includes 5 μM SB-431542. In some embodiments, the fourth medium does not include WNT C-59. In some embodiments, the fourth media includes 20 ng/ml FGF, 20 ng/mL VEGF, 20 ng/mL TPO, and 40 ng/mL SCF, In some embodiments, the fourth media does not comprise IL-3, FLT3L, or SB-431542. In some embodiments, the sixth and seventh media includes 20 ng/mL IL-7, 10-20 ng/mL FLT3L, 10-20 ng/ml IL-15, and 20 ng/mL SCF. In some embodiments, the sixth medium includes 5 ng/ml IL-3. In some embodiments, the eighth media includes IL-7, FLT3L, IL-15, SCF and nicotinamide. In various embodiments, the eighth medium includes 10-20 ng/ml IL-7, 5-20 ng/mL FLT3L, 10-30 ng/mL IL-15, 20-40 ng/mL SCF, and 1-15 mM nicotinamide. In various embodiments, the eighth medium includes 10 ng/ml IL-7, 7.5 ng/mL FLT3L, 15 ng/ml IL-15, 20 ng/mL SCF and 6.5 mM nicotinamide. In some embodiments, the eighth media includes IL-7, FLT3L, IL-15, and SCF. In various embodiments, the eighth medium includes 10-20 ng/mL IL-7, 5-20 ng/mL FLT3L, 10-30 ng/ml IL-15, and 20-40 ng/mL SCF. In various embodiments, the eighth medium includes 10 ng/ml IL-7, 7.5 ng/mL FLT3L, 15 ng/mL IL-15, and 20 ng/ml SCF.

In some embodiments, the HSPCs of step (d) express CD34. In some embodiments, the NK cells express CD56. In some embodiments, the NK cells express at least one activating receptor. In some embodiments, the at least one activating receptor is selected from NKp44, NKp46, CD16, KIR2DL4, and any combination thereof. In some embodiments, the NK cells express at least one inhibitory receptor. In some embodiments, the at least one inhibitory receptor is selected from CD94, NKG2A, KIR3DL2, and any combination thereof.

In some embodiments, the NK cells include at least one function associated with endogenous NK cells. In some embodiments, the at least one function includes the ability to induce cell lysis and cell death of a target cell. In some embodiments, the at least one function includes degranulation. In some embodiments, the degranulation includes release of perforin and granzyme B. The degranulation can include expression of CD107a on the cell surface of an NK cell.

In some embodiments, the population of stem cells is a population of engineered cells, such as the engineered cells generated by the methods disclosed herein. In some embodiments, the population of engineered cells is differentiated by the methods of generating Natural Killer (NK) cells from stem cells disclosed herein. In some embodiments, a plurality of Natural Killer (NK) cells is generated by the method of generating Natural Killer (NK) cells from stem cells disclosed herein. Also disclosed herein is a plurality of NK cells is for use in treating a subject in need thereof. In some embodiments, the subject is a human who has, is suspected of having, or is at risk for a cancer. Also disclosed herein is a method comprising administering to a subject the plurality of NK cells.

### Natural Killer Cells

Natural killer (NK) cells are a subpopulation of lymphocytes which play a critical role in the innate immune system. NK cells have cytotoxicity against a variety of cells including but not limited to tumor cells and virus-infected cells. In some embodiments, the stem cells described herein are differentiated to Natural Killer cells. In some embodiments, iPSCs are differentiated into NK cells. In some embodiments, the engineered NK cells (such as cells derived from gene-edited iPSCs by differentiation, e.g., iNK cells) have enhanced anti-tumor activity as compared to un-edited or wild type NK cells. In some embodiments, anti-tumor activity of the engineered NK cells is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% relative to control (e.g., un-edited or wild type) NK cells.

In some embodiments, the engineered NK cells exhibit increased cellular lysis capability relative to control cells. In some embodiments, the engineered NK cells of the present disclosure exhibit at least 10% increase in cellular lysis capability (kill at least 10% more target cells), or at least 20% increase in cellular lysis capability (kill at least 20% more target cells), relative to control (e.g., un-edited or wild type) cells. For example, the engineered NK cells of the present disclosure may exhibit an at least at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% increase in cellular lysis capability, relative to control (e.g., un-edited or wild type) cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular lysis capability, relative to control (e.g., un-edited or wild type) cells. In some embodiments, the target cells are T cells. In some embodiments, the target cells are cancer cells. In some embodiments, the target cells are leukemia cells. In some embodiments, the target cells are liver cancer cells. In some embodiments, the target cells are lung cancer cells. In some embodiments, this increase in cellular lysis capability is observed at E:T (effector:targetcell) ratio of at or about 0.1:1. In some embodiments, this increase in cellular lysis capability is observed at E:T (effector: target cell) ratio of at or about 0.5:1. In some embodiments, this increase in cellular lysis capability is observed at E:T (effector:targetcell) ratio of at or about 1:1. In some embodiments, this increase in cellular lysis capability is observed at E:T (effector:targetcell) ratio of at or about 4:1. In some embodiments, this increase in cellular lysis capability is observed at E:T (effector: target cell) ratio of at or about 0.1:1, when the target cell is K562 and when the cells are co-cultured for, e.g., 24 hours. In some embodiments, this increase in cellular lysis capability is observed at E:T (effector:targetcell) ratio of at or about 0.5:1, when the target cell is K562 and when the cells are co-cultured for, e.g., 24 hours. In some embodiments, this increase in cellular lysis capability is observed at E:T (effector:targetcell) ratio of at or about 1:1, when the target cell is K562 and when the cells are co-cultured for, e.g., 24 hours. In some embodiments, this increase in cellular lysis capability is observed at E:T (effector:targetcell) ratio of at or about 0.1:1, when the target cell is RPMI and when the cells are co-cultured for, e.g., 24 hours. In some embodiments, this increase in cellular lysis capability is observed at E:T (effector:targetcell) ratio of at or about 0.5:1, when the target cell is RPMI and when the cells are co-cultured for, e.g., 24 hours. In some embodiments, this increase in cellular lysis capability is observed at E:T (effector:targetcell) ratio of at or about 1:1, when the target cell is RPMI and when the cells are co-cultured for, e.g., 24 hours. In some embodiments, this increase in cellular lysis capability is observed at E:T (effector:targetcell) ratio of at or about 0.1:1, when the target cell is PC-9 and when the cells are co-cultured for, e.g., 24 hours. In some embodiments, this increase in cellular lysis capability is observed at E:T (effector:targetcell) ratio of at or about 0.5:1, when the target cell is PC-9 and when the cells are co-cultured for, e.g., 24 hours. In some embodiments, this increase in cellular lysis capability is observed at E:T (effector:targetcell) ratio of at or about 1:1, when the target cell is PC-9 and when the cells are co-cultured for, e.g., 24 hours. In some embodiments, this increase in cellular lysis capability is observed at E:T (effector:targetcell) ratio of at or about 0.1:1, when the target cell is HepG2 and when the cells are co-cultured for, e.g., 24 hours. In some embodiments, this increase in cellular lysis capability is observed at E:T (effector:targetcell) ratio of at or about 0.5:1, when the target cell is HepG2 and when the cells are co-cultured for, e.g., 24 hours. In some embodiments, this increase in cellular lysis capability is observed at E:T (effector:targetcell) ratio of at or about 1:1, when the target cell is HepG2 and the cells are co-cultured for, e.g., 24 hours.

In some embodiments, the population of cells comprising human NK cells has at least one of the following characteristics, or any combination thereof: (i) an alloimmune T cell reaction of less than 10% (e.g., less than 0.01%, 0.1%, 1%, 2%, 3%, 4%, 5%, 6%, 7%, 8%, 9%, 10%) relative to a population of unmodified human NK cells, (ii) cytotoxic activity resulting in killing more than 50% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of target cells when the population of cells comprising human NK cells are mixed with the target cells at the ratio of 1:1, and (iii) at least 50% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) increase in cellular viability relative to a population of unmodified human NK cells.

In some embodiments, the population of cells comprising human NK cells has at least one of the following characteristics, or any combination thereof: (i) improved persistency, (ii) improved immune evasiveness, (iii) improved cytotoxic activity, (iv) improved antibody-dependent cellular cytotoxicity (ADCC) activity, and (v) improved anti-tumor activity; wherein the characteristics are improved relative to a population of unmodified human NK cells. In some embodiments, the population of cells comprising human NK cells, when co-cultured in vitro with a population of cancer cells, induce cell death of at least 60%, at least 70%, at least 80%, or at least 90% of the population of cancer cells after about 24 hours of co-culture. In some embodiments, the population of cells comprising human NK cells, when co-cultured in vitro with a population of cancer cells, induce cell death of at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of the population of cancer cells after about 24 hours of co-culture.

In some embodiments, the engineered NK cells (and/or e.g., a population of cells comprising the engineered NK cells) express at least one, two, three, four, five, six, seven, eight or all of the following markers: CD45, CD56, CD94, NKG2A, CD16, NKp44, NKp46, KIR2DL4, and KIR3DL2, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells expresses at least one, two, three, four, five or all of the following markers: CD56, NKp44, NKp46, CD94, NKG2A and KIR2DL4, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five, six, seven, eight or all of the following markers: CD45, CD56, CD94, NKG2A, CD16, NKp44, NKp46, KIR2DL4, and KIR3DL2. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five or all of the following markers: CD56, NKp44, NKp46, CD94, NKG2A and KIR2DL4.

In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1.

In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25.

The engineered human NK cells can express at least one, two, three, four or five of the markers of CD56, NKp44, NKp46, CD94, NKG2A, KIR2DL4, and a CAR. The at least one, two, three, four or five markers can be expressed in at least 25%, 30%, 40%, 50%, or 75% of the population of cells (e.g., in 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values of the population of cells). In some embodiments, the CAR is detected by binding to (e.g., staining with) Protein L. Protein L is a peptide isolated from bacteria which can be used to detect light chains of antibodies.

In some embodiments, the engineered NK cells of the present disclosure exhibit an increased cytokine secretion relative to control (e.g., un-edited or wild type) cells. In some embodiments, the engineered NK cells of the present disclosure exhibit about the same cytokine secretion level relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) cytokine secretion level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) cytokine secretion level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) cytokine secretion level relative to control (e.g., un-edited or wild type) cells. The cytokine(s) being measured can be, without limitation any one or more of: TNFα, IFNγ and IL-7. In some embodiments, the level of cytokines (e.g., TNFα, IFNγ and IL-7) secreted by the engineered NK cells is about the same as the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased expression or release of Granzyme B or perforin relative to control (e.g., un-edited or wild type) cells. In some embodiments, the engineered NK cells of the present disclosure exhibit about the same expression or release level of Granzyme B or perforin relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) Granzyme B or perforin expression or release level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) Granzyme B or perforin expression or release level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) Granzyme B or perforin expression or release level relative to control (e.g., un-edited or wild type) cells. In some embodiments, the level of Granzyme B or perforin secreted by the engineered NK cells is about the same as the level in control (e.g., un-edited or wild type) cells, when cells are co-cultured with target cells at the E:T ratio of or about 0.1:1. In some embodiments, the level of Granzyme B or perforin secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of Granzyme B or perforin secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased (e.g., increased by at least 5%, at least 10%, at least 20%, at least 30%, at least 40%, at least 50%, or at least 75%) expression level of CD107a relative to control (e.g., un-edited or wild type) cells. In some embodiments, the engineered NK cells of the present disclosure exhibit about the same expression level of CD107a relative to control (e.g., un-edited or wild type) cells. In some embodiments, engineered NK cells of the present disclosure exhibit a reduced (e.g., reduced by less than 10%, less than 20%, less than 30%, less than 40%, or less than 50%) CD107a expression level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) CD107a expression level relative to control (e.g., un-edited or wild type) cells.

In some embodiments, the engineered NK cells have higher proliferative capacity as compared to un-edited or wild-type NK cells. In some embodiments, the engineered NK cells have approximately the same proliferative capacity compared to un-edited or wild-type NK cells. In some embodiments, the engineered NK cells do not exhibit exhaustion or exhibit a low level of exhaustion (e.g., a level of exhaustion markers associated with a functional NK cell). In some embodiments, exhaustion is detected by detecting a reduced expression of IFNγ, granzyme B, perforin, CD107a, and/or TNFα in cells. In some embodiments, exhaustion is detected by detecting increased expression (e.g., on the surface of the cell) of an exhaustion marker, e.g., PD-1, LAG-3, TIGIT and/or TIM-3. In some embodiments, the engineered NK cells have normal or higher than normal expression of perforin, granzyme B, CD107a, IFNγ and/or TNFα (relative to un-edited or wild-type cells). In some embodiments, the engineered NK cells have lower than normal or no expression of PD-1, LAG-3, TIGIT and/or TIM-3 (relative to un-edited or wild-type cells). In some embodiments, engineered NK cells of the present disclosure exhibit reduced exhaustion, relative to control (e.g., un-edited cells or wild type) NK cells.

In some embodiments, the engineered NK cells of the present disclosure exhibit about the same cellular viability as control (e.g., un-edited or wild-type) cells. In some embodiments, the engineered NK cells of the present disclosure exhibit increased cellular viability relative to control (e.g., un-edited or wild-type) cells. In some embodiments, the engineered NK cells of the present disclosure exhibit at least 10% or at least 20% increase in cellular viability, relative to control cells. For example, the engineered NK cells of the present disclosure may exhibit at least 25%, at least 30%, at least 35%, at least 40%, at least 45%, at least 50%, at least 55%, at least 60%, at least 65%, at least 70%, at least 75%, at least 80%, or at least 90% increase in cellular viability, relative to control cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells. Methods of measuring cell viability are known to those of skill in the art and described herein.

In some embodiments, the engineered NK cells have higher expression of one or more cell cycle genes, one or more cell division genes, and/or one or more DNA replication genes, as compared to un-edited or wild type NK cells. In some embodiments, the engineered NK cells have approximately the same expression of one or more cell cycle genes, one or more cell division genes, and/or one or more DNA replication genes, as compared to un-edited or wild type NK cells.

In some embodiments, gene-edited iPSC cells are differentiated into NK cell having any of the characteristics described herein. In some embodiments, iPSC cells are gene-edited with one or more of the following, B2M null, CIITA null, CISH null, FAS null, CD38 null, FLI1 null, TGFβR1 null, TGFβR2 null, ZEB1 null, PD-1 null, TIGIT null, REGNASE-1 null, NKG2A null, A2a/A2b null, ADAM17 null, SERPIB9 knock-in, HLA-E knock-in, IL15 knock-in, IL15Rα knock-in, GPC3 CAR knock-in, GPR87 CAR knock-in, CD16 knock-in, CD64 knock-in, NKG2D CAR knock-in, BCMA CAR knock-in, CD30 CAR knock-in, CD19-CD20-BCMA CAR knock-in, CD33 CAR knock-in, and/or A33 CAR knock-in, then differentiated into NK cells. In some embodiments, iPSC cells are edited with B2M null, CIITA null, CISH null, FAS null, CD38 null, FLI1 null, TGFβR1 null, TGFβR2 null, SERPIB9 knock-in, HLA-E knock-in, IL15 knock-in, IL15Rα knock-in, GPC3 CAR KI, GPR87 knock-in, CD16 knock-in, CD64 knock-in, and/or NKG2D CAR knock-in, then differentiated into NK cells.

In some embodiments, iPSC cells are gene-edited with one or more of the following, B2M null, CIITA null, CISH null, FAS null, SERPINB9 knock-in, HLA-E knock-in, IL15 knock-in, IL15Rα knock-in (e.g., IL15 and IL15Rα fusion), anti-GPC3 CAR KI, CD64 knock-in, then differentiated into NK cells. In some embodiments, iPSC cells are gene-edited with one or more of the following, B2M null, CIITA null, CISH null, FAS null, SERPIB9 knock-in, HLA-E knock-in, IL15 knock-in, IL15Rα knock-in, anti-GPR87 CAR KI, CD64 knock-in, then differentiated into NK cells. In some embodiments, the iPSCs further comprise TGFβR2 null.

In some embodiments, any of the engineered NK cells described herein have one of more of the following characteristics relative to an un-edited (wild-type) NK cell described herein: increased persistency, increased immune evasiveness, lack of an alloimmune T cell response, increased cytotoxic activity, improved antibody-dependent cellular cytotoxicity (ADCC), or increased anti-tumor activity.

In some embodiments, the population of engineered cells of the present disclosure is engineered (e.g., by use of CRISPR-Cas9 gene-editing) to induce a site-specific disruption in a target gene sequence that eliminates the expression of an allogeneic antigen. In some embodiments, an allogeneic antigen is a major histocompatibility antigen. In some embodiments, a major histocompatibility antigen is a MHC I complex. In some embodiments, the target gene sequence is found in the B2M gene that encodes a protein component of the MHC I complex.

In some embodiments, persistence of the engineered cells is assessed by analyzing their presence and quantity in one or more tissue samples that are collected from a subject following administration of the engineered cells to the subject. In some embodiments, persistence is defined as the longest duration of time from administration to a time wherein a detectable level of the engineered cells is present in a given tissue type (e.g., peripheral blood). In some embodiments, persistence is defined as the continued absence of disease (e.g., complete response or partial response). Determination of the absence of disease and response to treatment are known to those of skill in the art and described herein.

Methods of appropriate tissue collection, preparation, and storage are known to one skilled in the art. In some embodiments, persistence of cells is assessed in one or more tissue samples from a group comprised of peripheral blood, cerebrospinal fluid, tumor, skin, bone, bone marrow, breast, kidney, liver, lung, lymph node, spleen, gastrointestinal tract, tonsils, thymus and prostate. In some embodiments, a quantity of cells is measured in a single type of tissue sample (e.g., peripheral blood). In some embodiments, a quantity of cells is measured in multiple tissue types (e.g., peripheral blood in addition to bone marrow and cerebrospinal fluid). By measuring quantity of cells in multiple tissue types, the distribution of cells throughout different tissues of the body can be determined. In some embodiments, a quantity of cells is measured in one or more tissue samples at a single time point following administration. In some embodiments, a quantity of cells is measured in one or more tissue samples at multiple time points following administration.

A detectable level of the engineered cells in a given tissue can be measured by known methodologies. Methods for assessing the presence or quantity of cells in a tissue of interest are known to those of skill in the art. Such methods include, but are not limited to, reverse transcription polymerase chain reaction (RT-PCR), competitive RT-PCR, real-time RT-PCR, RNase protection assay (RPA), quantitative immunofluorescence (QIF), flow cytometry, northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

As used herein, in some embodiments, persistence is the longest period from the time of administration to a time wherein a detectable level of the engineered cells is measured. In some embodiments, a detectable level of cells is defined in terms of the limit of detection of a method of analysis. The limit of detection can be defined as the lowest quantity of a component or substance that can be reliably and reproducibly measured by an analytical procedure when compared to a tissue sample expected to have no quantity of the component or substance of interest. A non-limiting exemplary method to determine a reproducible limit of detection is to measure the analytical signal for replicates of a zero calibrator relative to a blank sample (Armbruster, D. et al. (2008) Clin Biochem Rev. 29: S49-S52). A blank sample is known to be devoid of an analyte of interest. A zero calibrator is the highest dilution of a test sample of known concentration or quantity that gives analytical signal above that measured for the blank sample. By quantifying the analytical signal for at least 5, 6, 7, 8, 9, 10, 11, 12, 13, 14, 15, 16, 17, 18, 19, 20, 21, 22, 23, 24, 25, 26, 27, 28, 29, or 30 replicates of a zero calibrator, one can determine an average and standard deviation (SD) for the limit of detection of an analytical method of interest. Selection of a method with a suitable limit of detection for quantifying donor T cells in a given tissue can be ascertained by one skilled in the art. In some embodiments, a detectable level of cells is any quantity of cells in a tissue sample that gives an analytical signal above the limit of detection for a method of analysis. In some embodiments, a detectable level of cells is any quantity of cells in a tissue sample that gives an analytical signal that is at least 2 SDs, 3 SDs, 4 SDs, 5 SDs, 6 SDs, 7 SDs, 8 SDs, 9 SDs, or 10 SDs, above the limit of detection for the method of analysis.

It is known that CAR-expressing donor cells can undergo expansion following administration to a recipient. Expansion is a response to antigen recognition and signal activation (Savoldo, B. et al. (2011) J Clin Invest. 121:1822; van der Stegen, S. et al. (2015) Nat Rev Drug Discov. 14:499-509). In some embodiments, following expansion, CAR-expressing engineered cells undergo a contraction period, wherein a portion of the cell population that are short-lived effector cells are eliminated and what remains is a portion of the cell population that are long-lived memory cells. In some embodiments, persistence is a measure of the longevity of the engineered cell population following expansion and contraction. The duration of the expansion, contraction and persistence phases are evaluated using a pharmacokinetic profile. In some embodiments, a pharmacokinetic (PK) profile is a description of the cells measured in a given tissue over time and is readily ascertained by one skilled in the art by measuring the cells in a given tissue (e.g., peripheral blood) at multiple time points. In some embodiments, a measure of a PK profile provides a method of evaluating or monitoring the effectiveness of the engineered cell therapy in a subject (e.g., having cancer). In some embodiments, a measure of a PK profile provides a method of evaluating the persistence of the engineered cells in a subject. In some embodiments, a PK profile provides a method of evaluating the expansion of the engineered cells in a subject. In some embodiments, a measure of persistence of engineered cells in a subject is used to evaluate the effectiveness of engineered cell therapy in a subject. In some embodiments, a measure of expansion of engineered cells in a subject is used to evaluate the effectiveness of engineered cell therapy in a subject.

A PK profile can be prepared by measuring a quantity of engineered cells in a sample of a given tissue type (e.g., peripheral blood) collected from a recipient and repeating the assessment at different time points. In some embodiments, a baseline tissue sample is collected from a recipient no more than 1 day, 2 days, 3 days, 4 days, 5 days, 6 days, 7 days, 8 days, 9 days, 10 days, 12 days, 13 days, 14 days, or 15 days prior to administration. In some embodiments, tissue collection from a recipient is performed within 0.25-2 hours, within 1-3 hours, within 2-6 hours, within 3-11 hours, within 4-20 hours, or within 5-8 hours of the time of administration of engineered cells. In some embodiments, tissue collection from a recipient is performed on a daily basis starting on day 1, day 2, day 3, or day 4 and continuing through at least day 5, day 6, day 7, day 8, day 9, day 10, day 11, day 12, day 13, day 14, day 15, day 16, day 17, day 18, day 19, or day 20. In some embodiments, tissue collection from a recipient is performed at least 1 time, 2 times, 3 times, 4 times, 5 times, or 6 times per week for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks following administration of cells. In some embodiments, tissue collection from a recipient is performed at least 1 time, 2 times, 3 times, 4 times, 5 times, or 6 times per month for up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months following administration of cells. In some embodiments, tissue collection from a recipient is performed at least 1 time, 2 times, 3 times, 4 times, 5 times, or 6 times per year for up to 1 year, 2 years, 3 years, 4 years, 5 years, 6 year, 7 years, 8 years, 9 years, or 10 years following administration of cells.

In some embodiments, engineered cell persistence is determined as the duration of time from administration wherein a quantity of engineered cells is present that is at least 0.005-0.05%, 0.01-0.1%, 0.05-0.5%, 0.1-1%, 0.5%-5%, 1-10%, 5%-10%, or 10%-15% (e.g., at least 1%, 5%, 10%, or 15%) of the peak quantity of engineered cells. In some embodiments, a persistence of cells is determined by comparing the quantity of cells measured in a given tissue type (e.g., peripheral blood) to the peak quantity of cells that is measured in the same tissue type. In some embodiments, a persistence of cells is determined by comparing the quantity of cells measured in a given subject (e.g., peripheral blood) to the peak quantity of cells that is measured in the same subject. In some embodiments, a persistence of cells is determined by comparing the quantity of cells measured in a given subject (e.g., peripheral blood) to the peak quantity of cells that is measured in a different subject (i.e., a subject with partial response, a subject with complete response).

In some embodiments, a persistence of engineered cells is present in one or more tissue types (e.g., peripheral blood) following administration wherein engineered cells are administered on day 1. In some embodiments, a persistence of engineered cells is present in one or more tissue types (e.g., peripheral blood) up to 1 day, 2 days, 3 days, 4, days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, or 35 days following administration wherein engineered cells are administered on day 1. In some embodiments, a persistence of engineered cells is present in one or more tissue types (e.g., peripheral blood) up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 21 months, 22 months, 23 months, or 24 months following administration of engineered cells). In some embodiments, a persistence of engineered cells is measured in one or more tissue types (e.g., peripheral blood) up to 1 year, 2 years, 3 years, 4 years, 5 years, 6 years, 7 years, 8 years, 9 years, and 10 years following administration of engineered cells. In some embodiments, a persistence of engineered cells that is at least 10-25 days, at least 25-50 days, at least 50-100 days, at least 100-364 days, at least one year, at least two years, at least three years, at least four years or at least five years from administration wherein engineered cells are administered on day 1 is indicative of a response in a recipient (e.g., complete response or partial response).

### Isolation and Purification of Cells

### Purification

In some embodiments, the population of gene-edited cells (e.g., iPSC, iNK, or NK cells) described herein are activated and/or expanded before or after genome editing. In some embodiments, iPSCs are differentiated after gene-editing. In some embodiments, cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to genome editing.

Disclosed herein includes a method for substantially isolating cells that express a detectable level of a surface protein (e.g., B2M) from a population of cells comprising engineered NK cells comprising a combination of any of the gene edits described herein.

In some embodiments, the disclosure provides a method for isolating a population of cells engineered to express one or CAR comprising: providing the population of cells wherein the engineered CAR cells comprise a disrupted CIITA gene, a disrupted B2M gene, a disrupted ADAM17 gene, a disrupted FAS gene, a disrupted CISH gene, a disrupted CD38 gene, a disrupted FLI1 gene, a disrupted TGFβR1 gene, a disrupted TGFβR2 gene, and/or a disrupted REGNASE-1 gene; and isolating the population of cells expressing the one or more CAR (e.g. such that >99% of the population comprises the CAR expressing cells).

In some embodiments, the disclosure provides a population of cells comprising engineered CAR NK cells described herein, wherein less than 0.5% of the cells in the population express a detectable level of B2M, CIITA, FAS, CISH, CD38, FLI1, TGFBR1, TGFBR2, ADAM17, and/or REGNASE-1. In some embodiments, the disclosure provides a population of cells comprising engineered CAR NK cells described herein, wherein less than 0.1%, less than 0.2%, less than 0.3%, less than 0.4%, less than 0.5%, less than 1%, less than 2%, less than 3%, less than 4%, less than 5% or less than 10% of the cells in the population express a detectable level of B2M, CIITA, FAS, CISH, CD38, FLI1, TGFβR1, TGFβR2, ADAM17, and/or REGNASE-1.

Removal of a subset of cells from a population can be performed using conventional cell purification methods. Non-limiting examples of cell sorting methods include fluorescence-activated cell sorting, immunomagnetic separation, chromatography, and microfluidic cell sorting. In some embodiments, CAR-expressing cells are removed from a population of cells comprising engineered NK cells by immunomagnetic separation. In some embodiments, HLA-E-expressing cells are removed from a population of cells comprising engineered NK cells by immunomagnetic separation.

In some embodiments, genome edited cells are sorted into single cells. In some embodiments, single cell isolates of gene-edited cells are grown into single cell clonal populations. In some embodiments, multiple single-cell clones are generated. In some embodiments, an edited clone is expanded to generate a master cell bank (MCB).

In some embodiments, the population of gene-edited cells (e.g., iPSC, iNK, or NK cells) described herein are activated and/or expanded before or after genome editing. In some embodiments, iPSCs are differentiated after gene-editing. In some embodiments, cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to genome editing.

Provided herein includes a method for substantially isolating cells that express a detectable level of a surface protein (e.g., B2M) from a population of cells comprising engineered NK cells comprising a combination of any of the gene edits described herein.

In some embodiments, the disclosure provides a method for isolating a population of cells engineered to express one or CAR comprising: providing the population of cells wherein the engineered CAR cells comprise a disrupted CIITA gene, a disrupted B2M gene, a disrupted ADAM17 gene, a disrupted FAS gene, a disrupted CISH gene, a disrupted CD38 gene, a disrupted FLI1 gene, a disrupted TGFBR1 gene, a disrupted TGFBR2 gene, and/or a disrupted REGNASE-1 gene; and isolating the population of cells expressing the one or more CAR (e.g. such that >99% of the population comprises the CAR expressing cells).

In some embodiments, the disclosure provides a population of cells comprising engineered CAR NK cells described herein, wherein less than 0.5% of the cells in the population express a detectable level of B2M, CIITA, FAS, CISH, CD38, FLI1, TGFBR1, TGFBR2, ADAM17, and/or REGNASE-1. In some embodiments, the disclosure provides a population of cells comprising engineered CAR NK cells described herein, wherein less than 0.1%, less than 0.2%, less than 0.3%, less than 0.4%, less than 0.5%, less than 1%, less than 2%, less than 3%, less than 4%, less than 5% or less than 10% of the cells in the population express a detectable level of B2M, CIITA, FAS, CISH, CD38, FLI1, TGFβR1, TGFβR2, ADAM17, and/or REGNASE-1.

Removal of a subset of cells from a population can be performed using conventional cell purification methods. Non-limiting examples of cell sorting methods include fluorescence-activated cell sorting, immunomagnetic separation, chromatography, and microfluidic cell sorting. In some embodiments, CAR-expressing cells are removed from a population of cells comprising engineered NK cells by immunomagnetic separation. In some embodiments, HLA-E-expressing cells are removed from a population of cells comprising engineered NK cells by immunomagnetic separation.

In some embodiments, genome edited cells are sorted into single cells. In some embodiments, single cell isolates of gene-edited cells are grown into single cell clonal populations. In some embodiments, multiple single-cell clones are generated. In some embodiments, an edited clone is expanded to generate a master cell bank (MCB).

### Formulations and Administrations

### Formulation and Delivery for Gene Editing

Guide RNAs, polynucleotides, e.g., polynucleotides that encode any protein described herein or polynucleotides that encode an endonuclease, and endonucleases as described herein may be formulated and delivered to cells in any manner known in the art.

Guide RNAs and/or polynucleotides may be formulated with pharmaceutically acceptable excipients such as carriers, solvents, stabilizers, adjuvants, diluents, etc., depending upon the particular mode of administration and dosage form. Guide RNAs and/or polynucleotides compositions can be formulated to achieve a physiologically compatible pH, and range from a pH of about 3 to a pH of about 11, about pH 3 to about pH 7, depending on the formulation and route of administration. In some cases, the pH can be adjusted to a range from about pH 5.0 to about pH 8. In some cases, the compositions can comprise a therapeutically effective amount of at least one compound as described herein, together with one or more pharmaceutically acceptable excipients. Optionally, the compositions can comprise a combination of the compounds described herein, or can include a second active ingredient useful in the treatment or prevention of bacterial growth (for example and without limitation, anti-bacterial or anti-microbial agents), or can include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 2011, 18:1127-1133 (which focuses on non-viral delivery vehicles for siRNA that are also useful for delivery of other polynucleotides).

For polynucleotides of the disclosure, the formulation may be selected from any of those taught, for example, in International Application PCT/US2012/069610. Polynucleotides, such as guide RNA, sgRNA, and mRNA encoding an endonuclease, may be delivered to a cell or a subject by a lipid nanoparticle (LNP). An LNP can refer to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. Limitations of cationic lipids include low efficacy owing to poor stability and rapid clearance, as well as the generation of inflammatory or anti-inflammatory responses. LNPs may also be comprised of hydrophobic lipids, hydrophilic lipids, or both hydrophobic and hydrophilic lipids.

Any lipid or combination of lipids known in the art can be used to produce an LNP. Examples of lipids used to produce LNPs include, but are not limited to, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20. The lipids can be combined in any number of molar ratios to produce an LNP. In addition, the polynucleotide(s) can be combined with lipid(s) in a wide range of molar ratios to produce an LNP.

A recombinant adeno-associated virus (rAAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): an rAAV genome, AAV rep and cap genes separate from (i.e., not in) the rAAV genome, and helper virus functions. The AAV rep and cap genes may be from any AAV serotype for which recombinant virus can be derived, and may be from a different AAV serotype than the rAAV genome ITRs, including, but not limited to, AAV serotypes described herein. Production of pseudotyped rAAV is disclosed in, for example, international patent application publication number WO 01/83692.

### Formulation and Administration of Cells

Genetically modified cells, as described herein may be formulated and administered to a subject by any manner known in the art. Disclosed herein include compositions. In some embodiments, the compositions comprise any population of cells described herein. Also, disclosed herein include methods of obtaining cells for administration to a subject in need thereof, the method comprising: (a) obtaining or having obtained any of the population of cells described herein, and (b) maintaining the population of cells for a time and under conditions sufficient for the one or more engineered cells to differentiate into lineage-restricted progenitor cells or fully differentiated somatic cells. There are provided, methods for treating a subject in need thereof. In some embodiments, the method comprises: (a) obtaining or having obtained any of the population of cells disclosed herein; and (b) administering the lineage-restricted progenitor cells or fully differentiated somatic cells to the subject.

The terms “administering,” “introducing, “implanting,” “engrafting” and “transplanting” are used interchangeably in the context of the placement of cells, e.g., progenitor cells, into a subject, by a method or route that results in at least partial localization of the introduced cells at a desired site. The cells e.g., progenitor cells, or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment. In some embodiments, a genetically modified cell as described herein is viable after administration to a subject for a period that is longer than that of an unmodified cell.

In some embodiments, a composition comprising cells as described herein are administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein. Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

In some embodiments, a composition comprising cells may be administered to a subject, e.g., a human subject, who has, is suspected of having, or is at risk for a disease. In some embodiments, a composition may be administered to a subject who does not have, is not suspected of having or is not at risk for a disease. In some embodiments, a subject is a healthy human. In some embodiments, a subject e.g., a human subject, who has, is suspected of having, or is at risk for a genetically inheritable disease. In some embodiments, the subject is suffering or is at risk of developing symptoms indicative of a disease. In some embodiments, the subject is suffering from, suspected of having, or is at risk for cancer.

### Treatment Methods

The methods, compositions and kits disclosed herein can be used to treat various types of cancer, including but not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (e.g., prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

Provided herein, in some embodiments, are methods for treating cancer (e.g., leukemias, e.g., acute myeloid leukemia) using any engineered cells described herein (or any population of cells described herein). Non-limiting examples of leukemias that may be treated as provided herein include chronic lymphocytic leukemia (CLL), multiple myeloma (MM), Hodgkin's lymphoma, non-Hodgkin lymphomas (e.g., diffuse large B-cell lymphoma (DLBCL), high grade B-cell lymphoma, transformed follicular lymphoma (FL), grade 3B FL, chronic lymphocytic leukemia (C-CLL), Richter's transformation of CLL, T cell lymphoma, T cell leukemia, acute myeloid leukemia (AML), and acute lymphoblastic leukemia (ALL). Solid cancers that may be treated include, without limit, lung cancer, clear cell renal cell carcinoma (ccRCC), thyroid cancer, nasopharyngeal cancer, non-small cell lung (NSCLC) cancer, pancreatic cancer, melanoma, ovarian cancer, glioblastoma, liver cancer, colon cancer, or cervical cancer. In some embodiments, provided herein is a method of treating cancer in a subject (e.g., human) in need thereof, comprising administering any engineered cell described herein to the subject (e.g., wherein the subject has or has been diagnosed with cancer). In some embodiments, provided herein is a method of treating a non-Hodgkin lymphoma (e.g., diffuse large B-cell lymphoma (DLBCL), high grade B-cell lymphoma, transformed follicular lymphoma (FL), grade 3B FL, and Richter's transformation of CLL in a subject (e.g., human) in need thereof, comprising administering any engineered cell described herein to the subject (e.g., wherein the subject has or has been diagnosed with a non-Hodgkin lymphoma, or is at risk of a non-Hodgkin lymphoma). In some embodiments, the subject (e.g., a human) has (e.g., has been diagnosed with) a relapsed and/or refractory non-Hodgkin lymphoma. In some embodiments, the subject (e.g., a human) has (e.g., has been diagnosed with) a non-relapsed or early stage non-Hodgkin lymphoma. In some embodiments, provided herein is a method of treating chronic lymphocytic leukemia (CLL) or acute lymphoblastic leukemia (ALL) in a subject (e.g., human) in need thereof, comprising administering any engineered cell described herein to the subject (e.g., wherein the subject has or has been diagnosed with CLL or ALL). In some embodiments, the subject (e.g., a human) has (e.g., has been diagnosed with) a relapsed and/or refractory CLL or ALL. In some embodiments, the subject (e.g., a human) has (e.g., has been diagnosed with) a non-relapsed or early stage CLL or ALL. The engineered cell can be administered at any dose described herein, in particular, in a therapeutically effective amount. In some embodiments, a human being treated is an adult, e.g., a human over 18 years of age. In some embodiments, a human being treated is under 18 years of age.

Some embodiments provide methods for the treatment of cancer comprising administering to a subject in need thereof engineered cells of the disclosure. In some embodiments, the methods comprise delivering the engineered cells (e.g., anti-GPC3 CAR NK cells) of the present disclosure to a subject having a cancer (e.g., leukemia, liver cancer, or lung cancer), wherein cancer cells express GPC3. In some embodiments, the methods comprise delivering the engineered cells (e.g., anti-CD30 CAR NK cells) of the present disclosure to a subject having a cancer (e.g., leukemia), wherein cancer cells express CD30. In some embodiments where the disease being treated is a non-Hodgkin lymphoma, and the cells used express a CD30 CAR (e.g., anti-CD30 CAR NK cells). In some embodiments the disease being treated is liver cancer or lung cancer, and the cells used express a GPC3 CAR (e.g., anti-GPC3 CAR NK cells). In some embodiments the disease being treated is liver cancer or lung cancer, and the cells used express a GPR87 CAR (e.g., anti-GPR87 CAR NK cells).

The step of administering can include the placement (e.g., transplantation) of cells, e.g., engineered NK cells, into a subject, by a method or route that results in at least partial localization of the introduced cells at a desired site, such as tumor, such that a desired effect(s) is produced. Engineered cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment. For example, in some embodiments, an effective amount of engineered NK cell is administered via a systemic route of administration, such as an intraperitoneal or intravenous route.

A subject may be any subject for whom diagnosis, treatment, or therapy is desired. In some embodiments, the subject is a mammal. In some embodiments, the subject is a human. In some embodiments, an engineered NK cell population being administered according to the methods described herein comprises gene edited hematopoietic cells (e.g., NK cells) differentiated from gene-edited stem cells (e.g., iPSC cells). In some embodiments, an engineered cell population (e.g., comprising NK cells) being administered according to the methods described herein does not induce toxicity in the subject, e.g., the engineered NK cells do not induce toxicity in non-cancer cells. In some embodiments, an engineered cell population (e.g., NK cells) being administered does not trigger complement mediated lysis, or does not stimulate antibody-dependent cell mediated cytotoxicity (ADCC). In some embodiments, the subject being treated has no chronic immune suppression.

An effective amount refers to the amount of a population of engineered cells (e.g., NK cells) needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, a subject is administered a population of cells comprising any of the engineered NK cells disclosed herein. In some embodiments, a subject is administered a population of cells comprising any of the engineered NK cells disclosed herein at a dose in the range of about 1×10⁷ to 3×10⁸ engineered cells. In some embodiments, a subject is administered a population of cells comprising any of the engineered cells disclosed herein at a dose in the range of about 3×10⁷ to 3×10⁸ engineered cells. In some embodiments, the cells are derived from iPSCs. In some embodiments, the cells are expanded in culture prior to administration to a subject in need thereof.

Modes of administration include but are not limited to injection and infusion. In some embodiments, injection includes, without limitation, intravenous, intrathecal, intraperitoneal, intraspinal, intracerebrospinal, and intrasternal infusion. In some embodiments, the route is intravenous. In some embodiments, cells are administered as a bolus or by continuous infusion (e.g., intravenous infusion) over a period of time. In some embodiments, cells described herein are administered in several doses over a period of time (e.g., several infusions over a period of time). The cells described herein can be administered in a single dose or in 2, 3, 4, 5, 6 or more doses (or infusions). In some embodiments, the subject being treated is dosed (e.g., with an infusion) about every 1, 2, 3, 4, 5, 6, 7 or 8 weeks. In some embodiments, the subject being treated is dosed (e.g., with an infusion) every 2-4 weeks (e.g., every 2 weeks, 3 weeks or 4 weeks). In some embodiments, engineered cells (e.g., NK cells) are administered systemically, which refers to the administration of a population of cells other than directly into a target site, tissue, or organ, such that it enters, instead, the subject's circulatory system and, thus, is subject to metabolism and other like processes.

The efficacy of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

Disclosed herein include methods of treating cancer. In some embodiments, the method comprises administrating NK cells obtained by the methods disclosed herein (e.g., NK cells derived from cells engineered to express an anti-GPR87 CAR) to a subject with cancer, thereby inhibiting progression of the cancer. In some embodiments, the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, pancreatic cancer, liver cancer, melanoma, ovarian cancer, glioblastoma, or cervical cancer. In some embodiments, the liver cancer is hepatocellular carcinoma. In some embodiments, the lung cancer is non-small cell lung cancer.

The dosage of the NK cells of the disclosure administered to a subject (e.g., a subject suffering from cancer) can vary. The method can comprise administering at most, at least, or about 1×10² to 1×10¹⁰ per gram (e.g., per gram of body weight) to the subject (e.g., 100, 1000, 1×10⁴, 1×10⁵, 1×10⁶, 1×10⁷, 1×10⁸, 1×10⁹, 1×10¹⁰ cells per gram or a number or a range between any two of these values). The method can comprise administering about 1×10⁶ NK cells per gram to the subject.

The NK cells can be administered to the subject once. The NK cells can be administered to the subject more than once. The NK cells can be administered to the subject at least three times. The NK cells can be administered to the subject in a cycle of at least 7 days. The NK cells can be administered to the subject one, two, or three times in a week. In some embodiments, the NK cells are administered to the subject three times in seven to eight days.

The treatment of the present disclosure can comprise administration of NK cells obtained by the disclosed method (e.g., that are engineered to express anti-GPR87 CAR) for a desired duration in a cycle. The administration of NK cells can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be, for example, once every two days, every three days, every four days, every five days, every six days, or every seven days. The length of the desired duration can vary, for example, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the NK cells for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of NK cells for, or for at least, four days, five days, six days, seven days, eight days, nine days, ten days, or a range between any two of these values, in a cycle (e.g., a cycle of at least 21 days (e.g., 21 to 28 days)). The administration of NK cells in a single cycle of the treatment can be continuous or with one or more intervals (e.g., one day or two days of break). In some embodiments, the treatment comprises administration of the NK cells for three days in a cycle of 7 to 8 days. Depending on the needs of inhibition/reversion of cancer progression in the subject, the subject can receive one, two, three, four, five, six, or more cycles of treatment.

Inhibiting progression of the cancer can comprise inhibition of growth of one or more tumors in the subject and/or reducing the number of cancer cells detected in the subject; relative to an untreated subject. Inhibiting progression of the cancer can comprise inhibition of growth of one or more tumors in the subject and/or reducing the number of cancer cells detected in the subject; relative to the subject prior to administration of the NK cells. In some embodiments, the number of cancer cells detected in the subject increases by no more than 0.5-fold after administration of the NK cells, following one or more cycles of treatment. In some embodiments, the number of cancer cells detected in the subject does not significantly increase following one or more cycles of treatment. In some embodiments, the number of cancer cells detected in the subject decreases following one or more cycles of treatment. In some embodiments, the growth of at least one of the one or more tumors in the subject is inhibited by at least about 70%, about 75%, about 80%, about 85%, about 90%, about 95%, or about 100%, following one or more cycles of treatment. In some embodiments, the subject is tumor-free following one or more cycles of treatment. Methods for determining effectiveness of treatment (e.g., inhibition of the progression of the cancer) are known by persons skilled in the art, and are also described below.

### Methods for Determining Treatment Efficacy and Status for Cancer

Also disclosed herein include methods for determining clinical outcome for a treatment of cancer of the present disclosure, monitoring of the treatment, determining responsiveness of a subject to the treatment, determining the status of the cancer in a subject, and improving treatment outcome. The methods can be used to guide the treatment, provide treatment recommendations, and/or reduce or avoid unnecessary ineffective treatment for patients.

In some embodiments, determining clinical outcome for a treatment of cancer of the present disclosure, monitoring of the treatment, determining responsiveness of a subject to the treatment, and/or determining the status of the cancer in a subject comprises determining approximate cancer cell number, and/or measurement of tumor size and/or inhibition of tumor growth. Inhibiting progression of the cancer can comprise inhibition of growth of one or more tumors in the subject. Inhibiting progression of the cancer can comprise reducing the number of cancer cells detected in the subject. The term “inhibition of tumor growth” can refer to causing a reduction in or complete cessation of tumor growth and/or causing a regression in tumor size (e.g., volume). The term “tumor volume” or “tumor size” can refer to the total size of the tumor, which can include the tumor itself plus affected lymph nodes if applicable. Tumor size can be determined by a variety of methods known in the art, such as, e.g. by measuring the dimensions of the tumor using calipers, computed tomography (CT) or magnetic resonance imaging (MRI) scans, mammography, and X-ray; and calculating the volume using equations based on, for example, the z-axis diameter, or on standard shapes such as the sphere, ellipsoid, or cube. Tumor size may be assessed at any time before, during or following at least one cycle of treatment with engineered NK cells. Tumor size can be assessed at a first timepoint, and at one or more additional timepoints. In some embodiments, tumor size can be assessed in the subject and, e.g., an untreated subject at equivalent timepoints (e.g., at a first timepoint, and at one or more additional timepoints). Tumor growth can be determined by, e.g., measuring tumor size at a first timepoint and measuring tumor size at one or more additional timepoints. In some embodiments, increased inhibition of tumor growth in the subject indicates the subject as responsive to the cancer treatment.

The inhibition of growth of at least one of the one or more tumors in the subject can be increased by, by about, by at least, or by at least about 25%, 30%, 35%, 40%, 50%, 55%, 60%, 65%, 70%, 75%, 80%, 85%, 90%, 95%, 100% or a number or a range between any two of these values, relative to an untreated subject or the subject prior to administration of the NK cells, following one or more cycles of treatment. The growth of at least one of the one or more tumors in the subject can be inhibited by, by about, by at least, or by at least about 50%, 55%, 60%, 65%, 70%, 75%, 80%, 85%, 90%, 95%, 100% or a number or a range between any two of these values relative to an untreated subject or the subject prior to administration of the NK cells, following one or more cycles of treatment. The growth of at least one of the one or more tumors in the subject can be inhibited by, by about, by at least, or by at least about 70%, 75%, 80%, 85%, 90%, 95%, 100% or a number or a range between any two of these values relative to an untreated subject or the subject prior to administration of the NK cells, following one or more cycles of treatment. The subject can be tumor-free following one or more cycles of treatment.

In some embodiments, the number of cancer cells detected in the subject increases by no more than 0.5-fold (e.g., increases by 0.5-fold, 0.25-fold, 0.1-fold or less) after administration of the NK cells, following one or more cycles of treatment. In some embodiments, the number of cancer cells detected in the subject decreases by 50% or more in the subject (e.g., by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values), relative to an untreated subject or to the subject prior to administration of the NK cells. In some embodiments, the number of cancer cells detected in the subject does not significantly increase following one or more cycles of treatment. In some embodiments, the number of cancer cells detected in the subject decreases following one or more cycles of treatment.

In some embodiments, the method comprises measuring expression of one or more markers in the subject (e.g., in a sample obtained from the subject). The method can comprise: measuring expression of one or more markers for apoptosis, DNA damage, cell cycle or any combination thereof in the subject before and/or after the subject is administered the NK cells. The one or more markers can comprise cleaved-caspase3, γ-H2AX, phosphorylated CHK1, phosphorylated CHK2, or any combination thereof.

A method of determining responsiveness of a subject to a treatment comprising NK cells of the disclosure (e.g., that are engineered to express an anti-GPC3 or an anti-GPR87 CAR) can comprise, for example, analyzing a sample (e.g., a biopsy sample) obtained from a subject with cancer, wherein the subject is undergoing a treatment and/or has received the treatment, thereby determining the responsiveness of the subject to the treatment. Samples can be obtained by any method known in the art. In some embodiments, the biological sample is obtained from an animal subject, such as a human subject. A biological sample is any solid or fluid sample obtained from, excreted by or secreted by any living organism, (including samples from a healthy or apparently healthy human subject or a human patient affected by cancer). A biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be obtained from any organ or tissue (including a biopsy, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). The sample can also include circulating tumor cells (which can be identified by cell surface markers). In some embodiments, samples are used directly (e.g., fresh or frozen), or can be manipulated prior to use, for example, by fixation (e.g., using formalin) and/or embedding in wax (such as formalin-fixed paraffin-embedded (FFPE) tissue samples). It can be appreciated that any method of obtaining tissue from a subject can be utilized, and that the selection of the method used will depend upon various factors such as the type of tissue, age of the subject, or procedures available to the practitioner.

The levels of the one or more markers in the subject (e.g., in a biopsy sample obtained from the subject) can be measured by any method known in the art. In some embodiments, the one or more markers are detected by immunofluorescence, mass cytometry (CyTOF), FACS, drop-seq, RNA-seq, single cell qPCR, MERFISH (multiplex (in situ) RNA FISH), microarray and/or by in situ hybridization. Other methods including absorbance assays and colorimetric assays are known in the art and may be used herein. In some aspects, measuring expression of the one or more markers comprises measuring protein expression levels. Protein expression levels may be measured, for example, by performing a Western blot, an ELISA, immunohistochemistry or binding to an antibody array. In another aspect, measuring expression of the one or more markers comprises measuring RNA expression levels. RNA expression levels may be measured by performing RT-PCR, Northern blot, an array hybridization, or RNA sequencing methods.

An enzyme-linked immunosorbent assay, or ELISA, may be used to measure the differential expression of a plurality of markers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

Detection of the one or more markers can be by FACS. The term “fluorescent activated cell sorting” or “FACS”, as used herein, refers to a technique for counting, examining, and sorting microscopic particles suspended in a stream of fluid. It allows simultaneous multiparametric analysis of the physical and/or chemical characteristics of single cells flowing through an optical and/or electronic detection apparatus. Generally, a beam of light (usually laser light) of a single wavelength is directed onto a hydro-dynamically focused stream of fluid. A number of detectors are aimed at the point where the stream passes through the light beam; one in line with the light beam (Forward Scatter, correlates to cell volume) and several perpendicular to the beam, (Side Scatter, correlates to the inner complexity of the particle and/or surface roughness) and one or more fluorescent detectors. Each suspended particle passing through the beam scatters the light in some way, and fluorescent chemicals found in the particle or attached to the particle may be excited into emitting light at a lower frequency than the light source. By analyzing the combinations of scattered and fluorescent light picked up by the detectors it is then possible to derive information about the physical and chemical structure of each individual particle.

Detection of the one or more markers may involve a cell sorting step to enrich for cells of interest and thus facilitate or enhance their sensitive and specific detection. Cell sorting techniques are commonly based on tagging the cell with antibody against the cell membrane antigen specific to the target subpopulation of cells. The antibody is conjugated to a magnetic bead and/or fluorophore or other label to enable cell sorting and detection. Such methods may include affinity chromatography, particle magnetic separation, centrifugation, or filtration, and flow cytometry (including fluorescence activated cell sorting; FACS).

RNA can be isolated from the cancer cells and can be sequenced by any method known in the art for determining expression of one or more markers. Methods of preparing cDNA are known in the art. Single cells may be sequenced for detection of at least one of the one or more markers. Single cells can be, for example, divided into single droplets using a microfluidic device. The single cells in such droplets may be further labeled with a barcode.

A method of determining responsiveness of a subject to a treatment comprising engineered NK cells of the disclosure can comprise, for example, analyzing circulating tumor DNA (ctDNA) of a subject with cancer, the subject is undergoing a treatment and/or has received the treatment, thereby determining the responsiveness of the subject to the treatment. For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

### Persistence of the Engineered Cells In Vivo

The engineered cells of the disclosure comprise one or more deletions and/or insertions that can, in some embodiments, advantageously contribute to the persistence (e.g., survival) of the engineered cells in a subject. The NK cells can persist in the subject for at least one week following administration.

In some embodiments, the number of NK cells detected in the subject decreases by less than 25% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or a number or a range between any two of these values) one week after administration. In some embodiments, the number of NK cells detected in the subject decreases by less than 50% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or a number or a range between any two of these values) two weeks after administration. In some embodiments, the NK cells are localized to the site of the cancer following administration.

### Lymphodepletion Conditioning Therapy

In some embodiments, any engineered cells described herein (or any population of cells described herein) are administered to a subject (e.g., a human patient having a cancer, e.g., a non-Hodgkin lymphoma) after a subject has received a lymphodepleting regimen.

In some embodiments, the lymphodepleting regimen comprises administering at least one chemotherapeutic agent (e.g., cyclophosphamide). In some embodiments, the lymphodepleting regimen comprises administering at least two chemotherapeutic agents (e.g., cyclophosphamide and fludarabine). In some embodiments, the first dose (e.g., infusion) of the engineered cells described herein is administered to a subject after lymphodepletion.

### EXAMPLES

Some aspects of the embodiments discussed above are disclosed in further detail in the following examples, which are not in any way intended to limit the scope of the present disclosure.

### Example 1

### Cell Maintenance and Expansion

Maintenance of hiPSCs. Cells of human induced pluripotent stem cell (hiPSC) line were maintained in STEMFLEX™ Complete media (Life Technologies, A3349401) with single cell passaging using ACCUTASE® (Stemcell Technologies 07920 or equivalent) on BIOLAMININ 521 LN (LN521), BIOLAMININ 511 LN (LN511), or Recombinant Laminin iMatrix-511 E8 (AMSBIO, AMS.892 011). For passaging, 1% REVITACELL™ Supplement (100×) was added.

Single cell cloning of hPSCs. For single cell cloning, hiPSCs were fed with STEMFLEX™ Complete media (Life Technologies, A3349401) with 1% REVITACELL™ Supplement (100×) (ThermoFisher Cat #A2644501). Following dissociation with ACCUTASER, the cells were sorted as a single cell per well of a pre-coated plate. The 96 well plates were pre-coated with a 1:10 or a 1:20 dilution of BIOLAMININ 521 LN (LN521) in DPBS, calcium, magnesium (Life Technologies, 14040133) for 2 hours at 37° C. The WOLF FACS-sorter (Nanocellect) was used to sort single cells into the wells. The plates were pre-filled with 100-200 μL of STEMFLEX™ Complete with REVITACELL™ Supplement (100×) and 4 μL/mL of Recombinant Laminin iMatrix-511 E8 (AMSBIO, AMS.892 011). Three days post cell seeding, the cells were fed with fresh STEMFLEX™ and continued to be fed every other day with 100-200 μL of media. After 10 days of growth, the cells were fed daily with STEMFLEX™ until day 12-16. At this time, the plates were dissociated with ACCUTASE® and the collected cell suspensions were split 1:2 with half going into a new 96 well plate for maintenance and half going into a DNA extraction solution QuickExtract™ DNA Extraction Solution (Lucigen). Following DNA extraction, PCR was performed to assess presence or absence of desired gene edits at the targeted DNA locus. Sanger sequencing was used to verify desired knock-out (KO) edits.

Expansion of single cell derived hiPSCs clones. Successfully targeted clones were passaged from 96-well plates to 24-well plates using STEMFLEX™ and BIOLAMININ 521 or Recombinant Laminin iMatrix-511 E8 and 1% REVITACELL™ Supplement (100×). Following expansion in 24-well plates, the cells were passaged onto 6-well plates and then T25 and larger flask formats.

### Example 2

Differentiating Stem Cells into Natural Killer Cells-Protocol 1

Protocol 1 was utilized to differentiate stem cells, such as wild-type and/or edited induced pluripotent stem (iPS) cells, into hematopoietic stem and progenitor cells (HSPCs) and then into natural killer (NK) cells. Prior to differentiation, frozen iPS cells were thawed and re-suspended in NK-MED-001 medium (Table 1). Flasks pre-coated with laminin-521 were used for cell culturing. Medium was changed daily using NK-MED-002 (Table 2) medium until cells were used for differentiation.

NK Cell Differentiation. iPS cells were differentiated using the following steps:
Day −1 (afternoon), iPSC aggregation: NK-MED-002 (Table 2) medium was aspirated from flasks containing iPSC and the cells were washed with DPBS (no calcium, no magnesium) (Thermo Fisher Scientific, 14190250). DPBS was aspirated and 2 mL ACCUTASE® (Innovative Cell Technologies, AT-104) was added per T25 flask (or 80 μL of ACCUTASE® per 1 cm²). Cells were incubated at 37° C. for 3-5 min or until all the colonies detached. Accutase digested cells were diluted with NK-MED-002 medium to a ratio of at least 3:1 (NK-MED-002: ACCUTASE®). Cells were gently resuspended and transferred to a conical tube. Enough NK-MED-002 medium was added to cells to dilute the ACCUTASE® to a ratio of 4:1 (NK-MED-002: ACCUTASE®). Cells were pelleted and re-suspended in 10 ml of NK-MED-003 medium (Table 3). Cells were counted and the cell concentration was diluted to 1×10⁶/mL. 6×10⁶ cells were transferred to another tube and resuspended in a total of 6 mL of NK-MED-003 medium. The cells were transferred to 1 well of ultra-low adhesion 6-well plate (Corning, 3471) and the plate was placed on a platform shaker and rotated at 98 RPM for 18+/−2 hours (overnight).
2. At day 0, morning, at 18+/−2 hours after iPSC aggregation: The plate was rotated in a circular motion to move aggregates towards center of the well and aggregates were collected in a conical tube. Alternatively, all the aggregate solution mix was collected. Aggregates were allowed to settle for 15+/−5 minutes. Cells were resuspended in NK-MED-004 medium (Table 4). The cell number in aggregates was counted. The seeding density was adjusted as needed to resuspend 3×10⁵ cells in aggregates in 2 mL NK-MED-004 medium and plated in one well of a 6-well low adhesion plate. Alternatively, for scale up, an appropriate number of cells was resuspended and transferred to a PBS spinner vessel (PBS Biotech). Seeding density tested for PBS seeding vessel was approximately 1-1.2×10⁵ cells per mL per final media volume (day 0+8 hrs). The plate was placed on a platform shaker and rotated at 98 RPM for 8 hours or the PBS spinner vessel were placed on a PBS base (PBS-MINI MagDrive Base Unit; PBS Biotech), in CO₂ incubator with a rotation speed of RPM 38 to 39.
3. At day 0, afternoon, at 8 hours after NK-MED-004 medium addition: 2 mL per well of NK-MED-005 medium (Table 5) was added or scaled up for PBS spinner vessels. The plate was returned to platform shaker or PBS spinner vessel to its base in the CO₂ incubator and left undisturbed until day 2. NK-MED-005 medium components were 2× of their final concentration, therefore it was added to cells in NK-MED-004 at a 1:1 volume ratio.
4. At day 2: NK-MED-005 medium was replaced with NK-MED-006 medium (Table 6).
5. At day 4: NK-MED-006 medium was replaced with NK-MED-007 medium (Table 7).
6. At day 6: Starting at day 6, medium with all aggregates and single cells was transferred into an appropriate volume centrifuge conical tube. Cells were centrifuged and resuspended in NK-MED-008 medium (Table 8) and placed back into original wells and onto platform shaker, or into original vessels and onto base, and returned for continued culture.
7. At day 10: Half media change was made with NK-MED-008 medium.
8. At day 14: Full media change was made with NK-MED-009 medium (Table 9).
9. From day 17 onwards: Starting at day 17 (and at day 20, and every 2 to 3 days from day 20 onwards), single cell density was estimated from cell culture. If cell density exceeded 3×10⁶, cells were diluted to 1-2×10⁶ either by topping up cultures with fresh NK-MED-009 medium or by a complete medium change if medium color has completely turned yellow. Representative culture samples were harvested at day 6, day 10, day 14, day 17, day 20, and day 28 for FACS and TruSeq analysis to monitor differentiation of the cells.

In the tables below, the volumes are approximate to get the desired concentration.

TABLE 1
MEDIUM COMPOSITION FOR NK-MED-001

| | Working | | Stock |
|---|---|---|---|
| Component | Conc. | Volume | Conc. |
| STEMFLEX ™ Basal | 90% | 900 mL | 100% |
| (Thermo Fisher, A3349401) | | | |
| STEMFLEX ™ Supplement | 1X | 100 mL | 10X |
| (Thermo Fisher, A3349401) | | | |
| Thiazovivin | 2 μM | 200 μL | 10 mM |
| (Biological Industry, | | | |
| 1226056-71-8) | | | |

TABLE 2
MEDIUM COMPOSITION FOR NK-MED-002

| | Working | | Stock |
|---|---|---|---|
| Component | Conc. | Volume | Conc. |
| STEMFLEX ™ Basal | 90% | 900 mL | 100% |
| (Thermo Fisher, A3349401) | | | |
| STEMFLEX ™ Supplement | 1X | 100 mL | 10X |
| (Thermo Fisher, A3349401) | | | |

TABLE 3
MEDIUM COMPOSITION FOR NK-MED-003

| | Working | | | Stock |
|---|---|---|---|---|
| Component | Conc. | Volume | | Conc. |
| STEMFLEX ™ Basal | 90% | 899 | mL | 100% |
| (Thermo Fisher, A3349401) | | | | |
| STEMFLEX ™ Supplement | 1X | 100 | mL | 10X |
| (Thermo Fisher, A3349401) | | | | |
| Thiazovivin | 10 μM | 1000 | μL | 10 mM |

(Biological Industry, 1226056-71-8)

TABLE 4
Medium composition for NK-MED-004

| | Working | | | | Stock | |
|---|---|---|---|---|---|---|
| Component | Conc. | | Volume | | Conc. | |
| STEMdiff APEL 2 Medium | 100% | | 999 | mL | 100% | |
| (STEMCELL Technologies, | | | | | | |
| 05275) | | | | | | |
| rh BMP-4 (Peprotech, | 30 | ng/mL | 300 | μL | 100 | μg/mL |
| 120-05ET) | | | | | | |
| Thiazovivin | 10 | μM | 1000 | μL | 10 | mM |

(Biological Industry,
1226056-71-8)

TABLE 5
Medium composition for NK-MED-005

| | Working | | | | Stock | |
|---|---|---|---|---|---|---|
| Component | Conc. | | Volume | | Conc. | |
| STEMdiff APEL 2 Medium | 100% | | 998 | mL | 100% | |
| (STEMCELL | | | | | | |
| Technologies, 05275) | | | | | | |
| rh BMP-4 (Peprotech, | 30 | ng/mL | 300 | μL | 100 | μg/mL |
| 120-05ET) | | | | | | |
| rh FGF2 (Peprotech, | 100 | ng/mL | 1000 | μL | 100 | μg/mL |
| 100-18C-1MG) | | | | | | |
| CHIR 99021 | 6 | μM | 600 | μL | 10 | mM |
| (Selleckchem, S1263) | | | | | | |
| Activin-A | 5 | ng/mL | 100 | μL | 50 | μg/mL |

(R&D Systems,
338-AC-01M/CF

TABLE 6
Medium composition for NK-MED-006

| | Working | | | | Stock | |
|---|---|---|---|---|---|---|
| Component | Conc. | | Volume | | Conc. | |
| STEMdiff APEL 2 Medium | 100% | | 997 | L | 100% | |
| (STEMCELL | | | | | | |
| Technologies, 05275) | | | | | | |
| rh FGF2 (Peprotech, | 20 | ng/mL | 200 | μL | 100 | μg/mL |
| 100-18C-1MG) | | | | | | |
| rh VEGF165 (Peprotech, | 20 | ng/mL | 200 | μL | 100 | μg/mL |
| 100-20-1MG) | | | | | | |
| rh TPO (Peprotech, 300-18) | 20 | ng/mL | 200 | μL | 100 | μg/mL |
| rh SCF (Peprotech, 300-07) | 100 | ng/mL | 1000 | μL | 100 | μg/mL |
| rh IL-3 (Peprotech, | 40 | ng/mL | 400 | μL | 100 | μg/mL |
| 200-03-100UG) | | | | | | |
| rh Flt3L (Peprotech, | 20 | ng/mL | 200 | μL | 100 | μg/mL |
| 300-19) | | | | | | |
| WNT C-59 (Selleckchem, | 2 | μM | 200 | μL | 10 | mM |
| S7037) | | | | | | |
| SB431542 (Selleckchem, | 5 | μM | 500 | μL | 10 | mM |
| S1067) | | | | | | |

TABLE 7
Medium composition for NK-MED-007

| | Working | | Stock |
|---|---|---|---|
| Component | Conc. | Volume | Conc. |
| STEMdiff APEL 2 Medium | 100% | 998 mL | 100% |
| (STEMCELL Technologies, | | | |
| 05275) | | | |
| rh FGF2 (Peprotech, | 20 ng/mL | 200 μL | 100 μg/mL |
| 100-18C-1MG | | | |
| rh VEGF165 (Peprotech, | 20 ng/mL | 200 μL | 100 μg/mL |
| 100-20-1MG) | | | |
| rh TPO (Peprotech, 300-18) | 20 ng/mL | 200 μL | 100 μg/mL |
| rh SCF (Peprotech, 300-07) | 100 ng/mL | 1000 μL | 100 μg/mL |
| rh IL-3 (Peprotech, | 40 ng/mL | 400 μL | 100 μg/mL |
| 200-03-100UG) | | | |
| rh Flt3L (Peprotech, 300-19) | 20 ng/mL | 200 μL | 100 μg/mL |

TABLE 8
Medium composition for NK-MED-008

| Component | Working Conc. | | Volume | | Stock Conc. | |
|---|---|---|---|---|---|---|
| DMEM (high glucose, GlutaMAX) | 55.47% | | 555 | mL | 100% | |
| (Thermo Fisher, 10566016) | | | | | | |
| F-12 with GlutaMAX | 27.74% | | 277 | mL | 100% | |
| (Thermo Fisher, 31765035) | | | | | | |
| GlutaMAX (Thermo Fisher, 35050079) | 1X | | 10 | mL | 100X | |
| Glucose* | 10.25 | mM | 4.1 | mL | 2500 | mM |
| Human AB serum | 15% | | 150 | mL | 100% | |
| (Valley Biomedical Inc, HP1022) | | | | | | |
| Zinc sulfate (Millipore Sigma, Z0251) | 37 | μM | 978 | μL | 37 | mM |
| Ethanolamine (Millipore Sigma, E0135) | 50 | μM | 3 | μL | 16.6M | |
| Ascorbic acid | 20 | μg/mL | 2000 | μL | 10 | mg/mL |
| (Fisher Scientific, NC0762606) | | | | | | |
| Sodium selenite | 5 | ng/mL | 50 | μL | 100 | μg/mL |
| (Millipore Sigma, S9133-1MG) | | | | | | |
| β-mercaptoethanol | 1 | μM | 18 | μL | 55 | mM |
| (Thermo Fisher, 21985-023) | | | | | | |
| rh IL-3 (Peprotech, 200-03-100UG) | 5 | ng/mL | 50 | μL | 100 | μg/mL |
| rh IL-7 (Peprotech, 200-07) | 20 | ng/mL | 200 | μL | 100 | μg/mL |
| rh Flt3L (Peprotech, 300-19) | 15 | ng/mL | 150 | μL | 100 | μg/mL |
| rh IL-15 (Peprotech, 200-15) | 15 | ng/mL | 150 | μL | 100 | μg/mL |
| rh SCF (Peprotech, 300-07) | 20 | ng/mL | 200 | μL | 100 | μg/mL |

*Total glucose concentration in medium is 27 mM (accounting for glucose in DMEM medium, F12 supplement and added glucose provided here).

TABLE 9
Medium composition for NK-MED-009

| Component | Working Conc. | | Volume | | Stock Conc. | |
|---|---|---|---|---|---|---|
| DMEM (high glucose, GlutaMAX) | 55.47% | | 555 | mL | 100% | |
| (Thermo Fisher, 10566016) | | | | | | |
| F-12 with GlutaMAX | 27.74% | | 277 | mL | 100% | |
| (Thermo Fisher, 31765035) | | | | | | |
| GlutaMAX (Thermo Fisher, 35050079) | 1X | | 10 | mL | 100X | |
| Glucose* | 10.25 | mM | 4.1 | mL | 2500 | mM |
| Human AB serum | 15% | | 150 | mL | 100% | |
| (Valley Biomedical Inc, HP1022) | | | | | | |
| Zinc sulfate (Millipore Sigma, Z0251) | 37 | μM | 978 | μL | 37 | mM |
| Ethanolamine | 50 | μM | 3 | μL | 16.6M | |
| (Millipore Sigma, E0135) | | | | | | |
| Ascorbic acid | 20 | μg/mL | 2000 | μL | 10 | mg/mL |
| (Fisher Scientific, NC0762606) | | | | | | |
| Sodium selenite | 5 | ng/mL | 50 | μL | 100 | μg/mL |
| (Millipore Sigma, S9133-1MG) | | | | | | |
| β-mercaptoethanol | 1 | μM | 18 | μL | 55 | mM |
| (Thermo Fisher, 21985-023) | | | | | | |
| rh IL-7 (Peprotech, 200-07) | 20 | ng/mL | 200 | μL | 100 | μg/mL |
| rh Flt3L (Peprotech, 300-19) | 15 | ng/mL | 150 | μL | 100 | μg/mL |
| rh IL-15 (Peprotech, 200-15) | 15 | ng/mL | 150 | μL | 100 | μg/mL |
| rh SCF (Peprotech, 300-07) | 20 | ng/mL | 200 | μL | 100 | μg/mL |

*Total glucose concentration in medium is 27 mM (accounting for glucose in DMEM medium, F12 supplement and added glucose provided here).

### Example 3

Differentiating Stem Cells into Natural Killer Cells-Protocol 1.5

Protocol 1.5 was utilized to differentiate stem cells, such as wild-type and/or edited iPS cells, into hematopoietic stem and progenitor cells (HSPCs) and then into natural killer (NK) cells. iPS cells were cultured in STEMFLEX™ (SF) (Thermo Fisher, A3349401) media prior to beginning differentiation. iPS cells were differentiated using the following steps. Media used throughout is shown in Tables 10-11:
Day −1 (afternoon): STEMFLEX™ media (SF) was aspirated and cells were washed with DPBS (no calcium, no magnesium) (Thermo Fisher Scientific, 14190250). DPBS was aspirated and 2 mL pre-warmed ACCUTASE® (Innovative Cell Technologies, AT-104) was added per flask (scale up if needed: 80 μL of ACCUTASE® per 1 cm²). Cells were incubated at 37° C. for 3-5 minutes or until all the colonies detached. Accutase digested cells were diluted with SF for a ratio of 3:1 (SF:ACCUTASE®). Cells were gently pipetted 2-3 times with a serological pipet until cells detached. Cells were transferred to a conical tube and the plate was rinsed with SF, the rinse was added to the same tube. Enough SF was added to cells to dilute the ACCUTASE® to a ratio of 4:1 (SF:ACCUTASE®). Cells were spun for 5 minutes at 300 g. Supernatant was aspirated and cells were re-suspended in SF. Cells were counted. The cell concentration was adjusted to 1×10⁶/mL by transferring 6×10⁶ cells to another tube, resuspended in total 6 mL of NK-MED-003 medium. Cells were transferred to 1 well of ultra-low adhesion 6-well plate (Corning, 3471). The plate was placed on a horizontal orbital shaker.
2. Day 0 (morning): 16 hours later: Start differentiation: The plate was rotated in a circular motion to move aggregates towards center of the well, and aggregates were collected and transferred to a conical tube. The aggregates were allowed to settle. Aggregates were resuspended in NK-MED-004 medium (2 mL per aggregated well). Cell number in aggregates was counted. The cells in aggregates density was adjusted by resuspending 3×10⁵ cells in aggregates in 2 mL APEL-B media and plated in 1 well of a 6-well low adhesion plate. The plate was placed on a horizontal orbital shaker and rotated for 8 hours.
3. Day 0 (afternoon, following 8 hours of pre-incubation): 2 mL per well of NK-MED-005 medium was added per well. The plate was returned to the orbital shaker and left untouched until day 2.
4. Day 2: NK-MED-006 was replaced with A-FVTSIF-SW media.
5. Day 4: NK-MED-007 media was replaced with A-FVTSIF media.
6. Day 6: Using this method, single cells (HSPCs) started emerging at day 5-6. Media with all embryoid bodies (EBs) and single cells were transferred into an appropriate volume centrifuge conical tube and centrifuged. For 6-well plates: EBs from each well were resuspended in 3 mL of DF-NK+IL3 media (Table 10) and transferred into their original wells. The plate was returned to the orbital shaker.
7. Day 10:6-well plates: 3 mL of DF-NK+IL3 media was added to each well on top of the original media and then returned to orbital shaker.
8. Day 14: Full media change. Transfer cells to DF-NK media (Table 11), no IL3 was added from this point. Media with all EBs and single cells was transferred into a centrifuge conical tube and centrifuged. Supernatant was removed. 6-well plates: EBs from each well were resuspended in 3 mL of DF-NK media and transferred into their original wells.
9. Days 14-28: Every 3-4 days media was topped off with 3 mL of fresh DF-NK media, then 3-4 days later spent media was replaced with 3 mL of fresh DF-NK media by collecting the cells in a conical tube and centrifuging. Representative culture samples were harvested at days 6, 10, 14, 21, 28 and 35 for FACS and TruSeq analysis to monitor differentiation of the cells.

TABLE 10
DF-NK + IL3 MEDIA

| Component | Working Conc. | Volume | Stock Conc. | Vendor | Item# |
|---|---|---|---|---|---|
| DMEM | Ratio 2 to F12 | 558 | 100% | Thermo Fisher | 10566016 |
| (high glucose, | | | | Scientific | |
| GlutaMAX) | | | | | |
| F-12 with | Ratio 1 to DMEM | 279 | 100% | Thermo Fisher | 31765035 |
| GlutaMAX | | | | Scientific | |
| GlutaMAX | 1X | 10 mL | 100X | | |
| Human AB | 15% | 150 mL | 100% | Valley Biomedical Inc | HP1022 |
| serum | | | | | |
| Ascorbic acid | 20 μg/mL | 2000 μL | 10 mg/mL | MilliporeSigma | |
| Sodium | 5 ng/mL | 50 μL | 100 μg/mL | MilliporeSigma | |
| selenite | | | | | |
| rh IL-3 | 5 ng/mL | 50 μL | 100 μg/mL | PeproTech | 200-03 |
| rh IL-7 | 20 ng/mL | 200 μL | 100 μg/mL | PeproTech | 200-07 |
| rh Flt3L | 15 ng/mL | 150 μL | 100 μg/mL | PeproTech | 300-19 |
| rh IL-15 | 15 ng/mL | 150 μL | 100 μg/mL | PeproTech | 200-15 |
| rh SCF | 20 ng/mL | 200 μL | 100 μg/mL | PeproTech | 300-07 |

TABLE 11
DF-NK MEDIA

| | Working | | | | |
|---|---|---|---|---|---|
| Component | Conc. | Volume | Stock Conc. | Vendor | Item# |
| DMEM | Ratio 2 to F12 | 558 | 100% | Thermo Fisher | 10566016 |
| (high glucose, | | | | Scientific | |
| GlutaMAX) | | | | | |
| F-12 with | Ratio 1 to | 279 | 100% | Thermo Fisher | 31765035 |
| GlutaMAX | DMEM | | | Scientific | |
| GlutaMAX | 1X | 10 mL | 100X | | |
| Human AB serum | 15% | 150 mL | 100% | Valley Biomedical Inc | HP1022 |
| Ascorbic acid | 20 μg/mL | 2000 μL | 10 mg/mL | MilliporeSigma | |
| Sodium selenite | 5 ng/mL | 50 μL | 100 μg/mL | MilliporeSigma | |
| rh IL-3 | | | | | |
| rh IL-7 | 20 ng/mL | 200 μL | 100 μg/mL | PeproTech | 200-07 |
| rh Flt3L | 15 ng/mL | 150 μL | 100 μg/mL | PeproTech | 300-19 |
| rh IL-15 | 15 ng/mL | 150 μL | 100 μg/mL | PeproTech | 200-15 |
| rh SCF | 20 ng/mL | 200 μL | 100 μg/mL | PeproTech | 300-07 |

### Example 4

Generation of ADAM17-Null Human Pluripotent Stem Cells (hPSCs) Guide RNA (gRNA) Selection for ADAM17 in hPSCs

Ten ADAM17 targeting gRNAs were designed for targeting exon 1 of the ADAM17 coding sequence. These gRNAs had predicted low off-target scores based on sequence homology prediction using gRNA design software. The target sequences of the gRNAs with the corresponding PAMs are presented in Table 12. In some embodiments, the gRNA comprises an RNA sequence corresponding to the target DNA sequence.

TABLE 12
ADAM17 gRNA TARGET SEQUENCES

| Name | Target Sequence (5′-3′) | SEQ ID NO: | PAM |
|---|---|---|---|
| ADAM17 Ex1_T2 | GGTCGCGGCGCCAGCACGAA | 1 | AGG |
| ADAM17 Ex1_T4 | CCGAAGCCCGGGTCATCCGG | 2 | AGG |
| ADAM17 Ex1_T9 | CCGCGACCTCCGGATGACCC | 3 | GGG |
| ADAM17 Ex1_T10 | CGTGCTGGCGCCGCGACCTC | 4 | CGG |
| ADAM17 Ex1_T11 | CGAAAGGAACCACGCTGGTC | 5 | AGG |
| ADAM17 Ex1_T12 | CAGCGTGGTTCCTTTCGTGC | 6 | TGG |
| ADAM17 Ex1_T19 | GCCGCGACCTCCGGATGACC | 7 | CGG |
| ADAM17 Ex1_T24 | GAACCACGCTGGTCAGGAAT | 8 | AGG |
| ADAM17 Ex1_T25 | CAGCACGAAAGGAACCACGC | 9 | TGG |
| ADAM17 Ex1_T28 | GTAGCGGGGCCGGGAACATG | 10 | AGG |

To assess their cutting efficiency in hPSCs, iPS cells were electroporated using the Neon Electroporator (Neon Transfection Kit ThermoFisher Cat #MPK5000) with a ribonucleoprotein (RNP) mixture of Cas9 protein (Biomay) and guide RNA (IDT) (See Table 12 for gRNA target sequences) at a molar ratio of 5:1 (gRNA: Cas9) with absolute values of 125 pmol Cas9 and 625 μmol gRNA. To form the RNP complex, gRNA and Cas9 were combined in one vessel with R-buffer (Neon Transfection Kit) to a total volume of 25 μL and incubated for 15 min at RT. Cells were dissociated using ACCUTASE®, then resuspended in STEMFLEX™ media (Gibco, cat #11320033), counted using an NC-200 (ChemoMetec) and centrifuged. A total of 1×10⁶ cells were resuspended with the RNP complex and R-buffer was added to a total volume of 125 μL. This mixture was then electroporated with 1 pulse for 20 ms at 1500 V and 1 pulse for 100 ms at 500 V. Following electroporation, the cells were pipetted out into an Eppendorf tube filled with STEMFLEX™ media with REVITACELL™ Supplement (100×). This cell suspension was then plated into tissue culture dishes pre-coated with BIOLAMININ 521 CTG at 1:20 dilution. Cells were cultured in a normoxia incubator (37° C., 8% CO₂) for 48 hours. After 48 hours, genomic DNA was harvested from the cells using QuickExtract (Lucigen, Middleton, WI; Cat #QE09050).

PCR for the target ADAM17 sequence was performed and the resulting amplified DNA was assessed for cutting efficiency by TIDE analysis. PCR for relevant regions was performed using Platinum Taq Supermix (Invitrogen, cat #125320176 and Cat #11495017). The sequences of the PCR primers are presented in Table 13; and the cycling conditions are provided in Table 14.

TABLE 13
ADAM17 TIDE/INDEL PRIMERS

| | | | SEQ |
|---|---|---|---|
| Name | Type | Sequence (5′-3′) | ID NO: |
| ADAM17 F2 | forward | AGAATCTTCCCAGTAGGCGG | 11 |
| ADAM17 R2 | reverse | CTCAGGCGCTCAGTCACTAC | 12 |

TABLE 14
ADAM17 PCR/INDEL PCR CYCLING PARAMETERS

| Step | Temperature | Time | | Cycles |
|---|---|---|---|---|
| Denaturation | 94° C. | 2 | min | 1 |
| Denaturation | 94° C. | 15 | sec | 34 |
| Annealing | 57° C. | 30 | sec | |
| Extension | 68° C. | 45 | sec | |
| Elongation | 68° C. | 5 | min | 1 |

| | | |
|---|---|---|
| Hold | 4° C. | hold |

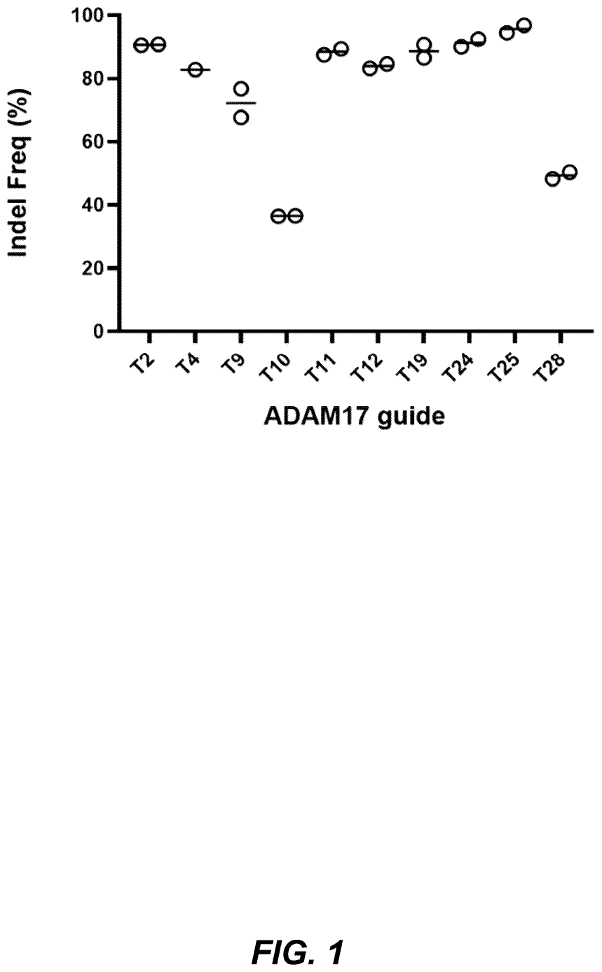

The resulting amplicons were submitted for PCR cleanup and Sanger sequencing. Sanger sequencing results were input into Tsunami software along with the guide sequence. Indel percentages and identities were calculated by the software. Particular gRNAs were then selected based on their indel frequency in hPSCs. FIG. 1 shows the cutting efficiency of 10 ADAM17 guides. ADAM17 Ex1_T2 was chosen for further clone generation due to its high on-target activity.

ADAM17 KO hPSC Clone Generation and Characterization

Using ADAM17 T2 gRNA, iPSCs were electroporated and single-cell sorted 3 days post electroporation using the WOLF FACS-sorter (Nanocellect) into BIOLAMININ 521 CTG coated 96-well plates with STEMFLEX™ and REVITACELL™ Supplement (100×). Plated single cells were grown in a normoxia incubator (37° C., 8% CO₂) with every other day media changes until colonies were large enough to be re-seeded as single cells. When confluent, samples were split for maintenance and genomic DNA extraction.

The ADAM17 KO state of clones was confirmed via PCR and Sanger sequencing, as described above. The resulting DNA sequences of the target ADAM17 region were aligned in Snapgene software to determine indel identity and homo- or heterozygosity. Karyotypic status of clones was evaluated through Cell Line Genetics service (Madison, WI) and normal karyotype was reported.

### Example 5

Generation and Selection of CIITA gRNA
Guide RNA (gRNA) Selection for CIITA in hPSCs

5 CIITA targeting gRNAs were designed for targeting exons 2 and 3 of the CIITA coding sequence. These gRNAs had predicted low off-target scores based on sequence homology prediction using gRNA design software. The target sequences of the gRNAs are presented in Table 15. In some embodiments, the gRNA comprises an RNA sequence corresponding to the target DNA sequence.

TABLE 15
CIITA gRNA TARGET SEQUENCES

| | | SEQ | |
|---|---|---|---|
| Name | Target Sequence (5′-3′) | ID NO: | PAM |
| CIITA Ex3_T6 | GGTCCATCTGGTCATAGAAG | 13 | TGG |
| CIITA Ex3_T16 | GCTCCAGGTAGCCACCTTCT | 14 | AGG |
| CIITA Ex3_T20 | TAGGGGCCCCAACTCCATGG | 15 | TGG |
| CIITA Ex4_T1 | GGCTTATGCCAATATCGGTG | 16 | AGG |
| CIITA Ex4_T25 | AGGTGATGAAGAGACCAGGG | 17 | AGG |

To assess their cutting efficiency in hPSCs, human embryonic stem cells were electroporated using the Neon Electroporator (Neon Transfection Kit ThermoFisher Cat #MPK5000) with a ribonucleoprotein (RNP) mixture of Cas9 protein (Biomay) and guide RNA (Agilent) (See Table 15 for gRNA target sequences) at a molar ratio of 5:1 (gRNA:Cas9) with absolute values of 125 pmol Cas9 and 625 μmol gRNA. To form the RNP complex, gRNA and Cas9 were combined in one vessel with R-buffer (Neon Transfection Kit) to a total volume of 25 μL and incubated for 15 min at RT. Cells were dissociated using ACCUTASER, then resuspended in STEMFLEX™ media (Gibco, cat #11320033), counted using an NC-200 (ChemoMetec) and centrifuged. A total of 1×10⁶ cells were resuspended with the RNP complex and R-buffer was added to a total volume of 125 μL. This mixture was then electroporated with 3 pulses for 30 ms at 1100 V. Following electroporation, the cells were pipetted out into an Eppendorf tube filled with STEMFLEX™ media with RevitaCell™. This cell suspension was then plated into tissue culture dishes pre-coated with BIOLAMININ 521 CTG at 1:20 dilution. Cells were cultured in a normoxia incubator (37° C., 8% CO₂) for 48 hours. After 48 hours, genomic DNA was harvested from the cells using QuickExtract (Lucigen, Middleton, WI; Cat #QE09050).

PCR for the target CIITA sequence was performed and the resulting amplified DNA was assessed for cutting efficiency by TIDE analysis. PCR for relevant regions was performed using Platinum Taq Supermix (Invitrogen, cat #125320176 and Cat #11495017). The sequences of the PCR primers are presented in Table 16; and the cycling conditions provided in Table 17.

TABLE 16
CIITA TIDE/INDEL PRIMERS

| | | | SEQ |
|---|---|---|---|
| Name | Type | Sequence (5′-3′) | ID NO: |
| CIITA F5 | forward | TCCTGACTCTCTGGTGTGAGAT | 18 |
| CIITA R5 | reverse | CAGAGAGCGTCCCACAGAC | 19 |

TABLE 17
CIITA PCR/INDEL CYCLING PARAMETERS

| Step | Temperature | Time | | Cycles |
|---|---|---|---|---|
| Denaturation | 94° C. | 2 | min | 1 |
| Denaturation | 94° C. | 15 | sec | 34 |
| Annealing | 57° C. | 30 | sec | |
| Extension | 68° C. | 45 | sec | |
| Elongation | 68° C. | 5 | min | 1 |

| | | |
|---|---|---|
| Hold | 4° C. | hold |

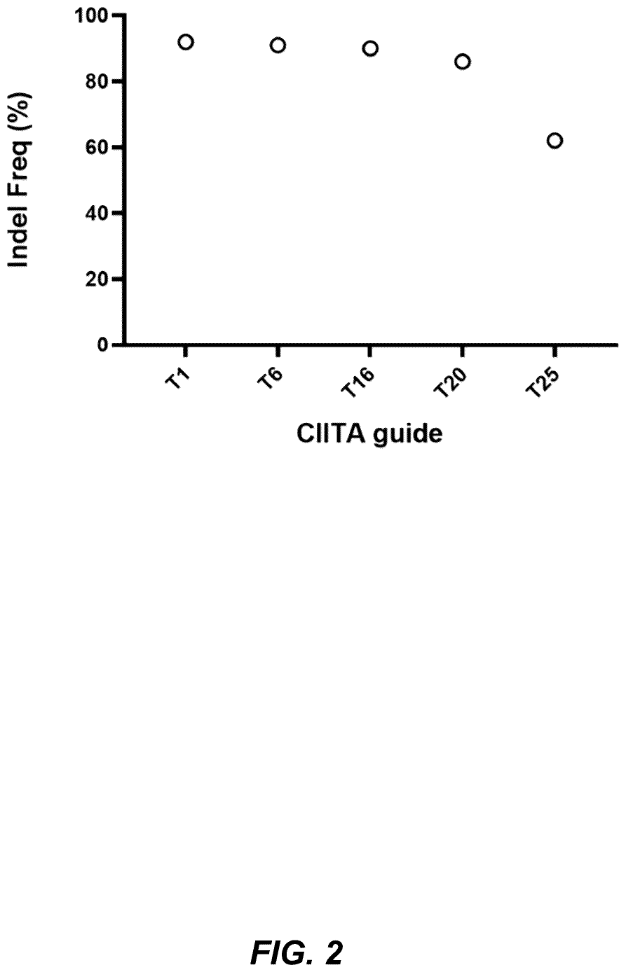

The resulting amplicons were submitted for PCR cleanup and Sanger sequencing. Sanger sequencing results were input into Tsunami software along with the guide sequence. Indel percentages and identities were calculated by the software. Particular gRNAs were then selected based on their indel frequency in hPSCs. FIG. 2 shows the cutting efficiency of 5 CIITA guides.

Off-targets of the selected gRNAs were assessed in the stem cell-derived DNA using hybrid capture analysis of the sequence similarity predicted sites. CIITA Ex3 T6 and CIITA Ex4_T1 guides did not show detectable off-target effects. CIITA T6 gRNA was chosen for further clone generation due to its high on-target activity and undetectable off-target activity.

### Example 6

Generation of CAR Knock-In, CIITA Null Human Pluripotent Stem Cells (hPSCs) Design of CIITA KO, CAR KI Strategy

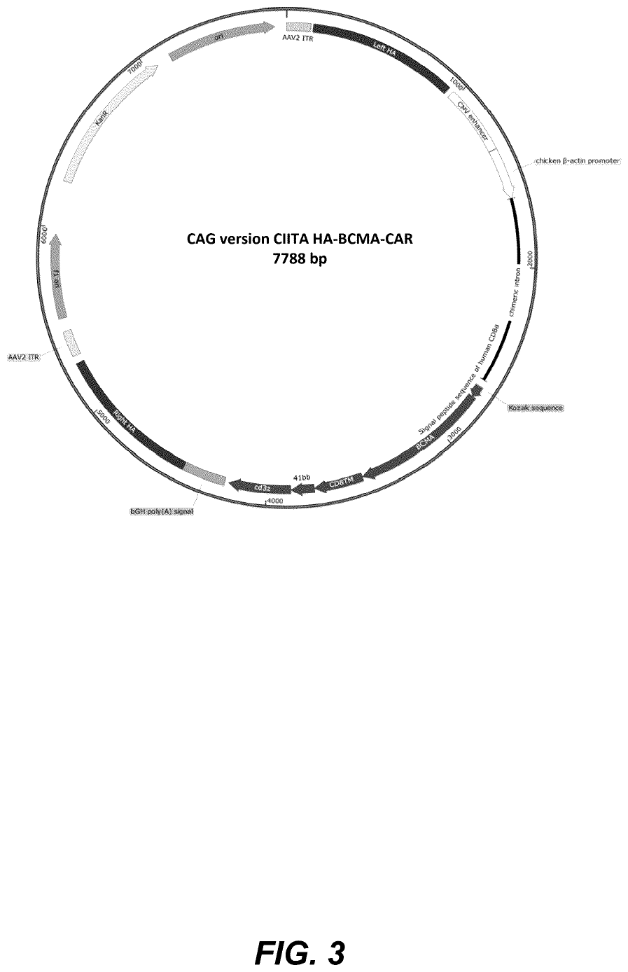

Plasmid design to insert a CAR sequence, such as a BCMA CAR sequence, into the CIITA gene locus was made such that 86 base pairs (bp) of the CIITA exon 2 (gccaccatggagttggggcccctagaaggtggctacctggagcttcttaaca gcgatgctgaccccctgtgcctctaccacttcta (SEQ ID NO: 20)) would be removed after undergoing homology directed repair (HDR). The removal of this portion of CIITA would result in a frame shift of the CIITA coding sequence (CDS) nullifying the expression of functional CIITA protein. Successful HDR would also result in the insertion of the CAR sequence into the genome. The donor plasmid contained a CAGGS promoter driven cDNA of a CAR sequence flanked by 800 base pair homology arms with identical sequence to the CIITA gene locus around exon 2. FIG. 3 presents a schematic of an example BCMA CAR encoding plasmid (SEQ ID NO: 66) and Table 18 identifies the elements and locations therein.

TABLE 18
ELEMENTS OF BCMA CAR DONOR PLASMID

| Element | Location (size in bp) | SEQ ID NO: |
|---|---|---|
| Left ITR | 1-130 (130) | 21 |
| LHA-CIITA | 145-944 (800) | 22 |
| CMV enhancer | 973-1352 (380) | 23 |
| chicken β-actin promoter | 1355-1630 (276) | 24 |
| chimeric intron | 1631-2639 (1009) | 25 |
| CD8a signal peptide | 2684-2746 (63) | 26 |
| BCMA targeting fragment | 2747-3481 (735) | 27 |
| CD8TM | 3482-3745 (264) | 28 |
| 41BB co-stim domain | 3746-3871 (126) | 29 |
| CD3Z domain | 3872-4207 (336) | 30 |
| bGH poly(A) signal | 4229-4453 (225) | 31 |
| RHA-CIITA | 4460-5259 (800) | 32 |
| Right ITR | 5301-5441 (141) | 33 |

The CIITA-T6 gRNA (comprising a spacer sequence of SEQ ID NO: 175) was used to facilitate insertion of the BCMA CAR transgene at the targeted CIITA gene locus. The target sequence of CIITA-T6 is not present in the donor plasmid and thus the donor plasmid itself would not be targeted by this gRNA. CIITA-T6 induced CRISPR cutting in the human genome at exon 2 of CIITA would lead to a frameshift and loss of CIITA protein. The BCMA CAR donor plasmid was introduced along with the ribonucleoprotein (RNP) complex made up of the CIITA targeting gRNA and Cas9 protein. Per 1 million of human embryonic stem cells, 4 μg of plasmid DNA was delivered along with the RNP via electroporation. Electroporation was carried out in hiPSC cells using the Neon Electroporator (Neon Transfection Kit ThermoFisher Cat #MPK5000) with the RNP mixture of Cas9 protein (Biomay) and guide RNA (Synthego) at a molar ratio of 5:1 (gRNA: Cas9) with absolute values of 125 μmol Cas9 and 625 μmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in STEMFLEX™ media, counted using an NC-200 (ChemoMetec) and centrifuged. A total of 2×10⁶ cells were resuspended with the RNP complex and R-buffer was added to a total volume of 115 μL. This mixture was then electroporated with 1 pulse for 20 ms at 1500 V followed by 1 pulse for 100 ms at 500 V. Following electroporation, the cells were pipetted out into a well of a 6 well plate filled with STEMFLEX™ media with REVITACELL™ Supplement (100×) and laminin 511. The plates were pre-coated with BIOLAMININ 521 CTG at 1:10 dilution. Cells were cultured in a normoxia incubator (37° C., 8% CO₂).

Seven to ten days post electroporation, the cells were enriched for BCMA CAR expressing cells using an antibody against BCMA CAR (15C04-APC or 15C04-PE) via magnetic assisted cell sorting (MACS) using anti-mouse IgG Dynabeads (ThermoFisher, CELLection™ Pan Mouse IgG Kit, 11531D). These enriched cells represent a bulk KI population of BCMA-CAR positive cells.

TABLE 19
gRNA TARGET SEQUENCES

| | Target | SEQ | |
|---|---|---|---|
| Name | Sequence (5′-3′) | ID NO: | PAM |
| CIITA Ex3_T6 | GGTCCATCTGGTCATAGAAG | 13 | TGG |
| gRNA | | | |
| B2M-2 gRNA | GGCCGAGATGTCTCGCTCCG | 34 | TGG |
| (Exon 1_T2) | | | |
| ADAM17 Ex1_T2 | GGTCGCGGCGCCAGCACGAA | 1 | AGG |
| gRNA | | | |

### Example 7

Generation of IL15/IL15Rα-P2A-HLA-E Trimer Knock-in, BCMA CAR Knock-in, CIITA Null, B2M Null Human Pluripotent Stem Cells (hPSCs)

### Design of B2M KO, IL15/IL15Rα-P2A-HLA-E Trimer KI Strategy

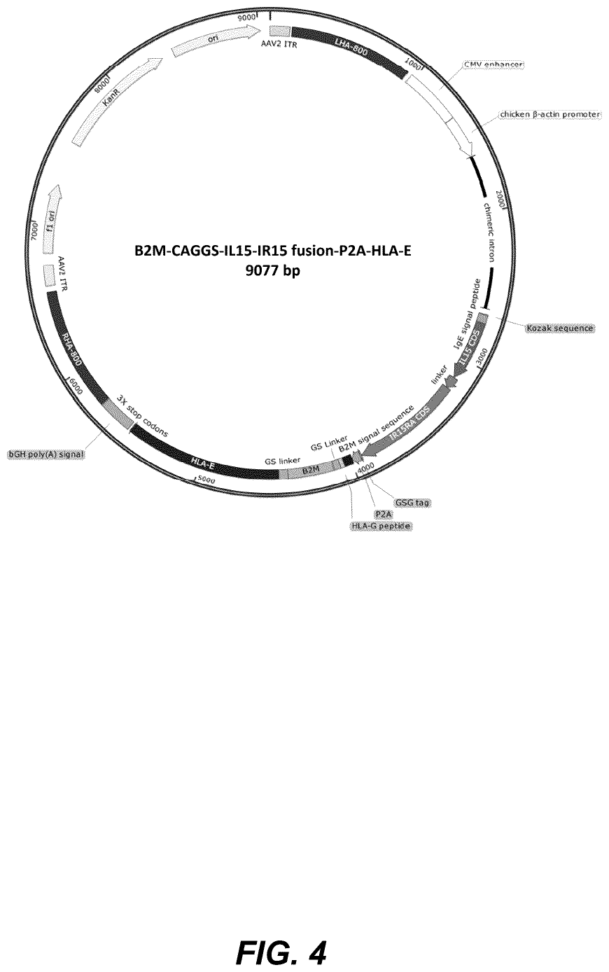

Plasmid design to insert IL15/IL15Rα-P2A-HLA-E trimer into the B2M gene locus was made such that the starting codon of B2M was removed after undergoing homology directed repair (HDR) to insert IL15/IL15Rα-P2A-HLA-E trimer, nullifying any chance of partial B2M expression. FIG. 4 presents a schematic of the plasmid SEQ ID NO: 67 and Table 20 identifies the elements and locations therein. The donor plasmid contained a CAGGS promoter driven cDNA of IL15/IL15Rα-P2A-HLA-E trimer flanked by 800 base pair homology arms with identical sequence to the B2M gene locus around exon 1.

The IL15/IL15Rα fusion sequence was designed as previously published (Hurton et al. (2016) Proc Natl Acad Sci USA.; 113 (48): E7788-E7797. doi: 10.1073/pnas. 1610544113.) The IL15/IL15Rα fusion coding sequence (including linkers) is SEQ ID NO: 76 (i.e., SEQ ID NOs: 40, 41, 42, 43, 44).

The HLA-E trimer cDNA was composed of a B2M signal peptide fused to an HLA-G presentation peptide fused to the B2M membrane protein fused to the HLA-E protein without its signal peptide. The HLA-E trimer coding sequence (including linkers) is SEQ ID NO: 75 (i.e., SEQ ID NOs: 46, 47, 48, 49, 50, and 51). This trimer design has been previously published (Gornalusse et al. (2017) Nat. Biotechnol. 35 (8): 765-772).

The P2A peptide sequence connecting IL15/IL15Rα fusion and the HLA-E trimer allows for the separate expression of both proteins from the single mRNA.

TABLE 20
ELEMENTS OF IL15/IL15Rα-
P2A-HLA-E TRIMER DONOR PLASMID

| Element | Location (size in bp) | SEQ ID NO: |
|---|---|---|
| Left ITR | 1-130 (130) | 21 |
| LHA-B2M | 145-944 (800) | 36 |
| CMV enhancer | 973-1352 (380) | 23 |
| chicken β-actin promoter | 1355-1630 (276) | 24 |
| chimeric intron | 1631-2639 (1009) | 25 |
| IgE signal peptide | 2684-2737 (54) | 40 |
| IL15 CDS | 2738-3136 (399) | 41 |
| linker | 3137-3214 (78) | 42 |
| IR15Rα CDS | 3215-3925 (711) | 43 |
| GSG tag | 3926-3934 (9) | 44 |
| P2A | 3935-3991 (57) | 45 |
| B2M signal sequence | 3992-4051 (60) | 46 |
| HLA-G peptide | 4052-4078 (27) | 47 |
| GS linker | 4079-4123 (45) | 48 |
| B2M | 4124-4420 (297) | 49 |
| GS linker | 4421-4480 (60) | 50 |
| HLA-E | 4481-5491 (1011) | 51 |
| 3X Stop codons | 5492-5500 (9) | 52 |
| bGH poly(A) signal | 5518-5742 (225) | 31 |
| RHA-B2M | 5749-6548 (800) | 54 |
| Right ITR | 6590-6730 (141) | 33 |

To insert the IL15/IL15Rα-P2A-HLA-E trimer sequence into hiPSCs, BCMA CAR-enriched hiPSCs were produced, as described in Example 6. This population was first electroporated with donor plasmid only (without CRISPR editing reagents) one day prior to a second electroporation. In the first electroporation, the Neon Electroporator was used to deliver 1 μg of donor plasmid DNA per 1 million of hiPSCs. The cells were dissociated using ACCUTASE®, then resuspended in STEMFLEX™ media, counted using an NC-200 (ChemoMetec) and centrifuged. A total of 24×10⁶ cells were resuspended with R-buffer and donor plasmid DNA to a total volume of ~600 μL. This mixture was then electroporated with 1 pulse for 20 ms at 1500 V followed by 1 pulse for 100 ms at 500 V. A total of 6 electroporations were performed and the cells were pipetted out into a 6 well plate filled with STEMFLEX™ media with REVITACELL™ Supplement (100×) and laminin 511. Cells were cultured in a normoxia incubator (37° C., 8% CO₂).

The following day, these cells were dissociated from the plate and electroporated again using additional reagents. The B2M-2 gRNA (target sequence shown in Table 19) was used to facilitate the insertion of the IL15/IL15Rα-P2A-HLA-E trimer transgene at the targeted B2M gene locus. The IL15/IL15Rα-P2A-HLA-E trimer donor plasmid was introduced along with the ribonucleoprotein (RNP) complex made up of the B2M targeting gRNA and Cas9 protein. Per 1 million of hiPSC cells, 2 μg of plasmid DNA was delivered along with the RNP via electroporation. Electroporation was carried out in hiPSC cells using the Neon Electroporator with the RNP mixture of Cas9 protein (Biomay) and guide RNA (Biospring) at a molar ratio of 5:1 (gRNA: Cas9) with absolute values of 62.5 μmol Cas9 and 312.5 μmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in STEMFLEX™ media, counted using an NC-200 (ChemoMetec) and centrifuged. A total of 7×10⁶ cells were resuspended with the RNP complex and R-buffer was added to a total volume of ~300 μL. This mixture was then electroporated with 1 pulse for 20 ms at 1500 V followed by 1 pulse for 100 ms at 500 V. A total of 3 electroporations were performed. Following electroporation, the cells were pipetted out into 2 wells of a 6 well plate filled with STEMFLEX™ media with REVITACELL™ Supplement (100×) and laminin 511. Cells were cultured in a normoxia incubator (37° C., 8% CO₂).

Seven to ten days post electroporation, the cells were enriched for HLA-E trimer expressing cells using an antibody against HLA-E (see Table 21) via magnetic assisted cell sorting (MACS) using anti-mouse IgG Dynabeads (ThermoFisher, CELLection™ Pan Mouse IgG Kit, 11531D). These enriched cells represent a bulk KI population of IL15/IL15Rα-P2A-HLA-E trimer positive cells.

TABLE 21
ANTIBODIES FOR FLOW CYTOMETRY

| Antigen | Clone | Fluorophore | Manufacturer | Catalog # |
|---|---|---|---|---|
| BCMA CAR | 15C04 | PE or APC | CRISPRtx | Custom |
| IL15 | 34559 | PE | ThermoFisher | MA5-23561 |
| B2M | 2M2 | PE | Biolegend | 316305 |
| HLA-ABC | W6/32 | Alexa 488 | Biolegend | 311415 |
| mIgG1 kappa | N/A | PE | BD Bioscience | 555749 |
| PD-L1 | B7-H1 | Alexa-488 | ThermoFisher | 53-5983-42 |
| HLA-E | 3D12 | PE | ThermoFisher | 12-9953-42 |
| HLA-E | 3D12 | APC | ThermoFisher | 17-9953-42 |

### Example 8

Generation and Characterization of IL15/IL15Rα-P2A-HLA-E Trimer Knock-In, B2M Null Human Pluripotent Stem Cells (hPSCs)

The IL15/IL15Rα-P2A-HLA-E trimer sequence, as described in Example 7, was inserted into an hiPSC line. B2M-2 gRNA (target sequence shown in Table 19) was used to facilitate the insertion of the IL15/IL15Rα-P2A-HLA-E trimer transgene at the targeted B2M gene locus. The IL15/IL15Rα-P2A-HLA-E trimer donor plasmid was introduced along with the ribonucleoprotein (RNP) complex made up of the B2M targeting gRNA and Cas9 protein. Per 1 million of hiPSC cells, 2 μg of plasmid DNA was delivered along with the RNP via electroporation. Electroporation was carried out in hiPSC cells using the Neon Electroporator with the RNP mixture of Cas9 protein (Biomay) and guide RNA (Biospring) at a molar ratio of 10:1 (gRNA: Cas9) with absolute values of 62.5 μmol Cas9 and 625 μmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in STEMFLEX™ media, counted using an NC-200 (ChemoMetec) and centrifuged. A total of 2×10⁶ cells were resuspended with the RNP complex and R-buffer was added to a total volume of ~115 μL. This mixture was then electroporated with 1 pulse for 20 ms at 1500 V followed by 1 pulse for 100 ms at 500 V. One electroporation was performed. Following electroporation, the cells were pipetted out into a well of a 6 well plate filled with STEMFLEX™ media with REVITACELL™ Supplement (100×) and laminin 511. The plates were pre-coated with BIOLAMININ 521 CTG at 1:10 dilution. Cells were cultured in a normoxia incubator (37° C., 8% CO₂).

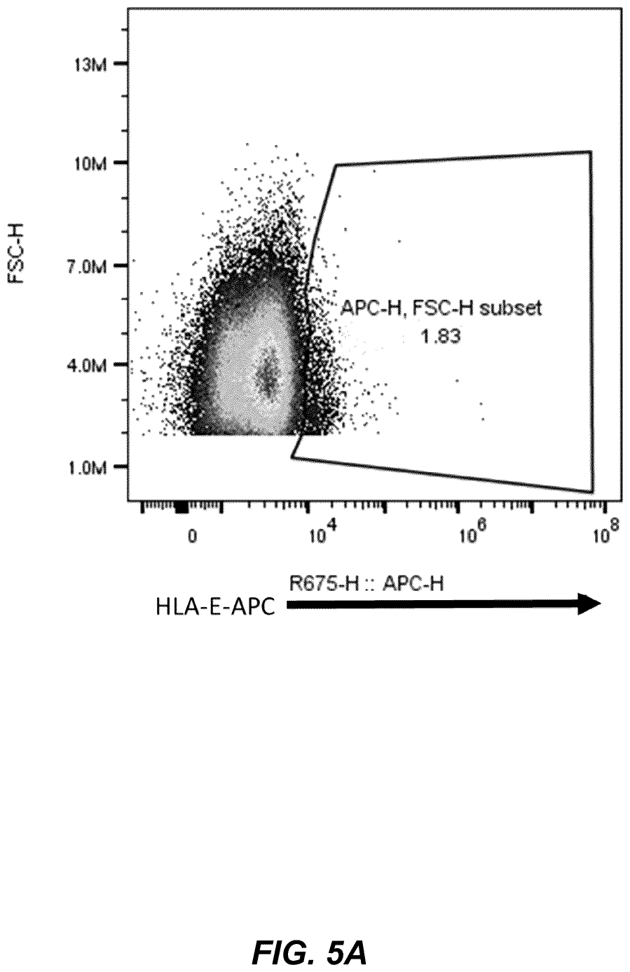

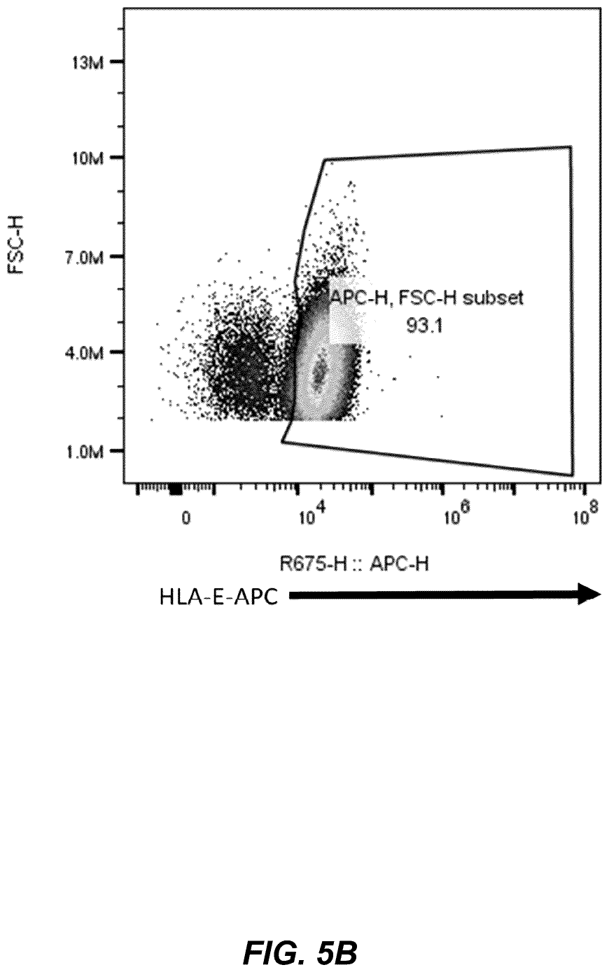

Seven to ten days post electroporation, the cells were enriched for HLA-E trimer expressing cells using an antibody against HLA-E (see Table 21) via magnetic assisted cell sorting (MACS) using anti-mouse IgG Dynabeads (ThermoFisher, CELLection™ Pan Mouse IgG Kit, 11531D). These enriched cells represent a bulk KI population of IL15/IL15Rα-P2A-HLA-E trimer positive cells. This population was assessed for HLA-E expression by flow cytometry, showing >90% HLA-E expression (FIG. 5B). WT iPSCs were a negative control (FIG. 5A).

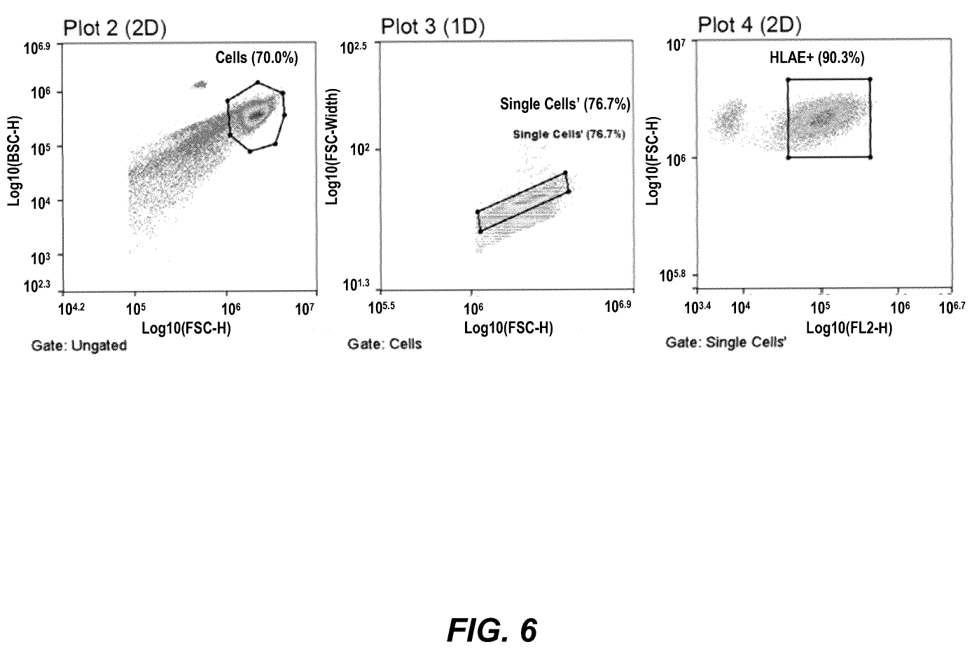

Following MACS-enrichment, the cells were single-cell sorted as described in Example 1. The anti-HLA-E-PE antibody (see Table 21) was used for FACS-sorting into 96-well plates (FIG. 6). For FACS-sorting, unedited cells served as a negative control. After sorting, the cells were expanded as described in Example 1 and when confluent, samples were split for maintenance and genomic DNA extraction.

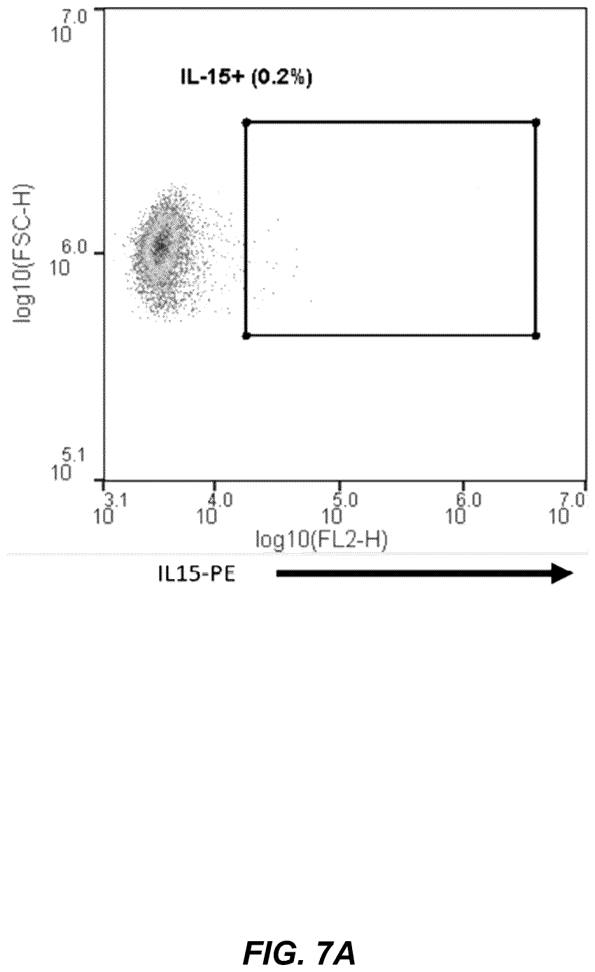

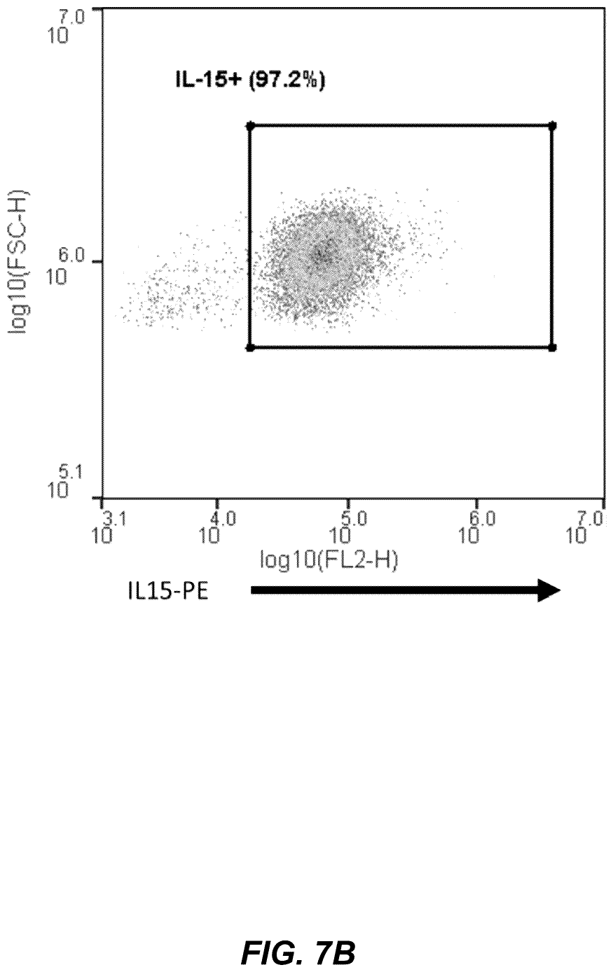

The single cell derived clones demonstrated IL15-PE expression post expansion confirming fidelity of the edit. The IL-15-PE expression in a clone named “Clone 3” (FIG. 7B) and WT iPSC control (FIG. 7A) was determined.

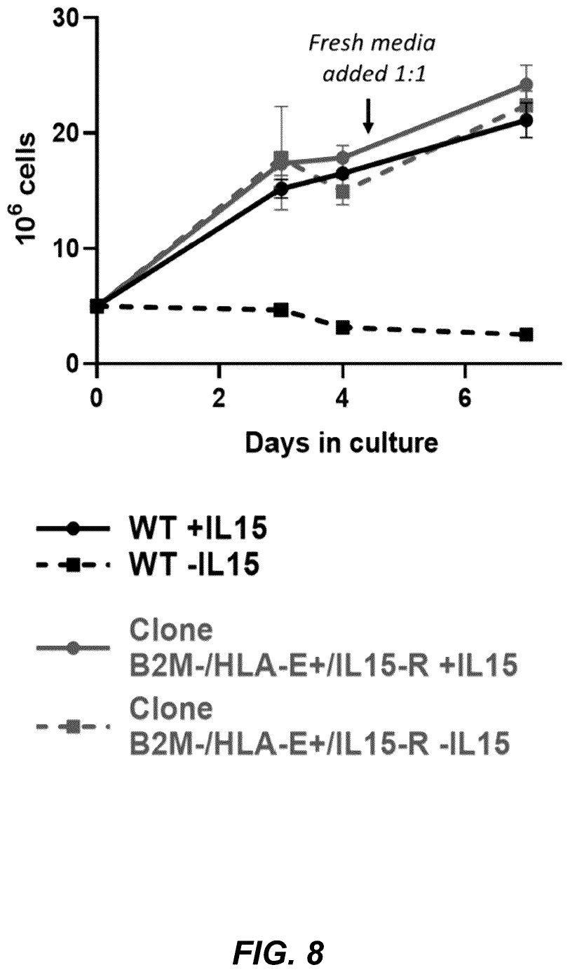

Clone 3, an hiPSC gene edited clone containing the edits IL15/IL15Rα-P2A-HLA-E trimer knock-in, B2M null, was differentiated to iNK cells using Protocol 1.5, as described in Example 3, using PBS spinner vessels. Day 20 iNK cells differentiated from WT or Clone 3 (IL15/IL15Rα-P2A-HLA-E trimer knock-in, B2M null hPSC) were plated at 5×10⁶ cells/well and grown with or without exogenous IL15 (20 ng/ml). In addition, all cells were administered SCF (20 ng/ml), Flt3L (15 ng/mL), IL-7 (20 ng/ml) on day 0 and day 4. Clone 3 (IL15/IL15Rα-P2A-HLA-E trimer knock-in, B2M null hPSC) derived iNK expanded similarly in the presence or absence of exogenous IL15 in the culture media. FIG. 8 shows that the clone 3 cells persisted and expanded in the absence of exogenous IL15 while the WT iNK cell number declined in the absence of exogenous IL15.

The cytotoxicity of the day 36 Clone 3 derived iNK cells towards K562 cells was determined using a 24-hour killing assay. K562-GFP cells (50,000 cells per vial) were incubated with iNK effector cell lines at different ratios as indicated for 24 hours. After incubation, the cells were spun, and resuspended in 175 μl media containing SyTox Blue at a 1:1000 concentration. 25 μL of countbright beads per well were added. The plate was read using the Flow cytometer 100 μL volume per well was collected for analysis. GFP-positive, SyTox Blue-negative target cells (live cancer cells) and countbright beads were selected and measured absolute events count. Total live cells were calculated as follows:

[Total Cells=((No of live cells)/(Bead count for that sample))/(Bead count per50 μL/2).

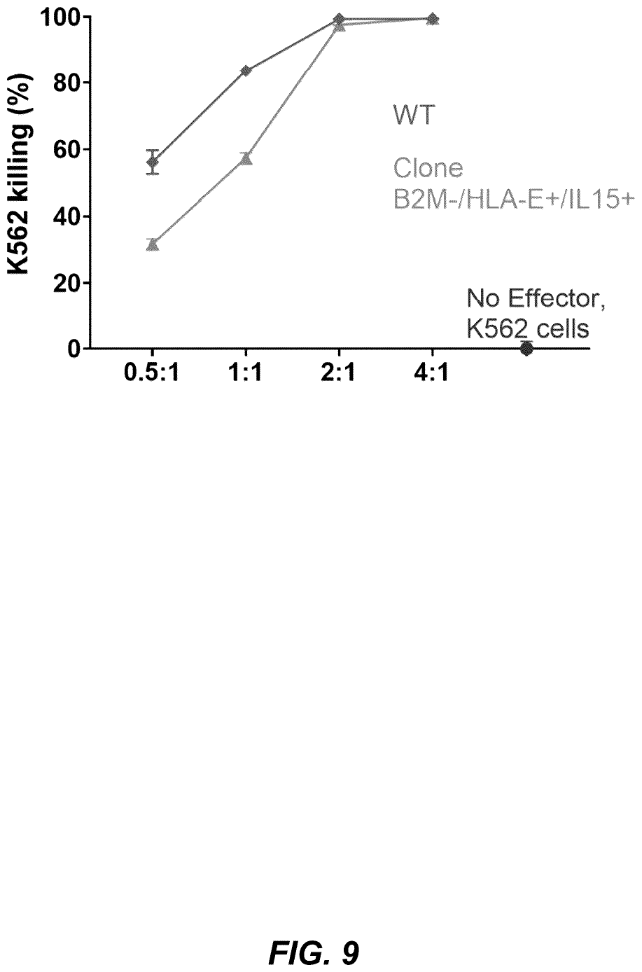

The % of cell lysis was calculated using following formula: % Cell lysis=(1−((Total Number of target Cells in Test Sample)/(Total Number of Target Cells in Control Sample))×100. The WT and edited lines displayed effective cytotoxicity against K562 (FIG. 9).

### Example 9

Generation of IL15/IL15Rα-P2A-HLA-E Trimer Knock-In, BCMA CAR Knock-In, CIITA Null, B2M Null, ADAM17 Null Human Pluripotent Stem Cells (hPSCs)

### Design of ADAM17 KO

The ADAM17-T2 gRNA (target sequence shown in Table 19) was used to knock-out the ADAM17 protein by causing a frameshift mutation in the ADAM17 gene exon 1. BCMA CAR and IL15/IL15Rα-P2A-HLA-E trimer enriched hiPSCs were generated as described in Examples 6 and 7. Electroporation was carried out in these enriched hiPSC cells using the Neon Electroporator with the RNP mixture of Cas9 protein (Biomay) and guide RNA (IDT) at a molar ratio of 5:1 (gRNA: Cas9) with absolute values of 125 μmol Cas9 and 625 μmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). This mixture was then combined with the cells to a total volume of ~115 μL using R-buffer. This mixture was then electroporated with 1 pulse for 20 ms at 1500 V followed by 1 pulse for 100 ms at 500 V. Following electroporation, the cells were pipetted out into a 6 well plate filled with STEMFLEX™ media with REVITACELL™ Supplement (100×) and laminin 511. Cells were cultured in a normoxia incubator (37° C., 8% CO₂).

Three to five days post electroporation, the cells were single-cell sorted as described in Example 1. The anti-BCMA CAR antibody (see Table 21) was used for FACS-sorting into 96-well plates. For FACS-sorting, unedited cells served as a negative control. After sorting, the cells were expanded as described in Example 1 and when confluent, samples were split for maintenance and genomic DNA extraction.

PCR for the genotyping of the edited clones (IL15/IL15Rα-P2A-HLA-E trimer knock-in, BCMA CAR knock-in, CIITA Null, B2M Null, ADAM17 Null Human Pluripotent Stem Cells (hPSCs)) was performed and the resulting amplified DNA was assessed for cutting efficiency by TIDE analysis.

For determining indels in the target B2M sequence, PCR for relevant regions was performed using Platinum Taq Supermix (Invitrogen, cat #125320176 and Cat #11495017). The sequences of the PCR primers are presented in Table 22; and the cycling conditions provided in Table 23.

TABLE 22
B2M INDEL PRIMERS

| | | | SEQ |
|---|---|---|---|
| Name | Type | Sequence (5′-3′) | ID NO: |
| B2MF2 | Forward | CAGACAGCAAACTCACCCAG | 56 |
| B2MR2 | Reverse | AAACTTTGTCCCGACCCTCC | 57 |

TABLE 23
B2M INDEL PCR CYCLING PARAMETERS

| Step | Temperature | Time | | Cycles |
|---|---|---|---|---|
| Denaturation | 94° C. | 2 | min | 1 |
| Denaturation | 94° C. | 15 | sec | 30 |
| Annealing | 56° C. | 30 | sec | |
| Extension | 68° C. | 45 | sec | |
| Elongation | 68° C. | 5 | min | 1 |

| | | |
|---|---|---|
| Hold | 4° C. | hold |

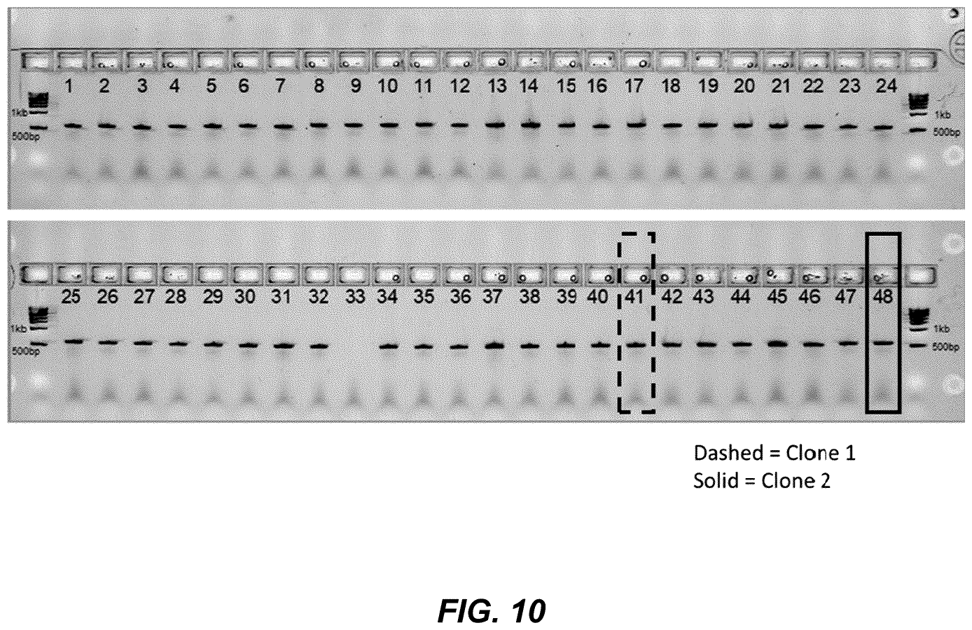

FIG. 10 shows the B2M indel results for various edited clones. The presence of a 573 bp band indicated a WT genotype which would be found in clones that are unedited or are heterozygous for the KI construct, as homozygous clones will not have a band. For determining B2M zygosity, PCR for relevant regions was performed using Platinum Taq Supermix (Invitrogen, cat #125320176 and Cat #11495017). The sequence of the PCR primers are presented in Table 24; and the cycling conditions provided in Table 25.

TABLE 24
B2M ZYGOSITY PRIMERS

| | | | SEQ |
|---|---|---|---|
| Name | Type | Sequence (5′-3′) | ID NO: |
| B2M-geno-F1 | forward | AAAAGATCTGTGGACTCCAC | 58 |
| | | CACCACGAAATGGCGGCACC | |
| | | TTATTTATGGTC | |
| B2M-geno-R1 | reverse | GCTCTGGAGAATCTCACGCA | 59 |
| | | GAAGGCAGGCGTTTTTCTTA | |
| | | AAAAAAAATGCACGAATTA | |

TABLE 25
B2M ZYGOSITY PCR CYCLING PARAMETERS

| | | | |
|---|---|---|---|
| Step | Temperature | Time | Cycles |

| Denaturation | 94° | C. | 2 | min | 1 |
|---|---|---|---|---|---|
| Denaturation | 98° | C. | 10 | sec | 30 |
| Annealing | 65° | C. | 30 | sec | |
| Extension | 68° | C. | 6 min 30 sec | | |
| Elongation | 68° | C. | 5 | min | 1 |

| | | | |
|---|---|---|---|
| Hold | 4° | C. | hold |

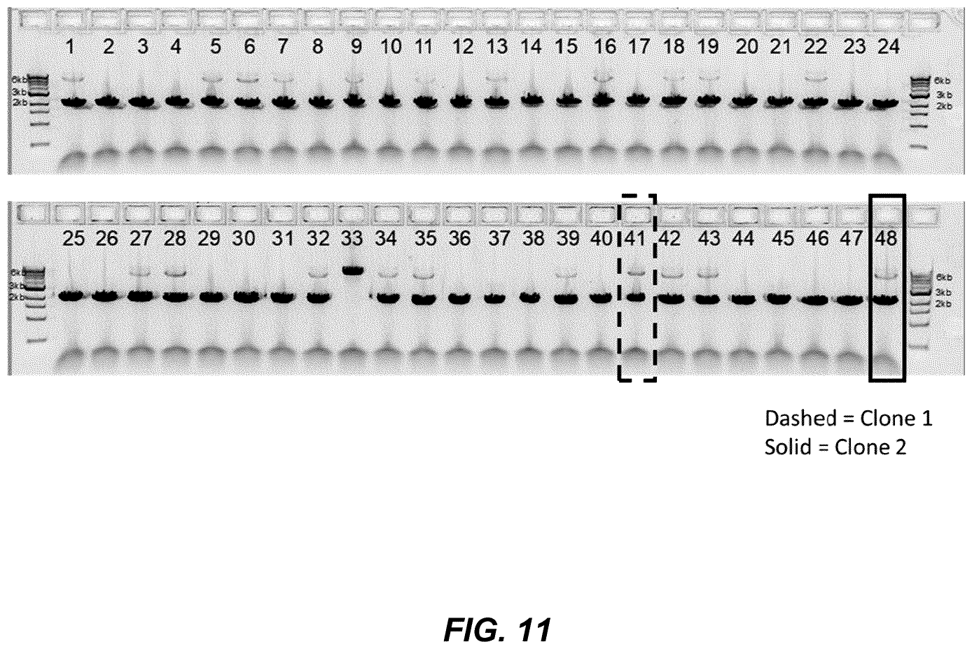

FIG. 11 shows the B2M zygosity results for various edited clones. The presence of a ~2.5 kb band indicated a WT genotype while the presence of a 6.6 kb band indicated successful integration of the KI construct into the B2M gene locus. Unedited clones would only have the WT band, clone heterozygous for the KI would have both bands, and homozygous clones would only have the KI band. The resulting amplicons were submitted for PCR cleanup and Sanger sequencing. Sanger sequencing results were input into Tsunami software along with the guide sequence. The resulting DNA sequences of the target B2M region were aligned in Snapgene software to determine indel identity and homo- or heterozygosity. For determining IL15/IL15Rα-P2A-HLA-E trimer knock-in genotyping in the target B2M sequence, PCR for relevant regions was performed using Platinum Taq Supermix (Invitrogen, cat #125320176 and Cat #11495017). The sequence of the PCR primers are presented in Table 26; and the cycling conditions provided in Table 27.

TABLE 26
B2M KI PRIMERS

| | | | SEQ |
|---|---|---|---|
| Name | Type | Sequence (5′-3′) | ID NO: |
| Poly-A-F | forward | AGGATTGGGAAGACAATAGC | 60 |
| | | AGGCATGCTGGGGATGCGGT | |
| | | GG | |
| B2M-geno-R1 | reverse | GCTCTGGAGAATCTCACGCA | 61 |
| | | GAAGGCAGGCGTTTTTCTTA | |
| | | AAAAAAAATGCACGAATTA | |

TABLE 27
B2M KI PCR CYCLING PARAMETERS

| | | | |
|---|---|---|---|
| Step | Temperature | Time | Cycles |

| Denaturation | 98° | C. | 30 | sec | 1 |
|---|---|---|---|---|---|
| Denaturation | 98° | C. | 10 | sec | 30 |
| Annealing | 65° | C. | 30 | sec | |
| Extension | 72° | C. | 1 min 30 sec | | |
| Elongation | 72° | C. | 5 | min | 1 |

| | | |
|---|---|---|
| Hold | 4 | hold |

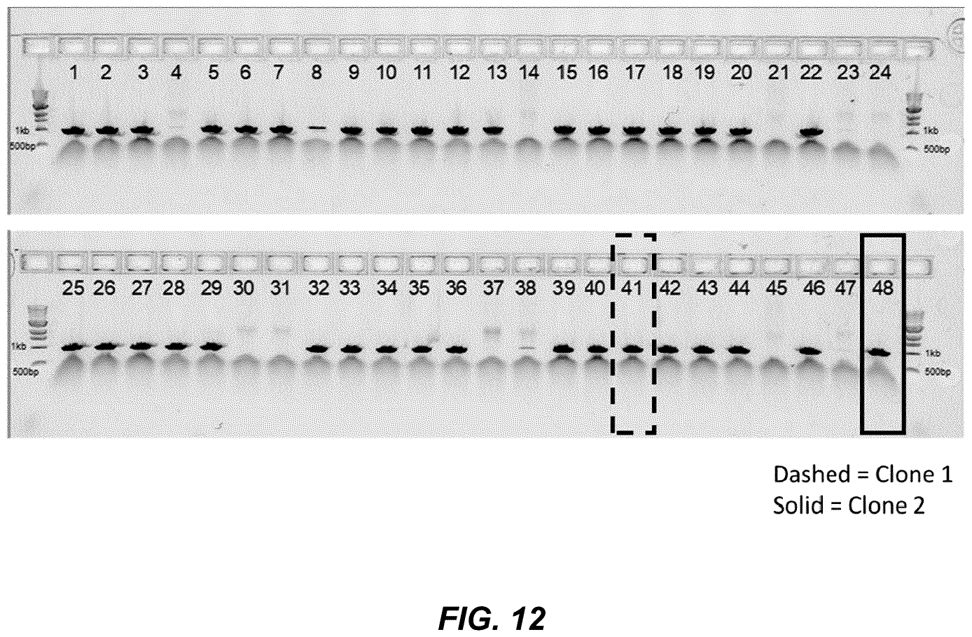

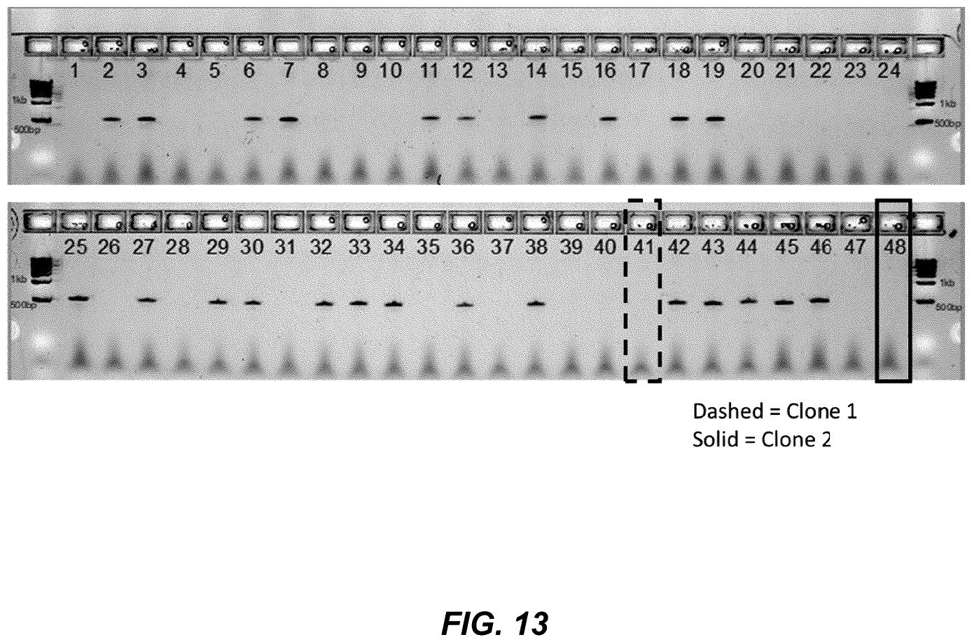

FIG. 12 shows the B2M KI genotyping results for various edited clones. The presence of a 1.1 kb band indicated successful integration of the KI construct into the B2M gene locus, while the absence of a band indicated a WT genotype. For determining indels in the target CIITA sequence, PCR for relevant regions was performed using Platinum Taq Supermix (Invitrogen, cat #125320176 and Cat #11495017). The sequence of the PCR primers are presented in Table 16; and the cycling conditions provided in Table 17. FIG. 13 shows the CIITA indel results for various edited clones. The presence of a 557 bp band indicated a WT genotype which would be found in clones that are unedited or are heterozygous for the KI construct, as homozygous clones will not have a band.

For determining CIITA zygosity, PCR for relevant regions was performed using Platinum Taq Supermix (Invitrogen, cat #125320176 and Cat #11495017). The sequence of the PCR primers are presented in Table 28; and the cycling conditions provided in Table 29.

TABLE 28
CIITA ZYGOSITY PRIMERS

| | | | SEQ |
|---|---|---|---|
| Name | Type | Sequence (5′-3′) | ID NO: |
| CIITA-OUT-F | forward | GCCCCACCCCTCCTACTTTA | 62 |
| | | TGTCTCCATGGATTTGCCTG | |
| | | TTTTGGTCATTTCA | |
| CIITA-OUT-R | reverse | CTCTAATGCAAACTTGGGTA | 63 |
| | | GGTCGTTTCACCTCTCTAAA | |
| | | CCTCAATTTCCTCATTTG | |

TABLE 29
CIITA ZYGOSITY PCR CYCLING PARAMETERS

| | | | |
|---|---|---|---|
| Step | Temperature | Time | Cycles |

| Denaturation | 94° | C. | 2 | min | 1 |
|---|---|---|---|---|---|
| Denaturation | 98° | C. | 10 | sec | 30 |
| Annealing | 65° | C. | 30 | sec | |
| Extension | 68° | C. | 5 min 30 sec | | |
| Elongation | 68° | C. | 5 | min | 1 |

| | | |
|---|---|---|
| Hold | 4 | hold |

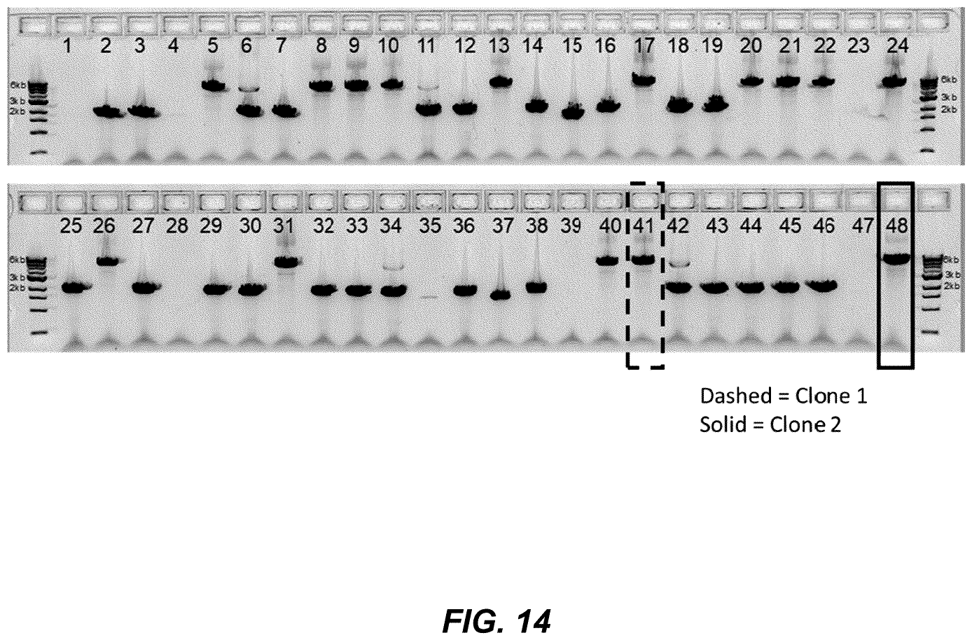

FIG. 14 shows the CIITA zygosity results for various edited clones. The presence of a ~2.5 kb band indicated a WT genotype while the presence of a 5.6 kb band indicated successful integration of the KI construct into the CIITA gene locus. Unedited clones would only have the WT band, clones heterozygous for the KI would have both bands, and homozygous clones would only have the KI band. The resulting amplicons were submitted for PCR cleanup and Sanger sequencing. Sanger sequencing results were input into Tsunami software along with the guide sequence. The resulting DNA sequences of the target CIITA region were aligned in Snapgene software to determine indel identity and homo- or heterozygosity.

For determining BCMA CAR knock-in genotyping in the target CIITA sequence, PCR for relevant regions was performed using Platinum Taq Supermix (Invitrogen, cat #125320176 and Cat #11495017). The sequence of the PCR primers are presented in Table 30; and the cycling conditions provided in Table 31.

TABLE 30
CIITA KI PRIMER

| | | | SEQ |
|---|---|---|---|
| Name | Type | Sequence (5′-3′) | ID NO: |
| CD3Z-seq-F1 | forward | GAGTGAAGTTTTCCCGAAG | 64 |
| | | CGCAGACGCTCCGGCATAT | |
| | | CAGCAAGGACAG | |
| CIITA-OUT-R | reverse | CTCTAATGCAAACTTGGGT | 65 |
| | | AGGTCGTTTCACCTCTCTA | |
| | | AACCTCAATTTCCTCATTT | |
| | | G | |

TABLE 31
CIITA KI PCR CYCLING PARAMETERS

| | | | |
|---|---|---|---|
| Step | Temperature | Time | Cycles |

| Denaturation | 98° | C. | 30 | sec | 1 |
|---|---|---|---|---|---|
| Denaturation | 98° | C. | 10 | sec | 30 |
| Annealing | 65° | C. | 30 | sec | |
| Extension | 72° | C. | 1 min 30 sec | | |
| Elongation | 72° | C. | 5 | min | 1 |

| | | |
|---|---|---|
| Hold | 4 | hold |

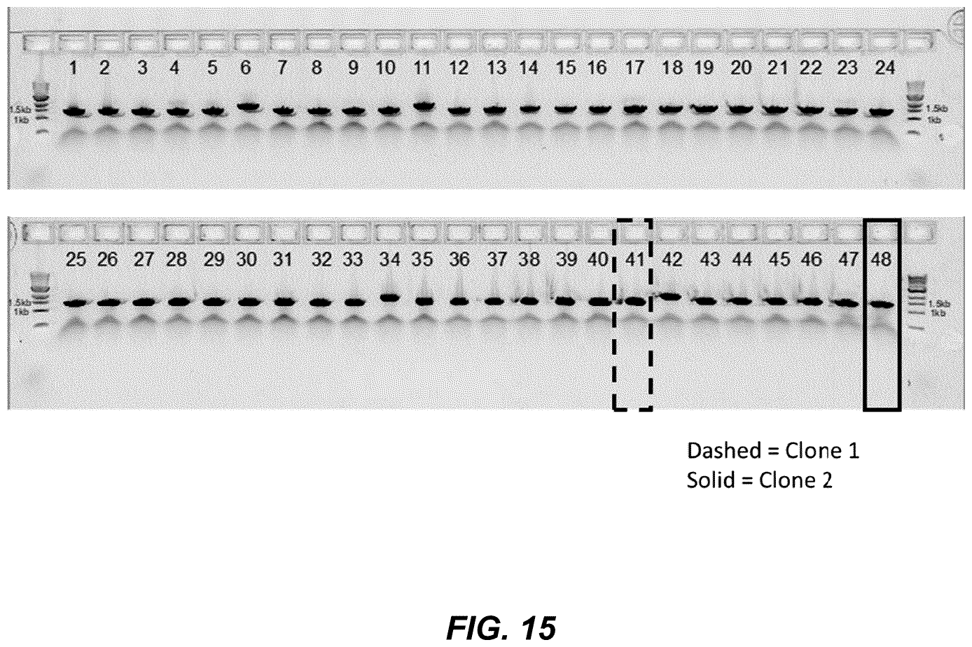

FIG. 15 shows the CIITA KI genotyping results for various edited clones. The presence of a 1.5 kb band indicated successful integration of the KI construct into the CIITA gene locus, while the absence of a band indicated a WT genotype. For determining indels in the target ADAM17 sequence, PCR for relevant regions was performed using Platinum Taq Supermix (Invitrogen, cat #125320176 and Cat #11495017). The sequence of the PCR primers are presented in Table 13; and the cycling conditions provided in Table 14. The resulting amplicons were submitted for PCR cleanup and Sanger sequencing. Sanger sequencing results were input into Tsunami software along with the guide sequence. The resulting DNA sequences of the target ADAM17 region were aligned in Snapgene software to determine indel identity and homo- or heterozygosity.

Based on the PCR and Sanger sequencing analysis of the edited clones, the clone shown in lane 41 in FIG. 10-FIG. 15 was chosen as “clone 1” and the clone shown in lane 48 was chosen as “clone 2,” which were shown to have the BCMA CAR KI and the IL15/ILRa-P2A-HLA-E KI, while the sequencing data confirmed that B2M, CIITA, and ADAM17 were completely knocked-out. Clone 1 was heterozygous for the B2M KI and had an indel of +1T in the B2M WT band (Table 32). Clone 1 was homozygous for the CIITA KI and contained a homozygous +1G indel in the ADAM17 WT band (Table 33).

TABLE 32
KI GENOTYPES OF IL15/IL15Rα-P2A-HLA-E TRIMER
KNOCK-IN, BCMA CAR KNOCK-IN, CIITA NULL, B2M NULL,
ADAM17 NULL HUMAN PLURIPOTENT STEM CELLS CLONES

| | IL-15/IR-15 fusion- | BCMA CAR |
|---|---|---|
| Clone | P2A-HLA-E into B2M | into CIITA |
| 1 | Heterozygous KI | Homozygous KI |
| 2 | Heterozygous KI | Homozygous KI |

TABLE 33
KO GENOTYPES OF IL15/IL15Rα-P2A-HLA-E TRIMER
KNOCK-IN, BCMA CAR KNOCK-IN, CIITA NULL, B2M NULL,
ADAM17 NULL HUMAN PLURIPOTENT STEM CELLS CLONES

| Clone | B2M indel | CIITA indel | ADAM17 indel |
|---|---|---|---|
| 1 | KI/+1 T | KI/KI | +1 G/+1 G |
| 2 | KI/+1 T | KI/KI | −20/large insertion |

Confirmation of KI gene expression and KO status at the hiPSC stage. To detect the BCMA CAR, HLA-E, and IL15 surface expression, fluorescent antibodies were used (see Table 21). Undifferentiated clone 1, the hiPSC clone containing all the edits (IL15/IL15Rα-P2A-HLA-E trimer knock-in, BCMA CAR knock-in, CIITA Null, B2M Null, ADAM17 Null) was assessed by flow cytometry with unedited WT cells as a negative control (Table 34). The gene edited clone 1 showed >99% BCMA CAR expression, >99% HLA-E expression, and >99% IL15 expression. To confirm KO status, fluorescent antibodies for HLA-ABC were used (see Table 21) with unedited WT iNK cells as a negative control (Table 34).

TABLE 34
FLOW CYTOMETRY RESULTS

| | WT iPSC | Clone 1 iPSC |
|---|---|---|
| CAR⁺ | 1.06% | 99.7% |
| HLA-E⁺ | 0.22% | 100% |
| IL-15⁺ | 0.03% | 99.2% |
| HLA-A, B, C (MHC-I)⁻ | 97.3% | 0.74% |

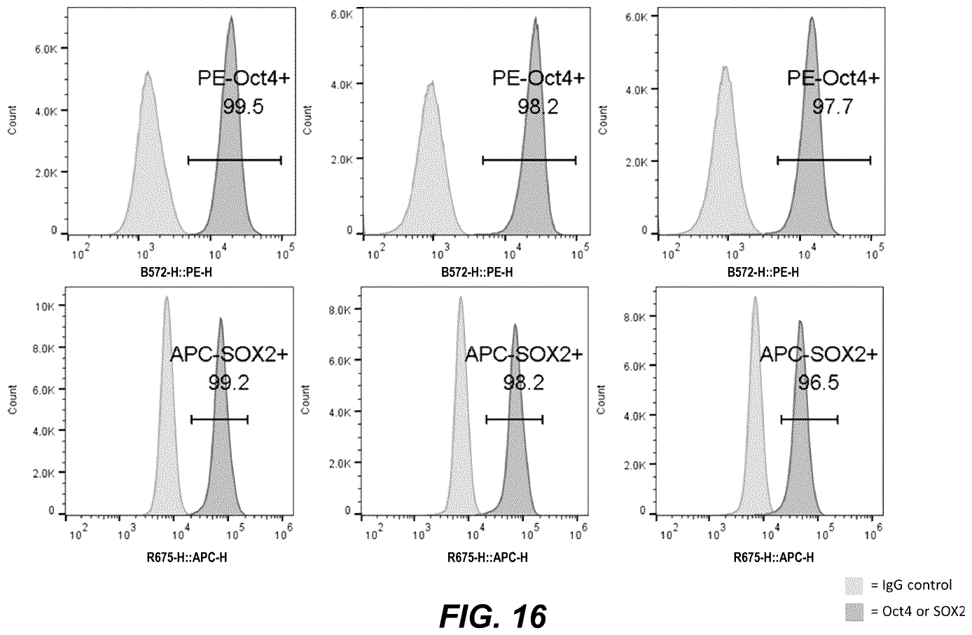

Confirmation of hiPSC pluripotency after genome editing. To detect the Oct4 and Sox2 intracellular expression fluorescent antibodies were used. iPS cells: WT and undifferentiated clones 1 and 2, containing all the edits (IL15/IL15Rα-P2A-HLA-E trimer knock-in, BCMA CAR knock-in, CIITA null, B2M null, ADAM17 null) were assessed by flow cytometry. IgG-labeled cells served as a negative control (FIG. 16). Oct4 expression was 99.5% in WT, 98.2% in the gene edited clone 1 and 97.7% in the gene edited clone 2 (FIG. 16). There were the following percentages of Sox2-positive cells in iPSC populations: WT iPSC had >99%, edited clones 1 and 2 had >98 and >96% positive correspondently (FIG. 16). Edited clones retained high level of pluripotency.

### Example 10

Differentiation and Characterization of IL15/IL15Rα-P2A-HLA-E Trimer Knock-In, BCMA CAR Knock-In, CIITA Null, B2M Null, ADAM17 Null hPSC

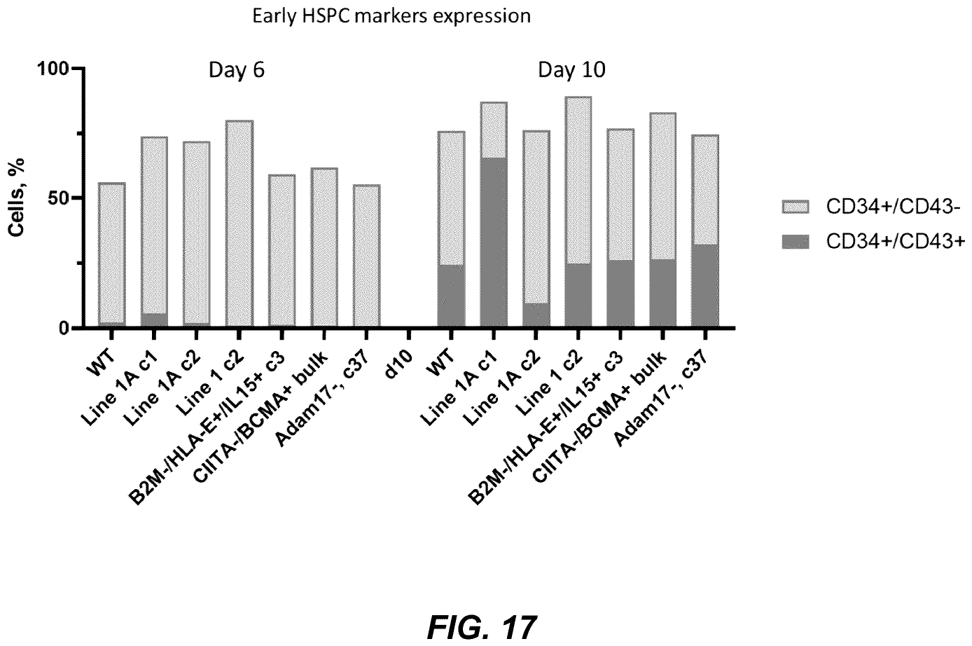

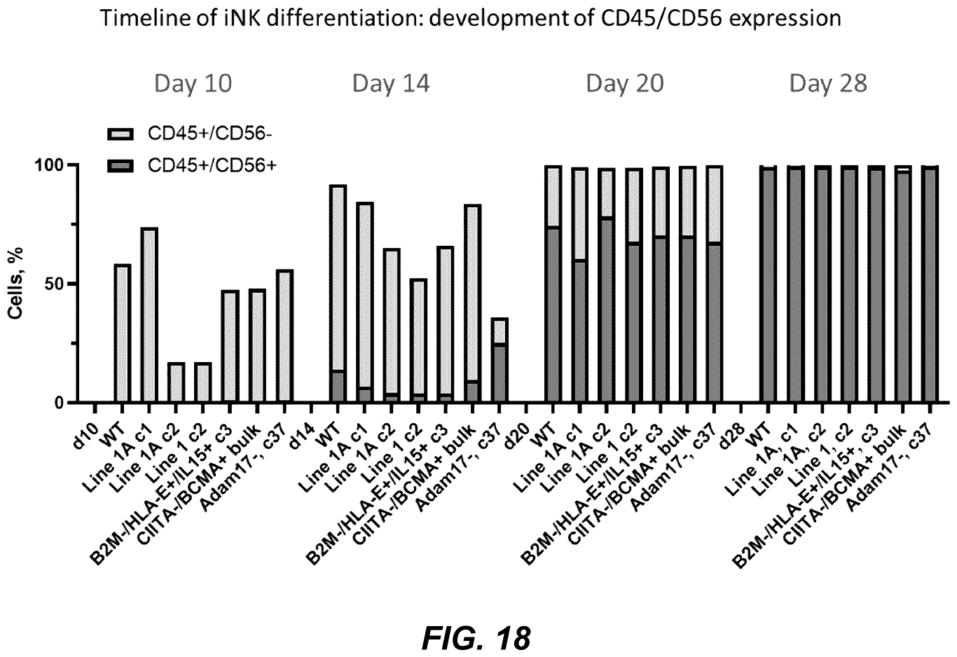

WT, Clones 1 and 2 (“Line1A c1 and c2”; hiPSC gene edited clones containing the edits IL15/IL15Rα-P2A-HLA-E trimer knock-in, BCMA CAR knock-in, CIITA null, B2M null, ADAM17 null), Clone 3 (“B2M-/HLA-E+/IL15+c3”); hiPSC gene edited clone containing the edits IL15/IL15Rα-P2A-HLA-E trimer knock-in, B2M null), Line 1 clone 2 (“Line 1 c2”; hiPSC gene edited clone containing the edits IL15/IL15Rα-P2A-HLA-E trimer knock-in, BCMA CAR knock-in, CIITA null, B2M null), a CIITA-/BCMA CAR+ bulk population, and a ADAM17 KO clone 37 (“Adam17-, c37”) were differentiated to iNK cells using Protocol 1, as described in Example 2. Flow cytometry of differentiated cells at Days 6 and 10 showed that all of the edited clones and bulk populations, including both edited iPSC clones 1 and 2, differentiated efficiently to HPSC (CD34+/CD43-) cell population as compared with WT (FIG. 17). Edited iPSC clone 1 expressed CD43 earlier but that did not influence its overall differentiation into iNK and cytotoxicity. Throughout the differentiation process, cells were analyzed for CD45 and CD56 expression by flow cytometry (FIG. 18), showing efficient differentiation for all of the edited clones which was comparable to WT. By day 28, >99% of cells are CD56⁺.

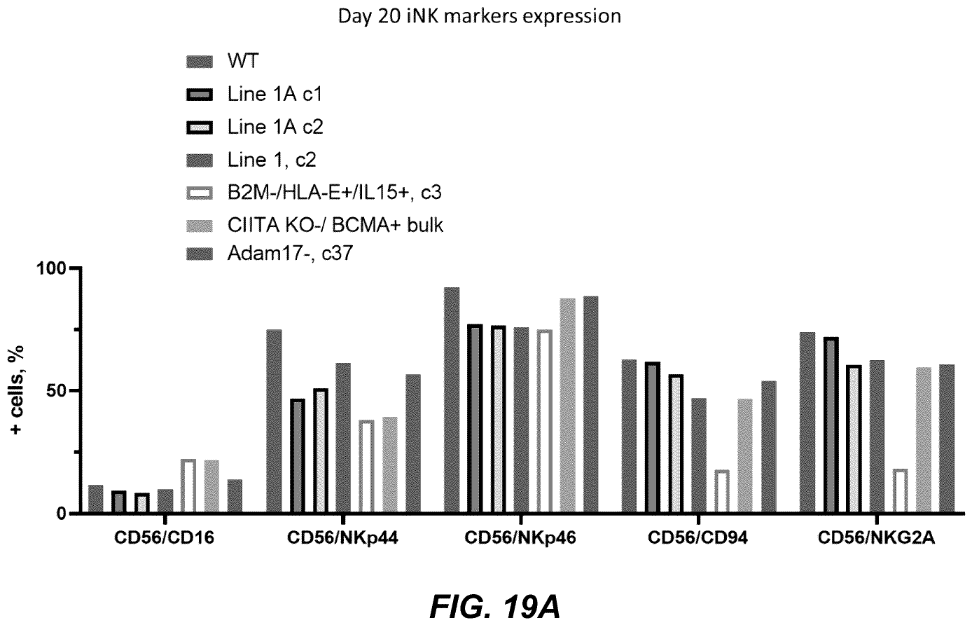

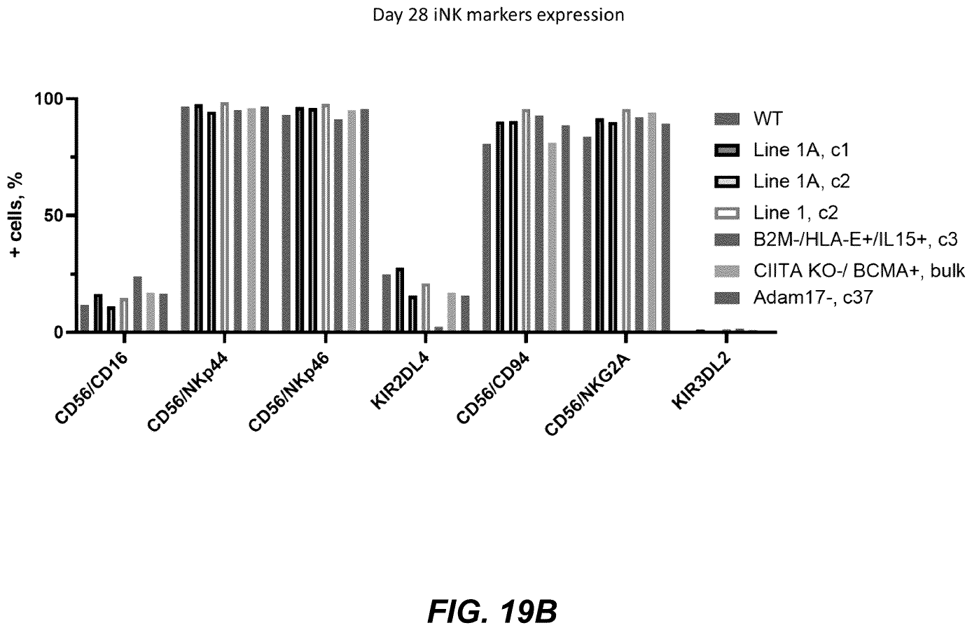

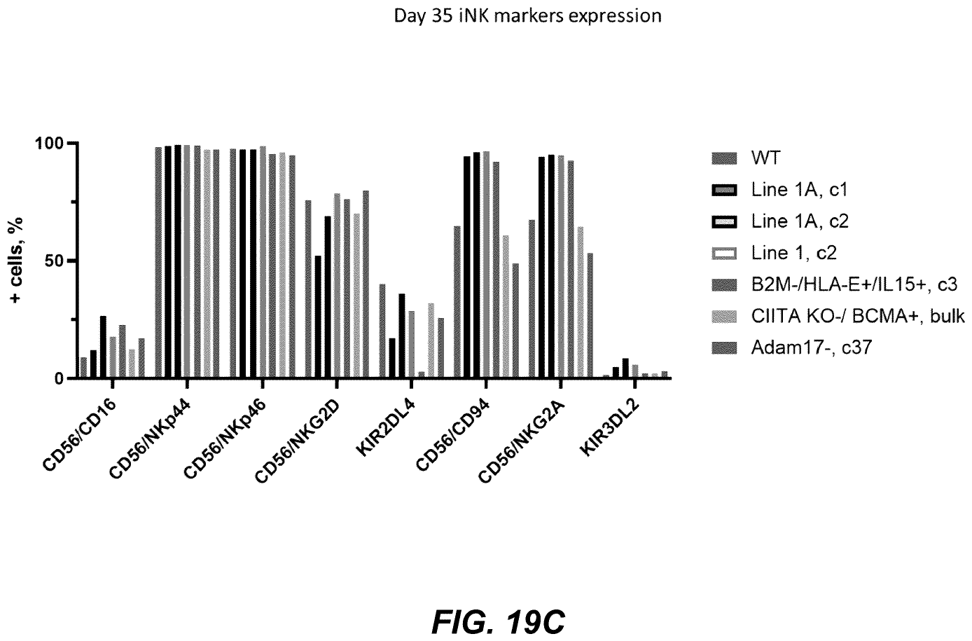

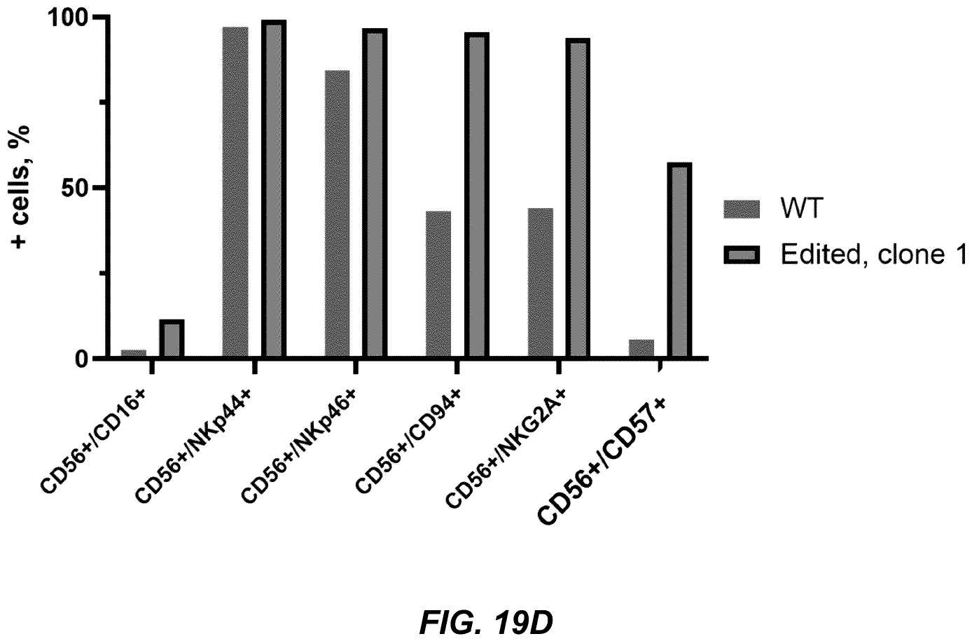

Flow cytometry was performed on digested cells aggregates on days 6, 10, and 14; and on single cells on days 20 (FIG. 19A), 28 (FIG. 19B), 35 (FIG. 19C), and 42 (FIG. 19D). Live cells were collected, washed with 1% BSA in PBS, and incubated with appropriate antibody cocktails in 5% BSA in PBS for 30 min on ice. The cells were washed and resuspended in 1% BSA in PBS containing 1:1000 SyTOX Blue cells viability dye followed by loading the plate on the Flow cytometer for analysis (see Table 35 for antibodies used). The differentiated Line 1A, clone 1 and 2, as well as edited IL15/IL15Rα-P2A-HLA-E trimer knock-in into B2M null, clone 3 iNK cells expressed a majority of maturation markers. On day 20 of differentiation all three edited lines displayed NK markers expression that was somewhat lower than WT. However the same markers were expressed at comparable or even higher than WT levels only a week later (at day 28).

TABLE 35
ANTIBODIES

| antigen | fluorophore | company | catalog # | Dilution |
|---|---|---|---|---|
| CD16 | PE-Cy7 | BioLegend | 360708 | 1:50 |
| CD235a/Glycophorin A | APC | BioLegend | 349114 | 1:10 |
| CD34 | FITC | Miltenyi | 130-113-178 | 1:25 |
| CD34 | PE | BD | 555822 | 1:10 |
| CD43 | BB515 | BD | 564542 | 1:500 |
| CD45 | PE-Cy7 | BD | 557748 | 1:100 |
| CD45 | BB515 | BD | 564585 | 1:100 |
| CD56 | PE | Miltenyi | 130-113-307 | 1:500 |
| CD56 | BB515 | BD | 564488 | 1:25 |
| CD56/NCAM1 | APC | BD | 555518 | 1:10 |
| CD57 | PE-Cy7 | BioLegend | 359624 | 1:10 |
| CD94/KLRD1 | APC | Miltenyi | 130-098-976 | 1:5 |
| CD95/Fas1 | FITC | BD | 555673 | 1:10 |
| HLA-ABC | FITC | eBioscience | 11-9983-42 | 1:10 |
| HLA-DR, DP, DQ | 647 | BioLegend | 361703 | 1:10 |
| HLA-E | APC | BioLegened | 342605 | 1:10 |
| hTACE/ADAM17 | PE | R&D | FAB9301P | 1:10 |
| IL-15 | APC | Invitrogen | MA5-23627 | 1:10 |
| IL-15 | PE | Invitrogen | MA5-23561 | 1:10 |
| IL-15 | FITC | Invitrogen | MA5-23664 | 1:10 |
| KIR2DL4/CD158d | APC | Miltenyi | 130-112-466 | 1:25 |
| KIR3DL2/CD158e/k | PE-Vio770 | Miltenyi | 130-116-180 | 1:100 |
| NKG2A/CD159a | APC | Miltenyi | 130-113-563 | 1:5 |
| NKG2D | BB515 | BD | 564566 | 1:2.5 |
| NKp44/CD336 | PE | BD | 558563 | 1:5 |
| NKp46/CD335 | PE-Cy7 | BD | 562101 | 1:5 |
| Oct3/4 | PE | BD Bioscience | 560186 | 1:10 |
| PD-1/CD279 | APC | BioLegend | 621610 | 1:10 |
| PD-L1/CD274 | PE-Cy7 | BD | 558017 | 1:10 |
| SOX2 | Alexa 647 | BD Bioscience | 562139 | 1:10 |
| Perforin, Clone delta G9, | PE | Miltenyi | 130-123-726 | 1:25 |
| Granzyme B Clone REA226 | APC | Miltenyi | 130-120-773 | 1:25 |

Confirmation of KI gene expression and KO status of edited cells differentiated to the INK stage. Using these differentiated Line 1A clone 1 cells, flow cytometry was repeated to assess KI gene expression and KO status. To detect the BCMA CAR, HLA-E, and IL15 surface expression, fluorescent antibodies were used (see Table 21) with unedited WT iNK cells as a negative control (Table 36). The Line 1A clone 1-derived iNK cells showed >99% BCMA CAR expression, >90% HLA-E expression, and >99% IL15 expression. To confirm KO status, fluorescent antibodies for HLA-ABC were used (see Table 21) with unedited WT iNK cells as a negative control (Table 36).

TABLE 36
FLOW CYTOMETRY RESULTS

| | WT iNK | Clone 1 iNK |
|---|---|---|
| CAR⁺ | 0.75% | 99.9% |
| HLA-E⁺ | 5.65% | 91% |
| IL-15⁺ | 0.33% | 99.1% |
| HLA-A, B, C (MHC-I)⁻ | 99.8% | 0.8% |

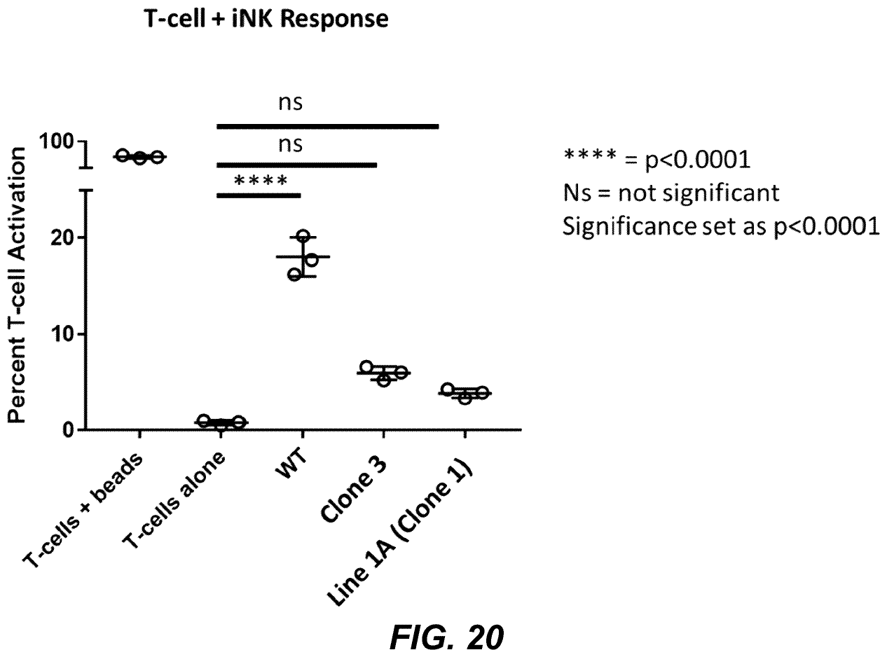

Immune phenotype of edited iNK cells. At the iNK stage, differentiated cells of clone 1 (an hiPSC gene edited clone containing all the edits (IL15/IL15Rα-P2A-HLA-E trimer knock-in, BCMA CAR knock-in, CIITA null, B2M null, ADAM17 null) of Example 9 and clone 3 (an hiPSC gene edited clone containing B2M KO (IL15/IL15Rα-P2A-HLA-E trimer knock-in, B2M null) of Example 8 and differentiated wild-type iPSC cells were co-cultured with donor derived T-cells that were labeled with CFSE. After 5 days of co-culture, the cells were analyzed by flow cytometry and the degree of CFSE loss was assessed. WT iNK cells induced a loss of CFSE signal in the T-cells, suggesting an allogeneic immune reaction had occurred. iNK cells derived from clone 1 or clone 3 did not produce CFSE loss in the T-cells, suggesting that these cells were immune-evasive (FIG. 20).

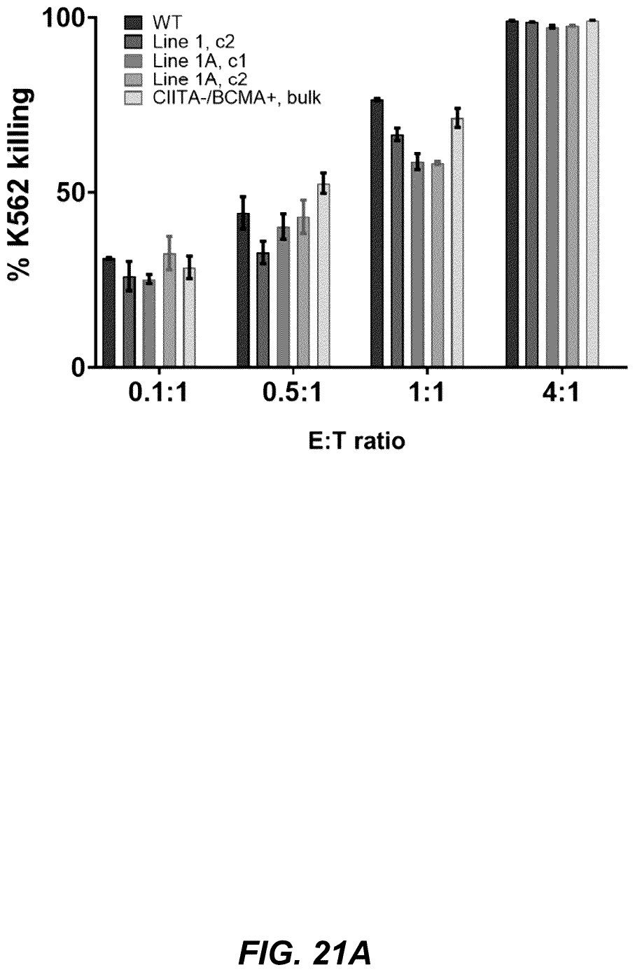

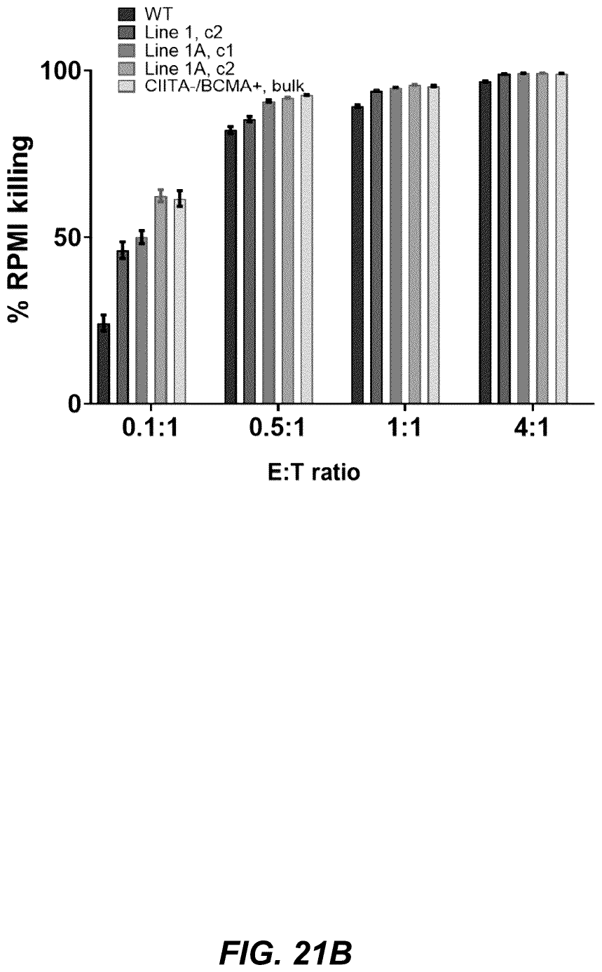

The cytotoxicity of the day 21 Line 1A clones 1 and 2, and Line 1 clone 2 (IL15/IL15Rα-P2A-HLA-E trimer knock-in, BCMA CAR knock-in, CIITA null, B2M null) cells towards K562 and RPMI cells was determined using a 24-hour killing assay, as described in Example 8. The WT and clones 1 and 2 line displayed effective cytotoxicity against K562 (FIG. 21A), while clones 1 and 2 also displayed greater cytotoxicity against BCMA⁺ expressing RPMI cancer cell line especially at lowest Effector to target cells ratio, 0.1:1 (FIG. 21B).

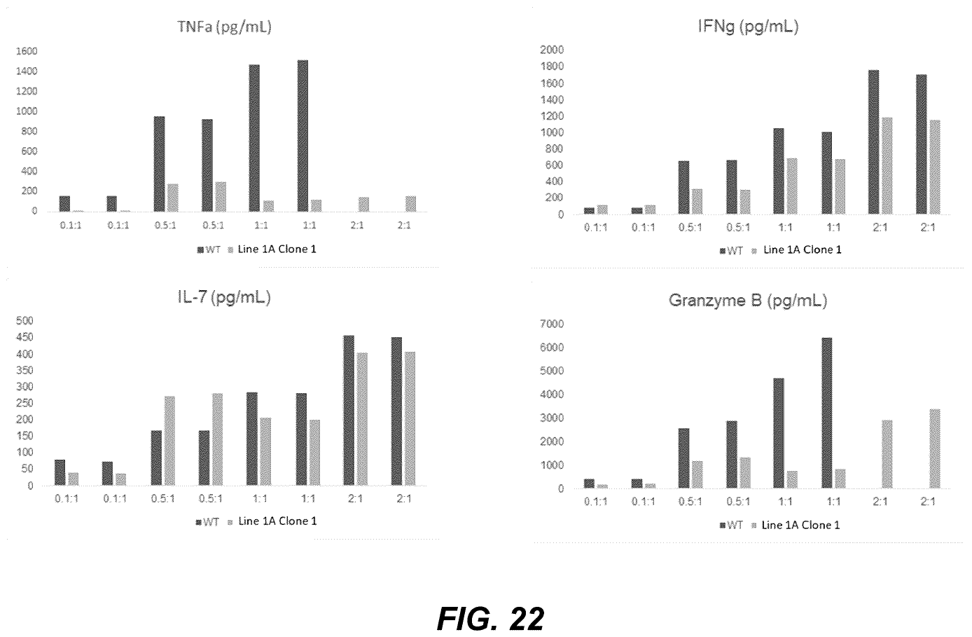

Cytokines (IFNg, TNFα,) were measured using the ProteinSimple Ella system, according to the manufacturer's instructions, with the software version v.3.5.2.20 of the Simple Plex Runner software, and Simple Plex Explorer software. Custom 8-plex Ella cartridges (32×8 Multiplex) were provided by ProteinSimple, along with dilution buffer which was used to dilute each sample (WT and Line 1A clone 1) at a 1:2 ratio prior to loading 40 μL sample per channel. As shown in FIG. 22, the IFNg levels in media correlated with an increased E:T ratio, being higher than WT in low E:T ratios (0.1:1). At higher E:T ratios, IFNg is somewhat lower in edited cells than WT, which might be the result of drastic decrease of target cells due to their efficient lysis over 24 hours. TNFα was higher in WT than in edited clone 1. This effect may be explained by lack of Adam17, a protease that cleaves TNFα.

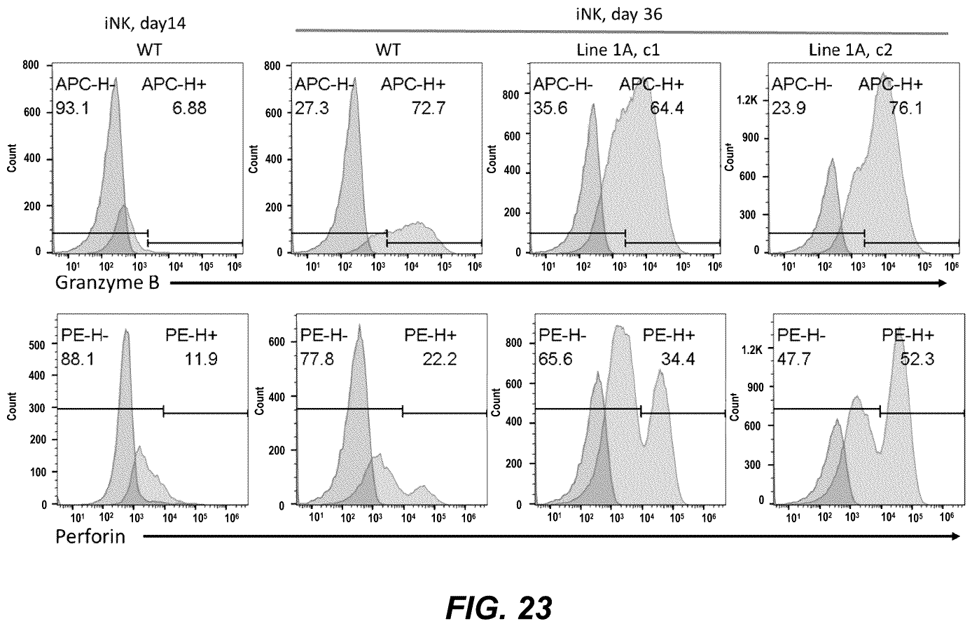

Perforin and granzyme-B expression in cells were measured by flow cytometry at day 14 and Day 36 of differentiation using commercially available antibodies. FIG. 23 shows that WT cells at day 14 of differentiation had little to no expression of perforin or granzyme-B but had higher expression at day 36. Line 1A clone 1 had similar expression patterns as WT.

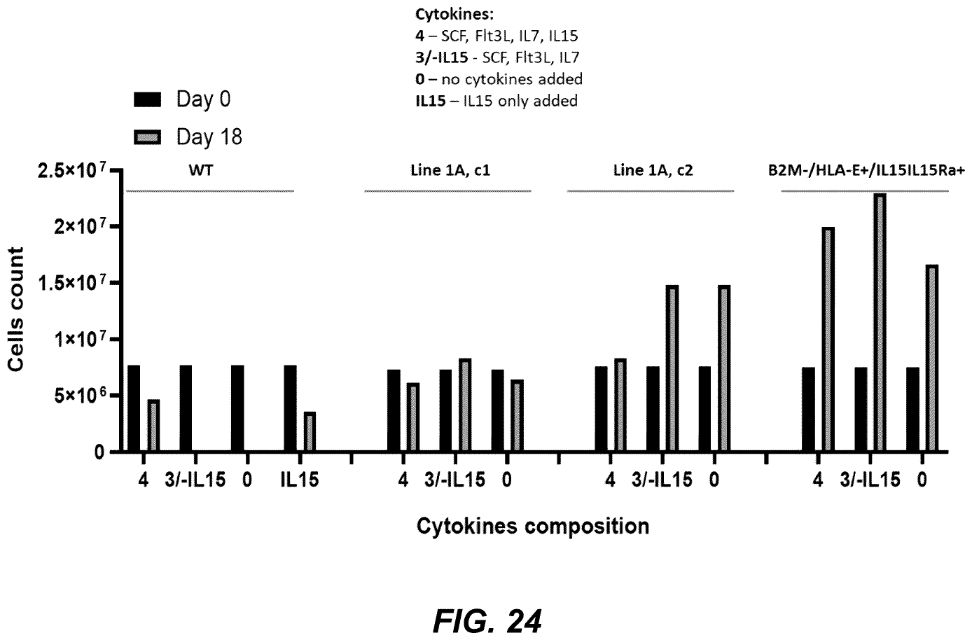

Day 20 iNKs differentiated from wild-type (WT), Line 1A clone 1 (“Line 1A, c1”), Line 1A clone 2 (“Line 1A, c2”), and Clone 3 (“B2M-/HLA-E+/IL15IL15Rα+”; IL15/IL15Rα-P2A-HLA-E trimer knock-in, B2M Null hPSC) derived iNK cells were plated at 5×10⁶ cells/well and grown with or without exogenous cytokines. Cells were administered SCF (20 ng/ml), Flt3L (15 ng/mL), IL-7 (20 ng/ml), and IL-15 (15 ng/ml) (“4”), SCF, Flt3L, and IL-7 (“3/-IL15-”), no cytokines (“0”); or only IL-15 (“IL15”) on day 0 and day 18 (FIG. 24). The edited clones persisted and expanded in the absence of exogenous IL15 while the WT iNK cell number declined in the absence of exogenous IL15.

### Example 11

Generation and Selection of FAS gRNA, CISH gRNA, and REGNASE-1 gRNA

Targeting gRNAs were designed for targeting exons 1, 2, and 3 of the FAS coding sequence, exons 1, 2, and 3 of the CISH coding sequence, exons 2 and 4 of the REGNASE-1 coding sequence. The target sequences of the gRNAs are presented in Tables 37, 38, and 39, respectively, and each gRNA comprises an RNA spacer sequence corresponding to the target DNA sequence. These gRNAs had predicted low off-target scores based on sequence homology prediction using gRNA design software.

TABLE 37
FAS TARGET SEQUENCES

| | | SEQ | |
|---|---|---|---|
| Name | Target Sequence (5′-3′) | ID NO: | PAM |
| FAS Ex1 T7 | GGATTGCTCAACAACCATGC | 35 | TGG |
| FAS Ex1 T9 | GATTGCTCAACAACCATGCT | 37 | GGG |
| FAS Ex2 T1 | GTGACTGACATCAACTCCAA | 38 | GGG |
| FAS Ex2 T2 | CACTTGGGCATTAACACTTT | 39 | TGG |
| FAS Ex2 T3 | TTGGAAGGCCTGCATCATGA | 53 | TGG |
| FAS Ex2 T7 | ACTCCAAGGGATTGGAATTG | 55 | AGG |
| FAS Ex3 T1 | CTAGGGACTGCACAGTCAAT | 80 | GGG |

TABLE 38
CISH TARGET SEQUENCES

| | | SEQ | |
|---|---|---|---|
| Name | Target Sequence (5′-3′) | ID NO: | PAM |
| CISH Ex1 T2 | TCGCCGCTGCCGCGGGGACA | 81 | TGG |
| CISH Ex1 T18 | GACATGGTCCTCTGCGTTCA | 82 | GGG |
| CISH Ex2 T1 | GTCCGCTCCACAGCCAGCAA | 83 | AGG |
| CISH Ex2 T2 | GTTCCAGGGACGGGGCCCAC | 84 | AGG |
| CISH Ex3 T1 | TCGGGCCTCGCTGGCCGTAA | 85 | TGG |
| CISH Ex3 T2 | CGTACTAAGAACGTGCCTTC | 86 | TGG |
| CISH Ex3 T3 | GGGTTCCATTACGGCCAGCG | 87 | AGG |
| CISH Ex3 T5 | CAGGTGTTGTCGGGCCTCGC | 88 | TGG |
| CISH Ex3 T6 | TACTCAATGCGTACATTGGT | 89 | GGG |
| CISH Ex3 T9 | AAGGCTGACCACATCCGGAA | 90 | AGG |
| CISH Ex3 T11 | TACATTGGTGGGGCCACGAG | 91 | TGG |
| CISH Ex3 T14 | CTGTCAGTGAAAACCACTCG | 92 | TGG |

TABLE 39
REGNASE-1 TARGET SEQUENCES

| | Target | SEQ | |
|---|---|---|---|
| Name | Sequence (5′-3′) | ID NO: | PAM |
| REGNASE-1 Ex2 T1 | GGTCATCGATGGGAGCAACG | 93 | TGG |
| REGNASE-1 Ex2 T2 | CACCACCCCGCGGGACTAGA | 94 | GGG |
| ZC3H12A_Segment | GGTCTGGCGCTCCCGCTCGG | 95 | TGG |
| 2 T3 | | | |
| REGNASE-1 Ex2 T4 | CCACCACCCCGCGGGACTAG | 96 | AGG |
| REGNASE-1 Ex2 T5 | TTAGGGGTGCCACCACCCCG | 97 | CGG |
| REGNASE-1 Ex4 T1 | TTCACACCATCACGACGCGT | 98 | GGG |
| ZC3H12A_Segment | ACACCATCACGACGCGTGGG | 99 | TGG |
| 4 T2 | | | |
| ZC3H12A_Segment | CTACGAGTCTGACGGGATCG | 100 | TGG |
| 4 T3 | | | |
| ZC3H12A_Segment | ACGACGCGTGGGTGGCAAGC | 101 | GGG |
| 4 T7 | | | |

To assess their cutting efficiency in hPSCs, iPS cells were electroporated using the Neon Electroporator (Neon Transfection Kit ThermoFisher Cat #MPK5000) with a ribonucleoprotein (RNP) mixture of Cas9 protein and guide RNA at a molar ratio of 5:1 (gRNA: Cas9) with absolute values of 125 μmol Cas9 and 625 μmol gRNA. To form the RNP complex, gRNA and Cas9 were combined in one vessel with R-buffer (Neon Transfection Kit) to a total volume of 25 μL and incubated for 15 min at RT. Cells were dissociated using ACCUTASER, then resuspended in STEMFLEX™ media (Gibco, cat #11320033), counted using an NC-200 (ChemoMetec) and centrifuged. A total of 1×10⁶ cells were resuspended with the RNP complex and R-buffer was added to a total volume of 125 μL. This mixture was then electroporated with 1 pulse for 20 ms at 1500 V and 1 pulse for 100 ms at 500 V. Following electroporation, the cells were pipetted out into an Eppendorf tube filled with STEMFLEX™ media with REVITACELL™ Supplement (100×). This cell suspension was then plated into tissue culture dishes pre-coated with BIOLAMININ 521 CTG at 1:10 dilution. Cells were cultured in a normoxia incubator (37° C., 8% CO₂) for 48 hours. After 48 hours, genomic DNA was harvested from the cells using QuickExtract (Lucigen, Middleton, WI; Cat #QE09050).

PCR for the target sequences was performed and the resulting amplified DNA was assessed for cutting efficiency by TIDE analysis. PCR for relevant regions was performed using Platinum Taq Supermix (Invitrogen, cat #125320176 and Cat #11495017). The resulting amplicons were submitted for PCR cleanup and Sanger sequencing. Sanger sequencing results were input into Tsunami software along with the guide sequence. Indel percentages and identities were calculated by the software. Particular gRNAs were then selected based on their indel frequency in hPSCs. FAS Ex1 T9 (targeting a sequence comprising SEQ ID NO: 37), CISH Ex1 T18 (targeting a sequence comprising SEQ ID NO: 82), and REGNASE-1 Ex2-T2 (targeting a sequence comprising SEQ ID NO: 94) were chosen for further clone generation due to their high on-target activity.

### Example 12

Generation of IL 15/IL15Rα-P2A-HLA-E Trimer Knock-In, BCMA CAR Knock-In, CIITA Null, B2M Null, ADAM17 Null, FAS Null, CISH Null, and REGNASE-1 Null hPSCs

FAS Ex1 T9 (comprising a spacer sequence of SEQ ID NO: 182), CISH Ex1 T18 (comprising a spacer sequence of SEQ ID NO: 191), and REGNASE-1 Ex2 T2 (comprising a spacer sequence of SEQ ID NO: 203) gRNAs were used to knock-out the FAS, CISH, and REGNASE-1 genes, respectively. IL15/IL15Rα-P2A-HLA-E trimer KI, BCMA CAR KI, CIITA Null, B2M Null, ADAM17 Null cells as described in Examples 9 and 10 were electroporated using the Neon Electroporator with RNP mixtures of Cas9 protein and guide RNA at a molar ratio of 5:1 (gRNA: Cas9) with absolute values of 125 pmol Cas9 and 625 μmol gRNA per 1 million cells. To form the RNP complexes, gRNA and Cas9 were combined in one vessel with R-buffer (Neon Transfection Kit) to a total volume of 25-50 μL and incubated for 15 min at room temperature (RT). This mixture was then combined with the cells to a total volume of ~115 μL using R-buffer. This mixture was then electroporated with 1 pulse for 20 ms at 1500 V followed by 1 pulse for 100 ms at 500 V. Following electroporation, the cells were pipetted out into a 6 well plate filled with STEMFLEX™ media with REVITACELL™ Supplement (100×) and laminin 511. Cells were cultured in a normoxia incubator (37° C., 8% CO₂).

Three to five days post electroporation, the cells were single-cell sorted as described in Example 1. The anti-BCMA CAR antibody (see Table 21) was used for FACS-sorting into 96-well plates. For FACS-sorting, unedited cells served as a negative control. After sorting, the cells were expanded as described in Example 1 and when confluent, samples were split for maintenance and genomic DNA extraction.

For determining indels in the target FAS, CISH, and REGNASE-1 sequences, PCR for relevant regions was performed using Platinum Taq Supermix (Invitrogen, Cat #125320176 and Cat #11495017). The resulting amplicons were submitted for PCR cleanup and Sanger sequencing. Sanger sequencing results were input into Tsunami software along with the guide sequence. The resulting DNA sequences of the target FAS, CISH, and REGNASE-1 regions were aligned in Snapgene software to determine indel identity and homo- or heterozygosity.

Continued expression of BCMA CAR, HLA-E, and IL15 surface proteins was confirmed using fluorescent antibodies as described above in Example 9. Pluripotency of the edited cells was confirmed by detecting OCT4 and SOX2 expression as described above in Example 9. Clone 1 (020 clone 1), homozygous at FAS, CISH, and REGNASE-1 loci, was chosen for further analysis.

### Example 13

Characterization of NK Cells Differentiated from IL15/IL15Rα-P2A-HLA-E Trimer Knock-In, BCMA CAR Knock-In, CIITA Null, B2M Null, ADAM17 Null, FAS Null, CISH Null, and REGNASE-1 Null hPSCs

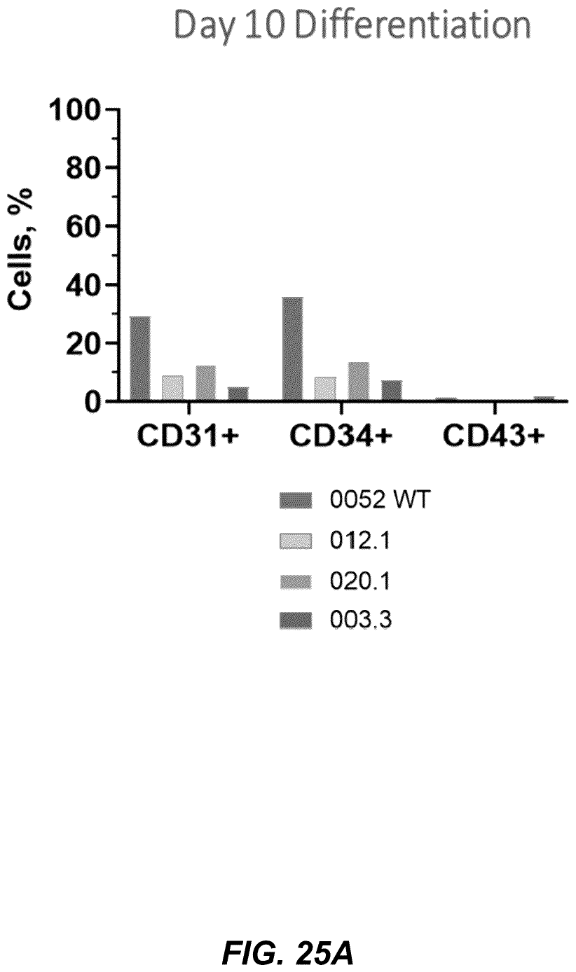

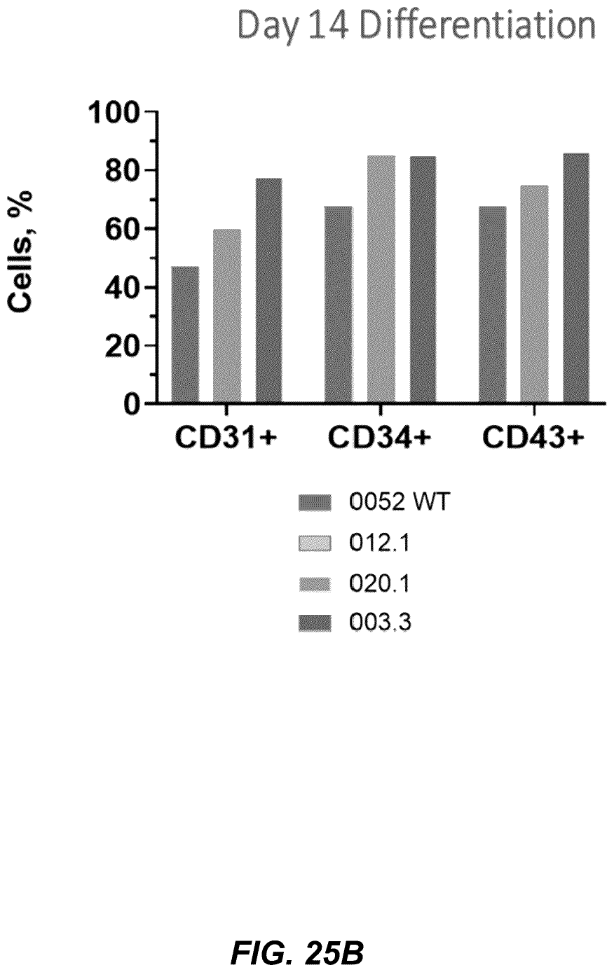

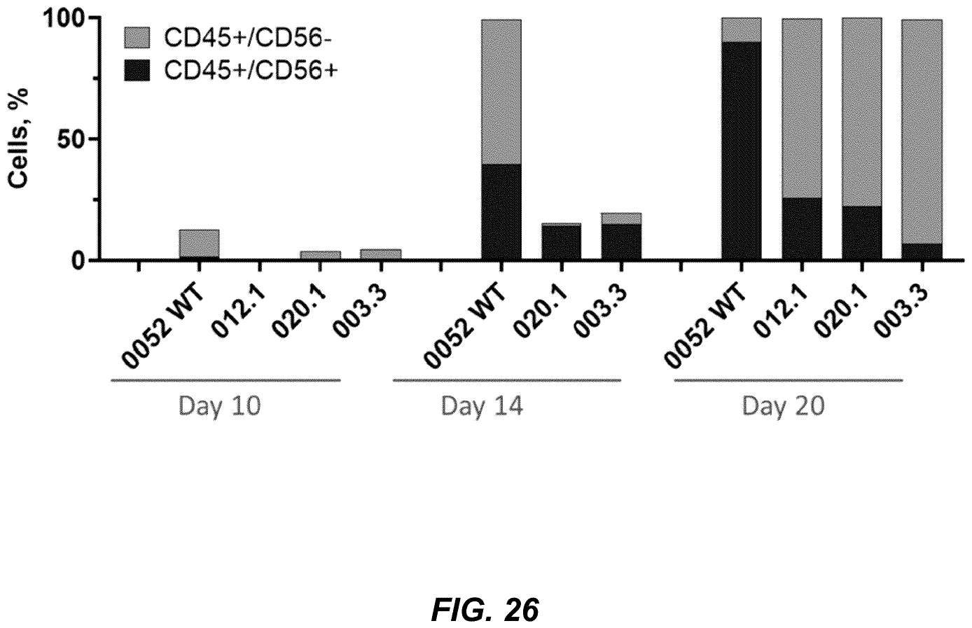

The “020 clone 1” hPSCs (IL15/IL15Rα-P2A-HLA-E trimer knock-in, BCMA CAR knock-in, CIITA null, B2M null, ADAM17 null, FAS null, CISH null, and REGNASE-1 null), as well as “012 clone 1” hPSCs (IL15/IL15Rα-P2A-HLA-E KI, BCMA CAR KI, CIITA null, B2M null, ADAM17 null), “003 clone 3” hPSCS (IL15/IL15Rα-P2A-HLA-E KI, B2M null), and wildtype (WT) were differentiated to iNK cells using Protocol 1, as described in Example 2. Flow cytometry of differentiated cells at Days 10 and 14 showed that the “020 clone 1” differentiated cells had similar patterns of CD31, CD34, and CD43 expression as WT and those differentiated from “012 clone 1” and “003 clone 3” (FIG. 25A-FIG. 25B). Throughout the differentiation process, cells were analyzed for CD45 and CD56 expression by flow cytometry, showing efficient differentiation for all of the edited clones as compared to WT. By day 20, similar levels of the edited clones were CD56+ (FIG. 26). By day 35, more than 99% of the edited clones were CD45+/CD56+.

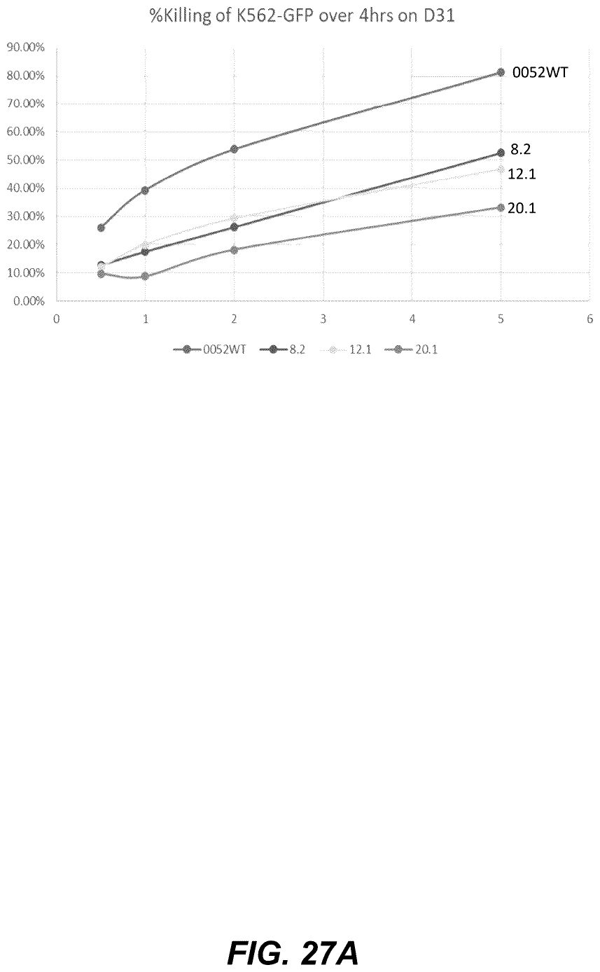

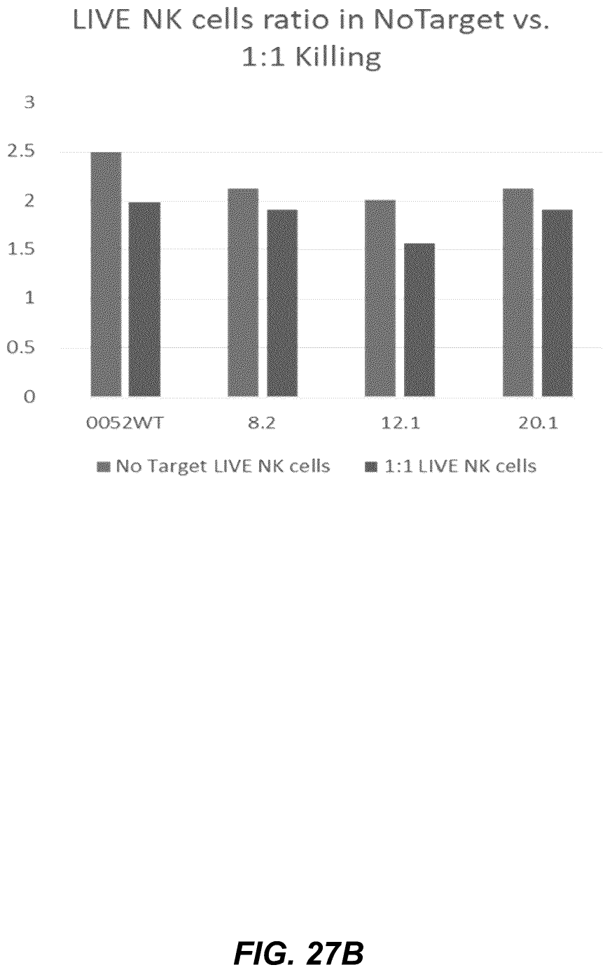

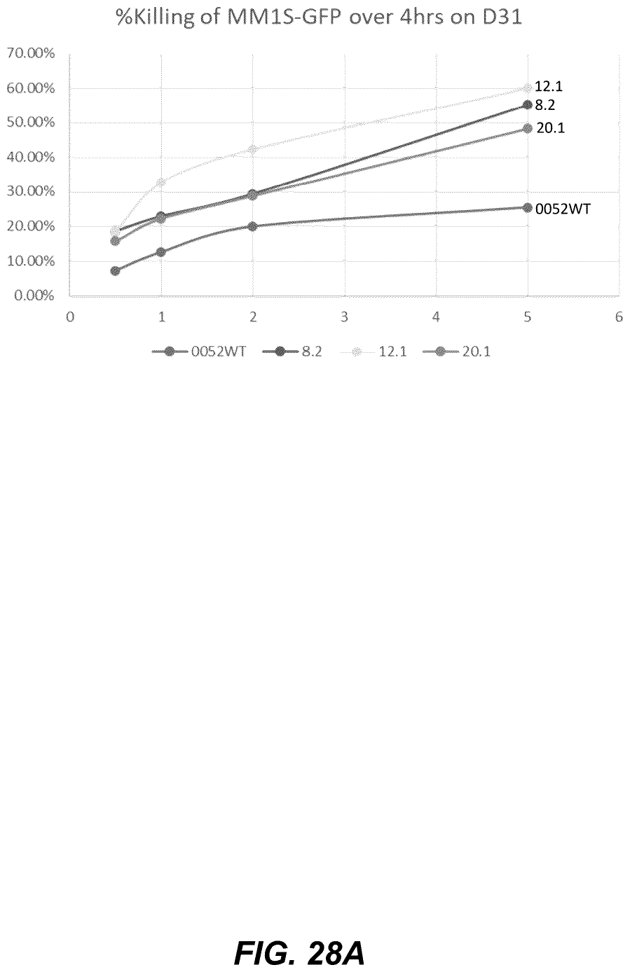

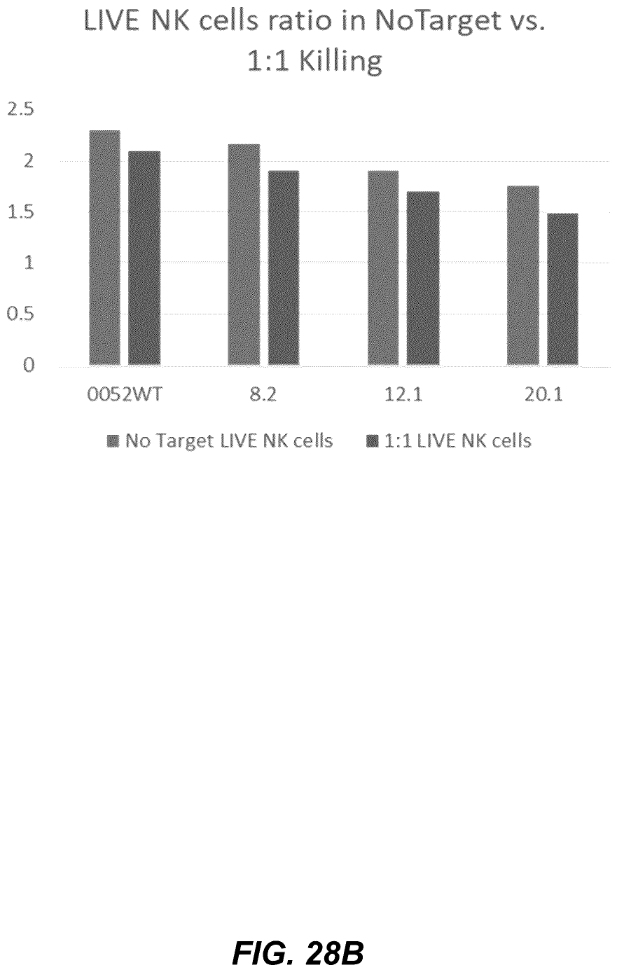

The cytotoxicity of day 31 “020 clone 1” (IL15/IL15Rα-P2A-HLA-E trimer knock-in, BCMA CAR knock-in, CIITA null, B2M null, ADAM17 null, FAS null, CISH null, and REGNASE-1 null), “012 clone 1” (IL15/IL15Rα-P2A-HLA-E KI, BCMA CAR KI, CIITA Null, B2M Null, ADAM17 Null), “008 clone 2” (IL15/IL15Rα-P2A-HLA-E KI, BCMA CAR KI, CIITA null, B2M null), and WT iNK cells towards K562 and MMIS cancer cells was determined using a GFP-based killing assay. The cancer cells were labeled with GFP and killing was monitored over 4 hours. WT cells displayed more effective cytotoxicity against K562 cells than the edited cells (FIG. 27A-FIG. 27B). The “012 clone 1” cells displayed greater cytotoxicity against the BCMA+ expressing MMIS cancer cell line than the WT and other edited cells (FIG. 28A-FIG. 28B).

### Example 14

### Anti-CD30 CAR Development and Selection

Several CD30 CARs were constructed that included variable light and heavy domains from a mouse monoclonal (SEQ ID NOs: 102 and 103, respectively) or a human anti-CD30 antibody (SEQ ID NOs: 104 and 105, respectively), a CD8 transmembrane domain (SEQ ID NO: 122), a CD28 (SEQ ID NO: 123) or 41BB domain (SEQ ID NO: 124), and a CD3Z domain (SEQ ID NO: 125). Table 40 details anti-CD30 CARs.

TABLE 40
ANTI-CD30 CARS

| CAR | Name |
|---|---|
| 1 | Brent_vL_vH_CD28 |
| 2 | 5F11_vH_vL-CD28 |
| 3 | Brent_vL_vH_41BB |
| 4 | Brent_vH-vL_CD28 |
| 5 | 5F11_vH_vL_41BB |
| 6 | 5F11_vL_vH-41BB |
| 7 | Brent_vH_vL_41BB |

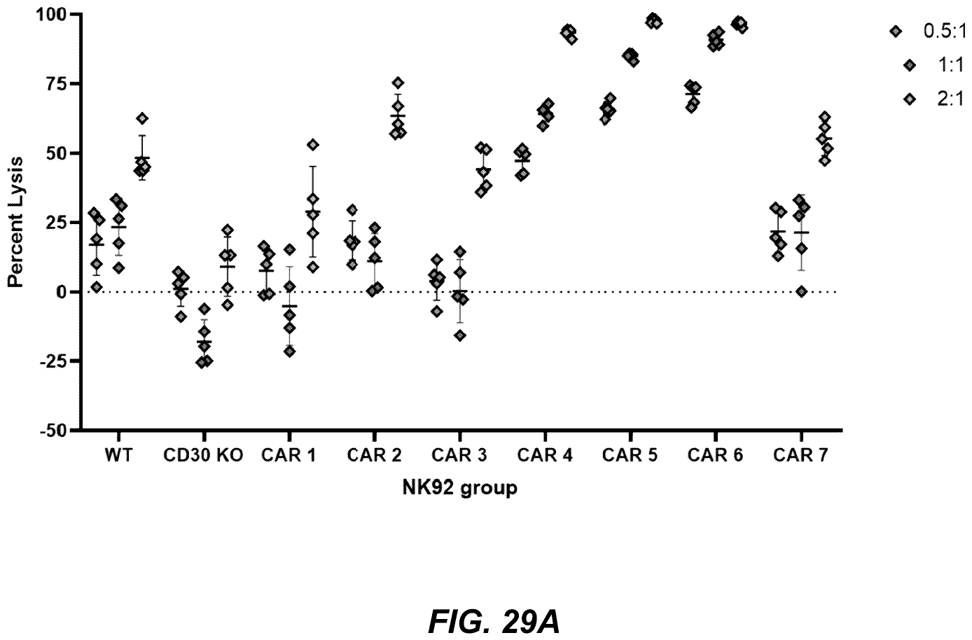

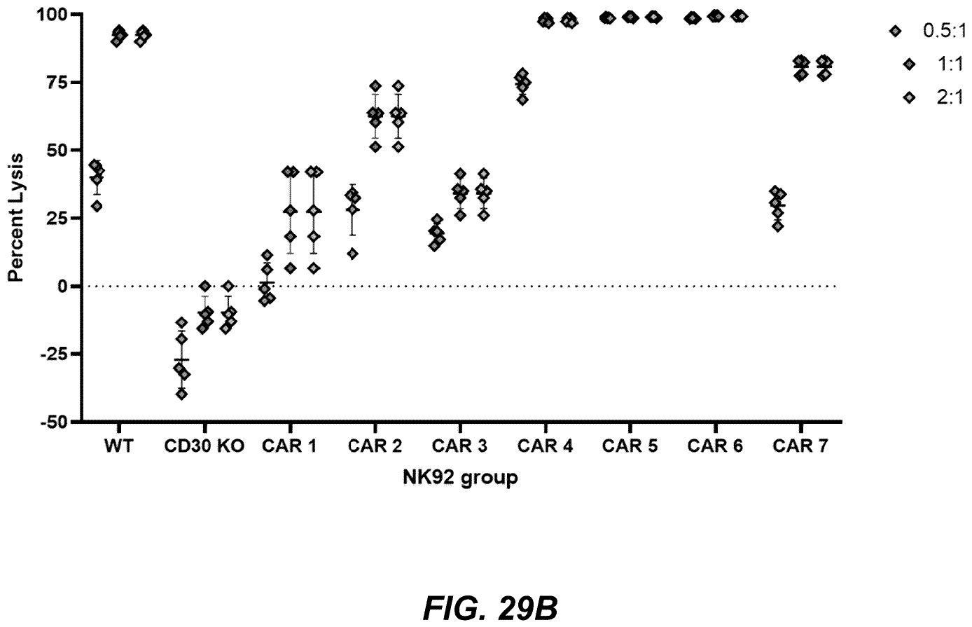

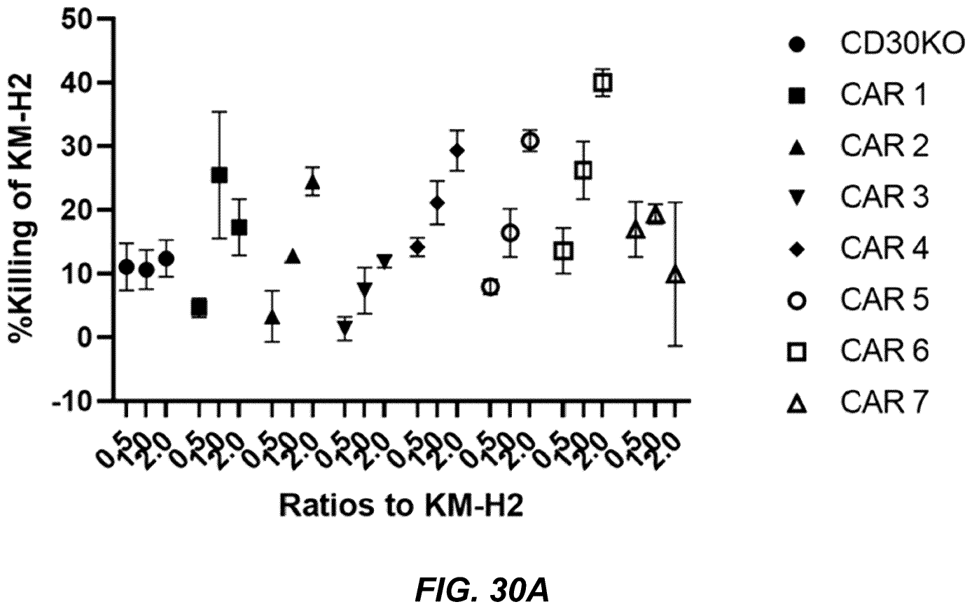

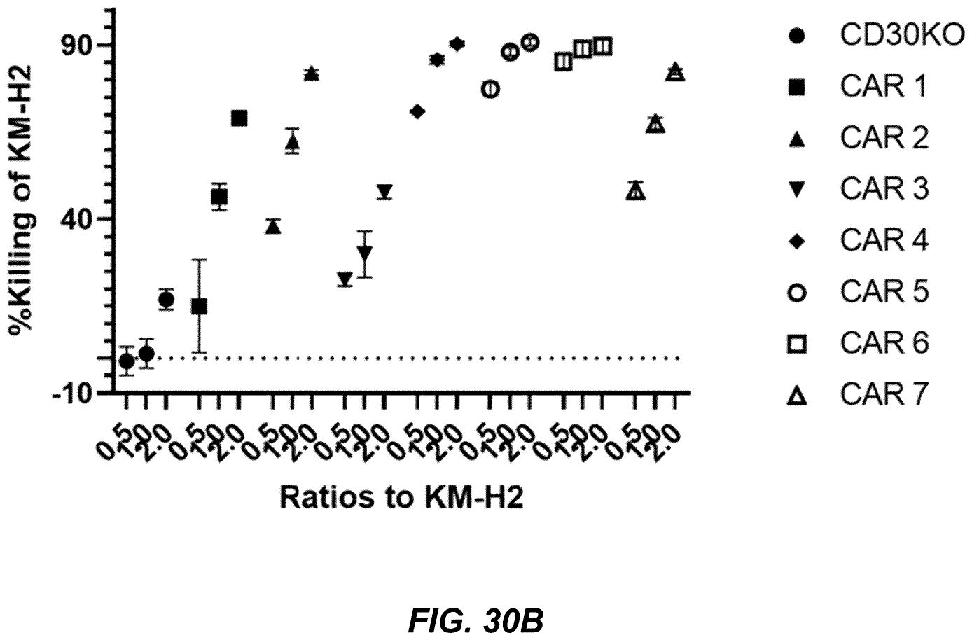

The anti-CD30 CARS were delivered to WT NK92 cells via lentiviral vectors. After selection, cytotoxicity against L428 cancer cell line was determined using a luciferase killing assay. FIG. 29A shows the NK92 anti-CD30 CAR killing results after 4 hours, wherein CARs 4, 5, and 6 outperformed WT at every ratio, with CARs 5 and 6 exhibiting the best killing. CD30 KO strongly reduced NK92 killing ability. FIG. 29B presents the results after 24 hours. CARs 4, 5, and 6 outperformed WT at 0.5:1, with CARs 5 and 6 showing nearly 100% killing for all ratios. Cytotoxicity was also tested against another cancer cell line, KM-H2. FIG. 30A presents results at 4 hours and FIG. 30B shows killing at 24 hours. CARs 4, 5, and 6 showed the best killing. CARs 4, 5, and 6 were chosen for KI into the CIITA gene locus of iPSCs.

### Example 15

Generation of anti-CD30 CAR-P2A-HLA-E Trimer Knock-in, CIITA Null Human Pluripotent Stem Cells

Plasmids were designed to insert anti-CD30 CAR-P2A-HLA-E trimer into the CIITA gene locus essentially as described above in Example 6 (i.e., 86 bp of the CIITA exon 2 would be removed after undergoing HDR). Each donor plasmid contained a CAGGS promoter operably linked to a cDNA of anti-CD30 CAR-P2A-HLA-E trimer flanked by 800 base pair homology arms with identical sequence to the CIITA gene locus around exon 2. The HLA-E trimer cDNA was composed of a B2M signal peptide fused to an HLA-G presentation peptide fused to the B2M membrane protein fused to the HLA-E protein without its signal peptide. The HLA-E trimer coding sequence (including linkers) is SEQ ID NO: 75 (i.e., SEQ ID NOs: 46, 47, 48, 49, 50, and 51). The P2A peptide sequence (SEQ ID NO: 45) connecting the anti-CD30 CAR and the HLA-E trimer allows for the separate expression of both proteins from the single mRNA.

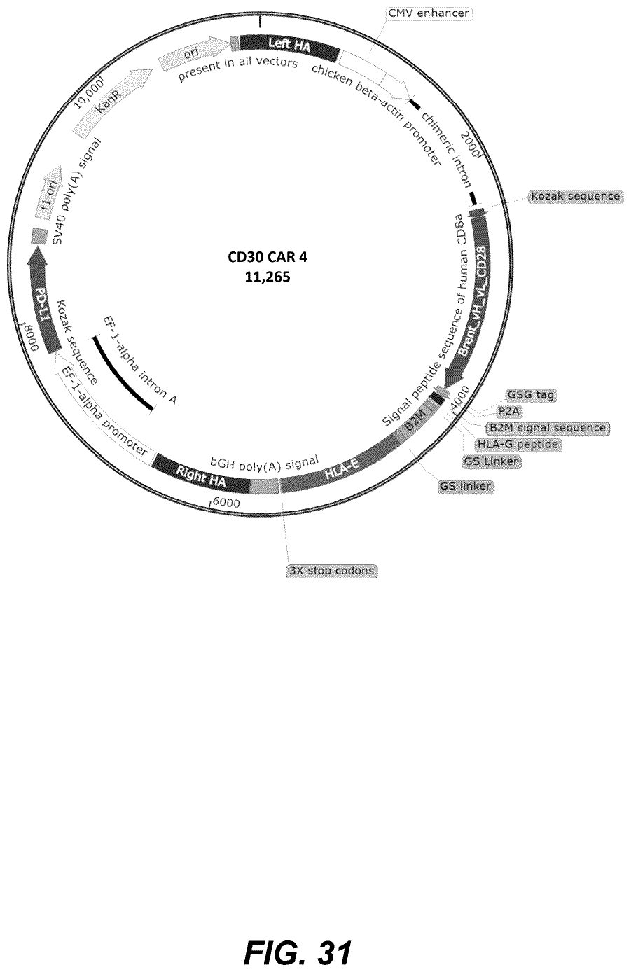

FIG. 31 presents a schematic of an anti-CD30 CAR 4-P2A-HLA-E encoding plasmid (SEQ ID NO: 110) and Table 41 identifies the elements and locations therein. The anti-CD30 CAR 4 coding sequence is SEQ ID NO: 108 (i.e., SEQ ID NOs: 106, 28, 107, and 30) and the anti-CD30 CAR 4 amino acid sequence is SEQ ID NO: 109.

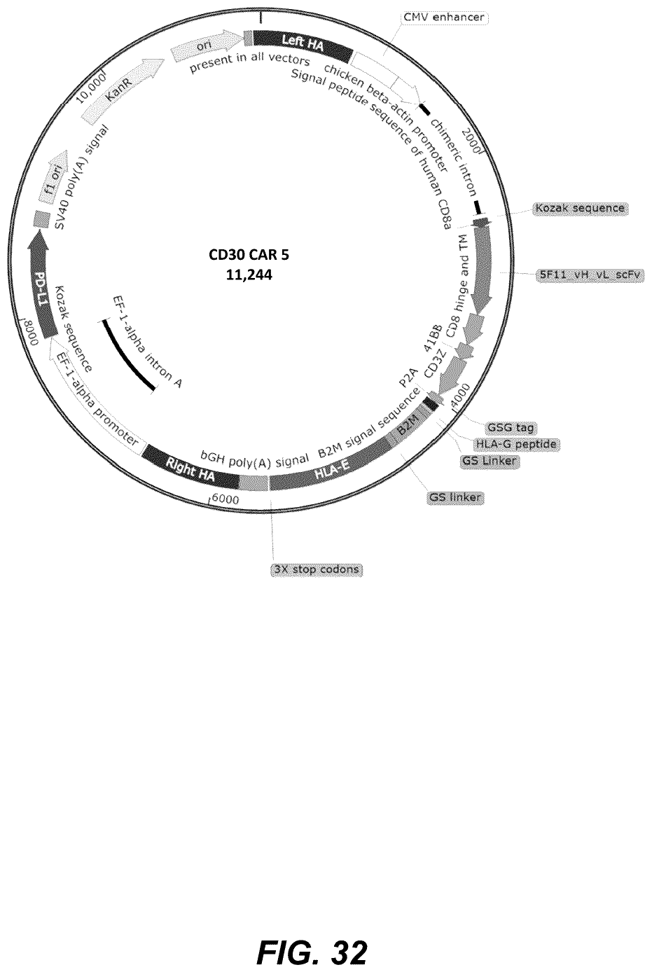

FIG. 32 presents a schematic of an anti-CD30 CAR 5-P2A-HLA-E encoding plasmid (SEQ ID NO: 114) and Table 42 identifies the elements and locations therein. The anti-CD30 CAR 5 coding sequence is SEQ ID NO: 112 (i.e., SEQ ID NOs: 111, 28, 29, and 30) and the anti-CD30 CAR 5 amino acid sequence is SEQ ID NO: 113.

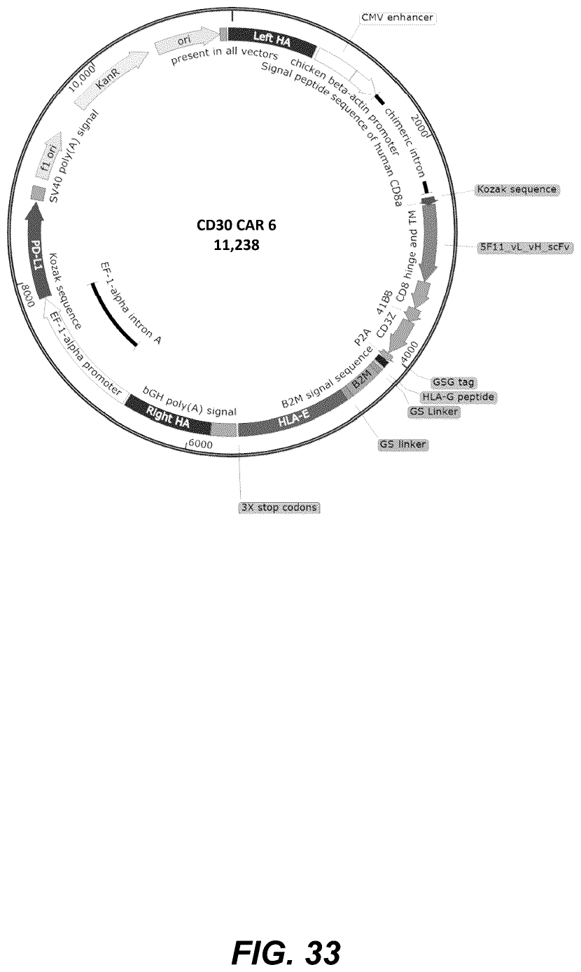

FIG. 33 presents a schematic of an anti-CD30 CAR 6-P2A-HLA-E encoding plasmid (SEQ ID NO: 118) and Table 43 identifies the elements and locations therein. The anti-CD30 CAR 6 coding sequence is SEQ ID NO: 116 (i.e., SEQ ID NOs: 115, 28, 29, and 30) and the anti-CD30 CAR 4 amino acid sequence is SEQ ID NO: 117.

TABLE 41
ELEMENTS OF ANTI-CD30 CAR
4-P2A-HLA-E DONOR PLASMID

| | Location | SEQ |
|---|---|---|
| Element | (size in bp) | ID NO: |
| LHA-CIITA | 11,107-641 (800) | 22 |
| CMV enhancer | 670-1049 (380) | 23 |
| chicken β-actin promoter | 1052-1327 (276) | 24 |
| chimeric intron | 1328-2336 (1009) | 25 |
| CD8a signal peptide | 2381-2443 (63) | 26 |
| Brent_vH_vL | 2444-3172 (729) | 106 |
| CD8TM | 3173-3436 (264) | 126 |
| CD28 domain | 3437-3556 (120) | 107 |
| CD3Z domain | 3557-3892 (336) | 128 |
| GSG tag | 3893-3901 (9) | 44 |
| P2A | 3902-3958 (57) | 45 |
| B2M signal sequence | 3959-4018 (60) | 46 |
| HLA-G peptide | 4019-4045 (27) | 47 |
| GS linker | 4046-4090 (45) | 48 |
| B2M | 4091-4387 (297) | 49 |
| GS linker | 4388-4447 (60) | 50 |
| HLA-E | 4448-5458 (1011) | 51 |
| 3X Stop codons | 5459-5467 (9) | 52 |
| bGH poly(A) signal | 5485-5709 (225) | 31 |
| RHA-CIITA | 5716-6515 (800) | 32 |
| Total plasmid | 11, 265 bp | 110 |

TABLE 42
ELEMENTS OF ANTI-CD30 CAR
5-P2A-HLA-E DONOR PLASMID

| | Location | SEQ |
|---|---|---|
| Element | (size in bp) | ID NO: |
| LHA-CIITA | 11,205-766 (800) | 22 |
| CMV enhancer | 774-1153 (380) | 23 |
| chicken β-actin promoter | 1156-1431 (276) | 24 |
| chimeric intron | 1432-2440 (1009) | 25 |
| CD8a signal peptide | 2485-2547 (63) | 26 |
| 5F11_vH_vL | 2548-3249 (702) | 111 |
| CD8TM | 3250-3513 (264) | 126 |
| 41BB co-stim domain | 3514-3639 (126) | 127 |
| CD3Z domain | 3640-3975 (336) | 128 |
| GSG tag | 3976-3984 (9) | 44 |
| P2A | 3985-4041 (57) | 45 |
| B2M signal sequence | 4042-4101 (60) | 46 |
| HLA-G peptide | 4102-4128 (27) | 47 |
| GS linker | 4129-4173 (45) | 48 |
| B2M | 4174-4470 (297) | 49 |
| GS linker | 4471-4530 (60) | 50 |
| HLA-E | 4531-5541 (1011) | 51 |
| 3X Stop codons | 5542-5550 (9) | 52 |
| bGH poly(A) signal | 5568-5792 (225) | 31 |
| RHA-CIITA | 5799-6598 (800) | 32 |
| Total plasmid | 12,224 | 114 |

TABLE 43
ELEMENTS OF ANTI-CD30 CAR
6-P2A-HLA-E DONOR PLASMID

| | Location | SEQ |
|---|---|---|
| Element | (size in bp) | ID NO: |
| LHA-CIITA | 11,205-766 (800) | 22 |
| CMV enhancer | 795-1174 (380) | 23 |
| chicken β-actin promoter | 1177-1452 (276) | 24 |
| chimeric intron | 1453-2461 (1009) | 25 |
| CD8a signal peptide | 2500-2568 (63) | 26 |
| 5F11_vL_vH | 2569-3270 (700) | 115 |
| CD8TM | 3271-3528 (264) | 126 |
| 41BB co-stim domain | 3529-3654 (126) | 127 |
| CD3Z domain | 3655-3990 (336) | 128 |
| GSG tag | 3991-3999 (9) | 44 |
| P2A | 4000-4056 (57) | 45 |
| B2M signal sequence | 4057-4116 (60) | 46 |
| HLA-G peptide | 4117-4143 (27) | 47 |
| GS linker | 4144-4188 (45) | 48 |
| B2M | 4189-4485 (297) | 49 |
| GS linker | 4486-4545 (60) | 50 |
| HLA-E | 4546-5556 (1011) | 51 |
| 3X Stop codons | 5557-5565 (9) | 52 |
| bGH poly(A) signal | 5583-5807 (225) | 31 |
| RHA-CIITA | 5814-6613 (800) | 32 |
| Total plasmid | 11,238 bp | 118 |

The CIITA-T6 gRNA (target sequence shown in Table 19) was used to facilitate insertion of the anti-CD30 CAR transgenes at the targeted CIITA gene locus. The target sequence of CIITA-T6 is not present in the donor plasmid and thus the donor plasmid itself would not be targeted by this gRNA. CIITA-T6 induced CRISPR cutting in the human genome at exon 2 of CIITA would lead to a frameshift and loss of CIITA protein. Each CD30 CAR donor plasmid was introduced along with a RNP complex made up of the CIITA targeting gRNA and Cas9 protein. Per 1 million of human embryonic stem cells, 2 μg of plasmid DNA was delivered along with the RNP via electroporation. Electroporation was carried out in hiPSC cells using the Neon Electroporator with the RNP mixture of Cas9 protein and guide RNA at a molar ratio of 5:1 (gRNA: Cas9) with absolute values of 125 μmol Cas9 and 625 μmol gRNA per 2 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in STEMFLEX™ media, counted using an NC-200 (ChemoMetec) and centrifuged. A total of 2×10⁶ cells were resuspended with the RNP complex and R-buffer was added to a total volume of 115 μL. This mixture was then electroporated with 3 pulses for 30 ms at 1000 V. Following electroporation, the cells were pipetted out into a well of a 6 well plate filled with STEMFLEX™ media with REVITACELL™ Supplement (100×) and BIOLAMININ 521 CTG at 1:10 dilution. Cells were cultured in a normoxia incubator (37° C., 8% CO₂).

At 2 days post electroporation, the cells were enriched for transfection via fluorescence activated cell sorting (FACS) using an antibody against HLA-E. Then at seven to ten days post electroporation, the cells were enriched for HLA-E trimer knock in cells using an antibody against HLA-E (see Table 21) via fluorescence activated cell sorting (FACS). These enriched cells represent bulk KI population of anti-CD30 CAR-P2A-HLA-E trimer positive cells.

PCR for the genotyping of the edited clones was performed and the resulting amplified DNA was assessed for cutting efficiency by TIDE analysis.

### Example 16

Differentiating Stem Cells into Natural Killer Cells-Protocol 2

It was discovered that some induced pluripotent stem cells did not differentiate efficiently with Protocol 1 described above in Example 1. Thus, Protocol 2 (also called Aligned Process 2.0 or AP2.0) was developed to differentiate these iPSCs into hematopoietic stem and progenitor cells (HSPCs) and then into natural killer (NK) cells. Prior to differentiation, frozen iPSCs were thawed and re-suspended in NK-MED-001a medium (Table 44). Flasks pre-coated with laminin-521 were used for cell culturing. Medium was changed daily using NK-MED-002a (Table 45) medium until cells were used for differentiation.

NK Cell Differentiation. iPS cells were differentiated using the following steps:
1. Day −1 (afternoon), iPSC aggregation: NK-MED-002a medium was aspirated from flasks containing iPSC and the cells were washed with DPBS (no calcium, no magnesium) (Thermo Fisher Scientific, 14190250). DPBS was aspirated and 2 mL ACCUTASE® (Innovative Cell Technologies, AT-104) was added per T25 flask (or 80 μL of ACCUTASER per 1 cm²). Cells were incubated at 37° C. for 3-5 min (not more than 7 minutes). Accutase digested cells were diluted with cold NK-MED-002a medium to a ratio of at least 3:1 (NK-MED-002: ACCUTASE®). Cells were gently resuspended and transferred to a conical tube. Optionally, enough NK-MED-002a medium was added to cells to dilute the ACCUTASE® to a ratio of at least 1:1 and up to 4:1 (NK-MED-002a: ACCUTASER). Cells were pelleted by spinning at 20-300 g for 4 to 5 minutes and re-suspended in 10 mL of NK-MED-003a medium (Table 46). Cells were counted and the cell concentration was diluted to 1×10⁶/mL. 6×10⁶ cells were transferred to another tube and resuspended in a total of 6 mL of NK-MED-003a medium. The cells were transferred to 1 well of ultra-low adhesion 6-well plate (Corning, 3471) and the plate was placed on a platform shaker and rotated at 98 RPM for 18+/−2 hours (overnight).
2. At day 0, morning, at 18+/−2 hours after iPSC aggregation: The plate was rotated in a circular motion to move aggregates towards center of the well and aggregates were collected in a conical tube. Alternatively, all the aggregate solution mix was collected. Aggregates were allowed to settle for 15+/−5 minutes. Cells were resuspended in NK-MED-004 medium (Table 47). The cell number in aggregates was counted. The seeding density was adjusted as needed to resuspend 3×10⁵ cells in aggregates in 2 mL NK-MED-004 medium and plated in one well of a 6-well low adhesion plate. Alternatively, for scale up, an appropriate number of cells was resuspended and transferred to a PBS spinner vessel (PBS Biotech). Seeding density tested for PBS seeding vessel was approximately 1×10⁵ cells per mL per final media volume (day 0+8 hrs). The plate was placed on a platform shaker and rotated at 98 RPM for 8 hours or the PBS spinner vessel were placed on a PBS base (PBS-MINI MagDrive Base Unit; PBS Biotech), in CO₂ incubator with a rotation speed of RPM 38 to 39.
3. At day 0, afternoon, at 8 hours after NK-MED-004 medium addition: 50 mL or 250 mL per well or spinner vessel, respectively, of NK-MED-005c medium (Table 48) was added. The plate was returned to platform shaker or PBS spinner vessel to its base in the CO₂ incubator and left undisturbed until day 2. NK-MED-005c medium components were 2× of their final concentration, therefore it was added to cells in NK-MED-004 at a 1:1 volume ratio.
4. At day 2: NK-MED-005c medium was replaced with NK-MED-006b medium (Table 49).
5. At day 4: NK-MED-006b medium was replaced with NK-MED-007 medium (Table 50).
6. At day 6: NK-MED-007 medium was replaced with NK-MED-008b medium (Table 51), or alternatively: starting at day 6, medium with all aggregates and single cells was transferred into an appropriate volume centrifuge conical tube. Cells were centrifuged and resuspended in NK-MED-008b medium and placed back into original wells and onto platform shaker, or into original vessels and onto base, and returned for continued culture.
7. At day 10: Half or full media change was made with NK-MED-008b medium.
8. At day 14: Full media change was made with NK-MED-009b medium (Table 52).
9. At day 17: One-third media change was made NK-MED-009b medium and then a full media change was made with NK-MED-009b medium.

From day 20 onwards: Starting at day 20, single cell density was estimated from cell culture. A full media change was made with NK-MED-010 medium (Table 53) and cell density adjusted to within 0.8 to 1.5×10⁶ cells/mL. A full media change with NK-MED-010 medium and adjustment of cell density to 0.8-1.5×10⁶ cells/mL was performed every 2-3 days from day 20 to 30.

In the tables below, the volumes are approximate to get the desired concentrations.

TABLE 44
MEDIUM COMPOSITION FOR NK-MED-001A

| | Working | | | Stock |
|---|---|---|---|---|
| Component | Conc. | Volume | | Conc. |
| StemBrew Basal Media | 90% | 980 | mL | 100% |
| StemBrew Supplement | 1X | 20 | mL | 50X |
| Thiazovivin (Biological | 2 μM | 200 | μL | 10 mM |

Industry, 1226056-71-8)

TABLE 45
MEDIUM COMPOSITION FOR NK-MED-002A

| | Working | | | Stock |
|---|---|---|---|---|
| Component | Conc. | Volume | | Conc. |
| StemBrew Basal Media | 90% | 980 | mL | 100% |
| StemBrew Supplement | 1X | 20 | mL | 50X |

TABLE 46
MEDIUM COMPOSITION FOR NK-MED-003A

| Component | Working Conc. | Volume | | Stock Conc. |
|---|---|---|---|---|
| StemBrew Basal | 90% | 979 | mL | 100% |
| StemBrew Supplement | 1X | 20 | mL | 50X |
| Thiazovivin (Biological | 10 μM | 1000 | μL | 10 mM |

Industry, 1226056-71-8)

TABLE 47
MEDIUM COMPOSITION FOR NK-MED-004

| | Working | | | | Stock | |
|---|---|---|---|---|---|---|
| Component | Conc. | | Volume | | Conc. | |
| STEMdiff APEL 2 Medium | 100% | | 999 | mL | 100% | |
| (STEMCELL Technologies, 05275) | | | | | | |
| rh BMP-4 (Peprotech, 120-05ET) | 30 | ng/mL | 300 | μL | 100 | μg/mL |
| Thiazovivin (Biological | 10 | μM | 1000 | μL | 10 | mM |

Industry, 1226056-71-8)

TABLE 48
MEDIUM COMPOSITION FOR NK-MED-005C

| | Working | | | | Stock | |
|---|---|---|---|---|---|---|
| Component | Conc. | | Volume | | Conc. | |
| STEMdiff APEL 2 Medium | 100% | | 998 | mL | 100% | |
| (STEMCELL Technologies, 05275) | | | | | | |
| rh BMP-4 (Peprotech, 120-05ET) | 30 | ng/mL | 300 | μL | 100 | μg/mL |
| rh FGF2 (Peprotech, 100-18C-1MG) | 100 | ng/mL | 1000 | μL | 100 | μg/mL |
| CHIR-99021 (Selleckchem, S1263) | 7 | μM | 700 | μL | 10 | mM |
| Activin-A (R&D Systems, | 5 | ng/mL | 100 | μL | 50 | μg/mL |

338-AC-01M/CF

TABLE 49
MEDIUM COMPOSITION FOR NK-MED-006B

| | Working | | | | Stock | |
|---|---|---|---|---|---|---|
| Component | Conc. | | Volume | | Conc. | |
| STEMdiff APEL 2 Medium | 100% | | 997 | mL | 100% | |
| (STEMCELL Technologies, 05275) | | | | | | |
| rh FGF2 (Peprotech, 100-18C-1MG) | 20 | ng/mL | 200 | μL | 100 | μg/mL |
| rh VEGF165 (Peprotech, 100-20-1MG) | 20 | ng/mL | 200 | μL | 100 | μg/mL |
| rh TPO (Peprotech, 300-18) | 20 | ng/mL | 200 | μL | 100 | μg/mL |
| rh SCF (Peprotech, 300-07) | 100 | ng/mL | 1000 | μL | 100 | μg/mL |
| rh IL-3 (Peprotech, 200-03-100UG) | 40 | ng/mL | 400 | μL | 100 | μg/mL |
| rh Flt3L (Peprotech, 300-19) | 20 | ng/mL | 200 | μL | 100 | μg/mL |
| SB431542 (Selleckchem, S1067) | 5 | μM | 500 | μL | 10 | mM |

TABLE 50
MEDIUM COMPOSITION FOR NK-MED-007

| | Working | | | | Stock | |
|---|---|---|---|---|---|---|
| Component | Conc. | | Volume | | Conc. | |
| STEMdiff APEL 2 Medium | 100% | | 998 | mL | 100% | |
| (STEMCELL Technologies, 05275) | | | | | | |
| rh FGF2 (Peprotech, 100-18C-1MG) | 20 | ng/mL | 200 | μL | 100 | μg/mL |
| rh VEGF165 (Peprotech, 100-20-1MG) | 20 | ng/mL | 200 | μL | 100 | μg/mL |
| rh TPO (Peprotech, 300-18) | 20 | ng/mL | 200 | μL | 100 | μg/mL |
| rh SCF (Peprotech, 300-07) | 100 | ng/mL | 1000 | μL | 100 | μg/mL |
| rh IL-3 (Peprotech, 200-03-100UG) | 40 | ng/mL | 400 | μL | 100 | μg/mL |
| rh Flt3L (Peprotech, 300-19) | 20 | ng/mL | 200 | μL | 100 | μg/mL |

TABLE 51
MEDIUM COMPOSITION FOR NK-MED-008B

| | Working | | | | Stock | |
|---|---|---|---|---|---|---|
| Component | Conc. | | Volume | | Conc. | |
| DMEM (high glucose, GlutaMAX) | 50.3% | | 503 | mL | 100% | |
| (Thermo Fisher, 10566016) | | | | | | |
| F-12 with GlutaMAX | 28% | | 280 | mL | 100% | |
| (Thermo Fisher, 31765035) | | | | | | |
| GlutaMAX (Thermo Fisher, 35050079) | 1X | | 10 | mL | 100X | |
| Glucose* | 4.66 | mM | 4.2 | mL | 1110 | mM |
| Human AB serum | 20% | | 20 | mL | 100% | |
| (Valley Biomedical Inc, HP1022) | | | | | | |
| Zinc sulfate (Millipore Sigma, Z0251) | 36.2 | μM | 978 | μL | 37 | mM |
| Ethanolamine (Millipore Sigma, E0135) | 50 | μM | 3 | μL | 16.6M | |
| Ascorbic acid | 15 | μg/mL | 15 | μL | 10 | mg/mL |
| (Fisher Scientific, NC0762606) | | | | | | |
| Sodium selenite | 5 | ng/mL | 50 | μL | 100 | μg/mL |
| (Millipore Sigma, S9133-1MG) | | | | | | |
| rh IL-3 (Peprotech, 200-03-100UG) | 5 | ng/mL | 50 | μL | 100 | μg/mL |
| rh IL-7 (Peprotech, 200-07) | 20 | ng/mL | 200 | μL | 100 | μg/mL |
| rh Flt3L (Peprotech, 300-19) | 15 | ng/mL | 150 | μL | 100 | μg/mL |
| rh IL-15 (Peprotech, 200-15) | 15 | ng/mL | 150 | μL | 100 | μg/mL |
| rh SCF (Peprotech, 300-07) | 20 | ng/mL | 200 | μL | 100 | μg/mL |

*Total glucose concentration in medium is 20 mM (accounting for glucose in DMEM medium, F12 supplement and added glucose provided here).

TABLE 52
MEDIUM COMPOSITION FOR NK-MED-009B

| | Working | | | | Stock | |
|---|---|---|---|---|---|---|
| Component | Conc. | | Volume | | Conc. | |
| DMEM (high glucose, GlutaMAX) | 50.3% | | 503 | mL | 100% | |
| (Thermo Fisher, 10566016) | | | | | | |
| F-12 with GlutaMAX | 28% | | 280 | mL | 100% | |
| (Thermo Fisher, 31765035) | | | | | | |
| GlutaMAX | 1X | | 10 | mL | 100X | |
| (Thermo Fisher, 35050079) | | | | | | |
| Glucose* | 4.66 | mM | 4.2 | mL | 1110 | mM |
| Human AB serum | 20% | | 20 | mL | 100% | |
| (Valley Biomedical Inc, HP1022) | | | | | | |
| Zinc sulfate (Millipore Sigma, Z0251) | 37 | μM | 978 | μL | 37 | mM |
| Ethanolamine (Millipore Sigma, E0135) | 50 | μM | 3 | μL | 16.6M | |
| Ascorbic acid | 15 | μg/mL | 1500 | μL | 10 | mg/mL |
| (Fisher Scientific, NC0762606) | | | | | | |
| Sodium selenite | 5 | ng/mL | 50 | μL | 100 | μg/mL |
| (Millipore Sigma, S9133-1MG) | | | | | | |
| rh IL-7 (Peprotech, 200-07) | 20 | ng/mL | 200 | μL | 100 | μg/mL |
| rh Flt3L (Peprotech, 300-19) | 15 | ng/mL | 150 | μL | 100 | μg/mL |
| rh IL-15 (Peprotech, 200-15) | 15 | ng/mL | 150 | μL | 100 | μg/mL |
| rh SCF (Peprotech, 300-07) | 20 | ng/mL | 200 | μL | 100 | μg/mL |

*Total glucose concentration in medium is 20 mM (accounting for glucose in DMEM medium, F12 supplement and added glucose provided here).

TABLE 53
MEDIUM COMPOSITION FOR NK-MED-010

| | Working | | | | Stock | |
|---|---|---|---|---|---|---|
| Component | Conc. | | Volume | | Conc. | |
| DMEM (high glucose, GlutaMAX) | 60.5% | | 605 | mL | 100% | |
| F-12 with GlutaMAX | 28% | | 280 | mL | 100% | |
| GlutaMAX | 1X | | 10 | mL | 100X | |
| Glucose* | 2.33 | mM | 2.1 | mL | 1110 | mM |
| Human AB serum | 10% | | 100 | mL | 100% | |
| Zinc sulfate | 37 | μM | 978 | μL | 37 | mM |
| Ethanolamine | 50 | μM | 3 | μL | 16.6M | |
| Ascorbic acid | 15 | μg/mL | 1500 | μL | 10 | mg/mL |
| Sodium selenite | 5 | ng/mL | 50 | μL | 100 | μg/mL |
| Nicotinamide | 6.5 | mM | 6.5 | mL | 1000 | mM |
| rh IL-7 | 10 | ng/mL | 100 | μL | 100 | μg/mL |
| rh Flt3L | 7.5 | ng/mL | 75 | μL | 100 | μg/mL |
| rh IL-15 | 15 | ng/mL | 150 | μL | 100 | μg/mL |
| rh SCF | 20 | ng/mL | 200 | μL | 100 | μg/mL |

*Total glucose concentration in medium is 20 mM (accounting for glucose in DMEM medium, F12 supplement and added glucose provided here).

### Example 17

Generation of Human Pluripotent Stem Cells with SERPINB9-P2A-HLA-E Trimer Knock-in and B2M Knock-out

The SERPINB9-P2A-HLA-E trimer sequence was inserted into a human iPSCs cell line. B2M-2 gRNA (comprising a spacer sequence of SEQ ID NO: 180) was used to facilitate the insertion of the SERPINB9-P2A-HLA-E trimer transgene at the targeted B2M gene locus.

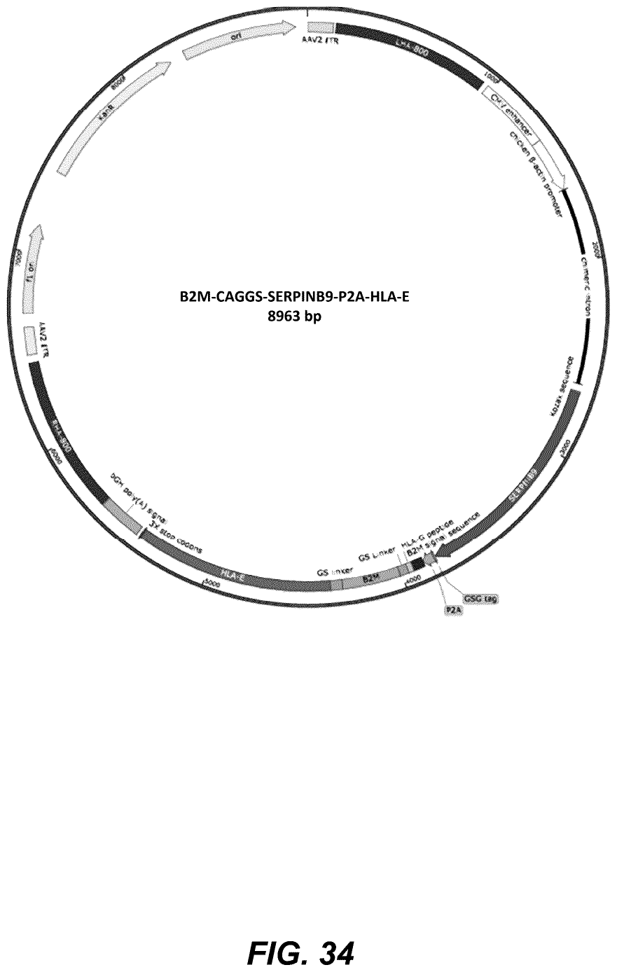

A donor plasmid was designed to insert the SERPINB9-P2A-HLA-E trimer transgene into the B2M gene locus such that the starting codon of B2M was removed after undergoing homology directed repair (HDR) to insert the transgene, nullifying any chance of partial B2M expression. The SERPINB9 and HLA-E trimer sequences were linked by P2A peptide sequences to allow for expression of two separate proteins encoded from a single transcript. FIG. 34 presents a schematic of the donor plasmid (SEQ ID NO: 130) and Table 54 identifies the elements and locations therein. The donor plasmid comprises the SERPINB9-P2A-HLA-E trimer transgene (SEQ ID NO: 131) operably linked to a CAGGS promoter (comprising a CMV enhancer, a chicken β-actin promoter, and a chimeric intron) flanked by 800 base pair homology arms with sequence identity to the B2M gene locus around the target site in exon 1. The HLA-E trimer cDNA was composed of a B2M signal peptide fused to an HLA-G presentation peptide fused to the B2M membrane protein fused to the HLA-E protein without its signal peptide. The HLA-E trimer coding sequence (including linkers) is SEQ ID NO: 75 (i.e., SEQ ID NOs: 46, 4, 48, 49, 50, and 51). This HLA-E trimer design has been previously published (Gornalusse et al. (2017) Nat. Biotechnol. 35 (8): 765-772).

TABLE 54
ELEMENTS OF (B2M) SERPINB9-P2A-
HLA-E TRIMER DONOR PLASMID

| Element | Location (size in bp) | SEQ ID NO: |
|---|---|---|
| Left ITR | 1-130 (130) | 21 |
| LHA-B2M | 145-944 (800) | 36 |
| CMV enhancer | 973-1352 (380) | 23 |
| chicken β-actin promoter | 1355-1630 (276) | 24 |
| chimeric intron | 1631-2639 (1009) | 25 |
| SERPINB9 CDS | 2684-3811 (1128) | 129 |
| GSG tag | 3812-3820 (9) | 44 |
| P2A | 3821-3877 (57) | 45 |
| B2M signal sequence | 3878-3937 (60) | 46 |
| HLA-G peptide | 3938-3964 (27) | 47 |
| GS linker 1 | 3965-4009 (45) | 48 |
| B2M membrane protein | 4010-4306 (297) | 49 |
| GS linker 2 | 4307-4366 (60) | 50 |
| HLA-E CDS | 4367-5377 (1011) | 51 |
| 3X Stop codons | 5378-5386 (9) | 52 |
| bGH poly(A) signal | 5404-5628 (225) | 31 |
| RHA-B2M | 5635-6434 (800) | 54 |
| Right ITR | 6476-6616 (141) | 33 |
| Entire plasmid | (8963) | 130 |

The SERPINB9-P2A-HLA-E trimer donor plasmid was introduced along with a ribonucleoprotein (RNP) complex made up of the B2M targeting gRNA and Cas9 protein. Per 1 million of hiPSC cells, 4 μg of plasmid DNA was delivered along with the RNP via electroporation. Electroporation was carried out in hiPSC cells using the Neon Electroporator with the RNP mixture of Cas9 protein (Biomay) and guide RNA (Biospring) at a molar ratio of 5:1 (gRNA: Cas9) with absolute values of 125 μmol Cas9 and 625 μmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in StemFlex media, counted using an NC-200 (Chemometec) and centrifuged. A total of 2×10⁶ cells were resuspended with the RNP complex and R-buffer was added to a total volume of ~115 μL. This mixture was then electroporated with 3 pulses for 30 ms at 1100 V. Two electroporations was performed. Following electroporation, the cells were pipetted out into a well of a 6 well plate filled with StemFlex media with RevitaCell and laminin 511. The plates were pre-coated with BIOLAMININ 521 CTG at 1:10 dilution. Cells were cultured in a normoxia incubator (37° C., 8% CO₂).

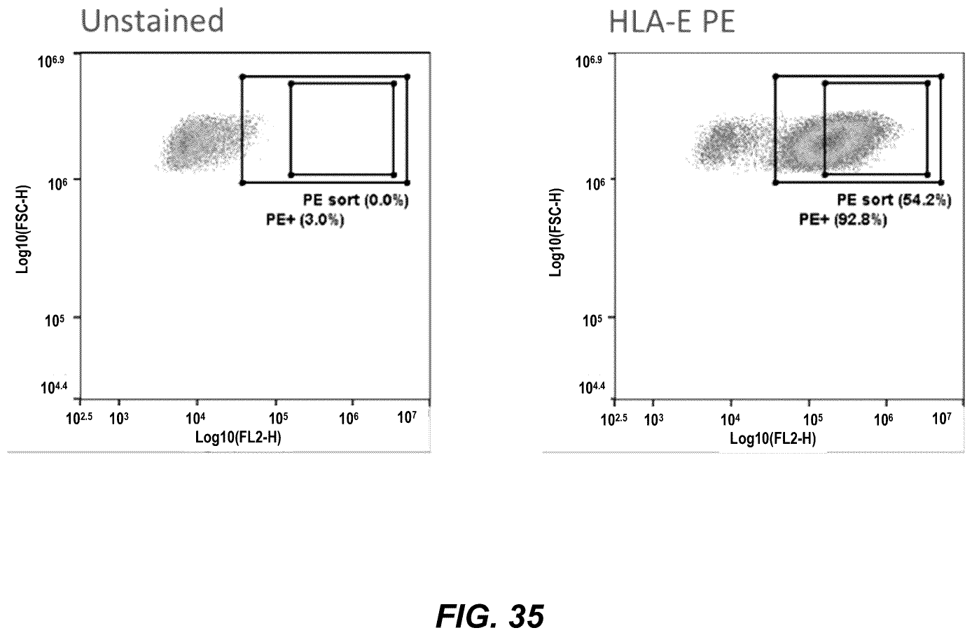

Seven to ten days post electroporation, the cells were enriched for HLA-E trimer expressing cells using an antibody against HLA-E (Table 21) via magnetic assisted cell sorting (MACS) using anti-mouse IgG Dynabeads (ThermoFisher, CELLection™ Pan Mouse IgG Kit, 11531D). These enriched cells represent a bulk KI population of SERPINB9-P2A-HLA-E trimer positive cells. This population was assessed for HLA-E expression by flow cytometry, showing >90% HLA-E expression (FIG. 35).

Following MACS-enrichment, the cells were single-cell sorted as described in Example 1. The anti-HLA-E-PE antibody (Table 21) was used for FACS-sorting into 96-well plates. For FACS-sorting, unedited cells served as a negative control. After sorting, the cells were expanded as described in Example 1 and when confluent, samples were split for maintenance and genomic DNA extraction.

PCR for the genotyping of the edited clones (SERPINB9-P2A-HLA-E trimer knock-in, B2M Null Human Pluripotent Stem Cells (hPSCs)) was performed and the resulting amplified DNA was assessed for cutting efficiency by TIDE analysis.

For determining SERPINB9-P2A-HLA-E trimer knock-in genotyping in the target B2M sequence, PCR for relevant regions was performed using a 2-step protocol with Platinum Taq Supermix (Invitrogen, cat #125320176 and Cat #11495017). The sequences of the PCR primers are presented above in Table 26; and the cycling conditions are provided in Table 27.

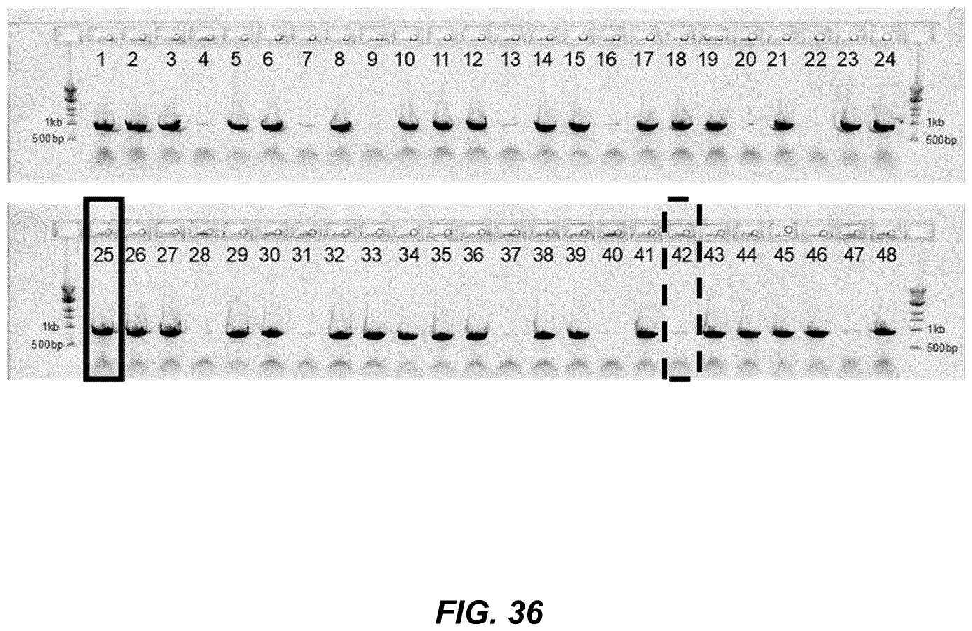

FIG. 36 shows genotyping results of the transgene KI into B2M gene locus for various edited clones. The presence of a 1.1 kb band indicated successful integration of the KI construct into the B2M gene locus, while the absence of a band indicated a WT genotype.

For determining the presence of any unwanted bacterial plasmid elements from the KI plasmid, two PCRs were performed using Platinum Taq Supermix (Invitrogen, cat #125320176 and Cat #11495017). The sequences of the PCR primers are presented in Tables 55 and 57; and the cycling conditions are provided in Tables 56 and 58.

TABLE 55
PLASMID #1 PRIMERS

| | | | SEQ |
|---|---|---|---|
| Name | Type | Sequence (5′-3′) | ID NO: |
| Ori-F2 | forward | CCCTTAACGTGAGTTTTCGTTCCA | 132 |
| | | CTGAGCGTCAGACCCCGTAGAAAA | |
| | | GATCAAAGG | |
| Ori-R | reverse | GTCCAACCCGGTAAGACACGACTT | 133 |
| | | ATCGCCACTGGCAGCAGCCACTGG | |
| | | TAACAG | |

TABLE 56
PLASMID #1 PCR CYCLING PARAMETERS

| | | | |
|---|---|---|---|
| Step | Temperature | Time | Cycles |

| | | | | | |
|---|---|---|---|---|---|
| Denaturation | 98° | C. | 30 | sec | 1 |
| Denaturation | 98° | C. | 10 | sec | 30 |
| Extension | 72° | C. | 10 | sec | |
| Elongation | 72° | C. | 1 | min | 1 |

| | | | |
|---|---|---|---|
| Hold | 4° | C. | hold |

TABLE 57
PLASMID #2 PRIMERS

| | | | SEQ |
|---|---|---|---|
| Name | Type | Sequence (5′-3′) | ID NO: |
| F1-Ori-F | forward | CACTTGCCAGCGCCCTAGCGCC | 134 |
| | | CGCTCCTTTCGCTTTCTTCCCT | |
| | | TCCTTTCTC | |
| F1-Ori-R2 | reverse | GGGCGCGTCAGCGGGTGTTGGC | 135 |
| | | GGGTGTCGGGG | |

TABLE 58
PLASMID #2 PCR CYCLING PARAMETERS

| | | | |
|---|---|---|---|
| Step | Temperature | Time | Cycles |

| | | | | | |
|---|---|---|---|---|---|
| Denaturation | 98° | C. | 30 | sec | 1 |
| Denaturation | 98° | C. | 10 | sec | 30 |
| Extension | 72° | C. | 10 | sec | |
| Elongation | 72° | C. | 1 | min | 1 |

| | | |
|---|---|---|
| Hold | 4 | hold |

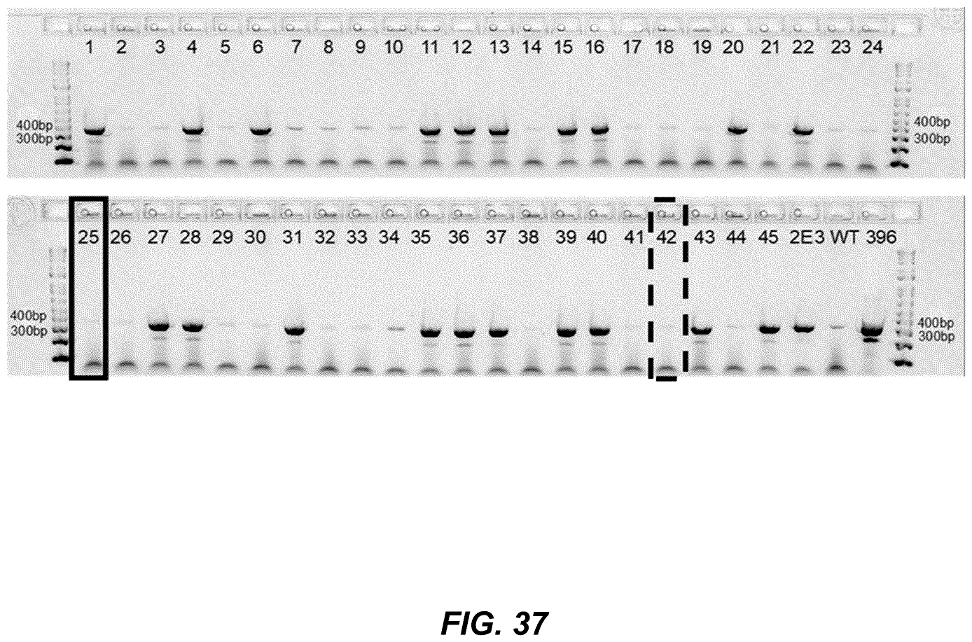

FIG. 37 shows the first PCR amplifying the bacterial plasmid elements that are not supposed to integrate into the genome by HDR because they are outside the homology arms. Both the 5′ and 3′ primers bind outside of the homology arms within the KI plasmid. The presence of a 340 bp band indicates that there is random integration of the plasmid backbone within the genome, clones without bands do not have plasmid insertion.

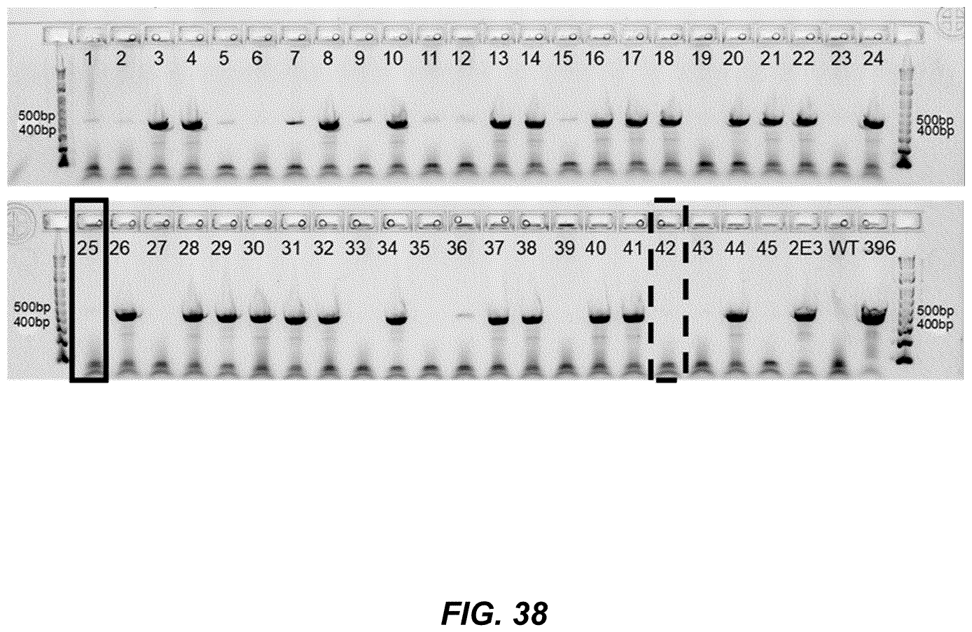

FIG. 38 shows the second PCR amplifying the bacterial plasmid elements outside of the homology arms. The presence of a 476 bp band indicates that there is random integration of the plasmid backbone within the genome, clones without bands do not have plasmid insertion.

For determining indels in the target B2M sequence, PCR for relevant regions was performed using Platinum Taq Supermix (Invitrogen, cat #125320176 and Cat #11495017). The sequences of the PCR primers are presented above in Table 22; and the cycling conditions are provided in Table 23.

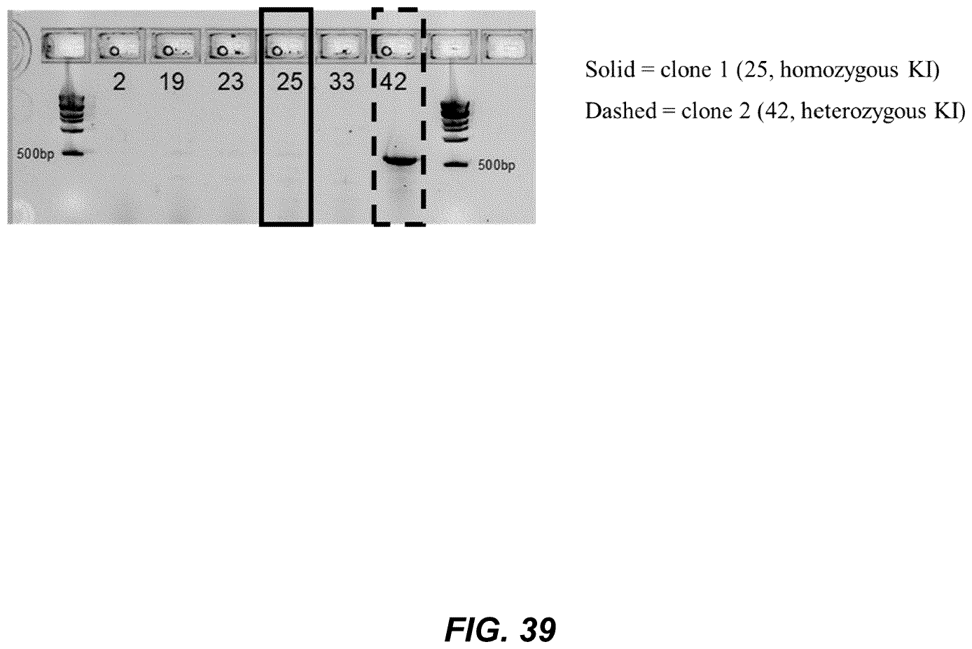

FIG. 39 shows the B2M indel results for various edited clones. The presence of a 573 bp band indicated a WT genotype which would be found in clones that are unedited or are heterozygous for the KI construct, as homozygous clones will not have a band. The B2M KO state of clones was confirmed via PCR and Sanger sequencing. The resulting DNA sequences of the target B2M region were aligned in Snapgene software to determine indel identity and homo- or heterozygosity.

Based on the PCR and Sanger sequencing analysis of the edited clones, the clone shown in lane 25 in FIG. 36-FIG. 39 was chosen as “clone 1” and the clone shown in lane 42 was chosen as “clone 2,” which were shown to have the SERPINB9-P2A-HLA-E KI and no bacterial plasmid elements, while the sequencing data confirmed that B2M was completely knocked-out. Clone 1 was homozygous for the KI into B2M while clone 2 was heterozygous for the KI and had an indel of +1T in the B2M WT band. Clones in lanes 2, 19, 23, and 33 were also chosen as “clones 3-6,” respectively, and were confirmed homozygous for the SERPINB9-P2A-HLA-E KI into B2M.

### Example 18

Differentiation of Stem Cells into Natural Killer Cells

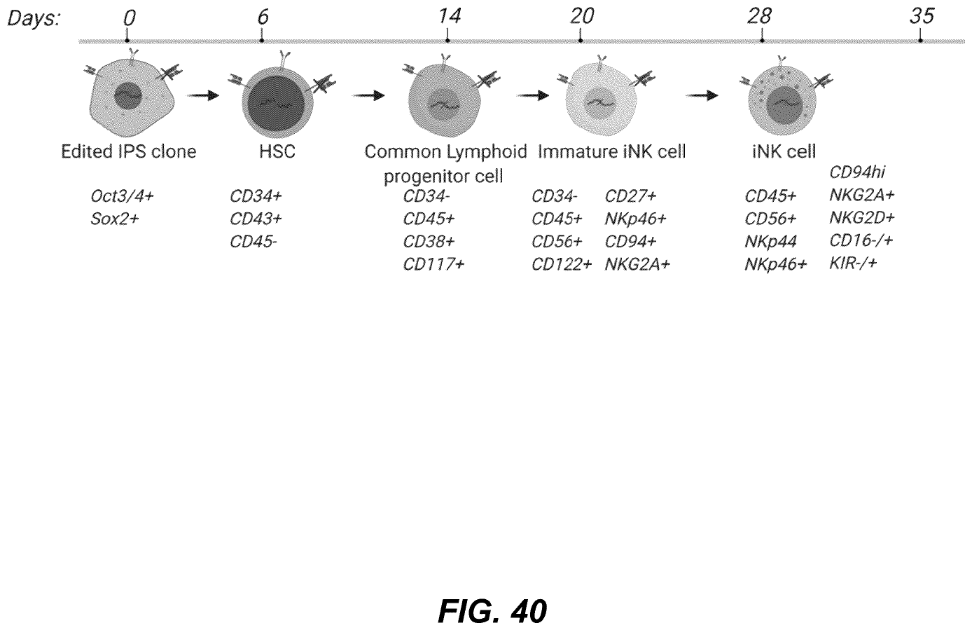

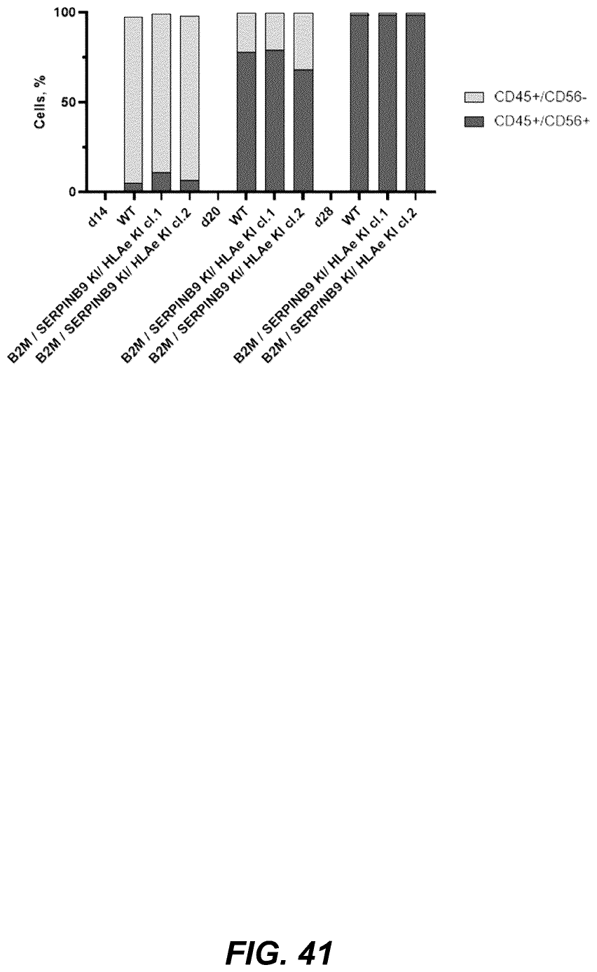

The SERPINB9 KI/HLA-E KI/B2M KO stem cells (clones 1-4) prepared in Example 17, were differentiated into natural killer (NK) cells (iNK cells). FIG. 40 provides a schematic timeline and cell stages of iNK differentiation, as well as the characteristic cell markers at each stage. The iNK differentiation protocol was developed and based on published protocols (see e.g., Ng et al., Nat Protocols 3:768:776 (2008) and U.S. Pat. No. 9,260,696). The INK cells expressed NK cell markers. FIG. 41 presents an example of CD45+/CD56+ iNK cells development during IPSC WT and SERPINB9 KI/HLA-E KI/B2M KO lines differentiation to iNK using the iNK differentiation protocol. Listed edits introduced into IPSC did not affect iNK differentiation.

### Example 19

SERPINB9 Protects Differentiated Cells from NK Cell Killing

The ability of cells differentiated from the SERPINB9 KI stem cells to survive attack from peripheral blood NK (PB-NK) cells was determined using a luminescent cell viability assay (CellTiter-Glo®, Promega). This assay determines the number of viable cells based on quantitation of the ATP present, which signals the presence of metabolically active cells. After incubation with effector cells, the CellTiter-Glo reagent was added to the target cells and luminescence was measured. The light intensity is linearly related to ATP concentration.

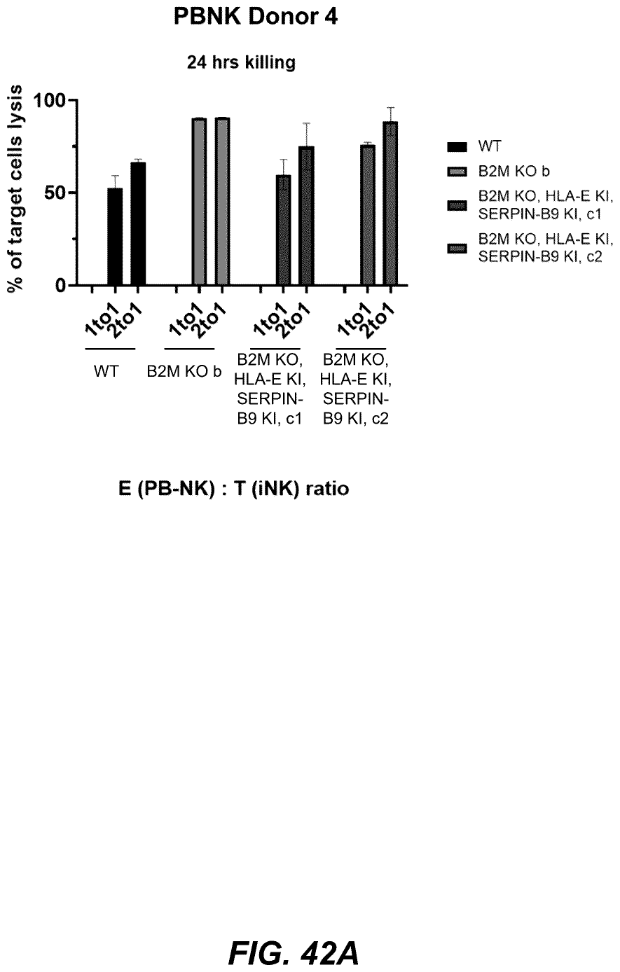

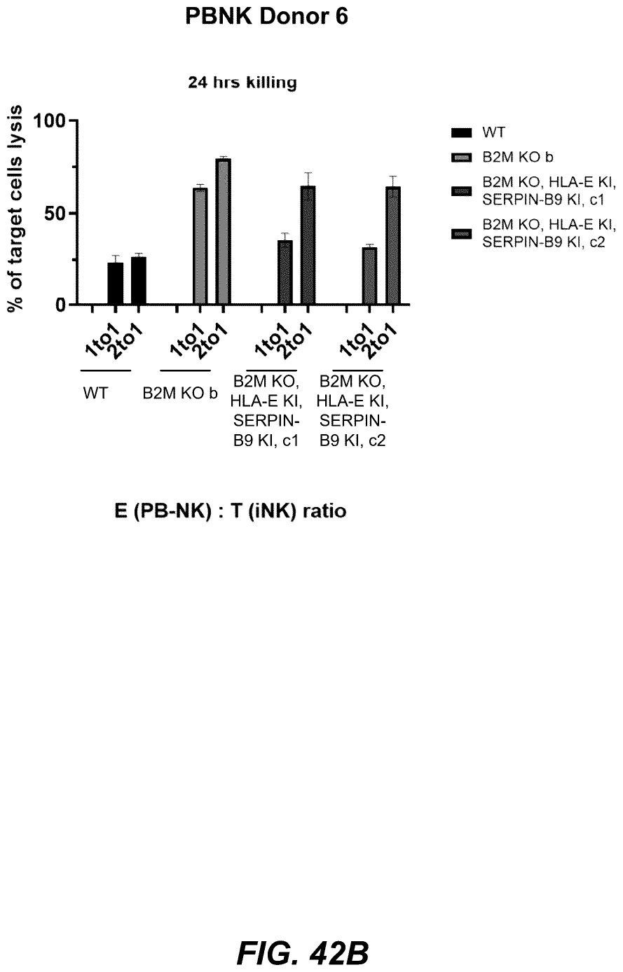

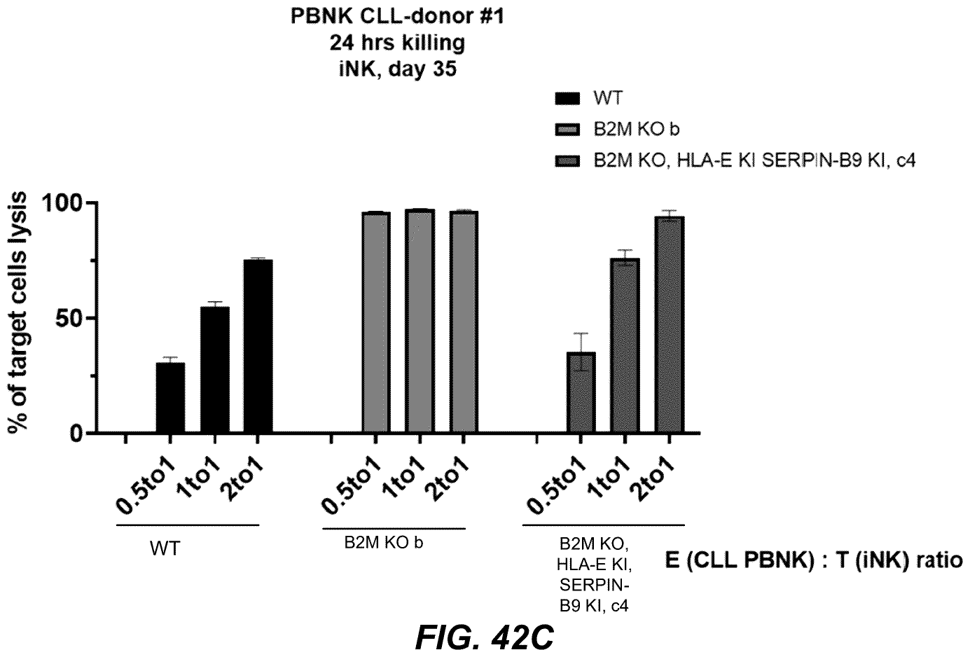

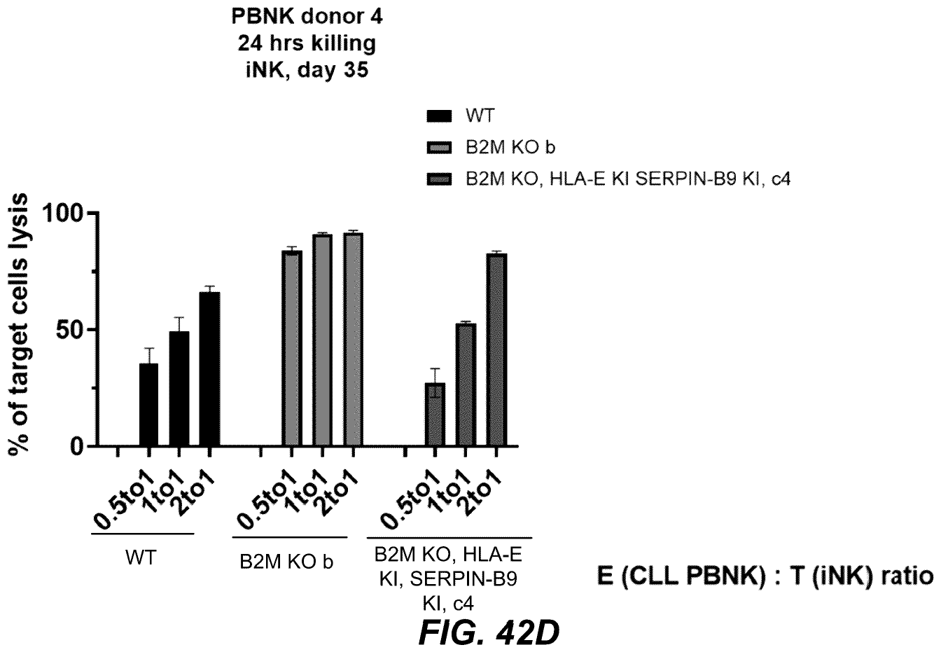

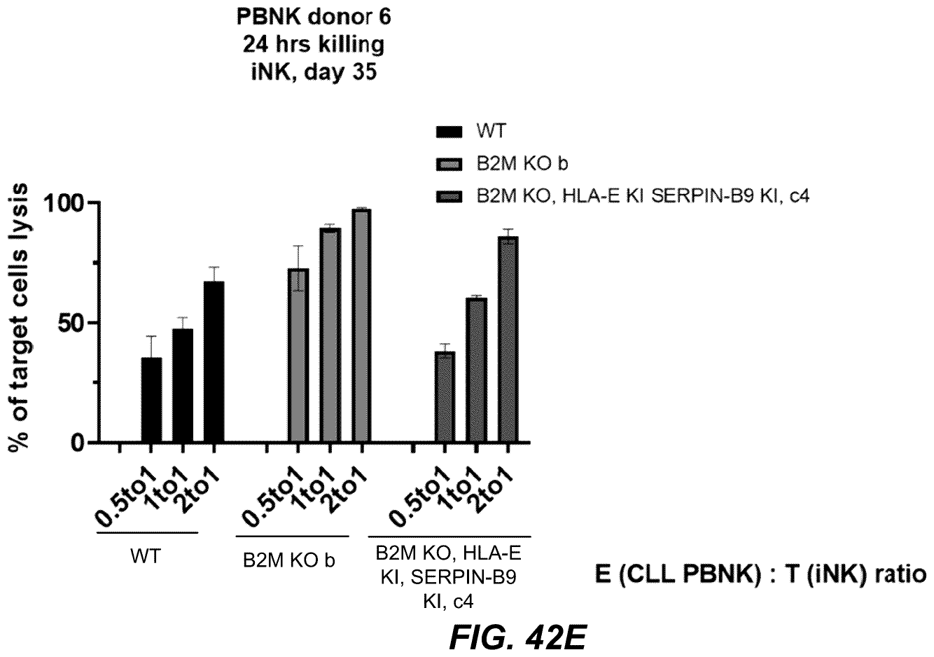

The cytotoxicity of PB-NK cells toward iNK cells differentiated from edited iPSCs was examined. PB-NK effector cells derived from several donors were incubated with day 31 iNK target cells (derived from clones 1 and 2) prepared above in Example 18 at E:T ratios of 1:1 or 2:1 for 18-24 hour. Control target cells included iNK derived from wild-type iPSCs and B2M KO iPSCs. FIG. 42A and FIG. 42B present the percent of target cell lysis in the presence of PB-NK cells from two different donors, PBNK donor 4 (FIG. 42A) and PBNK donor 6 (FIG. 42B), respectively. The B2M KO/SERPINB9 KI/HLA-E KI provided protection from NK killing as compared to B2M KO alone. FIG. 42C-FIG. 42E show the percent of target cell lysis (i.e., day 35 iNK target cells (derived from clone 4) prepared above in Example 3) in the presence of PB-NK cells from 3 different donors, PBNK-CLL-donor #1 (FIG. 42C), PBNK donor 4 (FIG. 42D), and PBNK donor 6 (FIG. 42E), respectively, at E:T ratios of 0.5:1, 1:1 or 2:1 for 24 hours.

### Example 20

Generation of Human Pluripotent Stem Cells with SERPINB9-P2A-IL15/IL15Rα Fusion Knock-In and B2M Knock-Out

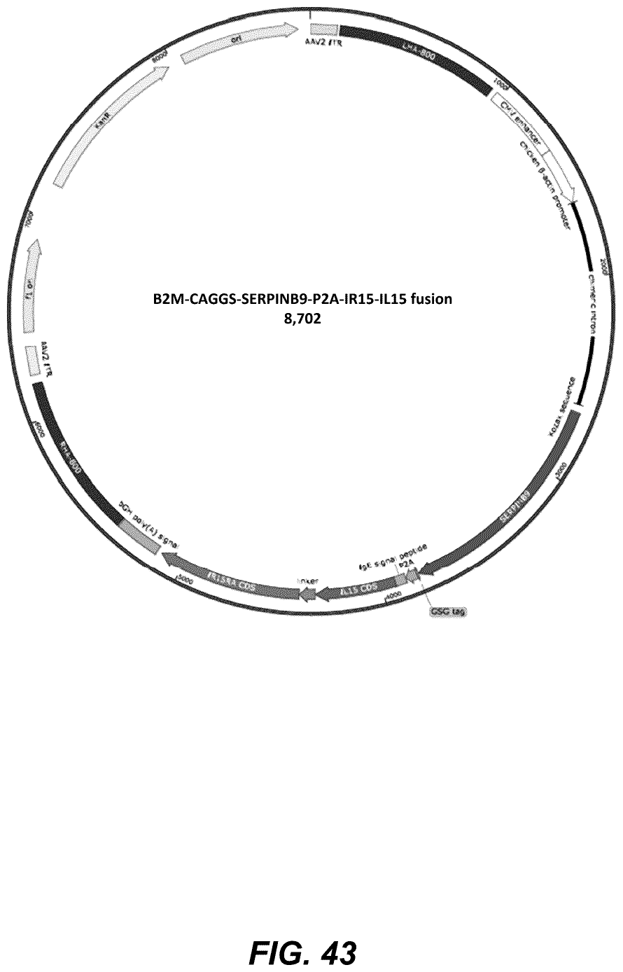

A transgene comprising SERPINB9-P2A-IL15/IL15Rα fusion was inserted in the B2M gene locus of human iPSCs essentially as described above in Example 2. The B2M-2 gRNA (targeting a sequence comprising SEQ ID NO: 34 as shown in Table 19) was used. The donor plasmid was designed such that the starting codon of B2M was removed after undergoing homology directed repair to insert the SERPINB9-P2A-IL15/IL15Rα sequence, nullifying any chance of partial B2M expression. FIG. 43 presents a schematic of the plasmid (SEQ ID NO: 136) and Table 59 identifies the elements and locations therein. The donor plasmid contained a CAGGS promoter driven cDNA of SERPINB9-P2A-IL15/IL15Rα (SEQ ID NO: 137) flanked by 800 base pair homology arms with identical sequence to the B2M gene locus around exon 1. The IL15/IR15α fusion sequence was designed as previously published (Hurton et al. (2016) Proc Natl Acad Sci USA.; 113 (48): E7788-E7797. doi: 10.1073/pnas. 1610544113). The IL15/IR15α fusion coding sequence (including linkers) is SEQ ID NO: 76 (i.e., SEQ ID NOs: 40, 41, 42, and 43).

TABLE 59
ELEMENTS OF (B2M) SERPINB9-P2A-
IL15/IL15RA DONOR PLASMID

| Element | Location (size in bp) | SEQ ID NO: |
|---|---|---|
| Left ITR | 1-130 (130) | 21 |
| LHA-B2M | 145-944 (800) | 36 |
| CMV enhancer | 973-1352 (380) | 23 |
| chicken β-actin promoter | 1355-1630 (276) | 24 |
| chimeric intron | 1631-2639 (1009) | 25 |
| SERPINB9 CDS | 2684-3811 (1128) | 129 |
| GSG tag | 3812-3820 (9) | 44 |
| P2A | 3821-3877 (57) | 45 |
| IgE signal peptide | 3878-3931 (54) | 40 |
| IL15 CDS | 3032-4330 (399) | 41 |
| linker | 4331-4408 (78) | 42 |
| IR15Rα CDS | 4409-5119 (711) | 43 |
| bGH poly(A) signal | 5143-5367 (225) | 31 |
| RHA-B2M | 5379-6173 (800) | 54 |
| Right ITR | 6215-6355 (141) | 33 |
| Entire plasmid | (8702) | 136 |

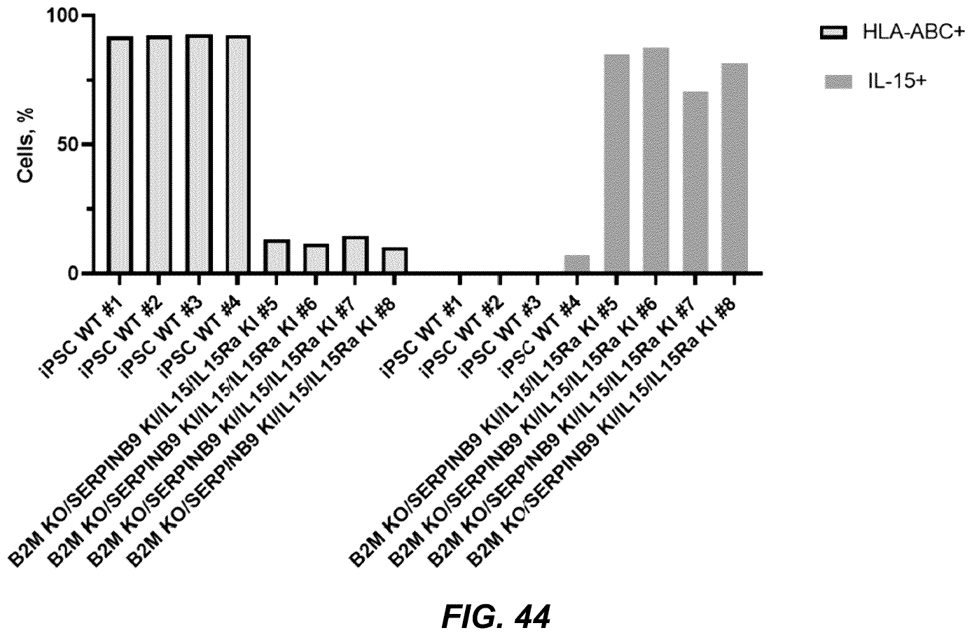

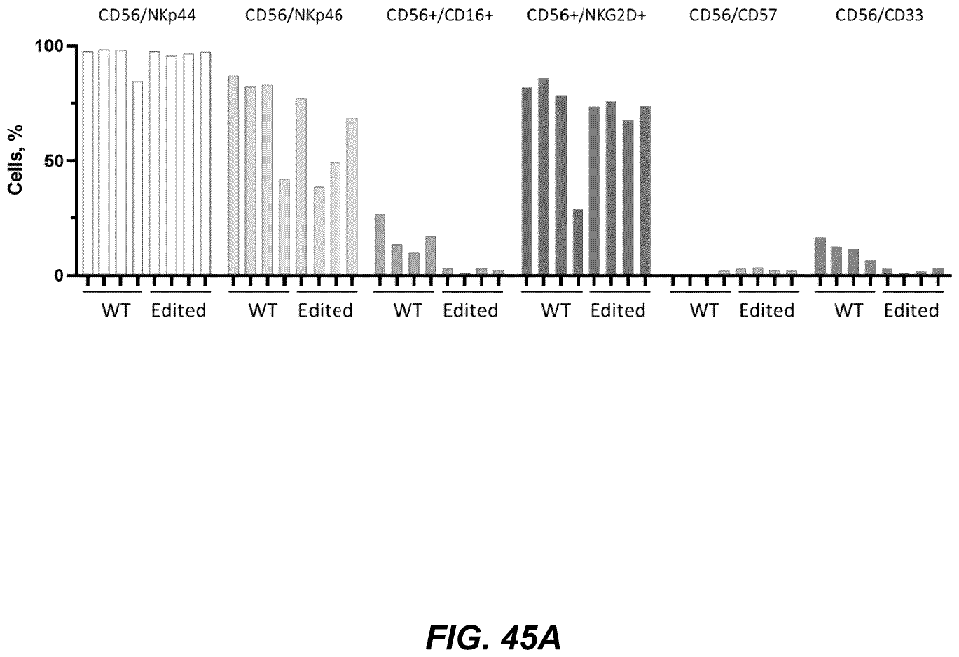

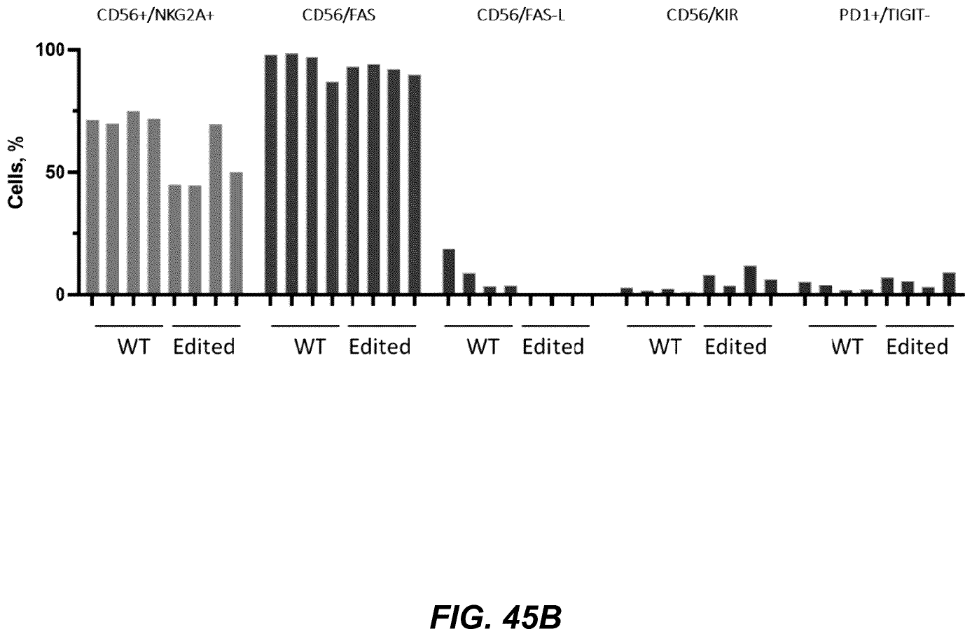

The cells was electroporated with an RNP comprising Cas9 and B2M-2 gRNA and the donor plasmid, cultured, and characterized as described above in Example 17. FIG. 44 shows that the edited cells were effectively edited and maintained in bulk populations. The bulk population of edited cells were differentiated, essentially as described in Example 16. iNK biomarkers were measured on Day 28 (FIG. 45A-FIG. 45B). In a cell killing assay, day 28 and 35 iNK cells had high level of cytotoxicity against K562 cells (4 hr incubation).

After confirmation of the transgene KI and B2M KO, the cells were further edited to have CISH KO (CISH Ex1 T18; targeting a sequence comprising SEQ ID NO: 82) and FAS KO (FAS Ex 1 T9; targeting a sequence comprising SEQ ID NO: 37), and/or differentiated essentially as described above in Example 18.

### Example 21

Generation of f Human Pluripotent Stem Cells with SERPINB9-P2A-IL15/IL15Rα Fusion Knock-In and B2M Knock-Out, Anti-CD30 CAR-P2A-HLA-E Trimer Knock-In and CIITA Knock-Out, CISH Knock-Out, and Fas Knock-Out

iPSCs were generated to have SERPINB9-P2A-IL15/IR15α KI and B2M Knock-out, anti-CD30 CAR-P2A-HLA-E KI and CIITA Knock-out, as described generally in Examples 20 and 15, respectively, with modifications. First, SERPINB9-P2A-IL15/IR15α was knocked into the cells using the SERPINB9-P2A-IL15/IR15α plasmid (SEQ ID NO: 136) and the B2M-T2 gRNA. The iPSCs were passaged the day before electroporation and seeded as 10 million cells per T75 flask. On day of electroporation, the cells were split again and electroporated using the Neon Electroporator with the RNP mixture of Cas9 protein (Biomay) and guide RNA (IDT) at a molar ratio of 5:1 (gRNA: Cas9) with absolute values of 125 pmol Cas9 and 625 pmol gRNA per 2 million cells. To form the RNP complex, gRNA and Cas9 were combined in one vessel with R-buffer (Neon Transfection Kit) to a total volume of 25-50 μL and incubated for 15 min at room temperature (RT). This mixture was then combined with the cells to a total volume of ~115 μL using R-buffer. This mixture was then electroporated with 3 pulses for 30 ms at 1000 V. Following electroporation, the cells were pipetted out into a 6 well plate filled with STEMFLEX™ media with REVITACELL™ Supplement (100×) and BIOLAMININ 521 CTG at 1:10 dilution. Cells were cultured in a normoxia incubator (37° C., 8% CO₂).

On day 2 post electroporation, the cells were FACS-sorted for IL15 positive cells to enrich for transfected cells. At 7 to 10 days post electroporation, the cells were FACS-sorted again for IL15+ cells to enrich for knock in positive cells. The cells were allowed to expand, and then FAS was knocked out using the FAS Ex1 T9 gRNA (targeting a sequence comprising SEQ ID NO: 37). The knockout edits were performed using an RNP of 5:1 (gRNA: Cas9) with absolute values of 125 μmol Cas9 and 625 μmol gRNA per 1 million cells. This mixture was then electroporated with 1 pulse for 20 ms at 1500 V followed by 1 pulse for 100 ms at 500 V. The cells were electroporated with RNP targeting FAS twice 3 days apart to ensure near 100% knockout. Following knockout of FAS, the cells were treated with RNP targeting CISH (CISH Ex1 T18 gRNA (comprising a spacer sequence of SEQ ID NO: 191)) and were also electroporated twice 3 days apart to ensure near 100% knockout of CISH. After this targeting, the bulk population represents an enriched population of SERPINB9-P2A-IL15/IR15α KI cells with a knockout of B2M, FAS, and CISH. This population was expanded and the cells were electroporated with a plasmid encoding anti-CD30 CAR-P2A-HLA-E KI and RNP targeting CIITA. This electroporation for KI was done the same way as the electroporation for KI of SERPINB9-P2A-IL15/IR15α above. At 2 days post electroporation, the cells were enriched for transfection by performing FACS for HLA-E. At 7 to 10 days post electroporation, the cells were FACS sorted again for HLA-E to enrich for knock in positive cells. After this last targeting, the population represents an enriched bulk of SERPINB9-P2A-IL15/IR15α KI and anti-CD30 CAR-P2A-HLA-E KI double positive cells with a knockout of B2M, FAS, CISH, and CIITA. The cells were differentiated essentially as described in Example 18 and characterized.

### Example 22

Generation of Human Pluripotent Stem Cells with SERPINB9-P2A-IL15/IL15Rα Fusion Knock-In and B2M Knock-Out, Anti-GPC3 CAR-P2A-HLA-E Trimer Knock-In and CIITA Knock-Out

iPSCs will be generated to have SERPINB9-P2A-IL15/IR15α KI and B2M KO, anti-GPC3 CAR-P2A-HLA-E KI and CIITA KO, as described generally in Examples 20 and 15 (anti-GPC3 CAR rather than anti-CD30 CAR), respectively, with modifications. First, SERPINB9-P2A-IL15/IR15α will be knocked into the cells using the SERPINB9-P2A-IL15/IR15α plasmid (SEQ ID NO: 136) and the B2M-T2 gRNA. The iPSCs will be passaged the day before electroporation and seeded as 10 million cells per T75 flask. On day of electroporation, the cells will be split again and electroporated using the Neon Electroporator with the RNP mixture of Cas9 protein (Biomay) and guide RNA (IDT) at a molar ratio of 5:1 (gRNA: Cas9) with absolute values of 125 μmol Cas9 and 625 μmol gRNA per 2 million cells. To form the RNP complex, gRNA and Cas9 will be combined in one vessel with R-buffer (Neon Transfection Kit) to a total volume of 25-50 μL and incubated for 15 min at room temperature (RT). This mixture will then be combined with the cells to a total volume of ~115 μL using R-buffer. This mixture will then be electroporated with 3 pulses for 30 ms at 1000 V. Following electroporation, the cells will be pipetted out into a 6 well plate filled with STEMFLEX™ media with REVITACELL™ Supplement (100×) and BIOLAMININ 521 CTG at 1:10 dilution. Cells will be cultured in a normoxia incubator (37° C., 8% CO₂).

On day 2 post electroporation, the cells will be FACS-sorted for IL15 positive cells to enrich for transfected cells. At 7 to 10 days post electroporation, the cells will be FACS-sorted again for IL15+ cells to enrich for knock in positive cells. This population will be expanded and the cells will be electroporated with a plasmid encoding anti-GPC3 CAR-P2A-HLA-E KI and RNP targeting CIITA. This electroporation for KI will be done the same way as the electroporation for KI of SERPINB9-P2A-IL15/IR15α above. At 2 days post electroporation, the cells will be enriched for transfection by performing FACS for HLA-E. At 7 to 10 days post electroporation, the cells will be FACS-sorted again for HLA-E to enrich for knock in positive cells. After this last targeting, the population represents an enriched bulk of SERPINB9-P2A-IL15/IR15α KI and anti-GPC3 CAR-P2A-HLA-E KI double positive cells with a knockout of CIITA. The cells will be differentiated essentially as described in Example 18 and characterized.

### Example 23

Generation of Human Pluripotent Stem Cells with SERPINB9-P2A-IL15/IL15Rα Fusion Knock-In and B2M Knock-Out, Anti-GPR87 CAR-P2A-HLA-E Trimer Knock-In and CIITA Knock-Out

iPSCs will be generated to have SERPINB9-P2A-IL15/IR15α KI and B2M KO, anti-GPR87 CAR-P2A-HLA-E KI and CIITA KO, as described generally in Examples 20 and 15 (anti-GPR87 CAR rather than anti-CD30 CAR), respectively, with modifications. First, SERPINB9-P2A-IL15/IR15α will be knocked into the cells using the SERPINB9-P2A-IL15/IR15α plasmid (SEQ ID NO: 136) and the B2M-T2 gRNA. The iPSCs will be passaged the day before electroporation and seeded as 10 million cells per T75 flask. On day of electroporation, the cells will be split again and electroporated using the Neon Electroporator with the RNP mixture of Cas9 protein (Biomay) and guide RNA (IDT) at a molar ratio of 5:1 (gRNA: Cas9) with absolute values of 125 μmol Cas9 and 625 μmol gRNA per 2 million cells. To form the RNP complex, gRNA and Cas9 will be combined in one vessel with R-buffer (Neon Transfection Kit) to a total volume of 25-50 μL and incubated for 15 min at room temperature (RT). This mixture will then be combined with the cells to a total volume of ~115 μL using R-buffer. This mixture will then be electroporated with 3 pulses for 30 ms at 1000 V. Following electroporation, the cells will be pipetted out into a 6 well plate filled with STEMFLEX™ media with REVITACELL™ Supplement (100×) and BIOLAMININ 521 CTG at 1:10 dilution. Cells will be cultured in a normoxia incubator (37° C., 8% CO₂).

On day 2 post electroporation, the cells will be FACS-sorted for IL15 positive cells to enrich for transfected cells. At 7 to 10 days post electroporation, the cells will be FACS-sorted again for IL15+ cells to enrich for knock in positive cells. This population will be expanded and the cells will be electroporated with a plasmid encoding anti-GPR87 CAR-P2A-HLA-E KI and RNP targeting CIITA. This electroporation for KI will be done the same way as the electroporation for KI of SERPINB9-P2A-IL15/IR15α above. At 2 days post electroporation, the cells will be enriched for transfection by performing FACS for HLA-E. At 7 to 10 days post electroporation, the cells will be FACS-sorted again for HLA-E to enrich for knock in positive cells. After this last targeting, the population represents an enriched bulk of SERPINB9-P2A-IL15/IR15α KI and anti-GPR87 CAR-P2A-HLA-E KI double positive cells with a KO of CIITA. The cells will be differentiated essentially as described in Example 18 and characterized.

### Example 24

Generation of Human Pluripotent Stem Cells with SERPINB9-P2A-IL15/IL15Rα Fusion Knock-In and B2M Knock-Out, Anti-GPC3 or Anti-GPR87 CAR-P2A-HLA-E Trimer Knock-In to Knock-Out CIITA and Additional FAS Knock-Out and CISH Knock-Out

iPSCs will be generated to have SERPINB9-P2A-IL15/IR15α KI and B2M KO, anti-GPC3 or anti-GPR87 CAR-P2A-HLA-E KI and CIITA KO as described generally in Examples 22 and 23. After confirmation of the transgene KI and B2M KO and CIITA KO, the cells will be further edited to have CISH KO (CISH Ex1 T18; targeting a sequence comprising SEQ ID NO: 82) and/or FAS KO (FAS Ex 1 T9; targeting a sequence comprising SEQ ID NO: 37), and differentiated essentially as described above in Example 18.

### Example 25

Generation of Human Pluripotent Stem Cells with SERPINB9-P2A-IL15/IL15Rα Fusion Knock-In and B2M Knock-Out and NKG2D CAR Knock-In into a Second B2M Locus

iPSCs will be generated to have SERPINB9-P2A-IL15/IR15α KI and B2M KO, as described generally in Example 20. A transgene comprising NKG2D CAR will then be inserted into a second B2M site. The NKG2D CAR KI will be confirmed using flow-cytometry. The cells will be differentiated essentially as described in Example 18 and characterized.

### Example 26

Generation of Human Pluripotent Stem Cells with SERPINB9-P2A-IL15/IL15Rα Fusion Knock-In and B2M Knock-Out, NKG2D CAR Knock-In into a Second B2M Locus and Anti-GPC3 or Anti-GPR87 CAR-P2A-HLA-E Trimer Knock-In to Knock-Out CIITA and Additional FAS Knock-Out and CISH Knock-Out

iPSCs will be generated to have SERPINB9-P2A-IL15/IR15α KI and B2M KO, anti-GPC3 or anti-GPR87 CAR-P2A-HLA-E Trimer KI and CIITA KO as previously described in Examples 22 and 23. A transgene comprising NKG2D CAR will then be inserted into a second B2M site. After confirmation of the transgene KI and B2M KO and CIITA KO, the cells will be further edited to have CISH KO (CISH Ex1 T18; targeting a sequence comprising SEQ ID NO: 82) and/or FAS KO (FAS Ex 1 T9; targeting a sequence comprising SEQ ID NO: 37) and differentiated essentially as described above in Example 18.

### Example 27

Generation of Human Pluripotent Stem Cells with SERPINB9-P2A-IL15/IL15Rα Fusion Knock-In and B2M Knock-Out, NKG2D CAR Knock-In into a Second B2M Locus and Anti-GPC3 or Anti-GPR87 CAR-P2A-HLA-E Trimer Knock-In into CIITA Knock-Out and Additional FAS Knock-Out and CISH Knock-Out

iPSCs will be generated to have SERPINB9-P2A-IL15/IR15α KI and B2M KO, anti-GPR87 CAR-P2A-HLA-E KI or anti-GPC3 CAR-P2A-HLA-E KI, and CIITA KO, as described generally in Examples 22 or 23. A transgene comprising NKG2D CAR will then be inserted into a second B2M site. The NKG2D CAR KI will be confirmed using flow-cytometry.

After confirmation of the transgene KI and B2M KO and CIITA KO, the cells will be further edited to have CISH KO (CISH Ex1 T18; targeting a sequence comprising SEQ ID NO: 82) and/or FAS KO (FAS Ex 1 T9; targeting a sequence comprising SEQ ID NO: 37), and differentiated essentially as described above in Example 18.

### Example 28

Generation of Human Pluripotent Stem Cells with SERPINB9-P2A-IL15/IL15Rα Fusion Knock-In and B2M Knock-Out, NKG2D CAR Knock-In into a Second B2M Locus and Anti-GPC3 or Anti-GPR87 CAR-P2A-HLA-E Trimer Knock-In into CIITA Knock-Out, Additional CD16 Inserted into any One of CISH, FAS, FLI1 or TGFβR2 and the Remaining CISH, FAS, FLI1 or TGFβR2 Knocked Out

iPSCs will be generated to have SERPINB9-P2A-IL15/IR15α KI and B2M KO, as described generally in Example 20. A transgene comprising NKG2D CAR will then be inserted into a second B2M site. The NKG2D CAR KI will be confirmed using flow-cytometry. Further anti-GPC3 or anti-GPR87 CAR-P2A-HLA-E trimer KI into CIITA will be generated, making a CIITA KO as previously described in Examples 22 and 23. After confirmation CD16 will be knocked-in these cells at a locus within any one of the CISH, FAS, FLI1 or TGFβR2 genes. The cells will be further edited to KO the remaining CISH, FAS, FLI1 and TGFβR2 genes.

### Example 29

Generation of Human Pluripotent Stem Cells with SERPINB9-P2A-IL15/IL15Rα Fusion Knock-In and B2M Knock-Out, Anti-NKG2D CAR Knock-In into a Second B2M Locus and Anti-GPC3 or Anti-GPR87 CAR-P2A-HLA-E Trimer Knock-In into CIITA Knock-Out, Additional CD64 Inserted into any One of CISH, FAS, FLI1 or TGFβR2 and the Remaining CISH, FAS, FLI1 or TGFβR2 Knocked Out

iPSCs will be generated to have SERPINB9-P2A-IL15/IR15α KI and B2M KO, as described generally in Example 20. A transgene comprising NKG2D CAR will then be inserted into a second B2M site. The NKG2D CAR KI will be confirmed using flow-cytometry. Further anti-GPC3 or anti-GPR87 CAR-P2A-HLA-E trimer KI into CIITA generating a CIITA KO will be generated as previously described in Examples 22 and 23. After confirmation CD64 will be knocked-in these cells at a locus within any one of the CISH, FAS, FLI1 or TGFβR2 genes. The cells will be further edited to KO the remaining CISH, FAS, FLI1 and TGFβR2 genes.

### Example 30

Generation of Human Pluripotent Stem Cells with SERPINB9-P2A-IL15/IL15Rα Fusion Knock-In and B2M Knock-Out, Anti-GPC3 CAR-P2A-HLA-E Trimer Knock-In and CIITA Knock-Out

iPSCs were generated to have SERPINB9-P2A-IL15/IR15α KI and B2M KO, anti-GPC3 CAR-P2A-HLA-E KI and CIITA KO, as described generally in Example 22. SERPINB9-P2A-IL15/IR 15a was knocked into the cells using the SERPINB9-P2A-IL15/IR15α plasmid (SEQ ID NO: 136) and the B2M-T2 gRNA. The iPSCs were passaged the day before electroporation and seeded as 10 million cells per T75 flask. On day of electroporation, the cells were split again and electroporated using the Neon Electroporator with the RNP mixture of Cas9 protein (Biomay) and guide RNA (IDT or Agilent) at a molar ratio of 5:1 (gRNA: Cas9) with absolute values of 125 μmol Cas9 and 625 μmol gRNA per 2 million cells. To form the RNP complex, gRNA and Cas9 were combined in one vessel with R-buffer (Neon Transfection Kit) to a total volume of 25-50 μL and incubated for 15 min at room temperature (RT). This mixture was combined with the cells to a total volume of ~115 μL using R-buffer. This mixture was electroporated with 3 pulses for 30 ms at 1000 V. Following electroporation, the cells were pipetted out into a 6 well plate filled with STEMFLEX™ media with REVITACELL™ Supplement (100×) and BIOLAMININ 521 CTG at 1:10 dilution. Cells were cultured in a normoxia incubator (37° C., 8% CO₂).

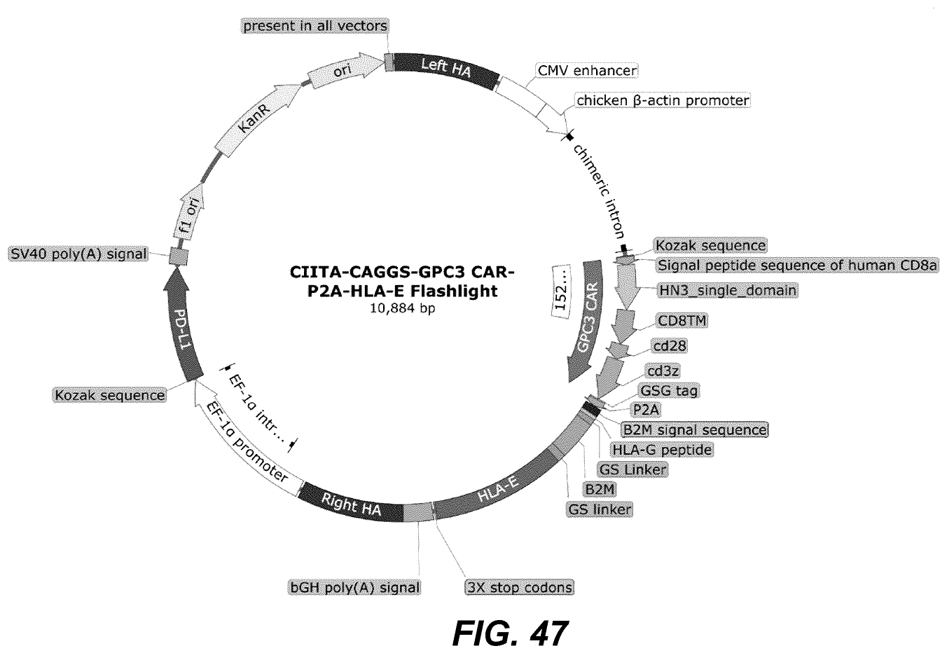

On day 2 post electroporation, the cells were FACS-sorted for IL15 positive cells to enrich for transfected cells. At 7 to 10 days post electroporation, the cells were FACS-sorted again for IL15+ cells to enrich for knock in positive cells. This population was expanded and the cells were electroporated with a plasmid encoding anti-GPC3 CAR-P2A-HLA-E KI and RNP targeting CIITA. FIG. 47 presents a schematic of the plasmid (SEQ ID NO: 156) and Table 60 identifies the elements and locations therein. The donor plasmid contained a CAGGS promoter driven cDNA of anti-GPC3 CAR-P2A-HLA-E flanked by 800 base pair homology arms with identical sequence to the CIITA gene locus around exon 1. The coding sequence of the anti-GPC3 CAR was SEQ ID NO: 152 and the amino acid sequence is SEQ ID NO: 157. The coding sequence of HLA-E is described previously (SEQ ID NO: 75) along with its amino acid sequence (SEQ ID NO: 142).

TABLE 60
ELEMENTS OF (CIITA) ANTI-GPC3
CAR-P2A-HLA-E DONOR PLASMID

| Element | Location (size in bp) | SEQ ID NO: |
|---|---|---|
| Left HA | 10,830-745 (800) | 22 |
| CMV enhancer | 774-1153 (380) | 23 |
| chicken β-actin promoter | 1156-1431 (276) | 24 |
| Chimeric intron | 1432-2440 (1009) | 25 |
| GPC3 CAR | 2485-3615 (1131) | 152 |
| Signal peptide sequence | 2485-2547 (63) | 26 |
| of human CD8a | | |
| HN3_single_domain | 2548-2895 (348) | 153 |
| CD8TM | 2896-3159 (264) | 28 |
| CD28 | 3160-3279 (120) | 154 |
| Cd3z | 3280-3615 (336) | 155 |
| GSG tag | 3616-3624 (9) | 44 |
| P2A | 3625-3681 (57) | 45 |
| B2M signal sequence | 3682-3741 (60) | 46 |
| HLA-G peptide | 3742-3768 (27) | 47 |
| GS Linker | 3769-3813 (45) | 48 |
| B2M | 3814-4110 (297) | 49 |
| GS Linker | 4111-4170 (60) | 50 |
| HLA-E | 4171-5181 (1011) | 51 |
| 3X stop codons | 5182-5190 (9) | 52 |
| bGH poly(A) signal | 5208-5432 (225) | 31 |
| Right HA | 5439-6238 (800) | 32 |
| Entire Plasmid | (10,884) | 156 |

This electroporation for the anti-GPC3 CAR-P2A-HLA-E KI was done the same way as the electroporation for KI of SERPINB9-P2A-IL15/IR15α above. At 2 days post electroporation, the cells were enriched for transfection by performing FACS for HLA-E. At 7 to 10 days post electroporation, the cells were FACS sorted again for HLA-E to enrich for knock in positive cells. After this last targeting, the population represents an enriched bulk of SERPINB9-P2A-IL15/IR15α KI and anti-GPC3 CAR-P2A-HLA-E KI double positive cells with a knockout of CIITA and B2M. The cells will be differentiated essentially as described in Example 18 and characterized.

### Example 31

Generation of Human Pluripotent Stem Cells with SERPINB9-P2A-IL15/IL15Rα Knock-In and B2M Knock-Out, Anti-GPC3 CAR-P2A-HLA-E Knock-In and CIITA Knock-Out and CD64 Knock-In and CISH Knock-Out, and FAS Knock-Out and TGFβR2 Knock-Out

iPSC cells will be generated to have SERPINB9-P2A-IL15/IR15α KI and B2M KO, as described generally in Example 20. Further, anti-GPC3 CAR-P2A-HLA-E trimer KI into CIITA generating a CIITA KO will be generated as previously described in Example 30,

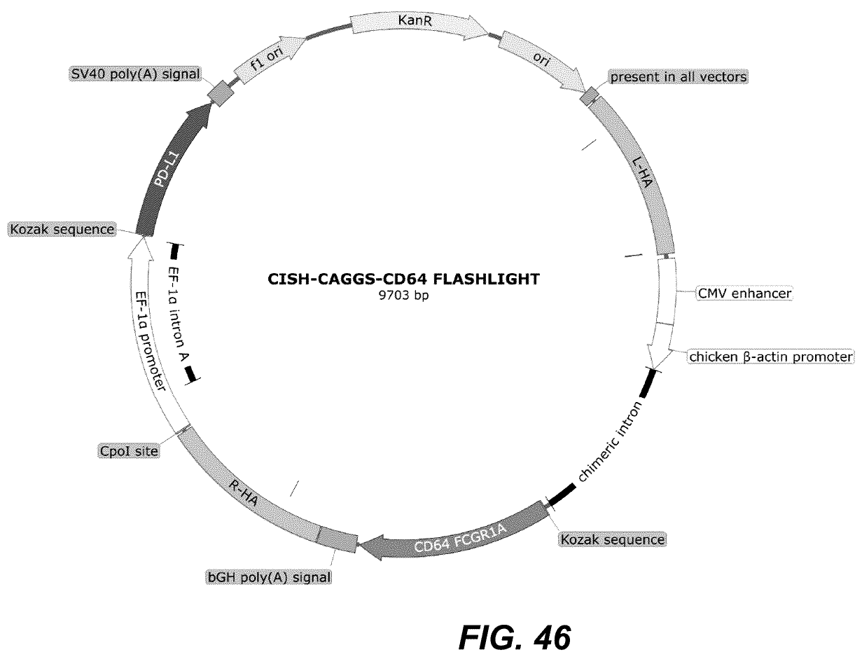

After confirmation, CD64 will be knocked-in these cells at a locus within the CISH gene. FIG. 46 presents a schematic of the plasmid (SEQ ID NO: 149) that will be used and Table 61 identifies the elements and locations therein. The donor plasmid contained a CAGGS promoter driven cDNA of CD64 (SEQ ID NO: 146) flanked by 1000 base pair homology arms with identical sequence to the CISH gene locus around exon 2.

TABLE 61
ELEMENTS OF (CISH) CD64 DONOR PLASMID

| Element | Location (size in bp) | SEQ ID NO: |
|---|---|---|
| Left HA | 1249-2248 (1000) | 145 |
| CMV enhancer | 2277-2656 (380) | 23 |
| chicken β-actin promoter | 2659-2934 (276) | 24 |
| Chimeric intron | 2935-3943 (1009) | 25 |
| CD64 gb | 3884-5373 (1490) | 146 |
| CD64 FCGR1A | 3988-5112 (1125) | 147 |
| bGH poly(A) signal | 5130-5354 (225) | 31 |
| Right HA | 5361-6360 (1000) | 148 |
| Entire Plasmid | (9703) | 149 |

The cells will be further edited to KO the FAS gene (FAS Ex1 T7, targeting a sequence comprising SEQ ID NO: 35; or FAS Ex1 T9, targeting a sequence comprising SEQ ID NO: 37) and TGFβR2 (TGFBRII Ex1-T2, targeting a sequence comprising SEQ ID NO: 151). gRNA target sequences for FAS and TGFβR2 genes are provided in Table 62 below for ease of reference.

TABLE 62
gRNA FOR FAS AND TGFβR2 TARGETING

| | | | |
|---|---|---|---|
| FAS Ex1 T7 | gRNA target | GGATTGCTCA | SEQ ID |
| | sequence in FAS | ACAACCATGC | NO: 35 |
| FAS Ex1 T9 | gRNA target | GATTGCTCAA | SEQ ID |
| | sequence in FAS | CAACCATGCT | NO: 37 |
| TGFBRII | gRNA target in | TGCTGGCGAT | SEQ ID |
| Ex1-T2 | TGFBRII | ACGCGTCCAC | NO: 151 |

### Example 32

Differentiating Stem Cells into Natural Killer Cells—Protocol 3.0

Modified protocol 3.0 (also called Aligned Process 3.0 or AP3.0) was developed as an alternative to the methods described in Examples 3 and 16 to differentiate iPS cells into NK cells. As described below, in some instances NK-MED-001 and NK-MED-002 were prepared using StemFlex medium instead of StemBrew medium.

NK Cell Differentiation. iPS cells were differentiated using the following steps:
1. Day −1 (afternoon), iPSC aggregation: NK-MED-002 medium was aspirated from flasks containing iPSC and the cells were washed with DPBS (no calcium, no magnesium) (Thermo Fisher Scientific, 14190250). DPBS was aspirated and 2 mL ACCUTASE® (Innovative Cell Technologies, AT-104) was added per T25 flask (or 80 μL of ACCUTASE® per 1 cm²). Cells were incubated at 37° C. for 3-5 min (not more than 7 minutes). Accutase digested cells were diluted with cold NK-MED-002 medium to a ratio of at least 3:1 (NK-MED-002: ACCUTASE®). Cells were gently resuspended and transferred to a conical tube. Optionally, enough NK-MED-002 medium was added to cells to dilute the ACCUTASE® to a ratio of at least 1:1 and up to 4:1 (NK-MED-002: ACCUTASER). Cells were pelleted by spinning at 20-300 g for 4 to 5 minutes and re-suspended in 10 mL of NK-MED-003b medium (Table 65). Cells were counted and the cell concentration was diluted to 1×10⁶/mL. 6×10⁶ cells were transferred to another tube and resuspended in a total of 6 mL of NK-MED-003b medium. The cells were transferred to 1 well of ultra-low adhesion 6-well plate (Corning, 3471) and the plate was placed on a platform shaker and rotated at 98 RPM for 18+/−2 hours (overnight).
2. At day 0, morning, at 18+/−2 hours after iPSC aggregation: The plate was rotated in a circular motion to move aggregates towards center of the well and aggregates were collected in a conical tube. Alternatively, all the aggregate solution mix was collected. Aggregates were allowed to settle for 15+/−5 minutes. Cells were resuspended in NK-MED-004b medium (Table 66). The cell number in aggregates was counted. The seeding density was adjusted as needed to resuspend 3×10⁵ cells in aggregates in 2 mL NK-MED-004.1 medium and plated in one well of a 6-well low adhesion plate. Alternatively, for scale up, an appropriate number of cells was resuspended and transferred to a PBS spinner vessel (PBS Biotech). Seeding density tested for PBS seeding vessel was approximately 1×10⁵ cells per mL per final media volume (day 0+8 hrs). The plate was placed on a platform shaker and rotated at 98 RPM for 8 hours or the PBS spinner vessel were placed on a PBS base (PBS-MINI MagDrive Base Unit; PBS Biotech), in CO2 incubator. The rotation speed was adjusted so as to achieve aggregate size of about 80-100 nm.
3. At day 0, afternoon, at 8 hours after NK-MED-004.1 medium addition: 50 mL or 250 mL per well or spinner vessel, respectively, of NK-MED-005c medium (Table 67) was added. The plate was returned to platform shaker or PBS spinner vessel to its base in the CO₂ incubator and left undisturbed until day 2. NK-MED-005c medium components were 2× of their final concentration, therefore it was added to cells in NK-MED-004b at a 1:1 volume ratio.
4. At day 2: NK-MED-005c medium was replaced with NK-MED-006b medium (Table 68).
5. At day 4: NK-MED-006b medium was replaced with NK-MED-007 medium (Table 69).
6. At day 6: NK-MED-007 medium was replaced with NK-MED-008c medium (Table 70), or alternatively: starting at day 6, medium with all aggregates and single cells was transferred into an appropriate volume centrifuge conical tube. Cells were centrifuged and resuspended in NK-MED-008c medium and placed back into original wells and onto platform shaker, or into original vessels and onto base, and returned for continued culture.
7. At day 10: Half or full media change was made with NK-MED-008c medium.
8. At day 14: Full media change was made with NK-MED-009c medium (Table 71).
9. At day 17: One-third media change was made with NK-MED-009c medium and then a full media change was made with NK-MED-009c medium.

From day 20 onwards: Starting at day 20, single cell density was estimated from cell culture. A full media change was made with NK-MED-010b medium (Table 72) and cell density adjusted to within 0.8 to 1.5×10⁶ cells/mL. A full media change with NK-MED-010 medium and adjustment of cell density to 0.8-1.5×10⁶ cells/mL was performed every 2-3 days from day 20 to 30.

TABLE 63
MEDIUM COMPOSITION FOR NK-MED-001

| | Working | | | Stock |
|---|---|---|---|---|
| Component | Conc. | Volume¹ | | Conc. |
| StemBrew Basal Media | 90% | 980 | mL | 100% |
| StemBrew Supplement | 1X | 20 | mL | 50X |
| Thiazovivin (Biological Industry, | 2 μM | 200 | μL | 10 mM |

1226056-71-8)

¹Volumes are approximate to get the desired concentration.

TABLE 64
MEDIUM COMPOSITION FOR NK-MED-002

| Component | Working Conc. | Volume¹ | | Stock Conc. |
|---|---|---|---|---|
| StemBrew Basal Media | 90% | 980 | mL | 100% |
| StemBrew Supplement | 1X | 20 | mL | 50X |

¹Volumes are approximate to get the desired concentration.

TABLE 65
MEDIUM COMPOSITION FOR NK-MED-003B

| Component | Working Conc. | Volume¹ | | Stock Conc. |
|---|---|---|---|---|
| StemBrew Basal | 90% | 979 | mL | 100% |
| StemBrew Supplement | 1X | 20 | mL | 50X |
| Y-27652 | 10 μM | 1000 | μL | 10 mM |

¹Volumes are approximate to get the desired concentration.

TABLE 66
MEDIUM COMPOSITION FOR NK-MED-004B

| | Working | | | Stock |
|---|---|---|---|---|
| Component | Conc. | Volume¹ | | Conc. |
| STEMdiff APEL 2 Medium | 100% | 999 | mL | 100% |
| (STEMCELL Technologies, | | | | |
| 05275) | | | | |
| rh BMP-4 (Peprotech, | 30 ng/mL | 300 | μL | 100 μg/mL |
| 120-05ET) | | | | |

¹Volumes are approximate to get the desired concentration.

TABLE 67
MEDIUM COMPOSITION FOR NK-MED-005C

| | Working | | | | Stock | |
|---|---|---|---|---|---|---|
| Component | Conc. | | Volume¹ | | Conc. | |
| STEMdiff APEL 2 Medium | 100% | | 998 | mL | 100% | |
| (STEMCELL Technologies, 05275) | | | | | | |
| rh BMP-4 (Peprotech, 120-05ET) | 30 | ng/mL | 300 | μL | 100 | μg/mL |
| rh FGF2 (Peprotech, 100-18C-1MG) | 100 | ng/mL | 1000 | μL | 100 | μg/mL |
| CHIR-99021 (Selleckchem, S1263) | 7 | μM | 700 | μL | 10 | mM |
| Activin-A | 5 | ng/mL | 100 | μL | 50 | μg/mL |

(R&D Systems, 338-AC-01M/CF

¹Volumes are approximate to get the desired concentration.

TABLE 68
MEDIUM COMPOSITION FOR NK-MED-006B

| | Working | | | | Stock | |
|---|---|---|---|---|---|---|
| Component | Conc. | | Volume¹ | | Conc. | |
| STEMdiff APEL 2 Medium | 100% | | 997 | mL | 100% | |
| (STEMCELL Technologies, 05275) | | | | | | |
| rh FGF2 (Peprotech, 100-18C-1MG) | 20 | ng/mL | 200 | μL | 100 | μg/mL |
| rh VEGF165 (Peprotech, 100-20-1MG) | 20 | ng/mL | 200 | μL | 100 | μg/mL |
| rh TPO (Peprotech, 300-18) | 20 | ng/mL | 200 | μL | 100 | μg/mL |
| rh SCF (Peprotech, 300-07) | 100 | ng/mL | 1000 | μL | 100 | μg/mL |
| rh IL-3 (Peprotech, 200-03-100UG) | 40 | ng/mL | 400 | μL | 100 | μg/mL |
| rh Flt3L (Peprotech, 300-19) | 20 | ng/mL | 200 | μL | 100 | μg/mL |
| SB431542 (Selleckchem, S1067) | 5 | μM | 500 | μL | 10 | mM |

¹Volumes are approximate to get the desired concentration.

TABLE 69
MEDIUM COMPOSITION FOR NK-MED-007

| | Working | | | | Stock | |
|---|---|---|---|---|---|---|
| Component | Conc. | | Volume¹ | | Conc. | |
| STEMdiff APEL 2 Medium | 100% | | 998 | mL | 100% | |
| (STEMCELL Technologies, 05275) | | | | | | |
| rh FGF2 (Peprotech, 100-18C-1MG | 20 | ng/mL | 200 | μL | 100 | μg/mL |
| rh VEGF165 (Peprotech, 100-20-1MG) | 20 | ng/mL | 200 | μL | 100 | μg/mL |
| rh TPO (Peprotech, 300-18) | 20 | ng/mL | 200 | μL | 100 | μg/mL |
| rh SCF (Peprotech, 300-07) | 100 | ng/mL | 1000 | μL | 100 | μg/mL |
| rh IL-3 (Peprotech, 200-03-100UG) | 40 | ng/mL | 400 | μL | 100 | μg/mL |
| rh Flt3L (Peprotech, 300-19) | 20 | ng/mL | 200 | μL | 100 | μg/mL |

¹Volumes are approximate to get the desired concentration.

TABLE 70
MEDIUM COMPOSITION FOR NK-MED-008C

| | Working | | | | Stock | |
|---|---|---|---|---|---|---|
| Component | Conc. | | Volume¹ | | Conc. | |
| STEMdiff APEL 2 Medium | 100% | | 998 | mL | 100% | |
| (STEMCELL Technologies, 05275) | | | | | | |
| GlutaMAX | 1X | | 10 | mL | 100x | |
| Glucose* | 4.66 | mM | 4.2 | mL | 1110 | mM |
| Human AB serum | 20% | | 200 | mL | 100% | |
| (Valley Biomedical Inc, HP1022) | | | | | | |
| Zinc sulfate | 36.2 | μM | 978 | μL | 37 | mM |
| (Millipore Sigma, Z0251) | | | | | | |
| Ethanolamine | 50 | μM | 3 | μL | 16.6M | |
| (Millipore Sigma, E0135) | | | | | | |
| Ascorbic acid | 15 | μg/mL | 1500 | μL | 10 | mg/mL |
| (Fisher Scientific, NC0762606) | | | | | | |
| Sodium selenite | 5 | ng/mL | 50 | μL | 100 | μg/mL |
| (Millipore Sigma, S9133-1MG) | | | | | | |
| rh IL-3 | 5 | ng/mL | 50 | μL | 100 | μg/mL |
| (Peprotech, 200-03-100UG) | | | | | | |
| rh IL-7 | 20 | ng/mL | 200 | μL | 100 | μg/mL |
| (Peprotech, 200-07) | | | | | | |
| rh Flt3L | 15 | ng/mL | 150 | μL | 100 | μg/mL |
| (Peprotech, 300-19) | | | | | | |
| rh IL-15 | 15 | ng/mL | 150 | μL | 100 | μg/mL |
| (Peprotech, 200-15) | | | | | | |
| rh SCF | 20 | ng/mL | 200 | μL | 100 | μg/mL |

(Peprotech, 300-07)

*Added glucose (e.g., not included in base media).
¹Volumes are approximate to get the desired concentration.

TABLE 71
MEDIUM COMPOSITION FOR NK-MED-009C

| | Working | | | | Stock | |
|---|---|---|---|---|---|---|
| Component | Conc. | | Volume¹ | | Conc. | |
| DMEM (high glucose, GlutaMAX) | 50.3% | | 503 | mL | 100% | |
| (Thermo Fisher, 10566016) | | | | | | |
| F-12 with GlutaMAX | 28% | | 280 | mL | 100% | |
| (Thermo Fisher, 31765035) | | | | | | |
| GlutaMAX | 1X | | 10 | mL | 100X | |
| (Thermo Fisher, 35050079) | | | | | | |
| Glucose* | 4.66 | mM | 4.2 | mL | 1110 | mM |
| Human AB serum | 20% | | 200 | mL | 100% | |
| (Valley Biomedical Inc, HP1022) | | | | | | |
| Zinc sulfate (Millipore Sigma, Z0251) | 37 | μM | 978 | μL | 37 | mM |
| Ethanolamine (Millipore Sigma, E0135) | 50 | μM | 3 | μL | 16.6M | |
| Ascorbic acid (Fisher Scientific, | 15 | μg/mL | 1500 | μL | 10 | mg/mL |
| NC0762606) | | | | | | |
| Sodium selenite (Millipore Sigma, | 5 | ng/mL | 50 | μL | 100 | μg/mL |
| S9133-1MG) | | | | | | |
| rh IL-7 (Peprotech, 200-07) | 20 | ng/mL | 200 | μL | 100 | μg/mL |
| rh Flt3L (Peprotech, 300-19) | 15 | ng/mL | 150 | μL | 100 | μg/mL |
| rh IL-15 (Peprotech, 200-15) | 15 | ng/mL | 150 | μL | 100 | μg/mL |
| rh SCF (Peprotech, 300-07) | 20 | ng/mL | 200 | μL | 100 | μg/mL |

*Total glucose concentration in medium is 20 mM (accounting for glucose in DMEM (high glucose) medium, F12 supplement and added glucose provided here)
¹Volumes are approximate to get the desired concentration.

TABLE 72
MEDIUM COMPOSITION FOR NK-MED-010B

| | Working | | | | Stock | |
|---|---|---|---|---|---|---|
| Component | Conc. | | Volume¹ | | Conc. | |
| DMEM (high glucose, GlutaMAX) | 60.5% | | 605 | mL | 100% | |
| F-12 with GlutaMAX | 28% | | 280 | mL | 100% | |
| GlutaMAX | 1X | | 10 | mL | 100X | |
| Glucose* | 2.33 | mM | 2.1 | mL | 1110 | mM |
| Human AB serum | 10% | | 100 | mL | 100% | |
| Zinc sulfate | 37 | μM | 978 | μL | 37 | mM |
| Ethanolamine | 50 | μM | 3 | μL | 16.6M | |
| Ascorbic acid | 15 | μg/mL | 1500 | μL | 10 | mg/mL |
| Sodium selenite | 5 | ng/mL | 50 | μL | 100 | μg/mL |
| rh IL-7 | 10 | ng/mL | 100 | μL | 100 | μg/mL |
| rh Flt3L | 7.5 | ng/mL | 75 | μL | 100 | μg/mL |
| rh IL-15 | 15 | ng/mL | 150 | μL | 100 | μg/mL |
| rh SCF | 20 | ng/mL | 200 | μL | 100 | μg/mL |

*Total glucose concentration in medium is 20 mM (accounting for glucose in DMEM (high glucose) medium, F12 supplement and added glucose provided here).
¹Volumes are approximate to get the desired concentration.

### Example 33

Differentiating Stem Cells into Natural Killer Cells-Protocol 3.1

Modified protocol 3.0 (also called Aligned Process 3.0 or AP3.0) was performed as described in Example 32 with the following modifications:
1. DMEM/F12 in NK-MED-008c (Table 70) was replaced with APEL medium.
2. Cell conditions in Step 1 were optimized to achieve aggregates that were about 80-100 μm in diameter.

### Example 34

Differentiating Stem Cells into Natural Killer Cells-Protocol 3.2

Modified protocol 3.0 (also called Aligned Process 3.0 or AP3.0) was performed as described in Example 32 with the following modifications:

NK-MED-003, NK-MED-005 and NK-MED-006 media were each modified. The concentration of the ROCK inhibitor in NK-MED-003 was decreased to 5 μM and Y-27632 was used as the ROCK inhibitor. The concentration of the WNT inhibitor (CHIR-99021) in NK-MED-005 was decreased to 3.5 μM. IL-3, FLT3L, and SB431542 were omitted from NK-MED-006b and the concentration of SCF was reduced to 40 ng/ml.

A media switch from NK-MED-007 to NK-MED-008c did not occur at day 6. Instead, cells were cultured in NK-MED-007 for an additional 4 days (6 days total or until day 10, when a media switch to NK-MED-008c occurred).

### Example 35

### Differentiation and Activity of iNK Cells Targeting GPC3

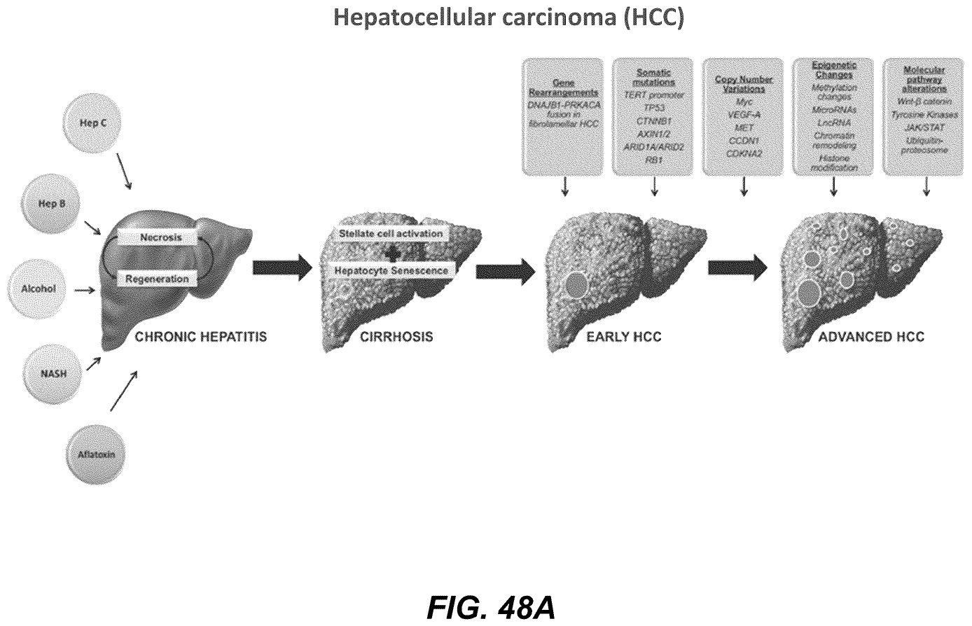

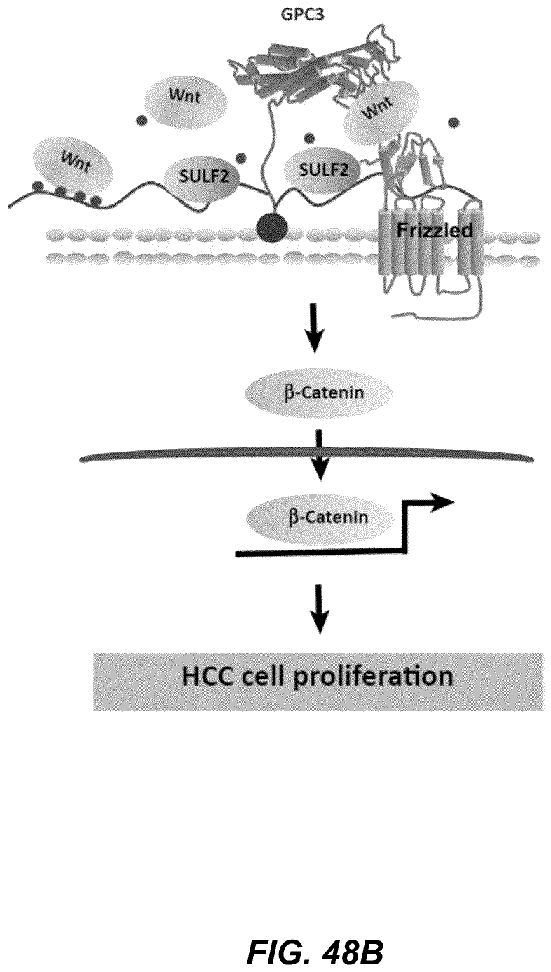

Glypican 3 (GPC3) can be a therapeutic target for cancers such as hepatocellular carcinoma (HCC). Liver cancer is the fourth most common cancer diagnosis and HCC is the most common cause of liver cancer (FIG. 48A). GPC3 is a transmembrane protein that is highly expressed in HCC, and is undetectable in healthy livers or livers with benign disease (FIG. 48B).

iPSC cells were generated to have SERPINB9-P2A-IL15/IR15α KI and B2M KO, as described generally in Example 20. In one embodiment, anti-GPC3 CAR-P2A-HLA-E trimer KI into CIITA generating a CIITA KO was generated, as previously described in Example 30, in the cells that had been modified with a SERPINB9-P2A-IL15/IR15α KI into B2M KO. After confirmation, CD64 was knocked into these cells at a locus within the CISH gene. FIG. 46 presents a schematic of the plasmid (SEQ ID NO: 149) that was used and Table 60 identifies the elements and locations therein. The donor plasmid contained a CAGGS promoter driven cDNA of CD64 (SEQ ID NO: 146) flanked by 1000 base pair homology arms with identical sequence to the CISH gene locus around exon 2. In some embodiments, the cells were further edited to KO the TGFβR2 (TGFβRII Ex1-T2, targeting a sequence comprising SEQ ID NO: 151) and the FAS gene (FAS Ex1 T7, targeting a sequence comprising SEQ ID NO: 35 or FAS Ex1 T9, targeting a sequence comprising SEQ ID NO: 37). In another embodiment, cells comprising the SERPINB9-P2A-IL15/IR15α KI into B2M KO were then edited by knock-in of CD64 into the CISH gene locus. Then, the cells were further edited to KO the FAS gene. These cells were then edited to insert anti-GPC3 CAR-P2A-HLA-E trimer KI into CIITA gene locus.

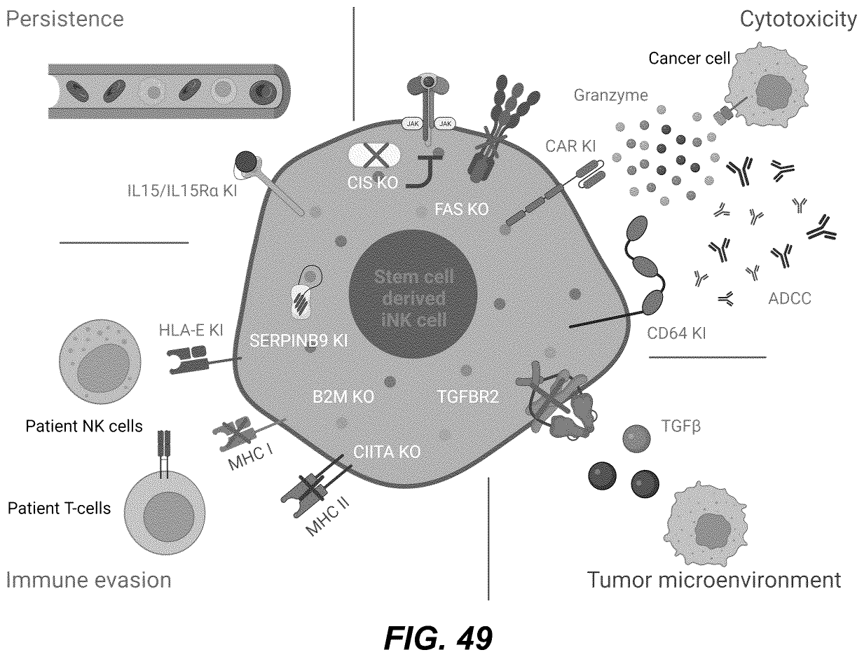

Exemplary edits for engineering an iNK for targeting GPC3 are shown in FIG. 49. Shown below in Table 73 is a non-limiting exemplary summary of isolated iPSC lines with one or more of the edits shown in FIG. 49.

TABLE 73
CELL LINES COMPRISING GPC3 CAR KNOCK-IN

| | | Availability | Characterization | In vitro killing |
|---|---|---|---|---|
| | | of edited | of differentiated | (exemplary target |
| iPSC | Edits* | iPSC cells | iNK cells | cancer cell lines) |
| L7V011 | SerpinB9-IL15/IL15Rα | 8 clones | FACS | K562, HepG2, |
| | KI/B2MKO; GPC3 | | | Hep3B, A498 |
| | CAR KI/CIITA KO | | | |
| L7V013 | CD64KI/CISH KO; | Bulk; 8 clones | | Bulk showed |
| | SerpinB9-IL15/IL15Rα | | | killing of K562 |
| | KI/B2MKO; GPC3 | | | |
| | CAR KI/CIITA KO | | | |
| L7V014 | FAS KO; | | | |
| | CD64KI/CISH KO; | | | |
| | SerpinB9-IL15/IL15Rα | | | |
| | KI/B2MKO; GPC3 | | | |
| | CAR KI/CIITA KO | | | |
| L7V015 | FAS KO; TGFβR2 KO; | 1 clone | | |
| | CD64KI/CISH KO; | | | |
| | SerpinB9-IL15/IL15Rα | | | |
| | KI/B2MKO; GPC3 | | | |
| | CAR KI/CIITA KO | | | |
| L7V018 | IL15/IL15Rα KI/B2M | 8 clones (In | FACS | K562, HepG2, |
| | KO | some embodiments, | | Hep3B, A498 |
| | | as control) | | |
| L7V001 | SerpinB9-IL15/ | 8 clones (In | FACS | K562 |
| | IL15Rα KI/B2MKO | some embodiments, | | |
| | | as control) | | |

*In some embodiments, the cells comprising a CAR KI also comprise KI of an HLA-E trimer.

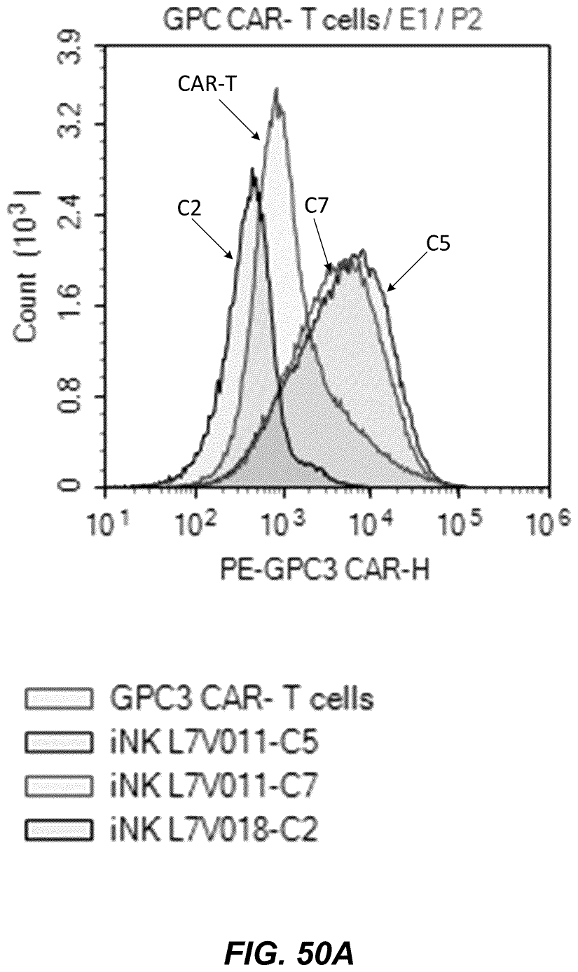

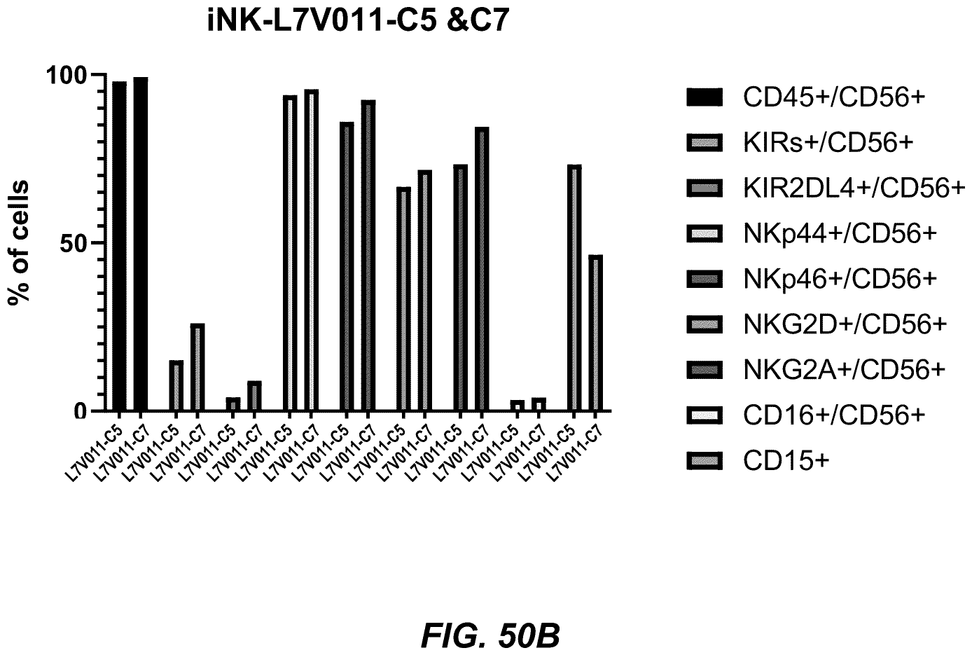

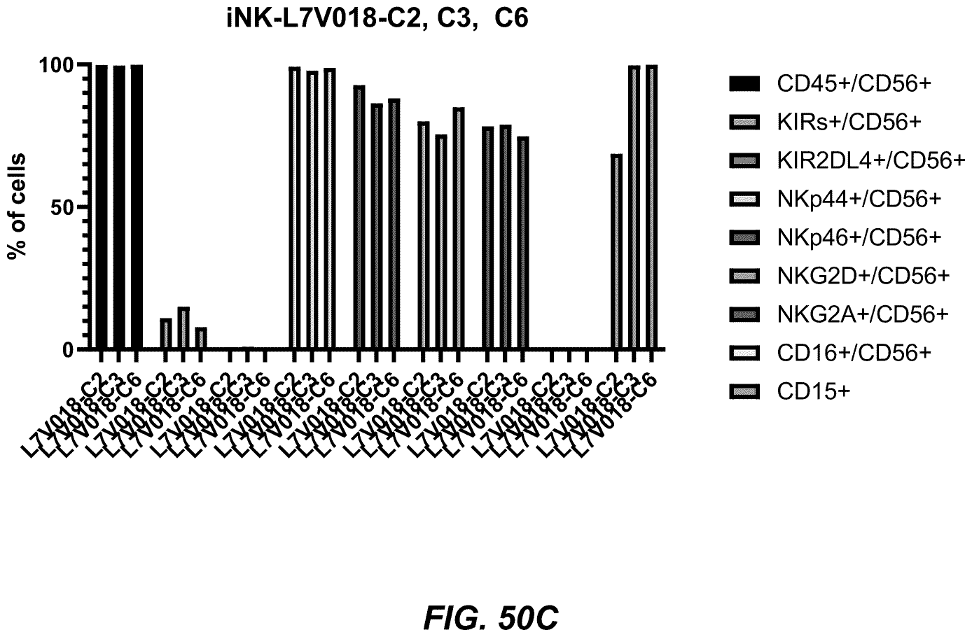

FIG. 50A-FIG. 50C show flow cytometry data characterizing iPS cells that were differentiated into NK cells. Cells with the following edits were analyzed (Also, see Table 73 above): L7V011 edits (SerpinB9-IL15/IL15Rα fusion KI/B2M KO; GPC3 CAR KI/CIITA KO) and L7V018 edits (IL15/IL15Rα fusion KI/B2M KO).

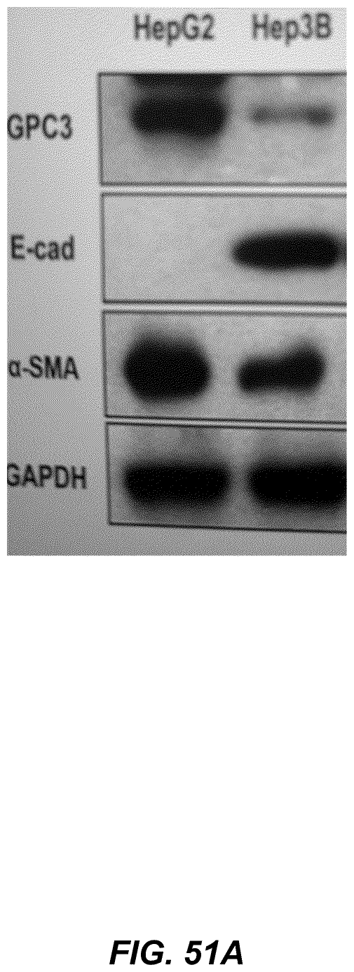

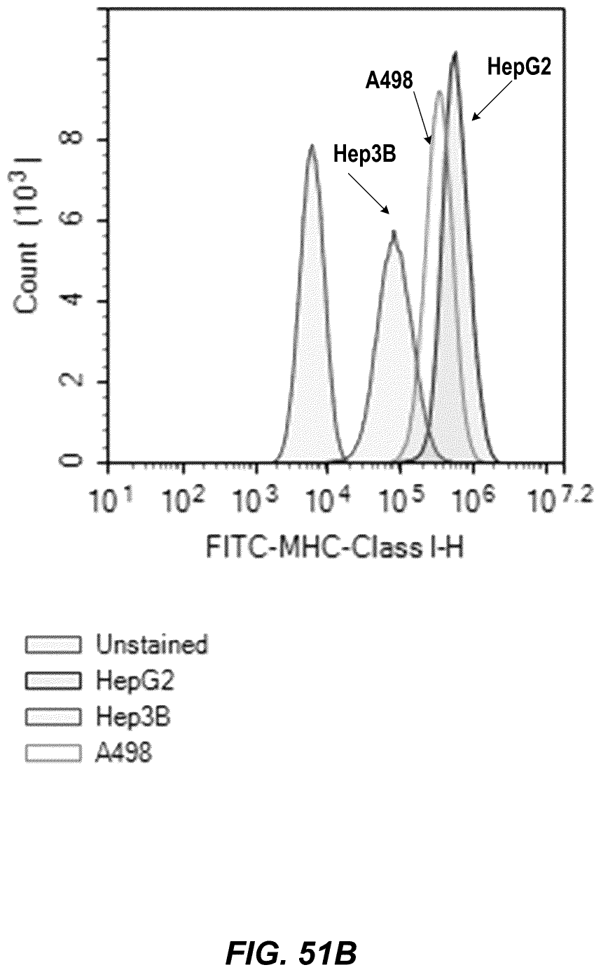

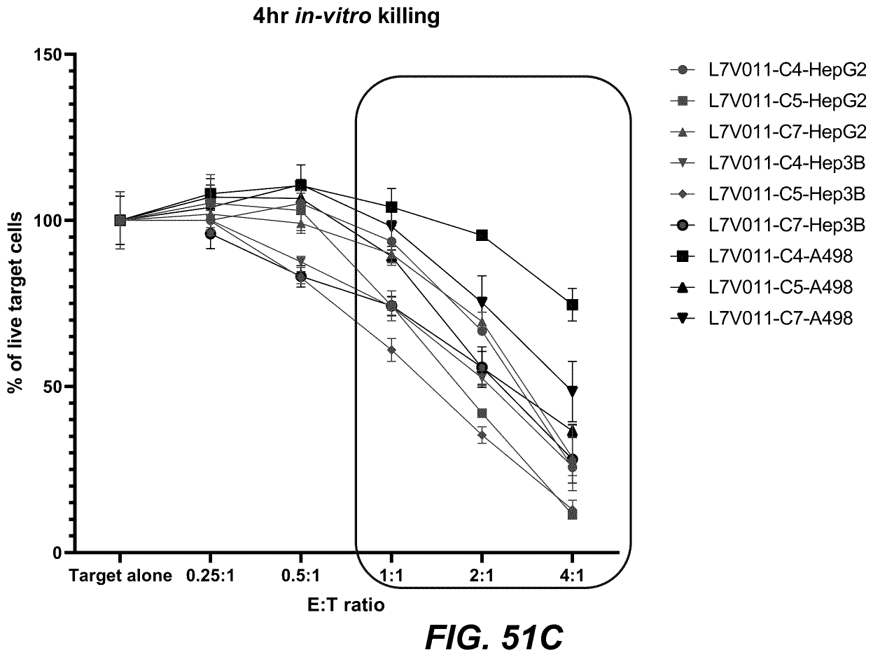

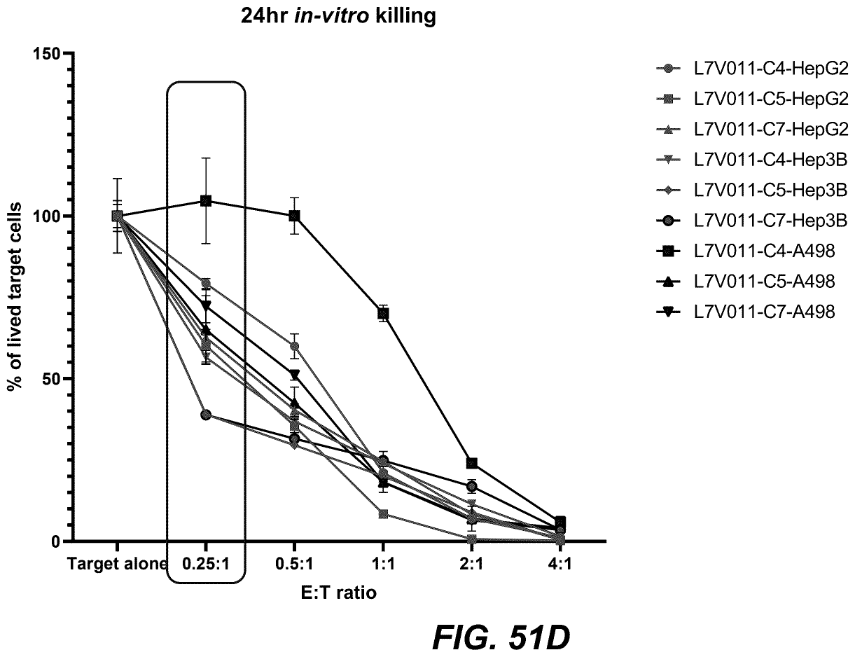

FIG. 51A-FIG. 51D show data related to killing of cancer cells by the anti-GP CAR iNK cells. As provided in FIG. 51A, western blot analysis showed that GPC3 is highly expressed in, e.g., HepG2 cell line as compared to Hep3B. A498 cells, in contrast, are negative for GPC3 expression. These cells express MHC class I on their surface as determined by flow cytometry (FIG. 51B), with highest expression in HepG2 cells and A498 cells, and Hep3B cells displaying medium expression. FIG. 51C-FIG. 51D show killing activity of the iNK cells following 4 hr (FIG. 51C) or 24 hr (FIG. 51D) of co-culture with target cells. The killing efficiency was greatest for Hep3B cells, followed by HepG2, and then A498. Therefore, the GPC3 CAR iNK cells showed specificity against GPC3+ cells.

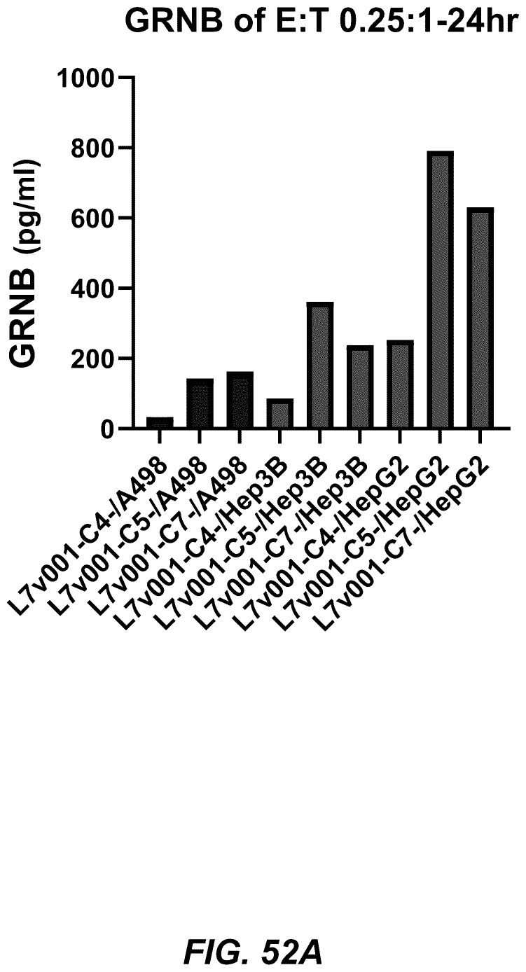

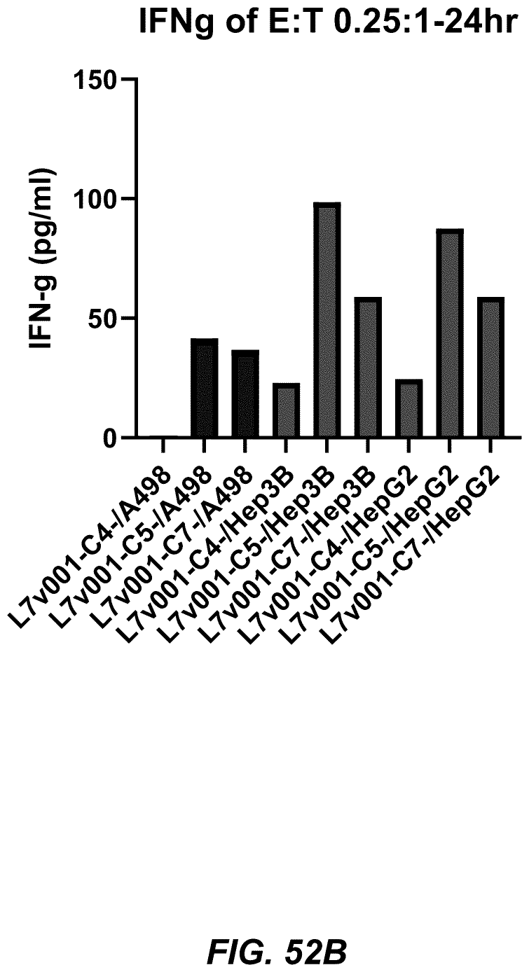

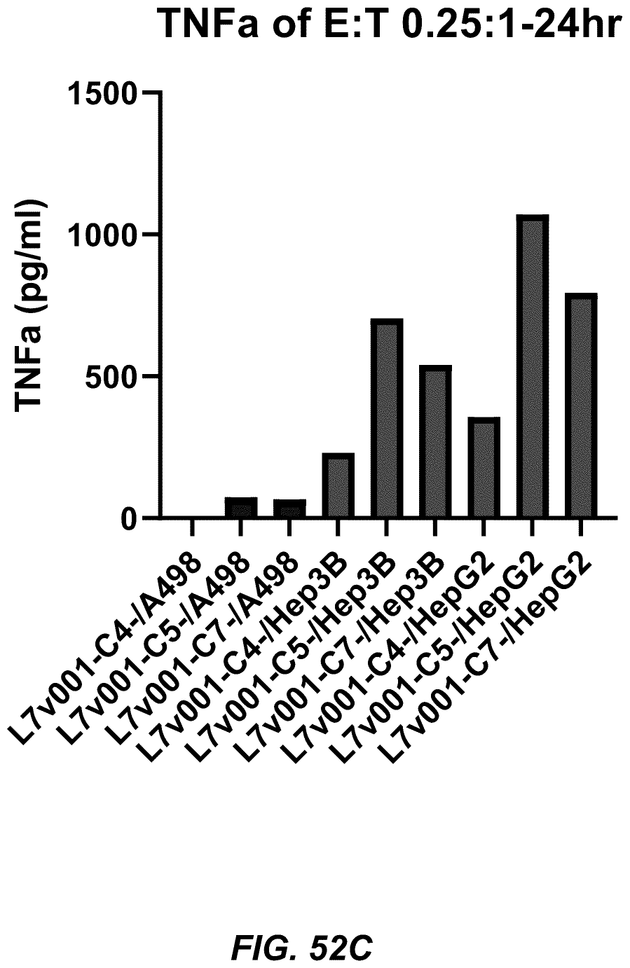

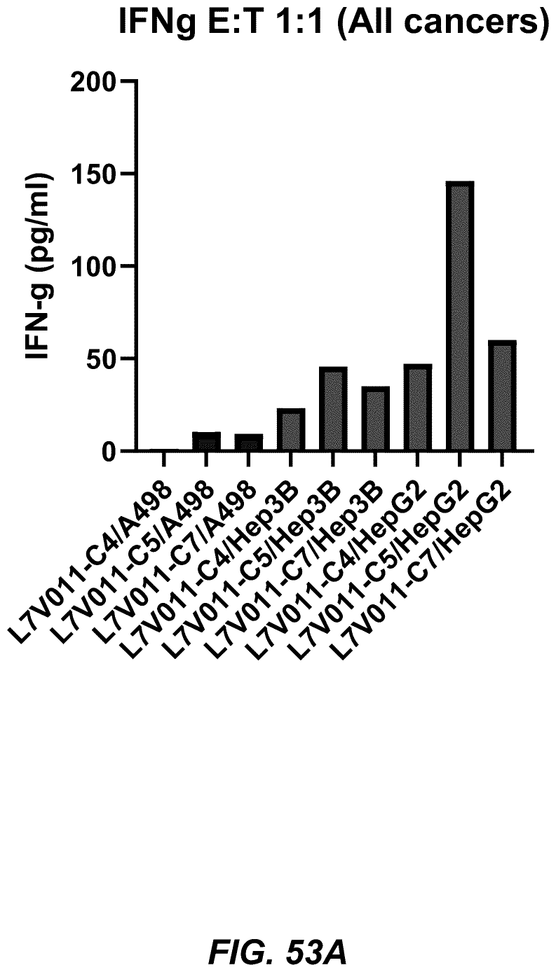

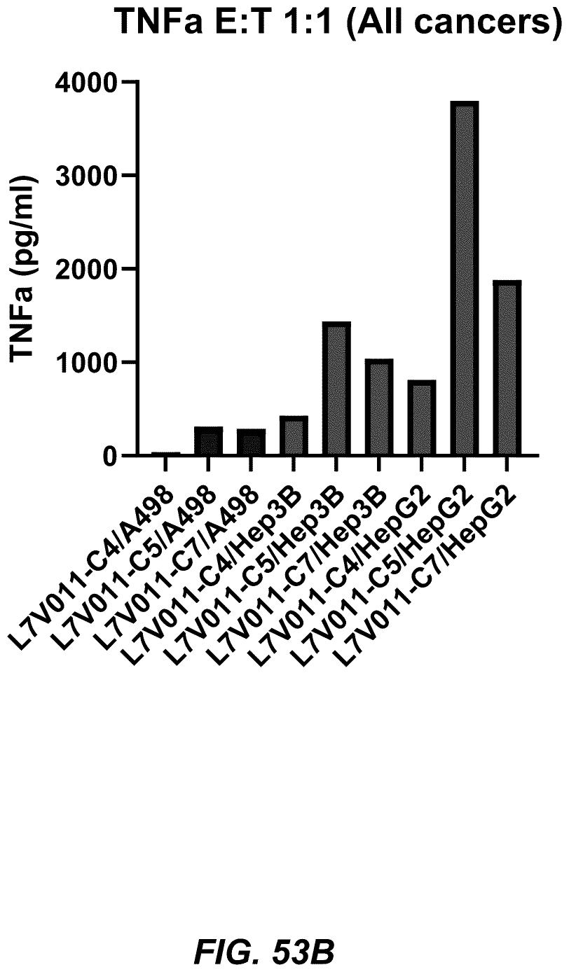

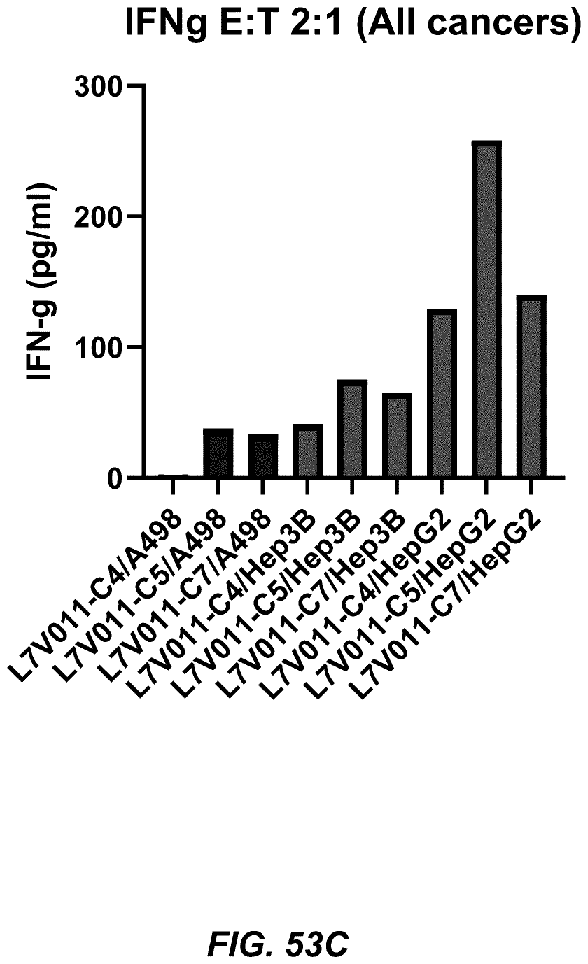

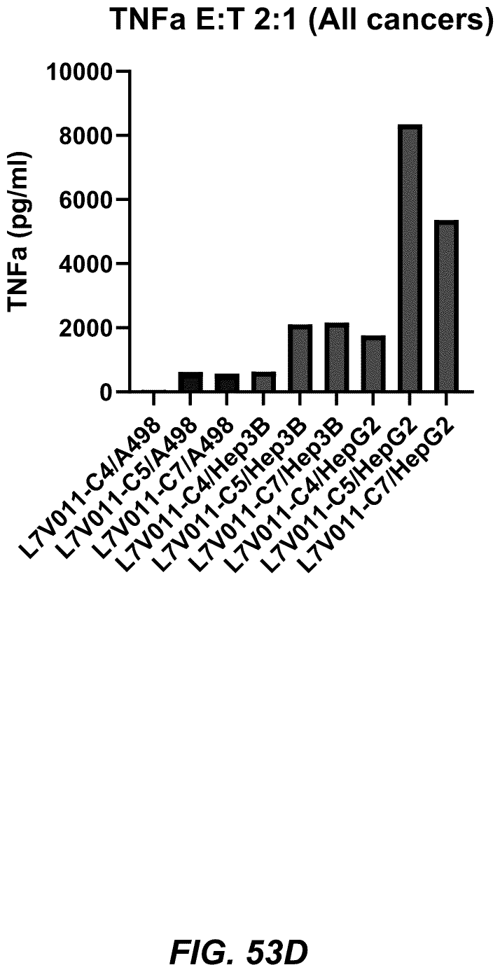

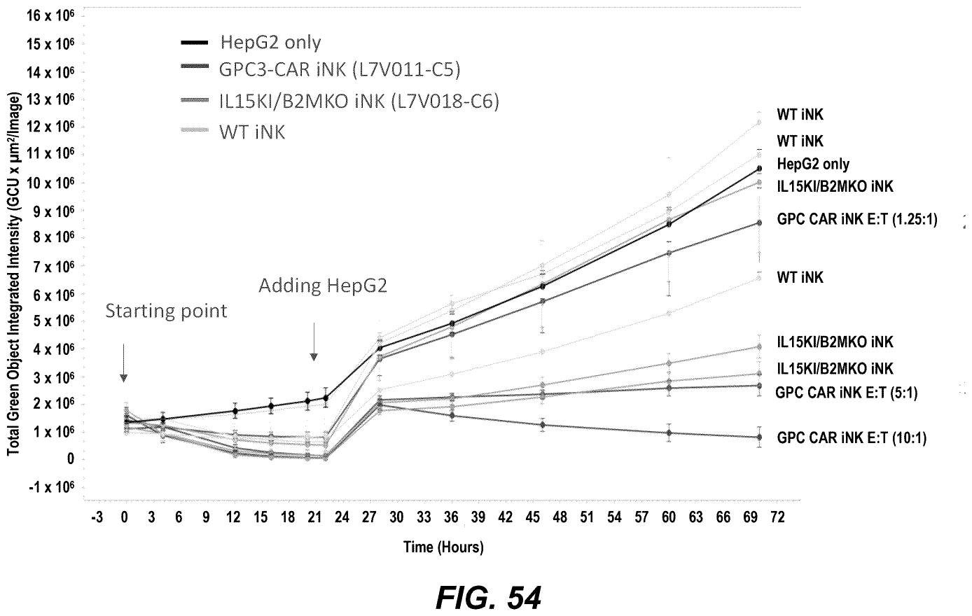

Co-culture of iNK cells with target cells also results in detectable secretion of cytokines. FIG. 52A-FIG. 52C shows detection of Granzyme B (GRNB, FIG. 52A), interferon γ (IFNg, FIG. 52B), and tumor necrosis factor α (TNFa, FIG. 52C) after 24 hr. FIG. 53A-FIG. 53B display levels of cytokines IFNg or TNFa after 4 hrs of co-culture at 1:1 effector to target ratio. FIG. 53C-FIG. 53D display levels of IFNg or TNFa after 4 hrs of co-culture at a 2:1 effector to target ratio. As shown in FIG. 54, the iNK cells targeting GPC3 as disclosed herein are capable of killing target cells (e.g., HepG2) after re-challenge.

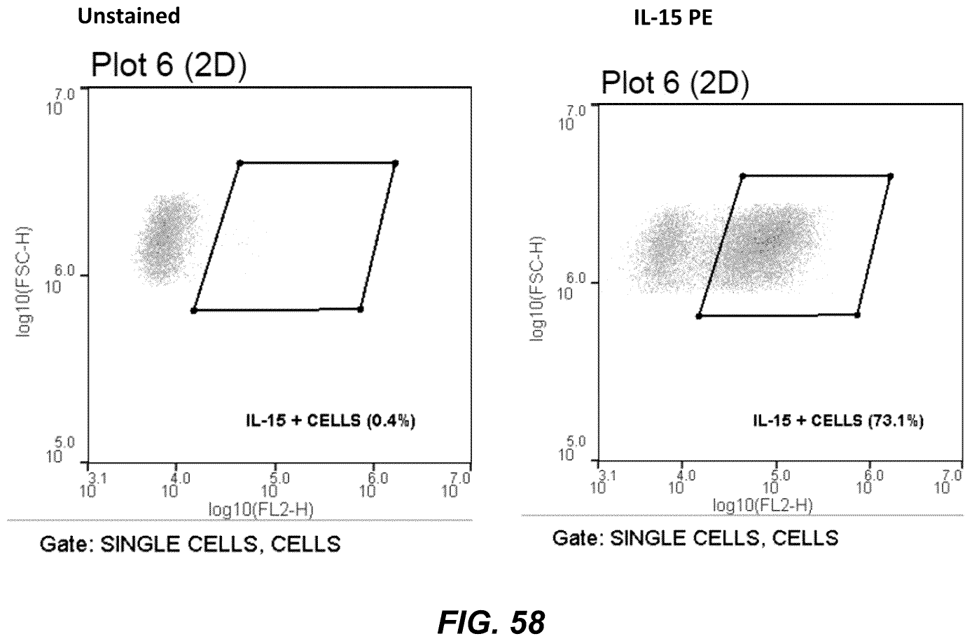

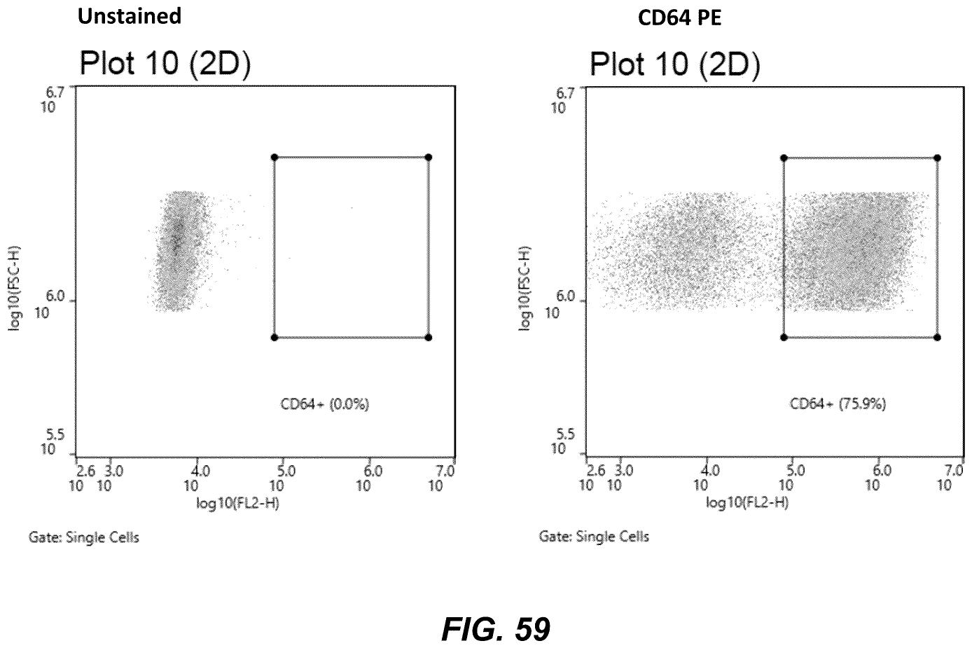

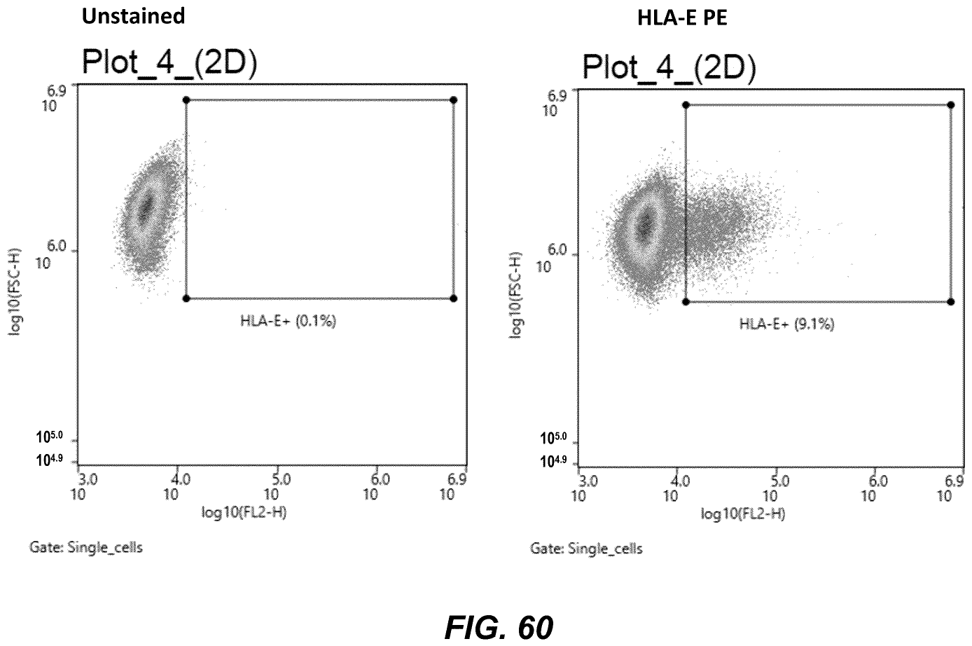

Edited cells can be identified as expressing proteins expressed from knock-in nucleic acid sequences. FIG. 58 shows FACS plots generated during the single cell sorting of the B2M KO-SerpinB9-P2A-IL15/IR15α KI bulk population previously enriched by FACS. This population was 73% positive for IL15 and cells were sorted based on the highest intensity of expression. FIG. 59 shows the FACS plots generated during the single cell sorting of the CISH KO-CD64 KI bulk population previously enriched by FACS. This population was 76% positive for CD64 and cells were sorted based on the highest intensity of expression. FIG. 60 shows the FACS plots generated during the single cell sorting of the CIITA KO-GPC3 CAR-P2A-HLA-E KI bulk population previously enriched by FACS. This population was 9% positive for HLA-E and cells were sorted based on the highest intensity of expression.

Shown in Tables 74-75 are analyses of, e.g., editing in different clones of cell lines L7V015 (comprising SerpinB9-IL15/IL15Rα KI/B2M KO; anti-GPC3 CAR-HLA-E KI/CIITA KO; CD64 KI/CISH KO; TGFβR2 KO; FAS KO, also See Table 73) or L7V030 (comprising SerpinB9-IL15/IL15Rα KI/B2M KO; anti-GPC3 CAR-HLA-E KI/CIITA KO; CD64 KI/CISH KO; FAS KO).

TABLE 74A
L7V015 CLONE ANALYSIS

| | Zygosity | | Zygosity | | Zygosity |
|---|---|---|---|---|---|
| Clone | IL-15 at | INDEL | HLA-E at | INDEL | CD64 |
| name | B2M | B2M | CIITA | CIITA | at CISH |
| L7V015C1 | HET | +1 | Hom | N/A | Hom |
| L7V015C2 | HET | +1 | Hom | N/A | Het |
| L7V015C3 | HET | +1 | Het | −536 bp | Het |
| L7V015C4 | HET | +1 | Het | −536 bp | Hom |
| L7V015C5 | HET | +1 | Het | −536 bp | Hom |
| L7V015C6 | HET | +1 | Het | −536 bp | Het |
| L7V015C7 | HET | +1 | Het | −536 bp | Het |
| L7V015C8 | HET | +1 | Het | −536 bp | Hom |
| L7V015C9 | HET | +1 | Het | −536 bp | Het |
| L7V015C10 | HET | +1 | Het | −536 bp | Het |

10 clones

TABLE 74B
L7V015 CLONE ANALYSIS

| Clone | INDEL | Zygosity | INDEL | Zygosity | INDEL |
|---|---|---|---|---|---|
| name | CIITA | FAS KO | FAS | TGFBR2 KO | TGFBR2 |
| L7V015C1 | N/A | Hom | +1 | Hom | −1/−4 |
| L7V015C2 | +2 | Hom | +1 | Hom | −8 |
| L7V015C3 | +2 | Hom | +1, −4 | Hom | −1 |
| L7V015C4 | N/A | Hom | +1 | Hom | −13 |
| L7V015C5 | N/A | Hom | +1 | Hom | −31 |
| L7V015C6 | −8 | Hom | +1 | Het | −19, +24 |
| L7V015C7 | −8 | Hom | +1 | Hom | −13 |
| L7V015C8 | N/A | Hom | −1 | Hom | −1 |
| L7V015C9 | −8 | Hom | −1, 1 | Hom | −1/−8 |
| L7V015C10 | −8 | Hom | +1 | Hom | −1 |

10 clones

TABLE 75
L7V030 CLONE ANALYSIS

| | Zygosity | | Zygosity | | Zygosity | | | |
|---|---|---|---|---|---|---|---|---|
| Clone | IL-15 at | INDEL | HLA-E at | INDEL | CD64 at | INDEL | Zygosity | INDEL |
| name | B2M | B2M | CIITA | CIITA | CISH | CIITA | FAS KO | FAS |
| L7V030C1 | HET | +1 | HOM | N/A | HOM | N/A | Hom | +1 |
| L7V030C2 | HET | +1 | HOM | N/A | HOM | N/A | Hom | +1 |
| L7V030C3 | HET | −1 | HET | −1 | HOM | −1 | Hom | +1, −4 |
| L7V030C4 | HET | +1 | HET | +1 | HOM | +1 | Hom | +1 |
| L7V030C5 | HET | +1 | HOM | N/A | HOM | N/A | Hom | +1 |
| L7V030C6 | HET | −1 | HOM | N/A | HOM | N/A | Hom | +1 |
| L7V030C7 | HET | +1 | HET | −10 | HOM | −10 | Hom | +1 |
| L7V030C8 | HET | +1 | HOM | N/A | HOM | N/A | Hom | −1 |
| L7V030C9 | HET | −1 | HET | −1 | HOM | −1 | Hom | −1, 1 |
| L7V030C10 | HET | −1 | HET | −1 | HET | −1 | Hom | +1 |

10 clones

### Example 36

### Differentiation and Activity of iNK Cells Targeting GPR87

This example describes the generation and characterization of iNK cells targeting GPR87. iPSCs comprising L7V031 edits (which include, SerpinB9-IL15/IL15Rα KI/B2M KO; GPR87 CAR-HLA-E trimer KI/CIITA KO; CD64 KI/CISH KO); clones 1-10 are analyzed. L7V001-F8 cells as described above in Example 35 (comprising Serpin B9-IL15/IL15Rα KI/B2M KO) were used as a control.

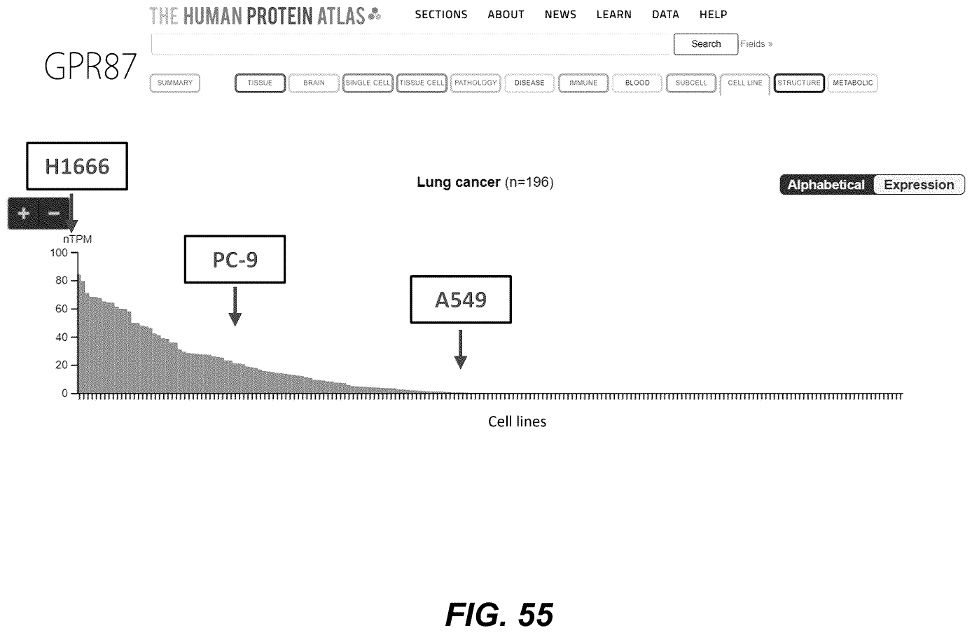

GPR87 is a member of the cell surface molecular G protein-coupled receptors (GPCR) family and, without being bound by any particular theory, contributes to the viability of human tumor cells. Its tumor-specific expression and cell surface location make it an attractive molecule for targeted therapy. 51.2% (63/123) of non-small cell lung cancer (NSCLC) tumors were GPR87-positive and the overexpression of GPR87 correlates with the worse tumor differentiation and high tumor proliferation in NSCLC. The GPR87-overexpressing cells were more frequently observed in lung cancer cells (70%, 7/10). As shown in FIG. 55, expression data from the human protein atlas shows that lung cancer cell lines such as H1666 and PC-9 exhibit high expression of GPR87. The normalized gene expression values (as normalized Transcripts Per Kilobase Million, nTPM) are: H1666, 84.1; PC-9, 17.9; and A549, 0.3. In some embodiments, GPR87 promotes metastasis through the AKT-eNOS-NO axis in lung adenocarcinoma.

Cells comprising SERPINB9-P2A-IL15/IR15α KI and B2M KO, CD64 KI and CISH KO and FAS KO were enriched for said edits. This population was expanded and the cells were electroporated with a plasmid encoding anti-GPR87 CAR-P2A-HLA-E KI and RNP targeting CIITA. This electroporation for KI was done the same way as the electroporation for KI of SERPINB9-P2A-IL15/IR15α above (See, e.g., Example 23). At 2 days post electroporation, the cells were enriched for transfection by performing FACS for HLA-E. At 7 to 10 days post electroporation, the cells were FACS sorted again for HLA-E to enrich for knock in positive cells. After this last targeting, the population represents an enriched bulk of SERPINB9-P2A-IL15/IR15α KI, CD64 KI and anti-GPR87 CAR-P2A-HLA-E KI triple positive cells with a KO of B2M, CISH, FAS and CIITA. To create single cell clone, cells were FACS sorted for HLA-E into a 96 well plate. After 12-14 days, sorted single cell clones were genotyped by PCR. Information on single clones obtained is in Table 76. The cells were differentiated essentially as described in Example 18 and characterized.

TABLE 76
INFORMATION OF SERPINB9-P2A-IL15/IL15RA FUSION KNOCK-IN AND
B2M KNOCK-OUT, ANTI-GPR87 CAR-P2A-HLA-E TRIMER KNOCK-IN AND CIITA
KNOCK-OUT, CD64 KI AND CISH KNOCK-OUT AND FAS KNOCK-OUT

| | Zygosity of | Indel | Zygosity | Indel | Zygosity | Indel | Zygosity | Indel |
|---|---|---|---|---|---|---|---|---|
| Cell Line | KI | Analysis | of KI | Analysis | of KI | Analysis | of KO | Analysis |
| Name | B2M | B2M | CIITA | CIITA | CISH | CISH | FAS | FAS |
| L7V031C1 | Het | +1 | Het | −2 | Hom | N/A | Hom | +1 |
| L7V031C2 | Het | −1 | Hom | N/A | Het | +1 | Hom | +1 |
| L7V031C3 | Het | −34 | Het | −6 | Hom | N/A | Hom | +1, +2 |
| L7V031C4 | Het | −34 | Hom | N/A | Hom | N/A | Hom | +1, −22 |
| L7V031C5 | Het | +1 | Het | −2 | Hom | N/A | Hom | +1, −40 |
| L7V031C6 | Het | −1 | Hom | N/A | Hom | N/A | Hom | +1 |
| L7V031C7 | Het | −34 | Het | −12 | Hom | N/A | Hom | +1 |
| L7V031C8 | Het | −1 | Het | −28 | Hom | N/A | Hom | +1 |
| L7V031C9 | Het | −1 | Het | −28 | Hom | N/A | Hom | +1 |
| L7V031C10 | Hom | N/A | Het | +1 | Het | −4 | Hom | +1 |

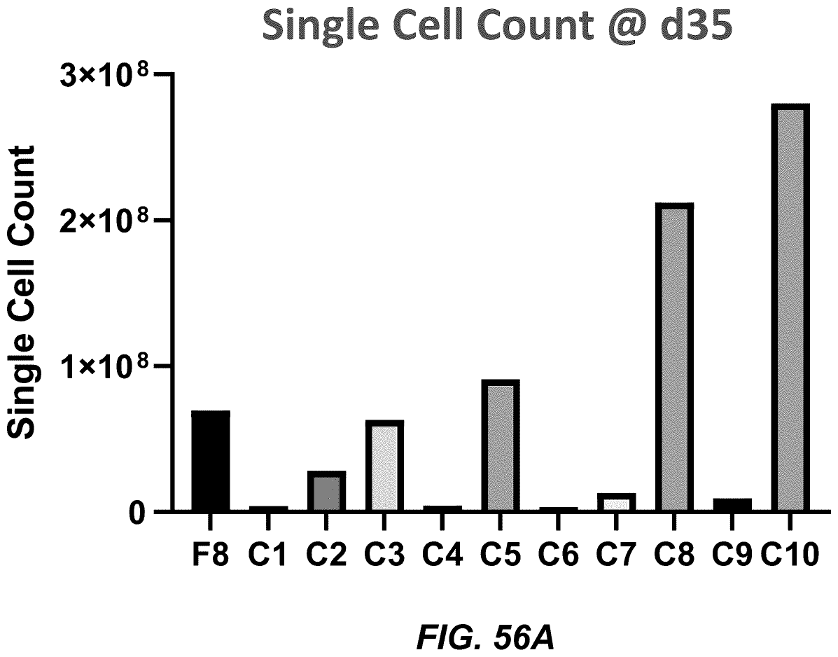

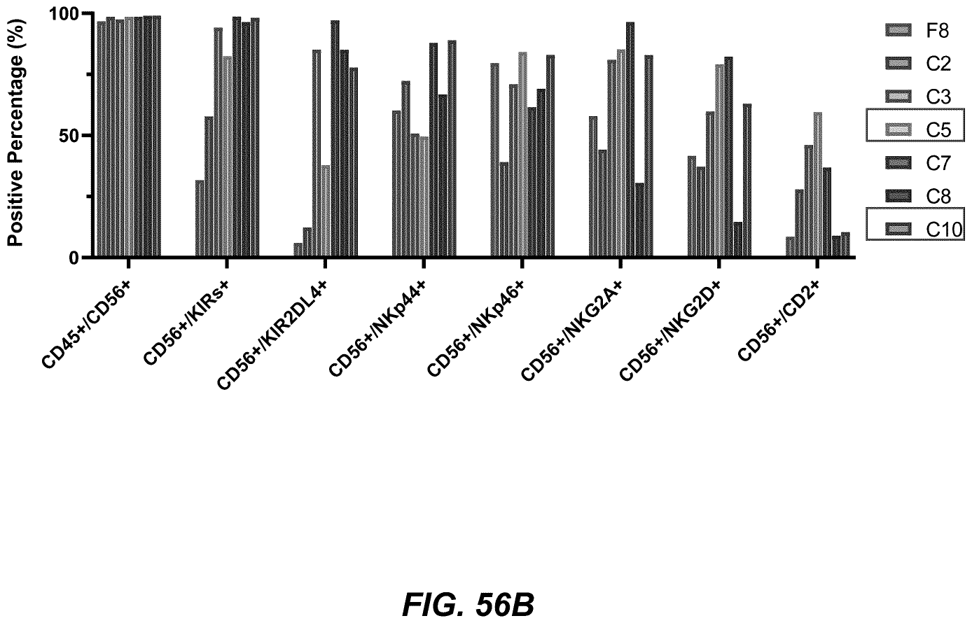

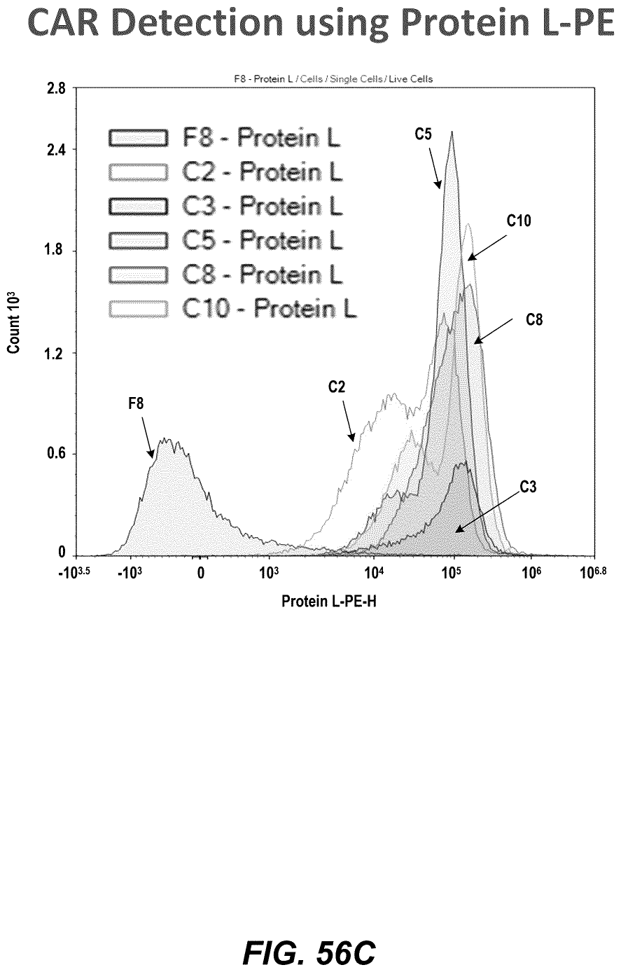

FIG. 56A-FIG. 56C display cell count and FACS data of iNK cells differentiated from engineered iPSCs. Out of 10 clones, 5 clones (C2, C3, C5, C8, C10) had high single cell release at d35 (FIG. 56A). The top three clones were C10, C8 and C5, while C10 showed >280-fold expansion. As shown in FIG. 56B, C8 had relatively low percentage of NKG2A+ and NKG2D+ compared to C5 and C10, while C5 had high percentage of CD2+, which can potentially increase NK cell in vivo persistence. All clones had high Protein L labeling (FIG. 56C). Protein L properties are described below in Table 77.

TABLE 77
BACKGROUND OF PROTEIN L

| Reagents | Mechanism | Pros | Cons |
|---|---|---|---|
| Target | Specifically bind to | High specificity | High cost |
| Antigens | the antigen-binding | Minimal | Each unique CAR has to be |
| | domains of CARs. | background | stained with corresponding |
| | | staining | antigens |
| Protein L | Binds to the kappa | Universal and | High background staining. |
| | light chain of | low cost | Cannot detect the anti-lambda |
| | immunoglobulin. | | light chain CAR |
| Anti-Fab | Binds to the Fab | Universal and | High background staining |
| antibody | portion of | low cost | |
| | immunoglobulin. | | |
| Anti- | Specifically bind to | High specificity; | Each unique CAR has to be |
| idiotypic | the antigen-binding | High sensitivity | stained with corresponding Anti- |
| Antibody | domains of CARs. | Minimal | idiotypic Antibody. |
| | | background | Most need to be customized and |
| | | staining. | customization is very time- |
| | | | consuming |

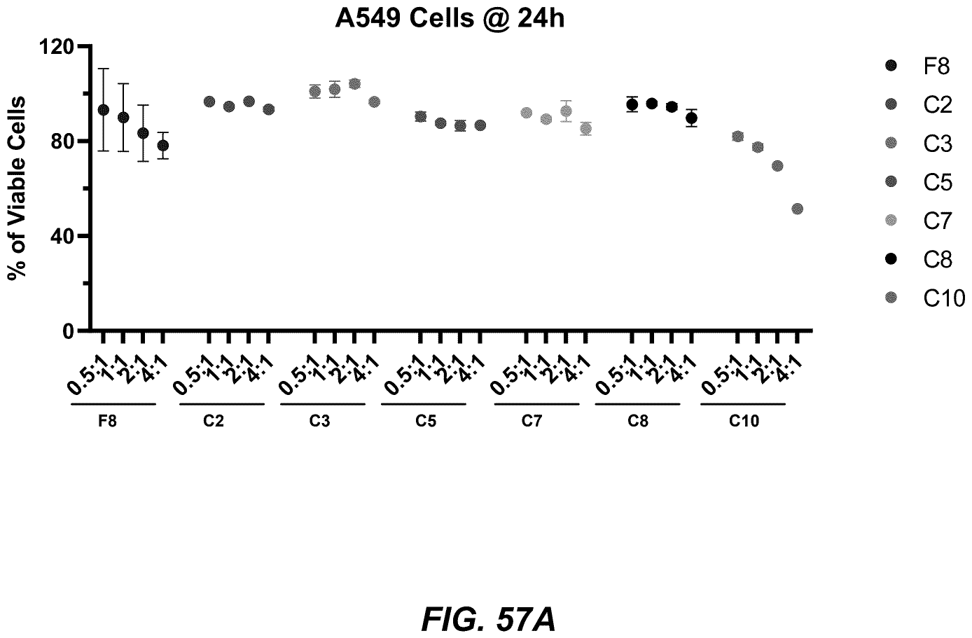

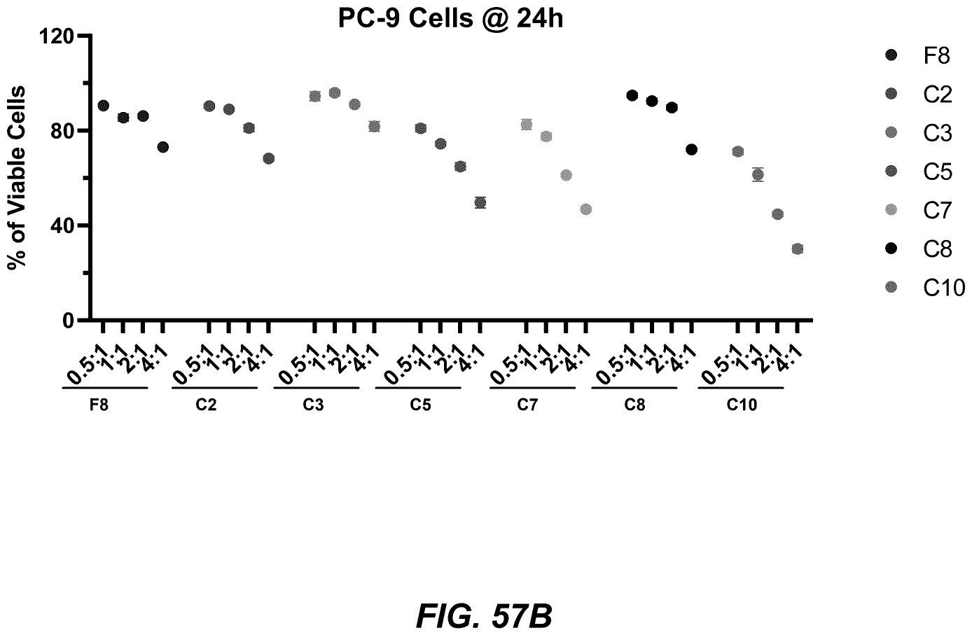

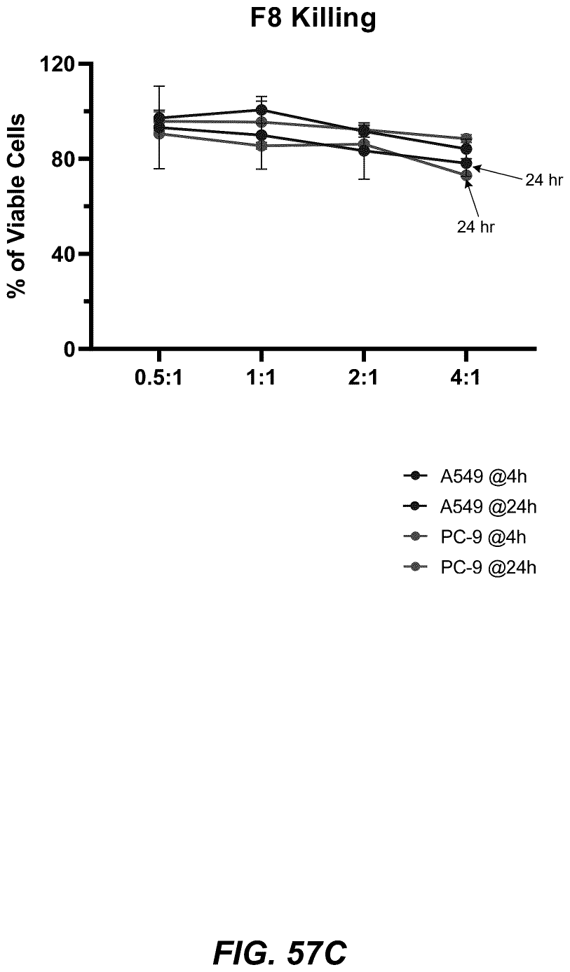

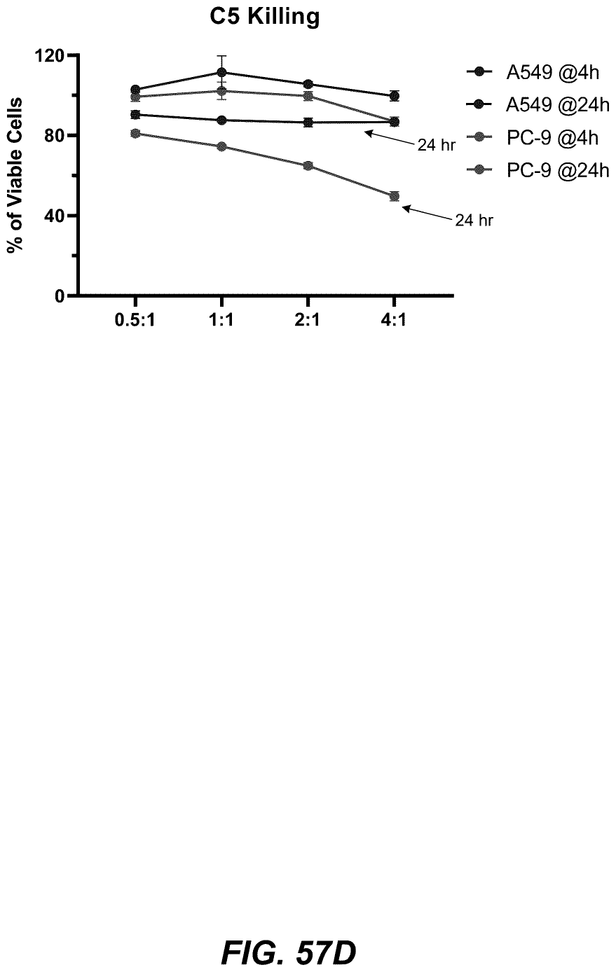

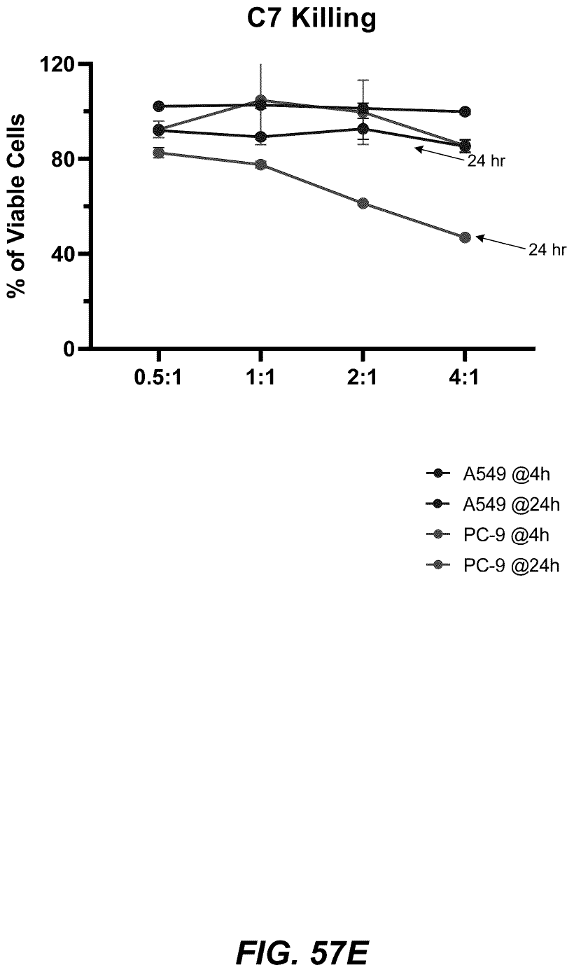

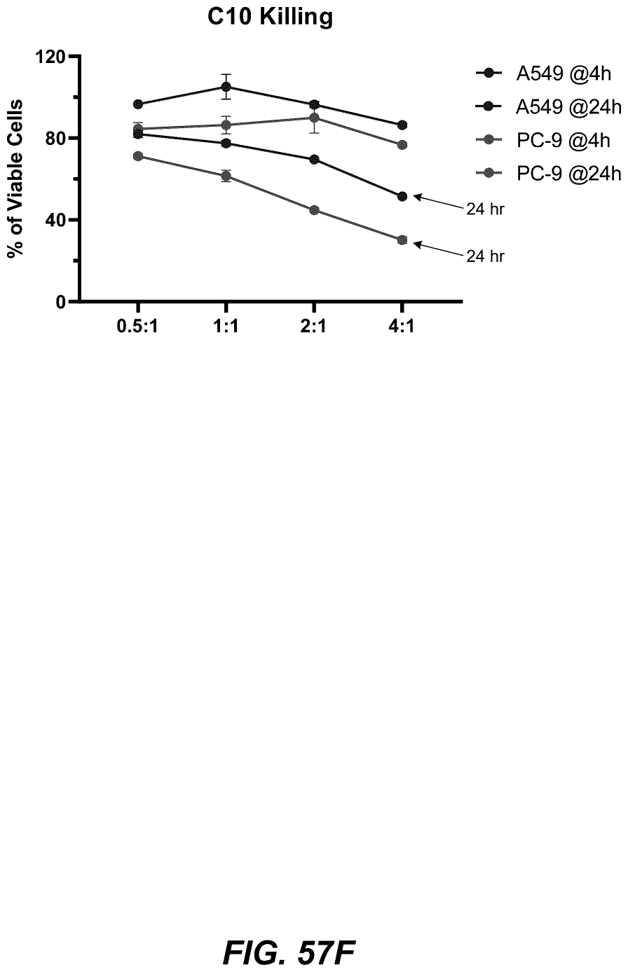

Killing activity of the GPR87 CAR iNKs are shown in FIG. 57A-FIG. 57F. All clones except C10 showed much less cell killing (<20%) against A549 (low GPR87 expression) than PC-9 cells. For PC-9 (high GPR87 expression) cancer cells, the killing effect of C10 was greater than C5 and C7, which were similar to each other. The low cytotoxicity of C8 may, in some embodiments, be due to its low expression of NKG2A and NKG2D. L7V001-F8 (without GPR87 CAR) showed similar killing effect against both A549 and PC-9 (<20%).

In summary, GPR87 CAR contributes to the specific killing against GPR87 over-expressing lines. In cells without GPR87 CAR, no difference in killing against A549 and PC-9 was observed. With GPR87 CAR, less killing against GPR87 low-expressing line (A549), and high killing against GPR87 over-expressing line (PC-9) was observed.

Shown in Table 78 below are some of the elements of a donor plasmid for knock-in of anti-GPR87-CAR and HLA-E into CIITA gene locus.

TABLE 78
ELEMENTS OF (CIITA) ANTI-GPR87
CAR-P2A-HLA-E DONOR PLASMID

| Element | Location (size in bp) | SEQ ID NO: |
|---|---|---|
| Left HA | 11,175-766 (800) | 22 |
| CMV enhancer | 795-1174 (380) | 23 |
| chicken β-actin promoter | 1177-1452 (276) | 24 |
| Chimeric intron | 1453-2461 (1009) | 25 |
| GPR87 CAR | 2506-3960 (1455) | 159 |
| Signal peptide | 2506-2568 (63) | 160 |
| CD8 hinge | 3286-3420 (135) | 161 |
| CD28 TM | 3421-3624 (204) | 162 |
| Cd3z | 3625-3960 (336) | 163 |
| GSG tag | 3961-3969 (9) | N.A. |
| P2A | 3970-4026 (57) | 45 |
| B2M signal sequence | 4027-4086 (60) | 46 |
| HLA-G peptide | 4087-4113 (27) | 47 |
| GS Linker | 4114-4158 (45) | 48 |
| B2M | 4159-4455 (297) | 49 |
| GS Linker | 4456-4515 (60) | 50 |
| HLA-E | 4516-5526 (1011) | 51 |
| Entire plasmid | 11,208 | 158 |

### Example 37

### In Vivo Analysis of Efficacy and Persistence of Anti-GPR87 iNK Cells

Described in this Example are in vivo studies of iNK cells of the disclosure.

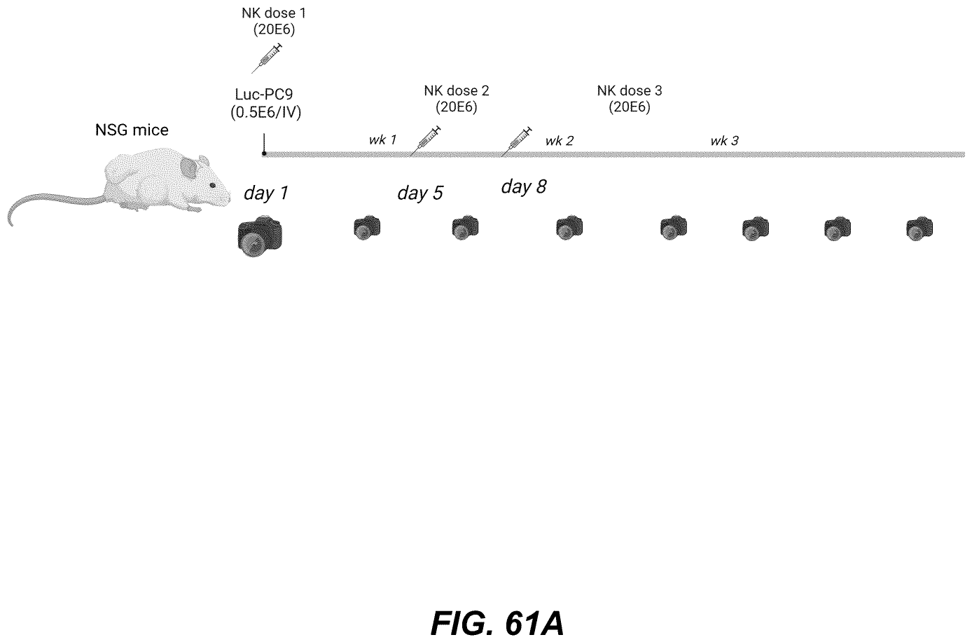

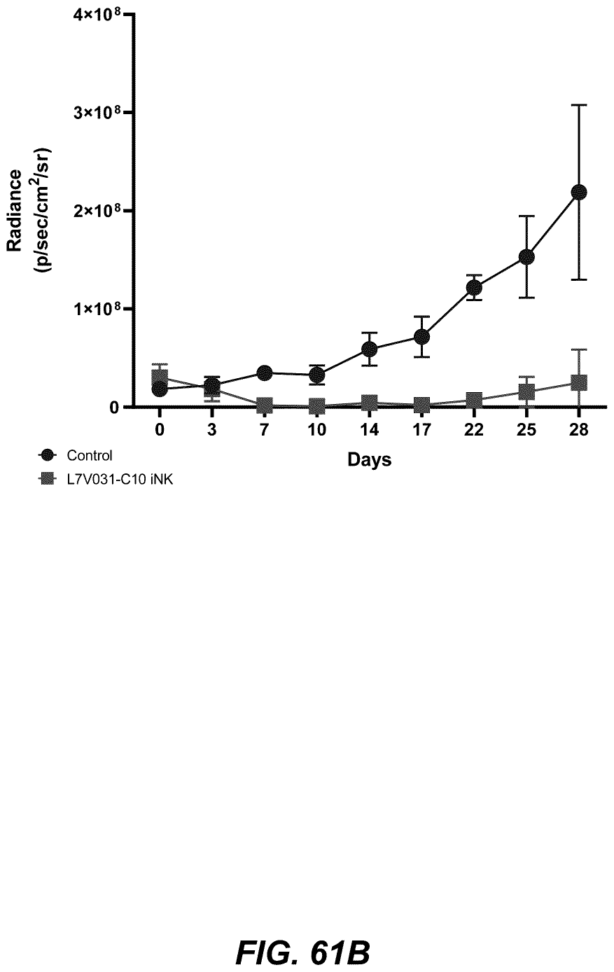

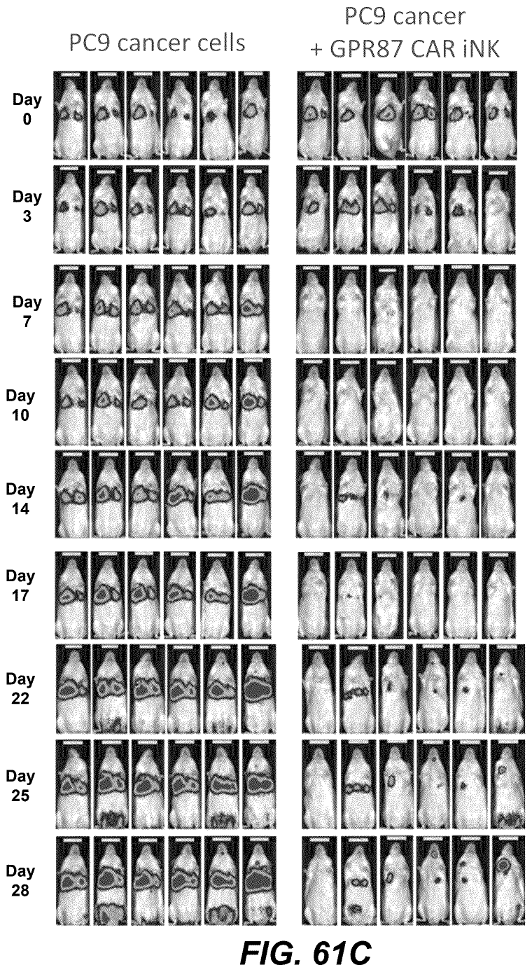

As shown in FIG. 61A, NSG immunodeficient mice were injected with about 0.5×10⁶ PC-9 lung cancer cells that also express luciferase. Mice were also injected with a dose of iNK cells comprising the edits shown below in Table 79. Animals were dosed with iNK cells 3 times over about a week.

TABLE 79
iNK LINES USED FOR IN VIVO EXPERIMENTS

| Exp- | | | | |
|---|---|---|---|---|
| PP#12 | iNK cells | Animal# | Edits | |
| Gr#2 | PT-L7V031- | 6 | CD64 KI/CISH KO; | *animals |
| | C5 | | SerpinB9/IL15/IL15Rα KI/B2M | were dead |
| | | | KO; GPR87 CAR-HLA-E KI/CIITA | after iNK |
| | | | KO | injection |
| Gr#3 | PT-L7V031- | 6 | CD64 KI/CISH KO; SerpinB9/ | |
| | C10 | | IL15/IL15Rα KI/B2M KO; GPR87 | |
| | | | CAR-HLA-E KI/CIITA KO | |

Animals injected with cells derived from clone 10 were monitored for efficacy in preventing growth of cancer in vivo. As shown in FIG. 61B-FIG. 61C, the anti-GPR87 iNK almost completely inhibited growth of the PC9 cancer cells for a month after administration.

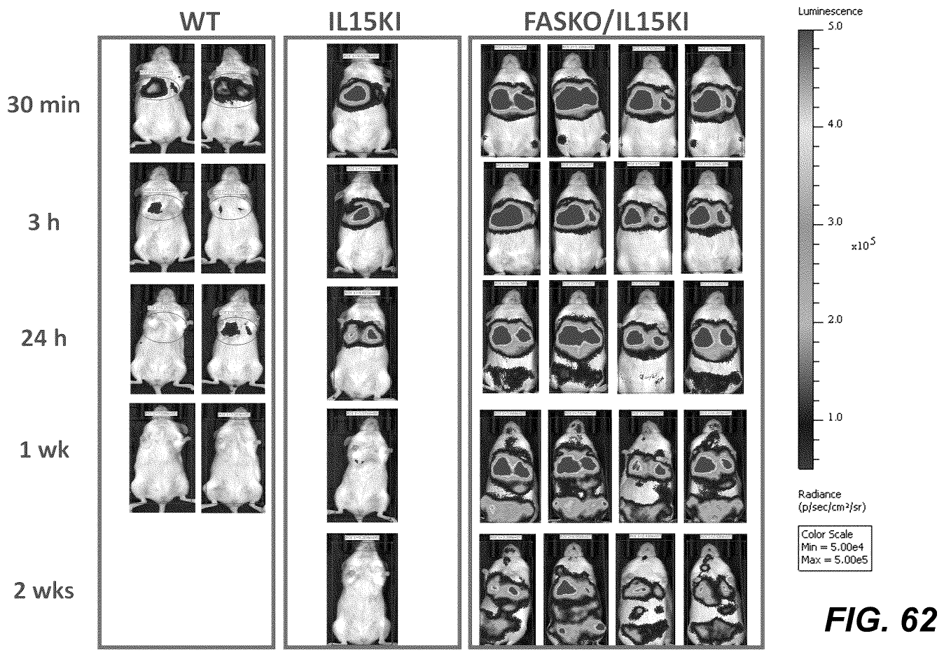

FIG. 62 shows images of iNK cells that comprise the IL15/IL15Rα KI with or without FAS KO (compared to wild-type, e.g., unedited cells). As can be seen from the images, iNK cells were distributed to lung first after iv injection and then gradually distributed out.

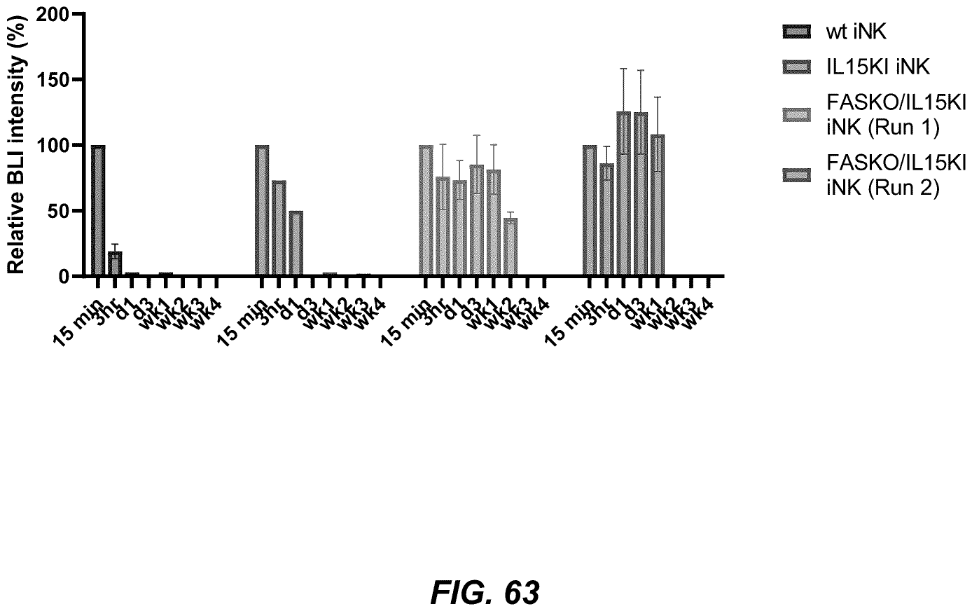

FIG. 63 shows that most WT-iNK cells were dead in 3 hr post-injection and IL15/IL15Rα KI iNK survived in vivo for 24 hr. FAS KO/IL15/IL15Rα iNK cells survived up to 1 week and decreased down to 50% at week 2. These results were reproduced in run 2.

TABLE 80
SEQUENCES

| | | | SEQ |
|---|---|---|---|
| | | | ID |
| Name | Description | Sequence | NO: |
| ADAM17 | gRNA target | GGTCGCGGCGCCAGCACGAA | 1, |
| Ex1_T2 | sequence in | | 165 |
| | ADAM17, spacer | | |
| | sequence | | |
| ADAM17 | gRNA target | CCGAAGCCCGGGTCATCCGG | 2, |
| Ex1_T4 | sequence in | | 166 |
| | ADAM17, spacer | | |
| | sequence | | |
| ADAM17 | gRNA targeti | CCGCGACCTCCGGATGACCC | 3, |
| Ex1_T9 | sequence in | | 167 |
| | ADAM17, spacer | | |
| | sequence | | |
| ADAM17 | gRNA target | CGTGCTGGCGCCGCGACCTC | 4, |
| Ex1_T10 | sequence in | | 168 |
| | ADAM17, spacer | | |
| | sequence | | |
| ADAM17 | gRNA target | CGAAAGGAACCACGCTGGTC | 5, |
| Ex1_T11 | sequence in | | 169 |
| | ADAM17, spacer | | |
| | sequence | | |
| ADAM17 | gRNA target | CAGCGTGGTTCCTTTCGTGC | 6, |
| Ex1_T12 | sequence in | | 170 |
| | ADAM17, spacer | | |
| | sequence | | |
| ADAM17 | gRNA target | GCCGCGACCTCCGGATGACC | 7, |
| Ex1_T19 | sequence in | | 171 |
| | ADAM17, spacer | | |
| | sequence | | |
| ADAM17 | gRNA target | GAACCACGCTGGTCAGGAAT | 8, |
| Ex1_T24 | sequence in | | 172 |
| | ADAM17, spacer | | |
| | sequence | | |
| ADAM17 | gRNA target | CAGCACGAAAGGAACCACGC | 9, |
| Ex1_T25 | sequence in | | 173 |
| | ADAM17, spacer | | |
| | sequence | | |
| ADAM17 | gRNA target | GTAGCGGGGCCGGGAACATG | 10, |
| Ex1_T28 | sequence in | | 174 |
| | ADAM17, spacer | | |
| | sequence | | |
| ADAM17 | Primer for | AGAATCTTCCCAGTAGGCGG | 11 |
| F2 | ADAM17 | | |
| ADAM17 | Primer for | CTCAGGCGCTCAGTCACTAC | 12 |
| R2 | ADAM17 | | |
| CIITA | gRNA target | GGTCCATCTGGTCATAGAAG | 13, |
| Ex3_T6 | sequence in | | 175 |
| | CIITA, spacer | | |
| | sequence | | |
| CIITA | gRNA target | GCTCCAGGTAGCCACCTTCT | 14, |
| Ex3_T16 | sequence in | | 176 |
| | CIITA, spacer | | |
| | sequence | | |
| CIITA | gRNA target | TAGGGGCCCCAACTCCATGG | 15, |
| Ex3_T20 | sequence in | | 177 |
| | CIITA, spacer | | |
| | sequence | | |
| CIITA | gRNA target | GGCTTATGCCAATATCGGTG | 16, |
| Ex4_T1 | sequence in | | 178 |
| | CIITA, spacer | | |
| | sequence | | |
| CIITA | gRNA target | AGGTGATGAAGAGACCAGGG | 17, |
| Ex4_T25 | sequence in | | 179 |
| | CIITA, spacer | | |
| | sequence | | |
| CIITA_F5 | Primer for | TCCTGACTCTCTGGTGTGAGAT | 18 |
| | CIITA | | |
| CIITA_R5 | gRNA target | CAGAGAGCGTCCCACAGAC | 19 |
| | sequence in | | |
| | CIITA | | |
| 86 bp of | 86 base pairs | GCCACCATGGAGTTGGGGCCCCTAGAAGGTGGCTA | 20 |
| CIITA | (bp) of CIITA | CCTGGAGCTTCTTAACAGCGATGCTGACCCCCTGTG | |
| | exon 2 removed | CCTCTACCACTTCTA | |
| | after homology | | |
| | directed repair | | |
| Left ITR | Left inverted | CCTGCAGGCAGCTGCGCGCTCGCTCGCTCACTGAGG | 21 |
| | terminal repeat | CCGCCCGGGCGTCGGGCGACCTTTGGTCGCCCGGCC | |
| | | TCAGTGAGCGAGCGAGCGCGCAGAGAGGGAGTGGC | |
| | | CAACTCCATCACTAGGGGTTCCT | |
| LHA- | Left homology | catatttatggggtatatgtgaatatttattacatgcatagaaggtataatgatcatgtcag | 22 |
| CIITA | arm-CIITA | gatatttgaggtatccacatttgggattgtttaaagattaaatgaaatagtgttaaaagtatt | |
| | | taatatgcccttcaacaaatgatgaggaaatcttagaatctgctcagactccttcagttta | |
| | | catattaggaaactgaggcacagaaaggagcagagacttgctcaagtccacccaaa | |
| | | gcagtagagcattgtggttaaatgcaggacttcagtcagactgtctgggttcaaatcct | |
| | | ggttccacttggacatgggtttccttacataaatcacttcacctctctgagcctcagttttct | |
| | | catatgcaaagtgaggataataataataccttccttacatggttactgatatgagtattaa | |
| | | atgtgccagctcatgtgcctggcgtataggaggtgctttataaaccttagctgttaccac | |
| | | tcatggcattgccaaatgtgggacgggtctcctgactctctggtgtgagattgatggaat | |
| | | ccacactttccagttcccttttctacctcctgggtatcttctcatatggttgtaagttccttgg | |
| | | aggaagggaatgtggcttgctctctccaccacgctgagcatataagaggtgctgaatg | |
| | | agcgcttttattcactcctctcatccccagccctcaccagctgggagttgttgtaggtgtc | |
| | | aattttctgcctctttccaacaccctgtgaggtgactgagcattgtcttccctcccaggca | |
| | | gctcacagtgt | |
| CMV | CMV enhancer | gacattgattattgactagttattaatagtaatcaattacggggtcattagttcatagccca | 23 |
| enhancer | for CIITA | tatatggagttccgcgttacataacttacggtaaatggcccgcctggctgaccgcccaa | |
| | plasmid | cgacccccgcccattgacgtcaataatgacgtatgttcccatagtaacgccaataggg | |
| | | actttccattgacgtcaatgggtggactatttacggtaaactgcccacttggcagtacat | |
| | | caagtgtatcatatgccaagtacgccccctattgacgtcaatgacggtaaatggcccg | |
| | | cctggcattatgcccagtacatgaccttatgggactttcctacttggcagtacatctacgt | |
| | | attagtcatcgctattaccatg | |
| chicken β- | chicken β-actin | tcgaggtgagccccacgttctgcttcactctccccattcccccccctccccaccccca | 24 |
| actin | promoter for | attttgtatttatttattttttaattattttgtgcagcgatgggggcgggggggggggggg | |
| promoter | CIITA plasmid | cgcgcgccaggcggggcggggcggggcgaggggcggggcggggcgaggcgg | |
| | | agaggtgcggcggcagccaatcagagcggcgcgctccgaaagtttccttttatggcg | |
| | | aggcggcggcggcggcggccctataaaaagcgaagcgcgcggcgggcg | |
| chimeric | chimeric intron | ggagtcgctgcgttgccttcgccccgtgccccgctccgcgccgcctcgcgccgccc | 25 |
| intron | for CIITA | gccccggctctgactgaccgcgttactcccacaggtgagcgggcgggacggccctt | |
| | plasmid | ctcctccgggctgtaattagcgcttggtttaatgacggctcgtttcttttctgtggctgcgt | |
| | | gaaagccttaaagggctccgggagggccctttgtgcgggggggagcggctcgggg | |
| | | ggtgcgtgcgtgtgtgtgtgcgtggggagcgccgcgtgcggcccgcgctgcccgg | |
| | | cggctgtgagcgctgcgggcgcggcgcggggctttgtgcgctccgcgtgtgcgcga | |
| | | ggggagcgcggccgggggcggtgccccgcggtgcgggggggctgcgagggga | |
| | | acaaaggctgcgtgcggggtgtgtgcgtgggggggtgagcagggggtgtgggcg | |
| | | cggcggtcgggctgtaacccccccctgcacccccctccccgagttgctgagcacgg | |
| | | cccggcttcgggtgcggggctccgtgcggggcgtggcgcggggctcgccgtgccg | |
| | | ggcggggggtggcggcaggtgggggtgccgggcggggcggggccgcctcggg | |
| | | ccggggagggctcgggggaggggcgcggcggccccggagcgccggcggctgtc | |
| | | gaggcgcggcgagccgcagccattgccttttatggtaatcgtgcgagagggcgcag | |
| | | ggacttcctttgtcccaaatctggcggagccgaaatctgggaggcgccgccgcaccc | |
| | | cctctagcgggcgcgggcgaagcggtgcggcgccggcaggaaggaaatgggcg | |
| | | gggagggccttcgtgcgtcgccgcgccgccgtccccttctccatctccagcctcggg | |
| | | gctgccgcagggggacggctgccttcgggggggacggggcagggcggggttcgg | |
| | | cttctggcgtgtgaccggcggctctagagcctctgctaaccatgttcatgccttcttctttt | |
| | | tcctacag | |
| Signal | Signal peptide | atggcgcttccggtgacagcactgctcctccccttggcgctgttgctccacgcagcaa | 26 |
| peptide | sequence of | ggccg | |
| sequence of | human CD8a | | |
| human | | | |
| CD8a | | | |
| BCMA | BCMA targeting | caggtgcagctggtgcagagcggagccgagctcaagaagcccggagcctccgtga | 27 |
| targeting | fragment | aggtgagctgcaaggccagcggcaacaccctgaccaactacgtgatccactgggtg | |
| fragment | | agacaagcccccggccaaaggctggagtggatgggctacatcctgccctacaacga | |
| | | cctgaccaagtacagccagaagttccagggcagggtgaccatcaccagggataaga | |
| | | gcgcctccaccgcctatatggagctgagcagcctgaggagcgaggacaccgctgtg | |
| | | tactactgtacaaggtgggactgggacggcttctttgacccctggggccagggcaca | |
| | | acagtgaccgtcagcagcggcggcggaggcagcggcggcggcggcagcggcgg | |
| | | aggcggaagcgaaatcgtgatgacccagagccccgccacactgagcgtgagccct | |
| | | ggcgagagggccagcatctcctgcagggctagccaaagcctggtgcacagcaacg | |
| | | gcaacacccacctgcactggtaccagcagagacccggacaggctcccaggctgctg | |
| | | atctacagcgtgagcaacaggttctccgaggtgcctgccaggtttagcggcagcgga | |
| | | agcggcaccgactttaccctgaccatcagcagcgtggagtccgaggacttcgccgtg | |
| | | tattactgcagccagaccagccacatcccttacaccttcggcggcggcaccaagctg | |
| | | gagatcaaa | |
| CD8TM | CD8 | agtgctgctgcctttgtcccggtatttctcccagccaaaccgaccacgactcccgcccc | 28 |
| | transmembrane | gcgccctccgacacccgctcccaccatcgcctctcaacctcttagtcttcgccccgag | |
| | | gcatgccgacccgccgccgggggtgctgttcatacgaggggcttggacttcgcttgt | |
| | | gatatttacatttgggctccgttggcgggtacgtgcggcgtccttttgttgtcactcgttat | |
| | | tactttgtattgtaatcacaggaatcgc | |
| 41BB co- | 41BB co- | aaacggggcagaaagaaactcctgtatatattcaaacaaccatttatgagaccagtac | 29 |
| stim | stimulatory | aaactactcaagaggaagatggctgtagctgccgatttccagaagaagaagaagga | |
| domain | domain | ggatgtgaactg | |
| CD3Z | CD3Z domain | cgagtgaagttttcccgaagcgcagacgctccggcatatcagcaaggacagaatcag | 30 |
| domain | | ctgtataacgaactgaatttgggacgccgcgaggagtatgacgtgcttgataaacgcc | |
| | | gggggagagacccggaaatggggggtaaaccccgaagaaagaatccccaagaag | |
| | | gactctacaatgaactccagaaggataagatggcggaggcctactcagaaataggta | |
| | | tgaagggcgaacgacgacggggaaaaggtcacgatggcctctaccaagggttgagt | |
| | | acggcaaccaaagatacgtacgatgcactgcatatgcaggccctgcctcccaga | |
| bGH | bGH poly(A) | ctgtgccttctagttgccagccatctgttgtttgcccctcccccgtgccttccttgaccctg | 31 |
| poly(A) | signal | gaaggtgccactcccactgtcctttcctaataaaatgaggaaattgcatcgcattgtctg | |
| signal | | agtaggtgtcattctattctggggggtggggtggggcaggacagcaagggggagga | |
| | | ttgggaagacaatagcaggcatgctggggatgcggtgggctctatgg | |
| RHA- | Right homology | tgaccagatggacctggctggagaagaagagattgagctctactcaggtgggccctc | 32 |
| CIITA | arm CIITA | ctccctctggtctcttccggtatcccccacccctcagcttgctgtagagacggcaatca | |
| | | ggggaaattctggtccctgccctcccgtcagcaccacggacagctcccacgtctgtg | |
| | | ggacgctctctgcagatggggatgatctcccagccctgccccgcctctccctcgttcc | |
| | | ccaccagccctctttccagaaatttccttcttcatccaagggacttttcctcccagaaccc | |
| | | gacacagacaccatcaactgcgaccagttcagcaggctgttgtgtgacatggaaggt | |
| | | gatgaagagaccagggaggcttatgccaatatcggtgaggaagcacctgagcccag | |
| | | aaaaggacaatcaagggcaagagttctttgctgccacttgtcaatatcacccattcatca | |
| | | tgagccacgtcagtcccctcccacagaaatcattgcaagggggatgcggagcaatg | |
| | | gctggaggaacggagactccagggaagagaggggagatggaggccagtggggg | |
| | | aaataggccccttcactaatgaccaccaagaaaacaaaatctcatgtttacatcctcca | |
| | | cctccatttctatacgcatttctgcttcttgctcttctgtccatcctttctacaaagcccatac | |
| | | catacacccctttcccttttcctcccagctccttagccaagctactctagtatttgtaataa | |
| | | ctagcatttactggatactcatagtatgctcattgc | |
| Right ITR | Right inverted | aggaacccctagtgatggagttggccactccctctctgcgcgctcgctcgctcactga | 33 |
| | terminal repeat | ggccgggcgaccaaaggtcgcccgacgcccgggctttgcccgggcggcctcagtg | |
| | | agcgagcgagcgcgcagctgcctgcagg | |
| B2M-2 | gRNA target | GGCCGAGATGTCTCGCTCCG | 34, |
| gRNA | sequence in | | 180 |
| (Exon | B2M, spacer | | |
| 1_T2) | sequence | | |
| FAS Ex1 | gRNA target | GGATTGCTCAACAACCATGC | 35, |
| T7 | sequence in FAS, | | 181 |
| | spacer sequence | | |
| LHA-B2M | Left homology | gttctagggtggaaactaagagaatgatgtacctagagggcgctggaagctctaaag | 36 |
| | arm B2M | ccctagcagttactgcttttactattagtggtcgtttttttctcccccccgccccccgacaa | |
| | | atcaacagaacaaagaaaattacctaaacagcaaggacatagggaggaacttcttgg | |
| | | cacagaactttccaaacactttttcctgaagggatacaagaagcaagaaaggtactcttt | |
| | | cactaggaccttctctgagctgtcctcaggatgcttttgggactatttttcttacccagag | |
| | | aatggagaaaccctgcagggaattcccaagctgtagttataaacagaagttctccttct | |
| | | gctaggtagcattcaaagatcttaatcttctgggtttccgttttctcgaatgaaaaatgca | |
| | | ggtccgagcagttaactggctggggcaccattagcaagtcacttagcatctctggggc | |
| | | cagtctgcaaagcgagggggcagccttaatgtgcctccagcctgaagtcctagaatg | |
| | | agcgcccggtgtcccaagctggggcgcgcaccccagatcggagggcgccgatgta | |
| | | cagacagcaaactcacccagtctagtgcatgccttcttaaacatcacgagactctaaga | |
| | | aaaggaaactgaaaacgggaaagtccctctctctaacctggcactgcgtcgctggctt | |
| | | ggagacaggtgacggtccctgcgggccttgtcctgattggctgggcacgcgtttaata | |
| | | taagtggaggcgtcgcgctggcgggcattcctgaagct | |
| FAS Ex1 | gRNA target in | GATTGCTCAACAACCATGCT | 37, |
| T9 | FAS, spacer | | 182 |
| | sequence | | |
| FAS Ex2 | gRNA target in | GTGACTGACATCAACTCCAA | 38, |
| T1 | FAS, spacer | | 183 |
| | sequence | | |
| FAS Ex2 | gRNA target in | CACTTGGGCATTAACACTTT | 39, |
| T2 | FAS, spacer | | 184 |
| | sequence | | |
| IgE signal | IgE signal | atggactggacctggatcctgttcctggtggccgccgccaccagggtgcacagc | 40 |
| peptide | peptide-B2M | | |
| | plasmid | | |
| IL15 CDS | IL15 Coding | ggcattcatgtcttcattttgggctgtttcagtgcagggcttcctaaaacagaagccaact | 41 |
| | sequence | gggtgaatgtaataagtgatttgaaaaaaattgaagatcttattcaatctatgcatattgat | |
| | | gctactttatatacggaaagtgatgttcaccccagttgcaaagtaacagcaatgaagtg | |
| | | ctttctcttggagttacaagttatttcacttgagtccggagatgcaagtattcatgatacag | |
| | | tagaaaatctgatcatcctagcaaacaacagtttgtcttctaatgggaatgtaacagaat | |
| | | ctggatgcaaagaatgtgaggaactggaggaaaaaaatattaaagaatttttgcagag | |
| | | ttttgtacatattgtccaaatgttcatcaacacttct | |
| linker | Linker-B2M | agcggcggcggcagcggcggcggcggcagcggcggcggcggcagcggcggc | 42 |
| | plasmid | ggcggcagcggcggcggcagcctgcag | |
| IR15Rα | IL15Rα coding | atcacgtgccctccccccatgtccgtggaacacgcagacatctgggtcaagagctac | 43 |
| CDS | sequence | agcttgtactccagggagcggtacatttgtaactctggtttcaagcgtaaagccggcac | |
| | | gtccagcctgacggagtgcgtgttgaacaaggccacgaatgtcgcccactggacaa | |
| | | cccccagtctcaaatgcattagagaccctgccctggttcaccaaaggccagcgccac | |
| | | cctccacagtaacgacggcaggggtgaccccacagccagagagcctctccccttct | |
| | | ggaaaagagcccgcagcttcatctcccagctcaaacaacacagcggccacaacagc | |
| | | agctattgtcccgggctcccagctgatgccttcaaaatcaccttccacaggaaccaca | |
| | | gagataagcagtcatgagtcctcccacggcaccccctctcagacaacagccaagaa | |
| | | ctgggaactcacagcatccgcctcccaccagccgccaggtgtgtatccacagggcc | |
| | | acagcgacaccactgtggctatctccacgtccactgtcctgctgtgtgggctgagcgc | |
| | | tgtgtctctcctggcatgctacctcaagtcaaggcaaactcccccgctggccagcgttg | |
| | | aaatggaagccatggaggctctgccggtgacttgggggaccagcagcagagatgaa | |
| | | gacttggaaaactgctctcaccaccta | |
| GSG tag | GSG tag | ggaagcgga | 44 |
| P2A | P2A | gctactaacttcagcctgctgaagcaggctggagacgtggaggagaaccctggacct | 45 |
| B2M signal | B2M signal | atgtctcgctccgttgccttagctgtgctcgcgctactctctctttctggattagaggct | 46 |
| sequence | sequence | | |
| HLA-G | HLA-G peptide | gtcatggcgccccgaaccctcttcctg | 47 |
| peptide | | | |
| GS linker | GS linker | ggtggaggcggttcaggcggaggtggctctggcggtggcggatcg | 48 |
| B2M | B2M | atccagcgtactccaaagattcaggtttactcacgtcatccagcagagaatggaaagtc | 49 |
| | | aaatttcctgaattgctatgtgtctgggtttcatccatccgacattgaagttgacttactga | |
| | | agaatggagagagaattgaaaaagtggagcattcagacttgtctttcagcaaggactg | |
| | | gtctttctatctcttgtactacactgaattcacccccactgaaaaagatgagtatgcctgc | |
| | | cgtgtgaaccatgtgactttgtcacagcccaagatagttaagtgggatcgagacatg | |
| GS linker | GS linker | ggtggtggtggttctggtggtggtggttctggcggcggcggctccggtggtggtgga | 50 |
| | | tcc | |
| HLA-E | HLA-E coding | ggctcccactccttgaagtatttccacacttccgtgtcccggcccggccgcggggagc | 51 |
| | sequence | cccgcttcatctctgtgggctacgtggacgacacccagttcgtgcgcttcgacaacga | |
| | | cgccgcgagtccgaggatggtgccgcgggcgccgtggatggagcaggaggggtc | |
| | | agagtattgggaccgggagacacggagcgccagggacaccgcacagattttccga | |
| | | gtgaatctgcggacgctgcgcggctactacaatcagagcgaggccgggtctcacac | |
| | | cctgcagtggatgcatggctgcgagctggggcccgacgggcgcttcctccgcgggt | |
| | | atgaacagttcgcctacgacggcaaggattatctcaccctgaatgaggacctgcgctc | |
| | | ctggaccgcggtggacacggcggctcagatctccgagcaaaagtcaaatgatgcct | |
| | | ctgaggcggagcaccagagagcctacctggaagacacatgcgtggagtggctcca | |
| | | caaatacctggagaaggggaaggagacgctgcttcacctggagcccccaaagaca | |
| | | cacgtgactcaccaccccatctctgaccatgaggccaccctgaggtgctgggccctg | |
| | | ggcttctaccctgcggagatcacactgacctggcagcaggatggggagggccatac | |
| | | ccaggacacggagctcgtggagaccaggcctgcaggggatggaaccttccagaag | |
| | | tgggcagctgtggtggtgccttctggagaggagcagagatacacgtgccatgtgcag | |
| | | catgaggggctacccgagcccgtcaccctgagatggaagccggcttcccagcccac | |
| | | catccccatcgtgggcatcattgctggcctggttctccttggatctgtggtctctggagct | |
| | | gtggttgctgctgtgatatggaggaagaagagctcaggtggaaaaggagggagcta | |
| | | ctctaaggctgagtggagcgacagtgcccaggggtctgagtctcacagcttg | |
| 3X Stop | 3X Stop codons | TAATGATAG | 52 |
| codons | | | |
| FAS Ex2 | gRNA target in | TTGGAAGGCCTGCATCATGA | 53, |
| T3 | FAS, spacer | | 185 |
| | sequence | | |
| RHA-B2M | Right homology | ccagcgtgagtctctcctaccctcccgctctggtccttcctctcccgctctgcaccctct | 54 |
| | Arm-B2M | gtggccctcgctgtgctctctcgctccgtgacttcccttctccaagttctccttggtggcc | |
| | | cgccgtggggctagtccagggctggatctcggggaagcggcggggtggcctggga | |
| | | gtggggaagggggtgcgcacccgggacgcgcgctacttgcccctttcggcgggga | |
| | | gcaggggagacctttggcctacggcgacgggagggtcgggacaaagtttagggcg | |
| | | tcgataagcgtcagagcgccgaggttgggggagggtttctcttccgctctttcgcggg | |
| | | gcctctggctcccccagcgcagctggagtgggggacgggtaggctcgtcccaaag | |
| | | gcgcggcgctgaggtttgtgaacgcgtggaggggcgcttggggtctgggggaggc | |
| | | gtcgcccgggtaagcctgtctgctgcggctctgcttcccttagactggagagctgtgg | |
| | | acttcgtctaggcgcccgctaagttcgcatgtcctagcacctctgggtctatgtggggc | |
| | | cacaccgtggggaggaaacagcacgcgacgtttgtagaatgcttggctgtgatacaa | |
| | | agcggtttcgaataattaacttatttgttcccatcacatgtcacttttaaaaaattataagaa | |
| | | ctacccgttattgacatctttctgtgtgccaaggactttatgtgctttgcgtcatttaattttg | |
| | | aaaacagttatcttccgccatagataactactatggttatcttct | |
| FAS Ex2 | gRNA target in | ACTCCAAGGGATTGGAATTG | 55, |
| T7 | FAS, spacer | | 186 |
| | sequence | | |
| B2MF2 | B2M Indel | CAGACAGCAAACTCACCCAG | 56 |
| | Primer | | |
| B2MR2 | B2M Indel | AAACTTTGTCCCGACCCTCC | 57 |
| | Primer | | |
| B2M-geno- | B2M Zygosity | AAAAGATCTGTGGACTCCAC | 58 |
| F1 | primer | CACCACGAAATGGCGGCACC | |
| | | TTATTTATGGTC | |
| B2M-geno- | B2M Zygosity | GCTCTGGAGAATCTCACGCA | 59 |
| R1 | primer | GAAGGCAGGCGTTTTTCTTA | |
| | | AAAAAAAATGCACGAATTA | |
| Poly-A-F | B2M knock-in | AGGATTGGGAAGACAATAGC | 60 |
| | primer | AGGCATGCTGGGGATGCGGTGG | |
| B2M-geno- | B2M knock-in | GCTCTGGAGAATCTCACGCAGA | 61 |
| R1 | primer | AGGCAGGCGTTTTTCTTAAAAA | |
| | | AAAATGCACGAATTA | |
| CIITA- | CIITA Zygosity | GCCCCACCCCTCCTACTTTATGT | 62 |
| OUT-F | primer | CTCCATGGATTTGCCTGTTTTGG | |
| | | TCATTTCA | |
| CIITA- | CIITA Zygosity | CTCTAATGCAAACTTGGGTAGGT | 63 |
| OUT-R | primer | CGTTTCACCTCTCTAAACCTCAAT | |
| | | TTCCTCATTTG | |
| CD3Z-seq- | CIITA Knock-in | GAGTGAAGTTTTCCCGAAGCGCA | 64 |
| F1 | Primer | GACGCTCCGGCATATCAGCAAGG | |
| | | ACAG | |
| CIITA- | CIITA Knock-in | CTCTAATGCAAACTTGGGTAGGTC | 65 |
| OUT-R | Primer | GTTTCACCTCTCTAAACCTCAATTT | |
| | | CCTCATTTG | |
| BCMA- | CAG version | CCTGCAGGCAGCTGCGCGCTCGCTCGCTCACTGAGG | 66 |
| CAR | CIITA HA- | CCGCCCGGGCGTCGGGCGACCTTTGGTCGCCCGGCC | |
| plasmid | BCMA-CAR | TCAGTGAGCGAGCGAGCGCGCAGAGAGGGAGTGGC | |
| | sequence | CAACTCCATCACTAGGGGTTCCTGCGGCCGCACGCG | |
| | | TCATATTTATGGGGTATATGTGAATATTTATTACAT | |
| | | GCATAGAAGGTATAATGATCATGTCAGGATATTTGA | |
| | | GGTATCCACATTTGGGATTGTTTAAAGATTAAATGA | |
| | | AATAGTGTTAAAAGTATTTAATATGCCCTTCAACAA | |
| | | ATGATGAGGAAATCTTAGAATCTGCTCAGACTCCTT | |
| | | CAGTTTACATATTAGGAAACTGAGGCACAGAAAGG | |
| | | AGCAGAGACTTGCTCAAGTCCACCCAAAGCAGTAG | |
| | | AGCATTGTGGTTAAATGCAGGACTTCAGTCAGACTG | |
| | | TCTGGGTTCAAATCCTGGTTCCACTTGGACATGGGT | |
| | | TTCCTTACATAAATCACTTCACCTCTCTGAGCCTCAG | |
| | | TTTTCTCATATGCAAAGTGAGGATAATAATAATACC | |
| | | TTCCTTACATGGTTACTGATATGAGTATTAAATGTG | |
| | | CCAGCTCATGTGCCTGGCGTATAGGAGGTGCTTTAT | |
| | | AAACCTTAGCTGTTACCACTCATGGCATTGCCAAAT | |
| | | GTGGGACGGGTCTCCTGACTCTCTGGTGTGAGATTG | |
| | | ATGGAATCCACACTTTCCAGTTCCCTTTTCTACCTCC | |
| | | TGGGTATCTTCTCATATGGTTGTAAGTTCCTTGGAG | |
| | | GAAGGGAATGTGGCTTGCTCTCTCCACCACGCTGAG | |
| | | CATATAAGAGGTGCTGAATGAGCGCTTTTATTCACT | |
| | | CCTCTCATCCCCAGCCCTCACCAGCTGGGAGTTGTT | |
| | | GTAGGTGTCAATTTTCTGCCTCTTTCCAACACCCTGT | |
| | | GAGGTGACTGAGCATTGTCTTCCCTCCCAGGCAGCT | |
| | | CACAGTGTAAGCTTGTGGACGATATCGAATTCGCAC | |
| | | GACATTGATTATTGACTAGTTATTAATAGTAATCAA | |
| | | TTACGGGGTCATTAGTTCATAGCCCATATATGGAGT | |
| | | TCCGCGTTACATAACTTACGGTAAATGGCCCGCCTG | |
| | | GCTGACCGCCCAACGACCCCCGCCCATTGACGTCAA | |
| | | TAATGACGTATGTTCCCATAGTAACGCCAATAGGGA | |
| | | CTTTCCATTGACGTCAATGGGTGGACTATTTACGGT | |
| | | AAACTGCCCACTTGGCAGTACATCAAGTGTATCATA | |
| | | TGCCAAGTACGCCCCCTATTGACGTCAATGACGGTA | |
| | | AATGGCCCGCCTGGCATTATGCCCAGTACATGACCT | |
| | | TATGGGACTTTCCTACTTGGCAGTACATCTACGTAT | |
| | | TAGTCATCGCTATTACCATGGGTCGAGGTGAGCCCC | |
| | | ACGTTCTGCTTCACTCTCCCCATCTCCCCCCCCTCCC | |
| | | CACCCCCAATTTTGTATTTATTTATTTTTTAATTATTT | |
| | | TGTGCAGCGATGGGGGCGGGGGGGGGGGGGGCGCG | |
| | | CGCCAGGCGGGGCGGGGCGGGGCGAGGGGCGGGG | |
| | | CGGGGCGAGGCGGAGAGGTGCGGCGGCAGCCAATC | |
| | | AGAGCGGCGCGCTCCGAAAGTTTCCTTTTATGGCGA | |
| | | GGCGGCGGCGGCGGCGGCCCTATAAAAAGCGAAGC | |
| | | GCGCGGCGGGCGGGAGTCGCTGCGTTGCCTTCGCCC | |
| | | CGTGCCCCGCTCCGCGCCGCCTCGCGCCGCCCGCCC | |
| | | CGGCTCTGACTGACCGCGTTACTCCCACAGGTGAGC | |
| | | GGGCGGGACGGCCCTTCTCCTCCGGGCTGTAATTAG | |
| | | CGCTTGGTTTAATGACGGCTCGTTTCTTTTCTGTGGC | |
| | | TGCGTGAAAGCCTTAAAGGGCTCCGGGAGGGCCCT | |
| | | TTGTGCGGGGGGGAGCGGCTCGGGGGGTGCGTGCG | |
| | | TGTGTGTGTGCGTGGGGAGCGCCGCGTGCGGCCCGC | |
| | | GCTGCCCGGCGGCTGTGAGCGCTGCGGGCGCGGCG | |
| | | CGGGGCTTTGTGCGCTCCGCGTGTGCGCGAGGGGA | |
| | | GCGCGGCCGGGGGCGGTGCCCCGCGGTGCGGGGGG | |
| | | GCTGCGAGGGGAACAAAGGCTGCGTGCGGGGTGTG | |
| | | TGCGTGGGGGGGTGAGCAGGGGGTGTGGGCGCGGC | |
| | | GGTCGGGCTGTAACCCCCCCCTGCACCCCCCTCCCC | |
| | | GAGTTGCTGAGCACGGCCCGGCTTCGGGTGCGGGG | |
| | | CTCCGTGCGGGGCGTGGCGCGGGGCTCGCCGTGCC | |
| | | GGGCGGGGGGTGGCGGCAGGTGGGGGTGCCGGGCG | |
| | | GGGCGGGGCCGCCTCGGGCCGGGGAGGGCTCGGGG | |
| | | GAGGGGCGCGGCGGCCCCGGAGCGCCGGCGGCTGT | |
| | | CGAGGCGCGGCGAGCCGCAGCCATTGCCTTTTATGG | |
| | | TAATCGTGCGAGAGGGCGCAGGGACTTCCTTTGTCC | |
| | | CAAATCTGGCGGAGCCGAAATCTGGGAGGCGCCGC | |
| | | CGCACCCCCTCTAGCGGGCGCGGGCGAAGCGGTGC | |
| | | GGCGCCGGCAGGAAGGAAATGGGCGGGGAGGGCCT | |
| | | TCGTGCGTCGCCGCGCCGCCGTCCCCTTCTCCATCTC | |
| | | CAGCCTCGGGGCTGCCGCAGGGGGACGGCTGCCTT | |
| | | CGGGGGGGACGGGGCAGGGCGGGGTTCGGCTTCTG | |
| | | GCGTGTGACCGGCGGCTCTAGAGCCTCTGCTAACCA | |
| | | TGTTCATGCCTTCTTCTTTTTCCTACAGGGGGGATCC | |
| | | GTTTATCTGCAGAATTCGCCCTTGACGTCGCCACCA | |
| | | TGGCGCTTCCGGTGACAGCACTGCTCCTCCCCTTGG | |
| | | CGCTGTTGCTCCACGCAGCAAGGCCGCAGGTGCAG | |
| | | CTGGTGCAGAGCGGAGCCGAGCTCAAGAAGCCCGG | |
| | | AGCCTCCGTGAAGGTGAGCTGCAAGGCCAGCGGCA | |
| | | ACACCCTGACCAACTACGTGATCCACTGGGTGAGAC | |
| | | AAGCCCCCGGCCAAAGGCTGGAGTGGATGGGCTAC | |
| | | ATCCTGCCCTACAACGACCTGACCAAGTACAGCCAG | |
| | | AAGTTCCAGGGCAGGGTGACCATCACCAGGGATAA | |
| | | GAGCGCCTCCACCGCCTATATGGAGCTGAGCAGCCT | |
| | | GAGGAGCGAGGACACCGCTGTGTACTACTGTACAA | |
| | | GGTGGGACTGGGACGGCTTCTTTGACCCCTGGGGCC | |
| | | AGGGCACAACAGTGACCGTCAGCAGCGGCGGCGGA | |
| | | GGCAGCGGCGGCGGCGGCAGCGGCGGAGGCGGAA | |
| | | GCGAAATCGTGATGACCCAGAGCCCCGCCACACTG | |
| | | AGCGTGAGCCCTGGCGAGAGGGCCAGCATCTCCTG | |
| | | CAGGGCTAGCCAAAGCCTGGTGCACAGCAACGGCA | |
| | | ACACCCACCTGCACTGGTACCAGCAGAGACCCGGA | |
| | | CAGGCTCCCAGGCTGCTGATCTACAGCGTGAGCAAC | |
| | | AGGTTCTCCGAGGTGCCTGCCAGGTTTAGCGGCAGC | |
| | | GGAAGCGGCACCGACTTTACCCTGACCATCAGCAG | |
| | | CGTGGAGTCCGAGGACTTCGCCGTGTATTACTGCAG | |
| | | CCAGACCAGCCACATCCCTTACACCTTCGGCGGCGG | |
| | | CACCAAGCTGGAGATCAAAAGTGCTGCTGCCTTTGT | |
| | | CCCGGTATTTCTCCCAGCCAAACCGACCACGACTCC | |
| | | CGCCCCGCGCCCTCCGACACCCGCTCCCACCATCGC | |
| | | CTCTCAACCTCTTAGTCTTCGCCCCGAGGCATGCCG | |
| | | ACCCGCCGCCGGGGGTGCTGTTCATACGAGGGGCTT | |
| | | GGACTTCGCTTGTGATATTTACATTTGGGCTCCGTTG | |
| | | GCGGGTACGTGCGGCGTCCTTTTGTTGTCACTCGTT | |
| | | ATTACTTTGTATTGTAATCACAGGAATCGCAAACGG | |
| | | GGCAGAAAGAAACTCCTGTATATATTCAAACAACC | |
| | | ATTTATGAGACCAGTACAAACTACTCAAGAGGAAG | |
| | | ATGGCTGTAGCTGCCGATTTCCAGAAGAAGAAGAA | |
| | | GGAGGATGTGAACTGCGAGTGAAGTTTTCCCGAAG | |
| | | CGCAGACGCTCCGGCATATCAGCAAGGACAGAATC | |
| | | AGCTGTATAACGAACTGAATTTGGGACGCCGCGAG | |
| | | GAGTATGACGTGCTTGATAAACGCCGGGGGAGAGA | |
| | | CCCGGAAATGGGGGGTAAACCCCGAAGAAAGAATC | |
| | | CCCAAGAAGGACTCTACAATGAACTCCAGAAGGAT | |
| | | AAGATGGCGGAGGCCTACTCAGAAATAGGTATGAA | |
| | | GGGCGAACGACGACGGGGAAAAGGTCACGATGGCC | |
| | | TCTACCAAGGGTTGAGTACGGCAACCAAAGATACG | |
| | | TACGATGCACTGCATATGCAGGCCCTGCCTCCCAGA | |
| | | TAATCCGCTGATCAGCCTCGACTGTGCCTTCTAGTT | |
| | | GCCAGCCATCTGTTGTTTGCCCCTCCCCCGTGCCTTC | |
| | | CTTGACCCTGGAAGGTGCCACTCCCACTGTCCTTTC | |
| | | CTAATAAAATGAGGAAATTGCATCGCATTGTCTGAG | |
| | | TAGGTGTCATTCTATTCTGGGGGGTGGGGTGGGGCA | |
| | | GGACAGCAAGGGGGAGGATTGGGAAGACAATAGC | |
| | | AGGCATGCTGGGGATGCGGTGGGCTCTATGGGTCG | |
| | | ACTGACCAGATGGACCTGGCTGGAGAAGAAGAGAT | |
| | | TGAGCTCTACTCAGGTGGGCCCTCCTCCCTCTGGTC | |
| | | TCTTCCGGTATCCCCCACCCCTCAGCTTGCTGTAGA | |
| | | GACGGCAATCAGGGGAAATTCTGGTCCCTGCCCTCC | |
| | | CGTCAGCACCACGGACAGCTCCCACGTCTGTGGGAC | |
| | | GCTCTCTGCAGATGGGGATGATCTCCCAGCCCTGCC | |
| | | CCGCCTCTCCCTCGTTCCCCACCAGCCCTCTTTCCAG | |
| | | AAATTTCCTTCTTCATCCAAGGGACTTTTCCTCCCAG | |
| | | AACCCGACACAGACACCATCAACTGCGACCAGTTC | |
| | | AGCAGGCTGTTGTGTGACATGGAAGGTGATGAAGA | |
| | | GACCAGGGAGGCTTATGCCAATATCGGTGAGGAAG | |
| | | CACCTGAGCCCAGAAAAGGACAATCAAGGGCAAGA | |
| | | GTTCTTTGCTGCCACTTGTCAATATCACCCATTCATC | |
| | | ATGAGCCACGTCAGTCCCCTCCCACAGAAATCATTG | |
| | | CAAGGGGGATGCGGAGCAATGGCTGGAGGAACGGA | |
| | | GACTCCAGGGAAGAGAGGGGAGATGGAGGCCAGTG | |
| | | GGGGAAATAGGCCCCTTCACTAATGACCACCAAGA | |
| | | AAACAAAATCTCATGTTTACATCCTCCACCTCCATT | |
| | | TCTATACGCATTTCTGCTTCTTGCTCTTCTGTCCATC | |
| | | CTTTCTACAAAGCCCATACCATACACCCCTTTCCCTT | |
| | | TTCCTCCCAGCTCCTTAGCCAAGCTACTCTAGTATTT | |
| | | GTAATAACTAGCATTTACTGGATACTCATAGTATGC | |
| | | TCATTGCTGTCCGGTAACCACGTGCGGACCGAGGCT | |
| | | GCAGCGTCGTCCTCCCTAGGAACCCCTAGTGATGGA | |
| | | GTTGGCCACTCCCTCTCTGCGCGCTCGCTCGCTCACT | |
| | | GAGGCCGGGCGACCAAAGGTCGCCCGACGCCCGGG | |
| | | CTTTGCCCGGGCGGCCTCAGTGAGCGAGCGAGCGC | |
| | | GCAGCTGCCTGCAGGGGCGCCTGATGCGGTATTTTC | |
| | | TCCTTACGCATCTGTGCGGTATTTCACACCGCATAC | |
| | | GTCAAAGCAACCATAGTACGCGCCCTGTAGCGGCG | |
| | | CATTAAGCGCGGCGGGTGTGGTGGTTACGCGCAGC | |
| | | GTGACCGCTACACTTGCCAGCGCCCTAGCGCCCGCT | |
| | | CCTTTCGCTTTCTTCCCTTCCTTTCTCGCCACGTTCG | |
| | | CCGGCTTTCCCCGTCAAGCTCTAAATCGGGGGCTCC | |
| | | CTTTAGGGTTCCGATTTAGTGCTTTACGGCACCTCG | |
| | | ACCCCAAAAAACTTGATTTGGGTGATGGTTCACGTA | |
| | | GTGGGCCATCGCCCTGATAGACGGTTTTTCGCCCTT | |
| | | TGACGTTGGAGTCCACGTTCTTTAATAGTGGACTCT | |
| | | TGTTCCAAACTGGAACAACACTCAACCCTATCTCGG | |
| | | GCTATTCTTTTGATTTATAAGGGATTTTGCCGATTTC | |
| | | GGCCTATTGGTTAAAAAATGAGCTGATTTAACAAAA | |
| | | ATTTAACGCGAATTTTAACAAAATATTAACGTTTAC | |
| | | AATTTTATGGTGCACTCTCAGTACAATCTGCTCTGA | |
| | | TGCCGCATAGTTAAGCCAGCCCCGACACCCGCCAAC | |
| | | ACCCGCTGACGCGCCCTGACGGGCTTGTCTGCTCCC | |
| | | GGCATCCGCTTACAGACAAGCTGTGACCGTCTCCGG | |
| | | GAGCTGCATGTGTCAGAGGTTTTCACCGTCATCACC | |
| | | GAAACGCGCGAGACGAAAGGGCCTCGTGATACGCC | |
| | | TATTTTTATAGGTTAATGTCATGAACAATAAAACTG | |
| | | TCTGCTTACATAAACAGTAATACAAGGGGTGTTATG | |
| | | AGCCATATTCAACGGGAAACGTCGAGGCCGCGATT | |
| | | AAATTCCAACATGGATGCTGATTTATATGGGTATAA | |
| | | ATGGGCTCGCGATAATGTCGGGCAATCAGGTGCGA | |
| | | CAATCTATCGCTTGTATGGGAAGCCCGATGCGCCAG | |
| | | AGTTGTTTCTGAAACATGGCAAAGGTAGCGTTGCCA | |
| | | ATGATGTTACAGATGAGATGGTCAGACTAAACTGG | |
| | | CTGACGGAATTTATGCCTCTTCCGACCATCAAGCAT | |
| | | TTTATCCGTACTCCTGATGATGCATGGTTACTCACC | |
| | | ACTGCGATCCCCGGAAAAACAGCATTCCAGGTATTA | |
| | | GAAGAATATCCTGATTCAGGTGAAAATATTGTTGAT | |
| | | GCGCTGGCAGTGTTCCTGCGCCGGTTGCATTCGATT | |
| | | CCTGTTTGTAATTGTCCTTTTAACAGCGATCGCGTAT | |
| | | TTCGTCTCGCTCAGGCGCAATCACGAATGAATAACG | |
| | | GTTTGGTTGATGCGAGTGATTTTGATGACGAGCGTA | |
| | | ATGGCTGGCCTGTTGAACAAGTCTGGAAAGAAATG | |
| | | CATAAACTTTTGCCATTCTCACCGGATTCAGTCGTC | |
| | | ACTCATGGTGATTTCTCACTTGATAACCTTATTTTTG | |
| | | ACGAGGGGAAATTAATAGGTTGTATTGATGTTGGAC | |
| | | GAGTCGGAATCGCAGACCGATACCAGGATCTTGCC | |
| | | ATCCTATGGAACTGCCTCGGTGAGTTTTCTCCTTCAT | |
| | | TACAGAAACGGCTTTTTCAAAAATATGGTATTGATA | |
| | | ATCCTGATATGAATAAATTGCAGTTTCATTTGATGC | |
| | | TCGATGAGTTTTTCTAATCTCATGACCAAAATCCCTT | |
| | | AACGTGAGTTTTCGTTCCACTGAGCGTCAGACCCCG | |
| | | TAGAAAAGATCAAAGGATCTTCTTGAGATCCTTTTT | |
| | | TTCTGCGCGTAATCTGCTGCTTGCAAACAAAAAAAC | |
| | | CACCGCTACCAGCGGTGGTTTGTTTGCCGGATCAAG | |
| | | AGCTACCAACTCTTTTTCCGAAGGTAACTGGCTTCA | |
| | | GCAGAGCGCAGATACCAAATACTGTCCTTCTAGTGT | |
| | | AGCCGTAGTTAGGCCACCACTTCAAGAACTCTGTAG | |
| | | CACCGCCTACATACCTCGCTCTGCTAATCCTGTTAC | |
| | | CAGTGGCTGCTGCCAGTGGCGATAAGTCGTGTCTTA | |
| | | CCGGGTTGGACTCAAGACGATAGTTACCGGATAAG | |
| | | GCGCAGCGGTCGGGCTGAACGGGGGGTTCGTGCAC | |
| | | ACAGCCCAGCTTGGAGCGAACGACCTACACCGAAC | |
| | | TGAGATACCTACAGCGTGAGCTATGAGAAAGCGCC | |
| | | ACGCTTCCCGAAGGGAGAAAGGCGGACAGGTATCC | |
| | | GGTAAGCGGCAGGGTCGGAACAGGAGAGCGCACGA | |
| | | GGGAGCTTCCAGGGGGAAACGCCTGGTATCTTTATA | |
| | | GTCCTGTCGGGTTTCGCCACCTCTGACTTGAGCGTC | |
| | | GATTTTTGTGATGCTCGTCAGGGGGGCGGAGCCTAT | |
| | | GGAAAAACGCCAGCAACGCGGCCTTTTTACGGTTCC | |
| | | TGGCCTTTTGCTGGCCTTTTGCTCACATGT | |
| B2M | B2M-CAGGS- | CCTGCAGGCAGCTGCGCGCTCGCTCGCTCACTGAGG | 67 |
| plasmid | IL15-IR15 | CCGCCCGGGCGTCGGGCGACCTTTGGTCGCCCGGCC | |
| | fusion-P2A- | TCAGTGAGCGAGCGAGCGCGCAGAGAGGGAGTGGC | |
| | HLA-E sequence | CAACTCCATCACTAGGGGTTCCTGCGGCCGCACGCG | |
| | | TGTTCTAGGGTGGAAACTAAGAGAATGATGTACCTA | |
| | | GAGGGCGCTGGAAGCTCTAAAGCCCTAGCAGTTAC | |
| | | TGCTTTTACTATTAGTGGTCGTTTTTTTCTCCCCCCC | |
| | | GCCCCCCGACAAATCAACAGAACAAAGAAAATTAC | |
| | | CTAAACAGCAAGGACATAGGGAGGAACTTCTTGGC | |
| | | ACAGAACTTTCCAAACACTTTTTCCTGAAGGGATAC | |
| | | AAGAAGCAAGAAAGGTACTCTTTCACTAGGACCTTC | |
| | | TCTGAGCTGTCCTCAGGATGCTTTTGGGACTATTTTT | |
| | | CTTACCCAGAGAATGGAGAAACCCTGCAGGGAATT | |
| | | CCCAAGCTGTAGTTATAAACAGAAGTTCTCCTTCTG | |
| | | CTAGGTAGCATTCAAAGATCTTAATCTTCTGGGTTT | |
| | | CCGTTTTCTCGAATGAAAAATGCAGGTCCGAGCAGT | |
| | | TAACTGGCTGGGGCACCATTAGCAAGTCACTTAGCA | |
| | | TCTCTGGGGCCAGTCTGCAAAGCGAGGGGGCAGCC | |
| | | TTAATGTGCCTCCAGCCTGAAGTCCTAGAATGAGCG | |
| | | CCCGGTGTCCCAAGCTGGGGCGCGCACCCCAGATC | |
| | | GGAGGGCGCCGATGTACAGACAGCAAACTCACCCA | |
| | | GTCTAGTGCATGCCTTCTTAAACATCACGAGACTCT | |
| | | AAGAAAAGGAAACTGAAAACGGGAAAGTCCCTCTC | |
| | | TCTAACCTGGCACTGCGTCGCTGGCTTGGAGACAGG | |
| | | TGACGGTCCCTGCGGGCCTTGTCCTGATTGGCTGGG | |
| | | CACGCGTTTAATATAAGTGGAGGCGTCGCGCTGGCG | |
| | | GGCATTCCTGAAGCTAAGCTTGTGGACGATATCGAA | |
| | | TTCGCACGACATTGATTATTGACTAGTTATTAATAG | |
| | | TAATCAATTACGGGGTCATTAGTTCATAGCCCATAT | |
| | | ATGGAGTTCCGCGTTACATAACTTACGGTAAATGGC | |
| | | CCGCCTGGCTGACCGCCCAACGACCCCCGCCCATTG | |
| | | ACGTCAATAATGACGTATGTTCCCATAGTAACGCCA | |
| | | ATAGGGACTTTCCATTGACGTCAATGGGTGGACTAT | |
| | | TTACGGTAAACTGCCCACTTGGCAGTACATCAAGTG | |
| | | TATCATATGCCAAGTACGCCCCCTATTGACGTCAAT | |
| | | GACGGTAAATGGCCCGCCTGGCATTATGCCCAGTAC | |
| | | ATGACCTTATGGGACTTTCCTACTTGGCAGTACATC | |
| | | TACGTATTAGTCATCGCTATTACCATGGGTCGAGGT | |
| | | GAGCCCCACGTTCTGCTTCACTCTCCCCATCTCCCCC | |
| | | CCCTCCCCACCCCCAATTTTGTATTTATTTATTTTTT | |
| | | AATTATTTTGTGCAGCGATGGGGGCGGGGGGGGGG | |
| | | GGGGCGCGCGCCAGGCGGGGCGGGGCGGGGCGAG | |
| | | GGGCGGGGCGGGGCGAGGCGGAGAGGTGCGGCGG | |
| | | CAGCCAATCAGAGCGGCGCGCTCCGAAAGTTTCCTT | |
| | | TTATGGCGAGGCGGCGGCGGCGGCGGCCCTATAAA | |
| | | AAGCGAAGCGCGCGGCGGGCGGGAGTCGCTGCGTT | |
| | | GCCTTCGCCCCGTGCCCCGCTCCGCGCCGCCTCGCG | |
| | | CCGCCCGCCCCGGCTCTGACTGACCGCGTTACTCCC | |
| | | ACAGGTGAGCGGGCGGGACGGCCCTTCTCCTCCGG | |
| | | GCTGTAATTAGCGCTTGGTTTAATGACGGCTCGTTT | |
| | | CTTTTCTGTGGCTGCGTGAAAGCCTTAAAGGGCTCC | |
| | | GGGAGGGCCCTTTGTGCGGGGGGGAGCGGCTCGGG | |
| | | GGGTGCGTGCGTGTGTGTGTGCGTGGGGAGCGCCG | |
| | | CGTGCGGCCCGCGCTGCCCGGCGGCTGTGAGCGCTG | |
| | | CGGGCGCGGCGCGGGGCTTTGTGCGCTCCGCGTGTG | |
| | | CGCGAGGGGAGCGCGGCCGGGGGCGGTGCCCCGCG | |
| | | GTGCGGGGGGGCTGCGAGGGGAACAAAGGCTGCGT | |
| | | GCGGGGTGTGTGCGTGGGGGGGTGAGCAGGGGGTG | |
| | | TGGGCGCGGCGGTCGGGCTGTAACCCCCCCCTGCAC | |
| | | CCCCCTCCCCGAGTTGCTGAGCACGGCCCGGCTTCG | |
| | | GGTGCGGGGCTCCGTGCGGGGCGTGGCGCGGGGCT | |
| | | CGCCGTGCCGGGCGGGGGGTGGCGGCAGGTGGGGG | |
| | | TGCCGGGCGGGGCGGGGCCGCCTCGGGCCGGGGAG | |
| | | GGCTCGGGGGAGGGGCGCGGCGGCCCCGGAGCGCC | |
| | | GGCGGCTGTCGAGGCGCGGCGAGCCGCAGCCATTG | |
| | | CCTTTTATGGTAATCGTGCGAGAGGGCGCAGGGACT | |
| | | TCCTTTGTCCCAAATCTGGCGGAGCCGAAATCTGGG | |
| | | AGGCGCCGCCGCACCCCCTCTAGCGGGCGCGGGCG | |
| | | AAGCGGTGCGGCGCCGGCAGGAAGGAAATGGGCGG | |
| | | GGAGGGCCTTCGTGCGTCGCCGCGCCGCCGTCCCCT | |
| | | TCTCCATCTCCAGCCTCGGGGCTGCCGCAGGGGGAC | |
| | | GGCTGCCTTCGGGGGGGACGGGGCAGGGCGGGGTT | |
| | | CGGCTTCTGGCGTGTGACCGGCGGCTCTAGAGCCTC | |
| | | TGCTAACCATGTTCATGCCTTCTTCTTTTTCCTACAG | |
| | | GGGGGATCCGTTTATCTGCAGAATTCGCCCTTGACG | |
| | | TCGCCACCATGGACTGGACCTGGATCCTGTTCCTGG | |
| | | TGGCCGCCGCCACCAGGGTGCACAGCGGCATTCAT | |
| | | GTCTTCATTTTGGGCTGTTTCAGTGCAGGGCTTCCTA | |
| | | AAACAGAAGCCAACTGGGTGAATGTAATAAGTGAT | |
| | | TTGAAAAAAATTGAAGATCTTATTCAATCTATGCAT | |
| | | ATTGATGCTACTTTATATACGGAAAGTGATGTTCAC | |
| | | CCCAGTTGCAAAGTAACAGCAATGAAGTGCTTTCTC | |
| | | TTGGAGTTACAAGTTATTTCACTTGAGTCCGGAGAT | |
| | | GCAAGTATTCATGATACAGTAGAAAATCTGATCATC | |
| | | CTAGCAAACAACAGTTTGTCTTCTAATGGGAATGTA | |
| | | ACAGAATCTGGATGCAAAGAATGTGAGGAACTGGA | |
| | | GGAAAAAAATATTAAAGAATTTTTGCAGAGTTTTGT | |
| | | ACATATTGTCCAAATGTTCATCAACACTTCTAGCGG | |
| | | CGGCGGCAGCGGCGGCGGCGGCAGCGGCGGCGGCG | |
| | | GCAGCGGCGGCGGCGGCAGCGGCGGCGGCAGCCTG | |
| | | CAGATCACGTGCCCTCCCCCCATGTCCGTGGAACAC | |
| | | GCAGACATCTGGGTCAAGAGCTACAGCTTGTACTCC | |
| | | AGGGAGCGGTACATTTGTAACTCTGGTTTCAAGCGT | |
| | | AAAGCCGGCACGTCCAGCCTGACGGAGTGCGTGTT | |
| | | GAACAAGGCCACGAATGTCGCCCACTGGACAACCC | |
| | | CCAGTCTCAAATGCATTAGAGACCCTGCCCTGGTTC | |
| | | ACCAAAGGCCAGCGCCACCCTCCACAGTAACGACG | |
| | | GCAGGGGTGACCCCACAGCCAGAGAGCCTCTCCCC | |
| | | TTCTGGAAAAGAGCCCGCAGCTTCATCTCCCAGCTC | |
| | | AAACAACACAGCGGCCACAACAGCAGCTATTGTCC | |
| | | CGGGCTCCCAGCTGATGCCTTCAAAATCACCTTCCA | |
| | | CAGGAACCACAGAGATAAGCAGTCATGAGTCCTCC | |
| | | CACGGCACCCCCTCTCAGACAACAGCCAAGAACTG | |
| | | GGAACTCACAGCATCCGCCTCCCACCAGCCGCCAG | |
| | | GTGTGTATCCACAGGGCCACAGCGACACCACTGTG | |
| | | GCTATCTCCACGTCCACTGTCCTGCTGTGTGGGCTG | |
| | | AGCGCTGTGTCTCTCCTGGCATGCTACCTCAAGTCA | |
| | | AGGCAAACTCCCCCGCTGGCCAGCGTTGAAATGGA | |
| | | AGCCATGGAGGCTCTGCCGGTGACTTGGGGGACCA | |
| | | GCAGCAGAGATGAAGACTTGGAAAACTGCTCTCAC | |
| | | CACCTAGGAAGCGGAGCTACTAACTTCAGCCTGCTG | |
| | | AAGCAGGCTGGAGACGTGGAGGAGAACCCTGGACC | |
| | | TATGTCTCGCTCCGTTGCCTTAGCTGTGCTCGCGCTA | |
| | | CTCTCTCTTTCTGGATTAGAGGCTGTCATGGCGCCC | |
| | | CGAACCCTCTTCCTGGGTGGAGGCGGTTCAGGCGGA | |
| | | GGTGGCTCTGGCGGTGGCGGATCGATCCAGCGTACT | |
| | | CCAAAGATTCAGGTTTACTCACGTCATCCAGCAGAG | |
| | | AATGGAAAGTCAAATTTCCTGAATTGCTATGTGTCT | |
| | | GGGTTTCATCCATCCGACATTGAAGTTGACTTACTG | |
| | | AAGAATGGAGAGAGAATTGAAAAAGTGGAGCATTC | |
| | | AGACTTGTCTTTCAGCAAGGACTGGTCTTTCTATCTC | |
| | | TTGTACTACACTGAATTCACCCCCACTGAAAAAGAT | |
| | | GAGTATGCCTGCCGTGTGAACCATGTGACTTTGTCA | |
| | | CAGCCCAAGATAGTTAAGTGGGATCGAGACATGGG | |
| | | TGGTGGTGGTTCTGGTGGTGGTGGTTCTGGCGGCGG | |
| | | CGGCTCCGGTGGTGGTGGATCCGGCTCCCACTCCTT | |
| | | GAAGTATTTCCACACTTCCGTGTCCCGGCCCGGCCG | |
| | | CGGGGAGCCCCGCTTCATCTCTGTGGGCTACGTGGA | |
| | | CGACACCCAGTTCGTGCGCTTCGACAACGACGCCGC | |
| | | GAGTCCGAGGATGGTGCCGCGGGCGCCGTGGATGG | |
| | | AGCAGGAGGGGTCAGAGTATTGGGACCGGGAGACA | |
| | | CGGAGCGCCAGGGACACCGCACAGATTTTCCGAGT | |
| | | GAATCTGCGGACGCTGCGCGGCTACTACAATCAGA | |
| | | GCGAGGCCGGGTCTCACACCCTGCAGTGGATGCAT | |
| | | GGCTGCGAGCTGGGGCCCGACGGGCGCTTCCTCCGC | |
| | | GGGTATGAACAGTTCGCCTACGACGGCAAGGATTA | |
| | | TCTCACCCTGAATGAGGACCTGCGCTCCTGGACCGC | |
| | | GGTGGACACGGCGGCTCAGATCTCCGAGCAAAAGT | |
| | | CAAATGATGCCTCTGAGGCGGAGCACCAGAGAGCC | |
| | | TACCTGGAAGACACATGCGTGGAGTGGCTCCACAA | |
| | | ATACCTGGAGAAGGGGAAGGAGACGCTGCTTCACC | |
| | | TGGAGCCCCCAAAGACACACGTGACTCACCACCCC | |
| | | ATCTCTGACCATGAGGCCACCCTGAGGTGCTGGGCC | |
| | | CTGGGCTTCTACCCTGCGGAGATCACACTGACCTGG | |
| | | CAGCAGGATGGGGAGGGCCATACCCAGGACACGGA | |
| | | GCTCGTGGAGACCAGGCCTGCAGGGGATGGAACCT | |
| | | TCCAGAAGTGGGCAGCTGTGGTGGTGCCTTCTGGAG | |
| | | AGGAGCAGAGATACACGTGCCATGTGCAGCATGAG | |
| | | GGGCTACCCGAGCCCGTCACCCTGAGATGGAAGCC | |
| | | GGCTTCCCAGCCCACCATCCCCATCGTGGGCATCAT | |
| | | TGCTGGCCTGGTTCTCCTTGGATCTGTGGTCTCTGGA | |
| | | GCTGTGGTTGCTGCTGTGATATGGAGGAAGAAGAG | |
| | | CTCAGGTGGAAAAGGAGGGAGCTACTCTAAGGCTG | |
| | | AGTGGAGCGACAGTGCCCAGGGGTCTGAGTCTCAC | |
| | | AGCTTGTAATGATAGCCGCTGATCAGCCTCGACTGT | |
| | | GCCTTCTAGTTGCCAGCCATCTGTTGTTTGCCCCTCC | |
| | | CCCGTGCCTTCCTTGACCCTGGAAGGTGCCACTCCC | |
| | | ACTGTCCTTTCCTAATAAAATGAGGAAATTGCATCG | |
| | | CATTGTCTGAGTAGGTGTCATTCTATTCTGGGGGGT | |
| | | GGGGTGGGGCAGGACAGCAAGGGGGAGGATTGGG | |
| | | AAGACAATAGCAGGCATGCTGGGGATGCGGTGGGC | |
| | | TCTATGGGTCGACCCAGCGTGAGTCTCTCCTACCCT | |
| | | CCCGCTCTGGTCCTTCCTCTCCCGCTCTGCACCCTCT | |
| | | GTGGCCCTCGCTGTGCTCTCTCGCTCCGTGACTTCCC | |
| | | TTCTCCAAGTTCTCCTTGGTGGCCCGCCGTGGGGCT | |
| | | AGTCCAGGGCTGGATCTCGGGGAAGCGGCGGGGTG | |
| | | GCCTGGGAGTGGGGAAGGGGGTGCGCACCCGGGAC | |
| | | GCGCGCTACTTGCCCCTTTCGGCGGGGAGCAGGGG | |
| | | AGACCTTTGGCCTACGGCGACGGGAGGGTCGGGAC | |
| | | AAAGTTTAGGGCGTCGATAAGCGTCAGAGCGCCGA | |
| | | GGTTGGGGGAGGGTTTCTCTTCCGCTCTTTCGCGGG | |
| | | GCCTCTGGCTCCCCCAGCGCAGCTGGAGTGGGGGA | |
| | | CGGGTAGGCTCGTCCCAAAGGCGCGGCGCTGAGGT | |
| | | TTGTGAACGCGTGGAGGGGCGCTTGGGGTCTGGGG | |
| | | GAGGCGTCGCCCGGGTAAGCCTGTCTGCTGCGGCTC | |
| | | TGCTTCCCTTAGACTGGAGAGCTGTGGACTTCGTCT | |
| | | AGGCGCCCGCTAAGTTCGCATGTCCTAGCACCTCTG | |
| | | GGTCTATGTGGGGCCACACCGTGGGGAGGAAACAG | |
| | | CACGCGACGTTTGTAGAATGCTTGGCTGTGATACAA | |
| | | AGCGGTTTCGAATAATTAACTTATTTGTTCCCATCA | |
| | | CATGTCACTTTTAAAAAATTATAAGAACTACCCGTT | |
| | | ATTGACATCTTTCTGTGTGCCAAGGACTTTATGTGCT | |
| | | TTGCGTCATTTAATTTTGAAAACAGTTATCTTCCGCC | |
| | | ATAGATAACTACTATGGTTATCTTCTGGTAACCACG | |
| | | TGCGGACCGAGGCTGCAGCGTCGTCCTCCCTAGGAA | |
| | | CCCCTAGTGATGGAGTTGGCCACTCCCTCTCTGCGC | |
| | | GCTCGCTCGCTCACTGAGGCCGGGCGACCAAAGGT | |
| | | CGCCCGACGCCCGGGCTTTGCCCGGGCGGCCTCAGT | |
| | | GAGCGAGCGAGCGCGCAGCTGCCTGCAGGGGCGCC | |
| | | TGATGCGGTATTTTCTCCTTACGCATCTGTGCGGTAT | |
| | | TTCACACCGCATACGTCAAAGCAACCATAGTACGCG | |
| | | CCCTGTAGCGGCGCATTAAGCGCGGCGGGTGTGGT | |
| | | GGTTACGCGCAGCGTGACCGCTACACTTGCCAGCGC | |
| | | CCTAGCGCCCGCTCCTTTCGCTTTCTTCCCTTCCTTT | |
| | | CTCGCCACGTTCGCCGGCTTTCCCCGTCAAGCTCTA | |
| | | AATCGGGGGCTCCCTTTAGGGTTCCGATTTAGTGCT | |
| | | TTACGGCACCTCGACCCCAAAAAACTTGATTTGGGT | |
| | | GATGGTTCACGTAGTGGGCCATCGCCCTGATAGACG | |
| | | GTTTTTCGCCCTTTGACGTTGGAGTCCACGTTCTTTA | |
| | | ATAGTGGACTCTTGTTCCAAACTGGAACAACACTCA | |
| | | ACCCTATCTCGGGCTATTCTTTTGATTTATAAGGGAT | |
| | | TTTGCCGATTTCGGCCTATTGGTTAAAAAATGAGCT | |
| | | GATTTAACAAAAATTTAACGCGAATTTTAACAAAAT | |
| | | ATTAACGTTTACAATTTTATGGTGCACTCTCAGTAC | |
| | | AATCTGCTCTGATGCCGCATAGTTAAGCCAGCCCCG | |
| | | ACACCCGCCAACACCCGCTGACGCGCCCTGACGGG | |
| | | CTTGTCTGCTCCCGGCATCCGCTTACAGACAAGCTG | |
| | | TGACCGTCTCCGGGAGCTGCATGTGTCAGAGGTTTT | |
| | | CACCGTCATCACCGAAACGCGCGAGACGAAAGGGC | |
| | | CTCGTGATACGCCTATTTTTATAGGTTAATGTCATG | |
| | | AACAATAAAACTGTCTGCTTACATAAACAGTAATAC | |
| | | AAGGGGTGTTATGAGCCATATTCAACGGGAAACGT | |
| | | CGAGGCCGCGATTAAATTCCAACATGGATGCTGATT | |
| | | TATATGGGTATAAATGGGCTCGCGATAATGTCGGGC | |
| | | AATCAGGTGCGACAATCTATCGCTTGTATGGGAAGC | |
| | | CCGATGCGCCAGAGTTGTTTCTGAAACATGGCAAAG | |
| | | GTAGCGTTGCCAATGATGTTACAGATGAGATGGTCA | |
| | | GACTAAACTGGCTGACGGAATTTATGCCTCTTCCGA | |
| | | CCATCAAGCATTTTATCCGTACTCCTGATGATGCAT | |
| | | GGTTACTCACCACTGCGATCCCCGGAAAAACAGCAT | |
| | | TCCAGGTATTAGAAGAATATCCTGATTCAGGTGAAA | |
| | | ATATTGTTGATGCGCTGGCAGTGTTCCTGCGCCGGT | |
| | | TGCATTCGATTCCTGTTTGTAATTGTCCTTTTAACAG | |
| | | CGATCGCGTATTTCGTCTCGCTCAGGCGCAATCACG | |
| | | AATGAATAACGGTTTGGTTGATGCGAGTGATTTTGA | |
| | | TGACGAGCGTAATGGCTGGCCTGTTGAACAAGTCTG | |
| | | GAAAGAAATGCATAAACTTTTGCCATTCTCACCGGA | |
| | | TTCAGTCGTCACTCATGGTGATTTCTCACTTGATAAC | |
| | | CTTATTTTTGACGAGGGGAAATTAATAGGTTGTATT | |
| | | GATGTTGGACGAGTCGGAATCGCAGACCGATACCA | |
| | | GGATCTTGCCATCCTATGGAACTGCCTCGGTGAGTT | |
| | | TTCTCCTTCATTACAGAAACGGCTTTTTCAAAAATA | |
| | | TGGTATTGATAATCCTGATATGAATAAATTGCAGTT | |
| | | TCATTTGATGCTCGATGAGTTTTTCTAATCTCATGAC | |
| | | CAAAATCCCTTAACGTGAGTTTTCGTTCCACTGAGC | |
| | | GTCAGACCCCGTAGAAAAGATCAAAGGATCTTCTTG | |
| | | AGATCCTTTTTTTCTGCGCGTAATCTGCTGCTTGCAA | |
| | | ACAAAAAAACCACCGCTACCAGCGGTGGTTTGTTTG | |
| | | CCGGATCAAGAGCTACCAACTCTTTTTCCGAAGGTA | |
| | | ACTGGCTTCAGCAGAGCGCAGATACCAAATACTGTC | |
| | | CTTCTAGTGTAGCCGTAGTTAGGCCACCACTTCAAG | |
| | | AACTCTGTAGCACCGCCTACATACCTCGCTCTGCTA | |
| | | ATCCTGTTACCAGTGGCTGCTGCCAGTGGCGATAAG | |
| | | TCGTGTCTTACCGGGTTGGACTCAAGACGATAGTTA | |
| | | CCGGATAAGGCGCAGCGGTCGGGCTGAACGGGGGG | |
| | | TTCGTGCACACAGCCCAGCTTGGAGCGAACGACCTA | |
| | | CACCGAACTGAGATACCTACAGCGTGAGCTATGAG | |
| | | AAAGCGCCACGCTTCCCGAAGGGAGAAAGGCGGAC | |
| | | AGGTATCCGGTAAGCGGCAGGGTCGGAACAGGAGA | |
| | | GCGCACGAGGGAGCTTCCAGGGGGAAACGCCTGGT | |
| | | ATCTTTATAGTCCTGTCGGGTTTCGCCACCTCTGACT | |
| | | TGAGCGTCGATTTTTGTGATGCTCGTCAGGGGGGCG | |
| | | GAGCCTATGGAAAAACGCCAGCAACGCGGCCTTTTT | |
| | | ACGGTTCCTGGCCTTTTGCTGGCCTTTTGCTCACATG | |
| | | T | |
| Signal | CAR signal | MLLLVTSLLLCELPHPAFLLIP | 68 |
| Peptide 1 | peptide | | |
| Signal | CAR signal | MALPVTALLLPLALLLHAARP | 69 |
| Peptide 2 | peptide | | |
| Anti- | Coding sequence | atggcgcttccggtgacagcactgctcctccccttggcgctgttgctccacgcagcaa | 70 |
| BCMA | | ggccgcaggtgcagctggtgcagagcggagccgagctcaagaagcccggagcct | |
| CAR | | ccgtgaaggtgagctgcaaggccagcggcaacaccctgaccaactacgtgatccac | |
| | | tgggtgagacaagcccccggccaaaggctggagtggatgggctacatcctgcccta | |
| | | caacgacctgaccaagtacagccagaagttccagggcagggtgaccatcaccagg | |
| | | gataagagcgcctccaccgcctatatggagctgagcagcctgaggagcgaggacac | |
| | | cgctgtgtactactgtacaaggtgggactgggacggcttctttgacccctggggccag | |
| | | ggcacaacagtgaccgtcagcagcggcggcggaggcagcggcggcggcggcag | |
| | | cggcggaggcggaagcgaaatcgtgatgacccagagccccgccacactgagcgtg | |
| | | agccctggcgagagggccagcatctcctgcagggctagccaaagcctggtgcaca | |
| | | gcaacggcaacacccacctgcactggtaccagcagagacccggacaggctcccag | |
| | | gctgctgatctacagcgtgagcaacaggttctccgaggtgcctgccaggtttagcggc | |
| | | agcggaagcggcaccgactttaccctgaccatcagcagcgtggagtccgaggactt | |
| | | cgccgtgtattactgcagccagaccagccacatcccttacaccttcggcggcggcac | |
| | | caagctggagatcaaaagtgctgctgcctttgtcccggtatttctcccagccaaaccga | |
| | | ccacgactcccgccccgcgccctccgacacccgctcccaccatcgcctctcaacctc | |
| | | ttagtcttcgccccgaggcatgccgacccgccgccgggggtgctgttcatacgaggg | |
| | | gcttggacttcgcttgtgatatttacatttgggctccgttggcgggtacgtgcggcgtcct | |
| | | tttgttgtcactcgttattactttgtattgtaatcacaggaatcgcaaacggggcagaaag | |
| | | aaactcctgtatatattcaaacaaccatttatgagaccagtacaaactactcaagaggaa | |
| | | gatggctgtagctgccgatttccagaagaagaagaaggaggatgtgaactgcgagtg | |
| | | aagttttcccgaagcgcagacgctccggcatatcagcaaggacagaatcagctgtata | |
| | | acgaactgaatttgggacgccgcgaggagtatgacgtgcttgataaacgccggggg | |
| | | agagacccggaaatggggggtaaaccccgaagaaagaatccccaagaaggactct | |
| | | acaatgaactccagaaggataagatggcggaggcctactcagaaataggtatgaag | |
| | | ggcgaacgacgacggggaaaaggtcacgatggcctctaccaagggttgagtacgg | |
| | | caaccaaagatacgtacgatgcactgcatatgcaggccctgcctcccaga | |
| Anti- | scFV for anti- | caggtgcagctggtgcagagcggagccgagctcaagaagcccggagcctccgtga | 71 |
| BCMA | BCMA CAR | aggtgagctgcaaggccagcggcaacaccctgaccaactacgtgatccactgggtg | |
| scFv | | agacaagcccccggccaaaggctggagtggatgggctacatcctgccctacaacga | |
| | | cctgaccaagtacagccagaagttccagggcagggtgaccatcaccagggataaga | |
| | | gcgcctccaccgcctatatggagctgagcagcctgaggagcgaggacaccgctgtg | |
| | | tactactgtacaaggtgggactgggacggcttctttgacccctggggccagggcaca | |
| | | acagtgaccgtcagcagcggcggcggaggcagcggcggcggcggcagcggcgg | |
| | | aggcggaagcgaaatcgtgatgacccagagccccgccacactgagcgtgagccct | |
| | | ggcgagagggccagcatctcctgcagggctagccaaagcctggtgcacagcaacg | |
| | | gcaacacccacctgcactggtaccagcagagacccggacaggctcccaggctgctg | |
| | | atctacagcgtgagcaacaggttctccgaggtgcctgccaggtttagcggcagcgga | |
| | | agcggcaccgactttaccctgaccatcagcagcgtggagtccgaggacttcgccgtg | |
| | | tattactgcagccagaccagccacatcccttacaccttcggcggcggcaccaagctg | |
| | | gagatcaaa | |
| CD8a | CD8a | FVPVFLPAKPTTTPAPRPPTPAPTIASQPLSLRPEACRPA | 72 |
| transmembrane | transmembrane | AGGAVHTRGLDFACDIYIWAPLAGTCGVLLLSLVITL | |
| domain | domain | YCNHRNR | |
| CD8a | CD8a | IYIWAPLAGTCGVLLLSLVITLY | 73 |
| transmembrane | transmembrane | | |
| domain | domain | | |
| Anti- | Amino acid | MALPVTALLLPLALLLHAARPQVQLVQSGAELKKPGA | 74 |
| BCMA | sequence | SVKVSCKASGNTLTNYVIHWVRQAPGQRLEWMGYIL | |
| CAR | | PYNDLTKYSQKFQGRVTITRDKSASTAYMELSSLRSE | |
| | | DTAVYYCTRWDWDGFFDPWGQGTTVTVSSGGGGSG | |
| | | GGGSGGGGSEIVMTQSPATLSVSPGERASISCRASQSL | |
| | | VHSNGNTHLHWYQQRPGQAPRLLIYSVSNRFSEVPAR | |
| | | FSGSGSGTDFTLTISSVESEDFAVYYCSQTSHIPYTFGG | |
| | | GTKLEIKSAAAFVPVFLPAKPTTTPAPRPPTPAPTIASQ | |
| | | PLSLRPEACRPAAGGAVHTRGLDFACDIYIWAPLAGT | |
| | | CGVLLLSLVITLYCNHRNRKRGRKKLLYIFKQPFMRP | |
| | | VQTTQEEDGCSCRFPEEEEGGCELRVKFSRSADAPAY | |
| | | QQGQNQLYNELNLGRREEYDVLDKRRGRDPEMGGKP | |
| | | RRKNPQEGLYNELQKDKMAEAYSEIGMKGERRRGKG | |
| | | HDGLYQGLSTATKDTYDALHMQALPPR | |
| HLA-E | Coding sequence | atgtctcgctccgttgccttagctgtgctcgcgctactctctctttctggattagaggctgt | 75 |
| Trimer | | catggcgccccgaaccctcttcctgggtggaggcggttcaggcggaggtggctctg | |
| | | gcggtggcggatcgatccagcgtactccaaagattcaggtttactcacgtcatccagc | |
| | | agagaatggaaagtcaaatttcctgaattgctatgtgtctgggtttcatccatccgacatt | |
| | | gaagttgacttactgaagaatggagagagaattgaaaaagtggagcattcagacttgt | |
| | | ctttcagcaaggactggtctttctatctcttgtactacactgaattcacccccactgaaaa | |
| | | agatgagtatgcctgccgtgtgaaccatgtgactttgtcacagcccaagatagttaagt | |
| | | gggatcgagacatgggtggtggtggttctggtggtggtggttctggcggcggcggct | |
| | | ccggtggtggtggatccggctcccactccttgaagtatttccacacttccgtgtcccgg | |
| | | cccggccgcggggagccccgcttcatctctgtgggctacgtggacgacacccagttc | |
| | | gtgcgcttcgacaacgacgccgcgagtccgaggatggtgccgcgggcgccgtgga | |
| | | tggagcaggaggggtcagagtattgggaccgggagacacggagcgccagggaca | |
| | | ccgcacagattttccgagtgaatctgcggacgctgcgcggctactacaatcagagcg | |
| | | aggccgggtctcacaccctgcagtggatgcatggctgcgagctggggcccgacgg | |
| | | gcgcttcctccgcgggtatgaacagttcgcctacgacggcaaggattatctcaccctg | |
| | | aatgaggacctgcgctcctggaccgcggtggacacggcggctcagatctccgagca | |
| | | aaagtcaaatgatgcctctgaggcggagcaccagagagcctacctggaagacacat | |
| | | gcgtggagtggctccacaaatacctggagaaggggaaggagacgctgcttcacctg | |
| | | gagcccccaaagacacacgtgactcaccaccccatctctgaccatgaggccaccctg | |
| | | aggtgctgggccctgggcttctaccctgcggagatcacactgacctggcagcaggat | |
| | | ggggagggccatacccaggacacggagctcgtggagaccaggcctgcaggggat | |
| | | ggaaccttccagaagtgggcagctgtggtggtgccttctggagaggagcagagatac | |
| | | acgtgccatgtgcagcatgaggggctacccgagcccgtcaccctgagatggaagcc | |
| | | ggcttcccagcccaccatccccatcgtgggcatcattgctggcctggttctccttggat | |
| | | ctgtggtctctggagctgtggttgctgctgtgatatggaggaagaagagctcaggtgg | |
| | | aaaaggagggagctactctaaggctgagtggagcgacagtgcccaggggtctgagt | |
| | | ctcacagcttg | |
| IL15/IL15 | Coding sequence | atggactggacctggatcctgttcctggtggccgccgccaccagggtgcacagcgg | 76 |
| Rα fusion | | cattcatgtcttcattttgggctgtttcagtgcagggcttcctaaaacagaagccaactgg | |
| protein | | gtgaatgtaataagtgatttgaaaaaaattgaagatcttattcaatctatgcatattgatgc | |
| | | tactttatatacggaaagtgatgttcaccccagttgcaaagtaacagcaatgaagtgctt | |
| | | tctcttggagttacaagttatttcacttgagtccggagatgcaagtattcatgatacagta | |
| | | gaaaatctgatcatcctagcaaacaacagtttgtcttctaatgggaatgtaacagaatct | |
| | | ggatgcaaagaatgtgaggaactggaggaaaaaaatattaaagaatttttgcagagttt | |
| | | tgtacatattgtccaaatgttcatcaacacttctagcggcggcggcagcggcggcggc | |
| | | ggcagcggcggcggcggcagcggcggcggcggcagcggcggcggcagcctgc | |
| | | agatcacgtgccctccccccatgtccgtggaacacgcagacatctgggtcaagagct | |
| | | acagcttgtactccagggagcggtacatttgtaactctggtttcaagcgtaaagccggc | |
| | | acgtccagcctgacggagtgcgtgttgaacaaggccacgaatgtcgcccactggac | |
| | | aacccccagtctcaaatgcattagagaccctgccctggttcaccaaaggccagcgcc | |
| | | accctccacagtaacgacggcaggggtgaccccacagccagagagcctctcccctt | |
| | | ctggaaaagagcccgcagcttcatctcccagctcaaacaacacagcggccacaaca | |
| | | gcagctattgtcccgggctcccagctgatgccttcaaaatcaccttccacaggaacca | |
| | | cagagataagcagtcatgagtcctcccacggcaccccctctcagacaacagccaag | |
| | | aactgggaactcacagcatccgcctcccaccagccgccaggtgtgtatccacaggg | |
| | | ccacagcgacaccactgtggctatctccacgtccactgtcctgctgtgtgggctgagc | |
| | | gctgtgtctctcctggcatgctacctcaagtcaaggcaaactcccccgctggccagcg | |
| | | ttgaaatggaagccatggaggctctgccggtgacttgggggaccagcagcagagat | |
| | | gaagacttggaaaactgctctcaccacctaggaagcgga | |
| IL15/IL15 | Coding sequence | atggactggacctggatcctgttcctggtggccgccgccaccagggtgcacagcgg | 77 |
| Rα-P2A- | | cattcatgtcttcattttgggctgtttcagtgcagggcttcctaaaacagaagccaactgg | |
| HLA-E | | gtgaatgtaataagtgatttgaaaaaaattgaagatcttattcaatctatgcatattgatgc | |
| trimer | | tactttatatacggaaagtgatgttcaccccagttgcaaagtaacagcaatgaagtgctt | |
| coding | | tctcttggagttacaagttatttcacttgagtccggagatgcaagtattcatgatacagta | |
| sequence | | gaaaatctgatcatcctagcaaacaacagtttgtcttctaatgggaatgtaacagaatct | |
| | | ggatgcaaagaatgtgaggaactggaggaaaaaaatattaaagaatttttgcagagttt | |
| | | tgtacatattgtccaaatgttcatcaacacttctagcggcggcggcagcggcggcggc | |
| | | ggcagcggcggcggcggcagcggcggcggcggcagcggcggcggcagcctgc | |
| | | agatcacgtgccctccccccatgtccgtggaacacgcagacatctgggtcaagagct | |
| | | acagcttgtactccagggagcggtacatttgtaactctggtttcaagcgtaaagccggc | |
| | | acgtccagcctgacggagtgcgtgttgaacaaggccacgaatgtcgcccactggac | |
| | | aacccccagtctcaaatgcattagagaccctgccctggttcaccaaaggccagcgcc | |
| | | accctccacagtaacgacggcaggggtgaccccacagccagagagcctctcccctt | |
| | | ctggaaaagagcccgcagcttcatctcccagctcaaacaacacagcggccacaaca | |
| | | gcagctattgtcccgggctcccagctgatgccttcaaaatcaccttccacaggaacca | |
| | | cagagataagcagtcatgagtcctcccacggcaccccctctcagacaacagccaag | |
| | | aactgggaactcacagcatccgcctcccaccagccgccaggtgtgtatccacaggg | |
| | | ccacagcgacaccactgtggctatctccacgtccactgtcctgctgtgtgggctgagc | |
| | | gctgtgtctctcctggcatgctacctcaagtcaaggcaaactcccccgctggccagcg | |
| | | ttgaaatggaagccatggaggctctgccggtgacttgggggaccagcagcagagat | |
| | | gaagacttggaaaactgctctcaccacctaggaagcggagctactaacttcagcctgc | |
| | | tgaagcaggctggagacgtggaggagaaccctggacctatgtctcgctccgttgcctt | |
| | | agctgtgctcgcgctactctctctttctggattagaggctgtcatggcgccccgaaccct | |
| | | cttcctgggtggaggcggttcaggcggaggtggctctggcggtggcggatcgatcc | |
| | | agcgtactccaaagattcaggtttactcacgtcatccagcagagaatggaaagtcaaat | |
| | | ttcctgaattgctatgtgtctgggtttcatccatccgacattgaagttgacttactgaagaa | |
| | | tggagagagaattgaaaaagtggagcattcagacttgtctttcagcaaggactggtctt | |
| | | tctatctcttgtactacactgaattcacccccactgaaaaagatgagtatgcctgccgtgt | |
| | | gaaccatgtgactttgtcacagcccaagatagttaagtgggatcgagacatgggtggt | |
| | | ggtggttctggtggtggtggttctggcggcggcggctccggtggtggtggatccggc | |
| | | tcccactccttgaagtatttccacacttccgtgtcccggcccggccgcggggagcccc | |
| | | gcttcatctctgtgggctacgtggacgacacccagttcgtgcgcttcgacaacgacgc | |
| | | cgcgagtccgaggatggtgccgcgggcgccgtggatggagcaggaggggtcaga | |
| | | gtattgggaccgggagacacggagcgccagggacaccgcacagattttccgagtga | |
| | | atctgcggacgctgcgcggctactacaatcagagcgaggccgggtctcacaccctg | |
| | | cagtggatgcatggctgcgagctggggcccgacgggcgcttcctccgcgggtatga | |
| | | acagttcgcctacgacggcaaggattatctcaccctgaatgaggacctgcgctcctgg | |
| | | accgcggtggacacggcggctcagatctccgagcaaaagtcaaatgatgcctctga | |
| | | ggcggagcaccagagagcctacctggaagacacatgcgtggagtggctccacaaat | |
| | | acctggagaaggggaaggagacgctgcttcacctggagcccccaaagacacacgt | |
| | | gactcaccaccccatctctgaccatgaggccaccctgaggtgctgggccctgggctt | |
| | | ctaccctgcggagatcacactgacctggcagcaggatggggagggccatacccag | |
| | | gacacggagctcgtggagaccaggcctgcaggggatggaaccttccagaagtggg | |
| | | cagctgtggtggtgccttctggagaggagcagagatacacgtgccatgtgcagcatg | |
| | | aggggctacccgagcccgtcaccctgagatggaagccggcttcccagcccaccatc | |
| | | cccatcgtgggcatcattgctggcctggttctccttggatctgtggtctctggagctgtg | |
| | | gttgctgctgtgatatggaggaagaagagctcaggtggaaaaggagggagctactct | |
| | | aaggctgagtggagcgacagtgcccaggggtctgagtctcacagcttg | |
| B2M-1 | gRNA target in | GCTACTCTCTCTTTCTGGCC | 78, |
| gRNA | B2M | | 187 |
| | PAM (TGG), | | |
| | spacer sequence | | |
| B2M-3 | gRNA target in | CGCGAGCACAGCTAAGGCCA | 79, |
| gRNA | B2M | | 188 |
| | PAM (CGG), | | |
| | spacer sequence | | |
| FAS Ex3 | gRNA target in | CTAGGGACTGCACAGTCAAT | 80, |
| T1 | FAS, spacer | | 189 |
| | sequence | | |
| CISH Ex1 | gRNA target in | TCGCCGCTGCCGCGGGGACA | 81, |
| T2 | CISH, spacer | | 190 |
| | sequence | | |
| CISH Ex1 | gRNA target in | GACATGGTCCTCTGCGTTCA | 82, |
| T18 | CISH, spacer | | 191 |
| | sequence | | |
| CISH Ex2 | gRNA target in | GTCCGCTCCACAGCCAGCAA | 83, |
| T1 | CISH, spacer | | 192 |
| | sequence | | |
| CISH Ex2 | gRNA target in | GTTCCAGGGACGGGGCCCAC | 84, |
| T2 | CISH, spacer | | 193 |
| | sequence | | |
| CISH Ex3 | gRNA target in | TCGGGCCTCGCTGGCCGTAA | 85, |
| T1 | CISH, spacer | | 194 |
| | sequence | | |
| CISH Ex3 | gRNA target in | CGTACTAAGAACGTGCCTTC | 86, |
| T2 | CISH, spacer | | 195 |
| | sequence | | |
| CISH Ex3 | gRNA target in | GGGTTCCATTACGGCCAGCG | 87, |
| T3 | CISH, spacer | | 196 |
| | sequence | | |
| CISH Ex3 | gRNA target in | CAGGTGTTGTCGGGCCTCGC | 88, |
| T5 | CISH, spacer | | 197 |
| | sequence | | |
| CISH Ex3 | gRNA target in | TACTCAATGCGTACATTGGT | 89, |
| T6 | CISH, spacer | | 198 |
| | sequence | | |
| CISH Ex3 | gRNA target in | AAGGCTGACCACATCCGGAA | 90, |
| T9 | CISH, spacer | | 199 |
| | sequence | | |
| CISH Ex3 | gRNA target in | TACATTGGTGGGGCCACGAG | 91, |
| T11 | CISH, spacer | | 200 |
| | sequence | | |
| CISH Ex3 | gRNA target in | CTGTCAGTGAAAACCACTCG | 92, |
| T14 | CISH, spacer | | 201 |
| | sequence | | |
| Regnase-1 | gRNA target in | GGTCATCGATGGGAGCAACG | 93, |
| Ex2 T1 | Regnase-1, | | 202 |
| | spacer sequence | | |
| Regnase-1 | gRNA target in | CACCACCCCGCGGGACTAGA | 94, |
| Ex2 T2 | Regnase-1, | | 203 |
| | spacer sequence | | |
| ZC3H12A_ | gRNA target in | GGTCTGGCGCTCCCGCTCGG | 95, |
| Segment | Regnase-1, | | 204 |
| 2 T3 | spacer sequence | | |
| Regnase-1 | gRNA target in | CCACCACCCCGCGGGACTAG | 96, |
| Ex2 T4 | Regnase-1, | | 205 |
| | spacer sequence | | |
| Regnase-1 | gRNA target in | TTAGGGGTGCCACCACCCCG | 97, |
| Ex2 T5 | Regnase-1, | | 206 |
| | spacer sequence | | |
| Regnase-1 | gRNA target in | TTCACACCATCACGACGCGT | 98, |
| Ex4 T1 | Regnase-1, | | 207 |
| | spacer sequence | | |
| ZC3H12A_ | gRNA target in | ACACCATCACGACGCGTGGG | 99, |
| Segment | Regnase-1, | | 208 |
| 4 T2 | spacer sequence | | |
| ZC3H12A_ | gRNA target in | CTACGAGTCTGACGGGATCG | 100, |
| Segment | Regnase-1, | | 209 |
| 4 T3 | spacer sequence | | |
| ZC3H12A_ | gRNA target in | ACGACGCGTGGGTGGCAAGC | 101, |
| Segment | Regnase-1, | | 210 |
| 4 T7 | spacer sequence | | |
| Brent_vL | Brentuximab | DIVLTQSPASLAVSLGQRATISCKASQSVDFDGDSYMN | 102 |
| | variable light | WYQQKPGQPPKVLIYAASNLESGIPARFSGSGSGTDFT | |
| | domain | LNIHPVEEEDAATYYCQQSNEDPWTFGGGTKLEIK | |
| Brent_vH | Brentuximab | QIQLQQSGPEVVKPGASVKISCKASGYTFTDYYITWV | 103 |
| | variable heavy | KQKPGQGLEWIGWIYPGSGNTKYNEKFKGKATLTVD | |
| | domain | TSSSTAFMQLSSLTSEDTAVYFCANYGNYWFAYWGQ | |
| | | GTQVTVSA | |
| 5F11_vL | 5F11 variable | DIQMTQSPTSLSASVGDRVTITCRASQGISSWLTWYQQ | 104 |
| | light domain | KPEKAPKSLIYAASSLQSGVPSRFSGSGSGTDFTLTISSL | |
| | | QPEDFATYYCQQYDSYPITFGQGTRLEIK | |
| 5F11_vH | 5F11 variable | QVQLQQWGAGLLKPSETLSLTCAVYGGSFSAYYWSW | 105 |
| | heavy domin | IRQPPGKGLEWIGDINHGGGTNYNPSLKSRVTISVDTS | |
| | | KNQFSLKLNSVTAADTAVYYCASLTAYWGQGSLVTV | |
| | | SS | |
| Brent | Coding sequence | cagatccagctgcagcagagcggccccgaggtggtgaagcccggcgccagcgtg | 106 |
| vH_vL | | aagatcagctgcaaggccagcggctacaccttcaccgactactacatcacctgggtg | |
| domain | | aagcagaagcccggccagggcctggagtggatcggctggatctaccccggcagcg | |
| | | gcaacaccaagtacaacgagaagttcaagggcaaggccaccctgaccgtggacac | |
| | | cagcagcagcaccgccttcatgcagctgagcagcctgaccagcgaggacaccgcc | |
| | | gtgtacttctgcgccaactacggcaactactggttcgcctactggggccagggcaccc | |
| | | aggtgaccgtgagcgccggcggcggcggcagcggcggcggcggcagcggcgg | |
| | | cggcggcagcgacatcgtgctgacccagagccccgccagcctggccgtgagcctg | |
| | | ggccagagagccaccatcagctgcaaggccagccagagcgtggacttcgacggcg | |
| | | acagctacatgaactggtaccagcagaagcccggccagccccccaaggtgctgatc | |
| | | tacgccgccagcaacctggagagcggcatccccgccagattcagcggcagcggca | |
| | | gcggcaccgacttcaccctgaacatccaccccgtggaggaggaggacgccgccac | |
| | | ctactactgccagcagagcaacgaggacccctggaccttcggcggcggcaccaag | |
| | | ctggagatcaag | |
| CD28 | Coding sequence | agcaagagaagcagactgctgcacagcgactacatgaacatgacccccagaagac | 107 |
| intracellular | | ccggccccaccagaaagcactaccagccctacgccccccccagagacttcgccgc | |
| domain | | ctacagaagc | |
| Anti-CD30 | Coding sequence | cagatccagctgcagcagagcggccccgaggtggtgaagcccggcgccagcgtg | 108 |
| CAR 4 | | aagatcagctgcaaggccagcggctacaccttcaccgactactacatcacctgggtg | |
| | | aagcagaagcccggccagggcctggagtggatcggctggatctaccccggcagcg | |
| | | gcaacaccaagtacaacgagaagttcaagggcaaggccaccctgaccgtggacac | |
| | | cagcagcagcaccgccttcatgcagctgagcagcctgaccagcgaggacaccgcc | |
| | | gtgtacttctgcgccaactacggcaactactggttcgcctactggggccagggcaccc | |
| | | aggtgaccgtgagcgccggcggcggcggcagcggcggcggcggcagcggcgg | |
| | | cggcggcagcgacatcgtgctgacccagagccccgccagcctggccgtgagcctg | |
| | | ggccagagagccaccatcagctgcaaggccagccagagcgtggacttcgacggcg | |
| | | acagctacatgaactggtaccagcagaagcccggccagccccccaaggtgctgatc | |
| | | tacgccgccagcaacctggagagcggcatccccgccagattcagcggcagcggca | |
| | | gcggcaccgacttcaccctgaacatccaccccgtggaggaggaggacgccgccac | |
| | | ctactactgccagcagagcaacgaggacccctggaccttcggcggcggcaccaag | |
| | | ctggagatcaagagcgccgccgccttcgtgcccgtgttcctgcccgccaagcccacc | |
| | | accacccccgcccccagaccccccacccccgcccccaccatcgccagccagcccc | |
| | | tgagcctgagacccgaggcctgcagacccgccgccggcggcgccgtgcacacca | |
| | | gaggcctggacttcgcctgcgacatctacatctgggcccccctggccggcacctgcg | |
| | | gcgtgctgctgctgagcctggtgatcaccctgtactgcaaccacagaaacagaagca | |
| | | agagaagcagactgctgcacagcgactacatgaacatgacccccagaagacccgg | |
| | | ccccaccagaaagcactaccagccctacgccccccccagagacttcgccgcctaca | |
| | | gaagcagagtgaagttcagcagaagcgccgacgcccccgcctaccagcagggcca | |
| | | gaaccagctgtacaacgagctgaacctgggcagaagagaggagtacgacgtgctg | |
| | | gacaagagaagaggcagagaccccgagatgggcggcaagcccagaagaaagaa | |
| | | cccccaggagggcctgtacaacgagctgcagaaggacaagatggccgaggcctac | |
| | | agcgagatcggcatgaagggcgagagaagaagaggcaagggccacgacggcct | |
| | | gtaccagggcctgagcaccgccaccaaggacacctacgacgccctgcacatgcag | |
| | | gccctgccccccaga | |
| Anti-CD30 | Amino acid | QIQLQQSGPEVVKPGASVKISCKASGYTFTDYYITWV | 109 |
| CAR 4 | sequence | KQKPGQGLEWIGWIYPGSGNTKYNEKFKGKATLTVD | |
| | | TSSSTAFMQLSSLTSEDTAVYFCANYGNYWFAYWGQ | |
| | | GTQVTVSAGGGGSGGGGSGGGGSDIVLTQSPASLAVS | |
| | | LGQRATISCKASQSVDFDGDSYMNWYQQKPGQPPKV | |
| | | LIYAASNLESGIPARFSGSGSGTDFTLNIHPVEEEDAAT | |
| | | YYCQQSNEDPWTFGGGTKLEIKSAAAFVPVFLPAKPT | |
| | | TTPAPRPPTPAPTIASQPLSLRPEACRPAAGGAVHTRGL | |
| | | DFACDIYIWAPLAGTCGVLLLSLVITLYCNHRNRSKRS | |
| | | RLLHSDYMNMTPRRPGPTRKHYQPYAPPRDFAAYRS | |
| | | RVKFSRSADAPAYQQGQNQLYNELNLGRREEYDVLD | |
| | | KRRGRDPEMGGKPRRKNPQEGLYNELQKDKMAEAY | |
| | | SEIGMKGERRRGKGHDGLYQGLSTATKDTYDALHMQ | |
| | | ALPPR | |
| (CIITA)- | 11,265 bp | agaatctgctcagactccttcagtttacatattaggaaactgaggcacagaaaggagc | 110 |
| anti-CD30 | | agagacttgctcaagtccacccaaagcagtagagcattgtggttaaatgcaggacttc | |
| CAR 4- | | agtcagactgtctgggttcaaatcctggttccacttggacatgggtttccttacataaatc | |
| P2A-HLA- | | acttcacctctctgagcctcagttttctcatatgcaaagtgaggataataataataccttcc | |
| E donor | | ttacatggttactgatatgagtattaaatgtgccagctcatgtgcctggcgtataggaggt | |
| plasmid | | gctttataaaccttagctgttaccactcatggcattgccaaatgtgggacgggtctcctg | |
| | | actctctggtgtgagattgatggaatccacactttccagttcccttttctacctcctgggta | |
| | | tcttctcatatggttgtaagttccttggaggaagggaatgtggcttgctctctccaccacg | |
| | | ctgagcatataagaggtgctgaatgagcgcttttattcactcctctcatccccagccctc | |
| | | accagctgggagttgttgtaggtgtcaattttctgcctctttccaacaccctgtgaggtga | |
| | | ctgagcattgtcttccctcccaggcagctcacagtgtaagcttgtggacgatatcgaatt | |
| | | cgcacgacattgattattgactagttattaatagtaatcaattacggggtcattagttcata | |
| | | gcccatatatggagttccgcgttacataacttacggtaaatggcccgcctggctgaccg | |
| | | cccaacgacccccgcccattgacgtcaataatgacgtatgttcccatagtaacgccaat | |
| | | agggactttccattgacgtcaatgggtggactatttacggtaaactgcccacttggcagt | |
| | | acatcaagtgtatcatatgccaagtacgccccctattgacgtcaatgacggtaaatggc | |
| | | ccgcctggcattatgcccagtacatgaccttatgggactttcctacttggcagtacatct | |
| | | acgtattagtcatcgctattaccatgggtcgaggtgagccccacgttctgcttcactctc | |
| | | cccatctcccccccctccccacccccaattttgtatttatttattttttaattattttgtgcagc | |
| | | gatgggggcggggggggggggggcgcgcgccaggcggggcggggcggggcg | |
| | | aggggcggggcggggcgaggcggagaggtgcggcggcagccaatcagagcgg | |
| | | cgcgctccgaaagtttccttttatggcgaggcggcggcggcggcggccctataaaaa | |
| | | gcgaagcgcgcggcgggcgggagtcgctgcgttgccttcgccccgtgccccgctc | |
| | | cgcgccgcctcgcgccgcccgccccggctctgactgaccgcgttactcccacaggt | |
| | | gagcgggcgggacggcccttctcctccgggctgtaattagcgcttggtttaatgacgg | |
| | | ctcgtttcttttctgtggctgcgtgaaagccttaaagggctccgggagggccctttgtgc | |
| | | gggggggagcggctcggggggtgcgtgcgtgtgtgtgtgcgtggggagcgccgc | |
| | | gtgcggcccgcgctgcccggcggctgtgagcgctgcgggcgcggcgcggggcttt | |
| | | gtgcgctccgcgtgtgcgcgaggggagcgcggccgggggcggtgccccgcggtg | |
| | | cgggggggctgcgaggggaacaaaggctgcgtgcggggtgtgtgcgtggggggg | |
| | | tgagcagggggtgtgggcgcggcggtcgggctgtaacccccccctgcacccccctc | |
| | | cccgagttgctgagcacggcccggcttcgggtgcggggctccgtgcggggcgtgg | |
| | | cgcggggctcgccgtgccgggcggggggtggcggcaggtgggggtgccgggcg | |
| | | gggcggggccgcctcgggccggggagggctcgggggaggggcgcggcggccc | |
| | | cggagcgccggcggctgtcgaggcgcggcgagccgcagccattgccttttatggta | |
| | | atcgtgcgagagggcgcagggacttcctttgtcccaaatctggcggagccgaaatct | |
| | | gggaggcgccgccgcaccccctctagcgggcgcgggcgaagcggtgcggcgcc | |
| | | ggcaggaaggaaatgggcggggagggccttcgtgcgtcgccgcgccgccgtccc | |
| | | cttctccatctccagcctcggggctgccgcagggggacggctgccttcggggggga | |
| | | cggggcagggcggggttcggcttctggcgtgtgaccggcggctctagagcctctgct | |
| | | aaccatgttcatgccttcttctttttcctacaggggggatccgtttatctgcagaattcgcc | |
| | | cttgacgtcgccaccatggcgcttccggtgacagcactgctcctccccttggcgctgtt | |
| | | gctccacgcagcaaggccgcagatccagctgcagcagagcggccccgaggtggtg | |
| | | aagcccggcgccagcgtgaagatcagctgcaaggccagcggctacaccttcaccg | |
| | | actactacatcacctgggtgaagcagaagcccggccagggcctggagtggatcggc | |
| | | tggatctaccccggcagcggcaacaccaagtacaacgagaagttcaagggcaagg | |
| | | ccaccctgaccgtggacaccagcagcagcaccgccttcatgcagctgagcagcctg | |
| | | accagcgaggacaccgccgtgtacttctgcgccaactacggcaactactggttcgcct | |
| | | actggggccagggcacccaggtgaccgtgagcgccggcggcggcggcagcggc | |
| | | ggcggcggcagcggcggcggcggcagcgacatcgtgctgacccagagccccgc | |
| | | cagcctggccgtgagcctgggccagagagccaccatcagctgcaaggccagccag | |
| | | agcgtggacttcgacggcgacagctacatgaactggtaccagcagaagcccggcca | |
| | | gccccccaaggtgctgatctacgccgccagcaacctggagagcggcatccccgcca | |
| | | gattcagcggcagcggcagcggcaccgacttcaccctgaacatccaccccgtggag | |
| | | gaggaggacgccgccacctactactgccagcagagcaacgaggacccctggacct | |
| | | tcggcggcggcaccaagctggagatcaagagcgccgccgccttcgtgcccgtgttc | |
| | | ctgcccgccaagcccaccaccacccccgcccccagaccccccacccccgccccca | |
| | | ccatcgccagccagcccctgagcctgagacccgaggcctgcagacccgccgccgg | |
| | | cggcgccgtgcacaccagaggcctggacttcgcctgcgacatctacatctgggcccc | |
| | | cctggccggcacctgcggcgtgctgctgctgagcctggtgatcaccctgtactgcaa | |
| | | ccacagaaacagaagcaagagaagcagactgctgcacagcgactacatgaacatg | |
| | | acccccagaagacccggccccaccagaaagcactaccagccctacgcccccccca | |
| | | gagacttcgccgcctacagaagcagagtgaagttcagcagaagcgccgacgcccc | |
| | | cgcctaccagcagggccagaaccagctgtacaacgagctgaacctgggcagaaga | |
| | | gaggagtacgacgtgctggacaagagaagaggcagagaccccgagatgggcggc | |
| | | aagcccagaagaaagaacccccaggagggcctgtacaacgagctgcagaaggac | |
| | | aagatggccgaggcctacagcgagatcggcatgaagggcgagagaagaagaggc | |
| | | aagggccacgacggcctgtaccagggcctgagcaccgccaccaaggacacctacg | |
| | | acgccctgcacatgcaggccctgccccccagaggaagcggagctactaacttcagc | |
| | | ctgctgaagcaggctggagacgtggaggagaaccctggacctatgtctcgctccgtt | |
| | | gccttagctgtgctcgcgctactctctctttctggattagaggctgtcatggcgccccga | |
| | | accctcttcctgggtggaggcggttcaggcggaggtggctctggcggtggcggatc | |
| | | gatccagcgtactccaaagattcaggtttactcacgtcatccagcagagaatggaaag | |
| | | tcaaatttcctgaattgctatgtgtctgggtttcatccatccgacattgaagttgacttactg | |
| | | aagaatggagagagaattgaaaaagtggagcattcagacttgtctttcagcaaggact | |
| | | ggtctttctatctcttgtactacactgaattcacccccactgaaaaagatgagtatgcctg | |
| | | ccgtgtgaaccatgtgactttgtcacagcccaagatagttaagtgggatcgagacatg | |
| | | ggtggtggtggttctggtggtggtggttctggcggcggcggctccggtggtggtgga | |
| | | tccggctcccactccttgaagtatttccacacttccgtgtcccggcccggccgcgggg | |
| | | agccccgcttcatctctgtgggctacgtggacgacacccagttcgtgcgcttcgacaa | |
| | | cgacgccgcgagtccgaggatggtgccgcgggcgccgtggatggagcaggaggg | |
| | | gtcagagtattgggaccgggagacacggagcgccagggacaccgcacagattttcc | |
| | | gagtgaatctgcggacgctgcgcggctactacaatcagagcgaggccgggtctcac | |
| | | accctgcagtggatgcatggctgcgagctggggcccgacgggcgcttcctccgcgg | |
| | | gtatgaacagttcgcctacgacggcaaggattatctcaccctgaatgaggacctgcgc | |
| | | tcctggaccgcggtggacacggcggctcagatctccgagcaaaagtcaaatgatgc | |
| | | ctctgaggcggagcaccagagagcctacctggaagacacatgcgtggagtggctcc | |
| | | acaaatacctggagaaggggaaggagacgctgcttcacctggagcccccaaagac | |
| | | acacgtgactcaccaccccatctctgaccatgaggccaccctgaggtgctgggccct | |
| | | gggcttctaccctgcggagatcacactgacctggcagcaggatggggagggccata | |
| | | cccaggacacggagctcgtggagaccaggcctgcaggggatggaaccttccagaa | |
| | | gtgggcagctgtggtggtgccttctggagaggagcagagatacacgtgccatgtgca | |
| | | gcatgaggggctacccgagcccgtcaccctgagatggaagccggcttcccagccca | |
| | | ccatccccatcgtgggcatcattgctggcctggttctccttggatctgtggtctctggag | |
| | | ctgtggttgctgctgtgatatggaggaagaagagctcaggtggaaaaggagggagct | |
| | | actctaaggctgagtggagcgacagtgcccaggggtctgagtctcacagcttgtaatg | |
| | | atagccgctgatcagcctcgactgtgccttctagttgccagccatctgttgtttgcccctc | |
| | | ccccgtgccttccttgaccctggaaggtgccactcccactgtcctttcctaataaaatga | |
| | | ggaaattgcatcgcattgtctgagtaggtgtcattctattctggggggtggggtggggc | |
| | | aggacagcaagggggaggattgggaagacaatagcaggcatgctggggatgcggt | |
| | | gggctctatgggtcgactgaccagatggacctggctggagaagaagagattgagctc | |
| | | tactcaggtgggccctcctccctctggtctcttccggtatcccccacccctcagcttgct | |
| | | gtagagacggcaatcaggggaaattctggtccctgccctcccgtcagcaccacggac | |
| | | agctcccacgtctgtgggacgctctctgcagatggggatgatctcccagccctgcccc | |
| | | gcctctccctcgttccccaccagccctctttccagaaatttccttcttcatccaagggact | |
| | | tttcctcccagaacccgacacagacaccatcaactgcgaccagttcagcaggctgttg | |
| | | tgtgacatggaaggtgatgaagagaccagggaggcttatgccaatatcggtgaggaa | |
| | | gcacctgagcccagaaaaggacaatcaagggcaagagttctttgctgccacttgtcaa | |
| | | tatcacccattcatcatgagccacgtcagtcccctcccacagaaatcattgcaagggg | |
| | | gatgcggagcaatggctggaggaacggagactccagggaagagaggggagatgg | |
| | | aggccagtgggggaaataggccccttcactaatgaccaccaagaaaacaaaatctca | |
| | | tgtttacatcctccacctccatttctatacgcatttctgcttcttgctcttctgtccatcctttct | |
| | | acaaagcccataccatacacccctttcccttttcctcccagctccttagccaagctactct | |
| | | agtatttgtaataactagcatttactggatactcatagtatgctcattgctgtccggtaacc | |
| | | acgtgcggaccgggctccggtgcccgtcagtgggcagagcgcacatcgcccacag | |
| | | tccccgagaagttggggggaggggtcggcaattgaaccggtgcctagagaaggtg | |
| | | gcgcggggtaaactgggaaagtgatgtcgtgtactggctccgcctttttcccgagggt | |
| | | gggggagaaccgtatataagtgcagtagtcgccgtgaacgttctttttcgcaacgggtt | |
| | | tgccgccagaacacaggtaagtgccgtgtgtggttcccgcgggcctggcctctttacg | |
| | | ggttatggcccttgcgtgccttgaattacttccactggctgcagtacgtgattcttgatcc | |
| | | cgagcttcgggttggaagtgggtgggagagttcgaggccttgcgcttaaggagcccc | |
| | | ttcgcctcgtgcttgagttgaggcctggcctgggcgctggggccgccgcgtgcgaat | |
| | | ctggtggcaccttcgcgcctgtctcgctgctttcgataagtctctagccatttaaaattttt | |
| | | gatgacctgctgcgacgctttttttctggcaagatagtcttgtaaatgcgggccaagatc | |
| | | tgcacactggtatttcggtttttggggccgcgggcggcgacggggcccgtgcgtccc | |
| | | agcgcacatgttcggcgaggcggggcctgcgagcgcggccaccgagaatcggac | |
| | | gggggtagtctcaagctggccggcctgctctggtgcctggcctcgcgccgccgtgta | |
| | | tcgccccgccctgggcggcaaggctggcccggtcggcaccagttgcgtgagcgga | |
| | | aagatggccgcttcccggccctgctgcagggagctcaaaatggaggacgcggcgct | |
| | | cgggagagcgggcgggtgagtcacccacacaaaggaaaagggcctttccgtcctc | |
| | | agccgtcgcttcatgtgactccacggagtaccgggcgccgtccaggcacctcgatta | |
| | | gttctcgagcttttggagtacgtcgtctttaggttggggggaggggttttatgcgatgga | |
| | | gtttccccacactgagtgggtggagactgaagttaggccagcttggcacttgatgtaat | |
| | | tctccttggaatttgccctttttgagtttggatcttggttcattctcaagcctcagacagtgg | |
| | | ttcaaagtttttttcttccatttcaggtgtcgtgacttgacgtcgccaccatgaggatatttg | |
| | | ctgtctttatattcatgacctactggcatttgctgaacgcatttactgtcacggttcccaag | |
| | | gacctatatgtggtagagtatggtagcaatatgacaattgaatgcaaattcccagtaga | |
| | | aaaacaattagacctggctgcactaattgtctattgggaaatggaggataagaacattat | |
| | | tcaatttgtgcatggagaggaagacctgaaggttcagcatagtagctacagacagag | |
| | | ggcccggctgttgaaggaccagctctccctgggaaatgctgcacttcagatcacagat | |
| | | gtgaaattgcaggatgcaggggtgtaccgctgcatgatcagctatggtggtgccgact | |
| | | acaagcgaattactgtgaaagtcaatgccccatacaacaaaatcaaccaaagaattttg | |
| | | gttgtggatccagtcacctctgaacatgaactgacatgtcaggctgagggctacccca | |
| | | aggccgaagtcatctggacaagcagtgaccatcaagtcctgagtggtaagaccacca | |
| | | ccaccaattccaagagagaggagaaacttttcaatgtgaccagcacactgagaatcaa | |
| | | cacaacaactaatgagattttctactgcacttttaggagattagatcctgaggaaaacca | |
| | | tacagctgaattggtcatcccagaactacctctggcacatcctccaaatgaaaggactc | |
| | | acttggtaattctgggagccatcttattatgccttggtgtagcactgacattcatcttccgtt | |
| | | taagaaaagggagaatgatggatgtgaaaaaatgtggcatccaagatacaaactcaa | |
| | | agaagcaaagtgatacacatttggaggagacgtaaccgctgatcagcctcgaaactt | |
| | | gtttattgcagcttataatggttacaaataaagcaatagcatcacaaatttcacaaataaa | |
| | | gcatttttttcactgcattctagttgtggtttgtccaaactcatcaatgtatcttaggcgcct | |
| | | gatgcggtattttctccttacgcatctgtgcggtatttcacaccgcatacagtactgtcaa | |
| | | agcaaccatagtacgcgccctgtagcggcgcattaagcgcggcgggtgtggtggtta | |
| | | cgcgcagcgtgaccgctacacttgccagcgccctagcgcccgctcctttcgctttcttc | |
| | | ccttcctttctcgccacgttcgccggctttccccgtcaagctctaaatcgggggctccct | |
| | | ttagggttccgatttagtgctttacggcacctcgaccccaaaaaacttgatttgggtgat | |
| | | ggttcacgtagtgggccatcgccctgatagacggtttttcgccctttgacgttggagtcc | |
| | | acgttctttaatagtggactcttgttccaaactggaacaacactcaaccctatctcgggct | |
| | | attcttttgatttataagggattttgccgatttcggcctattggttaaaaaatgagctgattta | |
| | | acaaaaatttaacgcgaattttaacaaaatattaacgtttacaattttatggtgcactctca | |
| | | gtacaatctgctctgatgccgcatagttaagccagccccgacacccgccaacacccg | |
| | | ctgacgcgccctgacgggcttgtctgctcccggcatccgcttacagacaagctgtgac | |
| | | cgtctccgggagctgcatgtgtcagaggttttcaccgtcatcaccgaaacgcgcgaga | |
| | | cgaaagggcctcgtgatacgcctatttttataggttaatgtcatgaacaataaaactgtct | |
| | | gcttacataaacagtaatacaaggggtgttatgagccatattcaacgggaaacgtcga | |
| | | ggccgcgattaaattccaacatggatgctgatttatatgggtataaatgggctcgcgata | |
| | | atgtcgggcaatcaggtgcgacaatctatcgcttgtatgggaagcccgatgcgccag | |
| | | agttgtttctgaaacatggcaaaggtagcgttgccaatgatgttacagatgagatggtc | |
| | | agactaaactggctgacggaatttatgcctcttccgaccatcaagcattttatccgtactc | |
| | | ctgatgatgcatggttactcaccactgcgatccccggaaaaacagcattccaggtatta | |
| | | gaagaatatcctgattcaggtgaaaatattgttgatgcgctggcagtgttcctgcgccg | |
| | | gttgcattcgattcctgtttgtaattgtccttttaacagcgatcgcgtatttcgtctcgctca | |
| | | ggcgcaatcacgaatgaataacggtttggttgatgcgagtgattttgatgacgagcgta | |
| | | atggctggcctgttgaacaagtctggaaagaaatgcataaacttttgccattctcaccg | |
| | | gattcagtcgtcactcatggtgatttctcacttgataaccttatttttgacgaggggaaatt | |
| | | aataggttgtattgatgttggacgagtcggaatcgcagaccgataccaggatcttgcca | |
| | | tcctatggaactgcctcggtgagttttctccttcattacagaaacggctttttcaaaaatat | |
| | | ggtattgataatcctgatatgaataaattgcagtttcatttgatgctcgatgagtttttctaat | |
| | | ctcatgaccaaaatcccttaacgtgagttttcgttccactgagcgtcagaccccgtaga | |
| | | aaagatcaaaggatcttcttgagatcctttttttctgcgcgtaatctgctgcttgcaaacaa | |
| | | aaaaaccaccgctaccagcggtggtttgtttgccggatcaagagctaccaactctttttc | |
| | | cgaaggtaactggcttcagcagagcgcagataccaaatactgtccttctagtgtagcc | |
| | | gtagttaggccaccacttcaagaactctgtagcaccgcctacatacctcgctctgctaat | |
| | | cctgttaccagtggctgctgccagtggcgataagtcgtgtcttaccgggttggactcaa | |
| | | gacgatagttaccggataaggcgcagcggtcgggctgaacggggggttcgtgcaca | |
| | | cagcccagcttggagcgaacgacctacaccgaactgagatacctacagcgtgagct | |
| | | atgagaaagcgccacgcttcccgaagggagaaaggcggacaggtatccggtaagc | |
| | | ggcagggtcggaacaggagagcgcacgagggagcttccagggggaaacgcctgg | |
| | | tatctttatagtcctgtcgggtttcgccacctctgacttgagcgtcgatttttgtgatgctcg | |
| | | tcaggggggcggagcctatggaaaaacgccagcaacgcggcctttttacggttcctg | |
| | | gccttttgctggccttttgctcacatgtgcggccgcacgcgtcatatttatggggtatatg | |
| | | tgaatatttattacatgcatagaaggtataatgatcatgtcaggatatttgaggtatccaca | |
| | | tttgggattgtttaaagattaaatgaaatagtgttaaaagtatttaatatgcccttcaacaaa | |
| | | tgatgaggaaatctt | |
| 5F11_vH_ | | caggtgcagctgcagcagtggggcgccggcctgctgaagcccagcgagaccctga | 111 |
| vL domain | | gcctgacctgcgccgtgtacggcggcagcttcagcgcctactactggagctggatca | |
| | | gacagccccccggcaagggcctggagtggatcggcgacatcaaccacggcggcg | |
| | | gcaccaactacaaccccagcctgaagagcagagtgaccatcagcgtggacaccag | |
| | | caagaaccagttcagcctgaagctgaacagcgtgaccgccgccgacaccgccgtgt | |
| | | actactgcgccagcctgaccgcctactggggccagggcagcctggtgaccgtgagc | |
| | | agcggcggcggcggcagcggcggcggcggcagcggcggcggcggcagcgaca | |
| | | tccagatgacccagagccccaccagcctgagcgccagcgtgggcgacagagtgac | |
| | | catcacctgcagagccagccagggcatcagcagctggctgacctggtaccagcaga | |
| | | agcccgagaaggcccccaagagcctgatctacgccgccagcagcctgcagagcgg | |
| | | cgtgcccagcagattcagcggcagcggcagcggcaccgacttcaccctgaccatca | |
| | | gcagcctgcagcccgaggacttcgccacctactactgccagcagtacgacagctacc | |
| | | ccatcaccttcggccagggcaccagactggagatcaag | |
| Anti-CD30 | Coding sequence | caggtgcagctgcagcagtggggcgccggcctgctgaagcccagcgagaccctga | 112 |
| CAR 5 | | gcctgacctgcgccgtgtacggcggcagcttcagcgcctactactggagctggatca | |
| | | gacagccccccggcaagggcctggagtggatcggcgacatcaaccacggcggcg | |
| | | gcaccaactacaaccccagcctgaagagcagagtgaccatcagcgtggacaccag | |
| | | caagaaccagttcagcctgaagctgaacagcgtgaccgccgccgacaccgccgtgt | |
| | | actactgcgccagcctgaccgcctactggggccagggcagcctggtgaccgtgagc | |
| | | agcggcggcggcggcagcggcggcggcggcagcggcggcggcggcagcgaca | |
| | | tccagatgacccagagccccaccagcctgagcgccagcgtgggcgacagagtgac | |
| | | catcacctgcagagccagccagggcatcagcagctggctgacctggtaccagcaga | |
| | | agcccgagaaggcccccaagagcctgatctacgccgccagcagcctgcagagcgg | |
| | | cgtgcccagcagattcagcggcagcggcagcggcaccgacttcaccctgaccatca | |
| | | gcagcctgcagcccgaggacttcgccacctactactgccagcagtacgacagctacc | |
| | | ccatcaccttcggccagggcaccagactggagatcaagagcgccgccgccttcgtg | |
| | | cccgtgttcctgcccgccaagcccaccaccacccccgcccccagaccccccacccc | |
| | | cgcccccaccatcgccagccagcccctgagcctgagacccgaggcctgcagaccc | |
| | | gccgccggcggcgccgtgcacaccagaggcctggacttcgcctgcgacatctacat | |
| | | ctgggcccccctggccggcacctgcggcgtgctgctgctgagcctggtgatcaccct | |
| | | gtactgcaaccacagaaacagaaagagaggcagaaagaagctgctgtacatcttcaa | |
| | | gcagcccttcatgagacccgtgcagaccacccaggaggaggacggctgcagctgc | |
| | | agattccccgaggaggaggagggcggctgcgagctgagagtgaagttcagcagaa | |
| | | gcgccgacgcccccgcctaccagcagggccagaaccagctgtacaacgagctgaa | |
| | | cctgggcagaagagaggagtacgacgtgctggacaagagaagaggcagagaccc | |
| | | cgagatgggcggcaagcccagaagaaagaacccccaggagggcctgtacaacga | |
| | | gctgcagaaggacaagatggccgaggcctacagcgagatcggcatgaagggcga | |
| | | gagaagaagaggcaagggccacgacggcctgtaccagggcctgagcaccgccac | |
| | | caaggacacctacgacgccctgcacatgcaggccctgccccccaga | |
| Anti-CD30 | Amino acid | QVQLQQWGAGLLKPSETLSLTCAVYGGSFSAYYWSW | 113 |
| CAR 5 | sequence | IRQPPGKGLEWIGDINHGGGTNYNPSLKSRVTISVDTS | |
| | | KNQFSLKLNSVTAADTAVYYCASLTAYWGQGSLVTV | |
| | | SSGGGGSGGGGSGGGGSDIQMTQSPTSLSASVGDRVTI | |
| | | TCRASQGISSWLTWYQQKPEKAPKSLIYAASSLQSGVP | |
| | | SRFSGSGSGTDFTLTISSLQPEDFATYYCQQYDSYPITF | |
| | | GQGTRLEIKSAAAFVPVFLPAKPTTTPAPRPPTPAPTIA | |
| | | SQPLSLRPEACRPAAGGAVHTRGLDFACDIYIWAPLA | |
| | | GTCGVLLLSLVITLYCNHRNRKRGRKKLLYIFKQPFM | |
| | | RPVQTTQEEDGCSCRFPEEEEGGCELRVKFSRSADAPA | |
| | | YQQGQNQLYNELNLGRREEYDVLDKRRGRDPEMGG | |
| | | KPRRKNPQEGLYNELQKDKMAEAYSEIGMKGERRRG | |
| | | KGHDGLYQGLSTATKDTYDALHMQALPPR | |
| (CIITA) | 11,244 bp | atgtcaggatatttgaggtatccacatttgggattgtttaaagattaaatgaaatagtgtta | 114 |
| anti-CD30 | | aaagtatttaatatgcccttcaacaaatgatgaggaaatcttagaatctgctcagactcct | |
| CAR 5- | | tcagtttacatattaggaaactgaggcacagaaaggagcagagacttgctcaagtcca | |
| P2A-HLA- | | cccaaagcagtagagcattgtggttaaatgcaggacttcagtcagactgtctgggttca | |
| E donor | | aatcctggttccacttggacatgggtttccttacataaatcacttcacctctctgagcctca | |
| plasmid | | gttttctcatatgcaaagtgaggataataataataccttccttacatggttactgatatgag | |
| | | tattaaatgtgccagctcatgtgcctggcgtataggaggtgctttataaaccttagctgtt | |
| | | accactcatggcattgccaaatgtgggacgggtctcctgactctctggtgtgagattga | |
| | | tggaatccacactttccagttcccttttctacctcctgggtatcttctcatatggttgtaagtt | |
| | | ccttggaggaagggaatgtggcttgctctctccaccacgctgagcatataagaggtgc | |
| | | tgaatgagcgcttttattcactcctctcatccccagccctcaccagctgggagttgttgta | |
| | | ggtgtcaattttctgcctctttccaacaccctgtgaggtgactgagcattgtcttccctccc | |
| | | aggcagctcacagtgtaagcttgtggacgatatcgaattcgcacgacattgattattga | |
| | | ctagttattaatagtaatcaattacggggtcattagttcatagcccatatatggagttccgc | |
| | | gttacataacttacggtaaatggcccgcctggctgaccgcccaacgacccccgccca | |
| | | ttgacgtcaataatgacgtatgttcccatagtaacgccaatagggactttccattgacgt | |
| | | caatgggtggactatttacggtaaactgcccacttggcagtacatcaagtgtatcatatg | |
| | | ccaagtacgccccctattgacgtcaatgacggtaaatggcccgcctggcattatgccc | |
| | | agtacatgaccttatgggactttcctacttggcagtacatctacgtattagtcatcgctatt | |
| | | accatgggtcgaggtgagccccacgttctgcttcactctccccatctcccccccctccc | |
| | | cacccccaattttgtatttatttattttttaattattttgtgcagcgatgggggcggggggg | |
| | | gggggggcgcgcgccaggcggggcggggcggggcgaggggcggggcgggg | |
| | | cgaggcggagaggtgcggcggcagccaatcagagcggcgcgctccgaaagtttcc | |
| | | ttttatggcgaggcggcggcggcggcggccctataaaaagcgaagcgcgcggcgg | |
| | | gcgggagtcgctgcgttgccttcgccccgtgccccgctccgcgccgcctcgcgccg | |
| | | cccgccccggctctgactgaccgcgttactcccacaggtgagcgggcgggacggc | |
| | | ccttctcctccgggctgtaattagcgcttggtttaatgacggctcgtttcttttctgtggctg | |
| | | cgtgaaagccttaaagggctccgggagggccctttgtgcgggggggagcggctcg | |
| | | gggggtgcgtgcgtgtgtgtgtgcgtggggagcgccgcgtgcggcccgcgctgcc | |
| | | cggcggctgtgagcgctgcgggcgcggcgcggggctttgtgcgctccgcgtgtgc | |
| | | gcgaggggagcgcggccgggggcggtgccccgcggtgcgggggggctgcgag | |
| | | gggaacaaaggctgcgtgcggggtgtgtgcgtgggggggtgagcagggggtgtg | |
| | | ggcgcggcggtcgggctgtaacccccccctgcacccccctccccgagttgctgagc | |
| | | acggcccggcttcgggtgcggggctccgtgcggggcgtggcgcggggctcgccgt | |
| | | gccgggcggggggtggcggcaggtgggggtgccgggcggggcggggccgcct | |
| | | cgggccggggagggctcgggggaggggcgcggcggccccggagcgccggcgg | |
| | | ctgtcgaggcgcggcgagccgcagccattgccttttatggtaatcgtgcgagagggc | |
| | | gcagggacttcctttgtcccaaatctggcggagccgaaatctgggaggcgccgccgc | |
| | | accccctctagcgggcgcgggcgaagcggtgcggcgccggcaggaaggaaatgg | |
| | | gcggggagggccttcgtgcgtcgccgcgccgccgtccccttctccatctccagcctc | |
| | | ggggctgccgcagggggacggctgccttcgggggggacggggcagggcggggt | |
| | | tcggcttctggcgtgtgaccggcggctctagagcctctgctaaccatgttcatgccttct | |
| | | tctttttcctacaggggggatccgtttatctgcagaattcgcccttgacgtcgccaccatg | |
| | | gcgcttccggtgacagcactgctcctccccttggcgctgttgctccacgcagcaaggc | |
| | | cgcaggtgcagctgcagcagtggggcgccggcctgctgaagcccagcgagaccct | |
| | | gagcctgacctgcgccgtgtacggcggcagcttcagcgcctactactggagctggat | |
| | | cagacagccccccggcaagggcctggagtggatcggcgacatcaaccacggcgg | |
| | | cggcaccaactacaaccccagcctgaagagcagagtgaccatcagcgtggacacc | |
| | | agcaagaaccagttcagcctgaagctgaacagcgtgaccgccgccgacaccgccgt | |
| | | gtactactgcgccagcctgaccgcctactggggccagggcagcctggtgaccgtga | |
| | | gcagcggcggcggcggcagcggcggcggcggcagcggcggcggcggcagcg | |
| | | acatccagatgacccagagccccaccagcctgagcgccagcgtgggcgacagagt | |
| | | gaccatcacctgcagagccagccagggcatcagcagctggctgacctggtaccagc | |
| | | agaagcccgagaaggcccccaagagcctgatctacgccgccagcagcctgcagag | |
| | | cggcgtgcccagcagattcagcggcagcggcagcggcaccgacttcaccctgacc | |
| | | atcagcagcctgcagcccgaggacttcgccacctactactgccagcagtacgacagc | |
| | | taccccatcaccttcggccagggcaccagactggagatcaag | |
| | | agcgccgccgccttcgtgcccgtgttcctgcccgccaagcccaccaccacccccgc | |
| | | ccccagaccccccacccccgcccccaccatcgccagccagcccctgagcctgaga | |
| | | cccgaggcctgcagacccgccgccggcggcgccgtgcacaccagaggcctggac | |
| | | ttcgcctgcgacatctacatctgggcccccctggccggcacctgcggcgtgctgctgc | |
| | | tgagcctggtgatcaccctgtactgcaaccacagaaacagaaagagaggcagaaag | |
| | | aagctgctgtacatcttcaagcagcccttcatgagacccgtgcagaccacccaggag | |
| | | gaggacggctgcagctgcagattccccgaggaggaggagggcggctgcgagctg | |
| | | agagtgaagttcagcagaagcgccgacgcccccgcctaccagcagggccagaacc | |
| | | agctgtacaacgagctgaacctgggcagaagagaggagtacgacgtgctggacaa | |
| | | gagaagaggcagagaccccgagatgggcggcaagcccagaagaaagaaccccc | |
| | | aggagggcctgtacaacgagctgcagaaggacaagatggccgaggcctacagcga | |
| | | gatcggcatgaagggcgagagaagaagaggcaagggccacgacggcctgtacca | |
| | | gggcctgagcaccgccaccaaggacacctacgacgccctgcacatgcaggccctg | |
| | | ccccccagaggaagcggattcagcctgctgaagcaggctggagacgtggaggaga | |
| | | accctggacctatgtctcgctccgttgccttagctgtgctcgcgctactctctctttctgg | |
| | | attagaggctgtcatggcgccccgaaccctcttcctgggtggaggcggttcaggcgg | |
| | | aggtggctctggcggtggcggatcgatccagcgtactccaaagattcaggtttactca | |
| | | cgtcatccagcagagaatggaaagtcaaatttcctgaattgctatgtgtctgggtttcatc | |
| | | catccgacattgaagttgacttactgaagaatggagagagaattgaaaaagtggagca | |
| | | ttcagacttgtctttcagcaaggactggtctttctatctcttgtactacactgaattcacccc | |
| | | cactgaaaaagatgagtatgcctgccgtgtgaaccatgtgactttgtcacagcccaag | |
| | | atagttaagtgggatcgagacatgggtggtggtggttctggtggtggtggttctggcg | |
| | | gcggcggctccggtggtggtggatccggctcccactccttgaagtatttccacacttcc | |
| | | gtgtcccggcccggccgcggggagccccgcttcatctctgtgggctacgtggacga | |
| | | cacccagttcgtgcgcttcgacaacgacgccgcgagtccgaggatggtgccgcggg | |
| | | cgccgtggatggagcaggaggggtcagagtattgggaccgggagacacggagcg | |
| | | ccagggacaccgcacagattttccgagtgaatctgcggacgctgcgcggctactaca | |
| | | atcagagcgaggccgggtctcacaccctgcagtggatgcatggctgcgagctgggg | |
| | | cccgacgggcgcttcctccgcgggtatgaacagttcgcctacgacggcaaggattat | |
| | | ctcaccctgaatgaggacctgcgctcctggaccgcggtggacacggcggctcagat | |
| | | ctccgagcaaaagtcaaatgatgcctctgaggcggagcaccagagagcctacctgg | |
| | | aagacacatgcgtggagtggctccacaaatacctggagaaggggaaggagacgct | |
| | | gcttcacctggagcccccaaagacacacgtgactcaccaccccatctctgaccatga | |
| | | ggccaccctgaggtgctgggccctgggcttctaccctgcggagatcacactgacctg | |
| | | gcagcaggatggggagggccatacccaggacacggagctcgtggagaccaggcc | |
| | | tgcaggggatggaaccttccagaagtgggcagctgtggtggtgccttctggagagga | |
| | | gcagagatacacgtgccatgtgcagcatgaggggctacccgagcccgtcaccctga | |
| | | gatggaagccggcttcccagcccaccatccccatcgtgggcatcattgctggcctggt | |
| | | tctccttggatctgtggtctctggagctgtggttgctgctgtgatatggaggaagaagag | |
| | | ctcaggtggaaaaggagggagctactctaaggctgagtggagcgacagtgcccag | |
| | | gggtctgagtctcacagcttgtaatgatagccgctgatcagcctcgactgtgccttctag | |
| | | ttgccagccatctgttgtttgcccctcccccgtgccttccttgaccctggaaggtgccac | |
| | | tcccactgtcctttcctaataaaatgaggaaattgcatcgcattgtctgagtaggtgtcatt | |
| | | ctattctggggggtggggtggggcaggacagcaagggggaggattgggaagacaa | |
| | | tagcaggcatgctggggatgcggtgggctctatgggtcgactgaccagatggacctg | |
| | | gctggagaagaagagattgagctctactcaggtgggccctcctccctctggtctcttcc | |
| | | ggtatcccccacccctcagcttgctgtagagacggcaatcaggggaaattctggtccc | |
| | | tgccctcccgtcagcaccacggacagctcccacgtctgtgggacgctctctgcagat | |
| | | ggggatgatctcccagccctgccccgcctctccctcgttccccaccagccctctttcca | |
| | | gaaatttccttcttcatccaagggacttttcctcccagaacccgacacagacaccatcaa | |
| | | ctgcgaccagttcagcaggctgttgtgtgacatggaaggtgatgaagagaccaggga | |
| | | ggcttatgccaatatcggtgaggaagcacctgagcccagaaaaggacaatcaaggg | |
| | | caagagttctttgctgccacttgtcaatatcacccattcatcatgagccacgtcagtccc | |
| | | ctcccacagaaatcattgcaagggggatgcggagcaatggctggaggaacggaga | |
| | | ctccagggaagagaggggagatggaggccagtgggggaaataggccccttcacta | |
| | | atgaccaccaagaaaacaaaatctcatgtttacatcctccacctccatttctatacgcatt | |
| | | tctgcttcttgctcttctgtccatcctttctacaaagcccataccatacacccctttccctttt | |
| | | cctcccagctccttagccaagctactctagtatttgtaataactagcatttactggatactc | |
| | | atagtatgctcattgctgtccggtaaccacgtgcggaccgggctccggtgcccgtcag | |
| | | tgggcagagcgcacatcgcccacagtccccgagaagttggggggaggggtcggca | |
| | | attgaaccggtgcctagagaaggtggcgcggggtaaactgggaaagtgatgtcgtgt | |
| | | actggctccgcctttttcccgagggtgggggagaaccgtatataagtgcagtagtcgc | |
| | | cgtgaacgttctttttcgcaacgggtttgccgccagaacacaggtaagtgccgtgtgtg | |
| | | gttcccgcgggcctggcctctttacgggttatggcccttgcgtgccttgaattacttcca | |
| | | ctggctgcagtacgtgattcttgatcccgagcttcgggttggaagtgggtgggagagtt | |
| | | cgaggccttgcgcttaaggagccccttcgcctcgtgcttgagttgaggcctggcctgg | |
| | | gcgctggggccgccgcgtgcgaatctggtggcaccttcgcgcctgtctcgctgctttc | |
| | | gataagtctctagccatttaaaatttttgatgacctgctgcgacgctttttttctggcaagat | |
| | | agtcttgtaaatgcgggccaagatctgcacactggtatttcggtttttggggccgcggg | |
| | | cggcgacggggcccgtgcgtcccagcgcacatgttcggcgaggcggggcctgcg | |
| | | agcgcggccaccgagaatcggacgggggtagtctcaagctggccggcctgctctg | |
| | | gtgcctggcctcgcgccgccgtgtatcgccccgccctgggcggcaaggctggcccg | |
| | | gtcggcaccagttgcgtgagcggaaagatggccgcttcccggccctgctgcaggga | |
| | | gctcaaaatggaggacgcggcgctcgggagagcgggcgggtgagtcacccacac | |
| | | aaaggaaaagggcctttccgtcctcagccgtcgcttcatgtgactccacggagtaccg | |
| | | ggcgccgtccaggcacctcgattagttctcgagcttttggagtacgtcgtctttaggttg | |
| | | gggggaggggttttatgcgatggagtttccccacactgagtgggtggagactgaagtt | |
| | | aggccagcttggcacttgatgtaattctccttggaatttgccctttttgagtttggatcttgg | |
| | | ttcattctcaagcctcagacagtggttcaaagtttttttcttccatttcaggtgtcgtgactt | |
| | | gacgtcgccaccatgaggatatttgctgtctttatattcatgacctactggcatttgctga | |
| | | acgcatttactgtcacggttcccaaggacctatatgtggtagagtatggtagcaatatga | |
| | | caattgaatgcaaattcccagtagaaaaacaattagacctggctgcactaattgtctatt | |
| | | gggaaatggaggataagaacattattcaatttgtgcatggagaggaagacctgaaggt | |
| | | tcagcatagtagctacagacagagggcccggctgttgaaggaccagctctccctggg | |
| | | aaatgctgcacttcagatcacagatgtgaaattgcaggatgcaggggtgtaccgctgc | |
| | | atgatcagctatggtggtgccgactacaagcgaattactgtgaaagtcaatgccccata | |
| | | caacaaaatcaaccaaagaattttggttgtggatccagtcacctctgaacatgaactga | |
| | | catgtcaggctgagggctaccccaaggccgaagtcatctggacaagcagtgaccatc | |
| | | aagtcctgagtggtaagaccaccaccaccaattccaagagagaggagaaacttttca | |
| | | atgtgaccagcacactgagaatcaacacaacaactaatgagattttctactgcactttta | |
| | | ggagattagatcctgaggaaaaccatacagctgaattggtcatcccagaactacctct | |
| | | ggcacatcctccaaatgaaaggactcacttggtaattctgggagccatcttattatgcct | |
| | | tggtgtagcactgacattcatcttccgtttaagaaaagggagaatgatggatgtgaaaa | |
| | | aatgtggcatccaagatacaaactcaaagaagcaaagtgatacacatttggaggaga | |
| | | cgtaaccgctgatcagcctcgaaacttgtttattgcagcttataatggttacaaataaagc | |
| | | aatagcatcacaaatttcacaaataaagcatttttttcactgcattctagttgtggtttgtcc | |
| | | aaactcatcaatgtatcttaggcgcctgatgcggtattttctccttacgcatctgtgcggt | |
| | | atttcacaccgcatacagtactgtcaaagcaaccatagtacgcgccctgtagcggcgc | |
| | | attaagcgcggcgggtgtggtggttacgcgcagcgtgaccgctacacttgccagcgc | |
| | | cctagcgcccgctcctttcgctttcttcccttcctttctcgccacgttcgccggctttcccc | |
| | | gtcaagctctaaatcgggggctccctttagggttccgatttagtgctttacggcacctcg | |
| | | accccaaaaaacttgatttgggtgatggttcacgtagtgggccatcgccctgatagac | |
| | | ggtttttcgccctttgacgttggagtccacgttctttaatagtggactcttgttccaaactg | |
| | | gaacaacactcaaccctatctcgggctattcttttgatttataagggattttgccgatttcg | |
| | | gcctattggttaaaaaatgagctgatttaacaaaaatttaacgcgaattttaacaaaatatt | |
| | | aacgtttacaattttatggtgcactctcagtacaatctgctctgatgccgcatagttaagc | |
| | | cagccccgacacccgccaacacccgctgacgcgccctgacgggcttgtctgctccc | |
| | | ggcatccgcttacagacaagctgtgaccgtctccgggagctgcatgtgtcagaggtttt | |
| | | caccgtcatcaccgaaacgcgcgagacgaaagggcctcgtgatacgcctatttttata | |
| | | ggttaatgtcatgaacaataaaactgtctgcttacataaacagtaatacaaggggtgtta | |
| | | tgagccatattcaacgggaaacgtcgaggccgcgattaaattccaacatggatgctga | |
| | | tttatatgggtataaatgggctcgcgataatgtcgggcaatcaggtgcgacaatctatc | |
| | | gcttgtatgggaagcccgatgcgccagagttgtttctgaaacatggcaaaggtagcgt | |
| | | tgccaatgatgttacagatgagatggtcagactaaactggctgacggaatttatgcctct | |
| | | tccgaccatcaagcattttatccgtactcctgatgatgcatggttactcaccactgcgatc | |
| | | cccggaaaaacagcattccaggtattagaagaatatcctgattcaggtgaaaatattgtt | |
| | | gatgcgctggcagtgttcctgcgccggttgcattcgattcctgtttgtaattgtccttttaa | |
| | | cagcgatcgcgtatttcgtctcgctcaggcgcaatcacgaatgaataacggtttggttg | |
| | | atgcgagtgattttgatgacgagcgtaatggctggcctgttgaacaagtctggaaaga | |
| | | aatgcataaacttttgccattctcaccggattcagtcgtcactcatggtgatttctcacttg | |
| | | ataaccttatttttgacgaggggaaattaataggttgtattgatgttggacgagtcggaat | |
| | | cgcagaccgataccaggatcttgccatcctatggaactgcctcggtgagttttctccttc | |
| | | attacagaaacggctttttcaaaaatatggtattgataatcctgatatgaataaattgcagt | |
| | | ttcatttgatgctcgatgagtttttctaatctcatgaccaaaatcccttaacgtgagttttcgt | |
| | | tccactgagcgtcagaccccgtagaaaagatcaaaggatcttcttgagatcctttttttct | |
| | | gcgcgtaatctgctgcttgcaaacaaaaaaaccaccgctaccagcggtggtttgtttgc | |
| | | cggatcaagagctaccaactctttttccgaaggtaactggcttcagcagagcgcagat | |
| | | accaaatactgtccttctagtgtagccgtagttaggccaccacttcaagaactctgtagc | |
| | | accgcctacatacctcgctctgctaatcctgttaccagtggctgctgccagtggcgata | |
| | | agtcgtgtcttaccgggttggactcaagacgatagttaccggataaggcgcagcggtc | |
| | | gggctgaacggggggttcgtgcacacagcccagcttggagcgaacgacctacacc | |
| | | gaactgagatacctacagcgtgagctatgagaaagcgccacgcttcccgaagggag | |
| | | aaaggcggacaggtatccggtaagcggcagggtcggaacaggagagcgcacgag | |
| | | ggagcttccagggggaaacgcctggtatctttatagtcctgtcgggtttcgccacctct | |
| | | gacttgagcgtcgatttttgtgatgctcgtcaggggggcggagcctatggaaaaacgc | |
| | | cagcaacgcggcctttttacggttcctggccttttgctggccttttgctcacatgtgcggc | |
| | | cgcacgcgtcatatttatggggtatatgtgaatatttattacatgcatagaaggtataatg | |
| | | atc | |
| 5F11_vL_vH | | gacatccagatgacccagagccccaccagcctgagcgccagcgtgggcgacagag | 115 |
| domain | | tgaccatcacctgcagagccagccagggcatcagcagctggctgacctggtaccag | |
| | | cagaagcccgagaaggcccccaagagcctgatctacgccgccagcagcctgcaga | |
| | | gcggcgtgcccagcagattcagcggcagcggcagcggcaccgacttcaccctgac | |
| | | catcagcagcctgcagcccgaggacttcgccacctactactgccagcagtacgacag | |
| | | ctaccccatcaccttcggccagggcaccagactggagatcaagggcggcggcggc | |
| | | agcggcggcggcggcagcggcggcggcggcagccaggtgcagctgcagcagtg | |
| | | gggcgccggcctgctgaagcccagcgagaccctgagcctgacctgcgccgtgtac | |
| | | ggcggcagcttcagcgcctactactggagctggatcagacagccccccggcaagg | |
| | | gcctggagtggatcggcgacatcaaccacggcggcggcaccaactacaaccccag | |
| | | cctgaagagcagagtgaccatcagcgtggacaccagcaagaaccagttcagcctga | |
| | | agctgaacagcgtgaccgccgccgacaccgccgtgtactactgcgccagcctgacc | |
| | | gcctactggggccagggcagcctggtgaccgtg | |
| Anti-CD30 | Coding sequence | gacatccagatgacccagagccccaccagcctgagcgccagcgtgggcgacagag | 116 |
| CAR 6 | | tgaccatcacctgcagagccagccagggcatcagcagctggctgacctggtaccag | |
| | | cagaagcccgagaaggcccccaagagcctgatctacgccgccagcagcctgcaga | |
| | | gcggcgtgcccagcagattcagcggcagcggcagcggcaccgacttcaccctgac | |
| | | catcagcagcctgcagcccgaggacttcgccacctactactgccagcagtacgacag | |
| | | ctaccccatcaccttcggccagggcaccagactggagatcaagggcggcggcggc | |
| | | agcggcggcggcggcagcggcggcggcggcagccaggtgcagctgcagcagtg | |
| | | gggcgccggcctgctgaagcccagcgagaccctgagcctgacctgcgccgtgtac | |
| | | ggcggcagcttcagcgcctactactggagctggatcagacagccccccggcaagg | |
| | | gcctggagtggatcggcgacatcaaccacggcggcggcaccaactacaaccccag | |
| | | cctgaagagcagagtgaccatcagcgtggacaccagcaagaaccagttcagcctga | |
| | | agctgaacagcgtgaccgccgccgacaccgccgtgtactactgcgccagcctgacc | |
| | | gcctactggggccagggcagcctggtgaccgtgagcgccgccgccttcgtgcccgt | |
| | | gttcctgcccgccaagcccaccaccacccccgcccccagaccccccacccccgcc | |
| | | cccaccatcgccagccagcccctgagcctgagacccgaggcctgcagacccgccg | |
| | | ccggcggcgccgtgcacaccagaggcctggacttcgcctgcgacatctacatctgg | |
| | | gcccccctggccggcacctgcggcgtgctgctgctgagcctggtgatcaccctgtac | |
| | | tgcaaccacagaaacagaaagagaggcagaaagaagctgctgtacatcttcaagca | |
| | | gcccttcatgagacccgtgcagaccacccaggaggaggacggctgcagctgcagat | |
| | | tccccgaggaggaggagggcggctgcgagctgagagtgaagttcagcagaagcg | |
| | | ccgacgcccccgcctaccagcagggccagaaccagctgtacaacgagctgaacct | |
| | | gggcagaagagaggagtacgacgtgctggacaagagaagaggcagagaccccga | |
| | | gatgggcggcaagcccagaagaaagaacccccaggagggcctgtacaacgagct | |
| | | gcagaaggacaagatggccgaggcctacagcgagatcggcatgaagggcgagag | |
| | | aagaagaggcaagggccacgacggcctgtaccagggcctgagcaccgccaccaa | |
| | | ggacacctacgacgccctgcacatgcaggccctgccccccaga | |
| Anti-CD30 | Amino acid | DIQMTQSPTSLSASVGDRVTITCRASQGISSWLTWYQQ | 117 |
| CAR 6 | sequence | KPEKAPKSLIYAASSLQSGVPSRFSGSGSGTDFTLTISSL | |
| | | QPEDFATYYCQQYDSYPITFGQGTRLEIKGGGGSGGG | |
| | | GSGGGGSQVQLQQWGAGLLKPSETLSLTCAVYGGSFS | |
| | | AYYWSWIRQPPGKGLEWIGDINHGGGTNYNPSLKSRV | |
| | | TISVDTSKNQFSLKLNSVTAADTAVYYCASLTAYWGQ | |
| | | GSLVTVSAAAFVPVFLPAKPTTTPAPRPPTPAPTIASQP | |
| | | LSLRPEACRPAAGGAVHTRGLDFACDIYIWAPLAGTC | |
| | | GVLLLSLVITLYCNHRNRKRGRKKLLYIFKQPFMRPV | |
| | | QTTQEEDGCSCRFPEEEEGGCELRVKFSRSADAPAYQ | |
| | | QGQNQLYNELNLGRREEYDVLDKRRGRDPEMGGKPR | |
| | | RKNPQEGLYNELQKDKMAEAYSEIGMKGERRRGKGH | |
| | | DGLYQGLSTATKDTYDALHMQALPPR | |
| (CIITA) | 11,238 pb | tgcatagaaggtataatgatcatgtcaggatatttgaggtatccacatttgggattgtttaa | 118 |
| Anti-CD30 | | agattaaatgaaatagtgttaaaagtatttaatatgcccttcaacaaatgatgaggaaatc | |
| CAR 6- | | ttagaatctgctcagactccttcagtttacatattaggaaactgaggcacagaaaggag | |
| P2A-HLA- | | cagagacttgctcaagtccacccaaagcagtagagcattgtggttaaatgcaggactt | |
| E donor | | cagtcagactgtctgggttcaaatcctggttccacttggacatgggtttccttacataaat | |
| plasmid | | cacttcacctctctgagcctcagttttctcatatgcaaagtgaggataataataataccttc | |
| | | cttacatggttactgatatgagtattaaatgtgccagctcatgtgcctggcgtataggag | |
| | | gtgctttataaaccttagctgttaccactcatggcattgccaaatgtgggacgggtctcct | |
| | | gactctctggtgtgagattgatggaatccacactttccagttcccttttctacctcctgggt | |
| | | atcttctcatatggttgtaagttccttggaggaagggaatgtggcttgctctctccaccac | |
| | | gctgagcatataagaggtgctgaatgagcgcttttattcactcctctcatccccagccct | |
| | | caccagctgggagttgttgtaggtgtcaattttctgcctctttccaacaccctgtgaggtg | |
| | | actgagcattgtcttccctcccaggcagctcacagtgtaagcttgtggacgatatcgaa | |
| | | ttcgcacgacattgattattgactagttattaatagtaatcaattacggggtcattagttcat | |
| | | agcccatatatggagttccgcgttacataacttacggtaaatggcccgcctggctgacc | |
| | | gcccaacgacccccgcccattgacgtcaataatgacgtatgttcccatagtaacgcca | |
| | | atagggactttccattgacgtcaatgggtggactatttacggtaaactgcccacttggca | |
| | | gtacatcaagtgtatcatatgccaagtacgccccctattgacgtcaatgacggtaaatg | |
| | | gcccgcctggcattatgcccagtacatgaccttatgggactttcctacttggcagtacat | |
| | | ctacgtattagtcatcgctattaccatgggtcgaggtgagccccacgttctgcttcactct | |
| | | ccccatctcccccccctccccacccccaattttgtatttatttattttttaattattttgtgcag | |
| | | cgatgggggcggggggggggggggcgcgcgccaggcggggcggggcggggc | |
| | | gaggggcggggcggggcgaggcggagaggtgcggcggcagccaatcagagcg | |
| | | gcgcgctccgaaagtttccttttatggcgaggcggcggcggcggcggccctataaaa | |
| | | agcgaagcgcgcggcgggcgggagtcgctgcgttgccttcgccccgtgccccgct | |
| | | ccgcgccgcctcgcgccgcccgccccggctctgactgaccgcgttactcccacagg | |
| | | tgagcgggcgggacggcccttctcctccgggctgtaattagcgcttggtttaatgacg | |
| | | gctcgtttcttttctgtggctgcgtgaaagccttaaagggctccgggagggccctttgtg | |
| | | cgggggggagcggctcggggggtgcgtgcgtgtgtgtgtgcgtggggagcgccg | |
| | | cgtgcggcccgcgctgcccggcggctgtgagcgctgcgggcgcggcgcggggct | |
| | | ttgtgcgctccgcgtgtgcgcgaggggagcgcggccgggggcggtgccccgcggt | |
| | | gcgggggggctgcgaggggaacaaaggctgcgtgcggggtgtgtgcgtgggggg | |
| | | gtgagcagggggtgtgggcgcggcggtcgggctgtaacccccccctgcacccccc | |
| | | tccccgagttgctgagcacggcccggcttcgggtgcggggctccgtgcggggcgtg | |
| | | gcgcggggctcgccgtgccgggcggggggtggcggcaggtgggggtgccgggc | |
| | | ggggcggggccgcctcgggccggggagggctcgggggaggggcgcggcggcc | |
| | | ccggagcgccggcggctgtcgaggcgcggcgagccgcagccattgccttttatggt | |
| | | aatcgtgcgagagggcgcagggacttcctttgtcccaaatctggcggagccgaaatc | |
| | | tgggaggcgccgccgcaccccctctagcgggcgcgggcgaagcggtgcggcgcc | |
| | | ggcaggaaggaaatgggcggggagggccttcgtgcgtcgccgcgccgccgtccc | |
| | | cttctccatctccagcctcggggctgccgcagggggacggctgccttcggggggga | |
| | | cggggcagggcggggttcggcttctggcgtgtgaccggcggctctagagcctctgct | |
| | | aaccatgttcatgccttcttctttttcctacaggggggatccgtttatctgcagaattcgcc | |
| | | cttgacgtcgccaccatggcgcttccggtgacagcactgctcctccccttggcgctgtt | |
| | | gctccacgcagcaaggccggacatccagatgacccagagccccaccagcctgagc | |
| | | gccagcgtgggcgacagagtgaccatcacctgcagagccagccagggcatcagca | |
| | | gctggctgacctggtaccagcagaagcccgagaaggcccccaagagcctgatctac | |
| | | gccgccagcagcctgcagagcggcgtgcccagcagattcagcggcagcggcagc | |
| | | ggcaccgacttcaccctgaccatcagcagcctgcagcccgaggacttcgccacctac | |
| | | tactgccagcagtacgacagctaccccatcaccttcggccagggcaccagactgga | |
| | | gatcaagggcggcggcggcagcggcggcggcggcagcggcggcggcggcagc | |
| | | caggtgcagctgcagcagtggggcgccggcctgctgaagcccagcgagaccctga | |
| | | gcctgacctgcgccgtgtacggcggcagcttcagcgcctactactggagctggatca | |
| | | gacagccccccggcaagggcctggagtggatcggcgacatcaaccacggcggcg | |
| | | gcaccaactacaaccccagcctgaagagcagagtgaccatcagcgtggacaccag | |
| | | caagaaccagttcagcctgaagctgaacagcgtgaccgccgccgacaccgccgtgt | |
| | | actactgcgccagcctgaccgcctactggggccagggcagcctggtgaccgtgagc | |
| | | gccgccgccttcgtgcccgtgttcctgcccgccaagcccaccaccacccccgcccc | |
| | | cagaccccccacccccgcccccaccatcgccagccagcccctgagcctgagaccc | |
| | | gaggcctgcagacccgccgccggcggcgccgtgcacaccagaggcctggacttcg | |
| | | cctgcgacatctacatctgggcccccctggccggcacctgcggcgtgctgctgctga | |
| | | gcctggtgatcaccctgtactgcaaccacagaaacagaaagagaggcagaaagaag | |
| | | ctgctgtacatcttcaagcagcccttcatgagacccgtgcagaccacccaggaggag | |
| | | gacggctgcagctgcagattccccgaggaggaggagggcggctgcgagctgaga | |
| | | gtgaagttcagcagaagcgccgacgcccccgcctaccagcagggccagaaccagc | |
| | | tgtacaacgagctgaacctgggcagaagagaggagtacgacgtgctggacaagag | |
| | | aagaggcagagaccccgagatgggcggcaagcccagaagaaagaacccccagg | |
| | | agggcctgtacaacgagctgcagaaggacaagatggccgaggcctacagcgagat | |
| | | cggcatgaagggcgagagaagaagaggcaagggccacgacggcctgtaccagg | |
| | | gcctgagcaccgccaccaaggacacctacgacgccctgcacatgcaggccctgcc | |
| | | ccccagaggaagcggagctactaacttcagcctgctgaagcaggctggagacgtgg | |
| | | aggagaaccctggacctatgtctcgctccgttgccttagctgtgctcgcgctactctctc | |
| | | tttctggattagaggctgtcatggcgccccgaaccctcttcctgggtggaggcggttca | |
| | | ggcggaggtggctctggcggtggcggatcgatccagcgtactccaaagattcaggtt | |
| | | tactcacgtcatccagcagagaatggaaagtcaaatttcctgaattgctatgtgtctggg | |
| | | tttcatccatccgacattgaagttgacttactgaagaatggagagagaattgaaaaagt | |
| | | ggagcattcagacttgtctttcagcaaggactggtctttctatctcttgtactacactgaat | |
| | | tcacccccactgaaaaagatgagtatgcctgccgtgtgaaccatgtgactttgtcacag | |
| | | cccaagatagttaagtgggatcgagacatgggtggtggtggttctggtggtggtggtt | |
| | | ctggcggcggcggctccggtggtggtggatccggctcccactccttgaagtatttcca | |
| | | cacttccgtgtcccggcccggccgcggggagccccgcttcatctctgtgggctacgt | |
| | | ggacgacacccagttcgtgcgcttcgacaacgacgccgcgagtccgaggatggtgc | |
| | | cgcgggcgccgtggatggagcaggaggggtcagagtattgggaccgggagacac | |
| | | ggagcgccagggacaccgcacagattttccgagtgaatctgcggacgctgcgcggc | |
| | | tactacaatcagagcgaggccgggtctcacaccctgcagtggatgcatggctgcgag | |
| | | ctggggcccgacgggcgcttcctccgcgggtatgaacagttcgcctacgacggcaa | |
| | | ggattatctcaccctgaatgaggacctgcgctcctggaccgcggtggacacggcggc | |
| | | tcagatctccgagcaaaagtcaaatgatgcctctgaggcggagcaccagagagccta | |
| | | cctggaagacacatgcgtggagtggctccacaaatacctggagaaggggaaggag | |
| | | acgctgcttcacctggagcccccaaagacacacgtgactcaccaccccatctctgac | |
| | | catgaggccaccctgaggtgctgggccctgggcttctaccctgcggagatcacactg | |
| | | acctggcagcaggatggggagggccatacccaggacacggagctcgtggagacc | |
| | | aggcctgcaggggatggaaccttccagaagtgggcagctgtggtggtgccttctgga | |
| | | gaggagcagagatacacgtgccatgtgcagcatgaggggctacccgagcccgtca | |
| | | ccctgagatggaagccggcttcccagcccaccatccccatcgtgggcatcattgctg | |
| | | gcctggttctccttggatctgtggtctctggagctgtggttgctgctgtgatatggagga | |
| | | agaagagctcaggtggaaaaggagggagctactctaaggctgagtggagcgacag | |
| | | tgcccaggggtctgagtctcacagcttgtaatgatagccgctgatcagcctcgactgtg | |
| | | ccttctagttgccagccatctgttgtttgcccctcccccgtgccttccttgaccctggaag | |
| | | gtgccactcccactgtcctttcctaataaaatgaggaaattgcatcgcattgtctgagta | |
| | | ggtgtcattctattctggggggtggggtggggcaggacagcaagggggaggattgg | |
| | | gaagacaatagcaggcatgctggggatgcggtgggctctatgggtcgactgaccag | |
| | | atggacctggctggagaagaagagattgagctctactcaggtgggccctcctccctct | |
| | | ggtctcttccggtatcccccacccctcagcttgctgtagagacggcaatcaggggaaa | |
| | | ttctggtccctgccctcccgtcagcaccacggacagctcccacgtctgtgggacgctc | |
| | | tctgcagatggggatgatctcccagccctgccccgcctctccctcgttccccaccagc | |
| | | cctctttccagaaatttccttcttcatccaagggacttttcctcccagaacccgacacaga | |
| | | caccatcaactgcgaccagttcagcaggctgttgtgtgacatggaaggtgatgaaga | |
| | | gaccagggaggcttatgccaatatcggtgaggaagcacctgagcccagaaaaggac | |
| | | aatcaagggcaagagttctttgctgccacttgtcaatatcacccattcatcatgagccac | |
| | | gtcagtcccctcccacagaaatcattgcaagggggatgcggagcaatggctggagg | |
| | | aacggagactccagggaagagaggggagatggaggccagtgggggaaataggcc | |
| | | ccttcactaatgaccaccaagaaaacaaaatctcatgtttacatcctccacctccatttct | |
| | | atacgcatttctgcttcttgctcttctgtccatcctttctacaaagcccataccatacaccc | |
| | | ctttcccttttcctcccagctccttagccaagctactctagtatttgtaataactagcattta | |
| | | ctggatactcatagtatgctcattgctgtccggtaaccacgtgcggaccgggctccggt | |
| | | gcccgtcagtgggcagagcgcacatcgcccacagtccccgagaagttggggggag | |
| | | gggtcggcaattgaaccggtgcctagagaaggtggcgcggggtaaactgggaaagt | |
| | | gatgtcgtgtactggctccgcctttttcccgagggtgggggagaaccgtatataagtgc | |
| | | agtagtcgccgtgaacgttctttttcgcaacgggtttgccgccagaacacaggtaagtg | |
| | | ccgtgtgtggttcccgcgggcctggcctctttacgggttatggcccttgcgtgccttga | |
| | | attacttccactggctgcagtacgtgattcttgatcccgagcttcgggttggaagtgggt | |
| | | gggagagttcgaggccttgcgcttaaggagccccttcgcctcgtgcttgagttgaggc | |
| | | ctggcctgggcgctggggccgccgcgtgcgaatctggtggcaccttcgcgcctgtct | |
| | | cgctgctttcgataagtctctagccatttaaaatttttgatgacctgctgcgacgctttttttc | |
| | | tggcaagatagtcttgtaaatgcgggccaagatctgcacactggtatttcggtttttggg | |
| | | gccgcgggcggcgacggggcccgtgcgtcccagcgcacatgttcggcgaggcgg | |
| | | ggcctgcgagcgcggccaccgagaatcggacgggggtagtctcaagctggccggc | |
| | | ctgctctggtgcctggcctcgcgccgccgtgtatcgccccgccctgggcggcaagg | |
| | | ctggcccggtcggcaccagttgcgtgagcggaaagatggccgcttcccggccctgc | |
| | | tgcagggagctcaaaatggaggacgcggcgctcgggagagcgggcgggtgagtc | |
| | | acccacacaaaggaaaagggcctttccgtcctcagccgtcgcttcatgtgactccacg | |
| | | gagtaccgggcgccgtccaggcacctcgattagttctcgagcttttggagtacgtcgtc | |
| | | tttaggttggggggaggggttttatgcgatggagtttccccacactgagtgggtggag | |
| | | actgaagttaggccagcttggcacttgatgtaattctccttggaatttgccctttttgagttt | |
| | | ggatcttggttcattctcaagcctcagacagtggttcaaagtttttttcttccatttcaggtg | |
| | | tcgtgacttgacgtcgccaccatgaggatatttgctgtctttatattcatgacctactggc | |
| | | atttgctgaacgcatttactgtcacggttcccaaggacctatatgtggtagagtatggta | |
| | | gcaatatgacaattgaatgcaaattcccagtagaaaaacaattagacctggctgcacta | |
| | | attgtctattgggaaatggaggataagaacattattcaatttgtgcatggagaggaaga | |
| | | cctgaaggttcagcatagtagctacagacagagggcccggctgttgaaggaccagct | |
| | | ctccctgggaaatgctgcacttcagatcacagatgtgaaattgcaggatgcaggggtg | |
| | | taccgctgcatgatcagctatggtggtgccgactacaagcgaattactgtgaaagtcaa | |
| | | tgccccatacaacaaaatcaaccaaagaattttggttgtggatccagtcacctctgaac | |
| | | atgaactgacatgtcaggctgagggctaccccaaggccgaagtcatctggacaagca | |
| | | gtgaccatcaagtcctgagtggtaagaccaccaccaccaattccaagagagaggag | |
| | | aaacttttcaatgtgaccagcacactgagaatcaacacaacaactaatgagattttctac | |
| | | tgcacttttaggagattagatcctgaggaaaaccatacagctgaattggtcatcccaga | |
| | | actacctctggcacatcctccaaatgaaaggactcacttggtaattctgggagccatctt | |
| | | attatgccttggtgtagcactgacattcatcttccgtttaagaaaagggagaatgatgga | |
| | | tgtgaaaaaatgtggcatccaagatacaaactcaaagaagcaaagtgatacacatttg | |
| | | gaggagacgtaaccgctgatcagcctcgaaacttgtttattgcagcttataatggttaca | |
| | | aataaagcaatagcatcacaaatttcacaaataaagcatttttttcactgcattctagttgt | |
| | | ggtttgtccaaactcatcaatgtatcttaggcgcctgatgcggtattttctccttacgcatc | |
| | | tgtgcggtatttcacaccgcatacagtactgtcaaagcaaccatagtacgcgccctgta | |
| | | gcggcgcattaagcgcggcgggtgtggtggttacgcgcagcgtgaccgctacactt | |
| | | gccagcgccctagcgcccgctcctttcgctttcttcccttcctttctcgccacgttcgccg | |
| | | gctttccccgtcaagctctaaatcgggggctccctttagggttccgatttagtgctttacg | |
| | | gcacctcgaccccaaaaaacttgatttgggtgatggttcacgtagtgggccatcgccct | |
| | | gatagacggtttttcgccctttgacgttggagtccacgttctttaatagtggactcttgttc | |
| | | caaactggaacaacactcaaccctatctcgggctattcttttgatttataagggattttgc | |
| | | cgatttcggcctattggttaaaaaatgagctgatttaacaaaaatttaacgcgaattttaa | |
| | | caaaatattaacgtttacaattttatggtgcactctcagtacaatctgctctgatgccgcat | |
| | | agttaagccagccccgacacccgccaacacccgctgacgcgccctgacgggcttgt | |
| | | ctgctcccggcatccgcttacagacaagctgtgaccgtctccgggagctgcatgtgtc | |
| | | agaggttttcaccgtcatcaccgaaacgcgcgagacgaaagggcctcgtgatacgcc | |
| | | tatttttataggttaatgtcatgaacaataaaactgtctgcttacataaacagtaatacaag | |
| | | gggtgttatgagccatattcaacgggaaacgtcgaggccgcgattaaattccaacatg | |
| | | gatgctgatttatatgggtataaatgggctcgcgataatgtcgggcaatcaggtgcgac | |
| | | aatctatcgcttgtatgggaagcccgatgcgccagagttgtttctgaaacatggcaaag | |
| | | gtagcgttgccaatgatgttacagatgagatggtcagactaaactggctgacggaattt | |
| | | atgcctcttccgaccatcaagcattttatccgtactcctgatgatgcatggttactcacca | |
| | | ctgcgatccccggaaaaacagcattccaggtattagaagaatatcctgattcaggtga | |
| | | aaatattgttgatgcgctggcagtgttcctgcgccggttgcattcgattcctgtttgtaatt | |
| | | gtccttttaacagcgatcgcgtatttcgtctcgctcaggcgcaatcacgaatgaataacg | |
| | | gtttggttgatgcgagtgattttgatgacgagcgtaatggctggcctgttgaacaagtct | |
| | | ggaaagaaatgcataaacttttgccattctcaccggattcagtcgtcactcatggtgattt | |
| | | ctcacttgataaccttatttttgacgaggggaaattaataggttgtattgatgttggacga | |
| | | gtcggaatcgcagaccgataccaggatcttgccatcctatggaactgcctcggtgagt | |
| | | tttctccttcattacagaaacggctttttcaaaaatatggtattgataatcctgatatgaata | |
| | | aattgcagtttcatttgatgctcgatgagtttttctaatctcatgaccaaaatcccttaacgt | |
| | | gagttttcgttccactgagcgtcagaccccgtagaaaagatcaaaggatcttcttgaga | |
| | | tcctttttttctgcgcgtaatctgctgcttgcaaacaaaaaaaccaccgctaccagcggt | |
| | | ggtttgtttgccggatcaagagctaccaactctttttccgaaggtaactggcttcagcag | |
| | | agcgcagataccaaatactgtccttctagtgtagccgtagttaggccaccacttcaaga | |
| | | actctgtagcaccgcctacatacctcgctctgctaatcctgttaccagtggctgctgcca | |
| | | gtggcgataagtcgtgtcttaccgggttggactcaagacgatagttaccggataaggc | |
| | | gcagcggtcgggctgaacggggggttcgtgcacacagcccagcttggagcgaacg | |
| | | acctacaccgaactgagatacctacagcgtgagctatgagaaagcgccacgcttccc | |
| | | gaagggagaaaggcggacaggtatccggtaagcggcagggtcggaacaggagag | |
| | | cgcacgagggagcttccagggggaaacgcctggtatctttatagtcctgtcgggtttc | |
| | | gccacctctgacttgagcgtcgatttttgtgatgctcgtcaggggggcggagcctatgg | |
| | | aaaaacgccagcaacgcggcctttttacggttcctggccttttgctggccttttgctcac | |
| | | atgtgcggccgcacgcgtcatatttatggggtatatgtgaatatttattaca | |
| Anti-CD30 | Coding sequence | cagatccagctgcagcagagcggccccgaggtggtgaagcccggcgccagcgtg | 119 |
| CAR 4- | | aagatcagctgcaaggccagcggctacaccttcaccgactactacatcacctgggtg | |
| P2A-HLA- | | aagcagaagcccggccagggcctggagtggatcggctggatctaccccggcagcg | |
| E trimer | | gcaacaccaagtacaacgagaagttcaagggcaaggccaccctgaccgtggacac | |
| | | cagcagcagcaccgccttcatgcagctgagcagcctgaccagcgaggacaccgcc | |
| | | gtgtacttctgcgccaactacggcaactactggttcgcctactggggccagggcaccc | |
| | | aggtgaccgtgagcgccggcggcggcggcagcggcggcggcggcagcggcgg | |
| | | cggcggcagcgacatcgtgctgacccagagccccgccagcctggccgtgagcctg | |
| | | ggccagagagccaccatcagctgcaaggccagccagagcgtggacttcgacggcg | |
| | | acagctacatgaactggtaccagcagaagcccggccagccccccaaggtgctgatc | |
| | | tacgccgccagcaacctggagagcggcatccccgccagattcagcggcagcggca | |
| | | gcggcaccgacttcaccctgaacatccaccccgtggaggaggaggacgccgccac | |
| | | ctactactgccagcagagcaacgaggacccctggaccttcggcggcggcaccaag | |
| | | ctggagatcaagagcgccgccgccttcgtgcccgtgttcctgcccgccaagcccacc | |
| | | accacccccgcccccagaccccccacccccgcccccaccatcgccagccagcccc | |
| | | tgagcctgagacccgaggcctgcagacccgccgccggcggcgccgtgcacacca | |
| | | gaggcctggacttcgcctgcgacatctacatctgggcccccctggccggcacctgcg | |
| | | gcgtgctgctgctgagcctggtgatcaccctgtactgcaaccacagaaacagaagca | |
| | | agagaagcagactgctgcacagcgactacatgaacatgacccccagaagacccgg | |
| | | ccccaccagaaagcactaccagccctacgccccccccagagacttcgccgcctaca | |
| | | gaagcagagtgaagttcagcagaagcgccgacgcccccgcctaccagcagggcca | |
| | | gaaccagctgtacaacgagctgaacctgggcagaagagaggagtacgacgtgctg | |
| | | gacaagagaagaggcagagaccccgagatgggcggcaagcccagaagaaagaa | |
| | | cccccaggagggcctgtacaacgagctgcagaaggacaagatggccgaggcctac | |
| | | agcgagatcggcatgaagggcgagagaagaagaggcaagggccacgacggcct | |
| | | gtaccagggcctgagcaccgccaccaaggacacctacgacgccctgcacatgcag | |
| | | gccctgccccccagaggaagcggagctactaacttcagcctgctgaagcaggctgg | |
| | | agacgtggaggagaaccctggacctatgtctcgctccgttgccttagctgtgctcgcg | |
| | | ctactctctctttctggattagaggctgtcatggcgccccgaaccctcttcctgggtgga | |
| | | ggcggttcaggcggaggtggctctggcggtggcggatcgatccagcgtactccaaa | |
| | | gattcaggtttactcacgtcatccagcagagaatggaaagtcaaatttcctgaattgcta | |
| | | tgtgtctgggtttcatccatccgacattgaagttgacttactgaagaatggagagagaat | |
| | | tgaaaaagtggagcattcagacttgtctttcagcaaggactggtctttctatctcttgtact | |
| | | acactgaattcacccccactgaaaaagatgagtatgcctgccgtgtgaaccatgtgact | |
| | | ttgtcacagcccaagatagttaagtgggatcgagacatgggtggtggtggttctggtg | |
| | | gtggtggttctggcggcggcggctccggtggtggtggatccggctcccactccttga | |
| | | agtatttccacacttccgtgtcccggcccggccgcggggagccccgcttcatctctgt | |
| | | gggctacgtggacgacacccagttcgtgcgcttcgacaacgacgccgcgagtccga | |
| | | ggatggtgccgcgggcgccgtggatggagcaggaggggtcagagtattgggaccg | |
| | | ggagacacggagcgccagggacaccgcacagattttccgagtgaatctgcggacg | |
| | | ctgcgcggctactacaatcagagcgaggccgggtctcacaccctgcagtggatgcat | |
| | | ggctgcgagctggggcccgacgggcgcttcctccgcgggtatgaacagttcgccta | |
| | | cgacggcaaggattatctcaccctgaatgaggacctgcgctcctggaccgcggtgga | |
| | | cacggcggctcagatctccgagcaaaagtcaaatgatgcctctgaggcggagcacc | |
| | | agagagcctacctggaagacacatgcgtggagtggctccacaaatacctggagaag | |
| | | gggaaggagacgctgcttcacctggagcccccaaagacacacgtgactcaccaccc | |
| | | catctctgaccatgaggccaccctgaggtgctgggccctgggcttctaccctgcgga | |
| | | gatcacactgacctggcagcaggatggggagggccatacccaggacacggagctc | |
| | | gtggagaccaggcctgcaggggatggaaccttccagaagtgggcagctgtggtggt | |
| | | gccttctggagaggagcagagatacacgtgccatgtgcagcatgaggggctacccg | |
| | | agcccgtcaccctgagatggaagccggcttcccagcccaccatccccatcgtgggc | |
| | | atcattgctggcctggttctccttggatctgtggtctctggagctgtggttgctgctgtgat | |
| | | atggaggaagaagagctcaggtggaaaaggagggagctactctaaggctgagtgg | |
| | | agcgacagtgcccaggggtctgagtctcacagcttg | |
| Anti-CD30 | Coding sequence | caggtgcagctgcagcagtggggcgccggcctgctgaagcccagcgagaccctga | 120 |
| CAR 5- | | gcctgacctgcgccgtgtacggcggcagcttcagcgcctactactggagctggatca | |
| P2A-HLA- | | gacagccccccggcaagggcctggagtggatcggcgacatcaaccacggcggcg | |
| E trimer | | gcaccaactacaaccccagcctgaagagcagagtgaccatcagcgtggacaccag | |
| | | caagaaccagttcagcctgaagctgaacagcgtgaccgccgccgacaccgccgtgt | |
| | | actactgcgccagcctgaccgcctactggggccagggcagcctggtgaccgtgagc | |
| | | agcggcggcggcggcagcggcggcggcggcagcggcggcggcggcagcgaca | |
| | | tccagatgacccagagccccaccagcctgagcgccagcgtgggcgacagagtgac | |
| | | catcacctgcagagccagccagggcatcagcagctggctgacctggtaccagcaga | |
| | | agcccgagaaggcccccaagagcctgatctacgccgccagcagcctgcagagcgg | |
| | | cgtgcccagcagattcagcggcagcggcagcggcaccgacttcaccctgaccatca | |
| | | gcagcctgcagcccgaggacttcgccacctactactgccagcagtacgacagctacc | |
| | | ccatcaccttcggccagggcaccagactggagatcaagagcgccgccgccttcgtg | |
| | | cccgtgttcctgcccgccaagcccaccaccacccccgcccccagaccccccacccc | |
| | | cgcccccaccatcgccagccagcccctgagcctgagacccgaggcctgcagaccc | |
| | | gccgccggcggcgccgtgcacaccagaggcctggacttcgcctgcgacatctacat | |
| | | ctgggcccccctggccggcacctgcggcgtgctgctgctgagcctggtgatcaccct | |
| | | gtactgcaaccacagaaacagaaagagaggcagaaagaagctgctgtacatcttcaa | |
| | | gcagcccttcatgagacccgtgcagaccacccaggaggaggacggctgcagctgc | |
| | | agattccccgaggaggaggagggcggctgcgagctgagagtgaagttcagcagaa | |
| | | gcgccgacgcccccgcctaccagcagggccagaaccagctgtacaacgagctgaa | |
| | | cctgggcagaagagaggagtacgacgtgctggacaagagaagaggcagagaccc | |
| | | cgagatgggcggcaagcccagaagaaagaacccccaggagggcctgtacaacga | |
| | | gctgcagaaggacaagatggccgaggcctacagcgagatcggcatgaagggcga | |
| | | gagaagaagaggcaagggccacgacggcctgtaccagggcctgagcaccgccac | |
| | | caaggacacctacgacgccctgcacatgcaggccctgccccccagaggaagcgga | |
| | | ttcagcctgctgaagcaggctggagacgtggaggagaaccctggacctatgtctcgc | |
| | | tccgttgccttagctgtgctcgcgctactctctctttctggattagaggctgtcatggcgc | |
| | | cccgaaccctcttcctgggtggaggcggttcaggcggaggtggctctggcggtggc | |
| | | ggatcgatccagcgtactccaaagattcaggtttactcacgtcatccagcagagaatg | |
| | | gaaagtcaaatttcctgaattgctatgtgtctgggtttcatccatccgacattgaagttga | |
| | | cttactgaagaatggagagagaattgaaaaagtggagcattcagacttgtctttcagca | |
| | | aggactggtctttctatctcttgtactacactgaattcacccccactgaaaaagatgagta | |
| | | tgcctgccgtgtgaaccatgtgactttgtcacagcccaagatagttaagtgggatcgag | |
| | | acatgggtggtggtggttctggtggtggtggttctggcggcggcggctccggtggtg | |
| | | gtggatccggctcccactccttgaagtatttccacacttccgtgtcccggcccggccgc | |
| | | ggggagccccgcttcatctctgtgggctacgtggacgacacccagttcgtgcgcttcg | |
| | | acaacgacgccgcgagtccgaggatggtgccgcgggcgccgtggatggagcagg | |
| | | aggggtcagagtattgggaccgggagacacggagcgccagggacaccgcacaga | |
| | | ttttccgagtgaatctgcggacgctgcgcggctactacaatcagagcgaggccgggt | |
| | | ctcacaccctgcagtggatgcatggctgcgagctggggcccgacgggcgcttcctcc | |
| | | gcgggtatgaacagttcgcctacgacggcaaggattatctcaccctgaatgaggacct | |
| | | gcgctcctggaccgcggtggacacggcggctcagatctccgagcaaaagtcaaatg | |
| | | atgcctctgaggcggagcaccagagagcctacctggaagacacatgcgtggagtgg | |
| | | ctccacaaatacctggagaaggggaaggagacgctgcttcacctggagcccccaaa | |
| | | gacacacgtgactcaccaccccatctctgaccatgaggccaccctgaggtgctgggc | |
| | | cctgggcttctaccctgcggagatcacactgacctggcagcaggatggggagggcc | |
| | | atacccaggacacggagctcgtggagaccaggcctgcaggggatggaaccttcca | |
| | | gaagtgggcagctgtggtggtgccttctggagaggagcagagatacacgtgccatgt | |
| | | gcagcatgaggggctacccgagcccgtcaccctgagatggaagccggcttcccagc | |
| | | ccaccatccccatcgtgggcatcattgctggcctggttctccttggatctgtggtctctg | |
| | | gagctgtggttgctgctgtgatatggaggaagaagagctcaggtggaaaaggaggg | |
| | | agctactctaaggctgagtggagcgacagtgcccaggggtctgagtctcacagcttg | |
| Anti-CD30 | Coding sequence | gacatccagatgacccagagccccaccagcctgagcgccagcgtgggcgacagag | 121 |
| CAR 6- | | tgaccatcacctgcagagccagccagggcatcagcagctggctgacctggtaccag | |
| P2A-HLA- | | cagaagcccgagaaggcccccaagagcctgatctacgccgccagcagcctgcaga | |
| E trimer | | gcggcgtgcccagcagattcagcggcagcggcagcggcaccgacttcaccctgac | |
| | | catcagcagcctgcagcccgaggacttcgccacctactactgccagcagtacgacag | |
| | | ctaccccatcaccttcggccagggcaccagactggagatcaagggcggcggcggc | |
| | | agcggcggcggcggcagcggcggcggcggcagccaggtgcagctgcagcagtg | |
| | | gggcgccggcctgctgaagcccagcgagaccctgagcctgacctgcgccgtgtac | |
| | | ggcggcagcttcagcgcctactactggagctggatcagacagccccccggcaagg | |
| | | gcctggagtggatcggcgacatcaaccacggcggcggcaccaactacaaccccag | |
| | | cctgaagagcagagtgaccatcagcgtggacaccagcaagaaccagttcagcctga | |
| | | agctgaacagcgtgaccgccgccgacaccgccgtgtactactgcgccagcctgacc | |
| | | gcctactggggccagggcagcctggtgaccgtgagcgccgccgccttcgtgcccgt | |
| | | gttcctgcccgccaagcccaccaccacccccgcccccagaccccccacccccgcc | |
| | | cccaccatcgccagccagcccctgagcctgagacccgaggcctgcagacccgccg | |
| | | ccggcggcgccgtgcacaccagaggcctggacttcgcctgcgacatctacatctgg | |
| | | gcccccctggccggcacctgcggcgtgctgctgctgagcctggtgatcaccctgtac | |
| | | tgcaaccacagaaacagaaagagaggcagaaagaagctgctgtacatcttcaagca | |
| | | gcccttcatgagacccgtgcagaccacccaggaggaggacggctgcagctgcagat | |
| | | tccccgaggaggaggagggcggctgcgagctgagagtgaagttcagcagaagcg | |
| | | ccgacgcccccgcctaccagcagggccagaaccagctgtacaacgagctgaacct | |
| | | gggcagaagagaggagtacgacgtgctggacaagagaagaggcagagaccccga | |
| | | gatgggcggcaagcccagaagaaagaacccccaggagggcctgtacaacgagct | |
| | | gcagaaggacaagatggccgaggcctacagcgagatcggcatgaagggcgagag | |
| | | aagaagaggcaagggccacgacggcctgtaccagggcctgagcaccgccaccaa | |
| | | ggacacctacgacgccctgcacatgcaggccctgccccccagaggaagcggagct | |
| | | actaacttcagcctgctgaagcaggctggagacgtggaggagaaccctggacctatg | |
| | | tctcgctccgttgccttagctgtgctcgcgctactctctctttctggattagaggctgtcat | |
| | | ggcgccccgaaccctcttcctgggtggaggcggttcaggcggaggtggctctggcg | |
| | | gtggcggatcgatccagcgtactccaaagattcaggtttactcacgtcatccagcaga | |
| | | gaatggaaagtcaaatttcctgaattgctatgtgtctgggtttcatccatccgacattgaa | |
| | | gttgacttactgaagaatggagagagaattgaaaaagtggagcattcagacttgtctttc | |
| | | agcaaggactggtctttctatctcttgtactacactgaattcacccccactgaaaaagat | |
| | | gagtatgcctgccgtgtgaaccatgtgactttgtcacagcccaagatagttaagtggga | |
| | | tcgagacatgggtggtggtggttctggtggtggtggttctggcggcggcggctccgg | |
| | | tggtggtggatccggctcccactccttgaagtatttccacacttccgtgtcccggcccg | |
| | | gccgcggggagccccgcttcatctctgtgggctacgtggacgacacccagttcgtgc | |
| | | gcttcgacaacgacgccgcgagtccgaggatggtgccgcgggcgccgtggatgga | |
| | | gcaggaggggtcagagtattgggaccgggagacacggagcgccagggacaccgc | |
| | | acagattttccgagtgaatctgcggacgctgcgcggctactacaatcagagcgaggc | |
| | | cgggtctcacaccctgcagtggatgcatggctgcgagctggggcccgacgggcgct | |
| | | tcctccgcgggtatgaacagttcgcctacgacggcaaggattatctcaccctgaatga | |
| | | ggacctgcgctcctggaccgcggtggacacggcggctcagatctccgagcaaaagt | |
| | | caaatgatgcctctgaggcggagcaccagagagcctacctggaagacacatgcgtg | |
| | | gagtggctccacaaatacctggagaaggggaaggagacgctgcttcacctggagcc | |
| | | cccaaagacacacgtgactcaccaccccatctctgaccatgaggccaccctgaggtg | |
| | | ctgggccctgggcttctaccctgcggagatcacactgacctggcagcaggatgggg | |
| | | agggccatacccaggacacggagctcgtggagaccaggcctgcaggggatggaa | |
| | | ccttccagaagtgggcagctgtggtggtgccttctggagaggagcagagatacacgt | |
| | | gccatgtgcagcatgaggggctacccgagcccgtcaccctgagatggaagccggct | |
| | | tcccagcccaccatccccatcgtgggcatcattgctggcctggttctccttggatctgtg | |
| | | gtctctggagctgtggttgctgctgtgatatggaggaagaagagctcaggtggaaaag | |
| | | gagggagctactctaaggctgagtggagcgacagtgcccaggggtctgagtctcac | |
| | | agcttg | |
| CD8 TM | Amino acid | SAAAFVPVFLPAKPTTTPAPRPPTPAPTIASQPLSLRPE | 122 |
| | sequence | ACRPAAGGAVHTRGLDFACDIYIWAPLAGTCGVLLLS | |
| | | LVITLYCNHRNR | |
| CD28 | Amino acid | SKRSRLLHSDYMNMTPRRPGPTRKHYQPYAPPRDFAA | 123 |
| | sequence | YRS | |
| 41BB | Amino acid | KRGRKKLLYIFKQPFMRPVQTTQEEDGCSCRFPEEEEG | 124 |
| | sequence | GCEL | |
| CD3-zeta | Amino acid | RVKFSRSADAPAYQQGQNQLYNELNLGRREEYDVLD | 125 |
| | sequence | KRRGRDPEMGGKPRRKNPQEGLYNELQKDKMAEAY | |
| | | SEIGMKGERRRGKGHDGLYQGLSTATKDTYDALHMQ | |
| | | ALPPR | |
| CD8 TM | Coding sequence- | agcgccgccgccttcgtgcccgtgttcctgcccgccaagcccaccaccacccccgc | 126 |
| | codon | ccccagaccccccacccccgcccccaccatcgccagccagcccctgagcctgaga | |
| | optimized | cccgaggcctgcagacccgccgccggcggcgccgtgcacaccagaggcctggac | |
| | | ttcgcctgcgacatctacatctgggcccccctggccggcacctgcggcgtgctgctgc | |
| | | tgagcctggtgatcaccctgtactgcaaccacagaaacaga | |
| 41BB | Coding sequence- | aagagaggcagaaagaagctgctgtacatcttcaagcagcccttcatgagacccgtg | 127 |
| | codon | cagaccacccaggaggaggacggctgcagctgcagattccccgaggaggaggag | |
| | optimized | ggcggctgcgagctg | |
| CD3-zeta | Coding sequence- | agagtgaagttcagcagaagcgccgacgcccccgcctaccagcagggccagaacc | 128 |
| | codon | agctgtacaacgagctgaacctgggcagaagagaggagtacgacgtgctggacaa | |
| | optimized | gagaagaggcagagaccccgagatgggcggcaagcccagaagaaagaaccccc | |
| | | aggagggcctgtacaacgagctgcagaaggacaagatggccgaggcctacagcga | |
| | | gatcggcatgaagggcgagagaagaagaggcaagggccacgacggcctgtacca | |
| | | gggcctgagcaccgccaccaaggacacctacgacgccctgcacatgcaggccctg | |
| | | ccccccaga | |
| SERPIN | Coding sequence | atggaaactctttctaatgcaagtggtacttttgccatacgccttttaaagatactgtgtca | 129 |
| B9 CDS | | agataacccttcgcacaacgtgttctgttctcctgtgagcatctcctctgccctggccat | |
| | | ggttctcctaggggcaaagggaaacaccgcaacccagatggcccaggcactgtcttt | |
| | | aaacacagaggaagacattcatcgggctttccagtcgcttctcactgaagtgaacaag | |
| | | gctggcacacagtacctgctgagaacggccaacaggctctttggagagaaaacttgt | |
| | | cagttcctctcaacgtttaaggaatcctgtcttcaattctaccatgctgagctgaaggag | |
| | | ctttcctttatcagagctgcagaagagtccaggaaacacatcaacacctgggtctcaaa | |
| | | aaagaccgaaggtaaaattgaagagttgttgccgggtagctcaattgatgcagaaacc | |
| | | aggctggttcttgtcaatgccatctacttcaaaggaaagtggaatgaaccgtttgacga | |
| | | aacatacacaagggaaatgccctttaaaataaaccaggaggagcaaaggccagtgc | |
| | | agatgatgtatcaggaggccacgtttaagctcgcccacgtgggcgaggtgcgcgcg | |
| | | cagctgctggagctgccctacgccaggaaggagctgagcctgctggtgctgctgcct | |
| | | gacgacggcgtggagctcagcacggtggaaaaaagtctcacttttgagaaactcaca | |
| | | gcctggaccaagccagactgtatgaagagtactgaggttgaagttctccttccaaaatt | |
| | | taaactacaagaggattatgacatggaatctgtgcttcggcatttgggaattgttgatgc | |
| | | cttccaacagggcaaggctgacttgtcggcaatgtcagcggagagagacctgtgtct | |
| | | gtccaagttcgtgcacaagagttttgtggaggtgaatgaagaaggcaccgaggcagc | |
| | | ggcagcgtcgagctgctttgtagttgcagagtgctgcatggaatctggccccaggttct | |
| | | gtgctgaccaccctttccttttcttcatcaggcacaacagagccaacagcattctgttctg | |
| | | tggcaggttctcatcgcca | |
| (B2M) | | cctgcaggcagctgcgcgctcgctcgctcactgaggccgcccgggcgtcgggcga | 130 |
| SERPIN | | cctttggtcgcccggcctcagtgagcgagcgagcgcgcagagagggagtggccaa | |
| B9-P2A- | | ctccatcactaggggttcctgcggccgcacgcgtgttctagggtggaaactaagaga | |
| HLA-E | | atgatgtacctagagggcgctggaagctctaaagccctagcagttactgcttttactatt | |
| donor | | agtggtcgtttttttctcccccccgccccccgacaaatcaacagaacaaagaaaattac | |
| plasmid | | ctaaacagcaaggacatagggaggaacttcttggcacagaactttccaaacactttttc | |
| | | ctgaagggatacaagaagcaagaaaggtactctttcactaggaccttctctgagctgtc | |
| | | ctcaggatgcttttgggactatttttcttacccagagaatggagaaaccctgcagggaat | |
| | | tcccaagctgtagttataaacagaagttctccttctgctaggtagcattcaaagatcttaa | |
| | | tcttctgggtttccgttttctcgaatgaaaaatgcaggtccgagcagttaactggctggg | |
| | | gcaccattagcaagtcacttagcatctctggggccagtctgcaaagcgagggggcag | |
| | | ccttaatgtgcctccagcctgaagtcctagaatgagcgcccggtgtcccaagctgggg | |
| | | cgcgcaccccagatcggagggcgccgatgtacagacagcaaactcacccagtctag | |
| | | tgcatgccttcttaaacatcacgagactctaagaaaaggaaactgaaaacgggaaagt | |
| | | ccctctctctaacctggcactgcgtcgctggcttggagacaggtgacggtccctgcgg | |
| | | gccttgtcctgattggctgggcacgcgtttaatataagtggaggcgtcgcgctggcgg | |
| | | gcattcctgaagctaagcttgtggacgatatcgaattcgcacgacattgattattgacta | |
| | | gttattaatagtaatcaattacggggtcattagttcatagcccatatatggagttccgcgtt | |
| | | acataacttacggtaaatggcccgcctggctgaccgcccaacgacccccgcccattg | |
| | | acgtcaataatgacgtatgttcccatagtaacgccaatagggactttccattgacgtcaa | |
| | | tgggtggactatttacggtaaactgcccacttggcagtacatcaagtgtatcatatgcca | |
| | | agtacgccccctattgacgtcaatgacggtaaatggcccgcctggcattatgcccagt | |
| | | acatgaccttatgggactttcctacttggcagtacatctacgtattagtcatcgctattacc | |
| | | atgggtcgaggtgagccccacgttctgcttcactctccccatctcccccccctccccac | |
| | | ccccaattttgtatttatttattttttaattattttgtgcagcgatgggggcgggggggggg | |
| | | ggggcgcgcgccaggcggggcggggcggggcgaggggcggggcggggcgag | |
| | | gcggagaggtgcggcggcagccaatcagagcggcgcgctccgaaagtttccttttat | |
| | | ggcgaggcggcggcggcggcggccctataaaaagcgaagcgcgcggcgggcgg | |
| | | gagtcgctgcgttgccttcgccccgtgccccgctccgcgccgcctcgcgccgcccg | |
| | | ccccggctctgactgaccgcgttactcccacaggtgagcgggcgggacggcccttct | |
| | | cctccgggctgtaattagcgcttggtttaatgacggctcgtttcttttctgtggctgcgtga | |
| | | aagccttaaagggctccgggagggccctttgtgcgggggggagcggctcgggggg | |
| | | tgcgtgcgtgtgtgtgtgcgtggggagcgccgcgtgcggcccgcgctgcccggcg | |
| | | gctgtgagcgctgcgggcgcggcgcggggctttgtgcgctccgcgtgtgcgcgag | |
| | | gggagcgcggccgggggcggtgccccgcggtgcgggggggctgcgaggggaa | |
| | | caaaggctgcgtgcggggtgtgtgcgtgggggggtgagcagggggtgtgggcgc | |
| | | ggcggtcgggctgtaacccccccctgcacccccctccccgagttgctgagcacggc | |
| | | ccggcttcgggtgcggggctccgtgcggggcgtggcgcggggctcgccgtgccgg | |
| | | gcggggggtggcggcaggtgggggtgccgggcggggcggggccgcctcgggc | |
| | | cggggagggctcgggggaggggcgcggcggccccggagcgccggcggctgtc | |
| | | gaggcgcggcgagccgcagccattgccttttatggtaatcgtgcgagagggcgcag | |
| | | ggacttcctttgtcccaaatctggcggagccgaaatctgggaggcgccgccgcaccc | |
| | | cctctagcgggcgcgggcgaagcggtgcggcgccggcaggaaggaaatgggcg | |
| | | gggagggccttcgtgcgtcgccgcgccgccgtccccttctccatctccagcctcggg | |
| | | gctgccgcagggggacggctgccttcgggggggacggggcagggcggggttcgg | |
| | | cttctggcgtgtgaccggcggctctagagcctctgctaaccatgttcatgccttcttctttt | |
| | | tcctacaggggggatccgtttatctgcagaattcgcccttgacgtcgccaccatggaaa | |
| | | ctctttctaatgcaagtggtacttttgccatacgccttttaaagatactgtgtcaagataac | |
| | | ccttcgcacaacgtgttctgttctcctgtgagcatctcctctgccctggccatggttctcct | |
| | | aggggcaaagggaaacaccgcaacccagatggcccaggcactgtctttaaacacag | |
| | | aggaagacattcatcgggctttccagtcgcttctcactgaagtgaacaaggctggcac | |
| | | acagtacctgctgagaacggccaacaggctctttggagagaaaacttgtcagttcctct | |
| | | caacgtttaaggaatcctgtcttcaattctaccatgctgagctgaaggagctttcctttatc | |
| | | agagctgcagaagagtccaggaaacacatcaacacctgggtctcaaaaaagaccga | |
| | | aggtaaaattgaagagttgttgccgggtagctcaattgatgcagaaaccaggctggttc | |
| | | ttgtcaatgccatctacttcaaaggaaagtggaatgaaccgtttgacgaaacatacaca | |
| | | agggaaatgccctttaaaataaaccaggaggagcaaaggccagtgcagatgatgtat | |
| | | caggaggccacgtttaagctcgcccacgtgggcgaggtgcgcgcgcagctgctgg | |
| | | agctgccctacgccaggaaggagctgagcctgctggtgctgctgcctgacgacggc | |
| | | gtggagctcagcacggtggaaaaaagtctcacttttgagaaactcacagcctggacc | |
| | | aagccagactgtatgaagagtactgaggttgaagttctccttccaaaatttaaactacaa | |
| | | gaggattatgacatggaatctgtgcttcggcatttgggaattgttgatgccttccaacag | |
| | | ggcaaggctgacttgtcggcaatgtcagcggagagagacctgtgtctgtccaagttcg | |
| | | tgcacaagagttttgtggaggtgaatgaagaaggcaccgaggcagcggcagcgtcg | |
| | | agctgctttgtagttgcagagtgctgcatggaatctggccccaggttctgtgctgacca | |
| | | ccctttccttttcttcatcaggcacaacagagccaacagcattctgttctgtggcaggttc | |
| | | tcatcgccaggaagcggagctactaacttcagcctgctgaagcaggctggagacgtg | |
| | | gaggagaaccctggacctatgtctcgctccgttgccttagctgtgctcgcgctactctct | |
| | | ctttctggattagaggctgtcatggcgccccgaaccctcttcctgggtggaggcggttc | |
| | | aggcggaggtggctctggcggtggcggatcgatccagcgtactccaaagattcaggt | |
| | | ttactcacgtcatccagcagagaatggaaagtcaaatttcctgaattgctatgtgtctgg | |
| | | gtttcatccatccgacattgaagttgacttactgaagaatggagagagaattgaaaaag | |
| | | tggagcattcagacttgtctttcagcaaggactggtctttctatctcttgtactacactgaa | |
| | | ttcacccccactgaaaaagatgagtatgcctgccgtgtgaaccatgtgactttgtcaca | |
| | | gcccaagatagttaagtgggatcgagacatgggtggtggtggttctggtggtggtggt | |
| | | tctggcggcggcggctccggtggtggtggatccggctcccactccttgaagtatttcc | |
| | | acacttccgtgtcccggcccggccgcggggagccccgcttcatctctgtgggctacg | |
| | | tggacgacacccagttcgtgcgcttcgacaacgacgccgcgagtccgaggatggtg | |
| | | ccgcgggcgccgtggatggagcaggaggggtcagagtattgggaccgggagaca | |
| | | cggagcgccagggacaccgcacagattttccgagtgaatctgcggacgctgcgcgg | |
| | | ctactacaatcagagcgaggccgggtctcacaccctgcagtggatgcatggctgcga | |
| | | gctggggcccgacgggcgcttcctccgcgggtatgaacagttcgcctacgacggca | |
| | | aggattatctcaccctgaatgaggacctgcgctcctggaccgcggtggacacggcgg | |
| | | ctcagatctccgagcaaaagtcaaatgatgcctctgaggcggagcaccagagagcct | |
| | | acctggaagacacatgcgtggagtggctccacaaatacctggagaaggggaagga | |
| | | gacgctgcttcacctggagcccccaaagacacacgtgactcaccaccccatctctga | |
| | | ccatgaggccaccctgaggtgctgggccctgggcttctaccctgcggagatcacact | |
| | | gacctggcagcaggatggggagggccatacccaggacacggagctcgtggagac | |
| | | caggcctgcaggggatggaaccttccagaagtgggcagctgtggtggtgccttctgg | |
| | | agaggagcagagatacacgtgccatgtgcagcatgaggggctacccgagcccgtc | |
| | | accctgagatggaagccggcttcccagcccaccatccccatcgtgggcatcattgctg | |
| | | gcctggttctccttggatctgtggtctctggagctgtggttgctgctgtgatatggagga | |
| | | agaagagctcaggtggaaaaggagggagctactctaaggctgagtggagcgacag | |
| | | tgcccaggggtctgagtctcacagcttgtaatgatagccgctgatcagcctcgactgtg | |
| | | ccttctagttgccagccatctgttgtttgcccctcccccgtgccttccttgaccctggaag | |
| | | gtgccactcccactgtcctttcctaataaaatgaggaaattgcatcgcattgtctgagta | |
| | | ggtgtcattctattctggggggtggggtggggcaggacagcaagggggaggattgg | |
| | | gaagacaatagcaggcatgctggggatgcggtgggctctatgggtcgacccagcgt | |
| | | gagtctctcctaccctcccgctctggtccttcctctcccgctctgcaccctctgtggccct | |
| | | cgctgtgctctctcgctccgtgacttcccttctccaagttctccttggtggcccgccgtg | |
| | | gggctagtccagggctggatctcggggaagcggcggggtggcctgggagtgggg | |
| | | aagggggtgcgcacccgggacgcgcgctacttgcccctttcggcggggagcaggg | |
| | | gagacctttggcctacggcgacgggagggtcgggacaaagtttagggcgtcgataa | |
| | | gcgtcagagcgccgaggttgggggagggtttctcttccgctctttcgcggggcctctg | |
| | | gctcccccagcgcagctggagtgggggacgggtaggctcgtcccaaaggcgcggc | |
| | | gctgaggtttgtgaacgcgtggaggggcgcttggggtctgggggaggcgtcgcccg | |
| | | ggtaagcctgtctgctgcggctctgcttcccttagactggagagctgtggacttcgtcta | |
| | | ggcgcccgctaagttcgcatgtcctagcacctctgggtctatgtggggccacaccgtg | |
| | | gggaggaaacagcacgcgacgtttgtagaatgcttggctgtgatacaaagcggtttcg | |
| | | aataattaacttatttgttcccatcacatgtcacttttaaaaaattataagaactacccgttat | |
| | | tgacatctttctgtgtgccaaggactttatgtgctttgcgtcatttaattttgaaaacagttat | |
| | | cttccgccatagataactactatggttatcttctggtaaccacgtgcggaccgaggctg | |
| | | cagcgtcgtcctccctaggaacccctagtgatggagttggccactccctctctgcgcg | |
| | | ctcgctcgctcactgaggccgggcgaccaaaggtcgcccgacgcccgggctttgcc | |
| | | cgggcggcctcagtgagcgagcgagcgcgcagctgcctgcaggggcgcctgatg | |
| | | cggtattttctccttacgcatctgtgcggtatttcacaccgcatacgtcaaagcaaccata | |
| | | gtacgcgccctgtagcggcgcattaagcgcggcgggtgtggtggttacgcgcagcg | |
| | | tgaccgctacacttgccagcgccctagcgcccgctcctttcgctttcttcccttcctttctc | |
| | | gccacgttcgccggctttccccgtcaagctctaaatcgggggctccctttagggttccg | |
| | | atttagtgctttacggcacctcgaccccaaaaaacttgatttgggtgatggttcacgtagt | |
| | | gggccatcgccctgatagacggtttttcgccctttgacgttggagtccacgttctttaata | |
| | | gtggactcttgttccaaactggaacaacactcaaccctatctcgggctattcttttgattta | |
| | | taagggattttgccgatttcggcctattggttaaaaaatgagctgatttaacaaaaatttaa | |
| | | cgcgaattttaacaaaatattaacgtttacaattttatggtgcactctcagtacaatctgct | |
| | | ctgatgccgcatagttaagccagccccgacacccgccaacacccgctgacgcgccc | |
| | | tgacgggcttgtctgctcccggcatccgcttacagacaagctgtgaccgtctccggga | |
| | | gctgcatgtgtcagaggttttcaccgtcatcaccgaaacgcgcgagacgaaagggcc | |
| | | tcgtgatacgcctatttttataggttaatgtcatgaacaataaaactgtctgcttacataaa | |
| | | cagtaatacaaggggtgttatgagccatattcaacgggaaacgtcgaggccgcgatta | |
| | | aattccaacatggatgctgatttatatgggtataaatgggctcgcgataatgtcgggcaa | |
| | | tcaggtgcgacaatctatcgcttgtatgggaagcccgatgcgccagagttgtttctgaa | |
| | | acatggcaaaggtagcgttgccaatgatgttacagatgagatggtcagactaaactgg | |
| | | ctgacggaatttatgcctcttccgaccatcaagcattttatccgtactcctgatgatgcat | |
| | | ggttactcaccactgcgatccccggaaaaacagcattccaggtattagaagaatatcct | |
| | | gattcaggtgaaaatattgttgatgcgctggcagtgttcctgcgccggttgcattcgatt | |
| | | cctgtttgtaattgtccttttaacagcgatcgcgtatttcgtctcgctcaggcgcaatcac | |
| | | gaatgaataacggtttggttgatgcgagtgattttgatgacgagcgtaatggctggcct | |
| | | gttgaacaagtctggaaagaaatgcataaacttttgccattctcaccggattcagtcgtc | |
| | | actcatggtgatttctcacttgataaccttatttttgacgaggggaaattaataggttgtatt | |
| | | gatgttggacgagtcggaatcgcagaccgataccaggatcttgccatcctatggaact | |
| | | gcctcggtgagttttctccttcattacagaaacggctttttcaaaaatatggtattgataat | |
| | | cctgatatgaataaattgcagtttcatttgatgctcgatgagtttttctaatctcatgaccaa | |
| | | aatcccttaacgtgagttttcgttccactgagcgtcagaccccgtagaaaagatcaaag | |
| | | gatcttcttgagatcctttttttctgcgcgtaatctgctgcttgcaaacaaaaaaaccacc | |
| | | gctaccagcggtggtttgtttgccggatcaagagctaccaactctttttccgaaggtaac | |
| | | tggcttcagcagagcgcagataccaaatactgtccttctagtgtagccgtagttaggcc | |
| | | accacttcaagaactctgtagcaccgcctacatacctcgctctgctaatcctgttaccag | |
| | | tggctgctgccagtggcgataagtcgtgtcttaccgggttggactcaagacgatagtta | |
| | | ccggataaggcgcagcggtcgggctgaacggggggttcgtgcacacagcccagct | |
| | | tggagcgaacgacctacaccgaactgagatacctacagcgtgagctatgagaaagc | |
| | | gccacgcttcccgaagggagaaaggcggacaggtatccggtaagcggcagggtcg | |
| | | gaacaggagagcgcacgagggagcttccagggggaaacgcctggtatctttatagt | |
| | | cctgtcgggtttcgccacctctgacttgagcgtcgatttttgtgatgctcgtcagggggg | |
| | | cggagcctatggaaaaacgccagcaacgcggcctttttacggttcctggccttttgctg | |
| | | gccttttgctcacatgt | |
| SERPIN | | atggaaactctttctaatgcaagtggtacttttgccatacgccttttaaagatactgtgtca | 131 |
| B9-P2A- | | agataacccttcgcacaacgtgttctgttctcctgtgagcatctcctctgccctggccat | |
| HLA-E | | ggttctcctaggggcaaagggaaacaccgcaacccagatggcccaggcactgtcttt | |
| transgene | | aaacacagaggaagacattcatcgggctttccagtcgcttctcactgaagtgaacaag | |
| | | gctggcacacagtacctgctgagaacggccaacaggctctttggagagaaaacttgt | |
| | | cagttcctctcaacgtttaaggaatcctgtcttcaattctaccatgctgagctgaaggag | |
| | | ctttcctttatcagagctgcagaagagtccaggaaacacatcaacacctgggtctcaaa | |
| | | aaagaccgaaggtaaaattgaagagttgttgccgggtagctcaattgatgcagaaacc | |
| | | aggctggttcttgtcaatgccatctacttcaaaggaaagtggaatgaaccgtttgacga | |
| | | aacatacacaagggaaatgccctttaaaataaaccaggaggagcaaaggccagtgc | |
| | | agatgatgtatcaggaggccacgtttaagctcgcccacgtgggcgaggtgcgcgcg | |
| | | cagctgctggagctgccctacgccaggaaggagctgagcctgctggtgctgctgcct | |
| | | gacgacggcgtggagctcagcacggtggaaaaaagtctcacttttgagaaactcaca | |
| | | gcctggaccaagccagactgtatgaagagtactgaggttgaagttctccttccaaaatt | |
| | | taaactacaagaggattatgacatggaatctgtgcttcggcatttgggaattgttgatgc | |
| | | cttccaacagggcaaggctgacttgtcggcaatgtcagcggagagagacctgtgtct | |
| | | gtccaagttcgtgcacaagagttttgtggaggtgaatgaagaaggcaccgaggcagc | |
| | | ggcagcgtcgagctgctttgtagttgcagagtgctgcatggaatctggccccaggttct | |
| | | gtgctgaccaccctttccttttcttcatcaggcacaacagagccaacagcattctgttctg | |
| | | tggcaggttctcatcgccaggaagcggagctactaacttcagcctgctgaagcaggct | |
| | | ggagacgtggaggagaaccctggacctatgtctcgctccgttgccttagctgtgctcg | |
| | | cgctactctctctttctggattagaggctgtcatggcgccccgaaccctcttcctgggtg | |
| | | gaggcggttcaggcggaggtggctctggcggtggcggatcgatccagcgtactcca | |
| | | aagattcaggtttactcacgtcatccagcagagaatggaaagtcaaatttcctgaattgc | |
| | | tatgtgtctgggtttcatccatccgacattgaagttgacttactgaagaatggagagaga | |
| | | attgaaaaagtggagcattcagacttgtctttcagcaaggactggtctttctatctcttgta | |
| | | ctacactgaattcacccccactgaaaaagatgagtatgcctgccgtgtgaaccatgtga | |
| | | ctttgtcacagcccaagatagttaagtgggatcgagacatgggtggtggtggttctggt | |
| | | ggtggtggttctggcggcggcggctccggtggtggtggatccggctcccactccttg | |
| | | aagtatttccacacttccgtgtcccggcccggccgcggggagccccgcttcatctctgt | |
| | | gggctacgtggacgacacccagttcgtgcgcttcgacaacgacgccgcgagtccga | |
| | | ggatggtgccgcgggcgccgtggatggagcaggaggggtcagagtattgggaccg | |
| | | ggagacacggagcgccagggacaccgcacagattttccgagtgaatctgcggacg | |
| | | ctgcgcggctactacaatcagagcgaggccgggtctcacaccctgcagtggatgcat | |
| | | ggctgcgagctggggcccgacgggcgcttcctccgcgggtatgaacagttcgccta | |
| | | cgacggcaaggattatctcaccctgaatgaggacctgcgctcctggaccgcggtgga | |
| | | cacggcggctcagatctccgagcaaaagtcaaatgatgcctctgaggcggagcacc | |
| | | agagagcctacctggaagacacatgcgtggagtggctccacaaatacctggagaag | |
| | | gggaaggagacgctgcttcacctggagcccccaaagacacacgtgactcaccaccc | |
| | | catctctgaccatgaggccaccctgaggtgctgggccctgggcttctaccctgcgga | |
| | | gatcacactgacctggcagcaggatggggagggccatacccaggacacggagctc | |
| | | gtggagaccaggcctgcaggggatggaaccttccagaagtgggcagctgtggtggt | |
| | | gccttctggagaggagcagagatacacgtgccatgtgcagcatgaggggctacccg | |
| | | agcccgtcaccctgagatggaagccggcttcccagcccaccatccccatcgtgggc | |
| | | atcattgctggcctggttctccttggatctgtggtctctggagctgtggttgctgctgtgat | |
| | | atggaggaagaagagctcaggtggaaaaggagggagctactctaaggctgagtgg | |
| | | agcgacagtgcccaggggtctgagtctcacagcttgtaatgatagccgctgatcagcc | |
| | | tcgactgtgccttctagttgccagccatctgttgtttgcccctcccccgtgccttccttgac | |
| | | cctggaaggtgccactcccactgtcctttcctaataaaa5501tgaggaaattgcatcg | |
| | | cattgtcgagtaggtgtcattctattctggggggtggggtggggcaggacagcaagg | |
| | | gggaggattgggaagacaatagcaggcatgctggggatgcggtgggctctatgg | |
| Plasmid 1 | Forward | CCCTTAACGTGAGTTTTCGTTCCACTGAGCGTCAGA | 132 |
| primer | | CCCCGTAGAAAAGATCAAAGG | |
| Plasmid 1 | Reverse | GTCCAACCCGGTAAGACACGACTTATCGCCACTGGC | 133 |
| primer | | AGCAGCCACTGGTAACAG | |
| Plasmid 2 | Forward | CACTTGCCAGCGCCCTAGCGCCCGCTCCTTTCGCTTT | 134 |
| primer | | CTTCCCTTCCTTTCTC | |
| Plasmid 2 | Reverse | GGGCGCGTCAGCGGGTGTTGGCGGGTGTCGGGG | 135 |
| primer | | | |
| (B2M) | | cctgcaggcagctgcgcgctcgctcgctcactgaggccgcccgggcgtcgggcga | 136 |
| SERPIN | | cctttggtcgcccggcctcagtgagcgagcgagcgcgcagagagggagtggccaa | |
| B9-P2A- | | ctccatcactaggggttcctgcggccgcacgcgtgttctagggtggaaactaagaga | |
| IL15/IL15 | | atgatgtacctagagggcgctggaagctctaaagccctagcagttactgcttttactatt | |
| Rα donor | | agtggtcgtttttttctcccccccgccccccgacaaatcaacagaacaaagaaaattac | |
| plasmid | | ctaaacagcaaggacatagggaggaacttcttggcacagaactttccaaacactttttc | |
| | | ctgaagggatacaagaagcaagaaaggtactctttcactaggaccttctctgagctgtc | |
| | | ctcaggatgcttttgggactatttttcttacccagagaatggagaaaccctgcagggaat | |
| | | tcccaagctgtagttataaacagaagttctccttctgctaggtagcattcaaagatcttaa | |
| | | tcttctgggtttccgttttctcgaatgaaaaatgcaggtccgagcagttaactggctggg | |
| | | gcaccattagcaagtcacttagcatctctggggccagtctgcaaagcgagggggcag | |
| | | ccttaatgtgcctccagcctgaagtcctagaatgagcgcccggtgtcccaagctgggg | |
| | | cgcgcaccccagatcggagggcgccgatgtacagacagcaaactcacccagtctag | |
| | | tgcatgccttcttaaacatcacgagactctaagaaaaggaaactgaaaacgggaaagt | |
| | | ccctctctctaacctggcactgcgtcgctggcttggagacaggtgacggtccctgcgg | |
| | | gccttgtcctgattggctgggcacgcgtttaatataagtggaggcgtcgcgctggcgg | |
| | | gcattcctgaagctaagcttgtggacgatatcgaattcgcacgacattgattattgacta | |
| | | gttattaatagtaatcaattacggggtcattagttcatagcccatatatggagttccgcgtt | |
| | | acataacttacggtaaatggcccgcctggctgaccgcccaacgacccccgcccattg | |
| | | acgtcaataatgacgtatgttcccatagtaacgccaatagggactttccattgacgtcaa | |
| | | tgggtggactatttacggtaaactgcccacttggcagtacatcaagtgtatcatatgcca | |
| | | agtacgccccctattgacgtcaatgacggtaaatggcccgcctggcattatgcccagt | |
| | | acatgaccttatgggactttcctacttggcagtacatctacgtattagtcatcgctattacc | |
| | | atgggtcgaggtgagccccacgttctgcttcactctccccatctcccccccctccccac | |
| | | ccccaattttgtatttatttattttttaattattttgtgcagcgatgggggcgggggggggg | |
| | | ggggcgcgcgccaggcggggcggggcggggcgaggggcggggcggggcgag | |
| | | gcggagaggtgcggcggcagccaatcagagcggcgcgctccgaaagtttccttttat | |
| | | ggcgaggcggcggcggcggcggccctataaaaagcgaagcgcgcggcgggcgg | |
| | | gagtcgctgcgttgccttcgccccgtgccccgctccgcgccgcctcgcgccgcccg | |
| | | ccccggctctgactgaccgcgttactcccacaggtgagcgggcgggacggcccttct | |
| | | cctccgggctgtaattagcgcttggtttaatgacggctcgtttcttttctgtggctgcgtga | |
| | | aagccttaaagggctccgggagggccctttgtgcgggggggagcggctcgggggg | |
| | | tgcgtgcgtgtgtgtgtgcgtggggagcgccgcgtgcggcccgcgctgcccggcg | |
| | | gctgtgagcgctgcgggcgcggcgcggggctttgtgcgctccgcgtgtgcgcgag | |
| | | gggagcgcggccgggggcggtgccccgcggtggggggggctgcgaggggaa | |
| | | caaaggctgcgtgcggggtgtgtgcgtgggggggtgagcagggggtgtgggcgc | |
| | | ggcggtcgggctgtaacccccccctgcacccccctccccgagttgctgagcacggc | |
| | | ccggcttcgggtgcggggctccgtgcggggcgtggcgcggggctcgccgtgccgg | |
| | | gcggggggtggcggcaggtgggggtgccgggcggggcggggccgcctcgggc | |
| | | cggggagggctcgggggaggggcgcggcggccccggagcgccggcggctgtc | |
| | | gaggcgcggcgagccgcagccattgccttttatggtaatcgtgcgagagggcgcag | |
| | | ggacttcctttgtcccaaatctggcggagccgaaatctgggaggcgccgccgcaccc | |
| | | cctctagcgggcgcgggcgaagcggtgcggcgccggcaggaaggaaatgggcg | |
| | | gggagggccttcgtgcgtcgccgcgccgccgtccccttctccatctccagcctcggg | |
| | | gctgccgcagggggacggctgccttcgggggggacggggcagggcggggttcgg | |
| | | cttctggcgtgtgaccggcggctctagagcctctgctaaccatgttcatgccttcttctttt | |
| | | tcctacaggggggatccgtttatctgcagaattcgcccttgacgtcgccaccatggaaa | |
| | | ctctttctaatgcaagtggtacttttgccatacgccttttaaagatactgtgtcaagataac | |
| | | ccttcgcacaacgtgttctgttctcctgtgagcatctcctctgccctggccatggttctcct | |
| | | aggggcaaagggaaacaccgcaacccagatggcccaggcactgtctttaaacacag | |
| | | aggaagacattcatcgggctttccagtcgcttctcactgaagtgaacaaggctggcac | |
| | | acagtacctgctgagaacggccaacaggctctttggagagaaaacttgtcagttcctct | |
| | | caacgtttaaggaatcctgtcttcaattctaccatgctgagctgaaggagctttcctttatc | |
| | | agagctgcagaagagtccaggaaacacatcaacacctgggtctcaaaaaagaccga | |
| | | aggtaaaattgaagagttgttgccgggtagctcaattgatgcagaaaccaggctggttc | |
| | | ttgtcaatgccatctacttcaaaggaaagtggaatgaaccgtttgacgaaacatacaca | |
| | | agggaaatgccctttaaaataaaccaggaggagcaaaggccagtgcagatgatgtat | |
| | | caggaggccacgtttaagctcgcccacgtgggcgaggtgcgcgcgcagctgctgg | |
| | | agctgccctacgccaggaaggagctgagcctgctggtgctgctgcctgacgacggc | |
| | | gtggagctcagcacggtggaaaaaagtctcacttttgagaaactcacagcctggacc | |
| | | aagccagactgtatgaagagtactgaggttgaagttctccttccaaaatttaaactacaa | |
| | | gaggattatgacatggaatctgtgcttcggcatttgggaattgttgatgccttccaacag | |
| | | ggcaaggctgacttgtcggcaatgtcagcggagagagacctgtgtctgtccaagttcg | |
| | | tgcacaagagttttgtggaggtgaatgaagaaggcaccgaggcagcggcagcgtcg | |
| | | agctgctttgtagttgcagagtgctgcatggaatctggccccaggttctgtgctgacca | |
| | | ccctttccttttcttcatcaggcacaacagagccaacagcattctgttctgtggcaggttc | |
| | | tcatcgccaggaagcggagctactaacttcagcctgctgaagcaggctggagacgtg | |
| | | gaggagaaccctggacctatggactggacctggatcctgttcctggtggccgccgcc | |
| | | accagggtgcacagcggcattcatgtcttcattttgggctgtttcagtgcagggcttcct | |
| | | aaaacagaagccaactgggtgaatgtaataagtgatttgaaaaaaattgaagatcttatt | |
| | | caatctatgcatattgatgctactttatatacggaaagtgatgttcaccccagttgcaaag | |
| | | taacagcaatgaagtgctttctcttggagttacaagttatttcacttgagtccggagatgc | |
| | | aagtattcatgatacagtagaaaatctgatcatcctagcaaacaacagtttgtcttctaat | |
| | | gggaatgtaacagaatctggatgcaaagaatgtgaggaactggaggaaaaaaatatt | |
| | | aaagaatttttgcagagttttgtacatattgtccaaatgttcatcaacacttctagcggcgg | |
| | | cggcagcggcggcggcggcagcggcggcggcggcagcggcggcggcggcag | |
| | | cggcggcggcagcctgcagatcacgtgccctccccccatgtccgtggaacacgcag | |
| | | acatctgggtcaagagctacagcttgtactccagggagcggtacatttgtaactctggtt | |
| | | tcaagcgtaaagccggcacgtccagcctgacggagtgcgtgttgaacaaggccacg | |
| | | aatgtcgcccactggacaacccccagtctcaaatgcattagagaccctgccctggttc | |
| | | accaaaggccagcgccaccctccacagtaacgacggcaggggtgaccccacagcc | |
| | | agagagcctctccccttctggaaaagagcccgcagcttcatctcccagctcaaacaac | |
| | | acagcggccacaacagcagctattgtcccgggctcccagctgatgccttcaaaatca | |
| | | ccttccacaggaaccacagagataagcagtcatgagtcctcccacggcaccccctct | |
| | | cagacaacagccaagaactgggaactcacagcatccgcctcccaccagccgccag | |
| | | gtgtgtatccacagggccacagcgacaccactgtggctatctccacgtccactgtcct | |
| | | gctgtgtgggctgagcgctgtgtctctcctggcatgctacctcaagtcaaggcaaactc | |
| | | ccccgctggccagcgttgaaatggaagccatggaggctctgccggtgacttggggg | |
| | | accagcagcagagatgaagacttggaaaactgctctcaccacctatgataaccgctg | |
| | | atcagcctcgactgtgccttctagttgccagccatctgttgtttgcccctcccccgtgcct | |
| | | tccttgaccctggaaggtgccactcccactgtcctttcctaataaaatgaggaaattgca | |
| | | tcgcattgtctgagtaggtgtcattctattctggggggtggggtggggcaggacagca | |
| | | agggggaggattgggaagacaatagcaggcatgctggggatgcggtgggctctatg | |
| | | ggtcgacccagcgtgagtctctcctaccctcccgctctggtccttcctctcccgctctgc | |
| | | accctctgtggccctcgctgtgctctctcgctccgtgacttcccttctccaagttctccttg | |
| | | gtggcccgccgtggggctagtccagggctggatctcggggaagcggcggggtggc | |
| | | ctgggagtggggaagggggtgcgcacccgggacgcgcgctacttgcccctttcggc | |
| | | ggggagcaggggagacctttggcctacggcgacgggagggtcgggacaaagttta | |
| | | gggcgtcgataagcgtcagagcgccgaggttgggggagggtttctcttccgctctttc | |
| | | gcggggcctctggctcccccagcgcagctggagtgggggacgggtaggctcgtcc | |
| | | caaaggcgcggcgctgaggtttgtgaacgcgtggaggggcgcttggggtctgggg | |
| | | gaggcgtcgcccgggtaagcctgtctgctgcggctctgcttcccttagactggagagc | |
| | | tgtggacttcgtctaggcgcccgctaagttcgcatgtcctagcacctctgggtctatgtg | |
| | | gggccacaccgtggggaggaaacagcacgcgacgtttgtagaatgcttggctgtgat | |
| | | acaaagcggtttcgaataattaacttatttgttcccatcacatgtcacttttaaaaaattata | |
| | | agaactacccgttattgacatctttctgtgtgccaaggactttatgtgctttgcgtcatttaa | |
| | | ttttgaaaacagttatcttccgccatagataactactatggttatcttctggtaaccacgtg | |
| | | cggaccgaggctgcagcgtcgtcctccctaggaacccctagtgatggagttggccac | |
| | | tccctctctgcgcgctcgctcgctcactgaggccgggcgaccaaaggtcgcccgac | |
| | | gcccgggctttgcccgggcggcctcagtgagcgagcgagcgcgcagctgcctgca | |
| | | ggggcgcctgatgcggtattttctccttacgcatctgtgcggtatttcacaccgcatacg | |
| | | tcaaagcaaccatagtacgcgccctgtagcggcgcattaagcgcggcgggtgtggt | |
| | | ggttacgcgcagcgtgaccgctacacttgccagcgccctagcgcccgctcctttcgct | |
| | | ttcttcccttcctttctcgccacgttcgccggctttccccgtcaagctctaaatcgggggc | |
| | | tccctttagggttccgatttagtgctttacggcacctcgaccccaaaaaacttgatttggg | |
| | | tgatggttcacgtagtgggccatcgccctgatagacggtttttcgccctttgacgttgga | |
| | | gtccacgttctttaatagtggactcttgttccaaactggaacaacactcaaccctatctcg | |
| | | ggctattcttttgatttataagggattttgccgatttcggcctattggttaaaaaatgagctg | |
| | | atttaacaaaaatttaacgcgaattttaacaaaatattaacgtttacaattttatggtgcact | |
| | | ctcagtacaatctgctctgatgccgcatagttaagccagccccgacacccgccaacac | |
| | | ccgctgacgcgccctgacgggcttgtctgctcccggcatccgcttacagacaagctgt | |
| | | gaccgtctccgggagctgcatgtgtcagaggttttcaccgtcatcaccgaaacgcgcg | |
| | | agacgaaagggcctcgtgatacgcctatttttataggttaatgtcatgaacaataaaact | |
| | | gtctgcttacataaacagtaatacaaggggtgttatgagccatattcaacgggaaacgt | |
| | | cgaggccgcgattaaattccaacatggatgctgatttatatgggtataaatgggctcgc | |
| | | gataatgtcgggcaatcaggtgcgacaatctatcgcttgtatgggaagcccgatgcgc | |
| | | cagagttgtttctgaaacatggcaaaggtagcgttgccaatgatgttacagatgagatg | |
| | | gtcagactaaactggctgacggaatttatgcctcttccgaccatcaagcattttatccgta | |
| | | ctcctgatgatgcatggttactcaccactgcgatccccggaaaaacagcattccaggta | |
| | | ttagaagaatatcctgattcaggtgaaaatattgttgatgcgctggcagtgttcctgcgc | |
| | | cggttgcattcgattcctgtttgtaattgtccttttaacagcgatcgcgtatttcgtctcgct | |
| | | caggcgcaatcacgaatgaataacggtttggttgatgcgagtgattttgatgacgagc | |
| | | gtaatggctggcctgttgaacaagtctggaaagaaatgcataaacttttgccattctcac | |
| | | cggattcagtcgtcactcatggtgatttctcacttgataaccttatttttgacgaggggaa | |
| | | attaataggttgtattgatgttggacgagtcggaatcgcagaccgataccaggatcttg | |
| | | ccatcctatggaactgcctcggtgagttttctccttcattacagaaacggctttttcaaaa | |
| | | atatggtattgataatcctgatatgaataaattgcagtttcatttgatgctcgatgagtttttc | |
| | | taatctcatgaccaaaatcccttaacgtgagttttcgttccactgagcgtcagaccccgt | |
| | | agaaaagatcaaaggatcttcttgagatcctttttttctgcgcgtaatctgctgcttgcaaa | |
| | | caaaaaaaccaccgctaccagcggtggtttgtttgccggatcaagagctaccaactctt | |
| | | tttccgaaggtaactggcttcagcagagcgcagataccaaatactgtccttctagtgta | |
| | | gccgtagttaggccaccacttcaagaactctgtagcaccgcctacatacctcgctctgc | |
| | | taatcctgttaccagtggctgctgccagtggcgataagtcgtgtcttaccgggttggact | |
| | | caagacgatagttaccggataaggcgcagcggtcgggctgaacggggggttcgtgc | |
| | | acacagcccagcttggagcgaacgacctacaccgaactgagatacctacagcgtga | |
| | | gctatgagaaagcgccacgcttcccgaagggagaaaggcggacaggtatccggta | |
| | | agcggcagggtcggaacaggagagcgcacgagggagcttccagggggaaacgc | |
| | | ctggtatctttatagtcctgtcgggtttcgccacctctgacttgagcgtcgatttttgtgatg | |
| | | ctcgtcaggggggcggagcctatggaaaaacgccagcaacgcggcctttttacggtt | |
| | | cctggccttttgctggccttttgctcacatgt | |
| SERPIN | | atggaaactctttctaatgcaagtggtacttttgccatacgccttttaaagatactgtgtca | 137 |
| B9-P2A- | | agataacccttcgcacaacgtgttctgttctcctgtgagcatctcctctgccctggccat | |
| IL15/IL15 | | ggttctcctaggggcaaagggaaacaccgcaacccagatggcccaggcactgtcttt | |
| Rα | | aaacacagaggaagacattcatcgggctttccagtcgcttctcactgaagtgaacaag | |
| transgene | | gctggcacacagtacctgctgagaacggccaacaggctctttggagagaaaacttgt | |
| | | cagttcctctcaacgtttaaggaatcctgtcttcaattctaccatgctgagctgaaggag | |
| | | ctttcctttatcagagctgcagaagagtccaggaaacacatcaacacctgggtctcaaa | |
| | | aaagaccgaaggtaaaattgaagagttgttgccgggtagctcaattgatgcagaaacc | |
| | | aggctggttcttgtcaatgccatctacttcaaaggaaagtggaatgaaccgtttgacga | |
| | | aacatacacaagggaaatgccctttaaaataaaccaggaggagcaaaggccagtgc | |
| | | agatgatgtatcaggaggccacgtttaagctcgcccacgtgggcgaggtgcgcgcg | |
| | | cagctgctggagctgccctacgccaggaaggagctgagcctgctggtgctgctgcct | |
| | | gacgacggcgtggagctcagcacggtggaaaaaagtctcacttttgagaaactcaca | |
| | | gcctggaccaagccagactgtatgaagagtactgaggttgaagttctccttccaaaatt | |
| | | taaactacaagaggattatgacatggaatctgtgcttcggcatttgggaattgttgatgc | |
| | | cttccaacagggcaaggctgacttgtcggcaatgtcagcggagagagacctgtgtct | |
| | | gtccaagttcgtgcacaagagttttgtggaggtgaatgaagaaggcaccgaggcagc | |
| | | ggcagcgtcgagctgctttgtagttgcagagtgctgcatggaatctggccccaggttct | |
| | | gtgctgaccaccctttccttttcttcatcaggcacaacagagccaacagcattctgttctg | |
| | | tggcaggttctcatcgccaggaagcggagctactaacttcagcctgctgaagcaggct | |
| | | ggagacgtggaggagaaccctggacctatggactggacctggatcctgttcctggtg | |
| | | gccgccgccaccagggtgcacagcggcattcatgtcttcattttgggctgtttcagtgc | |
| | | agggcttcctaaaacagaagccaactgggtgaatgtaataagtgatttgaaaaaaattg | |
| | | aagatcttattcaatctatgcatattgatgctactttatatacggaaagtgatgttcacccc | |
| | | agttgcaaagtaacagcaatgaagtgctttctcttggagttacaagttatttcacttgagt | |
| | | ccggagatgcaagtattcatgatacagtagaaaatctgatcatcctagcaaacaacagt | |
| | | ttgtcttctaatgggaatgtaacagaatctggatgcaaagaatgtgaggaactggagga | |
| | | aaaaaatattaaagaatttttgcagagttttgtacatattgtccaaatgttcatcaacacttc | |
| | | tagcggcggcggcagcggcggcggcggcagcggcggcggcggcagcggcggc | |
| | | ggcggcagcggcggcggcagcctgcagatcacgtgccctccccccatgtccgtgg | |
| | | aacacgcagacatctgggtcaagagctacagcttgtactccagggagcggtacatttg | |
| | | taactctggtttcaagcgtaaagccggcacgtccagcctgacggagtgcgtgttgaac | |
| | | aaggccacgaatgtcgcccactggacaacccccagtctcaaatgcattagagaccct | |
| | | gccctggttcaccaaaggccagcgccaccctccacagtaacgacggcaggggtga | |
| | | ccccacagccagagagcctctccccttctggaaaagagcccgcagcttcatctccca | |
| | | gctcaaacaacacagcggccacaacagcagctattgtcccgggctcccagctgatgc | |
| | | cttcaaaatcaccttccacaggaaccacagagataagcagtcatgagtcctcccacgg | |
| | | caccccctctcagacaacagccaagaactgggaactcacagcatccgcctcccacc | |
| | | agccgccaggtgtgtatccacagggccacagcgacaccactgtggctatctccacgt | |
| | | ccactgtcctgctgtgtgggctgagcgctgtgtctctcctggcatgctacctcaagtcaa | |
| | | ggcaaactcccccgctggccagcgttgaaatggaagccatggaggctctgccggtg | |
| | | acttgggggaccagcagcagagatgaagacttggaaaactgctctcaccacctatga | |
| | | taaccgctgatcagcctcgactgtgccttctagttgccagccatctgttgtttgcccctcc | |
| | | cccgtgccttccttgaccctggaaggtgccactcccactgtcctttcctaataaaatgag | |
| | | gaaattgcatcgcattgtctgagtaggtgtcattctattctggggggtggggtggggca | |
| | | ggacagcaagggggaggattgggaagacaatagcaggcatgctggggatgcggtg | |
| | | ggctctatgg | |
| CAG- | Transgene + | gacattgattattgactagttattaatagtaatcaattacggggtcattagttcatagccca | 138 |
| SERPINB9- | promoter | tatatggagttccgcgttacataacttacggtaaatggcccgcctggctgaccgcccaa | |
| P2A- | | cgacccccgcccattgacgtcaataatgacgtatgttcccatagtaacgccaataggg | |
| IL15/IL15 | | actttccattgacgtcaatgggtggactatttacggtaaactgcccacttggcagtacat | |
| Rα | | caagtgtatcatatgccaagtacgccccctattgacgtcaatgacggtaaatggcccg | |
| | | cctggcattatgcccagtacatgaccttatgggactttcctacttggcagtacatctacgt | |
| | | attagtcatcgctattaccatgggtcgaggtgagccccacgttctgcttcactctcccca | |
| | | tctcccccccctccccacccccaattttgtatttatttattttttaattattttgtgcagcgatg | |
| | | ggggcggggggggggggggcgcgcgccaggcggggcggggcggggcgagg | |
| | | ggcggggcggggcgaggcggagaggtgcggcggcagccaatcagagcggcgc | |
| | | gctccgaaagtttccttttatggcgaggcggcggcggcggcggccctataaaaagcg | |
| | | aagcgcgcggcgggcgggagtcgctgcgttgccttcgccccgtgccccgctccgc | |
| | | gccgcctcgcgccgcccgccccggctctgactgaccgcgttactcccacaggtgag | |
| | | cgggcgggacggcccttctcctccgggctgtaattagcgcttggtttaatgacggctc | |
| | | gtttcttttctgtggctgcgtgaaagccttaaagggctccgggagggccctttgtgcgg | |
| | | gggggagcggctcggggggtgcgtgcgtgtgtgtgtgcgtggggagcgccgcgtg | |
| | | cggcccgcgctgcccggcggctgtgagcgctgcgggcgcggcgcggggctttgtg | |
| | | cgctccgcgtgtgcgcgaggggagcgcggccgggggcggtgccccgcggtgcg | |
| | | ggggggctgcgaggggaacaaaggctgcgtgcggggtgtgtgcgtgggggggtg | |
| | | agcagggggtgtgggcgcggcggtcgggctgtaacccccccctgcacccccctcc | |
| | | ccgagttgctgagcacggcccggcttcgggtgcggggctccgtgcggggcgtggc | |
| | | gcggggctcgccgtgccgggcggggggtggcggcaggtgggggtgccgggcgg | |
| | | ggcggggccgcctcgggccggggagggctcgggggaggggcgcggcggcccc | |
| | | ggagcgccggcggctgtcgaggcgcggcgagccgcagccattgccttttatggtaat | |
| | | cgtgcgagagggcgcagggacttcctttgtcccaaatctggcggagccgaaatctgg | |
| | | gaggcgccgccgcaccccctctagcgggcgcgggcgaagcggtgcggcgccgg | |
| | | caggaaggaaatgggcggggagggccttcgtgcgtcgccgcgccgccgtcccctt | |
| | | ctccatctccagcctcggggctgccgcagggggacggctgccttcgggggggacg | |
| | | gggcagggcggggttcggcttctggcgtgtgaccggcggctctagagcctctgctaa | |
| | | ccatgttcatgccttcttctttttcctacaggggggatccgtttatctgcagaattcgccctt | |
| | | gacgtcgccaccatggaaactctttctaatgcaagtggtacttttgccatacgccttttaa | |
| | | agatactgtgtcaagataacccttcgcacaacgtgttctgttctcctgtgagcatctcctc | |
| | | tgccctggccatggttctcctaggggcaaagggaaacaccgcaacccagatggccc | |
| | | aggcactgtctttaaacacagaggaagacattcatcgggctttccagtcgcttctcactg | |
| | | aagtgaacaaggctggcacacagtacctgctgagaacggccaacaggctctttgga | |
| | | gagaaaacttgtcagttcctctcaacgtttaaggaatcctgtcttcaattctaccatgctg | |
| | | agctgaaggagctttcctttatcagagctgcagaagagtccaggaaacacatcaacac | |
| | | ctgggtctcaaaaaagaccgaaggtaaaattgaagagttgttgccgggtagctcaatt | |
| | | gatgcagaaaccaggctggttcttgtcaatgccatctacttcaaaggaaagtggaatga | |
| | | accgtttgacgaaacatacacaagggaaatgccctttaaaataaaccaggaggagca | |
| | | aaggccagtgcagatgatgtatcaggaggccacgtttaagctcgcccacgtgggcga | |
| | | ggtgcgcgcgcagctgctggagctgccctacgccaggaaggagctgagcctgctg | |
| | | gtgctgctgcctgacgacggcgtggagctcagcacggtggaaaaaagtctcacttttg | |
| | | agaaactcacagcctggaccaagccagactgtatgaagagtactgaggttgaagttct | |
| | | ccttccaaaatttaaactacaagaggattatgacatggaatctgtgcttcggcatttggg | |
| | | aattgttgatgccttccaacagggcaaggctgacttgtcggcaatgtcagcggagaga | |
| | | gacctgtgtctgtccaagttcgtgcacaagagttttgtggaggtgaatgaagaaggca | |
| | | ccgaggcagcggcagcgtcgagctgctttgtagttgcagagtgctgcatggaatctg | |
| | | gccccaggttctgtgctgaccaccctttccttttcttcatcaggcacaacagagccaaca | |
| | | gcattctgttctgtggcaggttctcatcgccaggaagcggagctactaacttcagcctg | |
| | | ctgaagcaggctggagacgtggaggagaaccctggacctatggactggacctggat | |
| | | cctgttcctggtggccgccgccaccagggtgcacagcggcattcatgtcttcattttgg | |
| | | gctgtttcagtgcagggcttcctaaaacagaagccaactgggtgaatgtaataagtgat | |
| | | ttgaaaaaaattgaagatcttattcaatctatgcatattgatgctactttatatacggaaagt | |
| | | gatgttcaccccagttgcaaagtaacagcaatgaagtgctttctcttggagttacaagtt | |
| | | atttcacttgagtccggagatgcaagtattcatgatacagtagaaaatctgatcatccta | |
| | | gcaaacaacagtttgtcttctaatgggaatgtaacagaatctggatgcaaagaatgtga | |
| | | ggaactggaggaaaaaaatattaaagaatttttgcagagttttgtacatattgtccaaatg | |
| | | ttcatcaacacttctagcggcggcggcagcggcggcggcggcagcggcggcggc | |
| | | ggcagcggcggcggcggcagcggcggcggcagcctgcagatcacgtgccctccc | |
| | | cccatgtccgtggaacacgcagacatctgggtcaagagctacagcttgtactccagg | |
| | | gagcggtacatttgtaactctggtttcaagcgtaaagccggcacgtccagcctgacgg | |
| | | agtgcgtgttgaacaaggccacgaatgtcgcccactggacaacccccagtctcaaat | |
| | | gcattagagaccctgccctggttcaccaaaggccagcgccaccctccacagtaacga | |
| | | cggcaggggtgaccccacagccagagagcctctccccttctggaaaagagcccgc | |
| | | agcttcatctcccagctcaaacaacacagcggccacaacagcagctattgtcccggg | |
| | | ctcccagctgatgccttcaaaatcaccttccacaggaaccacagagataagcagtcat | |
| | | gagtcctcccacggcaccccctctcagacaacagccaagaactgggaactcacagc | |
| | | atccgcctcccaccagccgccaggtgtgtatccacagggccacagcgacaccactgt | |
| | | ggctatctccacgtccactgtcctgctgtgtgggctgagcgctgtgtctctcctggcatg | |
| | | ctacctcaagtcaaggcaaactcccccgctggccagcgttgaaatggaagccatgga | |
| | | ggctctgccggtgacttgggggaccagcagcagagatgaagacttggaaaactgct | |
| | | ctcaccacctatgataaccgctgatcagcctcgactgtgccttctagttgccagccatct | |
| | | gttgtttgcccctcccccgtgccttccttgaccctggaaggtgccactcccactgtccttt | |
| | | cctaataaaatgaggaaattgcatcgcattgtctgagtaggtgtcattctattctggggg | |
| | | gtggggtggggcaggacagcaagggggaggattgggaagacaatagcaggcatg | |
| | | ctggggatgcggtgggctctatgg | |
| CAG- | Transgene + | gacattgattattgactagttattaatagtaatcaattacggggtcattagttcatagc | 139 |
| CD30 CAR | promoter | ccatatatggagttccgcgttacataacttacggtaaatggcccgcctggctgaccgc | |
| 4-P2A- | | ccaacgacccccgcccattgacgtcaataatgacgtatgttcccatagtaacgccaa | |
| HLA-E | | tagggactttccattgacgtcaatgggtggactatttacggtaaactgcccacttggc | |
| | | agtacatcaagtgtatcatatgccaagtacgccccctattgacgtcaatgacggtaa | |
| | | atggcccgcctggcattatgcccagtacatgaccttatgggactttcctacttggcag | |
| | | tacatctacgtattagtcatcgctattaccatgggtcgaggtgagccccacgttctgct | |
| | | tcactctccccatctcccccccctccccacccccaattttgtatttatttattttttaatta | |
| | | ttttgtgcagcgatgggggcggggggggggggggcgcgcgccaggcggggcgggg | |
| | | cggggcgaggggcggggcggggcgaggcggagaggtgcggcggcagccaatcag | |
| | | agcggcgcgctccgaaagtttccttttatggcgaggcggcggcggcggcggccctat | |
| | | aaaaagcgaagcgcgcggcgggcgggagtcgctgcgttgccttcgccccgtgcccc | |
| | | gctccgcgccgcctcgcgccgcccgccccggctctgactgaccgcgttactcccaca | |
| | | ggtgagcgggcgggacggcccttctcctccgggctgtaattagcgcttggtttaatga | |
| | | cggctcgtttcttttctgtggctgcgtgaaagccttaaagggctccgggagggcccttt | |
| | | gtgcgggggggagcggctcggggggtgcgtgcgtgtgtgtgtgcgtggggagcgcc | |
| | | gcgtgcggcccgcgctgcccggcggctgtgagcgctgcgggcgcggcgcggggctt | |
| | | tgtgcgctccgcgtgtgcgcgaggggagcgcggccgggggcggtgccccgcggtgc | |
| | | gggggggctgcgaggggaacaaaggctgcgtgcggggtgtgtgcgtgggggggtg | |
| | | agcagggggtgtgggcgcggcggtcgggctgtaacccccccctgcacccccctccc | |
| | | cgagttgctgagcacggcccggcttcgggtgcggggctccgtgcggggcgtggcgc | |
| | | ggggctcgccgtgccgggcggggggtggcggcaggtgggggtgccgggcggggcg | |
| | | gggccgcctcgggccggggagggctcgggggaggggcgcggcggccccggagcg | |
| | | ccggcggctgtcgaggcgcggcgagccgcagccattgccttttatggtaatcgtgcg | |
| | | agagggcgcagggacttcctttgtcccaaatctggcggagccgaaatctgggaggc | |
| | | gccgccgcaccccctctagcgggcgcgggcgaagcggtgcggcgccggcaggaag | |
| | | gaaatgggcggggagggccttcgtgcgtcgccgcgccgccgtccccttctccatctc | |
| | | cagcctcggggctgccgcagggggacggctgccttcgggggggacggggcagggc | |
| | | ggggttcggcttctggcgtgtgaccggcggctctagagcctctgctaaccatgttcat | |
| | | gccttcttctttttcctacaggggggatccgtttatctgcagaattcgcccttgacgtcg | |
| | | ccaccatggcgcttccggtgacagcactgctcctccccttggcgctgttgctccacgc | |
| | | agcaaggccgcagatccagctgcagcagagcggccccgaggtggtgaagcccggc | |
| | | gccagcgtgaagatcagctgcaaggccagcggctacaccttcaccgactactacat | |
| | | cacctgggtgaagcagaagcccggccagggcctggagtggatcggctggatctacc | |
| | | ccggcagcggcaacaccaagtacaacgagaagttcaagggcaaggccaccctgac | |
| | | cgtggacaccagcagcagcaccgccttcatgcagctgagcagcctgaccagcgagg | |
| | | acaccgccgtgtacttctgcgccaactacggcaactactggttcgcctactggggcc | |
| | | agggcacccaggtgaccgtgagcgccggcggcggcggcagcggcggcggcggca | |
| | | gcggcggcggcggcagcgacatcgtgctgacccagagccccgccagcctggccgtg | |
| | | agcctgggccagagagccaccatcagctgcaaggccagccagagcgtggacttcg | |
| | | acggcgacagctacatgaactggtaccagcagaagcccggccagccccccaaggt | |
| | | gctgatctacgccgccagcaacctggagagcggcatccccgccagattcagcggca | |
| | | gcggcagcggcaccgacttcaccctgaacatccaccccgtggaggaggaggacgc | |
| | | cgccacctactactgccagcagagcaacgaggacccctggaccttcggcggcggca | |
| | | ccaagctggagatcaagagcgccgccgccttcgtgcccgtgttcctgcccgccaagc | |
| | | ccaccaccacccccgcccccagaccccccacccccgcccccaccatcgccagccag | |
| | | cccctgagcctgagacccgaggcctgcagacccgccgccggcggcgccgtgcacac | |
| | | cagaggcctggacttcgcctgcgacatctacatctgggcccccctggccggcacctg | |
| | | cggcgtgctgctgctgagcctggtgatcaccctgtactgcaaccacagaaacagaa | |
| | | gcaagagaagcagactgctgcacagcgactacatgaacatgacccccagaagacc | |
| | | cggccccaccagaaagcactaccagccctacgccccccccagagacttcgccgcct | |
| | | acagaagcagagtgaagttcagcagaagcgccgacgcccccgcctaccagcaggg | |
| | | ccagaaccagctgtacaacgagctgaacctgggcagaagagaggagtacgacgtg | |
| | | ctggacaagagaagaggcagagaccccgagatgggcggcaagcccagaagaaa | |
| | | gaacccccaggagggcctgtacaacgagctgcagaaggacaagatggccgaggcc | |
| | | tacagcgagatcggcatgaagggcgagagaagaagaggcaagggccacgacggc | |
| | | ctgtaccagggcctgagcaccgccaccaaggacacctacgacgccctgcacatgca | |
| | | ggccctgccccccagaggaagcggagctactaacttcagcctgctgaagcaggctg | |
| | | gagacgtggaggagaaccctggacctatgtctcgctccgttgccttagctgtgctcgc | |
| | | gctactctctctttctggattagaggctgtcatggcgccccgaaccctcttcctgggtg | |
| | | gaggcggttcaggcggaggtggctctggcggtggcggatcgatccagcgtactcca | |
| | | aagattcaggtttactcacgtcatccagcagagaatggaaagtcaaatttcctgaat | |
| | | tgctatgtgtctgggtttcatccatccgacattgaagttgacttactgaagaatggag | |
| | | agagaattgaaaaagtggagcattcagacttgtctttcagcaaggactggtctttcta | |
| | | tctcttgtactacactgaattcacccccactgaaaaagatgagtatgcctgccgtgtg | |
| | | aaccatgtgactttgtcacagcccaagatagttaagtgggatcgagacatgggtggt | |
| | | ggtggttctggtggtggtggttctggcggcggcggctccggtggtggtggatccggct | |
| | | cccactccttgaagtatttccacacttccgtgtcccggcccggccgcggggagccccg | |
| | | cttcatctctgtgggctacgtggacgacacccagttcgtgcgcttcgacaacgacgcc | |
| | | gcgagtccgaggatggtgccgcgggcgccgtggatggagcaggaggggtcagagt | |
| | | attgggaccgggagacacggagcgccagggacaccgcacagattttccgagtgaa | |
| | | tctgcggacgctgcgcggctactacaatcagagcgaggccgggtctcacaccctgc | |
| | | agtggatgcatggctgcgagctggggcccgacgggcgcttcctccgcgggtatgaa | |
| | | cagttcgcctacgacggcaaggattatctcaccctgaatgaggacctgcgctcctgg | |
| | | accgcggtggacacggcggctcagatctccgagcaaaagtcaaatgatgcctctga | |
| | | ggcggagcaccagagagcctacctggaagacacatgcgtggagtggctccacaaa | |
| | | tacctggagaaggggaaggagacgctgcttcacctggagcccccaaagacacacg | |
| | | tgactcaccaccccatctctgaccatgaggccaccctgaggtgctgggccctgggctt | |
| | | ctaccctgcggagatcacactgacctggcagcaggatggggagggccatacccagg | |
| | | acacggagctcgtggagaccaggcctgcaggggatggaaccttccagaagtgggc | |
| | | agctgtggtggtgccttctggagaggagcagagatacacgtgccatgtgcagcatg | |
| | | aggggctacccgagcccgtcaccctgagatggaagccggcttcccagcccaccatc | |
| | | cccatcgtgggcatcattgctggcctggttctccttggatctgtggtctctggagctgt | |
| | | ggttgctgctgtgatatggaggaagaagagctcaggtggaaaaggagggagctact | |
| | | ctaaggctgagtggagcgacagtgcccaggggtctgagtctcacagcttg | |
| CAG- | Transgene + | gacattgattattgactagttattaatagtaatcaattacggggtcattagttcatagc | 140 |
| CD30 CAR | promoter | ccatatatggagttccgcgttacataacttacggtaaatggcccgcctggctgaccgc | |
| 5-P2A- | | ccaacgacccccgcccattgacgtcaataatgacgtatgttcccatagtaacgccaa | |
| HLA-E | | tagggactttccattgacgtcaatgggtggactatttacggtaaactgcccacttggc | |
| | | agtacatcaagtgtatcatatgccaagtacgccccctattgacgtcaatgacggtaa | |
| | | atggcccgcctggcattatgcccagtacatgaccttatgggactttcctacttggcag | |
| | | tacatctacgtattagtcatcgctattaccatgggtcgaggtgagccccacgttctgct | |
| | | tcactctccccatctcccccccctccccacccccaattttgtatttatttattttttaatta | |
| | | ttttgtgcagcgatgggggcggggggggggggggcgcgcgccaggcggggcgggg | |
| | | cggggcgaggggcggggcggggcgaggcggagaggtgcggcggcagccaatcag | |
| | | agcggcgcgctccgaaagtttccttttatggcgaggcggcggcggcggcggccctat | |
| | | aaaaagcgaagcgcgcggcgggcgggagtcgctgcgttgccttcgccccgtgcccc | |
| | | gctccgcgccgcctcgcgccgcccgccccggctctgactgaccgcgttactcccaca | |
| | | ggtgagcgggcgggacggcccttctcctccgggctgtaattagcgcttggtttaatga | |
| | | cggctcgtttcttttctgtggctgcgtgaaagccttaaagggctccgggagggcccttt | |
| | | gtgcgggggggagcggctcggggggtgcgtgcgtgtgtgtgtgcgtggggagcgcc | |
| | | gcgtgcggcccgcgctgcccggcggctgtgagcgctgcgggcgcggcgcggggctt | |
| | | tgtgcgctccgcgtgtgcgcgaggggagcgcggccgggggcggtgccccgcggtgc | |
| | | gggggggctgcgaggggaacaaaggctgcgtgcggggtgtgtgcgtgggggggtg | |
| | | agcagggggtgtgggcgcggcggtcgggctgtaacccccccctgcacccccctccc | |
| | | cgagttgctgagcacggcccggcttcgggtgcggggctccgtgcggggcgtggcgc | |
| | | ggggctcgccgtgccgggcggggggtggcggcaggtgggggtgccgggcggggcg | |
| | | gggccgcctcgggccggggagggctcgggggaggggcgcggcggccccggagcg | |
| | | ccggcggctgtcgaggcgcggcgagccgcagccattgccttttatggtaatcgtgcg | |
| | | agagggcgcagggacttcctttgtcccaaatctggcggagccgaaatctgggaggc | |
| | | gccgccgcaccccctctagcgggcgcgggcgaagcggtgcggcgccggcaggaag | |
| | | gaaatgggcggggagggccttcgtgcgtcgccgcgccgccgtccccttctccatctc | |
| | | cagcctcggggctgccgcagggggacggctgccttcgggggggacggggcagggc | |
| | | ggggttcggcttctggcgtgtgaccggcggctctagagcctctgctaaccatgttcat | |
| | | gccttcttctttttcctacaggggggatccgtttatctgcagaattcgcccttgacgtcg | |
| | | ccaccatggcgcttccggtgacagcactgctcctccccttggcgctgttgctccacgc | |
| | | agcaaggccgcaggtgcagctgcagcagtggggcgccggcctgctgaagcccagc | |
| | | gagaccctgagcctgacctgcgccgtgtacggcggcagcttcagcgcctactactgg | |
| | | agctggatcagacagccccccggcaagggcctggagtggatcggcgacatcaacc | |
| | | acggcggcggcaccaactacaaccccagcctgaagagcagagtgaccatcagcgt | |
| | | ggacaccagcaagaaccagttcagcctgaagctgaacagcgtgaccgccgccgac | |
| | | accgccgtgtactactgcgccagcctgaccgcctactggggccagggcagcctggtg | |
| | | accgtgagcagcggcggcggcggcagcggcggcggcggcagcggcggcggcggc | |
| | | agcgacatccagatgacccagagccccaccagcctgagcgccagcgtgggcgaca | |
| | | gagtgaccatcacctgcagagccagccagggcatcagcagctggctgacctggtac | |
| | | cagcagaagcccgagaaggcccccaagagcctgatctacgccgccagcagcctgc | |
| | | agagcggcgtgcccagcagattcagcggcagcggcagcggcaccgacttcaccctg | |
| | | accatcagcagcctgcagcccgaggacttcgccacctactactgccagcagtacga | |
| | | cagctaccccatcaccttcggccagggcaccagactggagatcaagagcgccgccg | |
| | | ccttcgtgcccgtgttcctgcccgccaagcccaccaccacccccgcccccagacccc | |
| | | ccacccccgcccccaccatcgccagccagcccctgagcctgagacccgaggcctgc | |
| | | agacccgccgccggcggcgccgtgcacaccagaggcctggacttcgcctgcgacat | |
| | | ctacatctgggcccccctggccggcacctgcggcgtgctgctgctgagcctggtgatc | |
| | | accctgtactgcaaccacagaaacagaaagagaggcagaaagaagctgctgtaca | |
| | | tcttcaagcagcccttcatgagacccgtgcagaccacccaggaggaggacggctgc | |
| | | agctgcagattccccgaggaggaggagggcggctgcgagctgagagtgaagttca | |
| | | gcagaagcgccgacgcccccgcctaccagcagggccagaaccagctgtacaacga | |
| | | gctgaacctgggcagaagagaggagtacgacgtgctggacaagagaagaggcag | |
| | | agaccccgagatgggcggcaagcccagaagaaagaacccccaggagggcctgta | |
| | | caacgagctgcagaaggacaagatggccgaggcctacagcgagatcggcatgaa | |
| | | gggcgagagaagaagaggcaagggccacgacggcctgtaccagggcctgagcac | |
| | | cgccaccaaggacacctacgacgccctgcacatgcaggccctgccccccagagga | |
| | | agcggattcagcctgctgaagcaggctggagacgtggaggagaaccctggacctat | |
| | | gtctcgctccgttgccttagctgtgctcgcgctactctctctttctggattagaggctgt | |
| | | catggcgccccgaaccctcttcctgggtggaggcggttcaggcggaggtggctctgg | |
| | | cggtggcggatcgatccagcgtactccaaagattcaggtttactcacgtcatccagc | |
| | | agagaatggaaagtcaaatttcctgaattgctatgtgtctgggtttcatccatccgac | |
| | | attgaagttgacttactgaagaatggagagagaattgaaaaagtggagcattcaga | |
| | | cttgtctttcagcaaggactggtctttctatctcttgtactacactgaattcacccccac | |
| | | tgaaaaagatgagtatgcctgccgtgtgaaccatgtgactttgtcacagcccaagat | |
| | | agttaagtgggatcgagacatgggtggtggtggttctggtggtggtggttctggcgg | |
| | | cggcggctccggtggtggtggatccggctcccactccttgaagtatttccacacttcc | |
| | | gtgtcccggcccggccgcggggagccccgcttcatctctgtgggctacgtggacgac | |
| | | acccagttcgtgcgcttcgacaacgacgccgcgagtccgaggatggtgccgcgggc | |
| | | gccgtggatggagcaggaggggtcagagtattgggaccgggagacacggagcgcc | |
| | | agggacaccgcacagattttccgagtgaatctgcggacgctgcgcggctactacaat | |
| | | cagagcgaggccgggtctcacaccctgcagtggatgcatggctgcgagctggggcc | |
| | | cgacgggcgcttcctccgcgggtatgaacagttcgcctacgacggcaaggattatct | |
| | | caccctgaatgaggacctgcgctcctggaccgcggtggacacggcggctcagatct | |
| | | ccgagcaaaagtcaaatgatgcctctgaggcggagcaccagagagcctacctgga | |
| | | agacacatgcgtggagtggctccacaaatacctggagaaggggaaggagacgctg | |
| | | cttcacctggagcccccaaagacacacgtgactcaccaccccatctctgaccatgag | |
| | | gccaccctgaggtgctgggccctgggcttctaccctgcggagatcacactgacctgg | |
| | | cagcaggatggggagggccatacccaggacacggagctcgtggagaccaggcctg | |
| | | caggggatggaaccttccagaagtgggcagctgtggtggtgccttctggagaggag | |
| | | cagagatacacgtgccatgtgcagcatgaggggctacccgagcccgtcaccctgag | |
| | | atggaagccggcttcccagcccaccatccccatcgtgggcatcattgctggcctggtt | |
| | | ctccttggatctgtggtctctggagctgtggttgctgctgtgatatggaggaagaaga | |
| | | gctcaggtggaaaaggagggagctactctaaggctgagtggagcgacagtgccca | |
| | | ggggtctgagtctcacagcttg | |
| CAG- | Transgene + | gacattgattattgactagttattaatagtaatcaattacggggtcattagttcatagc | 141 |
| CD30 CAR | promoter | ccatatatggagttccgcgttacataacttacggtaaatggcccgcctggctgaccgc | |
| 6-P2A- | | ccaacgacccccgcccattgacgtcaataatgacgtatgttcccatagtaacgccaa | |
| HLA-E | | tagggactttccattgacgtcaatgggtggactatttacggtaaactgcccacttggc | |
| | | agtacatcaagtgtatcatatgccaagtacgccccctattgacgtcaatgacggtaa | |
| | | atggcccgcctggcattatgcccagtacatgaccttatgggactttcctacttggcag | |
| | | tacatctacgtattagtcatcgctattaccatgggtcgaggtgagccccacgttctgct | |
| | | tcactctccccatctcccccccctccccacccccaattttgtatttatttattttttaatta | |
| | | ttttgtgcagcgatgggggcggggggggggggggcgcgcgccaggcggggcgggg | |
| | | cggggcgaggggcggggcggggcgaggcggagaggtgcggcggcagccaatcag | |
| | | agcggcgcgctccgaaagtttccttttatggcgaggcggcggcggcggcggccctat | |
| | | aaaaagcgaagcgcgcggcgggcgggagtcgctgcgttgccttcgccccgtgcccc | |
| | | gctccgcgccgcctcgcgccgcccgccccggctctgactgaccgcgttactcccaca | |
| | | ggtgagcgggcgggacggcccttctcctccgggctgtaattagcgcttggtttaatga | |
| | | cggctcgtttcttttctgtggctgcgtgaaagccttaaagggctccgggagggcccttt | |
| | | gtgcgggggggagcggctcggggggtgcgtgcgtgtgtgtgtgcgtggggagcgcc | |
| | | gcgtgcggcccgcgctgcccggcggctgtgagcgctgcgggcgcggcgcggggctt | |
| | | tgtgcgctccgcgtgtgcgcgaggggagcgcggccgggggcggtgccccgcggtgc | |
| | | gggggggctgcgaggggaacaaaggctgcgtgcggggtgtgtgcgtgggggggtg | |
| | | agcagggggtgtgggcgcggcggtcgggctgtaacccccccctgcacccccctccc | |
| | | cgagttgctgagcacggcccggcttcgggtgcggggctccgtgcggggcgtggcgc | |
| | | ggggctcgccgtgccgggcggggggtggcggcaggtgggggtgccgggcggggcg | |
| | | gggccgcctcgggccggggagggctcgggggaggggcgcggcggccccggagcg | |
| | | ccggcggctgtcgaggcgcggcgagccgcagccattgccttttatggtaatcgtgcg | |
| | | agagggcgcagggacttcctttgtcccaaatctggcggagccgaaatctgggaggc | |
| | | gccgccgcaccccctctagcgggcgcgggcgaagcggtgcggcgccggcaggaag | |
| | | gaaatgggcggggagggccttcgtgcgtcgccgcgccgccgtccccttctccatctc | |
| | | cagcctcggggctgccgcagggggacggctgccttcgggggggacggggcagggc | |
| | | ggggttcggcttctggcgtgtgaccggcggctctagagcctctgctaaccatgttcat | |
| | | gccttcttctttttcctacaggggggatccgtttatctgcagaattcgcccttgacgtcg | |
| | | ccaccatggcgcttccggtgacagcactgctcctccccttggcgctgttgctccacgc | |
| | | agcaaggccggacatccagatgacccagagccccaccagcctgagcgccagcgtg | |
| | | ggcgacagagtgaccatcacctgcagagccagccagggcatcagcagctggctga | |
| | | cctggtaccagcagaagcccgagaaggcccccaagagcctgatctacgccgccag | |
| | | cagcctgcagagcggcgtgcccagcagattcagcggcagcggcagcggcaccgac | |
| | | ttcaccctgaccatcagcagcctgcagcccgaggacttcgccacctactactgccag | |
| | | cagtacgacagctaccccatcaccttcggccagggcaccagactggagatcaaggg | |
| | | cggcggcggcagcggcggcggcggcagcggcggcggcggcagccaggtgcagct | |
| | | gcagcagtggggcgccggcctgctgaagcccagcgagaccctgagcctgacctgcg | |
| | | ccgtgtacggcggcagcttcagcgcctactactggagctggatcagacagccccccg | |
| | | gcaagggcctggagtggatcggcgacatcaaccacggcggcggcaccaactacaa | |
| | | ccccagcctgaagagcagagtgaccatcagcgtggacaccagcaagaaccagttc | |
| | | agcctgaagctgaacagcgtgaccgccgccgacaccgccgtgtactactgcgccag | |
| | | cctgaccgcctactggggccagggcagcctggtgaccgtgagcgccgccgccttcgt | |
| | | gcccgtgttcctgcccgccaagcccaccaccacccccgcccccagaccccccacccc | |
| | | cgcccccaccatcgccagccagcccctgagcctgagacccgaggcctgcagacccg | |
| | | ccgccggcggcgccgtgcacaccagaggcctggacttcgcctgcgacatctacatct | |
| | | gggcccccctggccggcacctgcggcgtgctgctgctgagcctggtgatcaccctgt | |
| | | actgcaaccacagaaacagaaagagaggcagaaagaagctgctgtacatcttcaa | |
| | | gcagcccttcatgagacccgtgcagaccacccaggaggaggacggctgcagctgc | |
| | | agattccccgaggaggaggagggcggctgcgagctgagagtgaagttcagcagaa | |
| | | gcgccgacgcccccgcctaccagcagggccagaaccagctgtacaacgagctgaa | |
| | | cctgggcagaagagaggagtacgacgtgctggacaagagaagaggcagagaccc | |
| | | cgagatgggcggcaagcccagaagaaagaacccccaggagggcctgtacaacga | |
| | | gctgcagaaggacaagatggccgaggcctacagcgagatcggcatgaagggcga | |
| | | gagaagaagaggcaagggccacgacggcctgtaccagggcctgagcaccgccac | |
| | | caaggacacctacgacgccctgcacatgcaggccctgccccccagaggaagcgga | |
| | | gctactaacttcagcctgctgaagcaggctggagacgtggaggagaaccctggacc | |
| | | tatgtctcgctccgttgccttagctgtgctcgcgctactctctctttctggattagaggc | |
| | | tgtcatggcgccccgaaccctcttcctgggtggaggcggttcaggcggaggtggctc | |
| | | tggcggtggcggatcgatccagcgtactccaaagattcaggtttactcacgtcatcc | |
| | | agcagagaatggaaagtcaaatttcctgaattgctatgtgtctgggtttcatccatcc | |
| | | gacattgaagttgacttactgaagaatggagagagaattgaaaaagtggagcattc | |
| | | agacttgtctttcagcaaggactggtctttctatctcttgtactacactgaattcacccc | |
| | | cactgaaaaagatgagtatgcctgccgtgtgaaccatgtgactttgtcacagcccaa | |
| | | gatagttaagtgggatcgagacatgggtggtggtggttctggtggtggtggttctggc | |
| | | ggcggcggctccggtggtggtggatccggctcccactccttgaagtatttccacactt | |
| | | ccgtgtcccggcccggccgcggggagccccgcttcatctctgtgggctacgtggacg | |
| | | acacccagttcgtgcgcttcgacaacgacgccgcgagtccgaggatggtgccgcgg | |
| | | gcgccgtggatggagcaggaggggtcagagtattgggaccgggagacacggagcg | |
| | | ccagggacaccgcacagattttccgagtgaatctgcggacgctgcgcggctactac | |
| | | aatcagagcgaggccgggtctcacaccctgcagtggatgcatggctgcgagctggg | |
| | | gcccgacgggcgcttcctccgcgggtatgaacagttcgcctacgacggcaaggatta | |
| | | tctcaccctgaatgaggacctgcgctcctggaccgcggtggacacggcggctcagat | |
| | | ctccgagcaaaagtcaaatgatgcctctgaggcggagcaccagagagcctacctgg | |
| | | aagacacatgcgtggagtggctccacaaatacctggagaaggggaaggagacgct | |
| | | gcttcacctggagcccccaaagacacacgtgactcaccaccccatctctgaccatga | |
| | | ggccaccctgaggtgctgggccctgggcttctaccctgcggagatcacactgacctg | |
| | | gcagcaggatggggagggccatacccaggacacggagctcgtggagaccaggcct | |
| | | gcaggggatggaaccttccagaagtgggcagctgtggtggtgccttctggagagga | |
| | | gcagagatacacgtgccatgtgcagcatgaggggctacccgagcccgtcaccctga | |
| | | gatggaagccggcttcccagcccaccatccccatcgtgggcatcattgctggcctgg | |
| | | ttctccttggatctgtggtctctggagctgtggttgctgctgtgatatggaggaagaag | |
| | | agctcaggtggaaaaggagggagctactctaaggctgagtggagcgacagtgccc | |
| | | aggggtctgagtctcacagcttg | |
| HLA-E | Amino acid | MSRSVALAVLALLSLSGLEAVMAPRTLFLGGGGSGGG | 142 |
| trimer | sequence | GSGGGGSIQRTPKIQVYSRHPAENGKSNFLNCYVSGFH | |
| | | PSDIEVDLLKNGERIEKVEHSDLSFSKDWSFYLLYYTE | |
| | | FTPTEKDEYACRVNHVTLSQPKIVKWDRDMGGGGSG | |
| | | GGGSGGGGSGGGGSGSHSLKYFHTSVSRPGRGEPRFIS | |
| | | VGYVDDTQFVRFDNDAASPRMVPRAPWMEQEGSEY | |
| | | WDRETRSARDTAQIFRVNLRTLRGYYNQSEAGSHTLQ | |
| | | WMHGCELGPDGRFLRGYEQFAYDGKDYLTLNEDLRS | |
| | | WTAVDTAAQISEQKSNDASEAEHQRAYLEDTCVEWL | |
| | | HKYLEKGKETLLHLEPPKTHVTHHPISDHEATLRCWA | |
| | | LGFYPAEITLTWQQDGEGHTQDTELVETRPAGDGTFQ | |
| | | KWAAVVVPSGEEQRYTCHVQHEGLPEPVTLRWKPAS | |
| | | QPTIPIVGIIAGLVLLGSVVSGAVVAAVIWRKKSSGGK | |
| | | GGSYSKAEWSDSAQGSESHSL | |
| IL15/IL15 | Amino acid | MDWTWILFLVAAATRVHSGIHVFILGCFSAGLPKTEA | 143 |
| Rα fusion | sequence | NWVNVISDLKKIEDLIQSMHIDATLYTESDVHPSCKVT | |
| | | AMKCFLLELQVISLESGDASIHDTVENLIILANNSLSSN | |
| | | GNVTESGCKECEELEEKNIKEFLQSFVHIVQMFINTSSG | |
| | | GGSGGGGSGGGGSGGGGSGGGSLQITCPPPMSVEHAD | |
| | | IWVKSYSLYSRERYICNSGFKRKAGTSSLTECVLNKAT | |
| | | NVAHWTTPSLKCIRDPALVHQRPAPPSTVTTAGVTPQ | |
| | | PESLSPSGKEPAASSPSSNNTAATTAAIVPGSQLMPSKS | |
| | | PSTGTTEISSHESSHGTPSQTTAKNWELTASASHQPPG | |
| | | VYPQGHSDTTVAISTSTVLLCGLSAVSLLACYLKSRQT | |
| | | PPLASVEMEAMEALPVTWGTSSRDEDLENCSHHLGSG | |
| SERPINB9 | Amino acid | METLSNASGTFAIRLLKILCQDNPSHNVFCSPVSISSAL | 144 |
| | sequence | AMVLLGAKGNTATQMAQALSLNTEEDIHRAFQSLLTE | |
| | | VNKAGTQYLLRTANRLFGEKTCQFLSTFKESCLQFYH | |
| | | AELKELSFIRAAEESRKHINTWVSKKTEGKIEELLPGSS | |
| | | IDAETRLVLVNAIYFKGKWNEPFDETYTREMPFKINQE | |
| | | EQRPVQMMYQEATFKLAHVGEVRAQLLELPYARKEL | |
| | | SLLVLLPDDGVELSTVEKSLTFEKLTAWTKPDCMKST | |
| | | EVEVLLPKFKLQEDYDMESVLRHLGIVDAFQQGKADL | |
| | | SAMSAERDLCLSKFVHKSFVEVNEEGTEAAAASSCFV | |
| | | VAECCMESGPRFCADHPFLFFIRHNRANSILFCGRFSSP | |
| CISH Left | 1249-2248 | AAGCCCTAGTATCACCCTGCTGGGTGGGCTTCCCCT | 145 |
| HA | (1000) | AGGAGAAAGCTGCTACTCAGATTTTGGTCGGCTCAG | |
| | | TTCCCCCAACCCCAGCCATCCAAGAATATGGTGTTA | |
| | | CTGAACCTCTCAGTGCATGGGTTGGTGGGACCCTTT | |
| | | ATTGCGGAAAAGGGAAACTGAAGCCCAGTGAAGAG | |
| | | CAATCTTTGTACAAGATCGTAGAGCCTGGTAGGGCT | |
| | | GGCTTGCCAGGGTGGGAACTGGAGTGTGGATTAGG | |
| | | TCCCTGCCCTTGGTGGGAGCTCATACTGACAGGAGG | |
| | | TGAGACTTGCCAGTGCCTTTAGAGGCCACTGTCCAC | |
| | | CCCAGACCCTCCACTGGGAGAAGCAGAGGCCAGGA | |
| | | TAGGCTGACTTGTATGGGTAGGGGATTATTTCAGTC | |
| | | TCTAAGTCCTGGCCAGTGTGGACCTAGGTCCCTCTG | |
| | | AGAGACACTCCTATCCATGGGGAGGTAGCGTGTGA | |
| | | CCTCAGGCAGCCTACCATAATCCTGGGGCTTCTCTG | |
| | | GAGGACCCCACACAGATCTATCATAGGATAAATCT | |
| | | ACTTGTCTGTCGTAGATCTATCTCAACTAGGACCAT | |
| | | TCATCAGTGGGCCATTTAATCAGCCCACTTCAGCCA | |
| | | GGAGGGCACAAACATGAAGCTACTTTGCACTGAAT | |
| | | CCTATCAGTGTGGTGAGAACATTAGACATGCCTATA | |
| | | GGGAATGATCTGTGTTTTTGGTTGATATAAAATAGA | |
| | | GAGCAATTAATTACTGTTTAGTAAATGCAGGGCAGT | |
| | | GGAAAAGAATCTTTCAAAAGAGGATCCCCACATGG | |
| | | AAACAATCAAGGTGGCTTTTGTGATGACAAGTGGG | |
| | | GAACGAGATACTGGACTAACTGAGCCCATGGCTGG | |
| | | GTAGAGGGGAAGCTGAGGCTCATAGTGAGATGGGC | |
| | | CAGCCAGAGGTCATGAAACTAGTCTGGGGCATAGG | |
| | | GGGGTCTCTGGAGATGGGGGAAGCATGGGTCCTCC | |
| | | ACTCACTGCTCATCCACTGCCTTCtctTAGACCTCGTC | |
| | | CT | |
| CD64 gb | 3884-5373 | GTGACCGGCGGCTCTAGAGCCTCTGCTAACCATGTT | 146 |
| | (1490) | CATGCCTTCTTCTTTTTCCTACAGgggggatccgtttatctgcag | |
| | | aattcgcccttgacgtcgccaccatgtggttcttgacaactctgctcctttgggttccagt | |
| | | tgatgggcaagtggacaccacaaaggcagtgatcactttgcagcctccatgggtcag | |
| | | cgtgttccaagaggaaaccgtaaccttgcactgtgaggtgctccatctgcctgggagc | |
| | | agctctacacagtggtttctcaatggcacagccactcagacctcgacccccagctaca | |
| | | gaatcacctctgccagtgtcaatgacagtggtgaatacaggtgccagagaggtctctc | |
| | | agggcgaagtgaccccatacagctggaaatccacagaggctggctactactgcagg | |
| | | tctccagcagagtcttcacggaaggagaacctctggccttgaggtgtcatgcgtggaa | |
| | | ggataagctggtgtacaatgtgctttactatcgaaatggcaaagcctttaagtttttccac | |
| | | tggaattctaacctcaccattctgaaaaccaacataagtcacaatggcacctaccattgc | |
| | | tcaggcatgggaaagcatcgctacacatcagcaggaatatctgtcactgtgaaagag | |
| | | ctatttccagctccagtgctgaatgcatctgtgacatccccactcctggaggggaatct | |
| | | ggtcaccctgagctgtgaaacaaagttgctcttgcagaggcctggtttgcagctttactt | |
| | | ctccttctacatgggcagcaagaccctgcgaggcaggaacacatcctctgaatacca | |
| | | aatactaactgctagaagagaagactctgggttatactggtgcgaggctgccacagag | |
| | | gatggaaatgtccttaagcgcagccctgagttggagcttcaagtgcttggcctccagtt | |
| | | accaactcctgtctggtttcatgtccttttctatctggcagtgggaataatgtttttagtgaa | |
| | | cactgttctctgggtgacaatacgtaaagaactgaaaagaaagaaaaagtgggattta | |
| | | gaaatctctttggattctggtcatgagaagaaggtaatttccagccttcaagaagacag | |
| | | acatttagaagaagagctgaaatgtcaggaacaaaaagaagaacagctgcaggaag | |
| | | gggtgcaccggaaggagccccagggggccacgtagCCGCTGATCAGC | |
| | | CTCGACTGTGCCTTCTAGTTGCCAGCCATCTGTTGTT | |
| | | TGCCCCTCCCCCGTGCCTTCCTTGACCCTGGAAGGT | |
| | | GCCACTCCCACTGTCCTTTCCTAATAAAATGAGGAA | |
| | | ATTGCATCGCATTGTCTGAGTAGGTGTCATTCTATTC | |
| | | TGGGGGGTGGGGTGGGGCAGGACAGCAAGGGGGA | |
| | | GGATTGGGAAGACAATAGCAGGCATGCTGGGGATG | |
| | | CGGTGGGCTCTATGGGTCGACGCGGACTGGGCAG | |
| CD64 | 3988-5112 | Atgtggttcttgacaactctgctcctttgggttccagttgatgggcaagtggacaccac | 147 |
| FCGR1A | (1125) | aaaggcagtgatcactttgcagcctccatgggtcagcgtgttccaagaggaaaccgta | |
| | | accttgcactgtgaggtgctccatctgcctgggagcagctctacacagtggtttctcaat | |
| | | ggcacagccactcagacctcgacccccagctacagaatcacctctgccagtgtcaat | |
| | | gacagtggtgaatacaggtgccagagaggtctctcagggcgaagtgaccccataca | |
| | | gctggaaatccacagaggctggctactactgcaggtctccagcagagtcttcacgga | |
| | | aggagaacctctggccttgaggtgtcatgcgtggaaggataagctggtgtacaatgtg | |
| | | ctttactatcgaaatggcaaagcctttaagtttttccactggaattctaacctcaccattct | |
| | | gaaaaccaacataagtcacaatggcacctaccattgctcaggcatgggaaagcatcg | |
| | | ctacacatcagcaggaatatctgtcactgtgaaagagctatttccagctccagtgctga | |
| | | atgcatctgtgacatccccactcctggaggggaatctggtcaccctgagctgtgaaac | |
| | | aaagttgctcttgcagaggcctggtttgcagctttacttctccttctacatgggcagcaa | |
| | | gaccctgcgaggcaggaacacatcctctgaataccaaatactaactgctagaagaga | |
| | | agactctgggttatactggtgcgaggctgccacagaggatggaaatgtccttaagcgc | |
| | | agccctgagttggagcttcaagtgcttggcctccagttaccaactcctgtctggtttcat | |
| | | gtccttttctatctggcagtgggaataatgtttttagtgaacactgttctctgggtgacaat | |
| | | acgtaaagaactgaaaagaaagaaaaagtgggatttagaaatctctttggattctggtc | |
| | | atgagaagaaggtaatttccagccttcaagaagacagacatttagaagaagagctgaa | |
| | | atgtcaggaacaaaaagaagaacagctgcaggaaggggtgcaccggaaggagcc | |
| | | ccagggggccacgtag | |
| CISH | 5361-6360 | GCGGACTGGGCAGCGGCCCCTGTGGGCCCCGTCCCT | 148 |
| Right HA | (1000) | GGAACTGCCCAAGCCAGTCATGCAGCCCTTGCCTGC | |
| | | TGGGGCCTTCCTCGAGGAGGTGGCAGAGGGTACCC | |
| | | CAGCCCAGACAGAGAGTGAGCCAAAGGTGCTGGAC | |
| | | CCAGAGGAGGATCTGCTGTGCATAGCCAAGACCTTC | |
| | | TCCTACCTTCGGGAATCTGGTGAGTCTGAGGGGGGA | |
| | | GGCAGGCCTTTTCCTGAGTAGTCTGGTGGGAGAAtC | |
| | | TTAGTTCTGCCTTTGAGTCTGATTTGGGAGGCATAG | |
| | | CCCGGCCCAGGAGAGTCTGATGGGAGAGGCACAGC | |
| | | CCTCTCTAGGGAAGCGTGAAGCTGGAGGCACTGCCT | |
| | | TGACTTGCACTGGATTGGTTACCCCAGCTAATTTCC | |
| | | ACTATGTCCCTTGGCCCCCTCTGCACTTGCCTAGGCT | |
| | | GGTATTGGGGTTCCATTACGGCCAGCGAGGCCCGAC | |
| | | AACACCTGCAGAAGATGCCAGAAGGCACGTTCTTA | |
| | | GTACGTGACAGCACGCACCCCAGCTACCTGTTCACG | |
| | | CTGTCAGTGAAAACCACTCGTGGCCCCACCAATGTA | |
| | | CGCATTGAGTATGCCGACTCCAGCTTCCGTCTGGAC | |
| | | TCCAACTGCTTGTCCAGGCCACGCATCCTGGCCTTT | |
| | | CCGGATGTGGTCAGCCTTGTGCAGCACTATGTGGCC | |
| | | TCCTGCACTGCTGATACCCGAAGCGACAGCCCCGAT | |
| | | CCTGCTCCCACCCCGGCCCTGCCTATGCCTAAGGAG | |
| | | GATGCGCCTAGTGACCCAGCACTGCCTGCTCCTCCA | |
| | | CCAGCCACTGCTGTACACCTAAAACTGGTGCAGCCC | |
| | | TTTGTACGCAGAAGCAGTGCCCGCAGCCTGCAACAC | |
| | | CTGTGCCGCCTTGTCATCAACCGTCTGGTGGCCGAC | |
| | | GTGGACTGCCTGCCACTGCCCCGGCGCATGGCCGAC | |
| | | TACCTCCGACAGTACCCCTTCCAGCTCTGACTGTAC | |
| | | GGGGCAATCTGCCCACCCTCACCCAGTCGCA | |
| Entire | (9703) | AAACAGCATTCCAGGTATTAGAAGAATATCCTGATT | 149 |
| Plasmid | | CAGGTGAAAATATTGTTGATGCGCTGGCAGTGTTCC | |
| | | TGCGCCGGTTGCATTCGATTCCTGTTTGTAATTGTCC | |
| | | TTTTAACAGCGATCGCGTATTTCGTCTCGCTCAGGC | |
| | | GCAATCACGAATGAATAACGGTTTGGTTGATGCGA | |
| | | GTGATTTTGATGACGAGCGTAATGGCTGGCCTGTTG | |
| | | AACAAGTCTGGAAAGAAATGCATAAACTTTTGCCAT | |
| | | TCTCACCGGATTCAGTCGTCACTCATGGTGATTTCTC | |
| | | ACTTGATAACCTTATTTTTGACGAGGGGAAATTAAT | |
| | | AGGTTGTATTGATGTTGGACGAGTCGGAATCGCAGA | |
| | | CCGATACCAGGATCTTGCCATCCTATGGAACTGCCT | |
| | | CGGTGAGTTTTCTCCTTCATTACAGAAACGGCTTTTT | |
| | | CAAAAATATGGTATTGATAATCCTGATATGAATAAA | |
| | | TTGCAGTTTCATTTGATGCTCGATGAGTTTTTCTAAT | |
| | | CTCATGACCAAAATCCCTTAACGTGAGTTTTCGTTC | |
| | | CACTGAGCGTCAGACCCCGTAGAAAAGATCAAAGG | |
| | | ATCTTCTTGAGATCCTTTTTTTCTGCGCGTAATCTGC | |
| | | TGCTTGCAAACAAAAAAACCACCGCTACCAGCGGT | |
| | | GGTTTGTTTGCCGGATCAAGAGCTACCAACTCTTTT | |
| | | TCCGAAGGTAACTGGCTTCAGCAGAGCGCAGATAC | |
| | | CAAATACTGTCCTTCTAGTGTAGCCGTAGTTAGGCC | |
| | | ACCACTTCAAGAACTCTGTAGCACCGCCTACATACC | |
| | | TCGCTCTGCTAATCCTGTTACCAGTGGCTGCTGCCA | |
| | | GTGGCGATAAGTCGTGTCTTACCGGGTTGGACTCAA | |
| | | GACGATAGTTACCGGATAAGGCGCAGCGGTCGGGC | |
| | | TGAACGGGGGGTTCGTGCACACAGCCCAGCTTGGA | |
| | | GCGAACGACCTACACCGAACTGAGATACCTACAGC | |
| | | GTGAGCTATGAGAAAGCGCCACGCTTCCCGAAGGG | |
| | | AGAAAGGCGGACAGGTATCCGGTAAGCGGCAGGGT | |
| | | CGGAACAGGAGAGCGCACGAGGGAGCTTCCAGGGG | |
| | | GAAACGCCTGGTATCTTTATAGTCCTGTCGGGTTTC | |
| | | GCCACCTCTGACTTGAGCGTCGATTTTTGTGATGCT | |
| | | CGTCAGGGGGGCGGAGCCTATGGAAAAACGCCAGC | |
| | | AACGCGGCCTTTTTACGGTTCCTGGCCTTTTGCTGGC | |
| | | CTTTTGCTCACATGTGCGGCCGCACGCGTAAGCCCT | |
| | | AGTATCACCCTGCTGGGTGGGCTTCCCCTAGGAGAA | |
| | | AGCTGCTACTCAGATTTTGGTCGGCTCAGTTCCCCC | |
| | | AACCCCAGCCATCCAAGAATATGGTGTTACTGAACC | |
| | | TCTCAGTGCATGGGTTGGTGGGACCCTTTATTGCGG | |
| | | AAAAGGGAAACTGAAGCCCAGTGAAGAGCAATCTT | |
| | | TGTACAAGATCGTAGAGCCTGGTAGGGCTGGCTTGC | |
| | | CAGGGTGGGAACTGGAGTGTGGATTAGGTCCCTGC | |
| | | CCTTGGTGGGAGCTCATACTGACAGGAGGTGAGAC | |
| | | TTGCCAGTGCCTTTAGAGGCCACTGTCCACCCCAGA | |
| | | CCCTCCACTGGGAGAAGCAGAGGCCAGGATAGGCT | |
| | | GACTTGTATGGGTAGGGGATTATTTCAGTCTCTAAG | |
| | | TCCTGGCCAGTGTGGACCTAGGTCCCTCTGAGAGAC | |
| | | ACTCCTATCCATGGGGAGGTAGCGTGTGACCTCAGG | |
| | | CAGCCTACCATAATCCTGGGGCTTCTCTGGAGGACC | |
| | | CCACACAGATCTATCATAGGATAAATCTACTTGTCT | |
| | | GTCGTAGATCTATCTCAACTAGGACCATTCATCAGT | |
| | | GGGCCATTTAATCAGCCCACTTCAGCCAGGAGGGC | |
| | | ACAAACATGAAGCTACTTTGCACTGAATCCTATCAG | |
| | | TGTGGTGAGAACATTAGACATGCCTATAGGGAATG | |
| | | ATCTGTGTTTTTGGTTGATATAAAATAGAGAGCAAT | |
| | | TAATTACTGTTTAGTAAATGCAGGGCAGTGGAAAA | |
| | | GAATCTTTCAAAAGAGGATCCCCACATGGAAACAA | |
| | | TCAAGGTGGCTTTTGTGATGACAAGTGGGGAACGA | |
| | | GATACTGGACTAACTGAGCCCATGGCTGGGTAGAG | |
| | | GGGAAGCTGAGGCTCATAGTGAGATGGGCCAGCCA | |
| | | GAGGTCATGAAACTAGTCTGGGGCATAGGGGGGTC | |
| | | TCTGGAGATGGGGGAAGCATGGGTCCTCCACTCACT | |
| | | GCTCATCCACTGCCTTCtctTAGACCTCGTCCTaagcttgtg | |
| | | gacgatatcgaattcgcacgacattgattattgactagtTATTAATAGTAAT | |
| | | CAATTACGGGGTCATTAGTTCATAGCCCATATATGG | |
| | | AGTTCCGCGTTACATAACTTACGGTAAATGGCCCGC | |
| | | CTGGCTGACCGCCCAACGACCCCCGCCCATTGACGT | |
| | | CAATAATGACGTATGTTCCCATAGTAACGCCAATAG | |
| | | GGACTTTCCATTGACGTCAATGGGTGGACTATTTAC | |
| | | GGTAAACTGCCCACTTGGCAGTACATCAAGTGTATC | |
| | | ATATGCCAAGTACGCCCCCTATTGACGTCAATGACG | |
| | | GTAAATGGCCCGCCTGGCATTATGCCCAGTACATGA | |
| | | CCTTATGGGACTTTCCTACTTGGCAGTACATCTACG | |
| | | TATTAGTCATCGCTATTACCATGGGTCGAGGTGAGC | |
| | | CCCACGTTCTGCTTCACTCTCCCCATCTCCCCCCCCT | |
| | | CCCCACCCCCAATTTTGTATTTATTTATTTTTTAATT | |
| | | ATTTTGTGCAGCGATGGGGGCGGGGGGGGGGGGGG | |
| | | CGCGCGCCAGGCGGGGCGGGGCGGGGCGAGGGGC | |
| | | GGGGCGGGGCGAGGCGGAGAGGTGCGGCGGCAGC | |
| | | CAATCAGAGCGGCGCGCTCCGAAAGTTTCCTTTTAT | |
| | | GGCGAGGCGGCGGCGGCGGCGGCCCTATAAAAAGC | |
| | | GAAGCGCGCGGCGGGCGGGAGTCGCTGCGTTGCCT | |
| | | TCGCCCCGTGCCCCGCTCCGCGCCGCCTCGCGCCGC | |
| | | CCGCCCCGGCTCTGACTGACCGCGTTACTCCCACAG | |
| | | GTGAGCGGGCGGGACGGCCCTTCTCCTCCGGGCTGT | |
| | | AATTAGCGCTTGGTTTAATGACGGCTCGTTTCTTTTC | |
| | | TGTGGCTGCGTGAAAGCCTTAAAGGGCTCCGGGAG | |
| | | GGCCCTTTGTGCGGGGGGGAGCGGCTCGGGGGGTG | |
| | | CGTGCGTGTGTGTGTGCGTGGGGAGCGCCGCGTGCG | |
| | | GCCCGCGCTGCCCGGCGGCTGTGAGCGCTGCGGGC | |
| | | GCGGCGCGGGGCTTTGTGCGCTCCGCGTGTGCGCGA | |
| | | GGGGAGCGCGGCCGGGGGCGGTGCCCCGCGGTGCG | |
| | | GGGGGGCTGCGAGGGGAACAAAGGCTGCGTGCGGG | |
| | | GTGTGTGCGTGGGGGGGTGAGCAGGGGGTGTGGGC | |
| | | GCGGCGGTCGGGCTGTAACCCCCCCCTGCACCCCCC | |
| | | TCCCCGAGTTGCTGAGCACGGCCCGGCTTCGGGTGC | |
| | | GGGGCTCCGTGCGGGGCGTGGCGCGGGGCTCGCCG | |
| | | TGCCGGGCGGGGGGTGGCGGCAGGTGGGGGTGCCG | |
| | | GGCGGGGCGGGGCCGCCTCGGGCCGGGGAGGGCTC | |
| | | GGGGGAGGGGCGCGGCGGCCCCGGAGCGCCGGCGG | |
| | | CTGTCGAGGCGCGGCGAGCCGCAGCCATTGCCTTTT | |
| | | ATGGTAATCGTGCGAGAGGGCGCAGGGACTTCCTTT | |
| | | GTCCCAAATCTGGCGGAGCCGAAATCTGGGAGGCG | |
| | | CCGCCGCACCCCCTCTAGCGGGCGCGGGCGAAGCG | |
| | | GTGCGGCGCCGGCAGGAAGGAAATGGGCGGGGAG | |
| | | GGCCTTCGTGCGTCGCCGCGCCGCCGTCCCCTTCTC | |
| | | CATCTCCAGCCTCGGGGCTGCCGCAGGGGGACGGC | |
| | | TGCCTTCGGGGGGGACGGGGCAGGGCGGGGTTCGG | |
| | | CTTCTGGCGTGTGACCGGCGGCTCTAGAGCCTCTGC | |
| | | TAACCATGTTCATGCCTTCTTCTTTTTCCTACAGgggg | |
| | | gatccgtttatctgcagaattcgcccttgacgtcgccaccatgtggttcttgacaactctg | |
| | | ctcctttgggttccagttgatgggcaagtggacaccacaaaggcagtgatcactttgca | |
| | | gcctccatgggtcagcgtgttccaagaggaaaccgtaaccttgcactgtgaggtgctc | |
| | | catctgcctgggagcagctctacacagtggtttctcaatggcacagccactcagacct | |
| | | cgacccccagctacagaatcacctctgccagtgtcaatgacagtggtgaatacaggtg | |
| | | ccagagaggtctctcagggcgaagtgaccccatacagctggaaatccacagaggct | |
| | | ggctactactgcaggtctccagcagagtcttcacggaaggagaacctctggccttgag | |
| | | gtgtcatgcgtggaaggataagctggtgtacaatgtgctttactatcgaaatggcaaag | |
| | | cctttaagtttttccactggaattctaacctcaccattctgaaaaccaacataagtcacaat | |
| | | ggcacctaccattgctcaggcatgggaaagcatcgctacacatcagcaggaatatctg | |
| | | tcactgtgaaagagctatttccagctccagtgctgaatgcatctgtgacatccccactcc | |
| | | tggaggggaatctggtcaccctgagctgtgaaacaaagttgctcttgcagaggcctgg | |
| | | tttgcagctttacttctccttctacatgggcagcaagaccctgcgaggcaggaacacat | |
| | | cctctgaataccaaatactaactgctagaagagaagactctgggttatactggtgcgag | |
| | | gctgccacagaggatggaaatgtccttaagcgcagccctgagttggagcttcaagtg | |
| | | cttggcctccagttaccaactcctgtctggtttcatgtccttttctatctggcagtgggaat | |
| | | aatgtttttagtgaacactgttctctgggtgacaatacgtaaagaactgaaaagaaagaa | |
| | | aaagtgggatttagaaatctctttggattctggtcatgagaagaaggtaatttccagcctt | |
| | | caagaagacagacatttagaagaagagctgaaatgtcaggaacaaaaagaagaaca | |
| | | gctgcaggaaggggtgcaccggaaggagccccagggggccacgtagCCGCT | |
| | | GATCAGCCTCGACTGTGCCTTCTAGTTGCCAGCCAT | |
| | | CTGTTGTTTGCCCCTCCCCCGTGCCTTCCTTGACCCT | |
| | | GGAAGGTGCCACTCCCACTGTCCTTTCCTAATAAAA | |
| | | TGAGGAAATTGCATCGCATTGTCTGAGTAGGTGTCA | |
| | | TTCTATTCTGGGGGGTGGGGTGGGGCAGGACAGCA | |
| | | AGGGGGAGGATTGGGAAGACAATAGCAGGCATGCT | |
| | | GGGGATGCGGTGGGCTCTATGGGTCGACGCGGACT | |
| | | GGGCAGCGGCCCCTGTGGGCCCCGTCCCTGGAACTG | |
| | | CCCAAGCCAGTCATGCAGCCCTTGCCTGCTGGGGCC | |
| | | TTCCTCGAGGAGGTGGCAGAGGGTACCCCAGCCCA | |
| | | GACAGAGAGTGAGCCAAAGGTGCTGGACCCAGAGG | |
| | | AGGATCTGCTGTGCATAGCCAAGACCTTCTCCTACC | |
| | | TTCGGGAATCTGGTGAGTCTGAGGGGGGAGGCAGG | |
| | | CCTTTTCCTGAGTAGTCTGGTGGGAGAAtCTTAGTTC | |
| | | TGCCTTTGAGTCTGATTTGGGAGGCATAGCCCGGCC | |
| | | CAGGAGAGTCTGATGGGAGAGGCACAGCCCTCTCT | |
| | | AGGGAAGCGTGAAGCTGGAGGCACTGCCTTGACTT | |
| | | GCACTGGATTGGTTACCCCAGCTAATTTCCACTATG | |
| | | TCCCTTGGCCCCCTCTGCACTTGCCTAGGCTGGTATT | |
| | | GGGGTTCCATTACGGCCAGCGAGGCCCGACAACAC | |
| | | CTGCAGAAGATGCCAGAAGGCACGTTCTTAGTACGT | |
| | | GACAGCACGCACCCCAGCTACCTGTTCACGCTGTCA | |
| | | GTGAAAACCACTCGTGGCCCCACCAATGTACGCATT | |
| | | GAGTATGCCGACTCCAGCTTCCGTCTGGACTCCAAC | |
| | | TGCTTGTCCAGGCCACGCATCCTGGCCTTTCCGGAT | |
| | | GTGGTCAGCCTTGTGCAGCACTATGTGGCCTCCTGC | |
| | | ACTGCTGATACCCGAAGCGACAGCCCCGATCCTGCT | |
| | | CCCACCCCGGCCCTGCCTATGCCTAAGGAGGATGCG | |
| | | CCTAGTGACCCAGCACTGCCTGCTCCTCCACCAGCC | |
| | | ACTGCTGTACACCTAAAACTGGTGCAGCCCTTTGTA | |
| | | CGCAGAAGCAGTGCCCGCAGCCTGCAACACCTGTG | |
| | | CCGCCTTGTCATCAACCGTCTGGTGGCCGACGTGGA | |
| | | CTGCCTGCCACTGCCCCGGCGCATGGCCGACTACCT | |
| | | CCGACAGTACCCCTTCCAGCTCTGACTGTACGGGGC | |
| | | AATCTGCCCACCCTCACCCAGTCGCAGGTAACCACG | |
| | | TGCGGACCGggctccggtgcccgtcagtgggcagagcgcacatcgcccac | |
| | | agtccccgagaagttggggggaggggtcggcaattgaaccggtgcctagagaaggt | |
| | | ggcgcggggtaaactgggaaagtgatgtcgtgtactggctccgcctttttcccgaggg | |
| | | tgggggagaaccgtatataagtgcagtagtcgccgtgaacgttctttttcgcaacgggt | |
| | | ttgccgccagaacacaggtaagtgccgtgtgtggttcccgcgggcctggcctctttac | |
| | | gggttatggcccttgcgtgccttgaattacttccactggctgcagtacgtgattcttgatc | |
| | | ccgagcttcgggttggaagtgggtgggagagttcgaggccttgcgcttaaggagccc | |
| | | cttcgcctcgtgcttgagttgaggcctggcctgggcgctggggccgccgcgtgcgaa | |
| | | tctggtggcaccttcgcgcctgtctcgctgctttcgataagtctctagccatttaaaattttt | |
| | | gatgacctgctgcgacgctttttttctggcaagatagtcttgtaaatgcgggccaagatc | |
| | | tgcacactggtatttcggtttttggggccgcgggcggcgacggggcccgtgcgtccc | |
| | | agcgcacatgttcggcgaggcggggcctgcgagcgcggccaccgagaatcggac | |
| | | gggggtagtctcaagctggccggcctgctctggtgcctggcctcgcgccgccgtgta | |
| | | tcgccccgccctgggcggcaaggctggcccggtcggcaccagttgcgtgagcgga | |
| | | aagatggccgcttcccggccctgctgcagggagctcaaaatggaggacgcggcgct | |
| | | cgggagagcgggcgggtgagtcacccacacaaaggaaaagggcctttccgtcctc | |
| | | agccgtcgcttcatgtgactccacggagtaccgggcgccgtccaggcacctcgatta | |
| | | gttctcgagcttttggagtacgtcgtctttaggttggggggaggggttttatgcgatgga | |
| | | gtttccccacactgagtgggtggagactgaagttaggccagcttggcacttgatgtaat | |
| | | tctccttggaatttgccctttttgagtttggatcttggttcattctcaagcctcagacagtgg | |
| | | ttcaaagtttttttcttccatttcaggtgtcgtgacttgacgtcgccaccatgaggatatttg | |
| | | ctgtctttatattcatgacctactggcatttgctgaacgcatttactgtcacggttcccaag | |
| | | gacctatatgtggtagagtatggtagcaatatgacaattgaatgcaaattcccagtaga | |
| | | aaaacaattagacctggctgcactaattgtctattgggaaatggaggataagaacattat | |
| | | tcaatttgtgcatggagaggaagacctgaaggttcagcatagtagctacagacagag | |
| | | ggcccggctgttgaaggaccagctctccctgggaaatgctgcacttcagatcacagat | |
| | | gtgaaattgcaggatgcaggggtgtaccgctgcatgatcagctatggtggtgccgact | |
| | | acaagcgaattactgtgaaagtcaatgccccatacaacaaaatcaaccaaagaattttg | |
| | | gttgtggatccagtcacctctgaacatgaactgacatgtcaggctgagggctacccca | |
| | | aggccgaagtcatctggacaagcagtgaccatcaagtcctgagtggtaagaccacca | |
| | | ccaccaattccaagagagaggagaaacttttcaatgtgaccagcacactgagaatcaa | |
| | | cacaacaactaatgagattttctactgcacttttaggagattagatcctgaggaaaacca | |
| | | tacagctgaattggtcatcccagaactacctctggcacatcctccaaatgaaaggactc | |
| | | acttggtaattctgggagccatcttattatgccttggtgtagcactgacattcatcttccgtt | |
| | | taagaaaagggagaatgatggatgtgaaaaaatgtggcatccaagatacaaactcaa | |
| | | agaagcaaagtgatacacatttggaggagacgtaaCCGCTGATCAGCCT | |
| | | CGAAACTTGTTTATTGCAGCTTATAATGGTTACAAA | |
| | | TAAAGCAATAGCATCACAAATTTCACAAATAAAGC | |
| | | ATTTTTTTCACTGCATTCTAGTTGTGGTTTGTCCAAA | |
| | | CTCATCAATGTATCTTAGGCGCCTGATGCGGTATTT | |
| | | TCTCCTTACGCATCTGTGCGGTATTTCACACCGCAT | |
| | | ACAGTACTGTCAAAGCAACCATAGTACGCGCCCTGT | |
| | | AGCGGCGCATTAAGCGCGGCGGGTGTGGTGGTTAC | |
| | | GCGCAGCGTGACCGCTACACTTGCCAGCGCCCTAGC | |
| | | GCCCGCTCCTTTCGCTTTCTTCCCTTCCTTTCTCGCC | |
| | | ACGTTCGCCGGCTTTCCCCGTCAAGCTCTAAATCGG | |
| | | GGGCTCCCTTTAGGGTTCCGATTTAGTGCTTTACGG | |
| | | CACCTCGACCCCAAAAAACTTGATTTGGGTGATGGT | |
| | | TCACGTAGTGGGCCATCGCCCTGATAGACGGTTTTT | |
| | | CGCCCTTTGACGTTGGAGTCCACGTTCTTTAATAGT | |
| | | GGACTCTTGTTCCAAACTGGAACAACACTCAACCCT | |
| | | ATCTCGGGCTATTCTTTTGATTTATAAGGGATTTTGC | |
| | | CGATTTCGGCCTATTGGTTAAAAAATGAGCTGATTT | |
| | | AACAAAAATTTAACGCGAATTTTAACAAAATATTAA | |
| | | CGTTTACAATTTTATGGTGCACTCTCAGTACAATCT | |
| | | GCTCTGATGCCGCATAGTTAAGCCAGCCCCGACACC | |
| | | CGCCAACACCCGCTGACGCGCCCTGACGGGCTTGTC | |
| | | TGCTCCCGGCATCCGCTTACAGACAAGCTGTGACCG | |
| | | TCTCCGGGAGCTGCATGTGTCAGAGGTTTTCACCGT | |
| | | CATCACCGAAACGCGCGAGACGAAAGGGCCTCGTG | |
| | | ATACGCCTATTTTTATAGGTTAATGTCATGAACAAT | |
| | | AAAACTGTCTGCTTACATAAACAGTAATACAAGGG | |
| | | GTGTTATGAGCCATATTCAACGGGAAACGTCGAGG | |
| | | CCGCGATTAAATTCCAACATGGATGCTGATTTATAT | |
| | | GGGTATAAATGGGCTCGCGATAATGTCGGGCAATC | |
| | | AGGTGCGACAATCTATCGCTTGTATGGGAAGCCCGA | |
| | | TGCGCCAGAGTTGTTTCTGAAACATGGCAAAGGTAG | |
| | | CGTTGCCAATGATGTTACAGATGAGATGGTCAGACT | |
| | | AAACTGGCTGACGGAATTTATGCCTCTTCCGACCAT | |
| | | CAAGCATTTTATCCGTACTCCTGATGATGCATGGTT | |
| | | ACTCACCACTGCGATCCCCGGAA | |
| CD64 | Amino acid | MWFLTTLLLWVPVDGQVDTTKAVITLQPPWVSVFQE | 150 |
| | sequence | ETVTLHCEVLHLPGSSSTQWFLNGTATQTSTPSYRITS | |
| | | ASVNDSGEYRCQRGLSGRSDPIQLEIHRGWLLLQVSSR | |
| | | VFTEGEPLALRCHAWKDKLVYNVLYYRNGKAFKFFH | |
| | | WNSNLTILKTNISHNGTYHCSGMGKHRYTSAGISVTV | |
| | | KELFPAPVLNASVTSPLLEGNLVTLSCETKLLLQRPGL | |
| | | QLYFSFYMGSKTLRGRNTSSEYQILTARREDSGLYWC | |
| | | EAATEDGNVLKRSPELELQVLGLQLPTPVWFHVLFYL | |
| | | AVGIMFLVNTVLWVTIRKELKRKKKWDLEISLDSGHE | |
| | | KKVISSLQEDRHLEEELKCQEQKEEQLQEGVHRKEPQ | |
| | | GAT | |
| TGFBRII | gRNA target in | TGCTGGCGATACGCGTCCAC | 151, |
| Ex1-T2 | TGFBRII, spacer | | 211 |
| | sequence | | |
| GPC3 | Nucleic Acid | ATGGCGCTTCCGGTGACAGCACTGCTCCTCCCCTTG | 152 |
| CAR | Sequence | GCGCTGTTGCTCCACGCAGCAAGGCCGCAAGTGCA | |
| | | GCTCGTGCAAAGCGGCGGCGGACTGGTGCAACCCG | |
| | | GCGGATCTCTGAGACTGAGCTGCGCCGCCAGCTACT | |
| | | TCGACTTCGACAGCTACGAGATGAGCTGGGTGAGG | |
| | | CAAGCCCCCGGAAAAGGACTGGAGTGGATCGGCAG | |
| | | CATCTACCACAGCGGCTCCACCTACTACAACCCCTC | |
| | | TCTGAAATCTAGAGTGACCATCTCTAGAGACAACTC | |
| | | CAAGAACACACTGTACCTCCAGATGAACACACTGA | |
| | | GAGCCGAGGACACCGCCACCTATTACTGCGCTAGA | |
| | | GTGAACATGGACAGATTCGACTACTGGGGCCAAGG | |
| | | CACACTGGTGACAGTGAGCTCCAGTGCTGCTGCCTT | |
| | | TGTCCCGGTATTTCTCCCAGCCAAACCGACCACGAC | |
| | | TCCCGCCCCGCGCCCTCCGACACCCGCTCCCACCAT | |
| | | CGCCTCTCAACCTCTTAGTCTTCGCCCCGAGGCATG | |
| | | CCGACCCGCCGCCGGGGGTGCTGTTCATACGAGGG | |
| | | GCTTGGACTTCGCTTGTGATATTTACATTTGGGCTCC | |
| | | GTTGGCGGGTACGTGCGGCGTCCTTTTGTTGTCACT | |
| | | CGTTATTACTTTGTATTGTAATCACAGGAATCGCTC | |
| | | AAAGCGGAGTAGGTTGTTGCATTCCGATTACATGAA | |
| | | TATGACTCCTCGCCGGCCTGGGCCGACAAGAAAAC | |
| | | ATTACCAACCCTATGCCCCCCCACGAGACTTCGCTG | |
| | | CGTACAGGTCCCGAGTGAAGTTTTCCCGAAGCGCAG | |
| | | ACGCTCCGGCATATCAGCAAGGACAGAATCAGCTG | |
| | | TATAACGAACTGAATTTGGGACGCCGCGAGGAGTA | |
| | | TGACGTGCTTGATAAACGCCGGGGGAGAGACCCGG | |
| | | AAATGGGGGGTAAACCCCGAAGAAAGAATCCCCAA | |
| | | GAAGGACTCTACAATGAACTCCAGAAGGATAAGAT | |
| | | GGCGGAGGCCTACTCAGAAATAGGTATGAAGGGCG | |
| | | AACGACGACGGGGAAAAGGTCACGATGGCCTCTAC | |
| | | CAAGGGTTGAGTACGGCAACCAAAGATACGTACGA | |
| | | TGCACTGCATATGCAGGCCCTGCCTCCCAGA | |
| HN3_single_ | Nucleic Acid | CAAGTGCAGCTCGTGCAAAGCGGCGGCGGACTGGT | 153 |
| domain | Sequence | GCAACCCGGCGGATCTCTGAGACTGAGCTGCGCCG | |
| | | CCAGCTACTTCGACTTCGACAGCTACGAGATGAGCT | |
| | | GGGTGAGGCAAGCCCCCGGAAAAGGACTGGAGTGG | |
| | | ATCGGCAGCATCTACCACAGCGGCTCCACCTACTAC | |
| | | AACCCCTCTCTGAAATCTAGAGTGACCATCTCTAGA | |
| | | GACAACTCCAAGAACACACTGTACCTCCAGATGAA | |
| | | CACACTGAGAGCCGAGGACACCGCCACCTATTACT | |
| | | GCGCTAGAGTGAACATGGACAGATTCGACTACTGG | |
| | | GGCCAAGGCACACTGGTGACAGTGAGCTCC | |
| CD28 | Nucleic Acid | TCAAAGCGGAGTAGGTTGTTGCATTCCGATTACATG | 154 |
| | Sequence | AATATGACTCCTCGCCGGCCTGGGCCGACAAGAAA | |
| | | ACATTACCAACCCTATGCCCCCCCACGAGACTTCGC | |
| | | TGCGTACAGGTCC | |
| Cd3z | Nucleic Acid | CGAGTGAAGTTTTCCCGAAGCGCAGACGCTCCGGC | 155 |
| | Sequence | ATATCAGCAAGGACAGAATCAGCTGTATAACGAAC | |
| | | TGAATTTGGGACGCCGCGAGGAGTATGACGTGCTTG | |
| | | ATAAACGCCGGGGGAGAGACCCGGAAATGGGGGGT | |
| | | AAACCCCGAAGAAAGAATCCCCAAGAAGGACTCTA | |
| | | CAATGAACTCCAGAAGGATAAGATGGCGGAGGCCT | |
| | | ACTCAGAAATAGGTATGAAGGGCGAACGACGACGG | |
| | | GGAAAAGGTCACGATGGCCTCTACCAAGGGTTGAG | |
| | | TACGGCAACCAAAGATACGTACGATGCACTGCATA | |
| | | TGCAGGCCCTGCCTCCCAGA | |
| Entire | Nucleic Acid | atgtcaggatatttgaggtatccacatttgggattgtttaaagattaaatgaaatagtgtta | 156 |
| Plasmid | Sequence | aaagtatttaatatgcccttcaacaaatgatgaggaaatcttagaatctgctcagactcct | |
| | | tcagtttacatattaggaaactgaggcacagaaaggagcagagacttgctcaagtcca | |
| | | cccaaagcagtagagcattgtggttaaatgcaggacttcagtcagactgtctgggttca | |
| | | aatcctggttccacttggacatgggtttccttacataaatcacttcacctctctgagcctca | |
| | | gttttctcatatgcaaagtgaggataataataataccttccttacatggttactgatatgag | |
| | | tattaaatgtgccagctcatgtgcctggcgtataggaggtgctttataaaccttagctgtt | |
| | | accactcatggcattgccaaatgtgggacgggtctcctgactctctggtgtgagattga | |
| | | tggaatccacactttccagttcccttttctacctcctgggtatcttctcatatggttgtaagtt | |
| | | ccttggaggaagggaatgtggcttgctctctccaccacgctgagcatataagaggtgc | |
| | | tgaatgagcgcttttattcactcctctcatccccagccctcaccagctgggagttgttgta | |
| | | ggtgtcaattttctgcctctttccaacaccctgtgaggtgactgagcattgtcttccctccc | |
| | | aggcagctcacagtgtaagcttgtggacgatatcgaattcgcacgacattgattattga | |
| | | ctagtTATTAATAGTAATCAATTACGGGGTCATTAGTT | |
| | | CATAGCCCATATATGGAGTTCCGCGTTACATAACTT | |
| | | ACGGTAAATGGCCCGCCTGGCTGACCGCCCAACGA | |
| | | CCCCCGCCCATTGACGTCAATAATGACGTATGTTCC | |
| | | CATAGTAACGCCAATAGGGACTTTCCATTGACGTCA | |
| | | ATGGGTGGACTATTTACGGTAAACTGCCCACTTGGC | |
| | | AGTACATCAAGTGTATCATATGCCAAGTACGCCCCC | |
| | | TATTGACGTCAATGACGGTAAATGGCCCGCCTGGCA | |
| | | TTATGCCCAGTACATGACCTTATGGGACTTTCCTAC | |
| | | TTGGCAGTACATCTACGTATTAGTCATCGCTATTAC | |
| | | CATGGGTCGAGGTGAGCCCCACGTTCTGCTTCACTC | |
| | | TCCCCATCTCCCCCCCCTCCCCACCCCCAATTTTGTA | |
| | | TTTATTTATTTTTTAATTATTTTGTGCAGCGATGGGG | |
| | | GCGGGGGGGGGGGGGGCGCGCGCCAGGCGGGGCG | |
| | | GGGCGGGGCGAGGGGCGGGGCGGGGCGAGGCGGA | |
| | | GAGGTGCGGCGGCAGCCAATCAGAGCGGCGCGCTC | |
| | | CGAAAGTTTCCTTTTATGGCGAGGCGGCGGCGGCGG | |
| | | CGGCCCTATAAAAAGCGAAGCGCGCGGCGGGCGGG | |
| | | AGTCGCTGCGTTGCCTTCGCCCCGTGCCCCGCTCCG | |
| | | CGCCGCCTCGCGCCGCCCGCCCCGGCTCTGACTGAC | |
| | | CGCGTTACTCCCACAGGTGAGCGGGCGGGACGGCC | |
| | | CTTCTCCTCCGGGCTGTAATTAGCGCTTGGTTTAATG | |
| | | ACGGCTCGTTTCTTTTCTGTGGCTGCGTGAAAGCCTT | |
| | | AAAGGGCTCCGGGAGGGCCCTTTGTGCGGGGGGGA | |
| | | GCGGCTCGGGGGGTGCGTGCGTGTGTGTGTGCGTGG | |
| | | GGAGCGCCGCGTGCGGCCCGCGCTGCCCGGCGGCT | |
| | | GTGAGCGCTGCGGGCGCGGCGCGGGGCTTTGTGCG | |
| | | CTCCGCGTGTGCGCGAGGGGAGCGCGGCCGGGGGC | |
| | | GGTGCCCCGCGGTGCGGGGGGGCTGCGAGGGGAAC | |
| | | AAAGGCTGCGTGCGGGGTGTGTGCGTGGGGGGGTG | |
| | | AGCAGGGGGTGTGGGCGCGGCGGTCGGGCTGTAAC | |
| | | CCCCCCCTGCACCCCCCTCCCCGAGTTGCTGAGCAC | |
| | | GGCCCGGCTTCGGGTGCGGGGCTCCGTGCGGGGCG | |
| | | TGGCGCGGGGCTCGCCGTGCCGGGCGGGGGGTGGC | |
| | | GGCAGGTGGGGGTGCCGGGCGGGGCGGGGCCGCCT | |
| | | CGGGCCGGGGAGGGCTCGGGGGAGGGGCGCGGCG | |
| | | GCCCCGGAGCGCCGGCGGCTGTCGAGGCGCGGCGA | |
| | | GCCGCAGCCATTGCCTTTTATGGTAATCGTGCGAGA | |
| | | GGGCGCAGGGACTTCCTTTGTCCCAAATCTGGCGGA | |
| | | GCCGAAATCTGGGAGGCGCCGCCGCACCCCCTCTA | |
| | | GCGGGCGCGGGCGAAGCGGTGCGGCGCCGGCAGGA | |
| | | AGGAAATGGGCGGGGAGGGCCTTCGTGCGTCGCCG | |
| | | CGCCGCCGTCCCCTTCTCCATCTCCAGCCTCGGGGC | |
| | | TGCCGCAGGGGGACGGCTGCCTTCGGGGGGGACGG | |
| | | GGCAGGGCGGGGTTCGGCTTCTGGCGTGTGACCGG | |
| | | CGGCTCTAGAGCCTCTGCTAACCATGTTCATGCCTT | |
| | | CTTCTTTTTCCTACAGgggggatccgtttatctgcagaattcgcccttga | |
| | | cgtcgccaccATGGCGCTTCCGGTGACAGCACTGCTCCTC | |
| | | CCCTTGGCGCTGTTGCTCCACGCAGCAAGGCCGCAA | |
| | | GTGCAGCTCGTGCAAAGCGGCGGCGGACTGGTGCA | |
| | | ACCCGGCGGATCTCTGAGACTGAGCTGCGCCGCCA | |
| | | GCTACTTCGACTTCGACAGCTACGAGATGAGCTGGG | |
| | | TGAGGCAAGCCCCCGGAAAAGGACTGGAGTGGATC | |
| | | GGCAGCATCTACCACAGCGGCTCCACCTACTACAAC | |
| | | CCCTCTCTGAAATCTAGAGTGACCATCTCTAGAGAC | |
| | | AACTCCAAGAACACACTGTACCTCCAGATGAACAC | |
| | | ACTGAGAGCCGAGGACACCGCCACCTATTACTGCG | |
| | | CTAGAGTGAACATGGACAGATTCGACTACTGGGGC | |
| | | CAAGGCACACTGGTGACAGTGAGCTCCAGTGCTGCT | |
| | | GCCTTTGTCCCGGTATTTCTCCCAGCCAAACCGACC | |
| | | ACGACTCCCGCCCCGCGCCCTCCGACACCCGCTCCC | |
| | | ACCATCGCCTCTCAACCTCTTAGTCTTCGCCCCGAG | |
| | | GCATGCCGACCCGCCGCCGGGGGTGCTGTTCATACG | |
| | | AGGGGCTTGGACTTCGCTTGTGATATTTACATTTGG | |
| | | GCTCCGTTGGCGGGTACGTGCGGCGTCCTTTTGTTG | |
| | | TCACTCGTTATTACTTTGTATTGTAATCACAGGAATC | |
| | | GCTCAAAGCGGAGTAGGTTGTTGCATTCCGATTACA | |
| | | TGAATATGACTCCTCGCCGGCCTGGGCCGACAAGA | |
| | | AAACATTACCAACCCTATGCCCCCCCACGAGACTTC | |
| | | GCTGCGTACAGGTCCCGAGTGAAGTTTTCCCGAAGC | |
| | | GCAGACGCTCCGGCATATCAGCAAGGACAGAATCA | |
| | | GCTGTATAACGAACTGAATTTGGGACGCCGCGAGG | |
| | | AGTATGACGTGCTTGATAAACGCCGGGGGAGAGAC | |
| | | CCGGAAATGGGGGGTAAACCCCGAAGAAAGAATCC | |
| | | CCAAGAAGGACTCTACAATGAACTCCAGAAGGATA | |
| | | AGATGGCGGAGGCCTACTCAGAAATAGGTATGAAG | |
| | | GGCGAACGACGACGGGGAAAAGGTCACGATGGCCT | |
| | | CTACCAAGGGTTGAGTACGGCAACCAAAGATACGT | |
| | | ACGATGCACTGCATATGCAGGCCCTGCCTCCCAGAG | |
| | | GAAGCGGAGCTACTAACTTCAGCCTGCTGAAGCAG | |
| | | GCTGGAGACGTGGAGGAGAACCCTGGACCTatgtctcgc | |
| | | tccgttgccttagctgtgctcgcgctactctctctttctggattagaggctgtcatggcgc | |
| | | cccgaaccctcttcctgGGTGGAGGCGGTTCAGGCGGAGGTG | |
| | | GCTCTGGCGGTGGCGGATCGatccagcgtactccaaagattcaggt | |
| | | ttactcacgtcatccagcagagaatggaaagtcaaatttcctgaattgctatgtgtctgg | |
| | | gtttcatccatccgacattgaagttgacttactgaagaatggagagagaattgaaaaag | |
| | | tggagcattcagacttgtctttcagcaaggactggtctttctatctcttgtactacactgaa | |
| | | ttcacccccactgaaaaagatgagtatgcctgccgtgtgaaccatgtgactttgtcaca | |
| | | gcccaagatagttaagtgggatcgagacatgGGTGGTGGTGGTTCTG | |
| | | GTGGTGGTGGTTCTGGCGGCGGCGGCTCCGGTGGTG | |
| | | GTGGATCCggctcccactccttgaagtatttccacacttccgtgtcccggcccg | |
| | | gccgcggggagccccgcttcatctctgtgggctacgtggacgacacccagttcgtgc | |
| | | gcttcgacaacgacgccgcgagtccgaggatggtgccgcgggcgccgtggatgga | |
| | | gcaggaggggtcagagtattgggaccgggagacacggagcgccagggacaccgc | |
| | | acagattttccgagtgaatctgcggacgctgcgcggctactacaatcagagcgaggc | |
| | | cgggtctcacaccctgcagtggatgcatggctgcgagctggggcccgacgggcgct | |
| | | tcctccgcgggtatgaacagttcgcctacgacggcaaggattatctcaccctgaatga | |
| | | ggacctgcgctcctggaccgcggtggacacggcggctcagatctccgagcaaaagt | |
| | | caaatgatgcctctgaggcggagcaccagagagcctacctggaagacacatgcgtg | |
| | | gagtggctccacaaatacctggagaaggggaaggagacgctgcttcacctggagcc | |
| | | cccaaagacacacgtgactcaccaccccatctctgaccatgaggccaccctgaggtg | |
| | | ctgggccctgggcttctaccctgcggagatcacactgacctggcagcaggatgggg | |
| | | agggccatacccaggacacggagctcgtggagaccaggcctgcaggggatggaa | |
| | | ccttccagaagtgggcagctgtggtggtgccttctggagaggagcagagatacacgt | |
| | | gccatgtgcagcatgaggggctacccgagcccgtcaccctgagatggaagccggct | |
| | | tcccagcccaccatccccatcgtgggcatcattgctggcctggttctccttggatctgtg | |
| | | gtctctggagctgtggttgctgctgtgatatggaggaagaagagctcaggtggaaaag | |
| | | gagggagctactctaaggctgagtggagcgacagtgcccaggggtctgagtctcac | |
| | | agcttgtaatgatagCCGCTGATCAGCCTCGACTGTGCCTTC | |
| | | TAGTTGCCAGCCATCTGTTGTTTGCCCCTCCCCCGTG | |
| | | CCTTCCTTGACCCTGGAAGGTGCCACTCCCACTGTC | |
| | | CTTTCCTAATAAAATGAGGAAATTGCATCGCATTGT | |
| | | CTGAGTAGGTGTCATTCTATTCTGGGGGGTGGGGTG | |
| | | GGGCAGGACAGCAAGGGGGAGGATTGGGAAGACA | |
| | | ATAGCAGGCATGCTGGGGATGCGGTGGGCTCTATG | |
| | | GGTCGACtgaccagatggacctggctggagaagaagagattgagctctactca | |
| | | ggtgggccctcctccctctggtctcttccggtatcccccacccctcagcttgctgtaga | |
| | | gacggcaatcaggggaaattctggtccctgccctcccgtcagcaccacggacagctc | |
| | | ccacgtctgtgggacgctctctgcagatggggatgatctcccagccctgccccgcctc | |
| | | tccctcgttccccaccagccctctttccagaaatttccttcttcatccaagggacttttcct | |
| | | cccagaacccgacacagacaccatcaactgcgaccagttcagcaggctgttgtgtga | |
| | | catggaaggtgatgaagagaccagggaggcttatgccaatatcggtgaggaagcac | |
| | | ctgagcccagaaaaggacaatcaagggcaagagttctttgctgccacttgtcaatatc | |
| | | acccattcatcatgagccacgtcagtcccctcccacagaaatcattgcaagggggatg | |
| | | cggagcaatggctggaggaacggagactccagggaagagaggggagatggagg | |
| | | ccagtgggggaaataggccccttcactaatgaccaccaagaaaacaaaatctcatgtt | |
| | | tacatcctccacctccatttctatacgcatttctgcttcttgctcttctgtccatcctttctaca | |
| | | aagcccataccatacacccctttcccttttcctcccagctccttagccaagctactctagt | |
| | | atttgtaataactagcatttactggatactcatagtatgctcattgctgtccGGTAAC | |
| | | CACGTGCGGACCGggctccggtgcccgtcagtgggcagagcgcacatc | |
| | | gcccacagtccccgagaagttggggggaggggtcggcaattgaaccggtgcctag | |
| | | agaaggtggcgcggggtaaactgggaaagtgatgtcgtgtactggctccgcctttttc | |
| | | ccgagggtgggggagaaccgtatataagtgcagtagtcgccgtgaacgttctttttcg | |
| | | caacgggtttgccgccagaacacaggtaagtgccgtgtgtggttcccgcgggcctgg | |
| | | cctctttacgggttatggcccttgcgtgccttgaattacttccactggctgcagtacgtga | |
| | | ttcttgatcccgagcttcgggttggaagtgggtgggagagttcgaggccttgcgcttaa | |
| | | ggagccccttcgcctcgtgcttgagttgaggcctggcctgggcgctggggccgccg | |
| | | cgtgcgaatctggtggcaccttcgcgcctgtctcgctgctttcgataagtctctagccat | |
| | | ttaaaatttttgatgacctgctgcgacgctttttttctggcaagatagtcttgtaaatgcgg | |
| | | gccaagatctgcacactggtatttcggtttttggggccgcgggcggcgacggggccc | |
| | | gtgcgtcccagcgcacatgttcggcgaggcggggcctgcgagcgcggccaccgag | |
| | | aatcggacgggggtagtctcaagctggccggcctgctctggtgcctggcctcgcgcc | |
| | | gccgtgtatcgccccgccctgggcggcaaggctggcccggtcggcaccagttgcgt | |
| | | gagcggaaagatggccgcttcccggccctgctgcagggagctcaaaatggaggac | |
| | | gcggcgctcgggagagcgggcgggtgagtcacccacacaaaggaaaagggccttt | |
| | | ccgtcctcagccgtcgcttcatgtgactccacggagtaccgggcgccgtccaggcac | |
| | | ctcgattagttctcgagcttttggagtacgtcgtctttaggttggggggaggggttttatg | |
| | | cgatggagtttccccacactgagtgggtggagactgaagttaggccagcttggcactt | |
| | | gatgtaattctccttggaatttgccctttttgagtttggatcttggttcattctcaagcctcag | |
| | | acagtggttcaaagtttttttcttccatttcaggtgtcgtgacttgacgtcgccaccatgag | |
| | | gatatttgctgtctttatattcatgacctactggcatttgctgaacgcatttactgtcacggt | |
| | | tcccaaggacctatatgtggtagagtatggtagcaatatgacaattgaatgcaaattccc | |
| | | agtagaaaaacaattagacctggctgcactaattgtctattgggaaatggaggataaga | |
| | | acattattcaatttgtgcatggagaggaagacctgaaggttcagcatagtagctacaga | |
| | | cagagggcccggctgttgaaggaccagctctccctgggaaatgctgcacttcagatc | |
| | | acagatgtgaaattgcaggatgcaggggtgtaccgctgcatgatcagctatggtggtg | |
| | | ccgactacaagcgaattactgtgaaagtcaatgccccatacaacaaaatcaaccaaag | |
| | | aattttggttgtggatccagtcacctctgaacatgaactgacatgtcaggctgagggcta | |
| | | ccccaaggccgaagtcatctggacaagcagtgaccatcaagtcctgagtggtaagac | |
| | | caccaccaccaattccaagagagaggagaaacttttcaatgtgaccagcacactgag | |
| | | aatcaacacaacaactaatgagattttctactgcacttttaggagattagatcctgagga | |
| | | aaaccatacagctgaattggtcatcccagaactacctctggcacatcctccaaatgaaa | |
| | | ggactcacttggtaattctgggagccatcttattatgccttggtgtagcactgacattcat | |
| | | cttccgtttaagaaaagggagaatgatggatgtgaaaaaatgtggcatccaagataca | |
| | | aactcaaagaagcaaagtgatacacatttggaggagacgtaaCCGCTGATC | |
| | | AGCCTCGAAACTTGTTTATTGCAGCTTATAATGGTT | |
| | | ACAAATAAAGCAATAGCATCACAAATTTCACAAAT | |
| | | AAAGCATTTTTTTCACTGCATTCTAGTTGTGGTTTGT | |
| | | CCAAACTCATCAATGTATCTTAGGCGCCTGATGCGG | |
| | | TATTTTCTCCTTACGCATCTGTGCGGTATTTCACACC | |
| | | GCATACAGTACTGTCAAAGCAACCATAGTACGCGC | |
| | | CCTGTAGCGGCGCATTAAGCGCGGCGGGTGTGGTG | |
| | | GTTACGCGCAGCGTGACCGCTACACTTGCCAGCGCC | |
| | | CTAGCGCCCGCTCCTTTCGCTTTCTTCCCTTCCTTTC | |
| | | TCGCCACGTTCGCCGGCTTTCCCCGTCAAGCTCTAA | |
| | | ATCGGGGGCTCCCTTTAGGGTTCCGATTTAGTGCTT | |
| | | TACGGCACCTCGACCCCAAAAAACTTGATTTGGGTG | |
| | | ATGGTTCACGTAGTGGGCCATCGCCCTGATAGACGG | |
| | | TTTTTCGCCCTTTGACGTTGGAGTCCACGTTCTTTAA | |
| | | TAGTGGACTCTTGTTCCAAACTGGAACAACACTCAA | |
| | | CCCTATCTCGGGCTATTCTTTTGATTTATAAGGGATT | |
| | | TTGCCGATTTCGGCCTATTGGTTAAAAAATGAGCTG | |
| | | ATTTAACAAAAATTTAACGCGAATTTTAACAAAATA | |
| | | TTAACGTTTACAATTTTATGGTGCACTCTCAGTACA | |
| | | ATCTGCTCTGATGCCGCATAGTTAAGCCAGCCCCGA | |
| | | CACCCGCCAACACCCGCTGACGCGCCCTGACGGGCT | |
| | | TGTCTGCTCCCGGCATCCGCTTACAGACAAGCTGTG | |
| | | ACCGTCTCCGGGAGCTGCATGTGTCAGAGGTTTTCA | |
| | | CCGTCATCACCGAAACGCGCGAGACGAAAGGGCCT | |
| | | CGTGATACGCCTATTTTTATAGGTTAATGTCATGAA | |
| | | CAATAAAACTGTCTGCTTACATAAACAGTAATACAA | |
| | | GGGGTGTTATGAGCCATATTCAACGGGAAACGTCG | |
| | | AGGCCGCGATTAAATTCCAACATGGATGCTGATTTA | |
| | | TATGGGTATAAATGGGCTCGCGATAATGTCGGGCA | |
| | | ATCAGGTGCGACAATCTATCGCTTGTATGGGAAGCC | |
| | | CGATGCGCCAGAGTTGTTTCTGAAACATGGCAAAG | |
| | | GTAGCGTTGCCAATGATGTTACAGATGAGATGGTCA | |
| | | GACTAAACTGGCTGACGGAATTTATGCCTCTTCCGA | |
| | | CCATCAAGCATTTTATCCGTACTCCTGATGATGCAT | |
| | | GGTTACTCACCACTGCGATCCCCGGAAAAACAGCAT | |
| | | TCCAGGTATTAGAAGAATATCCTGATTCAGGTGAAA | |
| | | ATATTGTTGATGCGCTGGCAGTGTTCCTGCGCCGGT | |
| | | TGCATTCGATTCCTGTTTGTAATTGTCCTTTTAACAG | |
| | | CGATCGCGTATTTCGTCTCGCTCAGGCGCAATCACG | |
| | | AATGAATAACGGTTTGGTTGATGCGAGTGATTTTGA | |
| | | TGACGAGCGTAATGGCTGGCCTGTTGAACAAGTCTG | |
| | | GAAAGAAATGCATAAACTTTTGCCATTCTCACCGGA | |
| | | TTCAGTCGTCACTCATGGTGATTTCTCACTTGATAAC | |
| | | CTTATTTTTGACGAGGGGAAATTAATAGGTTGTATT | |
| | | GATGTTGGACGAGTCGGAATCGCAGACCGATACCA | |
| | | GGATCTTGCCATCCTATGGAACTGCCTCGGTGAGTT | |
| | | TTCTCCTTCATTACAGAAACGGCTTTTTCAAAAATA | |
| | | TGGTATTGATAATCCTGATATGAATAAATTGCAGTT | |
| | | TCATTTGATGCTCGATGAGTTTTTCTAATCTCATGAC | |
| | | CAAAATCCCTTAACGTGAGTTTTCGTTCCACTGAGC | |
| | | GTCAGACCCCGTAGAAAAGATCAAAGGATCTTCTTG | |
| | | AGATCCTTTTTTTCTGCGCGTAATCTGCTGCTTGCAA | |
| | | ACAAAAAAACCACCGCTACCAGCGGTGGTTTGTTTG | |
| | | CCGGATCAAGAGCTACCAACTCTTTTTCCGAAGGTA | |
| | | ACTGGCTTCAGCAGAGCGCAGATACCAAATACTGTC | |
| | | CTTCTAGTGTAGCCGTAGTTAGGCCACCACTTCAAG | |
| | | AACTCTGTAGCACCGCCTACATACCTCGCTCTGCTA | |
| | | ATCCTGTTACCAGTGGCTGCTGCCAGTGGCGATAAG | |
| | | TCGTGTCTTACCGGGTTGGACTCAAGACGATAGTTA | |
| | | CCGGATAAGGCGCAGCGGTCGGGCTGAACGGGGGG | |
| | | TTCGTGCACACAGCCCAGCTTGGAGCGAACGACCTA | |
| | | CACCGAACTGAGATACCTACAGCGTGAGCTATGAG | |
| | | AAAGCGCCACGCTTCCCGAAGGGAGAAAGGCGGAC | |
| | | AGGTATCCGGTAAGCGGCAGGGTCGGAACAGGAGA | |
| | | GCGCACGAGGGAGCTTCCAGGGGGAAACGCCTGGT | |
| | | ATCTTTATAGTCCTGTCGGGTTTCGCCACCTCTGACT | |
| | | TGAGCGTCGATTTTTGTGATGCTCGTCAGGGGGGCG | |
| | | GAGCCTATGGAAAAACGCCAGCAACGCGGCCTTTTT | |
| | | ACGGTTCCTGGCCTTTTGCTGGCCTTTTGCTCACATG | |
| | | TGCGGCCGCACGCGTcatatttatggggtatatgtgaatatttattacatgc | |
| | | atagaaggtataatgatc | |
| GPC3 | Amino Acid | MALPVTALLLPLALLLHAARPQVQLVQSGGGLVQPG | 157 |
| CAR | Sequence | GSLRLSCAASYFDFDSYEMSWVRQAPGKGLEWIGSIY | |
| | | HSGSTYYNPSLKSRVTISRDNSKNTLYLQMNTLRAED | |
| | | TATYYCARVNMDRFDYWGQGTLVTVSSSAAAFVPVF | |
| | | LPAKPTTTPAPRPPTPAPTIASQPLSLRPEACRPAAGGA | |
| | | VHTRGLDFACDIYIWAPLAGTCGVLLLSLVITLYCNHR | |
| | | NRSKRSRLLHSDYMNMTPRRPGPTRKHYQPYAPPRDF | |
| | | AAYRSRVKFSRSADAPAYQQGQNQLYNELNLGRREE | |
| | | YDVLDKRRGRDPEMGGKPRRKNPQEGLYNELQKDK | |
| | | MAEAYSEIGMKGERRRGKGHDGLYQGLSTATKDTYD | |
| | | ALHMQALPPR | |
| GPR87 | Nucleic Acid | tgcatagaaggtataatgatcatgtcaggatatttgaggtatccacatttgggattgtttaa | 158 |
| CAR donor | Sequence | agattaaatgaaatagtgttaaaagtatttaatatgcccttcaacaaatgatgaggaaatc | |
| plasmid | | ttagaatctgctcagactccttcagtttacatattaggaaactgaggcacagaaaggag | |
| | | cagagacttgctcaagtccacccaaagcagtagagcattgtggttaaatgcaggactt | |
| | | cagtcagactgtctgggttcaaatcctggttccacttggacatgggtttccttacataaat | |
| | | cacttcacctctctgagcctcagttttctcatatgcaaagtgaggataataataataccttc | |
| | | cttacatggttactgatatgagtattaaatgtgccagctcatgtgcctggcgtataggag | |
| | | gtgctttataaaccttagctgttaccactcatggcattgccaaatgtgggacgggtctcct | |
| | | gactctctggtgtgagattgatggaatccacactttccagttcccttttctacctcctgggt | |
| | | atcttctcatatggttgtaagttccttggaggaagggaatgtggcttgctctctccaccac | |
| | | gctgagcatataagaggtgctgaatgagcgcttttattcactcctctcatccccagccct | |
| | | caccagctgggagttgttgtaggtgtcaattttctgcctctttccaacaccctgtgaggtg | |
| | | actgagcattgtcttccctcccaggcagctcacagtgtaagcttgtggacgatatcgaa | |
| | | ttcgcacgacattgattattgactagtTATTAATAGTAATCAATTAC | |
| | | GGGGTCATTAGTTCATAGCCCATATATGGAGTTCCG | |
| | | CGTTACATAACTTACGGTAAATGGCCCGCCTGGCTG | |
| | | ACCGCCCAACGACCCCCGCCCATTGACGTCAATAAT | |
| | | GACGTATGTTCCCATAGTAACGCCAATAGGGACTTT | |
| | | CCATTGACGTCAATGGGTGGACTATTTACGGTAAAC | |
| | | TGCCCACTTGGCAGTACATCAAGTGTATCATATGCC | |
| | | AAGTACGCCCCCTATTGACGTCAATGACGGTAAATG | |
| | | GCCCGCCTGGCATTATGCCCAGTACATGACCTTATG | |
| | | GGACTTTCCTACTTGGCAGTACATCTACGTATTAGT | |
| | | CATCGCTATTACCATGGGTCGAGGTGAGCCCCACGT | |
| | | TCTGCTTCACTCTCCCCATCTCCCCCCCCTCCCCACC | |
| | | CCCAATTTTGTATTTATTTATTTTTTAATTATTTTGTG | |
| | | CAGCGATGGGGGCGGGGGGGGGGGGGGCGCGCGC | |
| | | CAGGCGGGGCGGGGCGGGGCGAGGGGCGGGGCGG | |
| | | GGCGAGGCGGAGAGGTGCGGCGGCAGCCAATCAGA | |
| | | GCGGCGCGCTCCGAAAGTTTCCTTTTATGGCGAGGC | |
| | | GGCGGCGGCGGCGGCCCTATAAAAAGCGAAGCGCG | |
| | | CGGCGGGGGGAGTCGCTGCGTTGCCTTCGCCCCGT | |
| | | GCCCCGCTCCGCGCCGCCTCGCGCCGCCCGCCCCGG | |
| | | CTCTGACTGACCGCGTTACTCCCACAGGTGAGCGGG | |
| | | CGGGACGGCCCTTCTCCTCCGGGCTGTAATTAGCGC | |
| | | TTGGTTTAATGACGGCTCGTTTCTTTTCTGTGGCTGC | |
| | | GTGAAAGCCTTAAAGGGCTCCGGGAGGGCCCTTTGT | |
| | | GCGGGGGGGAGCGGCTCGGGGGGTGCGTGCGTGTG | |
| | | TGTGTGCGTGGGGAGCGCCGCGTGCGGCCCGCGCT | |
| | | GCCCGGCGGCTGTGAGCGCTGCGGGCGCGGCGCGG | |
| | | GGCTTTGTGCGCTCCGCGTGTGCGCGAGGGGAGCGC | |
| | | GGCCGGGGGCGGTGCCCCGCGGTGCGGGGGGGCTG | |
| | | CGAGGGGAACAAAGGCTGCGTGCGGGGTGTGTGCG | |
| | | TGGGGGGGTGAGCAGGGGGTGTGGGCGCGGCGGTC | |
| | | GGGCTGTAACCCCCCCCTGCACCCCCCTCCCCGAGT | |
| | | TGCTGAGCACGGCCCGGCTTCGGGTGCGGGGCTCCG | |
| | | TGCGGGGCGTGGCGCGGGGCTCGCCGTGCCGGGCG | |
| | | GGGGGTGGCGGCAGGTGGGGGTGCCGGGCGGGGCG | |
| | | GGGCCGCCTCGGGCCGGGGAGGGCTCGGGGGAGGG | |
| | | GCGCGGCGGCCCCGGAGCGCCGGCGGCTGTCGAGG | |
| | | CGCGGCGAGCCGCAGCCATTGCCTTTTATGGTAATC | |
| | | GTGCGAGAGGGCGCAGGGACTTCCTTTGTCCCAAAT | |
| | | CTGGCGGAGCCGAAATCTGGGAGGCGCCGCCGCAC | |
| | | CCCCTCTAGCGGGCGCGGGCGAAGCGGTGCGGCGC | |
| | | CGGCAGGAAGGAAATGGGCGGGGAGGGCCTTCGTG | |
| | | CGTCGCCGCGCCGCCGTCCCCTTCTCCATCTCCAGC | |
| | | CTCGGGGCTGCCGCAGGGGGACGGCTGCCTTCGGG | |
| | | GGGGACGGGGCAGGGCGGGGTTCGGCTTCTGGCGT | |
| | | GTGACCGGCGGCTCTAGAGCCTCTGCTAACCATGTT | |
| | | CATGCCTTCTTCTTTTTCCTACAGgggggatccgtttatctgcag | |
| | | aattcgcccttgacgtcgccaccATGGCCTTACCAGTGACCGCCT | |
| | | TGCTCCTGCCGCTGGCCTTGCTGCTCCACGCCGCCA | |
| | | GGCCGGACGTGGTCATGACCCAGACCCCTCTGAGCC | |
| | | TGCCCGTGAGCCTGGGCGACCAGGCTTCCATCTCCT | |
| | | GCAGAAGCTCCCAGAGCCTGGTGCACAGCAGCGGC | |
| | | AACACCTACCTGCACTGGTACCTGCAGAAGCCCGGC | |
| | | CAGAGCCCCAAGCTGCTGATCTACAAGGTGTCCAAC | |
| | | AGATTTTCCGGCGTGCCCGACAGATTTTCCGGATCC | |
| | | GGCAGCGGCACCGACTTCACACTGAAGATCAGCAG | |
| | | AGTGGAGGCCGAGGACCTGGGCGTGTACTTCTGTA | |
| | | GCCAGAGCACACACGTGCCTTACACCTTTGGCGGCG | |
| | | GCACAAAGCTGGAGATCAAGGGCGGCGGCGGCTCC | |
| | | GGAGGAGGAGGAAGCGGAGGAGGCGGCTCCGAGG | |
| | | TGCAGCTGCAGCAGTCCGGCCCTGACCTGGTGAAGC | |
| | | CTGGCGCCTCCATGAAGATCAGCTGCAAGGCCAGC | |
| | | GGCTACTCCTTCACCGATTACACCATGCACTGGGTG | |
| | | AAGCAGAGCCACGGCAAGAATTTCGAGTGGATCGG | |
| | | CCTGATCAATCCCTACAATGACGGCACCACCTACAA | |
| | | TCAGAAGTTCAAGGGCAAGGCCACACTGACAGTGG | |
| | | ATAAGAGCAGCTCCACAGCCTACATGGAGCTGCTG | |
| | | AGCCTGACAAGCGAGGATTCCGCCGTGTACTACTGC | |
| | | GCCTCCCTGGACTACTGGGGCCAGGGCACCTCCGTG | |
| | | ACCGTGTCCTCCACCACTACCCCAGCACCGAGGCCA | |
| | | CCCACCCCGGCTCCTACCATCGCCTCCCAGCCTCTG | |
| | | TCCCTGCGTCCGGAGGCATGTAGACCCGCAGCTGGT | |
| | | GGGGCCGTGCATACCCGGGGTCTTGACTTCGCCTGC | |
| | | GATTTCTGGGTGCTGGTCGTTGTGGGCGGCGTGCTG | |
| | | GCCTGCTACAGCCTGCTGGTGACAGTGGCCTTCATC | |
| | | ATCTTTTGGGTGAGGAGCAAGCGGAGCAGACTGCT | |
| | | GCACAGCGACTACATGAACATGACCCCCCGGAGGC | |
| | | CTGGCCCCACCCGGAAGCACTACCAGCCCTACGCCC | |
| | | CTCCCAGGGATTTCGCCGCCTACCGGAGCCGCGTGA | |
| | | AATTCAGCCGCAGCGCAGATGCTCCAGCCTACAAG | |
| | | CAGGGGCAGAACCAGCTCTACAACGAACTCAATCT | |
| | | TGGTCGGAGAGAGGAGTACGACGTGCTGGACAAGC | |
| | | GGAGAGGACGGGACCCAGAAATGGGCGGGAAGCC | |
| | | GCGCAGAAAGAATCCCCAAGAGGGCCTGTACAACG | |
| | | AGCTCCAAAAGGATAAGATGGCAGAAGCCTATAGC | |
| | | GAGATTGGTATGAAAGGGGAACGCAGAAGAGGCAA | |
| | | AGGCCACGACGGACTGTACCAGGGACTCAGCACCG | |
| | | CCACCAAGGACACCTATGACGCTCTTCACATGCAGG | |
| | | CCCTGCCGCCTCGGGGAAGCGGAGCTACTAACTTCA | |
| | | GCCTGCTGAAGCAGGCTGGAGACGTGGAGGAGAAC | |
| | | CCTGGACCTatgtctcgctccgttgccttagctgtgctcgcgctactctctctttct | |
| | | ggattagaggctgtcatggcgccccgaaccctcttcctgGGTGGAGGCGG | |
| | | TTCAGGCGGAGGTGGCTCTGGCGGTGGCGGATCGatc | |
| | | cagcgtactccaaagattcaggtttactcacgtcatccagcagagaatggaaagtcaa | |
| | | atttcctgaattgctatgtgtctgggtttcatccatccgacattgaagttgacttactgaag | |
| | | aatggagagagaattgaaaaagtggagcattcagacttgtctttcagcaaggactggt | |
| | | ctttctatctcttgtactacactgaattcacccccactgaaaaagatgagtatgcctgccg | |
| | | tgtgaaccatgtgactttgtcacagcccaagatagttaagtgggatcgagacatgGG | |
| | | TGGTGGTGGTTCTGGTGGTGGTGGTTCTGGCGGCGG | |
| | | CGGCTCCGGTGGTGGTGGATCCggctcccactccttgaagtatttc | |
| | | cacacttccgtgtcccggcccggccgcggggagccccgcttcatctctgtgggctac | |
| | | gtggacgacacccagttcgtgcgcttcgacaacgacgccgcgagtccgaggatggt | |
| | | gccgcgggcgccgtggatggagcaggaggggtcagagtattgggaccgggagac | |
| | | acggagcgccagggacaccgcacagattttccgagtgaatctgcggacgctgcgcg | |
| | | gctactacaatcagagcgaggccgggtctcacaccctgcagtggatgcatggctgcg | |
| | | agctggggcccgacgggcgcttcctccgcgggtatgaacagttcgcctacgacggc | |
| | | aaggattatctcaccctgaatgaggacctgcgctcctggaccgcggtggacacggcg | |
| | | gctcagatctccgagcaaaagtcaaatgatgcctctgaggcggagcaccagagagc | |
| | | ctacctggaagacacatgcgtggagtggctccacaaatacctggagaaggggaagg | |
| | | agacgctgcttcacctggagcccccaaagacacacgtgactcaccaccccatctctg | |
| | | accatgaggccaccctgaggtgctgggccctgggcttctaccctgcggagatcacac | |
| | | tgacctggcagcaggatggggagggccatacccaggacacggagctcgtggagac | |
| | | caggcctgcaggggatggaaccttccagaagtgggcagctgtggtggtgccttctgg | |
| | | agaggagcagagatacacgtgccatgtgcagcatgaggggctacccgagcccgtc | |
| | | accctgagatggaagccggcttcccagcccaccatccccatcgtgggcatcattgctg | |
| | | gcctggttctccttggatctgtggtctctggagctgtggttgctgctgtgatatggagga | |
| | | agaagagctcaggtggaaaaggagggagctactctaaggctgagtggagcgacag | |
| | | tgcccaggggtctgagtctcacagcttgtaatgatagCCGCTGATCAGCC | |
| | | TCGACTGTGCCTTCTAGTTGCCAGCCATCTGTTGTTT | |
| | | GCCCCTCCCCCGTGCCTTCCTTGACCCTGGAAGGTG | |
| | | CCACTCCCACTGTCCTTTCCTAATAAAATGAGGAAA | |
| | | TTGCATCGCATTGTCTGAGTAGGTGTCATTCTATTCT | |
| | | GGGGGGTGGGGTGGGGCAGGACAGCAAGGGGGAG | |
| | | GATTGGGAAGACAATAGCAGGCATGCTGGGGATGC | |
| | | GGTGGGCTCTATGGGTCGACtgaccagatggacctggctggagaa | |
| | | gaagagattgagctctactcaggtgggccctcctccctctggtctcttccggtatcccc | |
| | | cacccctcagcttgctgtagagacggcaatcaggggaaattctggtccctgccctccc | |
| | | gtcagcaccacggacagctcccacgtctgtgggacgctctctgcagatggggatgat | |
| | | ctcccagccctgccccgcctctccctcgttccccaccagccctctttccagaaatttcct | |
| | | tcttcatccaagggacttttcctcccagaacccgacacagacaccatcaactgcgacc | |
| | | agttcagcaggctgttgtgtgacatggaaggtgatgaagagaccagggaggcttatg | |
| | | ccaatatcggtgaggaagcacctgagcccagaaaaggacaatcaagggcaagagtt | |
| | | ctttgctgccacttgtcaatatcacccattcatcatgagccacgtcagtcccctcccaca | |
| | | gaaatcattgcaagggggatgcggagcaatggctggaggaacggagactccaggg | |
| | | aagagaggggagatggaggccagtgggggaaataggccccttcactaatgaccac | |
| | | caagaaaacaaaatctcatgtttacatcctccacctccatttctatacgcatttctgcttctt | |
| | | gctcttctgtccatcctttctacaaagcccataccatacacccctttcccttttcctcccag | |
| | | ctccttagccaagctactctagtatttgtaataactagcatttactggatactcatagtatg | |
| | | ctcattgctgtccGGTAACCACGTGCGGACCGggctccggtgcccg | |
| | | tcagtgggcagagcgcacatcgcccacagtccccgagaagttggggggaggggtc | |
| | | ggcaattgaaccggtgcctagagaaggtggcgcggggtaaactgggaaagtgatgt | |
| | | cgtgtactggctccgcctttttcccgaggggggggagaaccgtatataagtgcagta | |
| | | gtcgccgtgaacgttctttttcgcaacgggtttgccgccagaacacaggtaagtgccgt | |
| | | gtgtggttcccgcgggcctggcctctttacgggttatggcccttgcgtgccttgaattac | |
| | | ttccactggctgcagtacgtgattcttgatcccgagcttcgggttggaagtgggtggga | |
| | | gagttcgaggccttgcgcttaaggagccccttcgcctcgtgcttgagttgaggcctgg | |
| | | cctgggcgctggggccgccgcgtgcgaatctggtggcaccttcgcgcctgtctcgct | |
| | | gctttcgataagtctctagccatttaaaatttttgatgacctgctgcgacgctttttttctgg | |
| | | caagatagtcttgtaaatgcgggccaagatctgcacactggtatttcggtttttggggcc | |
| | | gcgggcggcgacggggcccgtgcgtcccagcgcacatgttcggcgaggcggggc | |
| | | ctgcgagcgcggccaccgagaatcggacgggggtagtctcaagctggccggcctg | |
| | | ctctggtgcctggcctcgcgccgccgtgtatcgccccgccctgggcggcaaggctg | |
| | | gcccggtcggcaccagttgcgtgagcggaaagatggccgcttcccggccctgctgc | |
| | | agggagctcaaaatggaggacgcggcgctcgggagagcgggcgggtgagtcacc | |
| | | cacacaaaggaaaagggcctttccgtcctcagccgtcgcttcatgtgactccacggag | |
| | | taccgggcgccgtccaggcacctcgattagttctcgagcttttggagtacgtcgtcttta | |
| | | ggttggggggaggggttttatgcgatggagtttccccacactgagtgggtggagactg | |
| | | aagttaggccagcttggcacttgatgtaattctccttggaatttgccctttttgagtttggat | |
| | | cttggttcattctcaagcctcagacagtggttcaaagtttttttcttccatttcaggtgtcgt | |
| | | gacttgacgtcgccaccatgaggatatttgctgtctttatattcatgacctactggcatttg | |
| | | ctgaacgcatttactgtcacggttcccaaggacctatatgtggtagagtatggtagcaat | |
| | | atgacaattgaatgcaaattcccagtagaaaaacaattagacctggctgcactaattgtc | |
| | | tattgggaaatggaggataagaacattattcaatttgtgcatggagaggaagacctgaa | |
| | | ggttcagcatagtagctacagacagagggcccggctgttgaaggaccagctctccct | |
| | | gggaaatgctgcacttcagatcacagatgtgaaattgcaggatgcaggggtgtaccg | |
| | | ctgcatgatcagctatggtggtgccgactacaagcgaattactgtgaaagtcaatgccc | |
| | | catacaacaaaatcaaccaaagaattttggttgtggatccagtcacctctgaacatgaa | |
| | | ctgacatgtcaggctgagggctaccccaaggccgaagtcatctggacaagcagtga | |
| | | ccatcaagtcctgagtggtaagaccaccaccaccaattccaagagagaggagaaact | |
| | | tttcaatgtgaccagcacactgagaatcaacacaacaactaatgagattttctactgcac | |
| | | ttttaggagattagatcctgaggaaaaccatacagctgaattggtcatcccagaactac | |
| | | ctctggcacatcctccaaatgaaaggactcacttggtaattctgggagccatcttattat | |
| | | gccttggtgtagcactgacattcatcttccgtttaagaaaagggagaatgatggatgtg | |
| | | aaaaaatgtggcatccaagatacaaactcaaagaagcaaagtgatacacatttggag | |
| | | gagacgtaaCCGCTGATCAGCCTCGAAACTTGTTTATTG | |
| | | CAGCTTATAATGGTTACAAATAAAGCAATAGCATCA | |
| | | CAAATTTCACAAATAAAGCATTTTTTTCACTGCATT | |
| | | CTAGTTGTGGTTTGTCCAAACTCATCAATGTATCTTA | |
| | | GGCGCCTGATGCGGTATTTTCTCCTTACGCATCTGT | |
| | | GCGGTATTTCACACCGCATACAGTACTGTCAAAGCA | |
| | | ACCATAGTACGCGCCCTGTAGCGGCGCATTAAGCGC | |
| | | GGCGGGTGTGGTGGTTACGCGCAGCGTGACCGCTA | |
| | | CACTTGCCAGCGCCCTAGCGCCCGCTCCTTTCGCTTT | |
| | | CTTCCCTTCCTTTCTCGCCACGTTCGCCGGCTTTCCC | |
| | | CGTCAAGCTCTAAATCGGGGGCTCCCTTTAGGGTTC | |
| | | CGATTTAGTGCTTTACGGCACCTCGACCCCAAAAAA | |
| | | CTTGATTTGGGTGATGGTTCACGTAGTGGGCCATCG | |
| | | CCCTGATAGACGGTTTTTCGCCCTTTGACGTTGGAG | |
| | | TCCACGTTCTTTAATAGTGGACTCTTGTTCCAAACTG | |
| | | GAACAACACTCAACCCTATCTCGGGCTATTCTTTTG | |
| | | ATTTATAAGGGATTTTGCCGATTTCGGCCTATTGGTT | |
| | | AAAAAATGAGCTGATTTAACAAAAATTTAACGCGA | |
| | | ATTTTAACAAAATATTAACGTTTACAATTTTATGGT | |
| | | GCACTCTCAGTACAATCTGCTCTGATGCCGCATAGT | |
| | | TAAGCCAGCCCCGACACCCGCCAACACCCGCTGAC | |
| | | GCGCCCTGACGGGCTTGTCTGCTCCCGGCATCCGCT | |
| | | TACAGACAAGCTGTGACCGTCTCCGGGAGCTGCATG | |
| | | TGTCAGAGGTTTTCACCGTCATCACCGAAACGCGCG | |
| | | AGACGAAAGGGCCTCGTGATACGCCTATTTTTATAG | |
| | | GTTAATGTCATGAACAATAAAACTGTCTGCTTACAT | |
| | | AAACAGTAATACAAGGGGTGTTATGAGCCATATTC | |
| | | AACGGGAAACGTCGAGGCCGCGATTAAATTCCAAC | |
| | | ATGGATGCTGATTTATATGGGTATAAATGGGCTCGC | |
| | | GATAATGTCGGGCAATCAGGTGCGACAATCTATCGC | |
| | | TTGTATGGGAAGCCCGATGCGCCAGAGTTGTTTCTG | |
| | | AAACATGGCAAAGGTAGCGTTGCCAATGATGTTAC | |
| | | AGATGAGATGGTCAGACTAAACTGGCTGACGGAAT | |
| | | TTATGCCTCTTCCGACCATCAAGCATTTTATCCGTAC | |
| | | TCCTGATGATGCATGGTTACTCACCACTGCGATCCC | |
| | | CGGAAAAACAGCATTCCAGGTATTAGAAGAATATC | |
| | | CTGATTCAGGTGAAAATATTGTTGATGCGCTGGCAG | |
| | | TGTTCCTGCGCCGGTTGCATTCGATTCCTGTTTGTAA | |
| | | TTGTCCTTTTAACAGCGATCGCGTATTTCGTCTCGCT | |
| | | CAGGCGCAATCACGAATGAATAACGGTTTGGTTGAT | |
| | | GCGAGTGATTTTGATGACGAGCGTAATGGCTGGCCT | |
| | | GTTGAACAAGTCTGGAAAGAAATGCATAAACTTTTG | |
| | | CCATTCTCACCGGATTCAGTCGTCACTCATGGTGAT | |
| | | TTCTCACTTGATAACCTTATTTTTGACGAGGGGAAA | |
| | | TTAATAGGTTGTATTGATGTTGGACGAGTCGGAATC | |
| | | GCAGACCGATACCAGGATCTTGCCATCCTATGGAAC | |
| | | TGCCTCGGTGAGTTTTCTCCTTCATTACAGAAACGG | |
| | | CTTTTTCAAAAATATGGTATTGATAATCCTGATATG | |
| | | AATAAATTGCAGTTTCATTTGATGCTCGATGAGTTT | |
| | | TTCTAATCTCATGACCAAAATCCCTTAACGTGAGTT | |
| | | TTCGTTCCACTGAGCGTCAGACCCCGTAGAAAAGAT | |
| | | CAAAGGATCTTCTTGAGATCCTTTTTTTCTGCGCGTA | |
| | | ATCTGCTGCTTGCAAACAAAAAAACCACCGCTACCA | |
| | | GCGGTGGTTTGTTTGCCGGATCAAGAGCTACCAACT | |
| | | CTTTTTCCGAAGGTAACTGGCTTCAGCAGAGCGCAG | |
| | | ATACCAAATACTGTCCTTCTAGTGTAGCCGTAGTTA | |
| | | GGCCACCACTTCAAGAACTCTGTAGCACCGCCTACA | |
| | | TACCTCGCTCTGCTAATCCTGTTACCAGTGGCTGCT | |
| | | GCCAGTGGCGATAAGTCGTGTCTTACCGGGTTGGAC | |
| | | TCAAGACGATAGTTACCGGATAAGGCGCAGCGGTC | |
| | | GGGCTGAACGGGGGGTTCGTGCACACAGCCCAGCT | |
| | | TGGAGCGAACGACCTACACCGAACTGAGATACCTA | |
| | | CAGCGTGAGCTATGAGAAAGCGCCACGCTTCCCGA | |
| | | AGGGAGAAAGGCGGACAGGTATCCGGTAAGCGGCA | |
| | | GGGTCGGAACAGGAGAGCGCACGAGGGAGCTTCCA | |
| | | GGGGGAAACGCCTGGTATCTTTATAGTCCTGTCGGG | |
| | | TTTCGCCACCTCTGACTTGAGCGTCGATTTTTGTGAT | |
| | | GCTCGTCAGGGGGGCGGAGCCTATGGAAAAACGCC | |
| | | AGCAACGCGGCCTTTTTACGGTTCCTGGCCTTTTGCT | |
| | | GGCCTTTTGCTCACATGTGCGGCCGCACGCGTcatattta | |
| | | tggggtatatgtgaatatttattaca | |
| GPR87 | Nucleic Acid | ATGGCCTTACCAGTGACCGCCTTGCTCCTGCCGCTG | 159 |
| CAR | Sequence | GCCTTGCTGCTCCACGCCGCCAGGCCGGACGTGGTC | |
| | | ATGACCCAGACCCCTCTGAGCCTGCCCGTGAGCCTG | |
| | | GGCGACCAGGCTTCCATCTCCTGCAGAAGCTCCCAG | |
| | | AGCCTGGTGCACAGCAGCGGCAACACCTACCTGCA | |
| | | CTGGTACCTGCAGAAGCCCGGCCAGAGCCCCAAGC | |
| | | TGCTGATCTACAAGGTGTCCAACAGATTTTCCGGCG | |
| | | TGCCCGACAGATTTTCCGGATCCGGCAGCGGCACCG | |
| | | ACTTCACACTGAAGATCAGCAGAGTGGAGGCCGAG | |
| | | GACCTGGGCGTGTACTTCTGTAGCCAGAGCACACAC | |
| | | GTGCCTTACACCTTTGGCGGCGGCACAAAGCTGGAG | |
| | | ATCAAGGGCGGCGGCGGCTCCGGAGGAGGAGGAAG | |
| | | CGGAGGAGGCGGCTCCGAGGTGCAGCTGCAGCAGT | |
| | | CCGGCCCTGACCTGGTGAAGCCTGGCGCCTCCATGA | |
| | | AGATCAGCTGCAAGGCCAGCGGCTACTCCTTCACCG | |
| | | ATTACACCATGCACTGGGTGAAGCAGAGCCACGGC | |
| | | AAGAATTTCGAGTGGATCGGCCTGATCAATCCCTAC | |
| | | AATGACGGCACCACCTACAATCAGAAGTTCAAGGG | |
| | | CAAGGCCACACTGACAGTGGATAAGAGCAGCTCCA | |
| | | CAGCCTACATGGAGCTGCTGAGCCTGACAAGCGAG | |
| | | GATTCCGCCGTGTACTACTGCGCCTCCCTGGACTAC | |
| | | TGGGGCCAGGGCACCTCCGTGACCGTGTCCTCCACC | |
| | | ACTACCCCAGCACCGAGGCCACCCACCCCGGCTCCT | |
| | | ACCATCGCCTCCCAGCCTCTGTCCCTGCGTCCGGAG | |
| | | GCATGTAGACCCGCAGCTGGTGGGGCCGTGCATAC | |
| | | CCGGGGTCTTGACTTCGCCTGCGATTTCTGGGTGCT | |
| | | GGTCGTTGTGGGCGGCGTGCTGGCCTGCTACAGCCT | |
| | | GCTGGTGACAGTGGCCTTCATCATCTTTTGGGTGAG | |
| | | GAGCAAGCGGAGCAGACTGCTGCACAGCGACTACA | |
| | | TGAACATGACCCCCCGGAGGCCTGGCCCCACCCGG | |
| | | AAGCACTACCAGCCCTACGCCCCTCCCAGGGATTTC | |
| | | GCCGCCTACCGGAGCCGCGTGAAATTCAGCCGCAG | |
| | | CGCAGATGCTCCAGCCTACAAGCAGGGGCAGAACC | |
| | | AGCTCTACAACGAACTCAATCTTGGTCGGAGAGAG | |
| | | GAGTACGACGTGCTGGACAAGCGGAGAGGACGGGA | |
| | | CCCAGAAATGGGCGGGAAGCCGCGCAGAAAGAATC | |
| | | CCCAAGAGGGCCTGTACAACGAGCTCCAAAAGGAT | |
| | | AAGATGGCAGAAGCCTATAGCGAGATTGGTATGAA | |
| | | AGGGGAACGCAGAAGAGGCAAAGGCCACGACGGA | |
| | | CTGTACCAGGGACTCAGCACCGCCACCAAGGACAC | |
| | | CTATGACGCTCTTCACATGCAGGCCCTGCCGCCTCG | |
| | | G | |
| signal | Nucleic Acid | ATGGCCTTACCAGTGACCGCCTTGCTCCTGCCGCTG | 160 |
| peptide | Sequence | GCCTTGCTGCTCCACGCCGCCAGGCCG | |
| CD8 Hinge | Nucleic Acid | ACCACTACCCCAGCACCGAGGCCACCCACCCCGGCT | 161 |
| | Sequence | CCTACCATCGCCTCCCAGCCTCTGTCCCTGCGTCCG | |
| | | GAGGCATGTAGACCCGCAGCTGGTGGGGCCGTGCA | |
| | | TACCCGGGGTCTTGACTTCGCCTGCGAT | |
| CD28 TM | Nucleic Acid | TTCTGGGTGCTGGTCGTTGTGGGCGGCGTGCTGGCC | 162 |
| | Sequence | TGCTACAGCCTGCTGGTGACAGTGGCCTTCATCATC | |
| | | TTTTGGGTGAGGAGCAAGCGGAGCAGACTGCTGCA | |
| | | CAGCGACTACATGAACATGACCCCCCGGAGGCCTG | |
| | | GCCCCACCCGGAAGCACTACCAGCCCTACGCCCCTC | |
| | | CCAGGGATTTCGCCGCCTACCGGAGC | |
| CD3z | Nucleic Acid | CGCGTGAAATTCAGCCGCAGCGCAGATGCTCCAGC | 163 |
| | Sequence | CTACAAGCAGGGGCAGAACCAGCTCTACAACGAAC | |
| | | TCAATCTTGGTCGGAGAGAGGAGTACGACGTGCTG | |
| | | GACAAGCGGAGAGGACGGGACCCAGAAATGGGCG | |
| | | GGAAGCCGCGCAGAAAGAATCCCCAAGAGGGCCTG | |
| | | TACAACGAGCTCCAAAAGGATAAGATGGCAGAAGC | |
| | | CTATAGCGAGATTGGTATGAAAGGGGAACGCAGAA | |
| | | GAGGCAAAGGCCACGACGGACTGTACCAGGGACTC | |
| | | AGCACCGCCACCAAGGACACCTATGACGCTCTTCAC | |
| | | ATGCAGGCCCTGCCGCCTCGG | |
| GPR87 | Amino acid | MALPVTALLLPLALLLHAARPDVVMTQTPLSLPVSLG | 164 |
| CAR | sequence | DQASISCRSSQSLVHSSGNTYLHWYLQKPGQSPKLLIY | |
| | | KVSNRFSGVPDRFSGSGSGTDFTLKISRVEAEDLGVYF | |
| | | CSQSTHVPYTFGGGTKLEIKGGGGSGGGGSGGGGSEV | |
| | | QLQQSGPDLVKPGASMKISCKASGYSFTDYTMHWVK | |
| | | QSHGKNFEWILINPYNDGTTYNQKFKGKATLTVDKSS | |
| | | STAYMELLSLTSEDSAVYYCASLDYWGQGTSVTVSST | |
| | | TTPAPRPPTPAPTIASQPLSLRPEACRPAAGGAVHTRGL | |
| | | DFACDFWVLVVVGGVLACYSLLVTVAFIIFWVRSKRS | |
| | | RLLHSDYMNMTPRRPGPTRKHYQPYAPPRDFAAYRS | |
| | | RVKFSRSADAPAYKQGQNQLYNELNLGRREEYDVLD | |
| | | KRRGRDPEMGGKPRRKNPQEGLYNELQKDKMAEAY | |
| | | SEIGMKGERRRGKGHDGLYQGLSTATKDTYDALHMQ | |
| | | ALPPR | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An engineered cell, comprising:
a disrupted B2M gene;
an insertion of a polynucleotide encoding SERPINB9 and a polynucleotide encoding a fusion of IL15 and IL15Rα (IL15/IL15Rα) in the disrupted B2M gene;
a disrupted CIITA gene; and
an insertion of a polynucleotide encoding a CAR and a polynucleotide encoding HLA-E in the disrupted CIITA gene, wherein the CAR is an anti-GPC3 CAR or an anti-GPR87 CAR;
wherein the cell expresses SERPINB9, the IL15/IL15Rα fusion protein, HLA-E, and the CAR, and the cell has disrupted expressions of B2M and CIITA.

2. The engineered cell of claim 1, wherein the engineered cell comprises a disrupted FAS gene, wherein the cell has disrupted expression of FAS.

3. The engineered cell of any one of claims 1-2, wherein the engineered cell comprises a disrupted CISH gene.

4. The engineered cell of claim 3, comprising an insertion of a polynucleotide encoding CD64 in the disrupted CISH gene;
and wherein the cell expresses CD64 and has disrupted expression of CISH.

5. An engineered cell, comprising:
a disrupted B2M gene;
an insertion of a polynucleotide encoding SERPINB9 and a polynucleotide encoding a fusion of IL15 and IL15Rα (IL15/IL15Rα) in the disrupted B2M gene;
a disrupted CIITA gene;
an insertion of a polynucleotide encoding an anti-GPR87 CAR and a polynucleotide encoding HLA-E in the disrupted CIITA gene;
a disrupted CISH gene; and
an insertion of a polynucleotide encoding CD64 in the disrupted CISH gene;
wherein the cell expresses SERPINB9, the IL15/IL15Rα fusion protein, HLA-E, the anti-GPR87 CAR, and CD64, and
wherein the cell has a disrupted expressions of B2M, CIITA, and CISH.

6. An engineered cell, comprising:
a disrupted B2M gene;
an insertion of a polynucleotide encoding SERPINB9 and a polynucleotide encoding a fusion of IL15 and IL15Rα (IL15/IL15Rα) in the disrupted B2M gene;
a disrupted CIITA gene;
an insertion of a polynucleotide encoding an anti-GPC3 CAR and a polynucleotide encoding HLA-E in the disrupted CIITA gene;
a disrupted CISH gene;
an insertion of a polynucleotide encoding CD64 in the disrupted CISH gene; and
a disrupted FAS gene;
wherein the cell expresses SERPINB9, the IL15/IL15Rα fusion protein, HLA-E, the anti-GPC3 CAR, and CD64, and
wherein the cell has disrupted expressions of B2M, CIITA, CISH, and FAS.

7. The engineered cell of any one of claims 1-6, wherein the engineered cell comprises a disrupted TGFβR2 gene, wherein the cell has disrupted expression of TGFβR2.

8. The engineered cell of any one of claims 1-7, wherein the polynucleotide encoding SERPINB9 comprises the sequence of SEQ ID NO: 129 and/or the polynucleotide encoding the IL15/IL15Rα fusion protein comprises the sequence of SEQ ID NO: 76.

9. The engineered cell of any one of claims 1-8, wherein the polynucleotide encoding the anti-GPC3 comprises the sequence of SEQ ID NO: 152 and/or anti-GPR87 CAR comprises the sequence of SEQ ID NO: 159.

10. The engineered cell of any one of claims 1-9, wherein the polynucleotide encoding HLA-E comprises the sequence of SEQ ID NO: 51; optionally, the polynucleotide encoding HLA-E comprises a sequence encoding an HLA-E trimer comprising the sequence of SEQ ID NO: 75.

11. The engineered cell of any one of claims 4-10, wherein the polynucleotide encoding CD64 comprises the sequence of SEQ ID NO: 146.

12. The engineered cell of any one of claims 1-11, wherein the engineered cell is a stem cell.

13. The engineered cell of claim 12, wherein the stem cell is an induced pluripotent stem cell (iPSC), a hematopoietic stem cell, an embryonic stem cell, or an adult stem cell.

14. The engineered cell of any one of claims 1-11, wherein the engineered cell is a genome-edited iPSC.

15. The engineered cell of any one of claims 1-11, wherein the engineered cell is a natural killer (NK) cell obtained from a genome-edited iPSC.

16. The engineered cell of any one of claims 1-11, wherein the engineered cell is a differentiated cell or a somatic cell.

17. The engineered cell of any one of claims 1-11, wherein the engineered cell is capable of being differentiated into lineage-restricted progenitor cells or fully differentiated somatic cells.

18. The engineered cell of any one of claims 1-11, wherein the engineered cell is a natural killer (NK) cell.

19. The engineered cell of claim 18, wherein the NK cell has been differentiated from a genome-edited iPSC, wherein the NK cell comprises the genome edits of the genome-edited iPSC, and wherein the NK cell has not been genome-edited after the differentiation.

20. The engineered cell of any one of claims 1-19, wherein the engineered cell is capable of cell expansion in the absence of exogenous IL15 in cell culture media.

21. A population of cells, comprising one or more engineered cells of any one of claims 1-20.

22. A population of cells, comprising lineage-restricted progenitor cells or fully differentiated somatic cells derived from one or more engineered cells of any one of claims 1-20.

23. The population of cells of claim 22, wherein the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells; and the fully differentiated somatic cells are pancreatic beta cells, epithelial cells, endodermal cells, macrophages, hepatocytes, adipocytes, kidney cells, blood cells, cardiomyocytes, or immune system cells.

24. The population of cells of claim 22, wherein the population of cells comprises NK cells, T cells, B cells, or NKT cells.

25. The population of cells of claim 24, wherein the population of cells comprises human NK cells.

26. The population of cells of claim 25, wherein the human NK cells express at least one, two, three, four or five of the markers of CD56, NKp44, NKp46, CD94, NKG2A, KIR2DL4, and a CAR, wherein the CAR is detected by Protein L binding; and optionally wherein the at least one, two, three, four or five markers are expressed in at least 25%, 30%, 40%, 50%, or 75% of the population of cells.

27. The population of cells of any one of claims 25-26, wherein the population of cells comprising human NK cells has at least one of the following characteristics, or any combination thereof: (i) an alloimmune T cell reaction of less than 10% relative to a population of unmodified human NK cells, (ii) cytotoxic activity resulting in killing more than 50% of target cells when the population of cells comprising human NK cells are mixed with the target cells at the ratio of 1:1, and (iii) at least 50% increase in cellular viability relative to a population of unmodified human NK cells.

28. The population of cells of any one of claims 25-27, wherein the population of cells comprising human NK cells has at least one of the following characteristics, or any combination thereof: (i) improved persistency, (ii) improved immune evasiveness, (iii) improved cytotoxic activity, (iv) improved antibody-dependent cellular cytotoxicity (ADCC) activity, and (v) improved anti-tumor activity; wherein the characteristics are improved relative to a population of unmodified human NK cells.

29. The population of cells of any one of claims 25-28, wherein the population of cells comprising human NK cells, when co-cultured in vitro with a population of cancer cells, induce cell death of at least 60%, at least 70%, at least 80%, or at least 90% of the population of cancer cells after about 24 hours of co-culture.

30. The population of cells of any one of claims 25-29, wherein the population of cells comprising human NK cells, when co-cultured in vitro with a population of cancer cells, secrete at least one of interferon gamma (IFNγ), tumor necrosis factor alpha (TNFα), and Granzyme B (GRNB).

31. The population of cells of any one of claims 29-30, wherein the ratio of the human NK cells to cancer cells is 0.1:1 to 4:1.

32. A composition, comprising the population of cells of any one of claims 21-31.

33. The composition of claim 32 for use in treating a subject in need thereof.

34. The composition of claim 32 for use in treating cancer in a subject in need thereof.

35. The composition of claim 34, wherein the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC), pancreatic cancer, melanoma, ovarian cancer, glioblastoma, hepatocellular carcinoma, or cervical cancer.

36. The composition of any one of claims 32-35, wherein the subject is human.

37. A method of obtaining cells for administration to a subject in need thereof, the method comprising:
(a) obtaining or having obtained the population of cells of claim 21, and
(b) maintaining the population of cells for a time and under conditions sufficient for the one or more engineered cells to differentiate into lineage-restricted progenitor cells or fully differentiated somatic cells.

38. A method for treating a subject in need thereof, the method comprising:
(a) obtaining or having obtained the population of cells of claim 22; and
(b) administering the lineage-restricted progenitor cells or fully differentiated somatic cells to the subject.

39. The method of claim 37 or 38, wherein the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells are pancreatic beta cells, epithelial cells, endodermal cells, macrophages, hepatocytes, adipocytes, kidney cells, blood cells, cardiomyocytes, or immune system cells.

40. The method of any one of claims 37-39, wherein the subject has, is suspected of having, or is at risk for a cancer.

41. The method of any one of claims 37-40, wherein the subject is human.

42. An in vitro method for generating an engineered cell, the method comprising delivering to a cell:
an RNP complex comprising an RNA-guided endonuclease and a gRNA targeting a target site in a B2M gene locus or an RNA-guided endonuclease and a gRNA targeting a target site in the B2M gene locus;
a vector comprising a nucleic acid, the nucleic acid comprising: (i) a nucleotide sequence encoding SERPINB9 and a nucleotide sequence encoding a fusion of IL15 and IL15Rα (IL15/IL15Rα); (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii);
an RNP complex comprising an RNA-guided endonuclease and a gRNA targeting a target site in a CIITA gene locus or an RNA-guided endonuclease and a gRNA targeting a target site in the CIITA gene locus; and
a vector comprising a nucleic acid, the nucleic acid comprising: (i) a nucleotide sequence encoding a CAR and a nucleotide sequence encoding an HLA-E trimer, wherein the CAR is an anti-GPC3 CAR or an anti-GPR87 CAR; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii);
wherein the B2M gene locus is cleaved at the target site and the nucleotide sequences encoding SERPINB9 and the IL15/IL15Rα fusion protein are inserted into the B2M gene locus, thereby disrupting the B2M gene, and
wherein the CIITA gene locus is cleaved at the target site and the nucleotide sequences encoding the CAR and the HLA-E trimer are inserted into the CIITA gene locus, thereby disrupting the CIITA gene.

43. The in vitro method of claim 42, wherein the method comprises delivering to the cell:
an RNP complex comprising an RNA-guided endonuclease and a gRNA targeting a target site in a CISH gene locus or an RNA-guided endonuclease and a gRNA targeting a target site in the CISH gene locus, and optionally a vector comprising a nucleic acid, the nucleic acid comprising: (i) a nucleotide sequence encoding CD64; (ii) a nucleotide sequence having sequence homology with a genomic region located left of the target site in the CISH gene locus; and (iii) a nucleotide sequence having sequence homology with a genomic region located right of the target site in the CISH gene locus, wherein (i) is flanked by (ii) and (iii),
wherein the CISH gene locus is cleaved at the target site and the nucleotide sequence encoding CD64 is inserted into the CISH gene locus, thereby disrupting the CISH gene.

44. The in vitro method of any one of claims 42-43, wherein the method comprises delivering to the cell:
an RNP complex comprising an RNA-guided endonuclease and a gRNA targeting a target site in a FAS gene locus or an RNA-guided endonuclease and a gRNA targeting a target site in the FAS gene locus, wherein the FAS gene locus is cleaved at the target site, thereby disrupting the FAS gene.

45. The in vitro method of any one of claims 42-44, wherein the method comprises delivering to the cell:
an RNP complex comprising an RNA-guided endonuclease and a gRNA targeting a target site in a TGFβR2 gene locus or an RNA-guided endonuclease and a gRNA targeting a target site in the TGFβR2 gene locus, wherein the TGFβR2 gene locus is cleaved at the target site, thereby disrupting the TGFβR2 gene.

46. The in vitro method of any one of claims 42-45, wherein the RNP complex comprising the RNA-guided endonuclease and the gRNA targeting a target site in the CIITA gene locus or the RNA-guided endonuclease and the gRNA targeting a target site in the CIITA gene locus; and the vector comprising the nucleic acid comprising: (i) the nucleotide sequence encoding the CAR and the nucleotide sequence encoding the HLA-E trimer; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and iii);
are delivered to the cell after the nucleotide sequences encoding SERPINB9 and the IL15/IL15Rα fusion protein are inserted into the B2M gene locus.

47. The in vitro method of any one of claims 43-46, wherein the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii);
are delivered to the cell after the nucleotide sequences encoding SERPINB9 and the IL15/IL15Rα fusion protein are inserted into the B2M gene locus and after the nucleotide sequences encoding the CAR and the HLA-E trimer are inserted into the CIITA gene locus.

48. The in vitro method of any one of claims 44-47, wherein the RNP complex comprising the RNA-guided endonuclease and the gRNA targeting the target site in the FAS gene locus or the RNA-guided endonuclease and the gRNA targeting the target site in the FAS gene locus,
are delivered to the cell after the nucleotide sequences encoding SERPINB9 and the IL15/IL15Rα fusion protein are inserted into the B2M gene locus, the nucleotide sequences encoding the CAR and the HLA-E trimer are inserted into the CIITA gene locus, and after the nucleotide sequence encoding CD64 is inserted into the CISH gene locus.

49. The method of any one of claims 45-47, wherein the RNP complex comprising the RNA-guided endonuclease and the gRNA targeting the target site in the TGFβR2 gene locus or the RNA-guided endonuclease and the gRNA targeting the target site in the TGFβR2 gene locus,
are delivered to the cell after the nucleotide sequences encoding SERPINB9 and the IL15/IL15Rα fusion protein are inserted into the B2M gene locus, after the nucleotide sequences encoding the CAR and the HLA-E trimer are inserted into the CIITA gene locus, and after the nucleotide sequence encoding CD64 is inserted into the CISH gene locus; and
wherein the RNP complex comprising the RNA-guided endonuclease and the gRNA targeting a target site in the FAS gene locus or the RNA-guided endonuclease and the gRNA targeting a target site in the FAS gene locus;
are delivered to the cell after the nucleotide sequence encoding CD64 is inserted into the CISH gene locus, and after the TGFβR2 gene locus is disrupted.

50. The in vitro method of claim 43 or 44, wherein the RNP complex comprising the RNA-guided endonuclease and the gRNA targeting the target site in the CISH gene locus or the RNA-guided endonuclease and the gRNA targeting the target site in the CISH gene locus and/or the vector comprising the nucleic acid comprising: (i) the nucleotide sequence encoding CD64; (ii) the nucleotide sequence having sequence homology with a genomic region located left of the target site in the CISH gene locus; and (iii) the nucleotide sequence having sequence homology with a genomic region located right of the target site in the CISH gene locus, wherein (i) is flanked by (ii) and (iii);
are delivered to the cell after the nucleotide sequences encoding SERPINB9 and the IL15/IL15Rα fusion protein are inserted into the B2M gene locus.

51. The in vitro method of claim 44 or 50, wherein the RNP complex comprising the RNA-guided endonuclease and the gRNA targeting the target site in the FAS gene locus or the RNA-guided endonuclease and the gRNA targeting the target site in the FAS gene locus,
are delivered to the cell after the nucleotide sequences encoding SERPINB9 and the IL15/IL15Rα fusion protein are inserted into the B2M gene locus and after the nucleotide sequence encoding CD64 is inserted into the CISH gene locus.

52. The in vitro method of claim 44 or 51, wherein the RNP complex comprising the RNA-guided endonuclease and the gRNA targeting the target site in the CIITA gene locus or the RNA-guided endonuclease and the gRNA targeting the target site in the CIITA gene locus and the vector comprising the nucleic acid comprising: (i) the nucleotide sequence encoding the CAR and the nucleotide sequence encoding the HLA-E trimer; (ii) the nucleotide sequence having sequence homology with a genomic region located left of the target site in the CIITA gene locus; and (iii) the nucleotide sequence having sequence homology with a genomic region located right of the target site in the CIITA gene locus, wherein (i) is flanked by (ii) and (iii);
are delivered to the cell after the nucleotide sequences encoding SERPINB9 and the IL15/IL15Rα fusion protein are inserted into the B2M gene locus, after the nucleotide sequence encoding CD64 is inserted into the CISH gene locus, and after the FAS gene locus is disrupted.

53. The in vitro method of any one of claims 42-52, wherein the gRNA targeting the target site in the B2M gene locus comprises a spacer sequence of any one of SEQ ID NOs: 180 and 187-188.

54. The in vitro method of any one of claims 42-52, wherein the gRNA targeting the target site in the B2M gene locus comprises a spacer sequence of SEQ ID NO: 180.

55. The in vitro method of any one of claims 42-54, wherein the gRNA targeting the target site in the CIITA gene locus comprises a spacer sequence of any one of SEQ ID NOs: 175-179.

56. The in vitro method of any one of claims 42-54, wherein the gRNA targeting the target site in the CIITA gene locus comprises a spacer sequence of SEQ ID NO: 175.

57. The in vitro method of any one of claims 43-56, wherein the gRNA targeting the target site in the CISH gene locus comprises a spacer sequence of any one of SEQ ID NOs: 190-201.

58. The in vitro method of any one of claims 43-56, wherein the gRNA targeting the target site in the CISH gene locus comprises a spacer sequence of SEQ ID NO: 191.

59. The in vitro method of any one of claims 44-58, wherein the gRNA targeting the target site in the FAS gene locus comprises a spacer sequence of any one of SEQ ID NOs: 181-186 and 189.

60. The in vitro method of any one of claims 44-58, wherein the gRNA targeting the target site in the FAS gene locus comprises a spacer sequence of SEQ ID NO: 181 or SEQ ID NO: 182.

61. The in vitro method of any one of claims 45-60, wherein the gRNA targeting the target site in the TGFβR2 gene locus comprises a spacer sequence of SEQ ID NO: 211.

62. The in vitro method of any one of claims 42-61, wherein the nucleotide sequence having sequence homology with a genomic region located left of the target site in the B2M gene locus comprises the sequence of SEQ ID NO: 36 and the nucleotide sequence having sequence homology with a genomic region located right of the target site in the B2M gene locus comprises the sequence of SEQ ID NO: 54.

63. The in vitro method of any one of claims 42-62, wherein the nucleotide sequence having sequence homology with a genomic region located left of the target site in the CIITA gene locus comprises the sequence of SEQ ID NO: 22 and the nucleotide sequence having sequence homology with a genomic region located right of the target site in the CIITA gene locus comprises the sequence of SEQ ID NO: 32.

64. The in vitro method of any one of claims 43-63, wherein the nucleotide sequence having sequence homology with a genomic region located left of the target site in the CISH gene locus comprises the sequence of SEQ ID NO: 145 and nucleotide sequence having sequence homology with a genomic region located right of the target site in the CISH gene locus comprises the sequence of SEQ ID NO: 148.

65. The in vitro method of any one of claims 42-64, wherein the nucleotide sequence encoding SERPINB9 comprises the sequence of SEQ ID NO: 129 and the nucleotide sequence encoding the IL15/IL15Rα fusion protein comprises the sequence of SEQ ID NO: 76.

66. The in vitro method of any one of claims 42-65, wherein the nucleotide sequence encoding the anti-GPC3 CAR comprises the sequence of SEQ ID NO: 152.

67. The in vitro method of any one of claims 42-66, wherein the nucleotide sequence encoding the anti-GPR87 CAR comprises the sequence of SEQ ID NO: 159.

68. The in vitro method of any one of claims 42-67, wherein the nucleotide sequence encoding the HLA-E trimer comprises the sequence of SEQ ID NO: 75.

69. The in vitro method of any one of claims 43-68, wherein the nucleotide sequence encoding CD64 comprises the sequence of SEQ ID NO: 146.

70. The in vitro method of any one of claims 42-69, wherein the cell is a stem cell.

71. The in vitro method of claim 70, wherein the stem cell is an embryonic stem cell, an adult stem cell, an induced pluripotent stem cell, or a hematopoietic stem cell.

72. The in vitro method of claim 70 or 71, wherein the stem cell is a human stem cell.

73. A population of cells comprising one or more engineered cells generated by the method of any one of claims 42-72.

74. The population of cells of claim 73, wherein the population of cells is maintained for a time and under conditions sufficient for at least a portion of the one or more engineered cells to undergo differentiation.

75. The population of cells of claim 73 or 74, for use in treating a subject in need thereof; and optionally wherein the subject is a human who has, is suspected of having, or is at risk for a cancer.

76. A method, comprising administering to a subject in need thereof the population of cells of claim 73 or 74.

77. A method for treating a subject in need thereof, the method comprising:
(a) obtaining or having obtained the population of cells of claim 74 following differentiation into lineage-restricted progenitor cells or fully differentiated somatic cells; and
(b) administering the lineage-restricted progenitor cells or fully differentiated somatic cells to the subject.

78. A method of obtaining cells for administration to a subject in need thereof, the method comprising:
(a) obtaining or having obtained the population of cells of claim 73; and
(b) maintaining the engineered cells for a time and under conditions sufficient for the one or more engineered cells to differentiate into lineage-restricted progenitor cells or fully differentiated somatic cells.

79. The method of claim 78 or 79, wherein the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, definitive endoderm, hepatoblasts, pancreatic endoderm progenitors, pancreatic endocrine progenitors, mesenchymal progenitor cells, muscle progenitor cells, blast cells, or neural progenitor cells; and the fully differentiated somatic cells are hepatocytes, pancreatic beta cells, epithelial cells, endodermal cells, macrophages, hepatocytes, adipocytes, kidney cells, blood cells, cardiomyocytes, or immune system cells.

80. The method of any one of claims 78-80, wherein the fully differentiated somatic cells are NK cells.

81. The method of any one of claims 77-81, wherein the subject is a human who has, is suspected of having, or is at risk for a cancer.

82. The method of claim 82, comprising administering the NK cells to the subject who has, is suspected of having or is at risk for a cancer, thereby inhibiting the progression of the cancer.

83. A method of treating cancer, the method comprising administrating NK cells obtained by the method of claim 81 to a subject with cancer, thereby inhibiting progression of the cancer.

84. The method of any one of claims 82-84, wherein the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, pancreatic cancer, liver cancer, melanoma, ovarian cancer, glioblastoma, or cervical cancer.

85. The method of claim 85, wherein the liver cancer is hepatocellular carcinoma.

86. The method of claim 85, wherein the lung cancer is non-small cell lung cancer.

87. The method of any one of claims 83-87, comprising administering about 1×10² to 1×10¹⁰ per gram to the subject; and optionally about 1×10⁶ NK cells per gram to the subject.

88. The method of any one of claims 83-88, wherein the NK cells are administered to the subject more than once; optionally, the NK cells are administered to the subject at least three times.

89. The method of any one of claims 83-89, wherein the NK cells are administered to the subject in a cycle of at least 7 days.

90. The method of any one of claims 83-90, wherein the NK cells are administered to the subject one, two, or three times in a week.

91. The method of any one of claims 83-91, wherein inhibiting progression of the cancer comprises inhibition of growth of one or more tumors in the subject and/or reducing the number of cancer cells detected in the subject; relative to an untreated subject.

92. The method of any one of claims 83-92, wherein inhibiting progression of the cancer comprises inhibition of growth of one or more tumors in the subject and/or reducing the number of cancer cells detected in the subject; relative to the subject prior to administration of the NK cells.

93. The method of claim 92 or 93, wherein the number of cancer cells detected in the subject increases by no more than 0.5-fold after administration of the NK cells, following one or more cycles of treatment.

94. The method of claim 92 or 93, wherein the growth of at least one of the one or more tumors in the subject is inhibited by at least about 70% following one or more cycles of treatment.

95. The method of any one of claims 83-95, wherein the subject is tumor-free following one or more cycles of treatment.

96. The method of any one of claims 83-96, wherein the NK cells persist in the subject for at least one week following administration.

97. The method of any one of claims 83-97, wherein the number of NK cells detected in the subject decreases by less than 25% one week after administration or by less than 50% two weeks after administration.

98. The method of any one of claims 98-98, wherein the NK cells are localized to the site of the cancer following administration.

99. A gRNA or sgRNA for targeting a TGFβR2 gene locus, wherein the gRNA or sgRNA comprises a spacer sequence of SEQ ID NO: 211.


SEQUENCE LISTING (211 sequences)

SEQ ID NO: 1 - DNA, 20 bp, synthetic construct
  source 1..20: mol_type = other DNA
ggtcgcggcgccagcacgaa

SEQ ID NO: 2 - DNA, 20 bp, synthetic construct
  source 1..20: mol_type = other DNA
ccgaagcccgggtcatccgg

SEQ ID NO: 3 - DNA, 20 bp, synthetic construct
  source 1..20: mol_type = other DNA
ccgcgacctccggatgaccc

SEQ ID NO: 4 - DNA, 20 bp, synthetic construct
  source 1..20: mol_type = other DNA
cgtgctggcgccgcgacctc

SEQ ID NO: 5 - DNA, 20 bp, synthetic construct
  source 1..20: mol_type = other DNA
cgaaaggaaccacgctggtc

SEQ ID NO: 6 - DNA, 20 bp, synthetic construct
  source 1..20: mol_type = other DNA
cagcgtggttcctttcgtgc

SEQ ID NO: 7 - DNA, 20 bp, synthetic construct
  source 1..20: mol_type = other DNA
gccgcgacctccggatgacc

SEQ ID NO: 8 - DNA, 20 bp, synthetic construct
  source 1..20: mol_type = other DNA
gaaccacgctggtcaggaat

SEQ ID NO: 9 - DNA, 20 bp, synthetic construct
  source 1..20: mol_type = other DNA
cagcacgaaaggaaccacgc

SEQ ID NO: 10 - DNA, 20 bp, synthetic construct
  source 1..20: mol_type = other DNA
gtagcggggccgggaacatg

SEQ ID NO: 11 - DNA, 20 bp, synthetic construct
  source 1..20: mol_type = other DNA
agaatcttcccagtaggcgg

SEQ ID NO: 12 - DNA, 20 bp, synthetic construct
  source 1..20: mol_type = other DNA
ctcaggcgctcagtcactac

SEQ ID NO: 13 - DNA, 20 bp, synthetic construct
  source 1..20: mol_type = other DNA
ggtccatctggtcatagaag

SEQ ID NO: 14 - DNA, 20 bp, synthetic construct
  source 1..20: mol_type = other DNA
gctccaggtagccaccttct

SEQ ID NO: 15 - DNA, 20 bp, synthetic construct
  source 1..20: mol_type = other DNA
taggggccccaactccatgg

SEQ ID NO: 16 - DNA, 20 bp, synthetic construct
  source 1..20: mol_type = other DNA
ggcttatgccaatatcggtg

SEQ ID NO: 17 - DNA, 20 bp, synthetic construct
  source 1..20: mol_type = other DNA
aggtgatgaagagaccaggg

SEQ ID NO: 18 - DNA, 22 bp, synthetic construct
  source 1..22: mol_type = other DNA
tcctgactctctggtgtgagat

SEQ ID NO: 19 - DNA, 19 bp, synthetic construct
  source 1..19: mol_type = other DNA
cagagagcgtcccacagac

SEQ ID NO: 20 - DNA, 86 bp, synthetic construct
  source 1..86: mol_type = other DNA
gccaccatggagttggggcccctagaaggtggctacctggagcttcttaacagcgatgctgaccccctgtgcctctaccacttcta

SEQ ID NO: 21 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 22 - DNA, 800 bp, synthetic construct
  source 1..800: mol_type = other DNA
catatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgt

SEQ ID NO: 23 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 24 - DNA, 276 bp, synthetic construct
  source 1..276: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 25 - DNA, 1009 bp, synthetic construct
  source 1..1009: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 26 - DNA, 63 bp, synthetic construct
  source 1..63: mol_type = other DNA
atggcgcttccggtgacagcactgctcctccccttggcgctgttgctccacgcagcaaggccg

SEQ ID NO: 27 - DNA, 735 bp, synthetic construct
  source 1..735: mol_type = other DNA
caggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaa

SEQ ID NO: 28 - DNA, 264 bp, synthetic construct
  source 1..264: mol_type = other DNA
agtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 29 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - DNA, 336 bp, synthetic construct
  source 1..336: mol_type = other DNA
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 31 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 32 - DNA, 795 bp, synthetic construct
  source 1..795: mol_type = other DNA
tgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgc

SEQ ID NO: 33 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 20 bp, synthetic construct
  source 1..20: mol_type = other DNA
ggccgagatgtctcgctccg

SEQ ID NO: 35 - DNA, 20 bp, synthetic construct
  source 1..20: mol_type = other DNA
ggattgctcaacaaccatgc

SEQ ID NO: 36 - DNA, 800 bp, synthetic construct
  source 1..800: mol_type = other DNA
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagct

SEQ ID NO: 37 - DNA, 20 bp, synthetic construct
  source 1..20: mol_type = other DNA
gattgctcaacaaccatgct

SEQ ID NO: 38 - DNA, 20 bp, synthetic construct
  source 1..20: mol_type = other DNA
gtgactgacatcaactccaa

SEQ ID NO: 39 - DNA, 20 bp, synthetic construct
  source 1..20: mol_type = other DNA
cacttgggcattaacacttt

SEQ ID NO: 40 - DNA, 54 bp, synthetic construct
  source 1..54: mol_type = other DNA
atggactggacctggatcctgttcctggtggccgccgccaccagggtgcacagc

SEQ ID NO: 41 - DNA, 399 bp, synthetic construct
  source 1..399: mol_type = other DNA
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 42 - DNA, 78 bp, synthetic construct
  source 1..78: mol_type = other DNA
agcggcggcggcagcggcggcggcggcagcggcggcggcggcagcggcggcggcggcagcggcggcggcagcctgcag

SEQ ID NO: 43 - DNA, 711 bp, synthetic construct
  source 1..711: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 44 - skipped

SEQ ID NO: 45 - DNA, 57 bp, synthetic construct
  source 1..57: mol_type = other DNA
gctactaacttcagcctgctgaagcaggctggagacgtggaggagaaccctggacct

SEQ ID NO: 46 - DNA, 60 bp, synthetic construct
  source 1..60: mol_type = other DNA
atgtctcgctccgttgccttagctgtgctcgcgctactctctctttctggattagaggct

SEQ ID NO: 47 - DNA, 27 bp, synthetic construct
  source 1..27: mol_type = other DNA
gtcatggcgccccgaaccctcttcctg

SEQ ID NO: 48 - DNA, 45 bp, synthetic construct
  source 1..45: mol_type = other DNA
ggtggaggcggttcaggcggaggtggctctggcggtggcggatcg

SEQ ID NO: 49 - DNA, 297 bp, synthetic construct
  source 1..297: mol_type = other DNA
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 50 - DNA, 60 bp, synthetic construct
  source 1..60: mol_type = other DNA
ggtggtggtggttctggtggtggtggttctggcggcggcggctccggtggtggtggatcc

SEQ ID NO: 51 - DNA, 1011 bp, synthetic construct
  source 1..1011: mol_type = other DNA
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 52 - skipped

SEQ ID NO: 53 - DNA, 20 bp, synthetic construct
  source 1..20: mol_type = other DNA
ttggaaggcctgcatcatga

SEQ ID NO: 54 - DNA, 800 bp, synthetic construct
  source 1..800: mol_type = other DNA
ccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 55 - DNA, 20 bp, synthetic construct
  source 1..20: mol_type = other DNA
actccaagggattggaattg

SEQ ID NO: 56 - DNA, 20 bp, synthetic construct
  source 1..20: mol_type = other DNA
cagacagcaaactcacccag

SEQ ID NO: 57 - DNA, 20 bp, synthetic construct
  source 1..20: mol_type = other DNA
aaactttgtcccgaccctcc

SEQ ID NO: 58 - DNA, 52 bp, synthetic construct
  source 1..52: mol_type = other DNA
aaaagatctgtggactccaccaccacgaaatggcggcaccttatttatggtc

SEQ ID NO: 59 - DNA, 59 bp, synthetic construct
  source 1..59: mol_type = other DNA
gctctggagaatctcacgcagaaggcaggcgtttttcttaaaaaaaaatgcacgaatta

SEQ ID NO: 60 - DNA, 42 bp, synthetic construct
  source 1..42: mol_type = other DNA
aggattgggaagacaatagcaggcatgctggggatgcggtgg

SEQ ID NO: 61 - DNA, 58 bp, synthetic construct
  source 1..58: mol_type = other DNA
gctctggagaatctcacgcagaaggcaggctttttcttaaaaaaaaatgcacgaatta

SEQ ID NO: 62 - DNA, 54 bp, synthetic construct
  source 1..54: mol_type = other DNA
gccccacccctcctactttatgtctccatggatttgcctgttttggtcatttca

SEQ ID NO: 63 - DNA, 58 bp, synthetic construct
  source 1..58: mol_type = other DNA
ctctaatgcaaacttgggtaggtcgtttcacctctctaaacctcaatttcctcatttg

SEQ ID NO: 64 - DNA, 50 bp, synthetic construct
  source 1..50: mol_type = other DNA
gagtgaagttttcccgaagcgcagacgctccggcatatcagcaaggacag

SEQ ID NO: 65 - DNA, 58 bp, synthetic construct; identical to SEQ ID NO: 63
  source 1..58: mol_type = other DNA

SEQ ID NO: 66 - DNA, 7788 bp, synthetic construct
  source 1..7788: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataatccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 67 - DNA, 9077 bp, synthetic construct
  source 1..9077: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 68 - protein, 22 aa, synthetic construct
MLLLVTSLLLCELPHPAFLLIP

SEQ ID NO: 69 - protein, 21 aa, synthetic construct
MALPVTALLLPLALLLHAARP

SEQ ID NO: 70 - DNA, 1524 bp, synthetic construct
  source 1..1524: mol_type = other DNA
atggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 71 - DNA, 735 bp, synthetic construct; identical to SEQ ID NO: 27
  source 1..735: mol_type = other DNA

SEQ ID NO: 72 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 73 - protein, 23 aa, synthetic construct
IYIWAPLAGTCGVLLLSLVITLY

SEQ ID NO: 74 - protein, 508 aa, synthetic construct
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1500 bp, synthetic construct
  source 1..1500: mol_type = other DNA
atgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 76 - DNA, 1251 bp, synthetic construct
  source 1..1251: mol_type = other DNA
atggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggaagcgga

SEQ ID NO: 77 - DNA, 2808 bp, synthetic construct
  source 1..2808: mol_type = other DNA
atggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 78 - DNA, 20 bp, synthetic construct
  source 1..20: mol_type = other DNA
gctactctctctttctggcc

SEQ ID NO: 79 - DNA, 20 bp, synthetic construct
  source 1..20: mol_type = other DNA
cgcgagcacagctaaggcca

SEQ ID NO: 80 - DNA, 20 bp, synthetic construct
  source 1..20: mol_type = other DNA
ctagggactgcacagtcaat

SEQ ID NO: 81 - DNA, 20 bp, synthetic construct
  source 1..20: mol_type = other DNA
tcgccgctgccgcggggaca

SEQ ID NO: 82 - DNA, 20 bp, synthetic construct
  source 1..20: mol_type = other DNA
gacatggtcctctgcgttca

SEQ ID NO: 83 - DNA, 20 bp, synthetic construct
  source 1..20: mol_type = other DNA
gtccgctccacagccagcaa

SEQ ID NO: 84 - DNA, 20 bp, synthetic construct
  source 1..20: mol_type = other DNA
gttccagggacggggcccac

SEQ ID NO: 85 - DNA, 20 bp, synthetic construct
  source 1..20: mol_type = other DNA
tcgggcctcgctggccgtaa

SEQ ID NO: 86 - DNA, 20 bp, synthetic construct
  source 1..20: mol_type = other DNA
cgtactaagaacgtgccttc

SEQ ID NO: 87 - DNA, 20 bp, synthetic construct
  source 1..20: mol_type = other DNA
gggttccattacggccagcg

SEQ ID NO: 88 - DNA, 20 bp, synthetic construct
  source 1..20: mol_type = other DNA
caggtgttgtcgggcctcgc

SEQ ID NO: 89 - DNA, 20 bp, synthetic construct
  source 1..20: mol_type = other DNA
tactcaatgcgtacattggt

SEQ ID NO: 90 - DNA, 20 bp, synthetic construct
  source 1..20: mol_type = other DNA
aaggctgaccacatccggaa

SEQ ID NO: 91 - DNA, 20 bp, synthetic construct
  source 1..20: mol_type = other DNA
tacattggtggggccacgag

SEQ ID NO: 92 - DNA, 20 bp, synthetic construct
  source 1..20: mol_type = other DNA
ctgtcagtgaaaaccactcg

SEQ ID NO: 93 - DNA, 20 bp, synthetic construct
  source 1..20: mol_type = other DNA
ggtcatcgatgggagcaacg

SEQ ID NO: 94 - DNA, 20 bp, synthetic construct
  source 1..20: mol_type = other DNA
caccaccccgcgggactaga

SEQ ID NO: 95 - DNA, 20 bp, synthetic construct
  source 1..20: mol_type = other DNA
ggtctggcgctcccgctcgg

SEQ ID NO: 96 - DNA, 20 bp, synthetic construct
  source 1..20: mol_type = other DNA
ccaccaccccgcgggactag

SEQ ID NO: 97 - DNA, 20 bp, synthetic construct
  source 1..20: mol_type = other DNA
ttaggggtgccaccaccccg

SEQ ID NO: 98 - DNA, 20 bp, synthetic construct
  source 1..20: mol_type = other DNA
ttcacaccatcacgacgcgt

SEQ ID NO: 99 - DNA, 20 bp, synthetic construct
  source 1..20: mol_type = other DNA
acaccatcacgacgcgtggg

SEQ ID NO: 100 - DNA, 20 bp, synthetic construct
  source 1..20: mol_type = other DNA
ctacgagtctgacgggatcg

SEQ ID NO: 101 - DNA, 20 bp, synthetic construct
  source 1..20: mol_type = other DNA
acgacgcgtgggtggcaagc

SEQ ID NO: 102 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 103 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 104 - protein, 107 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIK

SEQ ID NO: 105 - protein, 112 aa, synthetic construct
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 106 - DNA, 729 bp, synthetic construct
  source 1..729: mol_type = other DNA
cagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaag

SEQ ID NO: 107 - DNA, 120 bp, synthetic construct
  source 1..120: mol_type = other DNA
agcaagagaagcagactgctgcacagcgactacatgaacatgacccccagaagacccggccccaccagaaagcactaccagccctacgccccccccagagacttcgccgcctacagaagc

SEQ ID NO: 108 - DNA, 1449 bp, synthetic construct
  source 1..1449: mol_type = other DNA
cagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 109 - protein, 483 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAGGGGSGGGGSGGGGSDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - DNA, 11265 bp, synthetic construct
  source 1..11265: mol_type = other DNA
agaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatctt

SEQ ID NO: 111 - DNA, 702 bp, synthetic construct
  source 1..702: mol_type = other DNA
caggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaag

SEQ ID NO: 112 - DNA, 1428 bp, synthetic construct
  source 1..1428: mol_type = other DNA
caggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 113 - protein, 476 aa, synthetic construct
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSGGGGSGGGGSGGGGSDIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - DNA, 11235 bp, synthetic construct
  source 1..11235: mol_type = other DNA
atgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattacatgcatagaaggtataatgatc

SEQ ID NO: 115 - DNA, 696 bp, synthetic construct
  source 1..696: mol_type = other DNA
gacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtg

SEQ ID NO: 116 - DNA, 1422 bp, synthetic construct
  source 1..1422: mol_type = other DNA
gacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 117 - protein, 474 aa, synthetic construct
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGGGGSGGGGSGGGGSQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 11238 bp, synthetic construct
  source 1..11238: mol_type = other DNA
tgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattaca

SEQ ID NO: 119 - DNA, 3015 bp, synthetic construct
  source 1..3015: mol_type = other DNA
cagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 120 - DNA, 2985 bp, synthetic construct
  source 1..2985: mol_type = other DNA
caggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 121 - DNA, 2988 bp, synthetic construct
  source 1..2988: mol_type = other DNA
gacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 122 - protein, 88 aa, synthetic construct
SAAAFVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 123 - protein, 40 aa, synthetic construct
SKRSRLLHSDYMNMTPRRPGPTRKHYQPYAPPRDFAAYRS

SEQ ID NO: 124 - protein, 42 aa, synthetic construct
KRGRKKLLYIFKQPFMRPVQTTQEEDGCSCRFPEEEEGGCEL

SEQ ID NO: 125 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - DNA, 264 bp, synthetic construct
  source 1..264: mol_type = other DNA
agcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacaga

SEQ ID NO: 127 - DNA, 126 bp, synthetic construct
  source 1..126: mol_type = other DNA
aagagaggcagaaagaagctgctgtacatcttcaagcagcccttcatgagacccgtgcagaccacccaggaggaggacggctgcagctgcagattccccgaggaggaggagggcggctgcgagctg

SEQ ID NO: 128 - DNA, 336 bp, synthetic construct
  source 1..336: mol_type = other DNA
agagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 129 - DNA, 1128 bp, synthetic construct
  source 1..1128: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgcca

SEQ ID NO: 130 - DNA, 8963 bp, synthetic construct
  source 1..8963: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 131 - DNA, 2944 bp, synthetic construct
  source 1..2944: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtcgagtaggtgtcattctattctggggggtggggtggggcaggacagcaagggggaggattgggaagacaatagcaggcatgctggggatgcggtgggctctatgg

SEQ ID NO: 132 - DNA, 57 bp, synthetic construct
  source 1..57: mol_type = other DNA
cccttaacgtgagttttcgttccactgagcgtcagaccccgtagaaaagatcaaagg

SEQ ID NO: 133 - DNA, 54 bp, synthetic construct
  source 1..54: mol_type = other DNA
gtccaacccggtaagacacgacttatcgccactggcagcagccactggtaacag

SEQ ID NO: 134 - DNA, 53 bp, synthetic construct
  source 1..53: mol_type = other DNA
cacttgccagcgccctagcgcccgctcctttcgctttcttcccttcctttctc

SEQ ID NO: 135 - DNA, 33 bp, synthetic construct
  source 1..33: mol_type = other DNA
gggcgcgtcagcgggtgttggcgggtgtcgggg

SEQ ID NO: 136 - DNA, 8702 bp, synthetic construct
  source 1..8702: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 137 - DNA, 2684 bp, synthetic construct
  source 1..2684: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 138 - DNA, 4395 bp, synthetic construct
  source 1..4395: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 139 - DNA, 4789 bp, synthetic construct
  source 1..4789: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 140 - DNA, 4759 bp, synthetic construct
  source 1..4759: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 141 - DNA, 4762 bp, synthetic construct
  source 1..4762: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 142 - protein, 500 aa, synthetic construct
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 143 - protein, 417 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSG

SEQ ID NO: 144 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 145 - DNA, 1000 bp, synthetic construct
  source 1..1000: mol_type = other DNA
aagccctagtatcaccctgctgggtgggcttcccctaggagaaagctgctactcagattttggtcggctcagttcccccaaccccagccatccaagaatatggtgttactgaacctctcagtgcatgggttggtgggaccctttattgcggaaaagggaaactgaagcccagtgaagagcaatctttgtacaagatcgtagagcctggtagggctggcttgccagggtgggaactggagtgtggattaggtccctgcccttggtgggagctcatactgacaggaggtgagacttgccagtgcctttagaggccactgtccaccccagaccctccactgggagaagcagaggccaggataggctgacttgtatgggtaggggattatttcagtctctaagtcctggccagtgtggacctaggtccctctgagagacactcctatccatggggaggtagcgtgtgacctcaggcagcctaccataatcctggggcttctctggaggaccccacacagatctatcataggataaatctacttgtctgtcgtagatctatctcaactaggaccattcatcagtgggccatttaatcagcccacttcagccaggagggcacaaacatgaagctactttgcactgaatcctatcagtgtggtgagaacattagacatgcctatagggaatgatctgtgtttttggttgatataaaatagagagcaattaattactgtttagtaaatgcagggcagtggaaaagaatctttcaaaagaggatccccacatggaaacaatcaaggtggcttttgtgatgacaagtggggaacgagatactggactaactgagcccatggctgggtagaggggaagctgaggctcatagtgagatgggccagccagaggtcatgaaactagtctggggcataggggggtctctggagatgggggaagcatgggtcctccactcactgctcatccactgccttctcttagacctcgtcct

SEQ ID NO: 146 - DNA, 1490 bp, synthetic construct
  source 1..1490: mol_type = other DNA
gtgaccggcggctctagagcctctgctaaccatgttcatgccttcttctttttcctacaggggggatccgtttatctgcagaattcgcccttgacgtcgccaccatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacgcggactgggcag

SEQ ID NO: 147 - DNA, 1125 bp, synthetic construct
  source 1..1125: mol_type = other DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 148 - DNA, 1000 bp, synthetic construct
  source 1..1000: mol_type = other DNA
gcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggtgagtctgaggggggaggcaggccttttcctgagtagtctggtgggagaatcttagttctgcctttgagtctgatttgggaggcatagcccggcccaggagagtctgatgggagaggcacagccctctctagggaagcgtgaagctggaggcactgccttgacttgcactggattggttaccccagctaatttccactatgtcccttggccccctctgcacttgcctaggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctgactgtacggggcaatctgcccaccctcacccagtcgca

SEQ ID NO: 149 - DNA, 9703 bp, synthetic construct
  source 1..9703: mol_type = other DNA
aaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtaagccctagtatcaccctgctgggtgggcttcccctaggagaaagctgctactcagattttggtcggctcagttcccccaaccccagccatccaagaatatggtgttactgaacctctcagtgcatgggttggtgggaccctttattgcggaaaagggaaactgaagcccagtgaagagcaatctttgtacaagatcgtagagcctggtagggctggcttgccagggtgggaactggagtgtggattaggtccctgcccttggtgggagctcatactgacaggaggtgagacttgccagtgcctttagaggccactgtccaccccagaccctccactgggagaagcagaggccaggataggctgacttgtatgggtaggggattatttcagtctctaagtcctggccagtgtggacctaggtccctctgagagacactcctatccatggggaggtagcgtgtgacctcaggcagcctaccataatcctggggcttctctggaggaccccacacagatctatcataggataaatctacttgtctgtcgtagatctatctcaactaggaccattcatcagtgggccatttaatcagcccacttcagccaggagggcacaaacatgaagctactttgcactgaatcctatcagtgtggtgagaacattagacatgcctatagggaatgatctgtgtttttggttgatataaaatagagagcaattaattactgtttagtaaatgcagggcagtggaaaagaatctttcaaaagaggatccccacatggaaacaatcaaggtggcttttgtgatgacaagtggggaacgagatactggactaactgagcccatggctgggtagaggggaagctgaggctcatagtgagatgggccagccagaggtcatgaaactagtctggggcataggggggtctctggagatgggggaagcatgggtcctccactcactgctcatccactgccttctcttagacctcgtcctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacgcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggtgagtctgaggggggaggcaggccttttcctgagtagtctggtgggagaatcttagttctgcctttgagtctgatttgggaggcatagcccggcccaggagagtctgatgggagaggcacagccctctctagggaagcgtgaagctggaggcactgccttgacttgcactggattggttaccccagctaatttccactatgtcccttggccccctctgcacttgcctaggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctgactgtacggggcaatctgcccaccctcacccagtcgcaggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaa

SEQ ID NO: 150 - protein, 374 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 151 - DNA, 20 bp, synthetic construct
  source 1..20: mol_type = other DNA
tgctggcgatacgcgtccac

SEQ ID NO: 152 - DNA, 1131 bp, synthetic construct
  source 1..1131: mol_type = other DNA
atggcgcttccggtgacagcactgctcctccccttggcgctgttgctccacgcagcaaggccgcaagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 153 - DNA, 348 bp, synthetic construct
  source 1..348: mol_type = other DNA
caagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctcc

SEQ ID NO: 154 - DNA, 120 bp, synthetic construct
  source 1..120: mol_type = other DNA
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 155 - DNA, 336 bp, synthetic construct; identical to SEQ ID NO: 30
  source 1..336: mol_type = other DNA

SEQ ID NO: 156 - DNA, 10884 bp, synthetic construct
  source 1..10884: mol_type = other DNA
atgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattacatgcatagaaggtataatgatc

SEQ ID NO: 157 - protein, 377 aa, synthetic construct
MALPVTALLLPLALLLHAARPQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - DNA, 11208 bp, synthetic construct
  source 1..11208: mol_type = other DNA
tgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggccttaccagtgaccgccttgctcctgccgctggccttgctgctccacgccgccaggccggacgtggtcatgacccagacccctctgagcctgcccgtgagcctgggcgaccaggcttccatctcctgcagaagctcccagagcctggtgcacagcagcggcaacacctacctgcactggtacctgcagaagcccggccagagccccaagctgctgatctacaaggtgtccaacagattttccggcgtgcccgacagattttccggatccggcagcggcaccgacttcacactgaagatcagcagagtggaggccgaggacctgggcgtgtacttctgtagccagagcacacacgtgccttacacctttggcggcggcacaaagctggagatcaagggcggcggcggctccggaggaggaggaagcggaggaggcggctccgaggtgcagctgcagcagtccggccctgacctggtgaagcctggcgcctccatgaagatcagctgcaaggccagcggctactccttcaccgattacaccatgcactgggtgaagcagagccacggcaagaatttcgagtggatcggcctgatcaatccctacaatgacggcaccacctacaatcagaagttcaagggcaaggccacactgacagtggataagagcagctccacagcctacatggagctgctgagcctgacaagcgaggattccgccgtgtactactgcgcctccctggactactggggccagggcacctccgtgaccgtgtcctccaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattaca

SEQ ID NO: 159 - DNA, 1455 bp, synthetic construct
  source 1..1455: mol_type = other DNA
atggccttaccagtgaccgccttgctcctgccgctggccttgctgctccacgccgccaggccggacgtggtcatgacccagacccctctgagcctgcccgtgagcctgggcgaccaggcttccatctcctgcagaagctcccagagcctggtgcacagcagcggcaacacctacctgcactggtacctgcagaagcccggccagagccccaagctgctgatctacaaggtgtccaacagattttccggcgtgcccgacagattttccggatccggcagcggcaccgacttcacactgaagatcagcagagtggaggccgaggacctgggcgtgtacttctgtagccagagcacacacgtgccttacacctttggcggcggcacaaagctggagatcaagggcggcggcggctccggaggaggaggaagcggaggaggcggctccgaggtgcagctgcagcagtccggccctgacctggtgaagcctggcgcctccatgaagatcagctgcaaggccagcggctactccttcaccgattacaccatgcactgggtgaagcagagccacggcaagaatttcgagtggatcggcctgatcaatccctacaatgacggcaccacctacaatcagaagttcaagggcaaggccacactgacagtggataagagcagctccacagcctacatggagctgctgagcctgacaagcgaggattccgccgtgtactactgcgcctccctggactactggggccagggcacctccgtgaccgtgtcctccaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 160 - DNA, 63 bp, synthetic construct
  source 1..63: mol_type = other DNA
atggccttaccagtgaccgccttgctcctgccgctggccttgctgctccacgccgccaggccg

SEQ ID NO: 161 - DNA, 135 bp, synthetic construct
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 162 - DNA, 204 bp, synthetic construct
  source 1..204: mol_type = other DNA
ttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagc

SEQ ID NO: 163 - DNA, 336 bp, synthetic construct
  source 1..336: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 164 - protein, 484 aa, synthetic construct
MALPVTALLLPLALLLHAARPDVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPYTFGGGTKLEIKGGGGSGGGGSGGGGSEVQLQQSGPDLVKPGASMKISCKASGYSFTDYTMHWVKQSHGKNFEWILINPYNDGTTYNQKFKGKATLTVDKSSSTAYMELLSLTSEDSAVYYCASLDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - RNA, 20 nt, synthetic construct
  source 1..20: mol_type = other RNA
ggtcgcggcgccagcacgaa

SEQ ID NO: 166 - RNA, 20 nt, synthetic construct
  source 1..20: mol_type = other RNA
ccgaagcccgggtcatccgg

SEQ ID NO: 167 - RNA, 20 nt, synthetic construct
  source 1..20: mol_type = other RNA
ccgcgacctccggatgaccc

SEQ ID NO: 168 - RNA, 20 nt, synthetic construct
  source 1..20: mol_type = other RNA
cgtgctggcgccgcgacctc

SEQ ID NO: 169 - RNA, 20 nt, synthetic construct
  source 1..20: mol_type = other RNA
cgaaaggaaccacgctggtc

SEQ ID NO: 170 - RNA, 20 nt, synthetic construct
  source 1..20: mol_type = other RNA
cagcgtggttcctttcgtgc

SEQ ID NO: 171 - RNA, 20 nt, synthetic construct
  source 1..20: mol_type = other RNA
gccgcgacctccggatgacc

SEQ ID NO: 172 - RNA, 20 nt, synthetic construct
  source 1..20: mol_type = other RNA
gaaccacgctggtcaggaat

SEQ ID NO: 173 - RNA, 20 nt, synthetic construct
  source 1..20: mol_type = other RNA
cagcacgaaaggaaccacgc

SEQ ID NO: 174 - RNA, 20 nt, synthetic construct
  source 1..20: mol_type = other RNA
gtagcggggccgggaacatg

SEQ ID NO: 175 - RNA, 20 nt, synthetic construct
  source 1..20: mol_type = other RNA
ggtccatctggtcatagaag

SEQ ID NO: 176 - RNA, 20 nt, synthetic construct
  source 1..20: mol_type = other RNA
gctccaggtagccaccttct

SEQ ID NO: 177 - RNA, 20 nt, synthetic construct
  source 1..20: mol_type = other RNA
taggggccccaactccatgg

SEQ ID NO: 178 - RNA, 20 nt, synthetic construct
  source 1..20: mol_type = other RNA
ggcttatgccaatatcggtg

SEQ ID NO: 179 - RNA, 20 nt, synthetic construct
  source 1..20: mol_type = other RNA
aggtgatgaagagaccaggg

SEQ ID NO: 180 - RNA, 20 nt, synthetic construct
  source 1..20: mol_type = other RNA
ggccgagatgtctcgctccg

SEQ ID NO: 181 - RNA, 20 nt, synthetic construct
  source 1..20: mol_type = other RNA
ggattgctcaacaaccatgc

SEQ ID NO: 182 - RNA, 20 nt, synthetic construct
  source 1..20: mol_type = other RNA
gattgctcaacaaccatgct

SEQ ID NO: 183 - RNA, 20 nt, synthetic construct
  source 1..20: mol_type = other RNA
gtgactgacatcaactccaa

SEQ ID NO: 184 - RNA, 20 nt, synthetic construct
  source 1..20: mol_type = other RNA
cacttgggcattaacacttt

SEQ ID NO: 185 - RNA, 20 nt, synthetic construct
  source 1..20: mol_type = other RNA
ttggaaggcctgcatcatga

SEQ ID NO: 186 - RNA, 20 nt, synthetic construct
  source 1..20: mol_type = other RNA
actccaagggattggaattg

SEQ ID NO: 187 - RNA, 20 nt, synthetic construct
  source 1..20: mol_type = other RNA
gctactctctctttctggcc

SEQ ID NO: 188 - RNA, 20 nt, synthetic construct
  source 1..20: mol_type = other RNA
cgcgagcacagctaaggcca

SEQ ID NO: 189 - RNA, 20 nt, synthetic construct
  source 1..20: mol_type = other RNA
ctagggactgcacagtcaat

SEQ ID NO: 190 - RNA, 20 nt, synthetic construct
  source 1..20: mol_type = other RNA
tcgccgctgccgcggggaca

SEQ ID NO: 191 - RNA, 20 nt, synthetic construct
  source 1..20: mol_type = other RNA
gacatggtcctctgcgttca

SEQ ID NO: 192 - RNA, 20 nt, synthetic construct
  source 1..20: mol_type = other RNA
gtccgctccacagccagcaa

SEQ ID NO: 193 - RNA, 20 nt, synthetic construct
  source 1..20: mol_type = other RNA
gttccagggacggggcccac

SEQ ID NO: 194 - RNA, 20 nt, synthetic construct
  source 1..20: mol_type = other RNA
tcgggcctcgctggccgtaa

SEQ ID NO: 195 - RNA, 20 nt, synthetic construct
  source 1..20: mol_type = other RNA
cgtactaagaacgtgccttc

SEQ ID NO: 196 - RNA, 20 nt, synthetic construct
  source 1..20: mol_type = other RNA
gggttccattacggccagcg

SEQ ID NO: 197 - RNA, 20 nt, synthetic construct
  source 1..20: mol_type = other RNA
caggtgttgtcgggcctcgc

SEQ ID NO: 198 - RNA, 20 nt, synthetic construct
  source 1..20: mol_type = other RNA
tactcaatgcgtacattggt

SEQ ID NO: 199 - RNA, 20 nt, synthetic construct
  source 1..20: mol_type = other RNA
aaggctgaccacatccggaa

SEQ ID NO: 200 - RNA, 20 nt, synthetic construct
  source 1..20: mol_type = other RNA
tacattggtggggccacgag

SEQ ID NO: 201 - RNA, 20 nt, synthetic construct
  source 1..20: mol_type = other RNA
ctgtcagtgaaaaccactcg

SEQ ID NO: 202 - RNA, 20 nt, synthetic construct
  source 1..20: mol_type = other RNA
ggtcatcgatgggagcaacg

SEQ ID NO: 203 - RNA, 20 nt, synthetic construct
  source 1..20: mol_type = other RNA
caccaccccgcgggactaga

SEQ ID NO: 204 - RNA, 20 nt, synthetic construct
  source 1..20: mol_type = other RNA
ggtctggcgctcccgctcgg

SEQ ID NO: 205 - RNA, 20 nt, synthetic construct
  source 1..20: mol_type = other RNA
ccaccaccccgcgggactag

SEQ ID NO: 206 - RNA, 20 nt, synthetic construct
  source 1..20: mol_type = other RNA
ttaggggtgccaccaccccg

SEQ ID NO: 207 - RNA, 20 nt, synthetic construct
  source 1..20: mol_type = other RNA
ttcacaccatcacgacgcgt

SEQ ID NO: 208 - RNA, 20 nt, synthetic construct
  source 1..20: mol_type = other RNA
acaccatcacgacgcgtggg

SEQ ID NO: 209 - RNA, 20 nt, synthetic construct
  source 1..20: mol_type = other RNA
ctacgagtctgacgggatcg

SEQ ID NO: 210 - RNA, 20 nt, synthetic construct
  source 1..20: mol_type = other RNA
acgacgcgtgggtggcaagc

SEQ ID NO: 211 - RNA, 20 nt, synthetic construct
  source 1..20: mol_type = other RNA
tgctggcgatacgcgtccac